SEQUENCE LISTING (3 sequences)

SEQ ID NO: 1 - protein, 616 aa, Artificial Sequence
  Description of Artificial Sequence Synthetic polypeptide
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH

SEQ ID NO: 2 - protein, 6 aa, Artificial Sequence
  Description of Artificial Sequence Synthetic peptide
HHHHHH

SEQ ID NO: 3 - protein, 13 aa, Artificial Sequence
  Description of Artificial Sequence Synthetic peptide
  MOD_RES (1)..(1): Beta-alanine
ASARWRFPARPGT

(19) United States
(12) Patent Application Publication
(10) Pub. No.: US 2024/0417405 A1
(43) Pub. Date: Dec. 19, 2024

(54) Substituted Spiro Derivatives

(71) Applicant: Janssen Pharmaceutica NV, Beerse (BE)

(72) Inventors: Olivier Alexis Georges Querolle, Saint Vigor (FR); Xuedong Dai, Shanghai (CN); Wei Cai, Shanghai (CN); Johannes Wilhelmus J. Thuring, Antwerpen (BE); Lichao Fang, Shanghai (CN); Ming Li, Shanghai (CN); Lianzhu Liu, Shanghai (CN); Yingtao Liu, Shanghai (CN); Luoheng Qin, Shanghai (CN); Jianping Wu, Shanghai (CN); Yanping Xu, Noblesville, IN (US); Patrick Rene Angibaud, Longueville (FR); Helene France Solange Colombeo, Guenouville (FR); Isbelle Noelle Constance Pilatte, Louviers (FR); Virginie Sophie Poncelet, Le Manoir sur Seine (FR); Carsten Sven Kramer, Heidelberg (DE); Vineet Pande, Vosselaar (BE); Xiangjun Deng, Shanghai (CN)

(21) Appl. No.: 18/559,689

(22) PCT Filed: May 6, 2022
(86) PCT No.: PCT/CN2022/091066

§ 371 Date: Nov. 8, 2023

(30) Foreign Application Priority Data
May 8, 2021 (WO) PCT/CN2021/092257

(51) Int. Cl. C07D 487/10; A61K 31/53; A61K 31/5377; A61K 31/5386; A61K 31/541; A61K 31/551; A61P 35/02; C07D 519/00

(52) U.S. Cl. CPC C07D 487/10; A61K 31/53; A61K 31/5377; A61K 31/5386; A61K 31/541; A61K 31/551; A61P 35/02; C07D 519/00

(57) ABSTRACT

The present invention relates to pharmaceutical agents useful for therapy and/or prophylaxis in a mammal, pharmaceutical composition comprising such compounds, and their use as menin/MLL protein/protein interaction inhibitors, useful for treating diseases such as cancer, myelodysplastic syndrome (MDS) and diabetes.

### SEQUENCE LISTING

The instant application contains a Sequence Listing which has been submitted electronically in ASCII format and is hereby incorporated by reference in its entirety. Said ASCII copy, created on Dec. 5, 2023, is named JAB7085USPCT2_SL.txt and is 6,278 bytes in size.

### FIELD OF THE INVENTION

The present invention relates to pharmaceutical agents useful for therapy and/or prophylaxis in a mammal, pharmaceutical composition comprising such compounds, and their use as menin/MLL protein/protein interaction inhibitors, useful for treating diseases such as cancer, myelodysplastic syndrome (MDS) and diabetes.

### BACKGROUND OF THE INVENTION

Chromosomal rearrangements affecting the mixed lineage leukemia gene (MLL; MLL1; KMT2A) result in aggressive acute leukemias across all age groups and still represent mostly incurable diseases emphasizing the urgent need for novel therapeutic approaches. Acute leukemias harboring these chromosomal translocations of MLL represent as lymphoid, myeloid or biphenotypic disease and constitute 5 to 10% of acute leukemias in adults and approximately 70% in infants (Marschalek, Br J Haematol 2011. 152(2), 141-54; Tomizawa et al., Pediatr Blood Cancer 2007. 49(2), 127-32).

MLL is a histone methyltransferase that methylates histone H3 on lysine 4 (H3K4) and functions in multiprotein complexes. Use of inducible loss-of-function alleles of Mll1 demonstrated that Mll1 plays an essential role in sustaining hematopoietic stem cells (HSCs) and developing B cells although its histone methyltransferase activity is dispensable for hematopoiesis (Mishra et al., Cell Rep 2014. 7(4), 1239-47).

Fusion of MLL with more than 60 different partners has been reported to date and has been associated with leukemia formation/progression (Meyer et al., Leukemia 2013. 27, 2165-2176). Interestingly, the SET (Su(var)3-9, enhancer of zeste, and trithorax) domain of MLL is not retained in chimeric proteins but is replaced by the fusion partner (Thiel et al., Bioessays 2012. 34, 771-80). Recruitment of chromatin modifying enzymes like Dot1L and/or the pTEFb complex by the fusion partner leads to enhanced transcription and transcriptional elongation of MLL target genes including HOXA genes (e.g. HOXA9) and the HOX cofactor MEIS1 as the most prominent ones. Aberrant expression of these genes in turn blocks hematopoietic differentiation and enhances proliferation.

Menin which is encoded by the Multiple Endocrine Neoplasia type 1 (MEN1) gene is expressed ubiquitously and is predominantly localized in the nucleus. It has been shown to interact with numerous proteins and is, therefore, involved in a variety of cellular processes. The best understood function of menin is its role as an oncogenic cofactor of MLL fusion proteins. Menin interacts with two motifs within the N-terminal fragment of MLL that is retained in all fusion proteins, MBM1 (menin-binding motif 1) and MBM2 (Thiel et al., Bioessays 2012. 34, 771-80). Menin/MLL interaction leads to the formation of a new interaction surface for lens epithelium-derived growth factor (LEDGF). Although MLL directly binds to LEDGF, menin is obligatory for the stable interaction between MLL and LEDGF and the gene specific chromatin recruitment of the MLL complex via the PWWP domain of LEDGF (Cermakova et al., Cancer Res 2014. 15, 5139-51; Yokoyama & Cleary, Cancer Cell 2008. 8, 36-46). Furthermore, numerous genetic studies have shown that menin is strictly required for oncogenic transformation by MLL fusion proteins suggesting the menin/MLL interaction as an attractive therapeutic target. For example, conditional deletion of Men1 prevents leukemogenesis in bone marrow progenitor cells ectopically expressing MLL fusions (Chen et al., Proc Natl Acad Sci 2006. 103, 1018-23). Similarly, genetic disruption of menin/MLL fusion interaction by loss-of-function mutations abrogates the oncogenic properties of the MLL fusion proteins, blocks the development of leukemia in vivo and releases the differentiation block of MLL-transformed leukemic blasts. These studies also showed that menin is required for the maintenance of HOX gene expression by MLL fusion proteins (Yokoyama et al., Cell 2005. 123, 207-18). In addition, small molecule inhibitors of menin/MLL interaction have been developed suggesting druggability of this protein/protein interaction and have also demonstrated efficacy in preclinical models of AML (Borkin et al., Cancer Cell 2015. 27, 589-602; Cierpicki and Grembecka, Future Med Chem 2014. 6, 447-462). Together with the observation that menin is not a requisite cofactor of MLL1 during normal hematopoiesis (Li et al., Blood 2013. 122, 2039-2046), these data validate the disruption of menin/MLL interaction as a promising new therapeutic approach for the treatment of MLL rearranged leukemia and other cancers with an active HOX/MEIS1 gene signature. For example, an internal partial tandem duplication (PTD) within the 5′ region of the MLL gene represents another major aberration that is found predominantly in de novo and secondary AML as well as myeloid dysplasia syndromes. Although the molecular mechanism and the biological function of MLL-PTD is not well understood, new therapeutic targeting strategies affecting the menin/MLL interaction might also prove effective in the treatment of MLL-PTD-related leukemias. Furthermore, castration-resistant prostate cancer has been shown to be dependent on the menin/MLL interaction (Malik et al., Nat Med 2015. 21, 344-52).

MLL protein is also known as Histone-lysine N-methyltransferase 2A (KMT2A) protein in the scientific field (UniProt Accession #Q03164).

Several references describe inhibitors targeting the menin-MLL interaction: WO2011029054, J Med Chem 2016, 59, 892-913 describe the preparation of thienopyrimidine and benzodiazepine derivatives: WO2014164543 describes thienopyrimidine and thienopyridine derivatives; Nature Chemical Biology March 2012, 8, 277-284 and Ren, J.; et al. Bioorg Med Chem Lett (2016), 26(18), 4472-4476 describe thienopyrimidine derivatives; J Med Chem 2014, 57, 1543-1556 describes hydroxy- and aminomethylpiperidine derivatives; Future Med Chem 2014, 6, 447-462 reviews small molecule and peptidomimetic compounds; WO2016195776 describes furo[2,3-d]pyrimidine, 9H-purine, [1,3]oxazolo[5,4-d]pyrimidine, [1,3]oxazolo[4,5-d]pyrimidine, [1,3]thiazolo[5,4-d]pyrimidine, thieno[2,3-b]pyridine and thieno[2,3-d]pyrimidine derivatives; WO2016197027 describes 5,6,7,8-tetrahydropyrido[3,4-d]pyrimidine, 5,6,7,8-tetrahydropyrido]4,3-d]pyrimidine, pyrido[2,3-d]pyrimidine and quinoline derivatives; and WO2016040330 describes thienopyrimidine and thienopyridine compounds. WO2017192543 describes piperidines as Menin inhibitors. WO2017112768, WO2017207387, WO2017214367, WO2018053267 and WO2018024602 describe inhibitors of the menin-MLL interaction. WO2017161002 and WO2017161028 describe inhibitors of menin-MLL. WO2018050686, WO2018050684 and WO2018109088 describe inhibitors of the menin-MLL interaction. WO2018226976 describes methods and compositions for inhibiting the interaction of menin with MLL proteins. WO2019060365 describes substituted inhibitors of menin-MLL. Krivtsov et al., Cancer Cell 2019. No. 6 Vol. 36, 660-673 describes a menin-MLL inhibitor.

WO2020069027 discloses inhibitors of Menin. WO2018175746 discloses methods for treating hematological malignancies and ewing's sarcoma. WO2020045334 discloses azabicyclic derivative used in pharmaceutical compositions. WO2019120209 discloses substituted heterocyclic compounds as menin/MLL protein/protein interaction inhibitors. CN111297863 discloses use of menin-mixed lineage leukemia (MLL) inhibitors. WO2021121327 describes substituted straight chain spiro derivatives and their use as menin/MLL protein/protein interaction inhibitors.

### DESCRIPTION OF THE INVENTION

The present invention concerns novel compounds of Formula (I),

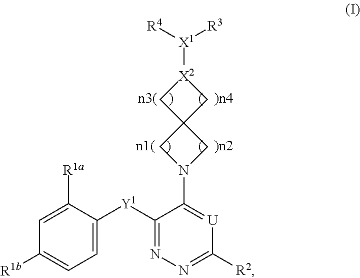

and the tautomers and the stereoisomeric forms thereof, wherein
R^{1a} represents Het;
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl;
R^{1b} represents F or Cl;
Y¹ represents —CR^{5a}R^{5b}—, —O—, —S—, or —NR^{5c}—;
R² is selected from the group consisting of hydrogen, halo, C₁₋₄alkyl, —O—C₁₋₄alkyl, and —NR^{7a}R^{7b}; U represents N or CH;
n1, n2, n3 and n4 are each independently selected from 1 and 2;
X¹ represents CH, and X² represents N;
R⁴ represents C₁₋₅alkyl;

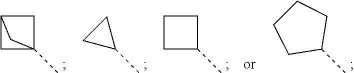

R^{5a}, R^{5b}, R^{5c}, R^{7a}, and R^{7b}, are each independently selected from the group consisting of hydrogen, C₁₋₄alkyl and C₃₋₆cycloalkyl;
R³ is selected from the group consisting of Het¹, Het², Cy² and —C₁₋₆alkyl-NR^{xc}R^{xd};
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, and cyano;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of Het³; Het⁴; —C(═O)—NH—Cy¹; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH,
—O—C₁₋₄alkyl, —C(═O)—NH—C₁₋₄alkyl, —C(═O)—NH—C₁₋₄alkyl-C₃₋₆cycloalkyl,
—C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl,
—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and
—NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, halo, cyano, —NR^{11a}R^{11b} Het^{3a}, and Het^{6a};
Het³, Het^{3a}, Het⁵ and Het^{5a} each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂; or a bicyclic C-linked 6- to 11-membered fully saturated heterocyclyl containing one, two or three heteroatoms each independently selected from O, S, and N,
wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl,
—O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{6b} and Het^{8b} each independently represent a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl; wherein said C₃₋₇cycloalkyl is optionally substituted with one, two, three or four substituents each independently selected from the group consisting of halo, R⁶, Het^{6a}, Het^{6b},
—NR^{9a}R^{9b}, —OH, C₁₋₄alkyl, and C₁₋₄alkyl substituted with one or two substituents each independently selected from the group consisting of Het^{3a}, Het^{6a}, Het^{6b}, and
—NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or
C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer, myelodysplastic syndrome (MDS) and diabetes.

In a particular embodiment, the invention relates to a compound of Formula (I), a pharmaceutically acceptable salt, or a solvate thereof, for use in the treatment or in the prevention of cancer.

In a specific embodiment said cancer is selected from leukemias, myeloma or a solid tumor cancer (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (LHCL), MLL-rearranged leukemias, MLL-PTD leukemias, MLL amplified leukemias, MLL-positive leukemias, leukemias exhibiting HOX/MEIS1 gene expression signatures etc.

The invention also relates to the use of a compound of Formula (I), a pharmaceutically acceptable salt, or a solvate thereof, in combination with an additional pharmaceutical agent for use in the treatment or prevention of cancer, myelodysplastic syndrome (MDS) and diabetes.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer, myelodysplastic syndrome (MDS) and diabetes.

Additionally, the invention relates to a method of treating or preventing a cell proliferative disease in a warm-blooded animal which comprises administering to the said animal an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x_y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain saturated hydrocarbon radical having from 1 to 4 carbon atoms, such as methyl, ethyl, n-propyl, isopropyl, n-butyl, s-butyl, t-butyl and the like.

The term ‘C₃₋₆cycloalkyl’ as used herein as a group or part of a group defines a saturated, cyclic hydrocarbon radical having from 3 to 6 carbon atoms, such as cyclopropyl, cyclobutyl, cyclopentyl and cyclohexyl.

The term ‘C₃₋₇cycloalkyl’ as used herein as a group or part of a group defines a saturated, cyclic hydrocarbon radical having from 3 to 7 carbon atoms, such as cyclopropyl, cyclobutyl, cyclopentyl, cyclohexyl and cycloheptyl.

It will be clear for the skilled person that S(═O)₂ or SO₂ represents a sulfonyl moiety.

It will be clear for the skilled person that CO or C(═O) represents a carbonyl moiety.

It will be clear for the skilled person that a group such as —CRR— represents

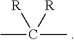

An example of such a group is —CR^{5a}R^{5b}—.

It will be clear for the skilled person that a group such as —NR— represents

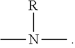

An example of such a group is —NR^{5c}—.

The term ‘monocyclic C-linked 4- to 7-membered fully saturated heterocyclyl containing one N-atom and optionally one or two additional heteroatoms each independently selected from O, S, and N’, defines a fully saturated, cyclic hydrocarbon radical having from 4 to 7 ring members and containing at least 1 nitrogen atom and optionally one or two additional heteroatoms each independently selected from O, S, and N, such as for example C-linked azetidinyl, C-linked pyrrolidinyl, C-linked morpholinyl and C-linked piperidinyl. The term ‘monocyclic N-linked 4- to 7-membered fully saturated heterocyclyl containing one N-atom and optionally one or two additional heteroatoms each independently selected from O, S, and N’, is defined similar but is attached to the remainder of the molecule of formula (I) via a nitrogen atom. Examples are N-linked azetidinyl, N-linked pyrrolidinyl, N-linked morpholinyl, N-linked thiomorpholinyl, N-linked piperazinyl, N-linked 1,4-diazepanyl, and N-linked piperidinyl. Two R groups taken together to form together with the N-atom to which they are attached a 4- to 7-membered monocyclic fully saturated heterocyclyl containing one N-atom and optionally one additional heteroatom selected from O, S, and N, are defined similar.

The term ‘monocyclic C-linked 4- to 7-membered fully saturated heterocyclyl containing one, two or three heteroatoms each independently selected from O, S, and N’, defines a fully saturated, cyclic hydrocarbon radical having from 4 to 7 ring members and containing one, two or three heteroatoms each independently selected from O, S, and N, such as for example C-linked azetidinyl, C-linked pyrrolidinyl, C-linked morpholinyl, C-linked tetrahydrofuranyl, C-linked thiolanyl, C-linked oxetanyl, C-linked thietanyl, C-linked tetrahydropyranyl, C-linked tetrahydrothiopyranyl, and C-linked piperidinyl. The term ‘monocyclic N-linked 4- to 7-membered fully saturated heterocyclyl containing two N-atoms and optionally one additional heteroatom selected from O, S, and N’, defines a fully saturated, cyclic hydrocarbon radical having from 4 to 7 ring members and containing 2 nitrogen atoms and optionally one additional heteroatom selected from O, S, and N, such as for example N-linked piperazinyl, and N-linked 1,4-diazepanyl.

For clarity, the 4- to 7-membered fully or partially saturated heterocyclyls have from 4 to 7 ring members including the heteroatoms.

Non-limiting examples of ‘monocyclic 5- or 6-membered aromatic rings containing one, two or three nitrogen atoms and optionally a carbonyl moiety’, include, but are not limited to pyrazolyl, imidazolyl, pyridinyl, pyridazinyl, pyrimidinyl, pyrazinyl, 1H-1,2,4-triazolyl, 4H-1,2,4-triazolyl, 1,2,4-triazinyl, 1,2-dihydro-2-oxo-5-pyrimidinyl, 1,2-dihydro-2-oxo-6-pyridinyl, 1,2-dihydro-2-oxo-4-pyridinyl, and 1,6-dihydro-6-oxo-3-pyridazinyl.

The skilled person will understand that a 5- or 6-membered monocyclic aromatic ring containing one two or three nitrogen atoms and a carbonyl moiety includes, but is not limited to

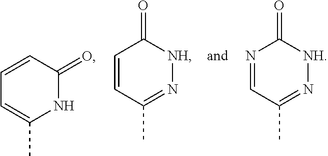

Non-limiting examples of ‘monocyclic C-linked 5- or 6-membered aromatic rings containing one, two or three heteroatoms each independently selected from O, S, and N’, include, but are not limited to C-linked pyrazolyl, C-linked imidazolyl, C-linked pyridinyl, C-linked triazolyl, C-linked pyridazinyl, C-linked pyrimidinyl, C-linked oxazolyl, C-linked furanyl, C-linked isothiazolyl, C-linked thiazolyl, C-linked thiadiazolyl, C-linked oxadiazolyl, or C-linked pyrazinyl.

Within the context of this invention, bicyclic C-linked 6- to 11-membered fully saturated heterocyclyl groups, include fused, spiro and bridged bicycles.

Within the context of this invention, bicyclic N-linked 6- to 11-membered fully saturated heterocyclyl groups, include fused, spiro and bridged bicycles.

Fused bicyclic groups are two cycles that share two atoms and the bond between these atoms.

Spiro bicyclic groups are two cycles that are joined at a single atom.

Bridged bicyclic groups are two cycles that share more than two atoms.

Examples of bicyclic C-linked 6- to 11-membered fully saturated heterocyclyl containing one N-atom and optionally one or two additional heteroatoms each independently selected from O, S, and N, include, but are not limited to

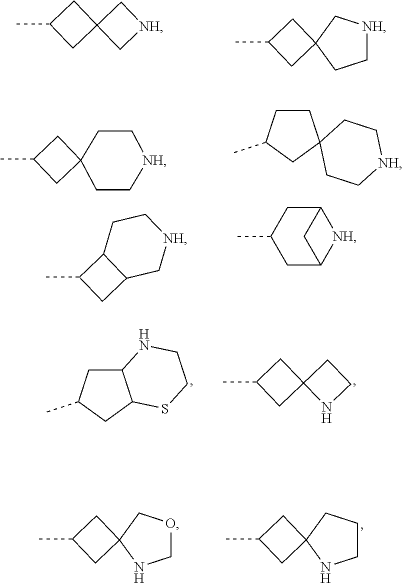

and the like.

Examples of bicyclic C-linked 6- to 11-membered fully saturated heterocyclyl containing one, two or three heteroatoms each independently selected from O, S, and N, include, but are not limited to

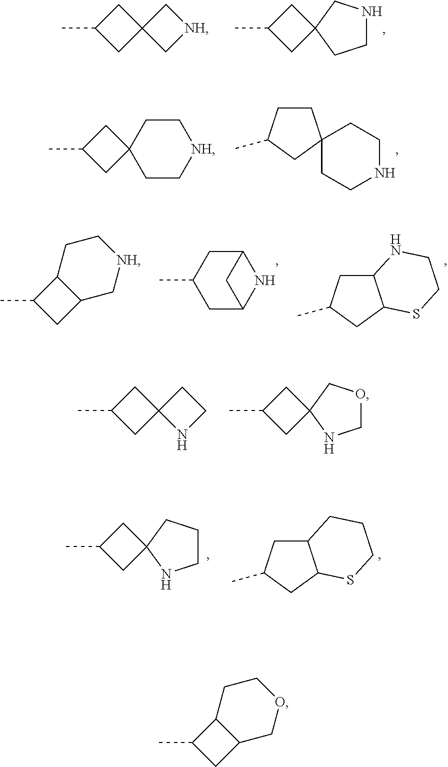

and the like.

Examples of bicyclic N-linked 6- to 11-membered fully saturated heterocyclyl containing one N-atom and optionally one or two additional heteroatoms each independently selected from O, S, and N, include, but are not limited to

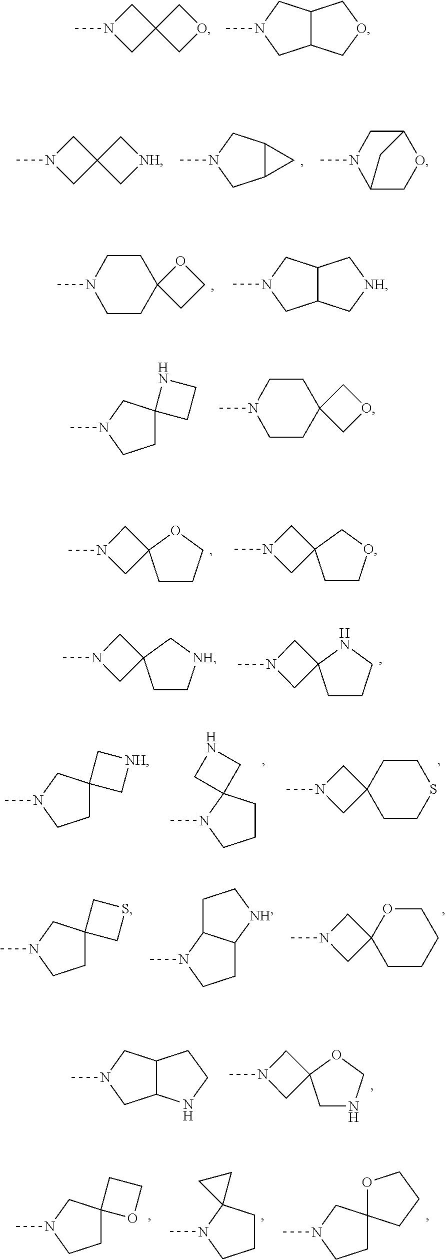

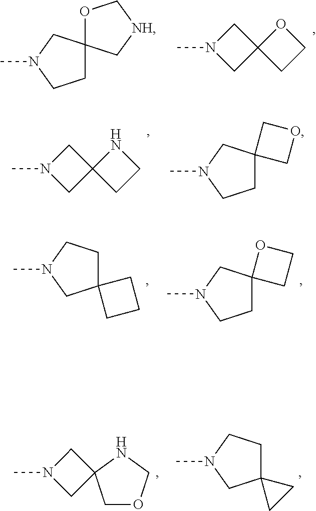

and the like.

Examples of fused bicyclic C-linked 9- or 10-membered aromatic ring containing one, two, three or four heteroatoms each independently selected from O, S, and N, include but are not limited to

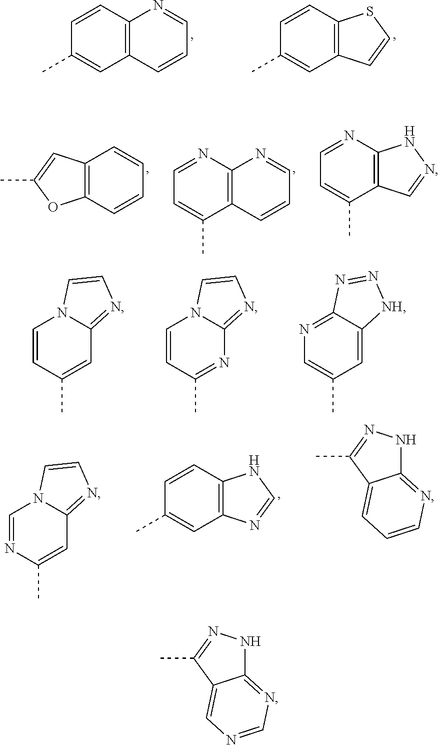

and the like.

Whenever substituents are represented by chemical structure, such as for example

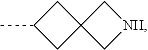

‘—’ represents the bond of attachment to the remainder of the molecule of Formula (I).

When any variable occurs more than one time in any constituent, each definition is independent.

When any variable occurs more than one time in any formula (e.g. Formula (I)), each definition is independent.

In this context, it will also be clear that a term like “optionally substituted with one, two or three substituents selected from the group consisting of” is equivalent to “optionally substituted with one, two or three substituents each independently selected from the group consisting of”.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture. In a particular embodiment, when the number of substituents is not explicitly specified, the number of substituents is one.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Within the context of this invention ‘saturated’ means ‘fully saturated’, if not otherwise specified.

Unless otherwise specified or clear from the context, aromatic rings and heterocyclyl groups, can be attached to the remainder of the molecule of Formula (I) through any available ring carbon atom (C-linked) or nitrogen atom (N-linked).

Unless otherwise specified or clear from the context, aromatic rings and heterocyclyl groups, may optionally be substituted, where possible, on carbon and/or nitrogen atoms according to the embodiments.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2 r and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Some of the compounds according to Formula (I) may also exist in their tautomeric form. Such forms in so far as they may exist, although not explicitly indicated in the above Formula (I) are intended to be included within the scope of the present invention. It follows that a single compound may exist in both stereoisomeric and tautomeric form.

For example

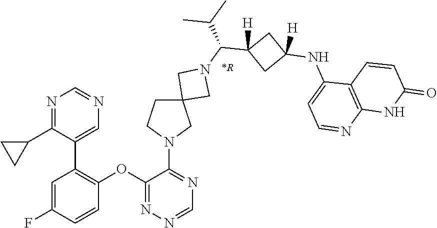 [SMILES: CC(C)[C@H]([C@H]1C[C@@H](Nc2ccnc3[nH]c(=O)ccc23)C1)N1CC2(CCN(c3ncnnc3Oc3ccc(F)cc3-c3cncnc3C3CC3)C2)C1]

also covers the other tautomeric form

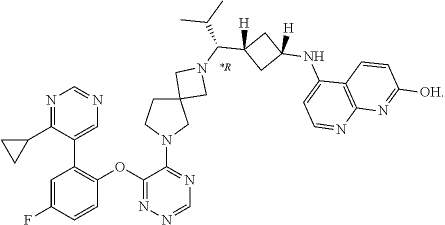 [SMILES: CC(C)[C@H]([C@H]1C[C@@H](Nc2ccnc3nc(O)ccc23)C1)N1CC2(CCN(c3ncnnc3Oc3ccc(F)cc3-c3cncnc3C3CC3)C2)C1]

For example

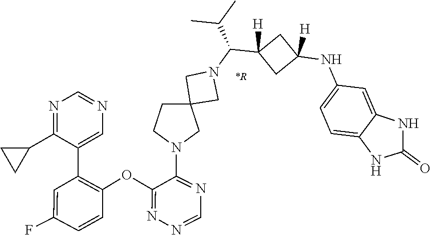 [SMILES: CC(C)[C@H]([C@H]1C[C@@H](Nc2ccc3[nH]c(=O)[nH]c3c2)C1)N1CC2(CCN(c3ncnnc3Oc3ccc(F)cc3-c3cncnc3C3CC3)C2)C1]

also covers the other tautomeric form

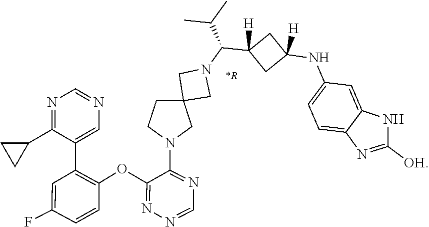 [SMILES: CC(C)[C@H]([C@H]1C[C@@H](Nc2ccc3nc(O)[nH]c3c2)C1)N1CC2(CCN(c3ncnnc3Oc3ccc(F)cc3-c3cncnc3C3CC3)C2)C1]

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I) and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% c by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form.

Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸F, ³²P, ³³P, ³⁵S, ¹⁸F, ³⁶Cl, ¹²²I, ¹²³I, ¹²⁵I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein
R^{1a} represents Het;
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl;
R^{1b} represents F or Cl;
Y¹ represents —CR^{5a}R^{5b}—, —O—, —S—, or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents C₁₋₅alkyl;

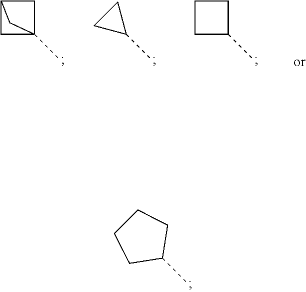

R^{5a}, R^{5b}, R^{5c}, R^{7a}, and R^{7b}, are each independently selected from the group consisting of hydrogen, C₁₋₄alkyl and C₃₋₆cycloalkyl;
R³ is selected from the group consisting of Het¹, Het², Cy² and —C₁₋₆alkyl-NR^{xc}R^{xd};
R^{xc} represents Cy¹; Het⁵; —C₁₋₆alkyl-Cy¹; —C₁₋₆alkyl-Het³; —C₁₋₆alkyl-Het⁴; or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, and cyano;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which is substituted on one nitrogen atom with R^{6a};
R⁶ is selected from the group consisting of
Het³; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl,
—C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and
—NH—S(═O)₂—C₁₋₄alkyl;
R^{6a} represents C₁₋₆alkyl substituted with one substituent selected from the group consisting of —NR^{11a}R^{11b}, Het^{3a}, and Het^{6a};
R⁸ represents C₁₋₆alkyl optionally substituted with one, two or three substituents each independently selected from —OH, halo, cyano, —NR^{11a}R^{11b}, Het^{3a}, and Het^{6a};
Het³ and Het⁵ each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂; or a bicyclic C-linked 6- to 11-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het^{3a} and Het^{5a} each independently represent a monocyclic C-linked 4- to 7-membered fully saturated heterocyclyl containing one N-atom, and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom, and optionally one or two additional heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N; wherein said 5-membered aromatic ring is optionally substituted on one nitrogen atom with C₁₋₄alkyl; and wherein said 5- or 6-membered aromatic ring is optionally substituted on one carbon atom with —OH;
Het^{6a} and Het⁸ each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and
—(C═O)—NR^{10a}R^{10b};
Het^{8a} each independently represent a monocyclic N-linked 4- to 7-membered fully saturated heterocyclyl containing two N-atoms and optionally one additional heteroatom selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —(C═O)—NR^{10a}R^{10b}, —O—C₃₋₆cycloalkyl, —S(═O)₂—C₁₋₄alkyl, cyano, C₁₋₄alkyl,
C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{6b} represents a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl substituted with one or two substituents each independently selected from the group consisting of —NR^{9a}R^{9b}; Het^{6a}; Het^{6b}; and C₁₋₆alkyl substituted with one or two substituents each independently selected from the group consisting of Het^{3a}, Het^{6a}, Het^{6b}, and —NR^{9a}R^{9b}; and said C₃₋₇cycloalkyl is optionally substituted with one or two additional substituents each independently selected from the group consisting of halo, R⁶, C₁₋₄alkyl, and —OH;
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; Het⁵; —C₁₋₄alkyl-R¹⁶; —C(═O)—C₁₋₄alkyl-Het^{3a}; —C(═O)—R¹⁴; C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het^{8a}; or
C₁₋₄alkyl substituted with one, two or three substituents selected from the group consisting of —NR^{13a}R^{13b} and Het^{8a};
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

The present invention relates in particular to compounds of Formula (I) as defined herein, and the tautomers and the stereoisomeric forms thereof, wherein
R^{1a} represents Het;
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of cyano, and C₁₋₄alkyl;
R^{1b} represents F;
Y¹ represents —O—;
R² represents hydrogen;
U represents N;
n1, n2, n3 and n4 are each independently selected from 1 and 2;
X¹ represents CH, and X² represents N;
R⁴ represents C₁₋₅alkyl; or

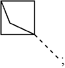

R³ is selected from the group consisting of Het¹ and Cy²;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one, two, three or four substituents each independently selected from the group consisting of oxo and
—OH;
R⁶ and R^{6a} are each independently selected from the group consisting of
Het⁴; C₁₋₆alkyl optionally substituted with one or two substituents each independently selected from the group consisting of Het³, Het^{6a}, and Cy¹; and
C₃₋₆cycloalkyl;
R⁸ represents —O—C₁₋₆alkyl;
Het³, Het^{3a}, Het⁵ and Het^{5a} each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl,
—O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, and —O—C₁₋₄alkyl; and wherein said heterocyclyl is optionally substituted on one nitrogen with a substituent selected from the group consisting of —C(═O)—C₁₋₄alkyl and
—(C═O)—NR^{10a}R^{10b};
Het^{6b} and Het^{8b} each independently represent a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, and C₁₋₄alkyl;
Het⁹ represents a monocyclic C-linked 5- or 6-membered aromatic ring containing one, two or three heteroatoms each independently selected from O, S, and N; wherein said aromatic ring is optionally substituted on one or two carbon atoms with C₁₋₄alkyl;
Cy¹ represents C₃₋₆cycloalkyl optionally substituted with one, two or three substituents selected from the group consisting of —OH and C₁₋₄alkyl;
Cy² represents C₃₋₇cycloalkyl; wherein said C₃₋₇cycloalkyl is optionally substituted with one, two, three or four substituents each independently selected from the group consisting of R⁶, Het^{6a}, Het^{6b},
—NR^{9a}R^{9b}, —OH, and C₁₋₄alkyl;
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; Het⁵; Het⁷; —C₁₋₄alkyl-R¹⁶; —C(═O)—C₁₋₄alkyl-Het^{3a}; —C(═O)—R¹⁴; and
C₁₋₄alkyl substituted with one, two or three substituents selected from the group consisting of halo, —OH, and —O—C₁₋₄alkyl;
R^{11a}, R^{11b}, R^{13a}, R^{13b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents —O—C₁₋₄alkyl; C₃₋₆cycloalkyl substituted with one, two or three substituents selected from the group consisting of —O—C₁₋₄alkyl and halo; or
C₁₋₄alkyl substituted with one, two or three substituents selected from the group consisting of —O—C₁₋₄alkyl, —NR^{13a}R^{13b}, and cyano;
R¹⁶ represents —C(═O)—NR^{17a}R^{17b} or —S(═O)₂—C₁₋₄alkyl;
and the pharmaceutically acceptable salts and the solvates thereof.

The present invention relates in particular to compounds of Formula (I) as defined herein, and the tautomers and the stereoisomeric forms thereof, wherein
R^{1a} represents Het;
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of cyano and C₁₋₄alkyl;
R^{1b} represents F; Y¹ represents —O—; R² represents hydrogen; U represents N;
n1, n2, n3 and n4 are each independently selected from 1 and 2;
X¹ represents CH, and X² represents N;
R⁴ represents C₁₋₅alkyl or

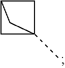

or;
R³ is selected from the group consisting of Het¹ and Cy²;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with R⁶; and wherein said heterocyclyl is optionally substituted on one or two carbon atoms with in total one, two, three or four substituents each independently selected from the group consisting of oxo and —OH;
R⁶ is selected from the group consisting of
C₁₋₆alkyl optionally substituted with one or two substituents each independently selected from the group consisting of Het³, Het^{6a}, and Cy¹; and
C₃₋₆cycloalkyl;
R^{6a} represents C₁₋₆alkyl substituted with one substituent selected from the group consisting of Het^{3a} and Het⁶a;
Het³ and Het⁵ each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl;
Het³a represents a monocyclic C-linked 4- to 7-membered fully saturated heterocyclyl containing one N-atom, and optionally one or two additional heteroatoms each independently selected from O, S, and N, wherein said S-atom might be substituted to form S(═O) or S(═O)₂;
wherein said heterocyclyl is optionally substituted on one carbon atom with C₁₋₄alkyl;
Het⁴ and Het⁷ each independently represent a monocyclic C-linked 5- or 6-membered aromatic ring containing one, two or three heteroatoms each independently selected from O, S, and N; wherein said 5-membered aromatic ring is optionally substituted on one nitrogen atom with C₁₋₄alkyl; and wherein said 5- or 6-membered aromatic ring is optionally substituted on one carbon atom with —OH;
Het^{6a} and Het⁸ each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—(C═O)—NR^{10a}R^{10b}, —O—C₃₋₆cycloalkyl, —S(═O)₂—C₁₋₄alkyl, cyano, C₁₋₄alkyl,
—C₁₋₄alkyl-OH, and —O—C₁₋₄alkyl; and wherein said heterocyclyl is optionally substituted on one nitrogen with a substituent selected from the group consisting of
—C(═O)—C₁₋₄alkyl and —(C═O)—NR^{10a}R^{10b};
Het^{6b} represents a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, and C₁₋₄alkyl;
Cy¹ represents C₃₋₆cycloalkyl optionally substituted with one, two or three substituents selected from the group consisting of —OH and C₁₋₄alkyl;
Cy² represents C₃₋₇cycloalkyl substituted with one or two substituents each independently selected from the group consisting of —NR^{9a}R^{9b}; Het^{6a}; and Het^{6b}; and said C₃₋₇cycloalkyl is optionally substituted with one or two additional substituents each independently selected from the group consisting of R⁶, C₁₋₄alkyl, and —OH;
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; Het⁵; —C₁₋₄alkyl-R¹⁶; —C(═O)—C₁₋₄alkyl-Het^{3a}; —C(═O)—R¹⁴; C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of —OH and —O—C₁₋₄alkyl;
R^{11a}, R^{11b}, R^{13a}, R^{13b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents C₁₋₄alkyl substituted with one, two or three —NR^{13a}R^{13b} substituents;
R¹⁶ represents —C(═O)—NR^{17a}R^{17b} or —S(═O)₂—C₁₋₄alkyl;
and the pharmaceutically acceptable salts and the solvates thereof.

The present invention relates in particular to compounds of Formula (I) as defined herein, and the tautomers and the stereoisomeric forms thereof, wherein
R^{1a} represents Het;
Het represents a monocyclic 5- or 6-membered aromatic ring containing one, two or three nitrogen atoms; wherein said monocyclic 5- or 6-membered aromatic ring is substituted with one C₃₋₆cycloalkyl and wherein said monocyclic 5- or 6-membered aromatic ring is optionally substituted with one C₁₋₄alkyl;
R^{1b} represents F; Y¹ represents —O—; R² represents hydrogen; U represents N;
n1 is 1, n2 is 2, n3 is 1, and n4 is 1;
X¹ represents CH, and X² represents N;
R⁴ represents isopropyl;
R³ represents Cy²;
Het^{6a} represents a monocyclic N-linked 4- to 7-membered fully saturated heterocyclyl containing one N-atom;
Het^{6b} represents a fused bicyclic N-linked 6- to 11-membered fully saturated heterocyclyl containing one N-atom and optionally one additional heteroatom selected from O and N;
wherein said heterocyclyl is optionally substituted on one nitrogen with —C(═O)—C₁₋₄alkyl;
Cy² represents C₃₋₇cycloalkyl substituted with one substituent selected from the group consisting of Het^{6a}, Het^{6b}, and —NR^{9a}R^{9b};
R^{9a} and R^{9b} are each independently selected from C₁₋₄alkyl;
and the pharmaceutically acceptable salts and the solvates thereof.

The present invention relates in particular to compounds of Formula (I) as defined herein, and the tautomers and the stereoisomeric forms thereof, wherein
R^{1a} represents Het;
Het represents

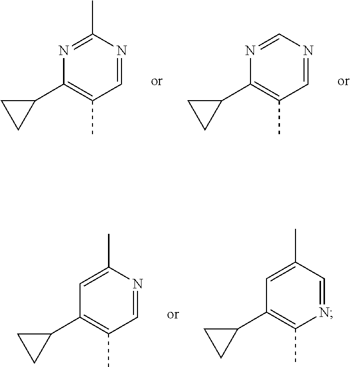

R^{1b} represents F; Y¹ represents —O—; R² represents hydrogen; U represents N;
n1 is 1, n2 is 2, n3 is 1, and n4 is 1;
X¹ represents CH, and X² represents N;
R⁴ represents isopropyl;
R³ represents Cy²;
Het^{6a} represents a monocyclic N-linked 4- to 7-membered fully saturated heterocyclyl containing one N-atom;
Het^{6b} represents a fused bicyclic N-linked 6- to 11-membered fully saturated heterocyclyl containing one N-atom and optionally one additional heteroatom selected from O and N;
wherein said heterocyclyl is optionally substituted on one nitrogen with —C(═O)—C₁₋₄alkyl;
Cy² represents cyclobutyl substituted with one substituent selected from the group consisting of Het^{6a}, Het^{6b}, and —NR^{9a}R^{9b};
R^{9a} and R^{9b} are each independently selected from C₁₋₄alkyl;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein
R^{1b} represents F.

In an embodiment, the present invention relates to those compounds of Formula (I) and the pharmaceutically acceptable salts, and the solvates thereof, or any subgroup thereof as mentioned in any of the other embodiments, wherein
R² represents hydrogen.

In an embodiment, the present invention relates to those compounds of Formula (I) and the pharmaceutically acceptable salts, and the solvates thereof, or any subgroup thereof as mentioned in any of the other embodiments, wherein n1 is 1, n2 is 2, n3 is 1, and n4 is 1.

In an embodiment, the present invention relates to those compounds of Formula (I) and the pharmaceutically acceptable salts, and the solvates thereof, or any subgroup thereof as mentioned in any of the other embodiments, wherein Y¹ represents —O—.

In an embodiment, the present invention relates to those compounds of Formula (I) and the pharmaceutically acceptable salts, and the solvates thereof, or any subgroup thereof as mentioned in any of the other embodiments, wherein
Y¹ represents —O—; and U represents N.

In an embodiment, the present invention relates to those compounds of Formula (I) and the pharmaceutically acceptable salts, and the solvates thereof, or any subgroup thereof as mentioned in any of the other embodiments, wherein U represents N.

In an embodiment, the present invention relates to those compounds of Formula (I) and the pharmaceutically acceptable salts, and the solvates thereof, or any subgroup thereof as mentioned in any of the other embodiments, wherein
Y¹ represents —O—; U represents N;
R^{1b} represents F; and R² represents hydrogen.

In an embodiment, the present invention relates to those compounds of Formula (I) and the pharmaceutically acceptable salts, and the solvates thereof, or any subgroup thereof as mentioned in any of the other embodiments, wherein
Y¹ represents —O—; U represents N;
R^{1b} represents F; R² represents hydrogen; and R⁴ represents isopropyl.

In an embodiment, the present invention relates to those compounds of Formula (I) and the pharmaceutically acceptable salts, and the solvates thereof, or any subgroup thereof as mentioned in any of the other embodiments, wherein
R⁴ represents isopropyl.

In an embodiment, the present invention relates to those compounds of Formula (I) and the pharmaceutically acceptable salts, and the solvates thereof, or any subgroup thereof as mentioned in any of the other embodiments, wherein
R⁴ represents

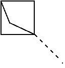

In an embodiment, the present invention relates to those compounds of Formula (I) and the pharmaceutically acceptable salts, and the solvates thereof, or any subgroup thereof as mentioned in any of the other embodiments, wherein
R⁴ represents isopropyl; or

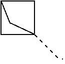

In an embodiment, the present invention relates to those compounds of Formula (I) and the pharmaceutically acceptable salts, and the solvates thereof, or any subgroup thereof as mentioned in any of the other embodiments, wherein
R⁶ and R^{6a} are each independently selected from the group consisting of
Het⁴; C₁₋₆alkyl optionally substituted with one or two substituents each independently selected from the group consisting of Het³, and Cy¹; and C₃₋₆cycloalkyl;
Het³, Het^{3a}, Het⁵ and Het^{5a} each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂.

In an embodiment, the present invention relates to those compounds of Formula (I) and the pharmaceutically acceptable salts, and the solvates thereof, or any subgroup thereof as mentioned in any of the other embodiments, wherein Het represents

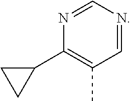

In an embodiment, the present invention relates to those compounds of Formula (I) and the pharmaceutically acceptable salts, and the solvates thereof, or any subgroup thereof as mentioned in any of the other embodiments, wherein Het represents

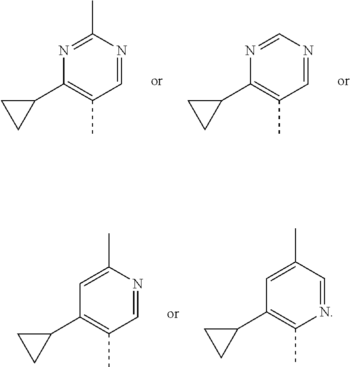

In an embodiment, the present invention relates to those compounds of Formula (I) and the pharmaceutically acceptable salts, and the solvates thereof, or any subgroup thereof as mentioned in any of the other embodiments, wherein Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}C₁₋₄alkyl, oxo, —NR^{9a}R^{9b} and —OH.

In an embodiment, the present invention relates to those compounds of Formula (I) and the pharmaceutically acceptable salts, and the solvates thereof, or any subgroup thereof as mentioned in any of the other embodiments, wherein Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of oxo and —OH.

In an embodiment, the present invention relates to those compounds of Formula (I) and the pharmaceutically acceptable salts, and the solvates thereof, or any subgroup thereof as mentioned in any of the other embodiments, wherein Het¹ represents a bicyclic C-linked 6- to 11-membered fully saturated heterocyclyl containing at least 1 N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}C₁₋₄alkyl, oxo, —NR^{9a}R^{9b} and —OH.

In an embodiment, the present invention relates to those compounds of Formula (I) and the pharmaceutically acceptable salts, and the solvates thereof, or any subgroup thereof as mentioned in any of the other embodiments, wherein Het¹ represents a bicyclic C-linked 6- to 11-membered fully saturated heterocyclyl containing at least 1 N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of oxo and —OH.

In an embodiment, the present invention relates to those compounds of Formula (I) and the pharmaceutically acceptable salts, and the solvates thereof, or any subgroup thereof as mentioned in any of the other embodiments, wherein R⁶ is selected from the group consisting of Het⁴; C₃₋₆cycloalkyl; and C₁₋₆alkyl optionally further substituted with one or two substituents each independently selected from the group consisting of Het³ and Cy¹.

In an embodiment, the present invention relates to those compounds of Formula (I) and the pharmaceutically acceptable salts, and the solvates thereof, or any subgroup thereof as mentioned in any of the other embodiments, wherein
R⁶ and R^{6a} are each independently selected from the group consisting of
Het³; Het⁴; —C(═O)—NH—Cy¹; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl,
—C(═O)—OH, —NR^{11a}R^{11b} and —NH—S(═O)₂—C₁₋₄alkyl; and
C₃₋₆cycloalkyl substituted by one or two substituents each independently selected from the group consisting of —CN, —OH, —O—C₁₋₄alkyl, —C(═O)—NH—C₁₋₄alkyl,
—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and
—NH—S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein
R⁶ is selected from the group consisting of
Het³; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl,
—C(═O)—OH, —NR^{11a}R^{11b}, and —NH—S(═O)₂—C₁₋₄alkyl; and
C₃₋₆cycloalkyl substituted by one or two substituents each independently selected from the group consisting of —CN, —OH, —O—C₁₋₄alkyl, —C(═O)—NH—C₁₋₄alkyl,
—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and
—NH—S(═O)₂—C₁₋₄alkyl;
R^{6a} represents C₁₋₆alkyl substituted with one substituent selected from the group consisting of —NR^{11a}R^{11b}, Het^{3a}, and Het^{6a}.

In an embodiment, the present invention relates to those compounds of Formula (I) and the pharmaceutically acceptable salts, and the solvates thereof, or any subgroup thereof as mentioned in any of the other embodiments, wherein
R⁶ and R^{6a} are each independently selected from the group consisting of
Het⁴; C₁₋₆alkyl optionally substituted with one or two substituents each independently selected from the group consisting of Het³, Het^{6a}, and Cy¹.

In an embodiment, the present invention relates to those compounds of Formula (I) and the pharmaceutically acceptable salts, and the solvates thereof, or any subgroup thereof as mentioned in any of the other embodiments, wherein
R⁶ is selected from the group consisting of Het⁴; C₁₋₆alkyl optionally substituted with one or two substituents each independently selected from the group consisting of Het³, Het^{6a}, and Cy¹;
R^{6a} represents C₁₋₆alkyl substituted with one substituent selected from the group consisting of Het^{3a} and Het^{6a}.

In an embodiment, the present invention relates to those compounds of Formula (I) and the pharmaceutically acceptable salts, and the solvates thereof, or any subgroup thereof as mentioned in any of the other embodiments, wherein R⁶ is selected from the group consisting of Het⁴; and C₁₋₆alkyl optionally further substituted with one or two substituents each independently selected from the group consisting of Het³ and Cy¹.

In an embodiment, the present invention relates to those compounds of Formula (I) and the pharmaceutically acceptable salts, and the solvates thereof, or any subgroup thereof as mentioned in any of the other embodiments, wherein Het¹ represents

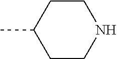

optionally substituted on the nitrogen as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het³ represents

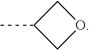

In an embodiment, the present invention relates to those compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one, two or three nitrogen atoms; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one, two or three nitrogen atoms; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl; and
R^{1b} represents F.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one or two nitrogen atoms; wherein said monocyclic 5- or 6-membered aromatic ring is substituted with one C₃₋₆cycloalkyl and wherein said monocyclic 5- or 6-membered aromatic ring is optionally substituted with one cyano.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 5- or 6-membered aromatic ring containing one or two nitrogen atoms; wherein said monocyclic 5- or 6-membered aromatic ring is substituted with one C₃₋₆cycloalkyl and wherein said monocyclic 5- or 6-membered aromatic ring is optionally substituted with one cyano; and
R^{1b} represents F.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 6-membered aromatic ring containing one, two or three nitrogen atoms; wherein said monocyclic 6-membered aromatic ring is substituted with one C₃₋₆cycloalkyl and wherein said monocyclic 6-membered aromatic ring is optionally substituted with one or two additional substituents selected from the group consisting of C₃₋₆cycloalkyl, cyano, and C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 6-membered aromatic ring containing one, two or three nitrogen atoms; wherein said monocyclic 6-membered aromatic ring is substituted with one C₃₋₆cycloalkyl and wherein said monocyclic 6-membered aromatic ring is optionally substituted with one or two additional substituents selected from the group consisting of C₃₋₆cycloalkyl, cyano, and C₁₋₄alkyl; and
R^{1b} represents F.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 6-membered aromatic ring containing one, two or three nitrogen atoms; wherein said monocyclic 6-membered aromatic ring is substituted with one C₃₋₆cycloalkyl.

In an embodiment, the present invention relates to those compounds of Formula (I) and the pharmaceutically acceptable salts, and the solvates thereof, or any subgroup thereof as mentioned in any of the other embodiments, wherein Het represents a monocyclic 6-membered aromatic ring containing one, two or three nitrogen atoms; wherein said monocyclic 6-membered aromatic ring is substituted with one C₃₋₆cycloalkyl; and
R^{1b} represents F.

In an embodiment, the present invention relates to those compounds of Formula (I) and the pharmaceutically acceptable salts, and the solvates thereof, or any subgroup thereof as mentioned in any of the other embodiments, wherein Het represents

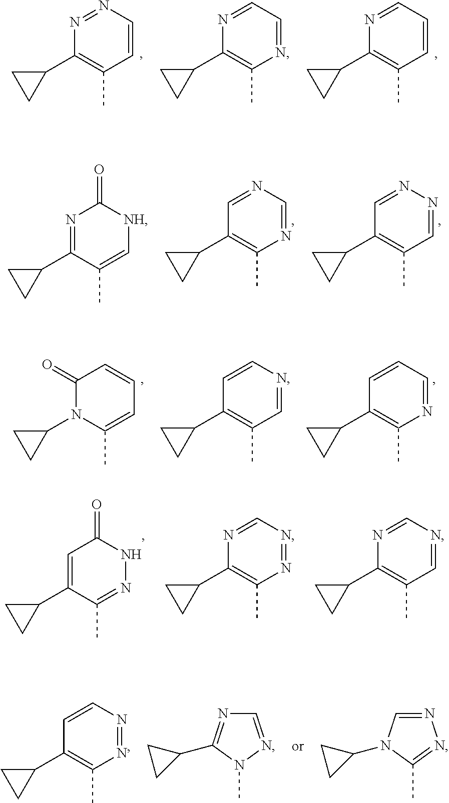

each optionally substituted with one cyano or C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R³ represents Cy².

In an embodiment, the present invention relates to those compounds of Formula (I) and the pharmaceutically acceptable salts, and the solvates thereof, or any subgroup thereof as mentioned in any of the other embodiments, wherein Cy² represents C₃₋₇cycloalkyl; wherein said C₃₋₇cycloalkyl is substituted with one, two, three or four substituents each independently selected from the group consisting of Het^{6a}, Het^{6b}, and —NR^{9a}R^{9b}.

In an embodiment, the present invention relates to those compounds of Formula (I) and the pharmaceutically acceptable salts, and the solvates thereof, or any subgroup thereof as mentioned in any of the other embodiments, wherein Cy² represents C₃₋₇cycloalkyl; wherein said C₃₋₇cycloalkyl is substituted with one or two substituents each independently selected from the group consisting of Het^{6a} and Het^{6b}.

In an embodiment, the present invention relates to those compounds of Formula (I) and the pharmaceutically acceptable salts, and the solvates thereof, or any subgroup thereof as mentioned in any of the other embodiments, wherein R³ represents Cy²; and Cy² represents C₃₋₇cycloalkyl; wherein said C₃₋₇cycloalkyl is substituted with one or two substituents each independently selected from the group consisting of Het^{6a} and Het^{6b}.

In an embodiment, the present invention relates to those compounds of Formula (I) and the pharmaceutically acceptable salts, and the solvates thereof, or any subgroup thereof as mentioned in any of the other embodiments, wherein R³ represents cyclobutyl substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

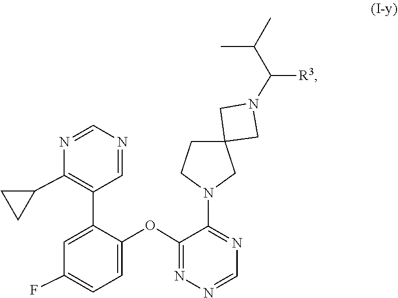

wherein R³ is as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-z):

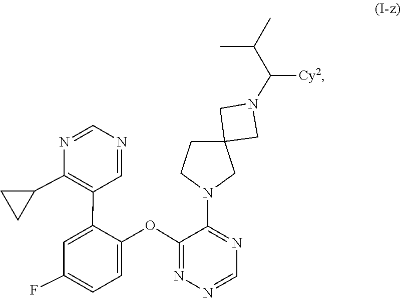

wherein Cy² is as defined for the compounds of Formula (I) or any subgroup thereof as mentioned in any of the other embodiments.

In an embodiment, the present invention concerns novel compounds of Formula (I-z),

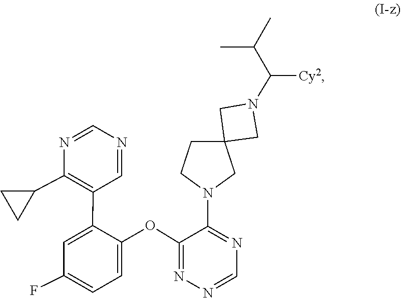

and the tautomers and the stereoisomeric forms thereof, wherein
Cy² represents C₃₋₇cycloalkyl; wherein said C₃₋₇cycloalkyl is substituted on one or two carbon atoms with one substituent selected from the group consisting of Het^{b}a, Het^{6b}, and —NR^{9a}R^{9b};
R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₁₀cycloalkyl; and —S(═O)₂—C₁₋₄alkyl;
Het^{6a} represents a monocyclic N-linked 4- to 7-membered fully saturated heterocyclyl containing one N-atom; wherein said heterocyclyl is optionally substituted on one or two carbon atoms with in total one, two, three or four substituents each independently selected from the group consisting of —O—C₁₋₄alkyl; —O—C₃₋₆cycloalkyl;
—S(═O)₂—C₁₋₄alkyl; and C₁₋₄alkyl;
Het^{6b} represents a bicyclic N-linked 6- to 11-membered fully saturated heterocyclyl containing one N-atom and optionally one additional heteroatom selected from O and N; wherein said heterocyclyl is optionally substituted on one carbon atom with one —O—C₁₋₄alkyl; and in case a second nitrogen atom is present in said heterocyclyl, said second nitrogen atom is substituted with a substituent selected from the group consisting of —C(═O)—C₁₋₄alkyl and C₁₋₄alkyl;
and the pharmaceutically acceptable salts and the solvates thereof.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, and the free bases, any pharmaceutically acceptable salts, and the solvates thereof.

All possible combinations of the above indicated embodiments are considered to be embraced within the scope of the invention.

### Methods for the Preparation of Compounds of Formula (I)

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

### Scheme 1

In general, compounds of Formula (I) wherein Y¹ is limited to Y^{1a} being —O— or —NR^{5c}—, hereby named compounds of Formula (Ia), (Ib), (Ic), (Id), (Ie), can be prepared according to the following reaction Scheme 1. In Scheme 1, W¹ represents fluoro, chloro, bromo or iodo; all other variables are defined according to the scope of the present invention.

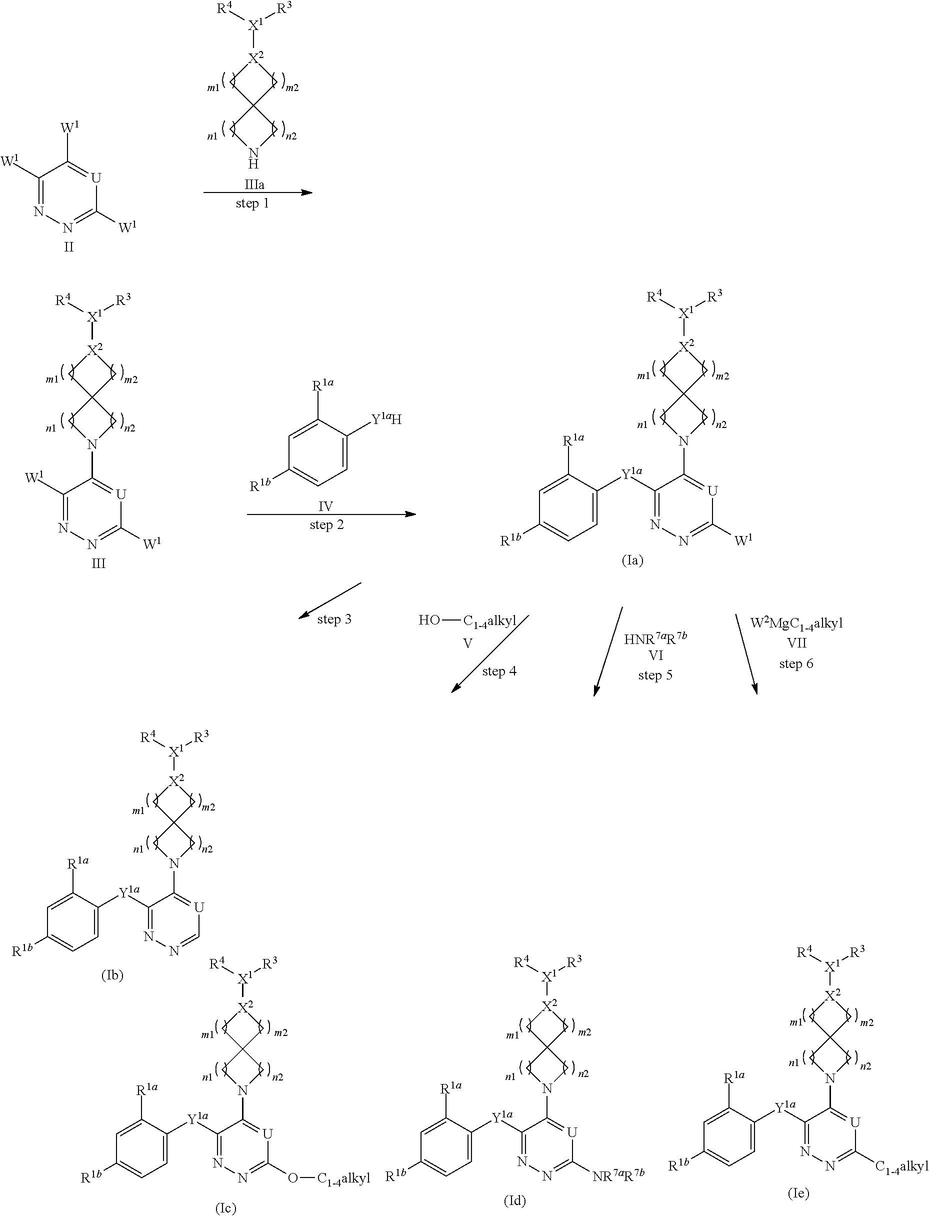

In Scheme 1, the following reaction conditions apply:
Step 1: at a suitable temperature such as ranged from room temperature to 90° C., in the presence of a suitable base such as for example diisopropylethylamine or triethylamine or sodium carbonate, in a suitable solvent such as for example acetonitrile or dimethylformamide or dichloromethane;
Step 2: at a suitable temperature range from room temperature to 130° C., in presence of a suitable base such as for example cesium carbonate, in a suitable solvent such as for example dimethylformamide or 1-methyl-2-pyrrolidinone;
Alternatively, at a suitable temperature such as for example room temperature, in the presence of a suitable deprotonating agent such as for example sodium hydride, in a suitable solvent such as for example dimethylsulfoxide;
Alternatively, at a suitable temperature such as room temperature, in the presence a suitable base such as 1,8-Diazabicyclo[5.4.0]undec-7-ene (DBU), in a suitable solvent such as for example tetrahydrofuran;
Step 3: at a suitable temperature such as room temperature, in the presence of a suitable catalyst such as palladium on charcoal (Pd/C), in a suitable solvent such as methanol, under H₂ pressure such as for example from 1 to 3 bar, optionally in the presence of a base such as triethylamine;
Alternatively, at a suitable temperature such as room temperature, in the presence of a suitable catalyst such as for example 1,1′-Bis(diphenylphosphino)ferrocene-palladium(II)dichloride dichloromethane complex, a suitable reducing agent such sodium borohydride, a suitable base such as for example N,N,N′,N′-tetramethylethylenediamine, in a suitable solvent such as for example tetrahydrofuran;
Step 4: at a suitable temperature range from 100 to 130° C., in presence of a suitable base such as for example cesium carbonate, in a suitable solvent such as for example dimethylformamide or 1-methyl-2-pyrrolidinone;
Step 5: at a suitable temperature range from 100 to 130° C., in presence of a suitable base such as for example cesium carbonate, in a suitable solvent such as for example dimethylformamide or 1-methyl-2-pyrrolidinone;
alternatively, at a suitable temperature ranged from 80 to 100° C., in presence of a suitable catalyst such as palladium acetate (Pd(OAc)₂), in presence of a suitable ligand such as for example 2,2′-bis(diphenylphosphino)-1,1′-binaphthyl, in presence of a suitable base such as cesium carbonate, in a suitable solvent such as for example dioxane;
Step 6: at a suitable temperature from room temperature to 60° C., in presence of a suitable catalyst such as palladium acetate (Pd(OAc)₂) or Tris(dibenzylideneacetone)dipalladium(0) (Pd₂dba₃), in presence or not of a suitable ligand such as for example triphenylphosphine, in a suitable solvent such as for example dioxane;

### Scheme 2

In general, compounds of Formula (I) wherein Y¹ is limited to —CH₂—, and R² is limited to W¹, hereby named compounds of Formula (If), can be prepared according to the following reaction Scheme 2. In Scheme 2, all other variables are defined according to the scope of the present invention.

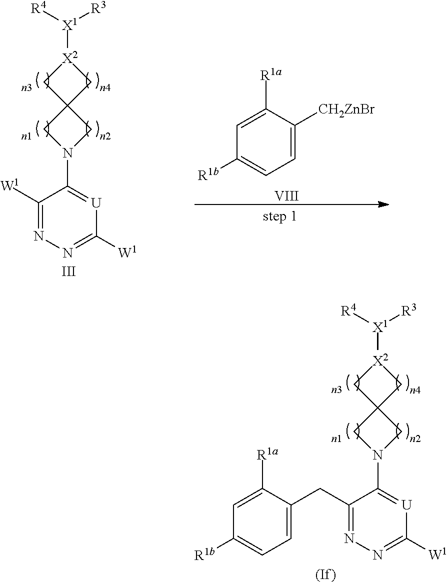

In Scheme 2, the following reaction conditions apply:
Step 1: at a suitable temperature ranged from 60° C. to 100° C., in presence of a suitable catalyst such as palladium acetate (Pd(OAc)₂) or Tris(dibenzylideneacetone)dipalladium(0) (Pd₂(dba)₃) or Tetrakis(triphenylphosphine)palladium(0), in a suitable solvent such as for example tetrahydrofuran or dioxane.

The skilled person will realize that starting from compound (If), analogous chemistry as reported in steps 3, 4, 5 and 6 in scheme 1 could be performed.

### Scheme 3

In general, compounds of Formula (I) wherein Y¹ is limited to —CR^{5a}R^{5b}— and R² is limited to W¹, hereby named compounds of Formula (Ig), can be prepared according to the following reaction Scheme 3. In Scheme 3 at least one of R^{5a} and R^{5b} is other than hydrogen. All other variables are defined according to the scope of the present invention.

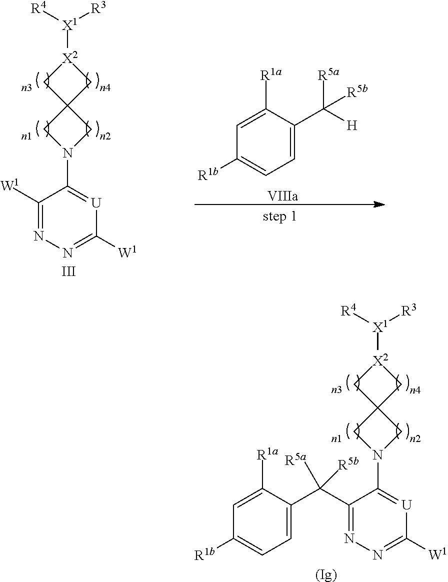

In Scheme 3, the following reaction condition apply:
Step 1: at a suitable temperature ranged from 80° C. to 200° C., in presence of a suitable catalyst such as palladium acetate (Pd(OAc)₂), in the presence of a suitable ligand such as for example triphenylphosphine or tricyclohexylphosphine, in a suitable solvent such as for example dioxane, preferably in sealed conditions, optionally under microwave irradiation.

The skilled person will realize that starting from compound (Ig), analogous chemistry as reported in steps 3, 4, 5 and 6 in scheme 1 could be performed.

### Scheme 4

In general, compounds of Formula (I) hereby named compounds of Formula (Ib) can be alternatively prepared according to the following reaction Scheme 4. In Scheme 4, PG¹ represents a suitable protecting group, such as for example tert-butyloxycarbonyl and LG¹ is a leaving group such as for example chloro, bromo, iodo or tosylate or mesylate; all other variables are defined as listed before or according to the scope of the present invention.

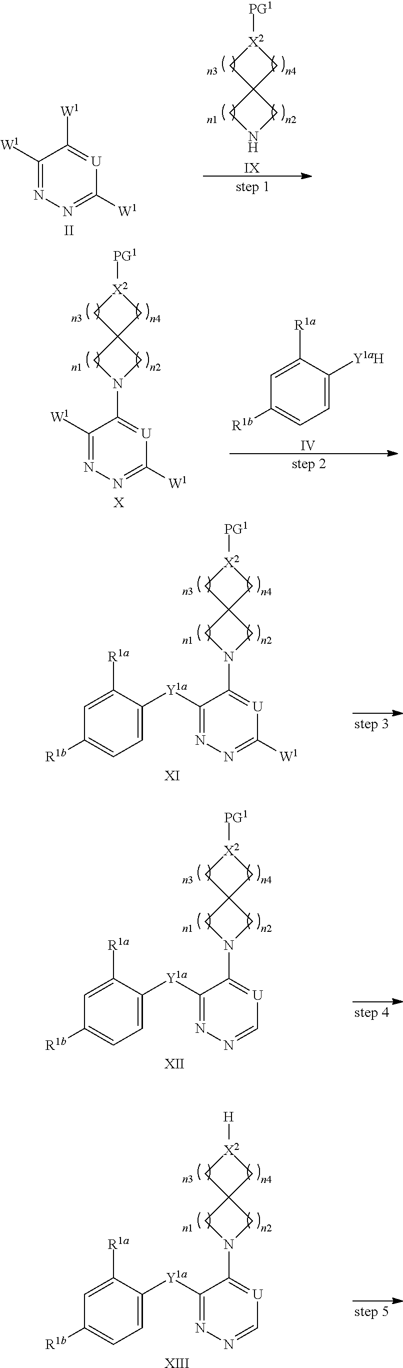

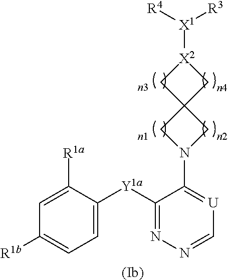

In Scheme 4, the following reaction conditions apply:
Step 1: at a suitable temperature such as ranged from room temperature to 90° C., in the presence of a suitable base such as for example diisopropylethylamine or triethylamine or sodium carbonate, in a suitable solvent such as for example acetonitrile or dimethylformamide or dichloromethane;
Step 2: at a suitable temperature range from room temperature to 130° C., in presence of a suitable base such as for example cesium carbonate, in a suitable solvent such as for example dimethylformamide or 1-methyl-2-pyrrolidinone;
Alternatively, at a suitable temperature such as for example room temperature, in the presence of a suitable deprotonating agent such as for example sodium hydride, in a suitable solvent such as for example dimethylsulfoxide;
Alternatively, at a suitable temperature such as room temperature, in the presence a suitable base such as 1,8-Diazabicyclo[5.4.0]undec-7-ene (DBU), in a suitable solvent such as for example tetrahydrofuran;
Step 3: at a suitable temperature such as room temperature, in the presence of a suitable catalyst such as palladium on charcoal (Pd/C), in a suitable solvent such as methanol, under H₂ pressure such as for example from 1 to 3 bar;
Alternatively, at a suitable temperature such as room temperature, in the presence of a suitable catalyst such as for example 1,1′-Bis(diphenylphosphino)ferrocene-palladium(II)dichloride dichloromethane complex, a suitable reducing agent such sodium borohydride, a suitable base such as for example N,N,N′,N′-tetramethylethylenediamine, in a suitable solvent such as for example tetrahydrofuran;
Step 4: when PG¹ is tert-butyloxycarbonyl, at a suitable temperature range such as for example from 0° C. to room temperature, in the presence of suitable cleavage conditions, such as for example an acid such as HCl or trifluoroacetic acid in a suitable solvent such as acetonitrile or DCM or methanol (MeOH);
Step 5: represents all type of reactions, such as for examples reductive amination, nucleophilic substitution, leading to final examples (Ib);

The skilled person will realize that starting from intermediate XI, analogous chemistry as reported in steps 3, 4, 5 and 6 in scheme 1 could be performed.

### Scheme 5

In general, compounds of Formula (I) wherein U is limited to N and Y¹ is limited to Y^{1b} being O, hereby named compounds of Formula (Iba) can be prepared according to the following reaction Scheme 5. In Scheme 5, PG¹ represents a suitable protecting group, such as for example tert-butyloxycarbonyl and W² a leaving group such as for example chloro, tosylate or mesylate; all other variables are defined according to the scope of the present invention.

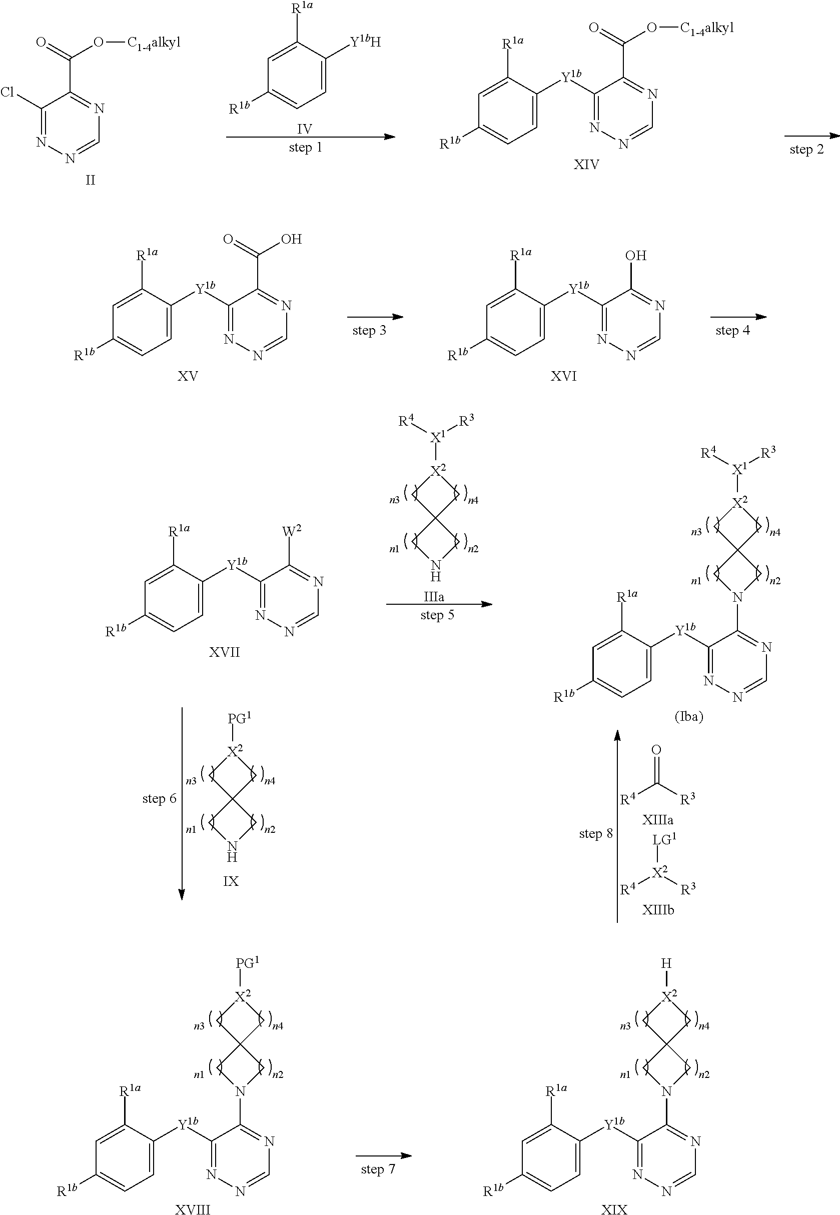

In Scheme 5, the following reaction conditions apply:
Step 1: at a suitable temperature such as room temperature, in the presence of a suitable base such as for example potassium carbonate, in a suitable solvent such as for example dimethylformamide;
Step 2: at a suitable temperature such as room temperature, in presence of a suitable base such as lithium hydroxide, in a suitable solvent such as for example a mixture of tetrahydrofurane, ethanol and water;
Step 3: at a suitable temperature such as room temperature, in the presence of a dibromoisocyanurate, in a suitable solvent such as dichloroethane;
Step 4: when W² is chloro, at a suitable temperature range such as room temperature, in the presence of a chlorinating reagent such as oxalyl chlorine, in the presence of a catalytic amount of dimethylformamide, in the presence of a suitable base such as triethylamine, in a suitable solvent such as dichloromethane;
When W² is a trifluoroethoxy, at a suitable temperature such as 65° C., in the presence of 2,2,2-trifluoroethanol as solvent or not, suitable activating agents such as 1,3-dibromo-1,3,5-triazinane-2,4,6-trione, in the presence of molecular sieve;
Step 5: At a suitable temperature such as room temperature, in the presence of a suitable base such as for example triethylamine or 1,8-Diazabicyclo[5.4.0]undec-7-ene (DBU), in a suitable solvent such as for example dichloromethane or acetonitrile;
Step 6: At a suitable temperature such as room temperature, in the presence of a suitable base such as for example triethylamine or 1,8-Diazabicyclo[5.4.0]undec-7-ene (DBU), in a suitable solvent such as for example dichloromethane or acetonitrile;
Step 7: when PG¹ is tert-butyloxycarbonyl, at a suitable temperature range such as for example from 0° C. to room temperature, in the presence of suitable cleavage conditions, such as for example an acid such as HCl or trifluoroacetic acid in a suitable solvent such as acetonitrile or DCM or methanol (MeOH);
Step 8: represents all type of reactions, such as for examples reductive amination, nucleophilic substitution leading to final examples (Iba).

### Scheme 6

In general, intermediates of formula IIIa can be prepared according to the following reaction Scheme 5. In Scheme 5, PG² represents a suitable protecting group, such as for example benzyloxycarbonyl; all other variables are defined according to the scope of the present invention or as defined in the previous schemes.

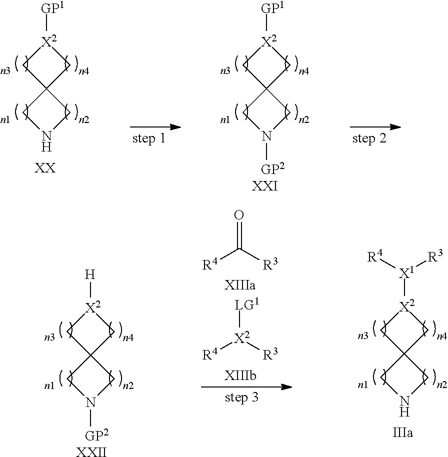

Step 1: at a suitable temperature such as room temperature, in the presence of benzyl chloroformate, in the presence of a suitable base such as for example triethylamine, in a suitable solvent such as for example dichloromethane;
Step 2: when PG¹ is tert-butyloxycarbonyl, at a suitable temperature range such as for example from 0° C. to room temperature, in the presence of suitable cleavage conditions, such as for example an acid such as HCl or trifluoroacetic acid in a suitable solvent such as acetonitrile or DCM or methanol (MeOH);
Step 3: represents all type of reactions, such as for examples reductive amination, nucleophilic substitution leading to intermediate IIIa.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

### Pharmacology

It has been found that the compounds of the present invention block the interaction of menin with MLL proteins and oncogenic MLL fusion proteins. Therefore the compounds according to the present invention and the pharmaceutical compositions comprising such compounds may be useful for the treatment or prevention, in particular treatment, of diseases such as cancer, myelodysplastic syndrome (MDS) and diabetes.

In particular, the compounds according to the present invention and the pharmaceutical compositions thereof may be useful in the treatment or prevention of cancer. According to one embodiment, cancers that may benefit from a treatment with menin/MLL inhibitors of the invention comprise leukemias, myeloma or a solid tumor cancer (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

Hence, the invention relates to compounds of Formula (I), the tautomers and the stereoisomeric forms thereof, and the pharmaceutically acceptable salts, and the solvates thereof, for use as a medicament.

The invention also relates to the use of a compound of Formula (I), a tautomer or a stereoisomeric form thereof, or a pharmaceutically acceptable salt, or a solvate thereof, or a pharmaceutical composition according to the invention, for the manufacture of a medicament.

The present invention also relates to a compound of Formula (I), a tautomer or a stereoisomeric form thereof, or a pharmaceutically acceptable salt, or a solvate thereof, or a pharmaceutical composition according to the invention, for use in the treatment, prevention, amelioration, control or reduction of the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

Also, the present invention relates to the use of a compound of Formula (I), a tautomer or a stereoisomeric form thereof, or a pharmaceutically acceptable salt, or a solvate thereof, or a pharmaceutical composition according to the invention, for the manufacture of a medicament for treating, preventing, ameliorating, controlling or reducing the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

The invention also relates to a compound of Formula (I), a tautomer or a stereoisomeric form thereof, or a pharmaceutically acceptable salt, or a solvate thereof, for use in the treatment or prevention of any one of the diseases mentioned hereinbefore.

The invention also relates to a compound of Formula (I), a tautomer or a stereoisomeric form thereof, or a pharmaceutically acceptable salt, or a solvate thereof, for use in treating or preventing any one of the diseases mentioned hereinbefore.

The invention also relates to the use of a compound of Formula (I), a tautomer or a stereoisomeric form thereof, or a pharmaceutically acceptable salt, or a solvate thereof, for the manufacture of a medicament for the treatment or prevention of any one of the disease conditions mentioned hereinbefore.

The compounds of the present invention can be administered to mammals, preferably humans, for the treatment or prevention of any one of the diseases mentioned hereinbefore.

In view of the utility of the compounds of Formula (I), the tautomers and the stereoisomeric forms thereof, and the pharmaceutically acceptable salts, and the solvates thereof, there is provided a method of treating warm-blooded animals, including humans, suffering from any one of the diseases mentioned hereinbefore.

Said method comprises the administration, i.e. the systemic or topical administration, of a therapeutically effective amount of a compound of Formula (I), a tautomer or a stereoisomeric form thereof, or a pharmaceutically acceptable salt, or a solvate thereof, to warm-blooded animals, including humans.

Therefore, the invention also relates to a method for the treatment or prevention of any one of the diseases mentioned hereinbefore comprising administering a therapeutically effective amount of compound according to the invention to a patient in need thereof.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to administration.

The present invention also provides compositions for preventing or treating the disorders referred to herein. Said compositions comprising a therapeutically effective amount of a compound of Formula (I), a tautomer or a stereoisomeric form thereof, or a pharmaceutically acceptable salt, or a solvate thereof, and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions may be prepared by any methods well known in the art of pharmacy, for example, using methods such as those described in Gennaro et al. Remington's Pharmaceutical Sciences (18^{th} ed., Mack Publishing Company, 1990, see especially Part 8 Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular condition, in particular tumour, being treated and the particular host being treated.

The following examples further illustrate the present invention.

### EXAMPLES

Several methods for preparing the compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| ACN | acetonitrile |
| AcOH | acetic acid |
| aq. | aqueous |
| Ar | argon |
| Boc | tert-butyloxycarbonyl |
| DCE | dichloroethane |
| DCM | dichloromethane |
| DIC | N,N′-Diisopropylcarbodiimide |
| DIEA or DIPEA | N,N-diisopropylethylamine |
| DMAP | 4-dimethylaminopyridine |
| DMF | N,N-dimethylformamide |
| dppf | 1,1′-ferrocenediyl-bis(diphenylphosphine) |
| ee | enantiomeric excess |
| ESI | electrospray ionization |
| EtOAc or EA | ethyl acetate |
| EDCI | N′-(ethylcarbonimidoyl)-N,N-dimethyl-1,3- |
| | propanediamine monohydrochloride |
| EtOH | ethanol |
| h | hour(s) |
| min | minute(s) |
| HATU | 1-[bis(dimethylamino)methylene]-1H-1,2,3- |
| | triazolo[4,5-b]pyridinium 3-oxid |
| | hexafluorophosphate |
| HPLC | high performance liquid chromatography |
| Prep-HPLC | Preparative HPLC |
| Prep CC | Preparative column chromatography |
| MeOH | methanol |
| MeTHF | 2-methyltetrahydrofuran |
| NaBH(OAc)₃ | sodium triacetoxyborohydride |
| NaBH₃CN | sodium cyanoborohydride |
| Pd(dppf)Cl₂ | [1,1′-bis(diphenyl- |
| | phosphino)ferrocene]dichloropalladium(II) |
| Pd(dppf)Cl₂•DCM | [1,1′-bis(diphenylphosphino)ferrocene]- |
| | dichloropalladium(II) complex with |
| | dichloromethane |
| PE | petroleum ether |
| RT or r.t. | room temperature |
| SFC | super critical fluid chromatography |
| TEA or Et₃N | triethylamine |
| TFA | trifluoroacetic acid |
| THF | tetrahydrofuran |
| TLC | thin layer chromatography |
| Celite ® | diatomaceous earth |
| PyBroP | bromotripyrrolidinophosphonium |
| | hexafluorophosphate |
| Cbz | benzyloxycarbonyl |
| FCC | flash column chromatography |
| HOBt | 1-hydroxy-1H-benzotriazole |
| Rf | retention factor |
| FA | formic acid |
| T₃P | propylphosphonic anhydride |
| i-PrMgCl | isopropylmagnesium chloride |
| i-PrOH or IPA | 2-propanol |
| iPrNH₂ | isopropylamine |
| ‘sat.’ or ‘Sat.’ | saturated |
| Psi | pounds per square inch |
| THP | tetrahydropyran |
| Dess-Martin | 1,1,1-tris(acetyloxy)-1,1-dihydro-1,2- |
| periodinane | benziodoxol-3-(1H)-one |
| Et₂O | diethylether |
| Na₂SO₄ | sodium sulfate |
| MgSO₄ | magnesium sulfate |
| N₂ | nitrogen |
| NH₄Cl | ammonium chloride |
| NaHCO₃ | sodium hydrogenocarbonate |
| Na₂CO₃ | sodium carbonate |
| K₂CO₃ | potassium carbonate |
| NaOH | sodium hydroxide |
| LiOH•H₂O | lithium hydroxide monohydrate |
| EtOH | ethanol |
| LiAlH₄ | lithium aluminium hydride |
| HCl | hydrochloric acid |
| ZnCl₂ | zinc chloride |
| Na₂SO₃ | sodium sulfite |
| NH₃•H₂O | ammonium hydroxide |
| v/v | volume to volume |
| Pd(amphos)Cl₂ | Bis(di-tert-butyl(4-dimethyl- |
| | aminophenyl)phosphine)dichloropalladium(II) |
| SPhos Pd G2 | Chloro(2-dicyclohexylphosphino-2′,6′- |
| | dimethoxy-1,1′-biphenyl)[2-(2′-amino-1,1′- |
| | biphenyl)]palladium(II) |
| DBU | 1,8-Diazabicyclo[5.4.0]undec-7-ene |
| TBAF | Tetra-n-butylammonium fluoride |
| TBACN | Tetrabutylammonium cyanide |
| TsCl | p-Toluenesulfonyl chloride |
| TsOH | p-Toluenesulfonic acid |
| DABCO | 1,4-diazabicyclo[2,2,2]octane |
| KF | potassium fluoride |
| (NH₄)₂S₂O₈ | Ammonium peroxydisulfate |
| AgNO₃ | Silver nitrate |
| NaBH₄ | Sodium borohydride |
| TMEDA | N,N,N′,N′-tetramethylethylenediamine |
| EA | Ethyl acetate |
| Catacxium A-Pd- | Mesylate[(di(1-adamantyl)-n-butylphosphine)-2- |
| G3 | (2′-amino-1,1′-biphenyl)]palladium(II), |
| | [(Di(1-adamanty])-butylphosphine)-2-(2′-amino- |
| | 1,1′-biphenyl)]palladium(II) methanesulfonate |
| PPh₃ | triphenylphosphine |
| Pd(PPh₃)₄ | Tetrakis(triphenylphosphine)palladium(0) |
| Cs₂CO₃ | Cesium Carbonate |
| POCl₃ | phosphorus trichloride |
| LCMS | Liquid Chromatography Mass Spectometry |
| TMSI | Iodotrimethylsilane |
| Brettphos | 2-(Dicyclohexylphosphino)3,6-dimethoxy-2′,4′,6′- |
| | triisopropyl-1,1′-biphenyl |
| Brettphos-Pd-G3 | [(2-Di-cyclohexylphosphino-3,6-dimethoxy-2′,4′,6′- |
| | triisopropyl-1,1′-biphenyl)-2-(2′-amino-1,1′ - |
| | biphenyl)]palladium(II) methanesulfonate |
| | methanesulfonate |
| MTBE | ethyl tert-butyl ether |
| TBDPS | tert-Butyldiphenylsilyl |
| MeMgBr | Methyl magnesium bromide |
| MS | Mass Spectometry |
| hex | n-hexane |
| Josiphos SL-J009-1 | {(R)-1-[(Sp)-2- |
| Pd G3 | (Dicyclohexylphosphino)ferrocenyl]ethyldi-tert- |
| | butylphosphine}[2-(2′-amino-1,1′- |
| | biphenyl)]palladium(II) methanesulfonate |
| DMA | dimethylacetamide |
| tBu XPhos or | 2-Di-tert-butylphosphino-2′,4′,6′- |
| t-Bu XPhos | triisopropylbiphenyl |
| tBu XPhos-Pd-G3, | [(2-Di-tert-butylphosphino-2′,4′,6′-triisopropyl-1,1′- |
| t-BuXPhos-Pd-G3, | biphenyl)-2-(2′-amino-1,1′-biphenyl)] palladium(II) |
| t-BuXPhos Pd G3 | methanesulfonate |
| or tBuXPhos Pd G3 | |
| NMP | N-Methyl-2-pyrrolidone |
| t-BuONa | Sodium tert-butoxide |
| n-BuLi | n-butyllithium |
| [Ph₃P]Pd | Tetrakis(triphenylphosphine)palladium(0) |

As understood by a person skilled in the art, compounds synthesized using the protocols as indicated may exist as a solvate e.g. hydrate, and/or contain residual solvent or minor impurities. Compounds isolated as a salt form, may be integer stoichiometric i.e. mono- or di-salts, or of intermediate stoichiometry. When an intermediate or compound in the experimental part below is indicated as ‘HCl salt’ without indication of the number of equivalents of HCl, this means that the number of equivalents of HCl was not determined.

The stereochemical configuration for centers in some compounds may be designated “R” or “S” when the mixture(s) was separated; for some compounds, the stereochemical configuration at indicated centers has been designated as “*R” or “*S” when the absolute stereochemistry is undetermined (even if the bonds are drawn stereo specifically) although the compound itself has been isolated as a single stereoisomer and is enantiomerically pure.

For example, it will be clear that Compound 5

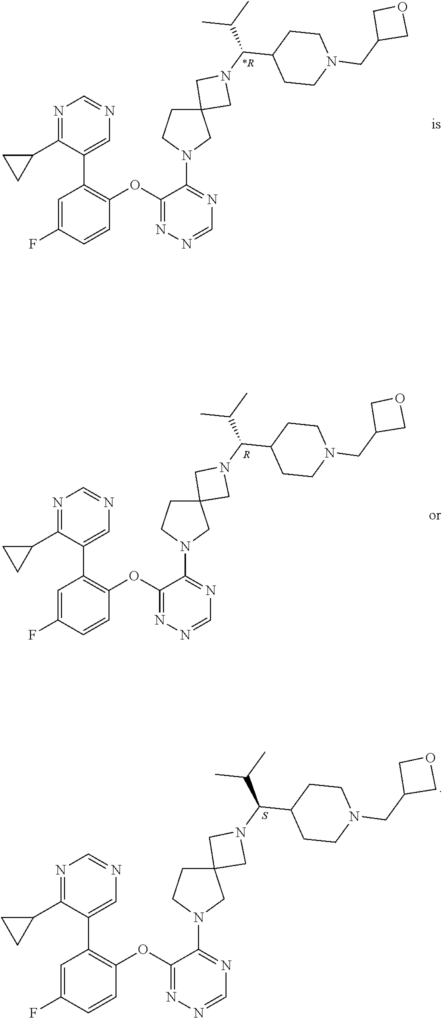

For example, it will be clear that Compound 14

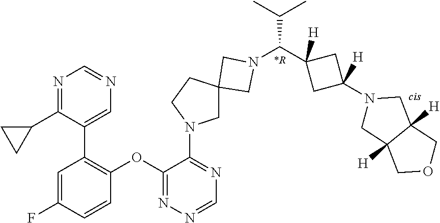 [SMILES: CC(C)[C@H]([C@H]1C[C@@H](N2C[C@H]3COC[C@H]3C2)C1)N1CC2(CCN(c3ncnnc3Oc3ccc(F)cc3-c3cncnc3C3CC3)C2)C1]

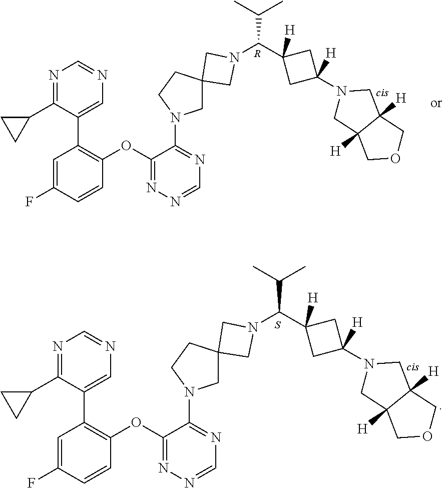

For compounds such as for example 214 and 215, wherein the stereochemical configuration of two stereocenters is indicated by * (e.g. *R or *S), the absolute stereochemistry of the stereocenters is undetermined (even if the bonds are drawn stereospecifically), although the compound itself has been isolated as a single stereoisomer and is enantiomerically pure. In this case, the configuration of the first stereocenter is independent of the configuration of the second stereocenter in the same compound.

For example, for Compound 214:

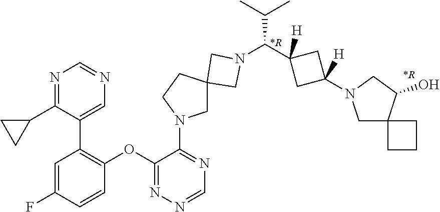 [SMILES: CC(C)[C@H]([C@H]1C[C@@H](N2C[C@H](O)C3(CCC3)C2)C1)N1CC2(CCN(c3ncnnc3Oc3ccc(F)cc3-c3cncnc3C3CC3)C2)C1]

this means that the compound is

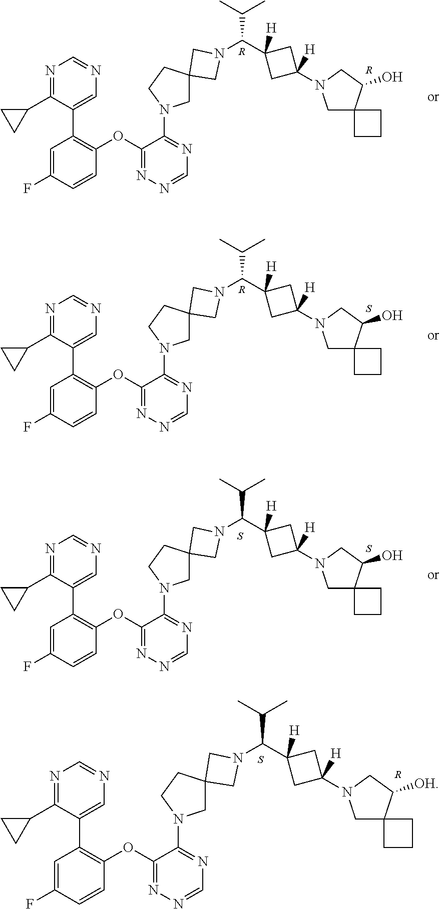

The paragraphs above about stereochemical configurations, also apply to intermediates.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

When a stereocenter is indicated with ‘RS’ this means that a racemic mixture was obtained at the indicated centre, unless otherwise indicated.

A skilled person will understand that when Intermediates or Compounds are reported in Tables, the synthetic methodology from the indicated starting material to desired Intermediate/Compound might go over one or more reaction steps.

When two enantiomers, diastereomers or isomers are present in the same cell of one of the tables below (e.g. Compound 1a and Compound 1b), a skilled person will understand that these Intermediates or Compounds were separated from each other by using a suitable chromatographic method e.g. SFC or reversed phase separation.

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Example A1

### Preparation of Intermediate 2

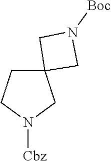 [SMILES: CC(C)(C)OC(=O)N1CC2(CCN(C(=O)OCc3ccccc3)C2)C1]

Benzyl chloroformate (6.03 g, 35.3 mmol) was added to a 0° C. (ice/water) mixture of tert-butyl 2,6-diazaspiro[3.4]octane-2-carboxylate (5.00 g, 23.6 mmol), TEA (16.5 mL, 117 mmol) and CH₂Cl₂ (50 mL). Then, DMAP (57.5 mg, 0.471 mmol) was added into the above mixture. The reaction mixture was stirred at 25° C. for 12 hours. The reaction mixture was concentrated to dryness under reduced pressure to give the crude product, which was purified by FCC (eluent: petroleum ether:ethyl acetate=100:1 to 2:1) to yield intermediate 2 (7.00 g, 83.7% yield) as a yellow oil.

### Preparation of Intermediate 3

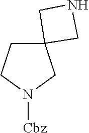 [SMILES: O=C(OCc1ccccc1)N1CCC2(CNC2)C1]

To a solution of intermediate 2 (25.0 g, 72.2 mmol) in dry dichloromethane (15 mL) was added trifluoroacetic acid (30 mL). The reaction mixture was stirred at 25° C. for 30 min. The reaction mixture was concentrated under reduced pressure to give a residue, which was suspended into aqueous NaOH (4 g in H₂O (40 mL)) and extracted with dichloromethane (20 mL×3). The combined organic layers were dried over anhydrous Na₂SO₄, filtered and concentrated under reduced pressure to yield intermediate 3 (16.0 g) as a yellow oil.

### Example A2

### Preparation of Intermediate 5

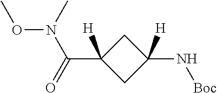 [SMILES: CON(C)C(=O)[C@H]1C[C@@H](NC(=O)OC(C)(C)C)C1]

To a solution of cis-3-[[(1,1-dimethylethoxy) carbonyl]amino]-cyclobutanecarboxylic acid (10.0 g, 46.5 mmol) in DMF (100 mL) was added HOBt (8.15 g, 60.3 mmol), EDCI (11.6 g, 60.5 mmol) and DIEA (30.0 mL, 182 mmol, 0.782 g/mL) at 0° C. Then N,O-dimethylhydroxylamine hydrochloride (5.90 g, 60.5 mmol) was added at 0° C. The mixture was stirred at room temperature for 16 hours. The mixture was diluted with ethyl acetate (500 mL). The mixture was washed with 1 M HCl (150 mL), saturated NaHCO₃ (100 mL×2) and brine (300 mL×3), dried over Na₂SO₄, filtered and concentrated under reduced pressure to give intermediate 5 (11.0 g, crude) as a white solid, which was used in the next step without further purification.

### Preparation of Intermediate 6

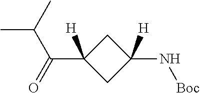 [SMILES: CC(C)C(=O)[C@H]1C[C@@H](NC(=O)OC(C)(C)C)C1]

To a solution of intermediate 5 (11.0 g, 6.97 mmol) in THE (100 mL) was added isopropylmagnesium chloride (64.0 mL, 128 mmol, 2 M in THF) dropwise at 0° C. under N₂ atmosphere. The mixture was stirred at room temperature for 12 hours under N₂ atmosphere. The mixture was quenched with saturated NH₄Cl (100 mL). The mixture was filtered through a pad of Celite® and the filtrate was concentrated under reduced pressure. The mixture was extracted with ethyl acetate (200 mL×2). The combined organic layers were washed with brine (200 mL×2), dried over Na₂SO₄, filtered and concentrated under reduced pressure. The crude product was purified by flash column chromatography over silica gel (eluent: petroleum ether:ethyl acetate from 1:0 to 5:1, TLC: petroleum ether:ethyl acetate=5:1, Rf=0.4) to yield intermediate 6 (6.30 g) as a white solid.

### Preparation of Intermediate 7

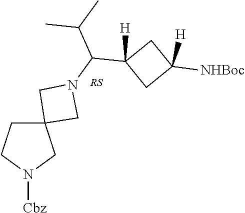 [SMILES: CC(C)C([C@H]1C[C@@H](NC(=O)OC(C)(C)C)C1)N1CC2(CCN(C(=O)OCc3ccccc3)C2)C1]

To a solution of intermediate 3 (2.80 g, 11.4 mmol) and intermediate 6 (3.00 g, 12.4 mmol) in MeOH (50 mL) was added acetic acid (1.50 g, 24.6 mmol). The mixture was stirred at 45° C. for 0.5 hrs. Then sodium cyanotrihydroborate (1.54 g, 24.5 mmol) was added. The mixture was stirred at 45° C. for 12 hrs. The mixture was cooled to room temperature and concentrated under reduced pressure. The residue was diluted with DCM (100 mL). The mixture was washed with saturated NaHCO₃ (50 mL×2) and brine (50 mL×2), dried over Na₂SO₄, filtered and concentrated under reduced pressure. The crude product was purified by flash column chromatography over silica gel (eluent: dichloromethane:Methanol from 1:0 to 10:1, TLC: dichloromethane:Methanol=10:1, Rf=0.5) to yield intermediate 7 (3.40 g, 53.6% purity as measured by LCMS) as a colorless oil.

### Preparation of Intermediate 293

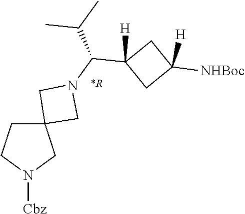 [SMILES: CC(C)[C@H]([C@H]1C[C@@H](NC(=O)OC(C)(C)C)C1)N1CC2(CCN(C(=O)OCc3ccccc3)C2)C1]

Intermediate 7 (10.0 g, 21.2 mmol) was separated by SFC (column: DAICEL CHIRALCEL OD (250 mm*50 mm, 10 um), eluent: 25% (v/v) super critical CO₂ in 0.1% NH₃H₂O EtOH, flow rate: 200 mL/min) to yield intermediate 293 (3.80 g, 38% yield) as a yellow oil.

### Preparation of Intermediate 8

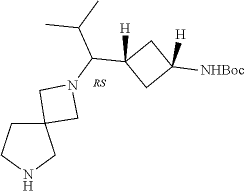 [SMILES: CC(C)C([C@H]1C[C@@H](NC(=O)OC(C)(C)C)C1)N1CC2(CCNC2)C1]

To a solution of intermediate 7 (1.00 g, 2.12 mmol, 53.6% purity) in MeOH (50 mL) was added 1,1,2-trichloroethane (424 mg, 3.18 mmol) and Pd/C (500 mg, w/w %=10% Pd loading). The mixture was stirred under H₂ atmosphere (50 psi) at 40° C. for 4 hrs. The reaction mixture was filtered through a pad of Celite® and the filtrate was concentrated under reduced pressure. The crude product was purified by flash column chromatography over silica gel (eluent: dichloromethane:methanol (0.5% NH₃·H₂O) from 1:0 to 3:1, TLC: dichloromethane:methanol (0.5% NH₃·H₂O)=3:1, Rf=0.4) to yield intermediate 8 (380 mg, 99.1% yield) as a white solid.

### Example A3

### Preparation of Intermediate 10

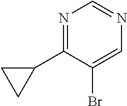

To a solution of 5-bromopyrimidine (30 g, 189 mmol) in 1000 mL of THE was added cyclopropylmagnesium bromide (396 mL, 198 mmol, 0.5 M) at 0° C. under N₂ atmosphere. After addition, the reaction mixture was stirred at room temperature for 4 hours, 4,5-dichloro-3,6-dioxocyclohexa-1,4-diene-1,2-dicarbonitrile (42.8 g, 189 mmol) in 500 mL of THE was added drop wise into the reaction mixture at 0° C. After addition, the reaction mixture was stirred at room temperature for 16 hours. The reaction mixture was concentrated in vacuum and the residue was diluted with 200 mL of EtOAc and 200 mL of water, then separated and the aqueous layer was extracted with EtOAc (200 mL×3), the combined extracts were washed with 1N NaOH (200 mL×2), brine (200 mL), dried over Na₂SO₄, filtered and concentrated in vacuum, the residue was purified by column chromatography (PE/EtOAc from 100/0 to 85/15) to yield intermediate 10 (21.4 g, 55% yield) as a white solid.

The intermediate reported below was prepared following an analogous methodology as described for intermediate 10 starting from the corresponding starting material:

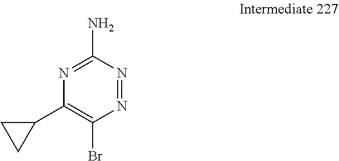 [SMILES: Nc1nnc(Br)c(C2CC2)n1]

### Preparation of Intermediate 11

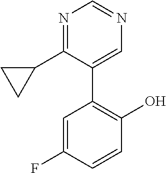 [SMILES: Oc1ccc(F)cc1-c1cncnc1C1CC1]

A mixture of intermediate 10 (16.4 g, 82.4 mmol), (5-fluoro-2-hydroxyphenyl)boronic acid (16.1 g, 103 mmol), Pd(dppf)Cl₂ (3.56 g, 4.86 mmol) and Na₂CO₃ (2 M in water, 82.6 mL, 165 mmol) in dioxane (600 mL) was heated at 90° C. for 3 hours. The above reaction mixture was combined with another batch (prepared starting from 15 g of intermediate 10) for workup and purification. The combined solution was filtered through a pad of Celite® and the filtrate was concentrated in vacuum. The residue was diluted with 200 mL of EtOAc and 200 mL of water, then separated and the aqueous layer was extracted with EtOAc (200 mL×3). The combined extracts were washed with brine (500 mL), dried over Na₂SO₄, filtered and concentrated in vacuum until 100 mL left and filtered to yield intermediate 11 (20.0 g) as brown solid. The filtrate was concentrated and the residue was purified by column chromatography (PE/EtOAc from 100/0 to 50/50) to yield intermediate 11 (10 g) as a brown solid. In total: 30.0 g of intermediate 11 (84% yield).

The intermediate reported below was prepared following an analogous methodology as described for intermediate 11 starting from the corresponding intermediate:

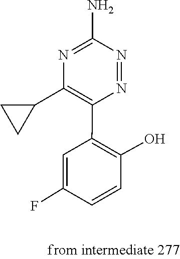 [SMILES: Nc1nnc(-c2cc(F)ccc2O)c(C2CC2)n1]

### Preparation of Intermediate 12

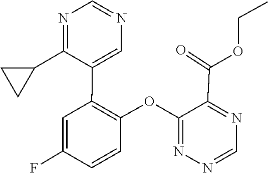 [SMILES: CCOC(=O)c1ncnnc1Oc1ccc(F)cc1-c1cncnc1C1CC1]

K₂CO₃ (9.27 g, 67.1 mmol) was added to a solution of intermediate 11 (5.15 g, 22.4 mmol), ethyl 6-chloro-1,2,4-triazine-5-carboxylate (5.60 g, 29.9 mmol) in DMF (50 mL). The reaction mixture was stirred at room temperature for 1 hour. The mixture was diluted with ethyl acetate (80 mL) and washed with H₂O (40 mL×2) and brine (40 mL). The organic layer was dried over anhydrous Na₂SO₄, filtered and concentrated under reduced pressure to give crude product, which was purified by FCC (eluting with petroleum ether:ethyl acetate=100:0 to 1:1) to yield intermediate 12 (7.00 g, yield 59.1%) as a white solid.

The intermediates reported below were prepared following an analogous methodology as described for intermediate 12 starting from the corresponding intermediates:

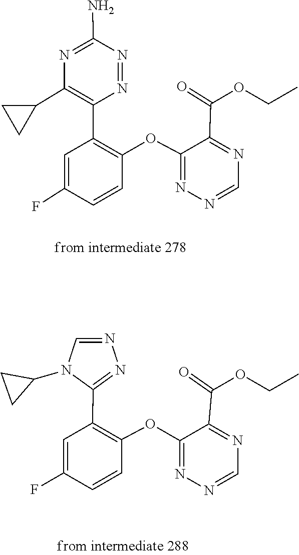 [SMILES: CCOC(=O)c1ncnnc1Oc1ccc(F)cc1-c1nnc(N)nc1C1CC1 ; CCOC(=O)c1ncnnc1Oc1ccc(F)cc1-c1nncn1C1CC1]

### Preparation of Intermediate 13

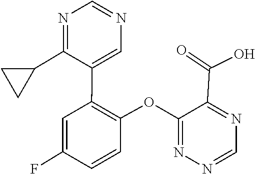 [SMILES: O=C(O)c1ncnnc1Oc1ccc(F)cc1-c1cncnc1C1CC1]

LiOH·H₂O (3.85 g, 91.7 mmol) was added to a solution of intermediate 12 (7.00 g, 18.3 mmol) in THE (50 mL), H₂O (10 mL) and EtOH (5 mL). The mixture was stirred at 25° C. for 2 h. The resultant solution was acidified with 0.5 M HCl to pH=5-6, and extracted with ethyl acetate (10 mL). The aqueous phase was purified by preparative high performance liquid chromatography over Phenomenex Gemini-NX 150*30 mm*5 μm (eluent: (water (0.225% FA):ACN) from 95:5 to 65:35 v/v). The pure fractions were collected and the volatiles were removed under vacuum. The residue was lyophilized to remove the solvent residue completely yielding intermediate 13 (3.85 g, yield 59.4%) as a white solid.

### Alternative Preparation of Intermediate 13

A solution of intermediate 12 (1.80 g, crude) in THE (30 mL) was added to a solution of LiOH H₂O (300 mg, 7.15 mmol) in H₂O (10 mL). The mixture was stirred at r.t. for 2 hours. The reaction mixture was adjusted with 1 N HCl to pH=3-4 and then concentrated under reduced pressure to give a residue which was purified by inverse chromatography on fast silica column (Column: 80 g Agela C18, Mobile Phase A: water, Mobile Phase B: acetonitrile, Flow rate: 80 mL/min, gradient condition: from 5% B to 40% B) to yield intermediate 13 (1.40 g) as a white solid.

The intermediates reported below were prepared following an analogous methodology as described for intermediate 13 starting from the corresponding intermediates:

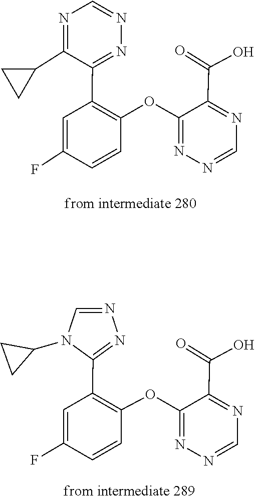 [SMILES: O=C(O)c1ncnnc1Oc1ccc(F)cc1-c1nncnc1C1CC1 ; O=C(O)c1ncnnc1Oc1ccc(F)cc1-c1nncn1C1CC1]

### Preparation of Intermediate 298

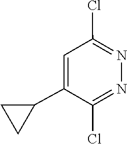 [SMILES: Clc1cc(C2CC2)c(Cl)nn1]

To the mixture of 3,6-dichloropyridazine (20.0 g, 134 mmol) in DCM (660 mL) and H₂O (600 mL) were added cyclopropanecarboxylic acid (23.0 g, 267 mmol), 1-(chloromethyl)-4-fluoro-1,4-diazabicyclo[2.2.2]octane-1,4-diiumtetrafluoroborate (95.0 g, 268 mmol) and TFA (10.0 mL, 135 mmol) at 25° C. under N₂ atmosphere. The resulting mixture was stirred at 25° C. for 5 min, then AgNO₃ (68.0 mL, 27.2 mmol, 0.4 M in H₂O) was added, the resulting mixture was stirred at 55° C. for 10 h under N₂ atmosphere. After cooling to RT, the reaction mixture was quenched with 2 N NaOH (90 mL) and extraction with EtOAc (500 mL×3), the combined organic layers were dried over Na₂SO₄. After filtration and concentration, the crude residue was purified by preparative HPLC (YMC-Triart Prep C18 250*50 mm*10 um, mobile phase A: water (0.225% formic acid), mobile phase B: ACN, flow rate: 100 mL/min, gradient condition from 15% B to 55% B). The desired fractions were collected and lyophilized to afford intermediate 298 (6.00 g, 24% yield) as a colorless oil.

### Preparation of Intermediate 299 and Intermediate 300

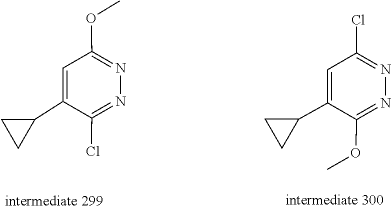

To a solution of intermediate 298 (6.00 g, 28.6 mmol) in MeOH (50 mL) was added sodium methanolate (7.72 g, 143 mmol) at 25° C. under N₂ atmosphere in portions and the reaction was stirred at this temperature for 0.5 h. The resulting mixture was quenched with 1N HCl (100 mL) to adjust the pH value to 7 and extracted with EtOAc (135 mL×3). The combined organic layers were dried over anhydrous Na₂SO₄. After filtration, the filtrate was concentrated in vacuo to afford a mixture consisting of intermediate 299 and intermediate 300 (5.6 g, crude) as a colorless oil which was used directly in next step without further purification.

### Preparation of Intermediate 301

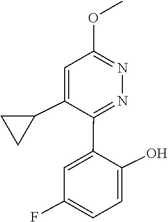 [SMILES: COc1cc(C2CC2)c(-c2cc(F)ccc2O)nn1]

To the solution of a mixture consisting of intermediate 299 and intermediate 300 (5.60 g, crude) in dioxane (120 mL) and H₂O (24 mL) was added (5-fluoro-2-hydroxyphenyl)boronic acid (9.63 g, 61.7 mmol), Na₂CO₃ (9.82 g, 92.6 mmol) and Pd(PPh₃)₄ (1.78 g, 1.54 mmol). The resulting mixture reaction was stirred at 90° C. for 8 h under N₂ atmosphere. After cooling to RT, the reaction mixture was concentrated under reduced pressure and the crude residue was purified by FCC (from PE to PE/EtOAc=3/1) to afford intermediate 301 (1.20 g) as a white solid.

### Preparation of Intermediate 302

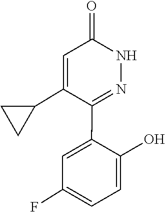 [SMILES: O=c1cc(C2CC2)c(-c2cc(F)ccc2O)n[nH]1]

To a solution of intermediate 301 (1.80 g, 6.92 mmol) in ACN (40 mL) were added cerium(III) chloride (2.56 g, 10.4 mmol) and NaI (1.56 g, 10.4 mmol). The resulting mixture was stirred at 70° C. for 8 h. After cooling to RT, the mixture was filtered and the filter cake was washed with EtOAc (30 mL×2). The filtrate was concentrated under reduced pressure and the crude residue was purified by FCC (from PE to pure EtOAc) to afford intermediate 302 (1.4 g, 74% yield) as a white solid.

### Preparation of Intermediate 14

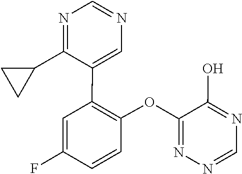 [SMILES: Oc1ncnnc1Oc1ccc(F)cc1-c1cncnc1C1CC1]

1,3-dibromo-1,3,5-triazinane-2,4,6-trione (1.22 g, 4.25 mmol) was added to a solution of intermediate 13 (1.00 g, 2.83 mmol) in DCE (20 mL). The resultant mixture was stirred at room temperature for 0.5 hours. The mixture was quenched with H₂O (1 mL), filtered, the filter cake was washed with CH₂Cl₂ (10 mL×2), the filtrate was concentrated under reduced pressure to give the crude product, which was purified by FCC (eluting with ethyl acetate:methanol=100:0 to 10:1) to yield intermediate 14 (600 mg, yield 60.8%) as a yellow solid.

### Alternative Preparation of Intermediate 14

To a solution of intermediate 13 (700 mg, 1.98 mmol) in DCE (30 mL) was added 1,3-dibromo-1,3,5-triazinane-2,4,6-trione (900 mg, 3.14 mmol). The resultant mixture was stirred at room temperature for 0.5 hours. The suspension was isolated via filtration. The filter cake was purified by FCC (EtOAc:MeOH=10:1) to yield intermediate 14 (500 mg, 73% yield) as a light brown solid.

### Preparation of Intermediate 15

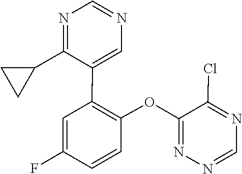 [SMILES: Fc1ccc(Oc2nncnc2Cl)c(-c2cncnc2C2CC2)c1]

To a solution of intermediate 14 (200 mg, 0.615 mmol) in CH₂Cl₂ (10 mL) was added oxalyl chloride (134 μL, 1.23 mmol) followed by 2 drops of DMF at room temperature. The mixture was stirred at this temperature for 1.5 h. The mixture was concentrated under reduced pressure to yield intermediate 15 (200 mg, crude) as a brown solid, which was used as such directly for the next reaction step.

### Example A4

### Preparation of Intermediate 17

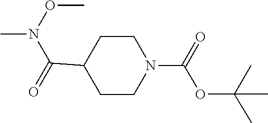 [SMILES: CON(C)C(=O)C1CCN(C(=O)OC(C)(C)C)CC1]

HATU (99.5 g, 262 mmol) was added in portions to a 0° C. (ice/water) mixture of 1-(tert-butoxycarbonyl)piperidine-4-carboxylic acid (50.0 g, 218 mmol), N,O-dimethylhydroxylamine hydrochloride (23.4 g, 240 mmol), and Et₃N (90.9 mL, 654 mmol) in dichloromethane (500 mL). The reaction mixture was stirred at room-temperature for 12 hours and then concentrated to dryness under reduced pressure. The residue was diluted with water (1500 mL) and extracted with dichloromethane (500 mL×3). The combined organic extracts were dried over anhydrous Na₂SO₄, filtered, and concentrated to dryness under reduced pressure to afford the crude product, which was purified by FCC (eluent: petroleum ether:ethyl acetate=1:0 to 1:1) to yield intermediate 17 (54 g) as a yellow oil.

The intermediates reported below were prepared following an analogous methodology as described for intermediate 17 starting from the corresponding intermediates or starting materials:

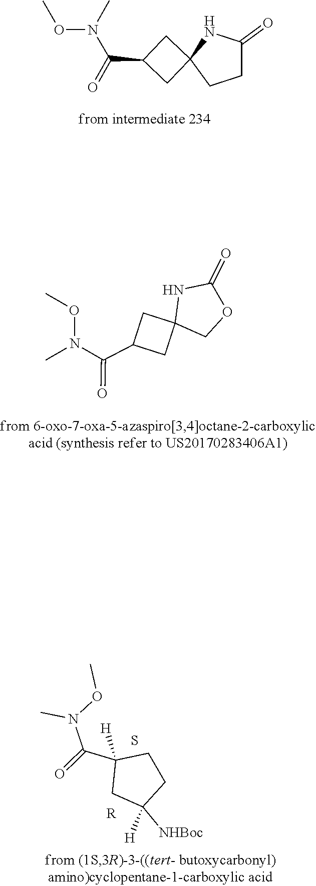

### Preparation of Intermediate 18

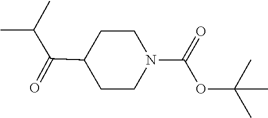 [SMILES: CC(C)C(=O)C1CCN(C(=O)OC(C)(C)C)CC1]

Intermediate 17 (54.0 g, 198 mmol) and THE (500 mL) were added into a 1 L three-necked round-bottomed flask. Then, i-PrMgCl (198 mL, 397 mmol, 2 M in THF) was added dropwise into the mixture at 0° C. (ice/water) under N₂. The mixture was stirred with warming to room temperature for 10 hours before pouring into water (2000 mL), and extracted with EtOAc (1000 mL×3). The organic layer was washed with brine, dried over Na₂SO₄, filtered and concentrated under reduced pressure to give the crude which was purified by FCC on silica gel (eluent: petroleum ether:ethyl acetate from 1:0 to 2:1, TLC: petroleum ether:ethyl acetate=2:1, R_{f}=0.6) to yield intermediate 18 (19.2 g) as a yellow oil.

The intermediates reported below were prepared following an analogous methodology as described for intermediate 18 starting from the corresponding intermediates:

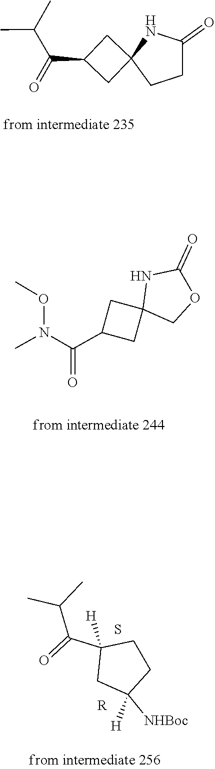

### Example A5

### Preparation of Intermediate 22

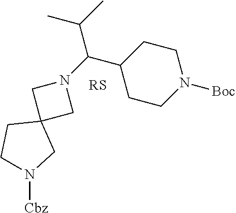 [SMILES: CC(C)C(C1CCN(C(=O)OC(C)(C)C)CC1)N1CC2(CCN(C(=O)OCc3ccccc3)C2)C1]

To a solution of intermediate 3 (6.00 g, 24.4 mmol), intermediate 18 (6.22 g, 24.4 mmol) in dry methanol (180 mL) was added ZnCl₂ (6.64 g, 48.7 mmol). The reaction mixture was heated and stirred at 65° C. for 3 h and then, NaBH₃CN (4.59 g, 73.1 mmol) was added. The reaction mixture was stirred at 65° C. for 12 hours. Then an additional amount of intermediate 18 was added (6.22 g, 24.4 mmol) and the reaction mixture was stirred at 65° C. for another 20 hours. The reaction mixture was cooled to room temperature, suspended into sat. NaHCO₃ (180 mL) and stirred for 30 min. The mixture was filtered and the filter cake was washed with EtOAc (50 mL). The filtrate was extracted with EtOAc (200 mL×3). The combined organic layers were dried over anhydrous Na₂SO₄, filtered and concentrated under reduced pressure to give a residue, which was purified by FCC on silica gel (eluent: petroleum ether:ethyl acetate from 1:0 to 0:1, TLC: petroleum ether:ethyl acetate=0:1, R_{f}=0.3) to yield intermediate 22 (9.80 g) as a colorless oil.

### Preparation of Intermediates 23 and 24

### Intermediate 23

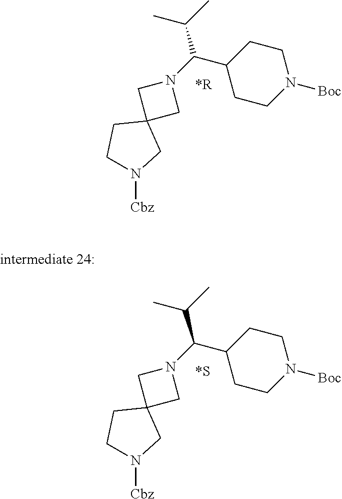

Intermediate 22 (50.0 g, 103 mmol) was further purified by SFC over DAICEL CHIRALPAK AD (isocratic elution: i-PrOH (containing 0.1% of 25% aq. NH₃): supercritical CO₂, 25%: 75% to 25%: 75% (v/v)). The pure fractions were collected and the volatiles were removed under reduced pressure to yield intermediate 23 (22 g, 44% yield) as a yellow oil and intermediate 24 (23 g, 46% yield) as a yellow oil.

### Preparation of Intermediate 26

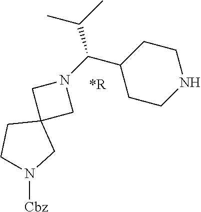 [SMILES: CC(C)[C@H](C1CCNCC1)N1CC2(CCN(C(=O)OCc3ccccc3)C2)C1]

HCl/1,4-dioxane (10 mL, 40 mmol) was added to a solution of intermediate 23 (1.0 g, 2.1 mmol) in 1,4-dioxane (10 mL). The reaction mixture was stirred at room-temperature for 3 hours. The reaction mixture was concentrated to dryness under reduced pressure, then NH₃H₂O (5 mL; concentrated, typically 25-28%)) was added into the mixture. The residue was suspended in water (10 mL) and the mixture was frozen using dry ice/acetone and then lyophilized to dryness to yield intermediate 26 (900 mg, crude), as a yellow solid, which was used in the next step without further purification.

### Preparation of Intermediate 27

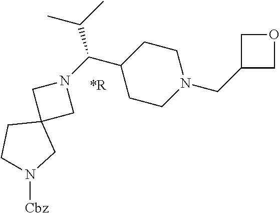 [SMILES: CC(C)[C@H](C1CCN(CC2COC2)CC1)N1CC2(CCN(C(=O)OCc3ccccc3)C2)C1]

TEA (1.3 mL, 9.3 mmol) was added to a solution of intermediate 26 (900 mg, crude) in dichloromethane (10 mL). Oxetane-3-carbaldehyde (310 mg, 14 mmol) was added to the above solution, and the resultant mixture was stirred at room-temperature of 0.5 hours. Then NaBH(OAc)₃ (1.5 g, 7.1 mmol) was added to the above solution, and the resultant mixture was stirred at room-temperature of 1.5 hours. The reaction mixture was diluted with dichloromethane (30 mL) and washed with sat. NaHCO₃ (10 mL×3). The organic layer was dried over anhydrous Na₂SO₄, filtered and concentrated under reduced pressure to give the crude product, which was purified by preparative HPLC using a Waters Xbridge Prep OBD C18 150*40 mm*10 um with water (0.05% ammonia hydroxide v/v)/ACN from 100/0 to 20/80 (v/v) to afford pure product. The product was suspended in water (10 mL), the mixture frozen using dry ice/acetone, and then lyophilized to dryness to yield intermediate 27 (500 mg), as a colorless oil.

### Preparation of Intermediate 28

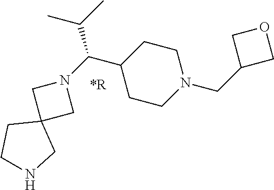 [SMILES: CC(C)[C@H](C1CCN(CC2COC2)CC1)N1CC2(CCNC2)C1]

Intermediate 27 (500 mg, 1.10 mmol), and dry Pd/C (150 mg, w/w %=10% Pd loading) were suspended in THE (30 mL). The reaction mixture was stirred at 45° C. for 4 hours under H₂ (50 Psi). The suspension was filtered through a pad of Celite® which was washed with THE (50 mL). The filtrate was concentrated to dryness under reduced pressure to yield intermediate 28 (350 mg) as a colorless oil which was used in the next step without further purification.

### Example A6

### Preparation of Intermediate 25

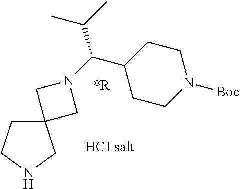 [SMILES: CC(C)[C@H](C1CCN(C(=O)OC(C)(C)C)CC1)N1CC2(CCNC2)C1]

Dry Pd/C (1 g) was added to a mixture of intermediate 23 (7.5 g, 15 mmol), 1,1,2-trichloroethane (2.3 mL, 25 mmol) and MeOH (200 mL) under Ar. The mixture was stirred under H₂ (50 psi) at 40° C. for 4 hours. The mixture was filtered and the filtrate was concentrated to dryness to yield intermediate 25 as a white solid (5.8 g, HCl salt, 97% yield).

The intermediate reported below was prepared following an analogous methodology as described for intermediate 25 starting from the corresponding intermediate:

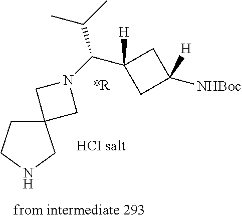 [SMILES: CC(C)[C@H]([C@H]1C[C@@H](NC(=O)OC(C)(C)C)C1)N1CC2(CCNC2)C1]

### Example A7

### Preparation of Intermediate 1

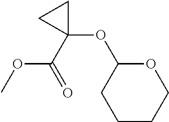

Pyridinium-p-toluene-sulfonic acid (2.16 g, 8.61 mmol) was added to a solution of methyl 1-hydroxycyclopropanecarboxylate (10.0 g, 86.1 mmol) and 3-4-dihydropyran (7.68 g, 91.3 mmol) in DCM (100 mL). After addition, the reaction mixture was stirred at 20° C. overnight. The reaction was washed with H₂O (70 mL), saturated aqueous brine solution (50 mL), dried over sodium sulfate and concentrated in vacuum to afford an oil. The oil was purified by FCC (PE:EA=10:1) to yield intermediate 1 (13.5 g, 78% yield) as a colorless oil.

### Preparation of Intermediate 4

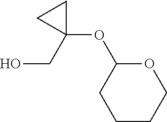 [SMILES: OCC1(OC2CCCCO2)CC1]

To a solution of LiAlH₄ (2.00 g, 52.7 mmol) in 80 mL of THF was added intermediate 1 (8.00 g, 40.0 mmol) in 20 mL of THF at 0° C. under N₂ atmosphere. After addition, the reaction mixture was stirred at 0° C. for 2 hours. The reaction mixture was cooled to 0° C., and then water (2 mL), 10% NaOH aq. (2 ml), water (6 mL) and 20 g Na₂SO₄ were added sequentially to the reaction mixture. The resulting mixture was filtered. The filter cake was washed with THF (80 mL), and the combined filtrate was concentrated under reduced pressure, to obtain the title Intermediate 3A (6.23 g, 81% yield) as a colorless oil.

### Preparation of Intermediate 9

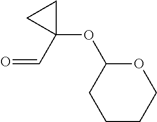 [SMILES: O=CC1(OC2CCCCO2)CC1]

To a solution of intermediate 4 (4.00 g, 23.2 mmol) in 100 mL of DCM was added Dess-Martin periodinane (16.0 g, 37.7 mmol). After addition, the reaction mixture was stirred at 15° C. for 1.5 hours. The reaction mixture was diluted with 50 mL of DCM and stirred with 60 mL of sat. NaHCO₃ and 60 mL of sat. Na₂S₂O₃ for 10 minutes. The mixture was then extracted with DCM (50 mL) three times. Then brine (100 mL) was added, the organic and brine layers were separated, and the combined organic layers were dried over Na₂SO₄, filtered and concentrated in vacuum to yield intermediate 9 (2.98 g, 70% yield) as a light yellow oil.

### Example A8

### Preparation of Intermediate 29

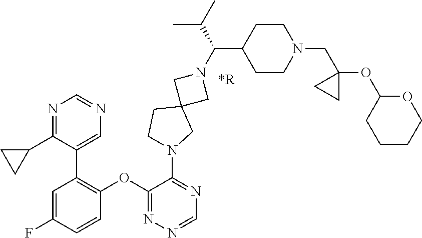 [SMILES: CC(C)[C@H](C1CCN(CC2(OC3CCCCO3)CC2)CC1)N1CC2(CCN(c3ncnnc3Oc3ccc(F)cc3-c3cncnc3C3CC3)C2)C1]

To a solution of compound 3 (240 mg, crude) and intermediate 9 (500 mg, 2.94 mmol) in DCM (20 mL) was added TEA (363 mg, 3.59 mmol). The mixture was stirred at room temperature for 10 minutes, then NaBH₃CN (300 mg, 4.77 mmol) was added in portions. The reaction mixture was stirred at rt overnight. The mixture was diluted with DCM (50 mL), washed with H₂O (20 mL) and brine (20 mL). The organic layer was dried over anhydrous Na₂SO₄, filtered and concentrated under reduced pressure to yield intermediate 29 (250 mg, crude) as a light brown oil (used as such for the next reaction step without further purification).

### Example A9

### Preparation of Intermediate 30

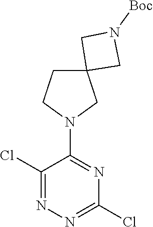 [SMILES: CC(C)(C)OC(=O)N1CC2(CCN(c3nc(Cl)nnc3Cl)C2)C1]

At room temperature, tert-butyl 2,6-diazaspiro[3.4]octane-2-carboxylate (1.27 g; 5.96 mmol) and triethylamine (1.7 mL; 11.93 mmol) were added to a stirred solution of trichlorotriazine (1.1 g; 5.96 mmol) in DCM (40 mL). The reaction mixture was stirred overnight at room temperature and, then, diluted with water and extracted with DCM. The organic layer was washed with water and brine, dried (MgSO₄), filtered, and concentrated. The residue was taken with Et₂O. The precipitate was filtered and dried to give 1.76 g of intermediate 30 (81%).

### Preparation of Intermediate 31

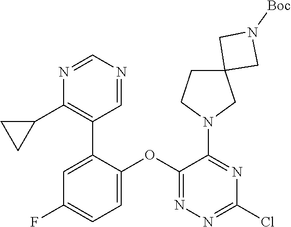 [SMILES: CC(C)(C)OC(=O)N1CC2(CCN(c3nc(Cl)nnc3Oc3ccc(F)cc3-c3cncnc3C3CC3)C2)C1]

A mixture of intermediate 30 (3.25 g; 9.017 mmol), intermediate 11 (2.18 g; 9.468 mmol) and cesium carbonate (3.23 g; 9.919 mmol) in DMF (100 mL) was stirred at r.t. overnight. The solution was cooled to r.t., poured into cold water and extracted with EtOAc. The organic layer was decanted, washed with water, then brine, dried over MgSO₄, filtered and evaporated to dryness. The residue (5.8 g) was purified by chromatography over silica gel (irregular SiOH, 40 g+80 g; mobile phase: gradient from 40% EtOAc, 60% heptane to 100% EtOAc, 0% heptane). The pure fractions were collected and evaporated to dryness yielding 3.41 g of (68%) intermediate 31 and 600 mg of an impure fraction which was gathered with another impure fraction (700 mg) coming from a reaction performed on 1 g of intermediate 30. The resulting residue was purified by chromatography over silica gel (irregular SiOH, 24 g+24 g; mobile phase: gradient from 40% EtOAc, 60% heptane to 100% EtOAc, 0% heptane). The pure fractions were collected and evaporated to dryness yielding additional 1.04 g of intermediate 31.

### Preparation of Intermediate 32

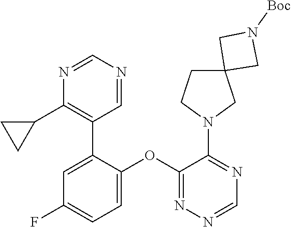 [SMILES: CC(C)(C)OC(=O)N1CC2(CCN(c3ncnnc3Oc3ccc(F)cc3-c3cncnc3C3CC3)C2)C1]

A mixture of intermediate 31 (500 mg; 0.902 mmol), Pd/C (144 mg; 0.135 mmol) in MeOH (25 mL) and triethylamine (125 μL; 0.902 mmol) was hydrogenated under a pressure of H₂ (1 bar) for 40 min. The catalyst was removed by filtration through a pad of Celite® and washed with DCM. The filtrate was washed with water, decanted, filtered through Chromabond® and evaporated to dryness. The residue (520 mg) was purified by chromatography over silica gel (irregular SiOH, 24 g; mobile phase: gradient from 0% NH₄OH, 0% MeOH, 100% DCM to 0.5% NH₄OH, 5% MeOH, 95% DCM). The pure fractions were collected and evaporated to dryness yielding 300 mg (64%) of intermediate 32.

### Alternative Preparation

A mixture of intermediate 31 (13.60 g, 24.58 mmol), 1,1′-Bis(diphenylphosphino)ferrocene-palladium(II)dichloride dichloromethane complex (1.00 g, 1.23 mmol), sodium borohydride (1.58 g, 41.73 mmol) and N,N,N′,N′-tetramethylethylenediamine (6.3 mL, 41.73 mmol) in THE (280 mL) was stirred overnight at room temperature under an atmosphere of nitrogen. The reaction mixture was quenched with water (250 mL) and extracted with EtOAc (4×250 mL). The combined organic layers were washed with water (600 mL), brine (600 mL), dried over anhydrous Na₂SO₄, filtered and concentrated under reduced pressure. The crude product was purified by flash column chromatography over silica gel (mobile phase: EtOAc/petroleum ether 10:1). The pure fractions were collected and evaporated to dryness yielding 10.4 g (79%) of intermediate 32 as light a yellow oil.

### Preparation of Intermediate 33

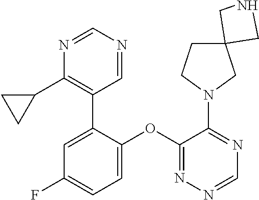 [SMILES: Fc1ccc(Oc2nncnc2N2CCC3(CNC3)C2)c(-c2cncnc2C2CC2)c1]

TFA (1 mL; 13.067 mmol) was added to a solution of intermediate 32 (300 mg; 0.577 mmol) in DCM (10 mL) and the reaction mixture was stirred for 4 hours at room temperature. The reaction was diluted with ACN and evaporated to dryness several times. The residue was then dissolved in DCM and basified with diluted 15% aqueous NH₄OH. The organic layer was decanted, washed again with water, filtered through Chromabond® and evaporated to dryness yielding 245 mg of intermediate 33.

### Preparation of Intermediate 34

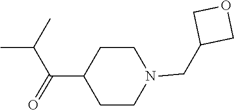 [SMILES: CC(C)C(=O)C1CCN(CC2COC2)CC1]

Under N₂, to a solution of 2-methyl-1-(4-piperidinyl)-1-propanone (450 mg; 0.23 mmol), oxetane-3-carbaldehyde (1 g; 5.22 mmol) in DCM (50 mL) was added triethylamine (4.65 mL; 26.13 mmol). The reaction mixture was stirred at rt for 15 min, then NaBH(OAc)₃ (3.32 g; 15.7 mmol) was added by portion and the reaction was stirred at r.t. overnight. The reaction mixture was diluted with water, extracted with DCM (×2) and washed with brine. The organic layer was dried over MgSO₄, filtered and evaporated to dryness. The residue (1.4 g) was purified by silica gel chromatography (Stationary phase: irregular SiOH 15-40 μm 40 g, Mobile phase: Gradient from 100% HEPTANE, 0% EtOAc to 80% HEPTANE, 20% EtOAc) yielding 0.88 g of (75%) of intermediate 34.

### Example A10

### Preparation of Intermediate 35

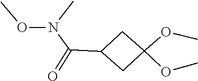

To a solution of 3,3-dimethoxycyclobutanecarboxylic acid (12.0 g, 75 mmol) in DCM (145 mL) was added T₃P (100 mL, 168 mmol. 50% in EtOAc) and DIEA (64 mL, 372 mmol) at 0° C. Then N,O-dimethylhydroxylamine hydrochloride (8.8 g, 89.5 mmol) was added at 0° C. The mixture was stirred at room temperature for 16 hours. The mixture was poured onto a saturated solution NaHCO₃ and EtOAc was added. The organic layer was separated, washed with brine, dried over MgSO₄, filtered and concentrated under reduced pressure to give intermediate 35 (16.0 g, crude) which was used in the next step without further purification.

### Preparation of Intermediate 36

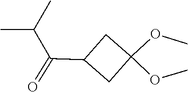

The reaction was performed twice on 15.7 g of intermediate 35 and respective reaction media were mixed for the work-up and purification.

To a solution of intermediate 35 (15.7 g, 77.7 mmol) in THE (420 mL) was added isopropylmagnesium chloride (178.5 mL, 232 mmol, 2 M in THF) dropwise at 0° C. under N₂ atmosphere. The reaction mixtures were stirred at room temperature for 12 hours under N₂ atmosphere and then, poured onto ice-water and a 10% aqueous solution of NH₄Cl. The mixture obtained was combined with the mixture obtained from the 2^{nd} reaction, and the combined mixture was extracted with EtOAc. The combined organic layers were washed with brine, dried over MgSO₄, filtered and concentrated under reduced pressure. The crude product was purified by flash column chromatography over silica gel (mobile phase: Heptane:EtOAc 9:1). The pure fractions were collected and evaporated to dryness yielding 22 g (76%) of intermediate 36 as a colourless oil.

### Example A11

### Preparation of Intermediate 37, 37a (*S) and 37b (*R

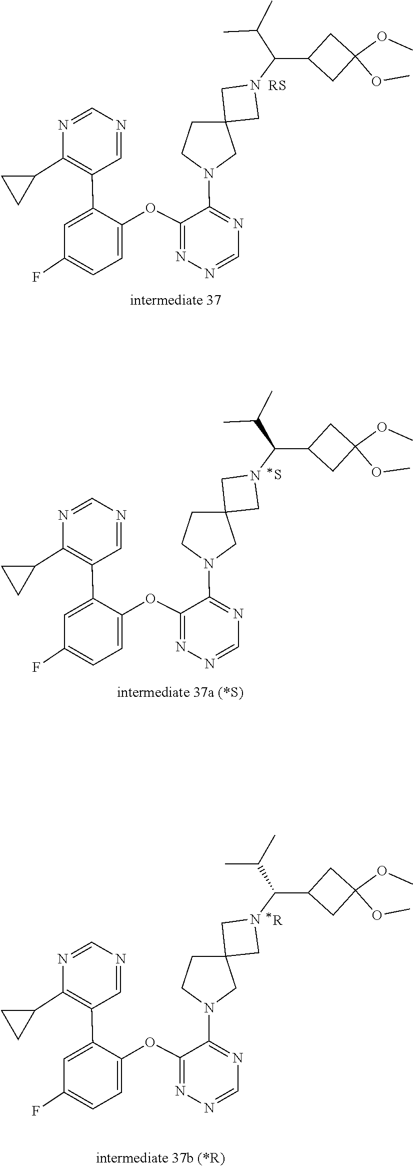

The reaction was performed twice: once on 5.09 g of intermediate 33, and once on 10.9 g of intermediate 33. The resulting crude mixtures were combined for the work up and purification.

A mixture of intermediate 33 (5.09 g; 12.14 mmol), intermediate 36 (2.26 g; 12.14 mmol), AcOH (764 μL; 13.35 mmol) and NaBH₃CN (763 mg; 12.14 mmol) in MeOH (50 mL) was stirred at 50° C. overnight. The two reaction mixtures were combined and poured onto a 10% aqueous solution of K₂CO₃. DCM was added. The layers were separated and then, the aqueous layer was extracted with DCM (3×). The organic layer was dried over MgSO₄, filtered and the solvent was evaporated. The crude product was purified by chromatography over silica gel (mobile phase: from 100% DCM to 95% DCM, 5% MeOH, 0.5% NH₄OH). The pure fractions were collected and the solvent was evaporated to afford 7.84 g of intermediate 37. This residue was combined with other batches coming from the same reaction performed on 10.9 g of intermediate 33. Then, resulting intermediate 37 (18 g) was purified by chiral SFC (CHIRALPAK AD-H 5 μm 250*30 mm, mobile phase: 78% CO₂, 22% EtOH (0.3% iPrNH₂)). The pure fractions were collected and the solvent was evaporated to give 9.04 g of intermediate 37a (*S) (ee100%) and 8.88 g of intermediate 37b (*R) as an off-white foam (ee 98.9%).

### Preparation of Intermediate 38, 38a and 38b

### Intermediate 38

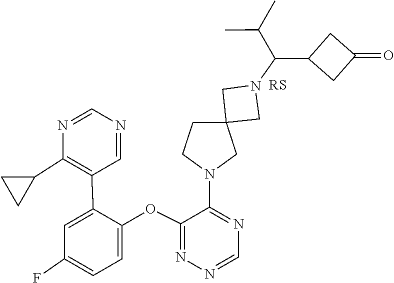 [SMILES: CC(C)C(C1CC(=O)C1)N1CC2(CCN(c3ncnnc3Oc3ccc(F)cc3-c3cncnc3C3CC3)C2)C1]

At 5° C., TFA (4 mL; 52.7 mmol) was added dropwise to a solution of intermediate 37 (1.55 g; 2.63 mmol) in DCM (40 mL) and the reaction mixture was stirred overnight at rt. The mixture was diluted with ACN and evaporated to dryness. The residue was dissolved in DCM and basified with a 30% aqueous solution of NH₄OH at 0-5° C. The mixture was stirred at rt for 1 h. The organic layer was decanted, washed with water, dried over MgSO₄, filtered and the solvent was evaporated to give 1.4 g (100%) of intermediate 38 as an off-white foam.

### Intermediate 38b

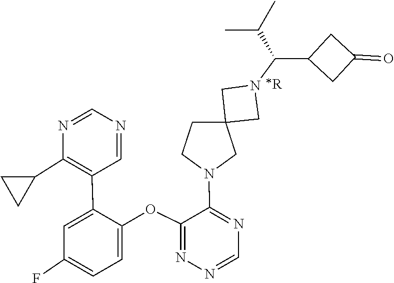

The reaction was performed twice on 4.44 g of intermediate 37b (*R) and the obtained mixtures were combined for the work up.

At 5° C., TFA (11.5 mL; 150.6 mmol) was added dropwise to a solution of intermediate 37b (*R) (4.44 g; 7.53 mmol) in DCM (110 mL) and the reaction mixture was stirred for 18 h at rt. The mixture obtained was combined with the mixture obtained from the 2^{nd} reaction, and the combined mixture was diluted with ACN and evaporated to dryness. The residue was dissolved in DCM and basified with a 30% aqueous solution of NH₄OH at 0-5° C. The mixture was stirred at rt for 1 h. The organic layer was decanted, washed with water, dried over MgSO₄, filtered and the solvent was evaporated to give 7.87 g (96%) of intermediate 38b (*R) as an off-white foam.

### Intermediate 38a

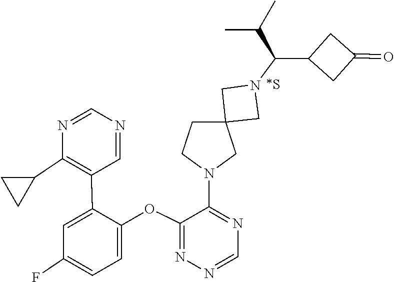

At 5° C., TFA (13 mL; 170 mmol) was added dropwise to a solution of intermediate 37a (*S) (5 g; 8;48 mmol) in DCM (130 mL) and the reaction mixture was stirred for 4 h at rt. The mixture was diluted with ACN and evaporated to dryness. The residue was dissolved in DCM and basified with a 30% aqueous solution of NH₄OH at 0-5° C. The mixture was stirred at rt for 1 h. The organic layer was decanted, washed with water, dried over MgSO₄, filtered and the solvent was evaporated to give 4.6 g (100%) of intermediate 38a (*S) as an off-white foam.

### Example A12

### Preparation of Intermediate 39

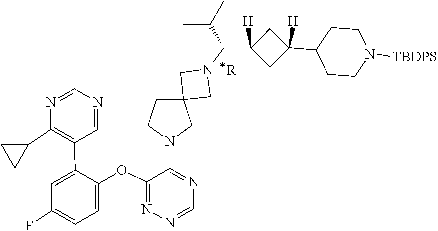

NaBH₃CN (278 mg; 4.42 mmol) was added to a mixture of intermediate 38b (1.2 g; 2.21 mmol), tert-butyl-diphenyl-(4-piperidyloxy)silane (2.4 g; 7.1 mmol), AcOH (126 μL; 2.21 mmol) in MeOH (65 mL). Then, the reaction mixture was heated at 60° C. for 48 h. The reaction mixture was cooled to r.t, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.7% NH₄OH, 7% MeOH, 93% DCM). The pure fractions were collected and evaporated to dryness to give 1.09 g (57%) of intermediate 39 (*R).

### Example A13

### Preparation of Intermediate 40

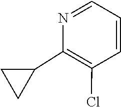

To a solution of 2,3-dichloropyridine (6.0 g, 40.54 mmol) in THE (210 mL) in N-Methyl-2-pyrrolidinone (54 mL) was added ferric acetylacetonate (530 mg, 1.50 mmol). Then, cyclopropylmagnesium bromide (47 mL, 46.63 mmol) was added at 0° C. After stirring for 1 h at room temperature, additional cyclopropylmagnesium bromide (23 mL, 23.313 mmol) was added. After stirring for 2 h at room temperature, the reaction mixture was quenched with a saturated aqueous solution of NH₄Cl. The solid was filtered out and the filtrate was extracted with EtOAc. The organic layers were combined, dried with Na₂SO₄, filtered and concentrated under reduced pressure. The residue was purified by flash chromatography column (mobile phase: ethyl acetate/hexane, 1:20) to give 3.0 g (48% yield) of the intermediate 40 as a light yellow oil.

### Preparation of Intermediate 41

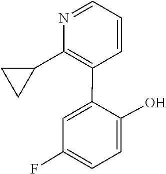 [SMILES: Oc1ccc(F)cc1-c1cccnc1C1CC1]

To a solution of intermediate 40 (4.5 g, 29.30 mmol) in 1,4-dioxane (90 mL) were added 4-fluoro-2-hydroxyphenylboronic acid (4.6 g, 29.30 mmol), Pd(amphos)Cl₂ (1.0 g, 1.46 mmol) and Na₂CO₃ (30 mL, 2 M in water). After stirring for 2 h at 90° C., the reaction mixture was cooled to room temperature, quenched with water and extracted with EtOAc. The combined organic layers were dried with Na₂SO₄, filtered and concentrated under reduced pressure. The residue was purified by flash chromatography column (mobile phase: ethyl acetate/hexane, 1:2) to give 5.5 g (81.1% yield) of the intermediate 41 as a yellow solid.

### Preparation of Intermediate 42

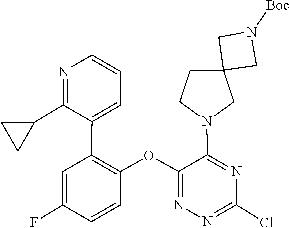 [SMILES: CC(C)(C)OC(=O)N1CC2(CCN(c3nc(Cl)nnc3Oc3ccc(F)cc3-c3cccnc3C3CC3)C2)C1]

To a solution of intermediate 41 (5.5 g, 24.0 mmol) in THE (137 mL) were added intermediate 30 (8.6 g, 24.0 mmol) and DBU (3.6 g, 24.0 mmol). After stirring overnight at room temperature, the reaction mixture was quenched with water and extracted with EtOAc. The combined organic layers were dried with Na₂SO₄, filtered and concentrated under reduced pressure. The residue was purified by flash chromatography column (mobile phase: ethyl acetate/hexane, 1:1) to give 8.8 g (63.5% yield) of the intermediate 42 as a yellow solid.

### Preparation of Intermediate 43

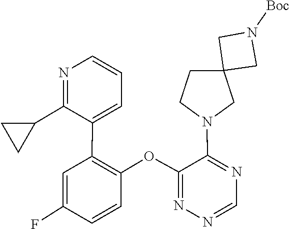 [SMILES: CC(C)(C)OC(=O)N1CC2(CCN(c3ncnnc3Oc3ccc(F)cc3-c3cccnc3C3CC3)C2)C1]

Intermediate 42 (6.7 g, 12.12 mmol) in THE (167 mL) were added 1,1′-bis(diphenylphosphino)ferrocene-palladium(II)dichloride dichloromethane complex (989 mg, 1.21 mmol), sodium borohydride (779 mg, 20.60 mmol) and N,N,N′,N′-tetramethylethylenediamine (2.4 g, 20.60 mmol). After stirring overnight at room temperature, the reaction mixture was quenched with water and extracted with EtOAc. The combined organic layers were dried with Na₂SO₄, filtered and concentrated under reduced pressure. The residue was purified by flash chromatography column (mobile phase: ethyl acetate/hexane, 2:3) to give 6.1 g (95.5% yield) of the intermediate 43 as a yellow brown solid.

### Preparation of Intermediate 44

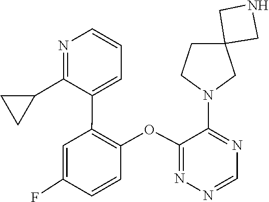 [SMILES: Fc1ccc(Oc2nncnc2N2CCC3(CNC3)C2)c(-c2cccnc2C2CC2)c1]

At 0° C., TFA (16 mL; 212 mmol) was added to a solution of intermediate 43 (7.33 g; 14.1 mmol) in DCM (150 mL) and the reaction mixture was stirred for 5 hours at room temperature. The reaction was concentrated under vacuum. The residue was dissolved in 40 mL of water and the solution was basified with 15% aqueous solution of NH₄OH. The aqueous layer was extracted with DCM (*3). The organic layer was decanted, washed again with brine, dried over MgSO₄, filtered and evaporated to dryness to give 6.3 g of intermediate 44 which was used in the next step without further purification.

### Preparation of Intermediate 45, 45a (*R) and 45b (*S

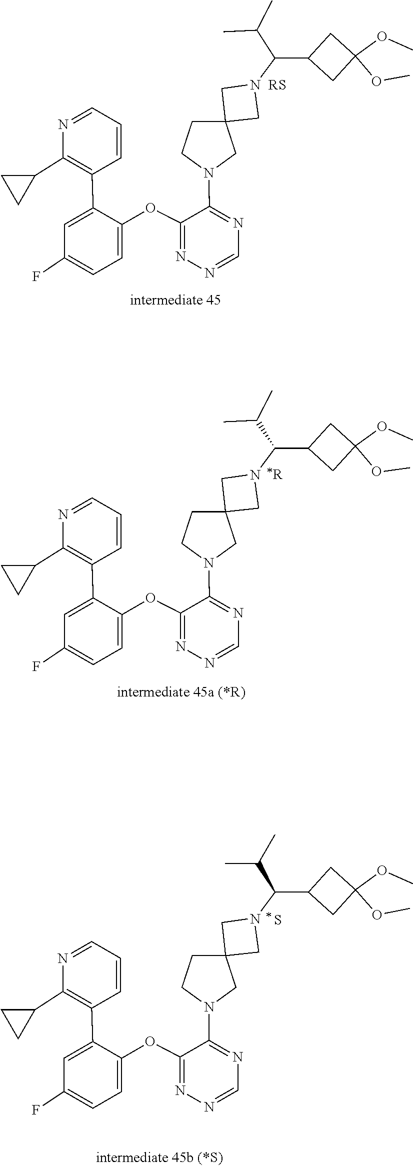

A mixture of intermediate 44 (5 g; 11.2 mmol), intermediate 36 (2.51 g; 13.5 mmol), AcOH (707 μL; 12.4 mmol) and NaBH₃CN (2.1 g; 34 mmol) in MeOH (47 mL) was stirred at 50° C. overnight. The reaction mixture was poured onto a 10% aqueous solution of K₂CO₃ and DCM was added. The mixture was extracted with DCM (3×). The organic layer was dried over MgSO₄, filtered and the solvent was evaporated. The crude product was purified by chromatography over silica gel (mobile phase: from 99% DCM, 1% i-PrOH to 88% DCM, 12% i-PrOH). The pure fractions were collected and the solvent was evaporated. This residue (4.6 g) was purified by chiral SFC (CHIRALPAK AD-H 5 μm 250*30 mm, mobile phase: 85% CO₂, 15% EtOH (0.3% iPrNH₂)). The pure fractions were collected and the solvent was evaporated to give 1.98 g (30%) of intermediate 45a (*R) (ee 100%) and 2.09 g (31%) of intermediate 45b (*S) as an off-white foam (ee 99.4%).

### Preparation of Intermediate 46a (*R) and 46b (*S

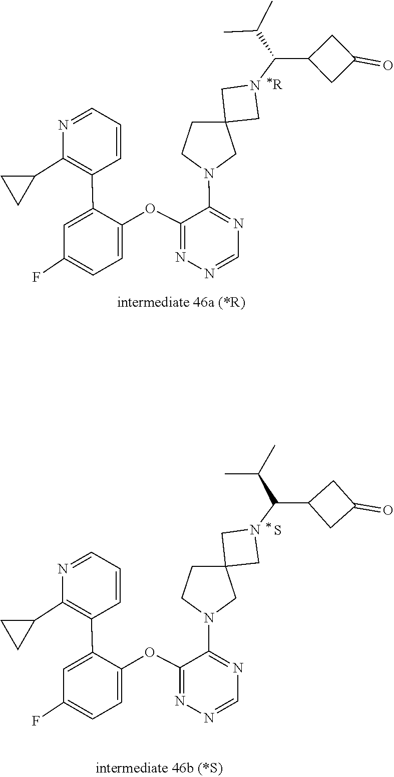

At 5° C., TFA (5.1 mL; 67 mmol) was added dropwise to a solution of intermediate 45a (*R) (1.98 g; 3.36 mmol) in DCM (76 mL) and the reaction mixture was stirred for 2 h at rt. The reaction was evaporated to dryness. The residue was dissolved in DCM and basified with a 30% aqueous solution of NH₄OH at 0-5° C. The mixture was stirred at rt for 1 h. The organic layer was decanted, washed with water, dried over MgSO₄, filtered and the solvent was evaporated to give 1.90 g (100%) of intermediate 46a (*R).

At 5° C., TFA (5.4 mL; 71 mmol) was added dropwise to a solution of intermediate 45b (*S) (2.09 g; 3.55 mmol) in DCM (81 mL) and the reaction mixture was stirred for 2 h at rt. The reaction was evaporated to dryness. The residue was dissolved in DCM and basified with a 30% aqueous solution of NH₄OH at 0-5° C. The mixture was stirred at rt for 1 h. The organic layer was decanted, washed with water, dried over MgSO₄, filtered and the solvent was evaporated to give 1.95 g (97%) of intermediate 46b (*S).

### Example A14

### Preparation of Intermediate 47

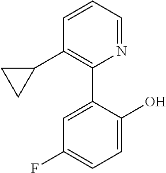 [SMILES: Oc1ccc(F)cc1-c1ncccc1C1CC1]

A mixture of 2-chloro-3-cyclopropylpyridine (5 g; 32.55 mmol), 5-fluoro-2-hydroxyphenylboronic pinacol ester (10.1 mL; 48.82 mmol) and potassium fluoride (9.46 g; 162.75 mmol) in dioxane (125 mL) and water (30 mL). The reaction mixture was degassed and Sphos Pd G2 (469 mg; 0.65 mmol) was added. Then, the reaction was heated at 100° C. for 2 h. The mixture was cooled to RT, then poured into water. EtOAc was added and the reaction mixture was filtered through of pad of celite®. The organic layer was decanted, washed with brine then water, dried over MgSO₄, filtered and evaporated to dryness. The residue was crystallized from Et₂O. The precipitate was filtered and dried to give 6.8 g (91%) of intermediate 47.

### Preparation of Intermediate 48

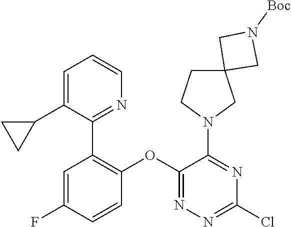 [SMILES: CC(C)(C)OC(=O)N1CC2(CCN(c3nc(Cl)nnc3Oc3ccc(F)cc3-c3ncccc3C3CC3)C2)C1]

To a solution of intermediate 47 (5.6 g, 15.7 mmol) in THE (180 mL) were added intermediate 30 (3.6 g, 15.7 mmol) and DBU (4.9 mL, 33 mmol). After stirring for 72 h at room temperature, the reaction mixture was quenched with water and extracted with EtOAc. The combined organic layers were dried with Na₂SO₄, filtered and concentrated under reduced pressure. The residue was purified by flash chromatography column (mobile phase: gradient from 0.1% NH₄OH, 1% MeOH, 99% DCM to 0.3% NH₄OH, 3% MeOH, 97% DCM) to give 6.4 g (74%) of the intermediate 48.

### Preparation of Intermediate 49

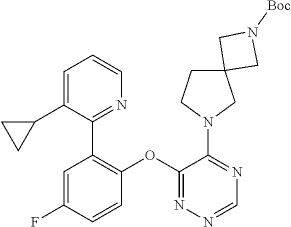 [SMILES: CC(C)(C)OC(=O)N1CC2(CCN(c3ncnnc3Oc3ccc(F)cc3-c3ncccc3C3CC3)C2)C1]

Intermediate 48 (6.4 g, 11.58 mmol) in THE (300 mL) were added 1,1′-bis(diphenylphosphino)ferrocene-palladium(II)dichloride dichloromethane complex (956 mg, 1.16 mmol), sodium borohydride (875 mg, 24 mmol) and N,N,N′,N′-tetramethylethylenediamine (3.5 mL, 23.14 mmol). After stirring overnight at room temperature, the reaction mixture was quenched with water and extracted with EtOAc. The combined organic layers were dried with Na₂SO₄, filtered and concentrated under reduced pressure. The residue was purified by flash chromatography column (mobile phase from 0.1% NH₄OH, 1% MeOH, 99% DCM) to give 3 g (50% yield) of the intermediate 49.

### Preparation of Intermediate 50

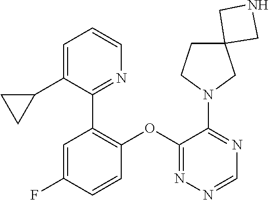 [SMILES: Fc1ccc(Oc2nncnc2N2CCC3(CNC3)C2)c(-c2ncccc2C2CC2)c1]

At 0° C., TFA (8.9 mL; 73.28 mmol) was added to a solution of intermediate 49 (3 g; 5.78 mmol) in DCM (90 mL) and the reaction mixture was stirred for 18 hours at room temperature. The reaction was concentrated under vacuum. The residue was dissolved in 40 mL of water, the solution was basified with 15% aqueous solution of NH₄OH. The aqueous layer was extracted with DCM (*3). The organic layer was decanted, washed again with brine, dried over MgSO₄, filtered and evaporated to dryness to give 2.4 g of intermediate 50 which was used in the next step without further purification.

The intermediates reported below were prepared following an analogous methodology as described for intermediate 50 starting from the corresponding intermediates:

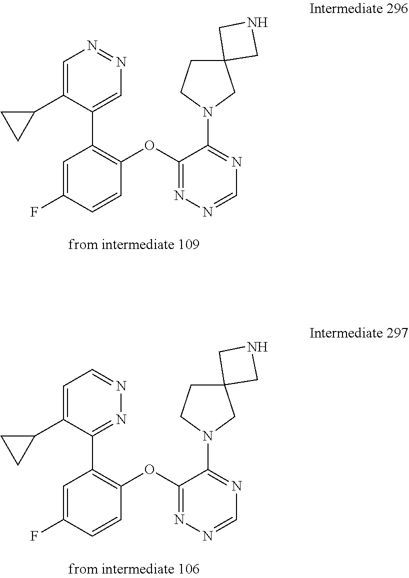 [SMILES: Fc1ccc(Oc2nncnc2N2CCC3(CNC3)C2)c(-c2cnncc2C2CC2)c1 ; Fc1ccc(Oc2nncnc2N2CCC3(CNC3)C2)c(-c2nnccc2C2CC2)c1]

### Preparation of Intermediate 51, 51a (*R), 51b (*S

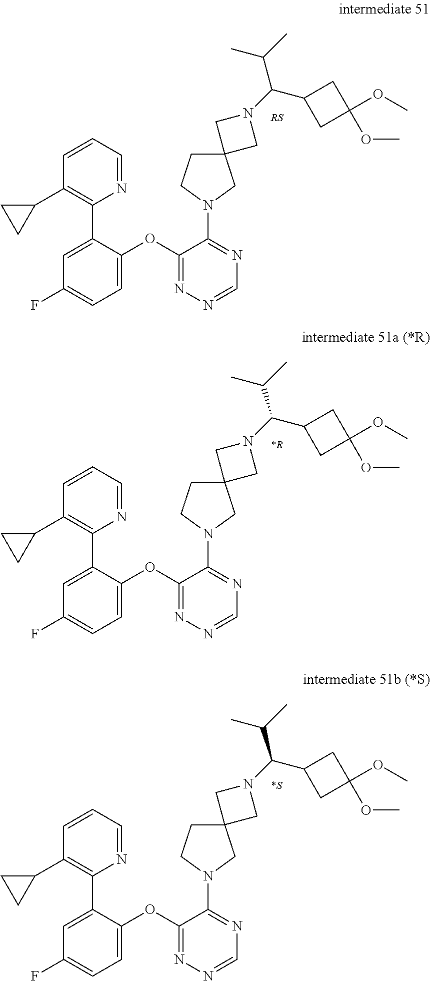 [SMILES: COC1(OC)CC(C(C(C)C)N2CC3(CCN(c4ncnnc4Oc4ccc(F)cc4-c4ncccc4C4CC4)C3)C2)C1 ; COC1(OC)CC([C@@H](C(C)C)N2CC3(CCN(c4ncnnc4Oc4ccc(F)cc4-c4ncccc4C4CC4)C3)C2)C1 ; COC1(OC)CC([C@H](C(C)C)N2CC3(CCN(c4ncnnc4Oc4ccc(F)cc4-c4ncccc4C4CC4)C3)C2)C1]

A mixture of intermediate 50 (1.3 g; 3.1 mmol), intermediate 36 (0.752 g; 4.08 mmol), AcOH (178 μL; 3.11 mmol) and NaBH₃CN (0.29 g; 4.66 mmol) in MeOH (50 mL) was stirred at 50° C. overnight. The reaction mixture was poured onto a 10% aqueous solution of K₂CO₃ and DCM was added. The mixture was extracted with DCM (3×). The organic layer was dried over MgSO₄, filtered and the solvent was evaporated. The crude product was purified by chromatography over silica gel (mobile phase: gradient from 0% NH₄OH, % MeOH, 99% DCM to 0.1% NH₄OH, 5% MeOH, 95% DCM). The pure fractions were collected and the solvent was evaporated. This residue (1.2 g) was purified by chiral SFC (CHIRALPAK AD-H 5 μm 250*30 mm, mobile phase: 70% CO₂, 30% i-PrOH (0.3% iPrNH₂)). The pure fractions were collected and the solvent was evaporated to give 464 mg (25%) of intermediate 51a (*R) (ee100%) and 476 mg (26%) of intermediate 51b (*S) as an off-white (ee 100%) solid.

### Preparation of Intermediate 52a (*R), 52b (*S

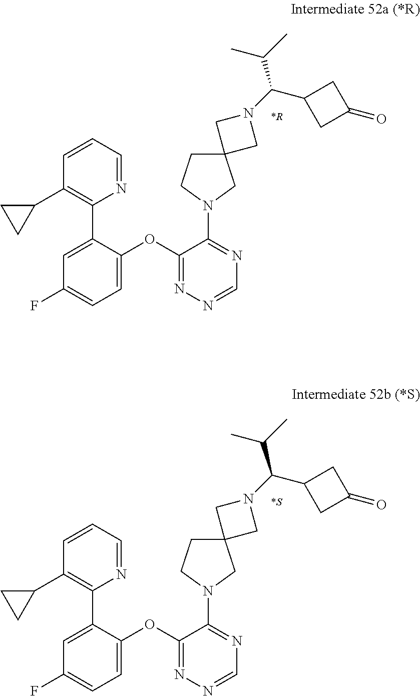 [SMILES: CC(C)[C@H](C1CC(=O)C1)N1CC2(CCN(c3ncnnc3Oc3ccc(F)cc3-c3ncccc3C3CC3)C2)C1 ; CC(C)[C@@H](C1CC(=O)C1)N1CC2(CCN(c3ncnnc3Oc3ccc(F)cc3-c3ncccc3C3CC3)C2)C1]

At 5° C., TFA (1.2 mL; 15.76 mmol) was added dropwise to a solution of intermediate 51a (*R) (464 mg; 0.79 mmol) in DCM (16 mL) and the reaction mixture was stirred for 15 h at rt. The reaction was evaporated to dryness. The residue was dissolved in DCM and basified with a 10% aqueous solution of K₂CO₃. The organic layer was decanted, washed with water, dried over MgSO₄, filtered and the solvent was evaporated to give 400 mg (94%) of intermediate 52a (*R).

At 5° C., TFA (1.2 mL; 15.76 mmol) was added dropwise to a solution of intermediate 51b (*S) (476 mg; 0.81 mmol) in DCM (15 mL) and the reaction mixture was stirred for 15 h at rt. The reaction was evaporated to dryness. The residue was dissolved in DCM and basified with a 10% aqueous solution of K₂CO₃. The organic layer was decanted, washed with water, dried over MgSO₄, filtered and the solvent was evaporated to give 430 mg (98%) of intermediate 52b (*S).

The intermediates reported below were prepared following an analogous methodology as described for intermediate 52a and intermediate 52b starting from the corresponding intermediates:

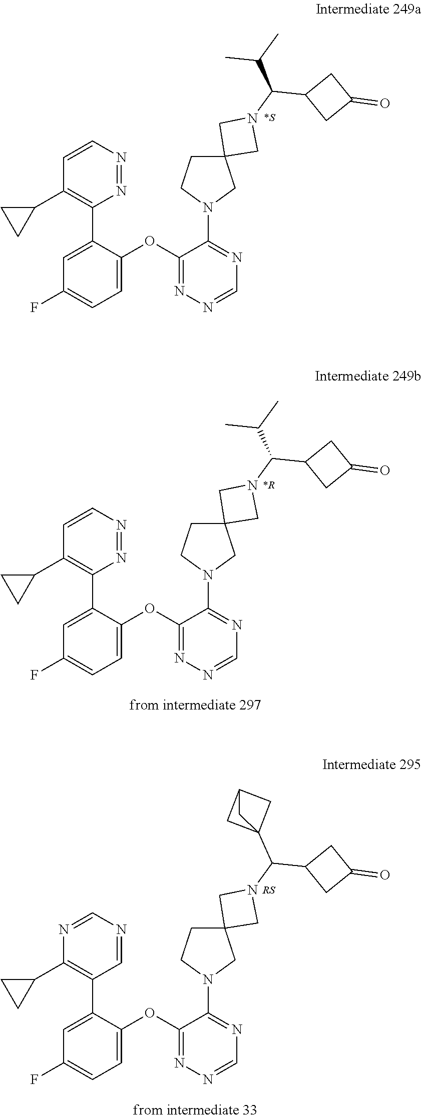 [SMILES: CC(C)[C@@H](C1CC(=O)C1)N1CC2(CCN(c3ncnnc3Oc3ccc(F)cc3-c3nnccc3C3CC3)C2)C1 ; CC(C)[C@H](C1CC(=O)C1)N1CC2(CCN(c3ncnnc3Oc3ccc(F)cc3-c3nnccc3C3CC3)C2)C1 ; O=C1CC(C(N2CC3(CCN(c4ncnnc4Oc4ccc(F)cc4-c4cncnc4C4CC4)C3)C2)C23CC(C2)C3)C1]

### Example A15

### Preparation of Intermediate 53

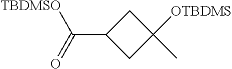 [SMILES: CC1(O[Si](C)(C)C(C)(C)C)CC(C(=O)O[Si](C)(C)C(C)(C)C)C1]

Under nitrogen atmosphere, tert-butylchlorodimethylsilane (2.9 g, 19.3 mmol) and 1H-imidazole (1.66 g, 24.3 mmol) were added to a solution of 3-hydroxycyclobutane-1-carboxylic acid (1.13 g, 9.7 mmol) in THE (15 mL). The reaction mixture was stirred at room temperature overnight. The reaction was filtered to remove insoluble, washing with DCM and, then concentrated in vacuo to give 2.9 g of the intermediate 53 The intermediate was used without any further purification in the next step.

### Preparation of Intermediate 54

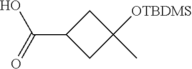 [SMILES: CC1(O[Si](C)(C)C(C)(C)C)CC(C(=O)O)C1]

A solution of K₂CO₃ (141 mg; 1 mmol) in water (2.2 mL) was added to a solution of intermediate 53 (913 mg; 2.54 mmol) in MeOH (6.5 mL) and THE (2.2 mL). The reaction mixture was stirred at room temperature for 4 hours. The solvents were evaporated. The reaction was cooled to 0° C. with an ice bath. Then, an aqueous solution of HCl (1.5 N) was added dropwise until pH<2. The mixture was extracted twice with EtOAc. The combined organic phases were washed with brine, dried over MgSO₄, filtered and concentrated to obtain 493 mg (79%) of intermediate 54 which was directly used in the next step without any further purification

### Preparation of Intermediate 55

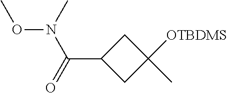 [SMILES: CON(C)C(=O)C1CC(C)(O[Si](C)(C)C(C)(C)C)C1]

A mixture of intermediate 54 (261 mg; 1 mmol), EDCI (307 mg; 1.6 mmol), N,O-dimethylhydroxylamine hydrochloride (156 mg; 1.6 mmol) then, DMAP (6.5 mg; 0.054 mmol) and DIPEA (0.75 mL; 4.3 mmol) in DCM (6 mL) was stirred at rt overnight. The reaction mixture was diluted with DCM (10 mL) and washed with an aqueous solution of HCl (1 N) (2×5 mL), water (10 mL) then with a saturated solution of NaHCO₃ (2×10 mL). The organic layer was separated, dried over MgSO₄, filtered and evaporated to dryness to give 144 mg (45%) of intermediate 55 which was directly used in the next step without any further purification

### Preparation of Intermediate 56

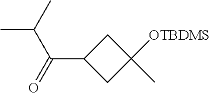 [SMILES: CC(C)C(=O)C1CC(C)(O[Si](C)(C)C(C)(C)C)C1]

Under nitrogen atmosphere and at 0° C., isopropylmagnesium chloride (2.3 mL; 3 mmol, 1.3 M in THF) was added to a solution of intermediate 55 (144 mg; 0.5 mmol) in THE dry (5 mL). The reaction mixture was stirred at 0° C. for 1 hour. Then, the solution was allowed to slowly warm to room temperature and stirred for 2 hours. The reaction mixture was poured into iced water and EtOAc was added. The organic layer was separated, washed with brine, dried over MgSO₄, filtered and evaporated to dryness to give 117 mg (86%) of intermediate 56.

### Preparation of Intermediate 57

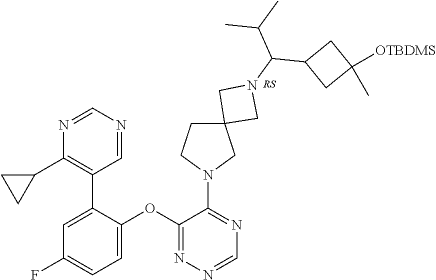 [SMILES: CC(C)C(C1CC(C)(O[Si](C)(C)C(C)(C)C)C1)N1CC2(CCN(c3ncnnc3Oc3ccc(F)cc3-c3cncnc3C3CC3)C2)C1]

A mixture of intermediate 33 (140 mg; 0.33 mmol), intermediate 56 (117 mg; 0.43 mmol), AcOH (19 μL; 0.33 mmol) and NaBH₃CN (47 mg; 0.75 mmol) in MeOH (5 mL) was stirred at 50° C. overnight. The reaction mixture was poured onto a saturated solution of NaHCO₃ and DCM was added. The mixture was extracted with DCM (3×). The organic layer was dried over MgSO₄, filtered and the solvent was evaporated. The crude product (258 mg) was purified by chromatography over silica gel (Mobile phase: Gradient from 99% DCM, 1% MeOH (+10% NH₄OH) to 95% DCM, 5% MeOH (+10% NH₄OH)). The pure fractions were collected and the solvent was evaporated to give 104 mg (46%) of intermediate 57.

### Example A16

### Preparation of Intermediate 59

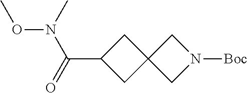 [SMILES: CON(C)C(=O)C1CC2(C1)CN(C(=O)OC(C)(C)C)C2]

To a mixture of 2-(tert-butoxycarbonyl)-2-azaspiro[3.3]heptane-6-carboxylic acid (900 mg; 3.7 mmol) and N,O-dimethylhydroxylamine hydrochloride (400 mg; 4.1 mmol) in DCM (15 mL) was added HATU (2.1 g; 5.6 mmol) and DIPEA (0.96 mL; 5.6 mmol) at room temperature. The resulting mixture was stirred at room temperature for 24 hours. The reaction mixture was poured into water. A saturated aqueous solution of NaHCO₃ and DCM were added. The organic layer was separated, dried over MgSO₄, filtered and the solvent was removed in vacuo. The residue (2.26 g) was purified by chromatography over silica gel (Mobile phase: Gradient from 80% heptane, 20% EtOAc to 40% heptane, 60% EtOAc). The pure fractions were collected, and the solvent was evaporated to give 1 g (100%) of the intermediate 59.

The intermediate 60 reported below was prepared following an analogous methodology starting from 1-boc-1-azaspiro[3.3]heptane-6-carboxylic acid

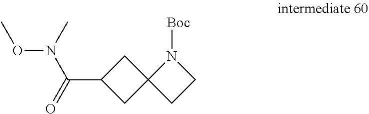 [SMILES: CON(C)C(=O)C1CC2(CCN2C(=O)OC(C)(C)C)C1]

### Preparation of Intermediate 61

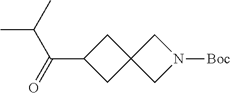 [SMILES: CC(C)C(=O)C1CC2(C1)CN(C(=O)OC(C)(C)C)C2]

Under nitrogen atmosphere and at 0° C., isopropylmagnesium chloride (29 mL; 37.3 mmol, 1.3 M in THF) was added to a solution of intermediate 59 (2.12 g; 7.46 mmol) in THE dry (36 mL). The reaction mixture was stirred at 0° C. for 1 hour. Then, the solution was allowed to slowly warm to room temperature and stirred for 2 hours. The reaction mixture was quenched by 10% aqueous solution of NH₄Cl and EtOAc was added. The organic layer was separated, washed with brine, dried over MgSO₄, filtered and evaporated to dryness. The residue (1.9 g) was purified with chromatography over silica gel (Mobile phase: Gradient from 80% heptane, 20% EtOAc to 40% heptane, 60% EtOAc). The pure fractions were collected, and the solvent was evaporated to give 1.47 g (74%) of the intermediate 61.

The intermediate 62 reported below was prepared following an analogous methodology starting from intermediate 60

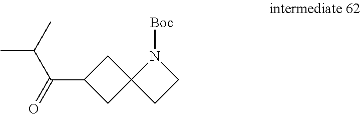 [SMILES: CC(C)C(=O)C1CC2(CCN2C(=O)OC(C)(C)C)C1]

### Example A17

The intermediate 58 reported below was prepared following an analogous methodology as for the preparation of compound 7, starting from intermediate 38b and methyl-3-methylpyrrolidine-3-carboxylate

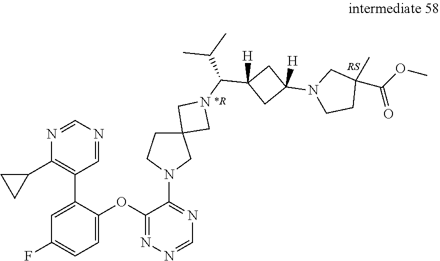 [SMILES: COC(=O)C1(C)CCN([C@H]2C[C@@H]([C@@H](C(C)C)N3CC4(CCN(c5ncnnc5Oc5ccc(F)cc5-c5cncnc5C5CC5)C4)C3)C2)C1]

### Preparation of Intermediate 67

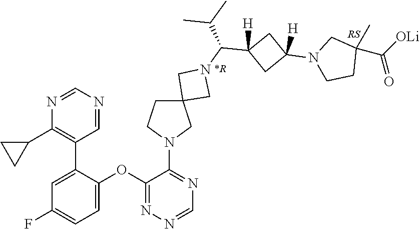

Lithium hydroxyde (101 mg; 2.41 mmol) was added to a solution of intermediate 58 (270 mg; 0.4 mmol) in THE (25 mL) and water (3 mL). The mixture was stirred at rt overnight and evaporated to dryness. The crude was then taken-up with Et₂O and filtered to give, 280 mg of intermediate 67 which was directly used in the next step without any further purification.

### Example A18

### Preparation of Intermediate 68

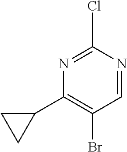 [SMILES: Clc1ncc(Br)c(C2CC2)n1]

(NH₄)₂S₂O₈ (15 g; 65.73 mmol) and AgNO₃ (8.5 g; 50 mmol) were added to water (150 mL), cyclopropanecarboxylic acid (2.1 mL; 26.47 mmol) was added followed by 5-bromo-2-chloropyrimidine (5 g; 25.85 mmol) and CH₃CN (150 mL). The reaction mixture was stirred at room temperature for 72 hours, quenched by slow addition of iced water. EtOAc was added followed by a saturated NaCl solution. The solution was filtered through a Celite® layer and then, extracted with EtOAc (2×500 mL), dried over MgSO₄, filtered and concentrated. The residue (5.83 g) was purified by chromatography over silica gel (Mobile phase: 40% DCM, 60% heptane). The pure fractions were collected, and the solvent was evaporated till dryness to give 3.05 g (51%) of intermediate 68

### Preparation of Intermediate 69

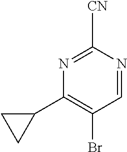 [SMILES: N#Cc1ncc(Br)c(C2CC2)n1]

At rt, TBACN (1.75 g; 6.52 mmol) and DABCO (0.72 g; 6.42 mmol) were added to a solution of intermediate 68 (1 g; 4.28 mmol) in MeCN (20 mL) and the solution was stirred at rt for 2 hours. The solution was poured into cooled water and the product was extracted with EtOAc. The organic layer was separated, dried over MgSO₄, filtered and evaporated to dryness. The residue (1.6 g) was purified by chromatography over silica gel (Mobile phase Gradient from 0% DCM, 100% heptane to 30% DCM, 70% heptane). The pure fractions were collected, and the solvent was evaporated till dryness to give 860 mg (90%) of intermediate 69.

### Preparation of Intermediate 70

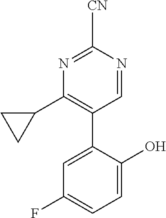 [SMILES: N#Cc1ncc(-c2cc(F)ccc2O)c(C2CC2)n1]

To a previously degassed mixture of intermediate 69 (860 mg; 3.84 mmol), 5-fluoro-2-hydroxyphenylboronic pinacol ester (1.3 g; 5.46 mmol) and potassium fluoride (1.1 g; 18.93 mmol) in 1,4-dioxane (20 mL) were added water (3.9 mL) and SPhos Pd G2 (56 mg; 0.08 mmol). The mixture was heated at 100° C. for 2.30 h in a Schlenk apparatus. The mixture was cooled at room temperature, poured into water. EtOAc was added and the mixture was filtered through of pad of celite®. The organic layer was decanted, washed with brine then water, dried over MgSO₄, filtered and evaporated to dryness. The residue (2.12 g) was purified by chromatography over silica gel (mobile phase: gradient from 100% DCM, 0% MeOH to 98% DCM, 2% MeOH). The pure fractions were collected, and the solvent was evaporated till dryness to give 630 mg (64%) of intermediate 70.

### Preparation of Intermediate 71

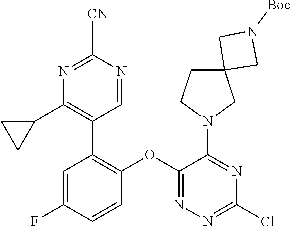 [SMILES: CC(C)(C)OC(=O)N1CC2(CCN(c3nc(Cl)nnc3Oc3ccc(F)cc3-c3cnc(C#N)nc3C3CC3)C2)C1]

A solution of intermediate 70 (2 g; 7.835 mmol), intermediate 30 (2.8 g; 7.77 mmol) and DBU (5.7 mL; 38.94 mmol) in THE (100 mL) was stirred at rt for 24 hours. The solution was poured into cooled water and the product was extracted with EtOAc. The organic layer was separated, dried over MgSO₄, filtered and evaporated to dryness. The residue (5 g) was purified by chromatography over silica gel (Mobile phase: 0.1% NH₄OH, 99% DCM, 1% MeOH). The pure fractions were collected and the solvent was evaporated till dryness. A second purification (3.5 g) was performed via chiral SFC (Stationary phase: CHIRALPAK IC 5 μm 250*30 mm, Mobile phase: 50% CO₂, 50% MeOH). The pure fractions were collected and the solvent was evaporated till dryness yielding 2.6 g (57%) of intermediate 71.

### Preparation of Intermediate 72

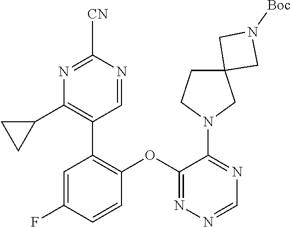 [SMILES: CC(C)(C)OC(=O)N1CC2(CCN(c3ncnnc3Oc3ccc(F)cc3-c3cnc(C#N)nc3C3CC3)C2)C1]

A mixture of intermediate 71 (2.6 g; 4.49 mmol) and TMEDA (1 mL; 6.71 mmol) in dry THE (100 mL) was degassed by N₂ bubbling. Then, Pd(dppf)Cl₂.DCM (415 mg; 0.50 mmol) and NaBH₄ (260 mg; 6.87 mmol) were added. The reaction mixture was stirred at 50° C. overnight in a sealed glassware. The solution was cooled, poured into cooled water and EtOAc was added. The reaction mixture was filtered through a pad of Celite®. The product was extracted with EtOAc. The organic layer was dried over MgSO₄, filtered and evaporated to dryness. The residue was purified by chromatography over silica gel (Mobile phase: Gradient from 100% DCM, 0% MeOH (+10% NH₄OH) to 95% DCM, 5% MeOH (+10% NH₄OH)). The pure fractions were collected, and the solvent was evaporated till dryness yielding 1.84 g (75%) of intermediate 72.

### Preparation of Intermediate 73

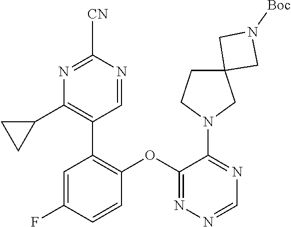 [SMILES: CC(C)(C)OC(=O)N1CC2(CCN(c3ncnnc3Oc3ccc(F)cc3-c3cnc(C#N)nc3C3CC3)C2)C1]

The solution of intermediate 72 (3 g; 5.51 mmol) and TFA (9 mL; 117.5 mmol) in DCM (90 mL) was stirred at rt overnight. The solution was evaporated to dryness and the mixture was poured into cooled water, basified with NH₄OH and the product was extracted with EtOAc. The organic layer was dried over MgSO₄, filtered and evaporated to dryness to afford 2.49 g of intermediate 73 which was used directly for the next step.

### Preparation of Intermediate 74

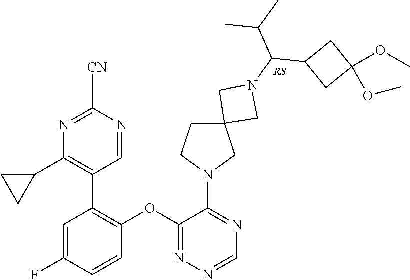 [SMILES: COC1(OC)CC(C(C(C)C)N2CC3(CCN(c4ncnnc4Oc4ccc(F)cc4-c4cnc(C#N)nc4C4CC4)C3)C2)C1]

Under N₂, at rt, to a mixture of intermediate 73 (2 g; 4.5 mmol), intermediate 36 (1.1 g; 5.91 mmol) and AcOH (260 μL; 4.55 mmol) in MeOH (60 mL) was added NaBH₃CN (424 mg; 6.75 mmol) and the reaction was heated at 60° C. overnight. The reaction mixture was cooled, poured onto a mixture of 10% aqueous solution of K₂CO₃ and EtOAc. The mixture was extracted with EtOAc (3×). The organic layer was dried over MgSO₄, filtered and the solvent was evaporated. The residue was purified by chromatography over silica gel (2.6 g) (Mobile phase: Gradient from 100% DCM, 0% MeOH (+10% NH₄OH) to 95% DCM, 5% MeOH (+10% NH₄OH)). The pure fractions were collected and the solvent was evaporated till dryness yielding 450 mg (16% on two steps) of intermediate 74.

### Preparation of Intermediate 75

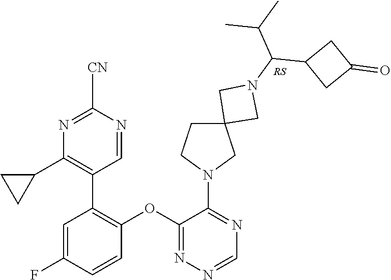 [SMILES: CC(C)C(C1CC(=O)C1)N1CC2(CCN(c3ncnnc3Oc3ccc(F)cc3-c3cnc(C#N)nc3C3CC3)C2)C1]

At 5° C., TFA (1.2 mL; 15.7 mmol) was added dropwise to a solution of intermediate 74 (450 mg; 0.73 mmol) in DCM (12 mL) and the reaction mixture was stirred for 3 h at rt. MeCN was added and the solution was evaporated to dryness. The residue was dissolved in EtOAc and basified with a 30% aqueous solution of NH₄OH at 0-5° C. The organic layer was decanted, washed with water, dried over MgSO₄, filtered and the solvent was evaporated to give 414 mg (99%) of intermediate 75 which was used directly for the next step.

### Example A19

### Preparation of Intermediate 76

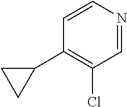

In a 1 L schlenk round bottom flask, cyclopropylzinc bromide 0.5 M in THE (100 mL; 50 mmol) was added dropwise to a previously degassed solution of 4-bromo-3-chloropyridine (6.41 g; 33.33 mmol) and Pd(PPh₃)₄ (1.93 g; 1.67 mmol) in THE (200 mL). The reaction was heated at 65° C. for 18 hours. The reaction mixture was cooled to rt, neutralized with 10% aqueous solution of K₂CO₃ and extracted with Et₂O (twice). The organic layer was washed with brine, dried over MgSO₄, filtered and evaporated to dryness. The residue was purified by chromatography over silica gel (irregular SiOH, 80 g; mobile phase: gradient from 10% EtOAc, 90% heptane to 20% EtOAc, 80% heptane). The pure fractions were collected and evaporated to dryness yielding 3.93 g (77%) of intermediate 76.

### Preparation of Intermediate 77

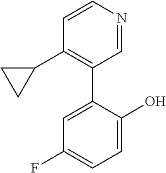 [SMILES: Oc1ccc(F)cc1-c1cnccc1C1CC1]

In a Schlenk round bottom flask, a previously degassed mixture of intermediate 76 (3.91 g; 25.4 mmol), 5-fluoro-2-hydroxyphenylboronic pinacol ester (7.88 g; 33.09 mmol), potassium fluoride (7.39 g; 127 mmol) and SPhos Pd G2 (366 mg; 0.509 mmol) in dioxane (80 mL) and water (27 mL) was refluxed for 3 hours. The reaction mixture was cooled to rt, diluted with EtOAc and poured onto water. The organic layer was decanted, washed with brine, dried over MgSO₄, filtered and evaporated to dryness. The residue was allowed to stand all over the week end. The residue was taken up with DCM and, then, the precipitate was filtered, washed with Et₂O and dried yielding 4.86 g (83%) of intermediate 77.

### Example A21

### Preparation of Intermediate 81

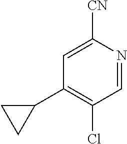 [SMILES: N#Cc1cc(C2CC2)c(Cl)cn1]

In a sealed tube, di-p-iodobis(tri-t-butylphosphino)dipalladium(I) (180 mg; 207 μmol) was added to a mixture of 5-bromo-6-chloro-nicotinonitrile (1.8 g; 8.3 mmol) and a solution of cyclopropylzincbromide 0.5 M in THE (17 mL; 8.7 mmol) in dry THE (34 mL) at room temperature. The reaction mixture was stirred at room temperature for 1 h and quenched with few drops of water. MgSO₄ and celite were added and the solvent were removed under vacuum to give a dry load which was purified by chromatography over silica gel (irregular SiOH, 80 g; mobile phase: gradient from heptane/EtOAc 95:5 to 80:20). The fractions containing product were combined and evaporated in vacuo to give 1.06 g of intermediate 81 (72%) as a brown oil which crystallized upon storage at rt.

### Preparation of Intermediate 82

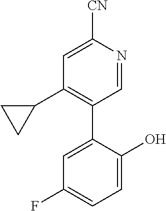 [SMILES: N#Cc1cc(C2CC2)c(-c2cc(F)ccc2O)cn1]

Under N₂ flow, Cs₂CO₃ (7.92 g; 24.3 mmol) followed by Pd(PPh₃)₄ (1.40 g; 1.22 mmol) were added to a stirred solution of intermediate 81 (2.17 g; 12.1 mmol) and 5-fluoro-2-hydroxyphenyl)boronic acid (4.17 g; 26.7 mmol) in a mixture of water (9.5 mL) and dioxane (28.6 mL). The reaction was degassed with N₂ and was then stirred at 90° C. for 18 h. The reaction mixture was cooled to rt, diluted with EtOAc and water was added. The organic layer was decanted, washed with brine, dried over MgSO₄, filtered and evaporated to dryness. The residue was purified by chromatography over silica gel (irregular SiOH, 80 g, dry load; mobile phase: gradient from Heptane/EtOAc 95/5 to 70/30). The fractions containing product were combined and evaporated to give 2.41 g (78%) of intermediate 82 as a yellow solid.

### Example A22

### Preparation of Intermediate 83

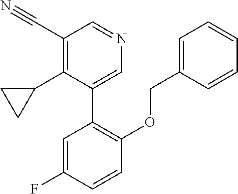 [SMILES: N#Cc1cncc(-c2cc(F)ccc2OCc2ccccc2)c1C1CC1]

In a schlenk, a solution of 5-bromo-4-hydroxy-nicotinonitrile (2.00 g; 10.1 mmol), 2-benzyloxy-5-fluorophenylboronic acid (3.09 g; 12.6 mmol) and K₃PO₄ (3.20 g; 15.1 mmol) in a mixture of dioxane (40 mL) and H₂O (13.3 mL) was purged with nitrogen. CatacXium A Pd G3 (439 mg; 603 μmol) was added. The reaction mixture was purged again with nitrogen and stirred at 80° C. for 17 hours. The reaction mixture was poured in water and extracted twice with a mixture DCM/MeOH (98:2). The organic layers were combined, dried over MgSO₄, filtered and evaporated to dryness. The residue was warmed in i-PrOH (8 mL) and cooled down to room temperature. The precipitate was filtered, washed with diethyl ether and dried in vacuo to give 854 mg of intermediate 83 (27%) as a yellow solid.

### Preparation of Intermediate 84

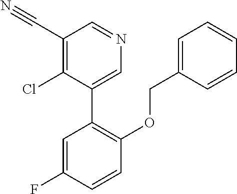 [SMILES: N#Cc1cncc(-c2cc(F)ccc2OCc2ccccc2)c1Cl]

A mixture of intermediate 83 (854 mg; 2.67 mmol) in MeCN (10 mL) was treated with POCl₃ (2.03 mL; 21.9 mmol). The reaction mixture was stirred at 50° C. for 2 hours. The reaction mixture was then cooled down to room temperature, quenched with a 10% aqueous solution of K₂CO₃ and extracted with DCM. The organic layer was dried over MgSO₄, filtered and evaporated in vacuo to give 914 mg (quantitative) of intermediate 84 as a yellow solid.

### Preparation of Intermediate 85

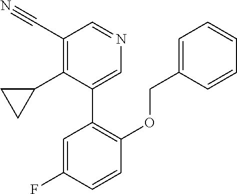 [SMILES: N#Cc1cncc(-c2cc(F)ccc2OCc2ccccc2)c1C1CC1]

A solution of intermediate 84 (914 mg; 2.70 mmol), cyclopropylboronic acid (464 mg; 5.40 mmol) and K₃PO₄ (859 mg; 4.05 mmol) in a mixture of dioxane (11 mL) and H₂O (3.6 mL) was purged with nitrogen. CatacXium A Pd G3 (117 mg; 0.162 mmol) was added. The reaction mixture was purged again with nitrogen and stirred at 80° C. for 4 hours. The reaction mixture was cooled to room temperature and diluted with EtOAc. The organic mixture was washed with water, then with brine, dried over MgSO₄, filtered and the solvent was evaporated in vacuo. The residue was purified by chromatography over silica gel (irregular SiOH, 40 g, dry load; mobile phase: gradient Heptane/EtOAc from 85/15 to 70/30). The fractions containing product were combined and evaporated to give 471 mg (51%) of intermediate 85 as a yellow gummy solid.

### Preparation of Intermediate 86

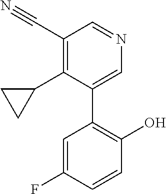 [SMILES: N#Cc1cncc(-c2cc(F)ccc2O)c1C1CC1]

A solution of intermediate 85 (450 mg; 1.31 mmol) and ammonium formate (412 mg; 6.53 mmol) in EtOH (7.6 mL) was treated with Palladium on charcoal (278 mg; 0.131 mmol) and stirred at 75° C. for 45 minutes. The reaction mixture was cooled down to rt, diluted with DCM and filtered through a pad of Celite®. The filtrate was evaporated in vacuo to give a residue which was purified by silica gel chromatography (irregular SiOH, 12 g; mobile phase: gradient DCM/MeOH from 100/0 to 98/2). The fractions containing product were combined and evaporated to give 190 mg of intermediate 86 (57%) as a yellow foam.

### Example A23

### Preparation of Intermediate 87

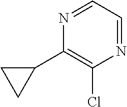

To a mixture of 2,3-dichloropyrazine (5.0 g; 33.562 mmol), 2-cyclopropyl-4,4,5,5-tetramethyl-1,3,2-dioxaborolane (5.64 g; 33.562 mmol) and Pd(amphos)C₁₂ (2.38 g; 3.356 mmol) in 1,4-dioxane (100 mL) was added a solution of sodium carbonate (2 M in water; 50.3 mL). The resulting mixture was stirred for 5 h at 80° C. under an atmosphere of nitrogen. After cooling to room temperature, the reaction mixture was quenched with water and extracted with EtOAc. The combined organic layers were washed with water then brine, dried over Na₂SO₄, filtered and concentrated under reduced pressure. The residue was purified by chromatography over silica gel (PE/EA from 100/0 to 80/20). The fractions containing product were combined and evaporated to give 3.0 g of intermediate 87 (57%) as a colorless oil.

### Preparation of Intermediate 88

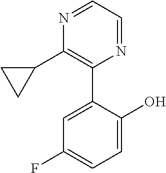 [SMILES: Oc1ccc(F)cc1-c1nccnc1C1CC1]

To a mixture of intermediate 87 (1.21 g; 7.762 mmol) and tetrakis(triphenylphosphine)palladium (370 mg; 0.323 mmol) in 1,4-dioxane (50 mL) was added a solution of sodium carbonate (10 mL; 1 M in water) and the reaction was stirred 3.5 hours at 90° C. under an atmosphere of nitrogen. The reaction mixture was diluted with water and extracted with EtOAc. The combined organic layers were washed with water then brine, dried over anhydrous sodium sulfate, filtered and evaporated to dryness. The residue was purified by chromatography over silica gel (mobile phase: ethyl acetate/petroleum ether 2:1). The fractions containing product were combined and evaporated to give 1.25 g of intermediate 88 (84%) as a light yellow solid.

### Example A24

### Preparation of Intermediate 89

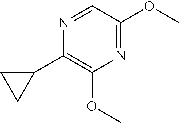

To a stirring solution of 5-bromo-2,4-dimethoxypyrimidine (10.0 g; 45.66 mmol) in 1,4-dioxane were added cyclopropylboronic acid (4.71 g; 65.74 mmol), sodium carbonate (2 M in water, 50 mL) and dichlorobis[di-tert-butyl (4-dimethylaminophenyl)phosphino]palladium(II) (3.23 g; 219.04 mmol). The reaction mixture stirred overnight at room temperature, quenched with water and extracted with EtOAc. The combined organic layers were washed with brine, dried over anhydrous sodium sulfate, filtered and concentrated under reduced pressure. The residue was purified by chromatography over silica gel (mobile phase: PE:EtOAc: 93:7). The fractions containing product were combined and evaporated to give 4.3 g of intermediate 89 (50%) as a colorless oil.

### Preparation of Intermediate 90

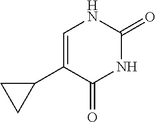 [SMILES: O=c1[nH]cc(C2CC2)c(=O)[nH]1]

To a stirring solution of intermediate 89 (4.8 g; 26.64 mmol) in MeCN (96 mL) was added sodium iodide (12.0 g; 79.91 mmol). The reaction mixture was cooled to 0° C. and chlorotrimethylsilane (8.7 g; 79.91 mmol) was added. After stirring overnight at room temperature, the reaction mixture were quenched with water and stirred for 15 min. The solid was filtered and dried under vacuum to give 3.0 g of intermediate 90 (73% yield) as a yellow solid.

### Preparation of Intermediate 91

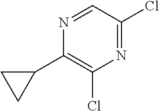 [SMILES: Clc1cnc(C2CC2)c(Cl)n1]

To a stirring solution of intermediate 90 (3.5 g; 23.00 mmol) in phosphorus oxychloride (300 mL) was added N,N-dimethylformamide (0.70 mL). After stirring 2 h at 100° C., the reaction solution was cooled to room temperature and concentrated under reduced pressure. The residue was dissolved in a small amount of DMF and poured into ice water followed by extracting with EtOAc. The combined organic layers were washed with water then brine, dried over Na₂SO₄, filtered and concentrated under reduced pressure to give 4.2 g of intermediate 91 (96%) as a yellow oil.

### Preparation of Intermediate 92

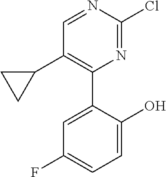 [SMILES: Oc1ccc(F)cc1-c1nc(Cl)ncc1C1CC1]

To a stirring solution of intermediate 91 (5.4 g; 28.56 mmol) in 1,4-dioxane (162 mL) were added (5-fluoro-2-hydroxyphenyl)boronic acid (4.45 g; 28.56 mmol) and tetrakis(triphenylphosphine)palladium (1.65 g; 1.43 mmol) and sodium bicarbonate (2 M in water, 54 mL). After stirring for 2 h at 80° C. under an atmosphere nitrogen, the reaction mixture was quenched with water and extracted with EtOAc. The combined organic layers were washed water, dried over anhydrous sodium sulfate, filtered and concentrated under reduced pressure. The residue was purified by chromatography over silica gel (mobile phase PE:EtOAc 3:1). The fractions containing product were combined and evaporated to give 2.7 g of intermediate 92 (29%, 81% purity evaluated by LCMS) as a light yellow solid.

### Preparation of Intermediate 93

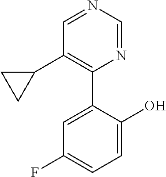 [SMILES: Oc1ccc(F)cc1-c1ncncc1C1CC1]

To a stirring solution of intermediate 92 (1.0 g; 2.08 mmol) in MeOH (55 mL) was added Et₃N (382 mg; 3.78 mmol) and 10% Pd/C (683 mg). After stirring for 30 min under a hydrogen stream (1 atm) at room temperature, the catalyst was filtered off. The filtrate cake was washed with methanol. The collected filtrate was concentrated under reduced pressure to give 550 mg of intermediate 93 (62%).

### Example A25

### Preparation of Intermediate 94

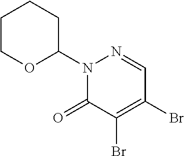 [SMILES: O=c1c(Br)c(Br)cnn1C1CCCCO1]

To a stirring solution of 4,5-dibromopyridazin-3(2H)-one (50 g; 196.95 mmol) in THE (300 mL) was added p-toluenesulfonic acid (3.4 g; 19.694 mmol) and 3,4-dihydro-2H-pyran (82.8 g; 988.72 mmol). After stirring overnight at 60° C., the reaction mixture was quenched with water and extracted EtOAc. The combined organic layer was washed with water then brine, dried over anhydrous sodium sulfate, filtered and concentrated under reduced pressure. The residue was purified by chromatography over silica gel (mobile phase: PE/EtOAc 81/19). The fractions containing product were combined and evaporated to give 65 g of intermediate 94 (83%) as a light yellow solid.

### Preparation of Intermediate 95

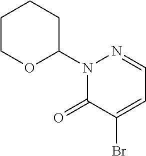 [SMILES: O=c1c(Br)ccnn1C1CCCCO1]

To a stirring solution of intermediate 94 (25 g; 73.97 mmol) in 1,2-dimethoxyethane (200 mL) was added sodium borohydride (5.6 g; 147.93 mmol) at 0° C. After stirring for 18 h at room temperature, the reaction mixture was cooled to 0° C., quenched with water and extracted with ethyl acetate. The combined organic layer was washed with water then brine, dried over anhydrous sodium sulfate, filtered and concentrated under reduced pressure. The residue was purified by chromatography over silica gel (mobile phase: PE/EA 70/30). The fractions containing product were combined and evaporated to give 2.3 g of intermediate 95 (12%) of as a white solid.

### Preparation of Intermediate 96

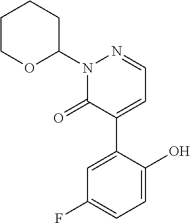 [SMILES: O=c1c(-c2cc(F)ccc2O)ccnn1C1CCCCO1]

To a mixture of intermediate 95 (2.7 g; 10.42 mmol), (5-fluoro-2-hydroxyphenyl)boronic acid (1.6 g; 10.42 mmol) and Pd(PPh₃)₄ (1.2 g; 1.042 mmol) in dioxane (50 mL) was added sodium carbonate solution (20 mL; 2 M in water). After stirring at 90° C. for 5 h, the reaction mixture was cooled to room temperature, diluted with water and extracted with ethyl acetate. The combined organic layers were washed with water then brine, dried over Na₂SO₄, filtered and concentrated under reduced pressure. The residue was purified by chromatography over silica gel (mobile phase: PE/EA: from 100/0 to 80/20). The fractions containing product were combined and evaporated to give 2.46 g of intermediate 96 (78%) as a yellow solid.

### Preparation of Intermediate 97

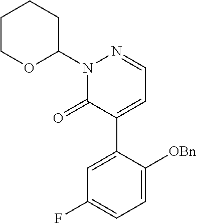 [SMILES: O=c1c(-c2cc(F)ccc2OCc2ccccc2)ccnn1C1CCCCO1]

A mixture of intermediate 96 (2.46 g; 8.47 mmol), benzyl chloride (2 mL; 16.95 mmol) and K₂CO₃ (5.9 g; 42.37 mmol) in acetone (50 mL) was stirred at 60° C. overnight. The mixture was quenched with water and extracted with EtOAc. The combined organic layers were washed with water then brine, dried over Na₂SO₄, filtered and evaporated under reduced pressure. The filtrate was purified by chromatography over silica gel (mobile phase: PE/EA: from 100/0 to 80/20). The fractions containing product were combined and evaporated to give 2.0 g of intermediate 97 (60%) as a yellow oil.

### Preparation of Intermediate 98

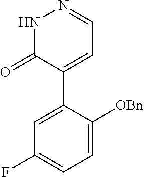 [SMILES: O=c1[nH]nccc1-c1cc(F)ccc1OCc1ccccc1]

A solution of intermediate 97 (2.0 g; 5.26 mmol) in hydrochloric acid (37% in water, 5 mL) and methanol (15 mL) was stirred at 50° C. for 1 h. The solution was concentrated under reduced pressure. The residue was taken up with Et₂O. The precipitate was filtered and dried under vacuum to give 1.0 g of intermediate 98 (64%) as a yellow solid.

### Preparation of Intermediate 99

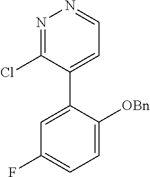 [SMILES: Fc1ccc(OCc2ccccc2)c(-c2ccnnc2Cl)c1]

A solution of intermediate 98 (1 g; 3.38 mmol) in phosphorus oxychloride (15 mL) was stirred at 100° C. for 2 hours. The solution was concentrated under reduced pressure. The residue was quenched with water and extracted with ethyl acetate. The combined organic layers were washed with water then brine, dried over Na₂SO₄, filtered and concentrated under reduced pressure. The residue was purified by chromatography over silica gel (mobile phase: PE/EA from 100/0 to 30/70). The fractions containing product were combined and evaporated to give 600 mg of intermediate 99 (52%) as a yellow oil.

### Preparation of Intermediate 100

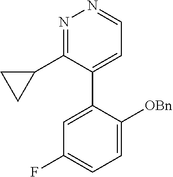 [SMILES: Fc1ccc(OCc2ccccc2)c(-c2ccnnc2C2CC2)c1]

To a solution of intermediate 99 (200 mg; 0.64 mmol) in THE (7.0 mL) were added palladium (II) acetate (14 mg; 0.06 mmol) and 2-dicyclohexylphosphino-2′,6′-dimethoxybiphenyl (52 mg; 0.13 mmol). The resulting mixture was stirred at room temperature for 15 minutes. The reaction mixture was cooled to 0° C. and cyclopropylzinc bromide (0.5 M in THF; 1.9 mL; 0.95 mmol) was added dropwise. After stirring overnight at room temperature, the reaction was quenched with water and extracted with ethyl acetate. The combined organic layers were washed with brine, dried over sodium sulfate, filtered and concentrated under reduced pressure. The residue was purified by chromatography over silica gel (irregular SiOH, 40 g; mobile phase: Petroleum ether:ethyl acetate 70%:30%). The fractions containing product were combined and evaporated to give 150 mg of intermediate 100 (67%) as a light brown oil.

### Preparation of Intermediate 101

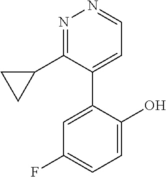 [SMILES: Oc1ccc(F)cc1-c1ccnnc1C1CC1]

To a solution of intermediate 100 (150 mg; 0.47 mmol) in MeOH (10 mL) was added 10% Pd/C (150 mg; 0.14 mmol). After stirring at room temperature under hydrogen atmosphere (2-3 atm.) for 1 h, the reaction mixture was filtered through a diatomite pad. The filtrate was concentrated under reduced pressure to give 100 mg of intermediate 101 (88%) as a light brown solid.

### Example A26

### Preparation of Intermediate 102

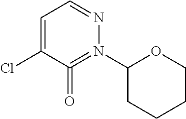

3,4-dihydro-2H-pyran (28 mL; 306.5 mmol) was added at room temperature to a mixture of 4-chloropyridazin-3(2H)-one (10 g; 76.61 mmol) and p-toluenesulfonic acid (1.4 g; 7.67 mmol) in THE (200 mL). The mixture was stirred at 70° C. overnight. After cooling to room temperature, the reaction solution was quenched with water and extracted with ethyl acetate. The combined organic layers were washed with water then brine, dried over Na₂SO₄, filtered and evaporated under reduced pressure. The residue was purified by flash chromatography (mobile phase: PE/EA: from 100/0 to 30/70). The fractions containing product were combined and evaporated to give 16 g of intermediate 102 (84%) as a yellow solid.

### Preparation of Intermediate 103

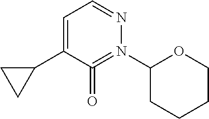

A mixture of intermediate 102 (5 g; 23.29 mmol), cyclopropylboronic acid (2.1 g; 24.46 mmol) and Pd(amphos)Cl₂ (1.65 g; 2.33 mmol) in 1,4-dioxane (75 mL) and 2 M sodium carbonate aqueous solution (25 mL) was stirred at 90° C. for 5 h. After cooling to room temperature, the reaction solution was quenched with water and extracted with ethyl acetate. The combined organic layers were washed with water then brine, dried over Na₂SO₄, filtered and concentrated under reduced pressure. The residue was purified by chromatography over silica gel (mobile phase: PE/EA: from 100/0 to 30/70). The fractions containing product were combined and evaporated to give 3.5 g of intermediate 103 (67%) as a yellow solid.

### Preparation of Intermediate 104

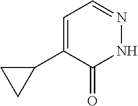

A solution of intermediate 103 (10.0 g; 45.40 mmol) in hydrochloric acid (37% in water, 50 mL) and methanol (150 mL) was stirred at 50° C. for 1 h. The solution was evaporated under reduced pressure. The residue was dissolved in water. The resulting solution was adjusted to pH=7 with NaOH (2 M in water) and extracted with (MeOH/DCM=1/10). The combined organic layers were dried over Na₂SO₄. The solid was filtered off. The filtrate was concentrated under reduced pressure to give 5.2 g of intermediate 104 (76%) as a yellow solid.

### Preparation of Intermediate 105

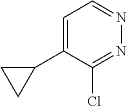

To a solution of intermediate 104 (12.1 g; 88.14 mmol) in acetonitrile (200 mL) was added POCl₃ (41.1 mL; 440.69 mmol). After stirring at 50° C. for 1.5 h, the solution was poured slowly into ice water (200 mL). The resulting solution was adjusted to pH=7 using a saturated aqueous solution of Na₂CO₃ and extracted with ethyl acetate. The combined organic layers were washed with water then brine, dried over Na₂SO₄, filtered and concentrated under reduced pressure. The residue was purified by chromatography over silica gel (PE/EA: from 100/0 to 30/70). The fractions containing product were combined and evaporated to give 9.0 g of intermediate 105 (61%) as a yellow oil.

### Preparation of Intermediate 106

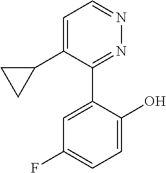 [SMILES: Oc1ccc(F)cc1-c1nnccc1C1CC1]

A mixture of intermediate 105 (9 g; 58.22 mmol), 4-fluoro-2-(4,4,5,5-tetramethyl-1,3,2-dioxaborolan-2-yl)phenol (13.86 g; 58.22 mmol), Pd(PPh₃)₄ (3.36 g; 2.91 mmol) and sodium carbonate solution (43.9 mL; 2 M) in 1,4-dioxane (130 mL) was stirred 90° C. for 3 h. After cooling to room temperature, the reaction mixture was quenched with water and extracted with ethyl acetate. The combined organic layers were washed with water then brine, dried over Na₂SO₄, filtered and evaporated under reduced pressure. The residue was purified by chromatography over silica gel (mobile phase: PE/EA: from 100/0 to 30/70). The fractions containing product were combined and evaporated to give 13 g of intermediate 106 (86%) as a light yellow solid.

### Example A27

### Preparation of Intermediate 107

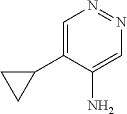

To a solution of 5-bromopyridazin-4-amine (7.7 g; 44.25 mmol) in 1,4-dioxane (130 mL) were added cyclopropylboronic acid (5.7 g; 66.38 mmol), bis-(di-tert-butyl(4-dimethylaminophenyl)phosphine)dichloropalladium(II) (4.7 g; 6.64 mmol) and sodium carbonate solution (2 M in water; 66.4 mL; 132.8 mmol). The resulting mixture was stirred at 90° C. for 36 hours under nitrogen atmosphere. After cooling to room temperature, the reaction was quenched with water and extracted with ethyl acetate. The combined organic layers were washed with brine, dried over sodium sulfate, filtered and concentrated under reduced pressure. The residue was purified by chromatography over silica gel (120 g; mobile phase: dichloromethane/methanol 95%/5%). The fractions containing product were combined and evaporated to give 2.5 g of intermediate 107 (39%) as a red oil.

### Preparation of Intermediate 108

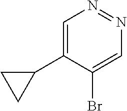

To a solution of intermediate 107 (2.5 g; 18.50 mmol) in acetonitrile (50 mL) were added cupric bromide (3.31 g; 14.80 mmol) and isoamyl nitrite (2.73 mL; 20.35 mmol). The resulting mixture was stirred at 70° C. for 2.5 hours under nitrogen atmosphere. After cooling to room temperature, the reaction was quenched with water and extracted with ethyl acetate. The combined organic layers were washed with brine, dried over sodium sulfate, filtered and concentrated under reduced pressure. The residue was purified by chromatography over silica gel (60 g; mobile phase: Petroleum ether/ethyl acetate 50/50). The fractions containing the product were combined and evaporated to give 1.6 g of intermediate 108 (42%) as a yellow oil.

### Preparation of Intermediate 109

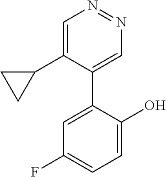 [SMILES: Oc1ccc(F)cc1-c1cnncc1C1CC1]

To a solution of intermediate 108 (1.6 g; 8.04 mmol) in 1,4-dioxane (24 mL) were added (5-fluoro-2-hydroxyphenyl)-boronic acid (1.38 g; 8.84 mmol), bis-(di-tert-butyl(4-dimethylaminophenyl)phosphine)dichloropalladium(II) (570 mg; 0.80 mmol) and sodium carbonate solution (2 M in water; 12.1 mL; 24.11 mmol). The resulting mixture was stirred at 90° C. for 16 hours. After cooling to room temperature, the reaction was quenched with water and extracted with ethyl acetate. The combined organic layers were washed with brine, dried over sodium sulfate, filtered off and concentrated under reduced pressure. The residue was purified by chromatography over silica gel (irregular SiOH, 60 g; mobile phase: PE/EA: 100/0 to 0/100). The fractions containing product were combined and evaporated to give 1.3 g of intermediate 109 (62%) as a red solid.

### Example A28

### Preparation of Intermediate 110

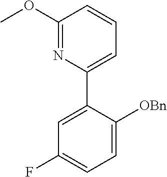 [SMILES: COc1cccc(-c2cc(F)ccc2OCc2ccccc2)n1]

To a stirred solution of 2-bromo-6-methoxypyridine (16.8 g; 89.35 mmol) in 1,4-dioxane (450 mL) were added (2-(benzyloxy)-5-fluorophenyl)boronic acid (22 g; 89.35 mmol), Pd(PPh₃)₄ (5.1 g; 34.69 mmol) and sodium carbonate (168 mL; 2 M). The reaction mixture was stirred for 2 h at 90° C. under an atmosphere of nitrogen, quenched with water and extracted with ethyl acetate. The combined organic layers were washed water, then brine, dried over anhydrous sodium sulfate, filtered and evaporated under reduced pressure. The residue was purified by chromatography over silica gel (eluting system: PE:EA 98:2). The fractions containing product were combined and evaporated to give 26 g of intermediate 110 (94%) as a colorless oil.

### Preparation of Intermediate 111

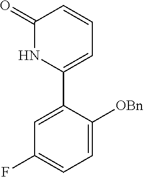 [SMILES: O=c1cccc(-c2cc(F)ccc2OCc2ccccc2)[nH]1]

To a stirring solution of intermediate 110 (23.0 g; 74.353 mmol) in acetonitrile (400 mL) was added p-toluenesulfonic acid monohydrate (17.0 g; 89.24 mmol) and lithium iodide (20.0 g; 148.71 mmol). After stirring 1 h at 80° C., the reaction mixture was quenched with water and extracted with ethyl acetate. The combined organic layers were washed brine, dried over anhydrous sodium sulfate, filtered and evaporated under reduced pressure. The residue was purified by chromatography over silica gel (eluting system: PE:EA 50:50). The fractions containing product were combined and evaporated to give 23.0 g. (90%) of intermediate 111 as a grey solid.

### Preparation of Intermediate 112

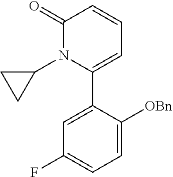 [SMILES: O=c1cccc(-c2cc(F)ccc2OCc2ccccc2)n1C1CC1]

To a stirred solution of intermediate 111 (6.0 g; 20.32 mmol) in acetonitrile (60 mL) were added potassium cyclopropyltrifluoroborate (9,0 g; 60.9 mmol), cupric acetate (923 mg; 5.08 mmol) and o-phenanthroline (458 mg; 2.540 mmol) and potassium carbonate (5.6 g; 40.64 mmol) and water (18 mL). After stirring overnight at 70° C. under an atmosphere of oxygen, the reaction mixture was quenched with water and extracted with ethyl acetate. The combined organic layers was dried over anhydrous sodium sulfate filtered and concentrated under reduced pressure. The residue was purified by silica gel column chromatography, eluted with (PE:EA: 35:65). The fractions containing product were combined and evaporated to give 2.9 g (39%) of intermediate 112 as a off-white solid.

### Preparation of Intermediate 113

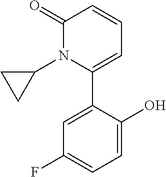 [SMILES: O=c1cccc(-c2cc(F)ccc2O)n1C1CC1]

To a stirred solution of intermediate 112 (1.0 g, 2.98 mmol) in acetonitrile (15 mL) was added iodotrimethylsilane (17.9 g, 89.45 mmol). After stirring overnight at room temperature, the reaction mixture was quenched with saturated sodium bicarbonate and extracted with ethyl acetate. The combined organic layers were washed water, then brine, dried over anhydrous sodium sulfate, filtered and evaporated to dryness under reduced pressure. The residue was purified by chromatography over silica gel (DCM:MeOH 96:4). The fractions containing product were combined and evaporated to give 630 mg (83%) of intermediate 113 as a dark brown solid.

### Example A29 (all Remaining Intermediates

### Preparation of the Intermediate 114

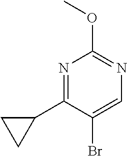

To a solution of 5-bromo-2-methoxypyrimidine (23.8 g, 0.13 mol) in diethyl ether (950 mL) and THE (170 mL) was added cyclopropylmagnesium bromide (133 mL, 0.13 mol, 1 M in THF) at 0° C. After stirring at room temperature for 1 h, the resulting mixture was quenched with water (2.3 mL, 0.13 mol) and followed by the addition of 2,3-dichloro-5,6-dicyano-1,4-benzoquinone (28.6 g, 0.13 mol, dissolved in 70 mL of tetrahydrofuran). The resulting mixture was stirred at room temperature overnight, quenched with water and extracted with EtOAc. The combined organic layers were washed with brine and dried over sodium sulfate, filtered and concentrated under reduced pressure. The residue was purified by chromatography over silica gel (mobile phase: EtOAc/hexanes (1/10)). The pure fractions were collected and evaporated to dryness to give 12.0 g (40%) of desired intermediate 114 as a yellow solid.

### Preparation of Intermediate 115

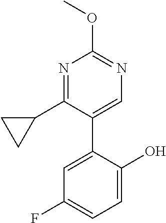 [SMILES: COc1ncc(-c2cc(F)ccc2O)c(C2CC2)n1]

To a solution of intermediate 114 (2.0 g, 8.73 mmol) in 1,4-dioxane (100 mL) were added (5-fluoro-2-hydroxyphenyl)boronic acid (1.6 g, 10.48 mmol), tetrakis(triphenylphosphine)palladium (500 mg, 0.44 mmol) and sodium carbonate solution (17.5 mL, 1 M in water, 17.5 mmol). After stirring for 2 hours at 90° C., the reaction mixture was cooled to room temperature, quenched with water and extracted with EtOAc. The organic layers were combined, washed with brine, dried over sodium sulfate, filtered and concentrated under reduced pressure. The residue was purified by flash chromatography over silica gel (Mobile phase: EtOAc/hexane, 2/3). The pure fractions were collected and evaporated to dryness to give 1.4 g (63%) of the desired intermediate 115 as a light yellow solid.

### Preparation of Intermediate 116

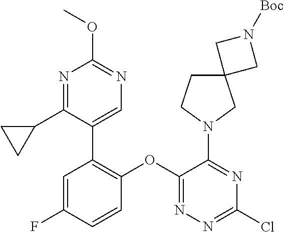 [SMILES: COc1ncc(-c2cc(F)ccc2Oc2nnc(Cl)nc2N2CCC3(CN(C(=O)OC(C)(C)C)C3)C2)c(C2CC2)n1]

To a solution of intermediate 115 (1.5 g, 5.76 mmol) in THE (45 mL) were added intermediate 30 (2.1 g, 5.76 mmol) and DBU (877 mg, 5.76 mmol). The resulting solution was stirred for 48 hours at room temperature, then quenched by water and extracted with EtOAc. The combined organic layers were washed with brine and dried over sodium sulfate, filtered and concentrated under reduced pressure. The residue was purified by flash chromatography over silica gel (Mobile phase: ethyl acetate/hexane: 1/1). The pure fractions were collected and evaporated to dryness to give 3.0 g (78%, 87% purity according to LC/MS) of the desired intermediate 116 as a yellow solid.

### Preparation of Intermediate 117

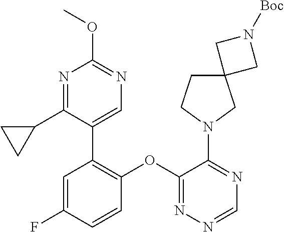 [SMILES: COc1ncc(-c2cc(F)ccc2Oc2nncnc2N2CCC3(CN(C(=O)OC(C)(C)C)C3)C2)c(C2CC2)n1]

To a solution of intermediate 116 (2.9 g, 5.0 mmol) in MeOH (175 mL) was added palladium on activated carbon (10% palladium on activated carbon, 67% moisture) (1.6 g, 1.49 mmol). After stirring at room temperature under a hydrogen atmosphere (1 atm.) for 1 h, the resulting mixture was filtered through a diatomite pad. The filtrate was concentrated under reduced pressure to give 2.7 g (96%) of the desired intermediate 117 as a yellow solid.

### Preparation of Intermediate 118

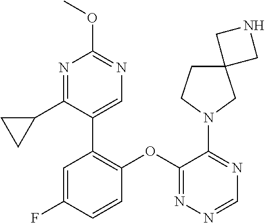 [SMILES: COc1ncc(-c2cc(F)ccc2Oc2nncnc2N2CCC3(CNC3)C2)c(C2CC2)n1]

To a solution of intermediate 117 (2.2 g, 4.0 mmol) in DCM (70 mL) was added TFA (24 mL) at 0° C. The resulting solution was stirred for 1 h at room temperature. The resulting mixture was concentrated under reduce pressure. The residue was diluted with water and the pH was adjusted to 9 with NaOH solution (1 M in water). The resulting solution was extracted with DCM eight times. The combined organic layers were dried over anhydrous Na₂SO₄, filtered and concentrated under reduced pressure to give 1.6 g (81%) of desired intermediate 118 as a white solid.

### Preparation of Intermediate 232

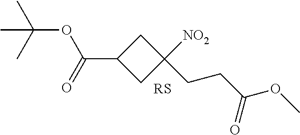 [SMILES: COC(=O)CCC1([N+](=O)[O-])CC(C(=O)OC(C)(C)C)C1]

To a solution of tert-butyl 3-nitrocyclobutanecarboxylate (1.00 g, 4.72 mmol) (synthesis refer to US20170283406A1) and methyl acrylate (0.840 g, 9.76 mmol) in ACN (10 mL) was added DBU (1.45 g, 9.53 mmol) at 0° C., and the mixture was stirred at the same temperature for 20 min. The reaction was quenched with sat. aq. NH₄Cl solution (20 mL) and the mixture was extracted with EtOAc (30 mL×2). The combined organic layers were washed with water (50 mL) and brine (50 mL) and dried over anhydrous Na₂SO₄. After filtration and concentration, the crude residue was purified by FCC (PE:EA=100:0 to 80:20) to afford intermediate 232 (0.8 g, 59% yield) as colorless oil

### Preparation of Intermediate 233

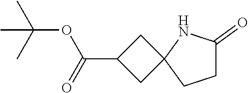 [SMILES: CC(C)(C)OC(=O)C1CC2(CCC(=O)N2)C1]

To a mixture of intermediate 232 (1.58 g, 5.50 mmol) and Nickel(II) chloride hexahydrate (1.2 g, 5.05 mmol) in MeOH (40 mL) was slowly added NaBH₄ (0.95 g, 25.1 mmol) at −10° C. in 3 portions. The mixture was stirred at the same temperature for 3 h. The reaction was quenched with aq. K₂CO₃ solution (0.416 g/mL) at 0° C. The resulting mixture was stirred at 0° C. for 3 h and was further stirred at RT for another 2 h. The mixture was passed through a Celite® pad and the filtrate was concentrated in vacuo to afford intermediate 233 (0.87 g, crude), which was used directly in next step without further purification.

### Preparation of Intermediate 234

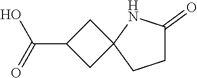 [SMILES: O=C1CCC2(CC(C(=O)O)C2)N1]

The solution of intermediate 233 (0.5 g, 2.22 mmol) in HCl/dioxane (7 mL, 4 M) was stirred at RT for 12 h. The mixture was concentrated in vacuo to afford intermediate 234 (350 mg, crude) as a white solid, which was used directly in next step without further purification.

### Preparation of Intermediate 250

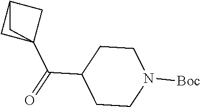 [SMILES: CC(C)(C)OC(=O)N1CCC(C(=O)C23CC(C2)C3)CC1]

To a mixture of bicyclo[1.1.1]pentane-1-carboxylic acid (1.00 g, 8.92 mmol), tert-butyl 4-iodopiperidine-1-carboxylate (4.71 g, 17.8 mmol), 2,2′-bipyridine (696 mg, 4.46 mmol), Nickel(II) acetylacetonate (916 mg, 3.57 mmol), MgCl₂ (2.55 g, 26.8 mmol), zinc powder (4.00 g, 61.2 mmol), 4 Å MS (10.0 g) and DIEA (4.5 mL, 27.2 mmol) in THF/DMF (100 mL/30 mL) was added Boc₂O (7.79 g, 35.7 mmol) under Ar atmosphere at 25° C. After addition, the reaction mixture was stirred at 25° C. for 60 h. The reaction mixture was poured into water (150 mL) and extracted with EtOAc (150 mL×2). The combined layers were washed with brine (200 mL) and dried over anhydrous Na₂SO₄. After filtration and concentration, the residue was purified by column chromatography (EtOAc/PE=0-15%) to afford intermediate 250 (560 mg, 16% yield) as colorless oil.

The intermediate reported below was prepared following an analogous methodology as described for intermediate 250 starting from the corresponding intermediate:

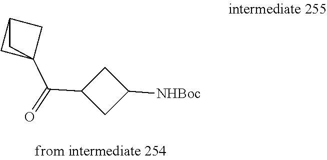 [SMILES: CC(C)(C)OC(=O)NC1CC(C(=O)C23CC(C2)C3)C1]

### Preparation of Intermediate 237

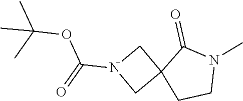 [SMILES: CN1CCC2(CN(C(=O)OC(C)(C)C)C2)C1=O]

NaH (71 mg, 1.8 mmol, 60% in mineral oil) was added to solution of tert-butyl 5-oxo-2,6-diazaspiro[3.4]octane-2-carboxylate (200 mg, 0.884 mmol) in THE (8 mL) cooled at 0° C. under N₂ atmosphere. The reaction mixture was stirred at this temperature for 1 h. Then, Mel (1.48 g, 10.4 mmol) was added dropwise to the reaction mixture at 0° C. and the mixture was slowly warmed to RT and stirred for 2 h. The reaction mixture was quenched with sat. aq. NH₄Cl (10 mL) solution and extracted with EtOAc (20 mL×3). The combined organic layers were washed with brine (5 mL), dried over anhydrous Na₂SO₄. After filtration, the filtrate was concentrated in vacuo to afford intermediate 237 (210 mg, crude) as a brown oil, which was used directly in the next step without further purification.

The intermediates reported below were prepared following an analogous methodology as described for intermediate 237 starting from the corresponding commercial starting materials:

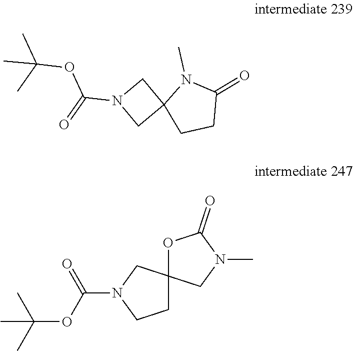 [SMILES: CN1C(=O)CCC12CN(C(=O)OC(C)(C)C)C2 ; CN1CC2(CCN(C(=O)OC(C)(C)C)C2)OC1=O]

### Preparation of Intermediate 238

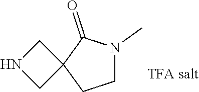

Intermediate 237 (210 mg, 0.874 mmol) was added to a solution TFA (0.5 mL) in DCM (5 mL). The reaction mixture was stirred at RT for 16 h. The reaction mixture was concentrated in vacuo to afford intermediate 238 (300 mg, crude) as a brown oil, which was used directly in the next step without further purification.

The intermediates reported below were prepared following an analogous methodology as described for intermediate 238 starting from the corresponding intermediates or commercial starting materials:

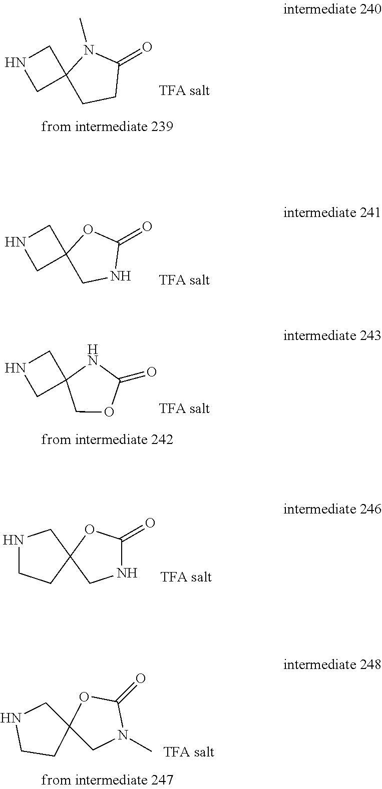

### Preparation of Intermediate 242

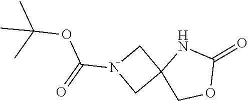 [SMILES: CC(C)(C)OC(=O)N1CC2(COC(=O)N2)C1]

To the mixture of tert-butyl 3-amino-3-(hydroxymethyl)azetidine-1-carboxylate (500 mg, 2.47 mmol) and TEA (1.0 mL, 7.42 mmol) in THE (15 mL) cooled at 0° C. was added a solution of bis(trichloromethyl) carbonate (800 mg, 2.70 mmol) in THE (5 mL) under N₂ atmosphere. The reaction mixture was stirred at 0° C. for 0.5 h and then stirred at RT for additional 3 h. The reaction mixture was poured into sat. aq. NaHCO₃ (30 mL) solution and extracted with EtOAc (20 mL×3). The combined organic layers were washed with brine (30 mL) and dried over anhydrous Na₂SO₄. After filtration, the solvent was removed in vacuo afford intermediate 242 (600 mg, crude) as red solid, which was used directly in next step without further purification.

### Preparation of Intermediate 252

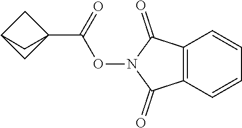 [SMILES: O=C1c2ccccc2C(=O)N1OC(=O)C12CC(C1)C2]

DIC (5.0 g, 39.6 mmol) was added to a solution of bicyclo[1.1.1]pentane-1-carboxylic acid (4.0 g, 35.7 mmol), 2-hydroxyisoindoline-1,3-dione (6.50 g, 39.8 mmol) and DMAP (450 mg, 3.68 mmol) in DCM (100 mL). The resulting mixture was stirred at 25° C. overnight. The reaction mixture was filtered through a pad of Celite® and the filtrate was concentrated in vacuo to give a crude product which was purified by FCC (PE:EtOAc=10:1) to afford intermediate 252 (7.7 g, 84% yield) as white solid.

### Preparation of Intermediate 253

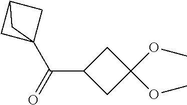

Anhydrous ACN (20 mL) and THF (30 mL) were added to a mixture of intermediate 252 (3.0 g, 11.7 mmol), 3,3-dimethoxycyclobutane-1-carboxylic acid (3.75 g, 23.4 mmol), Ni(BPhen)Cl₂.2DMF (710 mg, 1.16 mmol), zinc powder (2.40 g, 36.7 mmol), benzoic anhydride (5.30 g, 23.4 mmol), MgCl₂ (1.67 g, 17.7 mmol) and LiBr (1.02 g, 11.7 mmol) using a syringe under N₂ atmosphere. The resulting mixture was stirred at 25° C. overnight. The mixture was diluted with EtOAc (200 mL), washed with 1N NaOH (100 mL×2) and brine (50 mL×2), dried over anhydrous Na₂SO₄. After filtration and concentration, the crude product was purified by FCC (PE:EA=10:1) to afford intermediate 253 (1.40 g, 57% yield) as colorless oil.

### Preparation of Intermediate 254

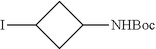 [SMILES: CC(C)(C)OC(=O)NC1CC(I)C1]

To a solution of tert-butyl (3-hydroxycyclobutyl)carbamate (900 mg, 4.81 mmol), 1H-imidazole (982 mg, 14.4 mmol) and Ph₃P (2.52 g, 9.61 mmol) in toluene (15 mL) was added I₂ (1.83 g, 7.21 mmol). The mixture was stirred at 110° C. for 1 h. After cooled to RT, the mixture was diluted with EtOAc (50 mL) and washed with brine (20 mL×2), further dried over anhydrous Na₂SO₄. After filtration and concentration, the crude residue was purified by FCC (PE:EA=5:1) to afford intermediate 254 (620 mg, 43% yield) as white solid.

### Preparation of Intermediate 267

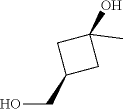

To a suspension of LiAlH₄ (1.17 g, 30.8 mmol) in THF (10 mL) cooled at −10° C. was added a solution of cis-3-hydroxy-3-methylcyclobutanecarboxylic acid (1.00 g, 7.68 mmol) in THF (5 mL) dropwise. The resulting mixture was slowly warmed to 25° C. and stirred for 2 h. The reaction was quenched with water (10 mL). The mixture was filtered through a pad of Celite® and the filtrate was concentrated under reduced pressure. The crude product was purified by FCC over silica gel (PE:EA from 1:0 to 0:1) to afford intermediate 267 (550 mg, 62% yield) as colorless oil.

### Preparation of Intermediate 268

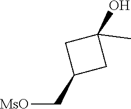

To a solution of intermediate 267 (200 mg, 1.72 mmol) in DCM (10 mL) was added TEA (0.74 mL, 5.3 mmol, 0.73 g/mL) at 0° C. Then MsCl (750 mg, 6.54 mmol) was dropwise added at 0° C. The mixture was slowly warmed to 20° C. and stirred for 1 h. The mixture was washed with water (1 mL) and the organic layer was concentrated under reduced pressure. The crude product was purified by FCC over silica gel (PE:EA from 1:0 to 1:2) to afford intermediate 268 (150 mg, 45% yield) as a colorless oil.

### Preparation of Intermediate 269

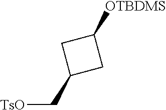 [SMILES: Cc1ccc(S(=O)(=O)OC[C@H]2C[C@@H](O[Si](C)(C)C(C)(C)C)C2)cc1]

To a solution of cis-(3-((tert-butyldimethylsilyl)oxy)cyclobutyl)methanol (500 mg, 2.31 mmol), TEA (1 mL, 7 mmol) and DMAP (57 mg, 0.47 mmol) in DCM (10 mL) cooled at 0° C. was added TsCl (500 mg, 2.62 mmol) in portions. The resulting mixture was slowly warmed to RT and stirred for 12 h. The mixture was poured into H₂O (50 mL) and extracted with DCM (50 mL×3). The combined organic layers were dried over anhydrous Na₂SO₄. After filtration and concentration, the crude product was purified by FCC (PE:EtOAc=1:0 to 10:1) to afford intermediate 269 (700 mg, 82% yield) as a white solid.

The intermediate reported below was prepared following an analogous methodology as described for intermediate 269 starting from the corresponding commercial starting material:

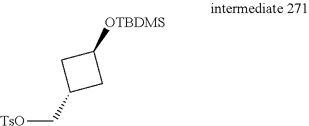 [SMILES: Cc1ccc(S(=O)(=O)OC[C@H]2C[C@H](O[Si](C)(C)C(C)(C)C)C2)cc1]

### Preparation of Intermediate 270

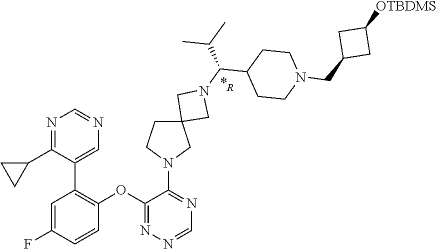 [SMILES: CC(C)[C@H](C1CCN(C[C@H]2C[C@@H](O[Si](C)(C)C(C)(C)C)C2)CC1)N1CC2(CCN(c3ncnnc3Oc3ccc(F)cc3-c3cncnc3C3CC3)C2)C1]

The mixture of compound 3 (600 mg, 1.07 mmol), intermediate 269 (500 mg, 1.35 mmol), K₂CO₃ (230 mg, 1.66 mmol) and KI (36 mg, 0.22 mmol) in ACN (10 mL) was stirred at 90° C. for 16 h. After cooled to RT, the reaction mixture was poured into H₂O (50 mL) and extracted with DCM (50 mL×3). The combined organic layers were dried over anhydrous Na₂SO₄. After filtration and concentration, the crude product was purified by FCC (DCM:MeOH=1:0 to 15:1) to afford intermediate 270 (700 mg, 80% yield) as a white solid.

The intermediate reported below was prepared following an analogous methodology as described for intermediate 270 starting from the corresponding intermediates:

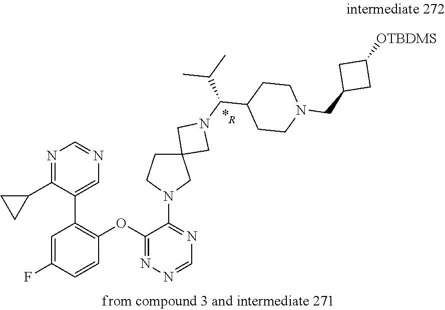 [SMILES: CC(C)[C@H](C1CCN(C[C@H]2C[C@H](O[Si](C)(C)C(C)(C)C)C2)CC1)N1CC2(CCN(c3ncnnc3Oc3ccc(F)cc3-c3cncnc3C3CC3)C2)C1]

### Preparation of Intermediate 273

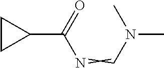

(mixture E/Z not determined)

To a solution of cyclopropanecarboxamide (3.00 g, 35.3 mmol) in toluene (30 mL) was added N,N-dimethylformamide dimethyl acetal (8.40 g, 70.5 mmol). The mixture was stirred at 120° C. for 2 h. After cooled to RT, the mixture was concentrated in vacuo to afford intermediate 273 (5.0 g, crude) as a yellow solid, which was used directly in next step without further purification.

### Preparation of Intermediate 274

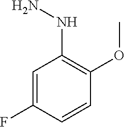

A solution of 5-fluoro-2-methoxyaniline (10.0 g, 70.9 mmol) in 12 M HCl (30 mL) and H₂O (15 mL) was stirred at 0° C. for 20 min. Then a solution of NaNO₂ (6.36 g, 92.2 mmol) in H₂O (15 mL) was slowly added at 0° C. The resulting mixture was slowly warmed to 25° C. and stirred for 1 h. Then SnCl₂ (26.9 g, 142 mmol) in HCl (30 mL) was added at −20° C. and the mixture was stirred at −20° C. for 2 h. The mixture was basified with NaOH (2 M) at −20° C. to adjust the pH value to 12. After slowly warming to RT, the mixture was extracted with DCM (500 mL) and washed with brine (200 mL×3), further dried over anhydrous Na₂SO₄. After filtration, the filtrate was concentrated in vacuo to afford intermediate 274 (7.5 g, crude) as brown oil, which was used directly in next step without further purification.

### Preparation of Intermediate 275

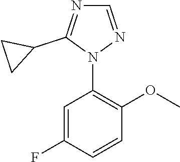 [SMILES: COc1ccc(F)cc1-n1ncnc1C1CC1]

To a solution of intermediate 274 (5.50 g, crude) in AcOH (50 mL) was added intermediate 273 (5.00 g, crude) at 0° C. The resulting mixture was warmed to RT and stirred for 12 h. The mixture was basified with NaOH (2 M) to adjust the pH value to 12 and extracted with EtOAc (100 mL×3). The combined organic layers were washed with brine (100 mL×3) and dried over anhydrous Na₂SO₄. After filtration and concentration, the crude product was purified by FCC over silica gel (PE:EA from 1:0 to 2:1) to afford intermediate 275 (3.0 g) as a brown solid.

### Preparation of Intermediate 276

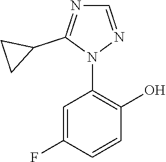 [SMILES: Oc1ccc(F)cc1-n1ncnc1C1CC1]

To a solution of intermediate 275 (3.00 g, 12.9 mmol) in DCM (30 mL) was slowly added BBr₃ (3.60 mL, 38.1 mmol) at −78° C. under N₂ atmosphere. The mixture was stirred at −78° C. for 1 h and further stirred at RT for 12 h. The mixture was basified with NaOH (2 M) to adjust the pH value to 12 and extracted with DCM (200 mL). The organic layer was washed with brine (100 mL×3). The combined aqueous phase was extracted with DCM (100 mL×3) again and the combined organic layers were dried over anhydrous Na₂SO₄. After filtration and concentration, the crude product was purified by FCC over silica gel (PE:EtOAc from 1:0 to 1:1) to afford intermediate 276 (1.90 g, 66% yield) as a brown solid.

The intermediate reported below was prepared following an analogous methodology as described for intermediate 276 starting from the corresponding intermediate:

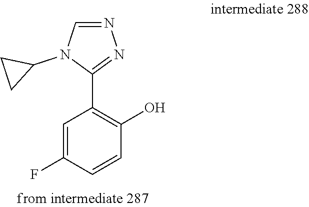 [SMILES: Oc1ccc(F)cc1-c1nncn1C1CC1]

### Preparation of Intermediate 280

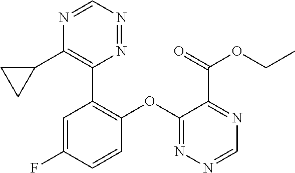 [SMILES: CCOC(=O)c1ncnnc1Oc1ccc(F)cc1-c1nncnc1C1CC1]

Intermediate 279 (450 mg, 1.13 mmol) was dissolved in THE (15 mL), then isoamyl nitrite (0.55 mL, 4.1 mmol) was added. The reaction was heated at 65° C. for 3 h before it was cooled to RT. The reaction mixture was concentrated in vacuo and the crude product was purified by FCC (EA:PE from 1:10 to 1:3) to afford intermediate 280 (200 mg, 46% yield) as yellow oil.

### Preparation of Intermediate 294

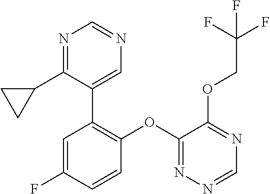 [SMILES: Fc1ccc(Oc2nncnc2OCC(F)(F)F)c(-c2cncnc2C2CC2)c1]

The mixture of intermediate 13 (3.5 g, 9.91 mmol), molecular sieve (6.0 g, 4 Å) and 2,2,2-trifluoroethanol (30 mL) was first purged with Ar gas for three times and stirred at 65° C. for 3 h. Then 1,3-dibromo-1,3,5-triazinane-2,4,6-trione (5.69 g, 19.8 mmol) was added to the mixture at 25° C. and the mixture was further stirred at 65° C. for 8 h. After cooled to RT, the mixture was filtered through a pad of Celite® and concentrated under reduced pressure to give the crude product which was purified by FCC (eluent: PE:EA from 1:0 to 3:1) to afford intermediate 294 (1.8 g, 45% yield) as a yellow oil.

The intermediates reported below were prepared following an analogous methodology as described for intermediate 294 starting from the corresponding intermediates:

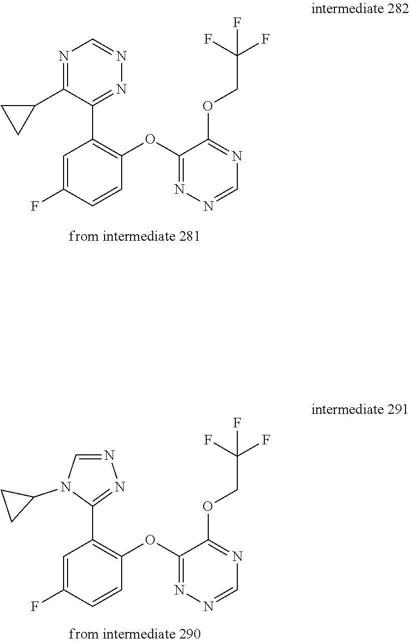 [SMILES: Fc1ccc(Oc2nncnc2OCC(F)(F)F)c(-c2nncnc2C2CC2)c1 ; Fc1ccc(Oc2nncnc2OCC(F)(F)F)c(-c2nncn2C2CC2)c1]

### Preparation of Intermediate 285

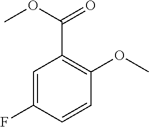

To a solution of 5-fluoro-2-methoxybenzoic acid (10.0 g, 58.8 mmol) in DCM (150 mL) and MeOH (150 mL) cooled at 0° C. was slowly added TMSCH₂N₂ (88.0 mL, 176 mmol, 2 M in hexane). The reaction mixture was slowly warmed to RT and stirred for 2 h. The reaction mixture was concentrated under reduced pressure and the crude product was purified by FCC (PE:EtOAc=10:1 to 3:1) to afford intermediate 285 (12 g, 89% purity, 99% yield) as a yellow oil.

### Preparation of Intermediate 286

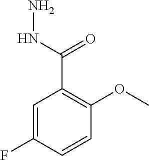

The mixture of intermediate 285 (4.00 g, 21.7 mmol) and hydrazine hydrate (2.02 mL, 65.0 mmol) in EtOH (10 mL) was stirred at 90° C. for 16 h. After cooled to RT, the reaction mixture was concentrated in vacuo to afford intermediate 286 (2.9 g, crude) as a white solid, which was used directly in next step without further purification.

### Preparation of Intermediate 287

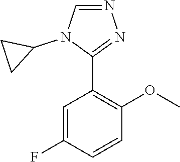 [SMILES: COc1ccc(F)cc1-c1nncn1C1CC1]

To the solution of intermediate 286 (2.80 g, 15.2 mmol) in ACN (60 mL) was added N,N-dimethylformamide dimethyl acetal (1.85 mL, 19.8 mmol) and the reaction mixture was stirred at 50° C. for 1 h. Then cyclopropanamine (5.27 mL, 76.0 mmol) in ACN (10 mL) was added to above mixture and followed with addition of AcOH (1.74 mL, 30.4 mmol). The reaction mixture was further stirred at 120° C. for 16 h. After cooled to RT, the reaction mixture was concentrated and the residue was purified by preparative HPLC (Welch Xtimate C18 150*40 mm*10 μm column, eluent: water (0.2% formic acid)-ACN, from 15% ACN to 45% ACN v/v). The desired fractions were collected and lyophilized to afford intermediate 287 (465 mg, 10% yield) as a white solid.

### Preparation of Intermediate 303

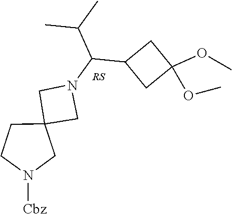 [SMILES: COC1(OC)CC(C(C(C)C)N2CC3(CCN(C(=O)OCc4ccccc4)C3)C2)C1]

To a stirring solution of intermediate 3 (15 g, 60.900 mmol) in methanol (300 mL) was added intermediate 36 (13.61 g, 73.080 mmol) and acetic acid (4.02 g, 66.990 mmol). After stirring for 0.5 hour at room temperature, sodium cyanoborohydride was added (7.65 g, 121.800 mmol). After stirring overnight at 50° C., the reaction mixture was quenched with potassium carbonate solution (10% in water) and extracted with ethyl acetate. The combined organic layers were washed brine and dried over anhydrous sodium sulfate. The solid was filtered off. The filtrate was concentrated under reduced pressure. The residue was purified by silica gel column chromatography, eluted with (EA/PE, 0% EA to 50%) to give 17.8 g (69% yield) of the desired compound as a light yellow oil.

### Preparation of Intermediate 304 and 305

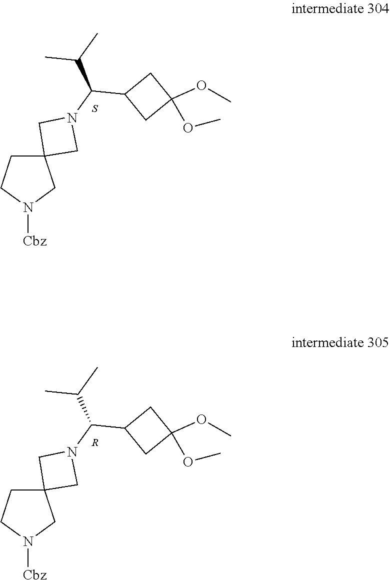

170 g benzyl 2-(1-(3,3-dimethoxycyclobutyl)-2-methylpropyl)-2,6-diazaspiro[3.4]octane-6-carboxylate was purified by SFC with the following conditions: Column: CHIRALPAK IG, 5*25 cm, 10 um; Mobile Phase A: CO₂, Mobile Phase B: EtOH:ACN:DCM=1:1:1; Flow rate: 150 mL/min; Gradient: 40% B; 220 nm; retention time 1=4.45 min; retention time 2=5.88 min; Injection Volumn: 3.8 ml; Number of Runs: 237 to give two fractions.

Fraction A: 67.0 g 39% yield, retention time 1: 5.88 min) of intermediate 304 as light yellow oil.

Fraction B: 65 g (38% yield, retention time 2: 4.45 min) of intermediate 305 as light yellow oil.

### Preparation of Intermediate 306

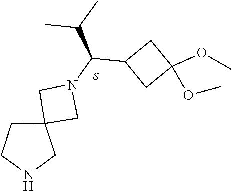

To a solution of intermediate 304 (15 g, 36.010 mmol) in methanol (300 mL) was added palladium on activated carbon (10% palladium) (8 g, 7.517 mmol). Then, the mixture was stirred at room temperature for 5 hours under the hydrogen (2-3 atm.). The mixture was diluted with methanol and filtered through a Celite®. The filtrate was evaporated under reduced pressure to give 9.5 g of intermediate 306 as a yellow oil.

### Preparation of Intermediate 307

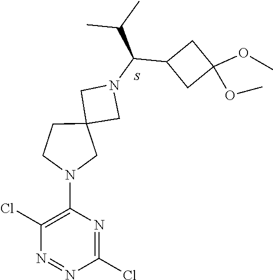

To a solution of 3,5,6-trichloro-1,2,4-triazine (9.4 g, 50.99 mmol) in dichloromethane (100 mL) were added the mixture of intermediate 306 (12.0 g, 42.49 mmol) and triethylamine (12 mL, 84.98 mmol) in dichloromethane (150 mL) under nitrogen at 0° C. After stirring for 3 hours at room temperature under nitrogen, the mixture was quenched with water and extracted with dichloromethane. The combined organic layers were dried over anhydrous sodium sulfate. The solid was filtered off. The filtrate was concentrated under reduced pressure to give 17.3 g (83% yield) of intermediate 307 as a yellow solid.

### Preparation of Intermediate 308

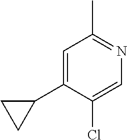

To a solution of 4-bromo-5-chloro-2-methylpyridine (8.8 g, 42.62 mmol) in tetrahydrofuran (90 mL) was added tetrakis(triphenylphosphine)palladium (2.5 g, 2.13 mmol). The resulting mixture was stirred at room temperature for 1 hour under nitrogen atmosphere and, then, cyclopropylzinc(II) bromide (340 mL, 0.5 M in THF) was added. After stirring for 2 hours under nitrogen atmosphere at 65° C., the reaction mixture was quenched with water and extracted with ethyl acetate. The combined organic layers were washed with brine and dried over anhydrous sodium sulfate. The solid was filtered off. The filtrate was concentrated under reduced pressure. The residue was purified by flash chromatography with silica gel 100 g (eluent: petroleum ether-ethyl acetate 75%:25%) to give 7.2 g (97% yield) of intermediate 308 as a yellow solid.

### Preparation of Intermediate 309

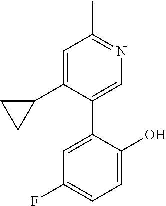 [SMILES: Cc1cc(C2CC2)c(-c2cc(F)ccc2O)cn1]

To a stirring solution of intermediate 308 (7.2 g, 42.95 mmol) in 1,4-dioxane (216 mL) were added (5-fluoro-2-hydroxyphenyl)boronic acid (8.0 g, 51.54 mmol), bis(di-tert-butyl(4-dimethylaminophenyl)phosphine)dichloropalladium (1.5 g, 2.15 mmol) and sodium carbonate aqueous solution (2 M in water, 72 mL). After stirring for 3 hours under nitrogen atmosphere at 100° C., the reaction mixture was cooled down to room temperature, quenched with water and extracted with ethyl acetate. The combined organic layers were washed with brine and dried over anhydrous sodium sulfate. The solid was filtered off. The filtrate was concentrated under reduced pressure. The residue was purified by flash chromatography with silica gel 100 g (eluent: petroleum ether-ethyl acetate 70%:30%) to give 5.8 g (54% yield) of intermediate 309 as a yellow solid.

### Preparation of Intermediate 310

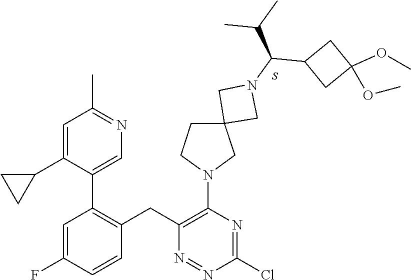

To a solution of intermediate 307 (4.3 g, 9.87 mmol) in tetrahydrofuran (80 mL) were added intermediate 309 (3.0 g, 12.33 mmol) and 1,8-diazabicyclo[5.4.0]undec-7-ene (3.9 g, 25.90 mmol). After stirring at room temperature for 3 days, the reaction mixture was quenched with water and extracted with ethyl acetate. The combined organic layers was dried over anhydrous sodium sulfate. The solid was filtered off. The filtrate was concentrated under reduced pressure. The residue was purified by flash chromatography with silica gel 100 g (eluent: petroleum ether-ethyl acetate 34%:66%) to give 5.0 g (64% yield) of intermediate 310 as a green solid.

### Preparation of Intermediate 311

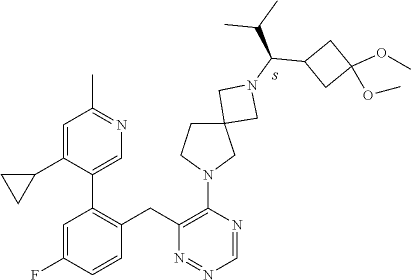

To a solution of intermediate 310 (5.3 g, 8.32 mmol) in tetrahydrofuran (100 mL) were added sodium borohydride (535 mg, 14.14 mmol), N,N,N′,N′-tetramethylethylenediamine (1.6 g, 14.14 mmol) and 1,1′-bis(diphenylphosphino) ferrocene-palladium(II)dichloride dichloromethane complex (680 mg, 0.83 mmol) under nitrogen atmosphere. After stirring at room temperature overnight under nitrogen atmosphere, the reaction mixture was quenched with water and extracted with ethyl acetate. The combined organic layers were dried over anhydrous sodium sulfate. The solid was filtered off. The filtrate was concentrated under reduced pressure. The residue was purified by flash chromatography with silica gel 100 g (eluent: dichloromethane-methanol 93%:7%) to give 4.9 g (88% yield) of intermediate 311 as a brown solid.

### Preparation of Intermediate 312

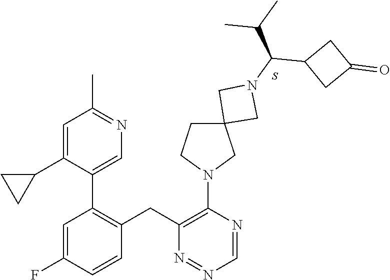

To a solution of intermediate 311 (4.9 g, 8.13 mmol) in acetone (80 mL) were added p-toluenesulfonic acid (7.0 g, 40.65 mmol) and water (40 mL). The resulting mixture was stirred at 65° C. overnight. After cooling down to room temperature, the reaction mixture was quenched with saturated sodium bicarbonate solution and extracted with ethyl acetate. The combined organic layers were dried over anhydrous sodium sulfate. The solid was filtered off. The filtrate was concentrated under reduced pressure. The residue was purified by silica gel chromatography (100 g, eluent: dichloromethane-methanol 98%:2%) to give 4.2 g (88% purity evaluated by LCMS, 81% yield) of intermediate 312 as a yellow solid.

### Preparation of Intermediate 313

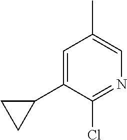

To a solution of 3-bromo-2-chloro-5-methylpyridine (16.0 g, 79.55 mmol) in tetrahydrofuran (160 mL) were added cyclopropylzinc(II) bromide (350.0 mL, 175.000 mmol, 0.5 M in THF) and tetrakis(triphenylphosphine)palladium (4.6 g, 3.98 mmol). After stirring for 10 hours under nitrogen atmosphere at 65° C., the reaction mixture was quenched with water and extracted with ethyl acetate. The combined organic layers were washed with brine and dried over anhydrous sodium sulfate. The solid was filtered off. The filtrate was concentrated under reduced pressure. The residue was purified by flash chromatography with silica gel 320 g (eluent: petroleum ether-ethyl acetate/0%-10%) to give 12 g (82.8% purity evaluated by LC/MS, 75% yield) of intermediate 313 as a colorless oil.

### Preparation of Intermediate 314

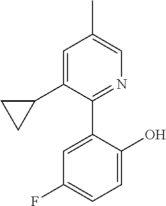 [SMILES: Cc1cnc(-c2cc(F)ccc2O)c(C2CC2)c1]

To a solution of intermediate 313 (15.0 g, 89.48 mmol) in 1,4-dioxane (420 mL) and water (140 mL) were added 5-fluoro-2-hydroxyphenylboronic acid (16.74 g, 107.4 mmol)), sodium carbonate (28.45 g, 268.44 mmol) and tetrakis(triphenylphosphine)palladium(0) (10.34 g, 8.95 mmol). The resulting mixture was stirred at 100° C. for 18 hours under nitrogen. After cooling down to room temperature, the reaction mixture was quenched with water and extracted with ethyl acetate. The organic layers were combined and dried over anhydrous sodium sulfate. The solid was filtered off. The filtrate was concentrated under reduced pressure. The residue obtained was purified by flash chromatography with silica gel 320 g (eluent: petroleum ether-ethyl acetate/0%-100%) to afford a crude product. The crude product was triturated in ethyl acetate/petroleum ether in the ratio of 1:10 to afford 18.0 g (82% yield) of intermediate 314 as an off-white solid.

### Preparation of Intermediate 315

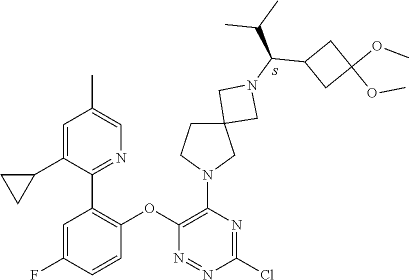

To a solution of intermediate 307 (13.0 g, 30.21 mmol) in tetrahydrofuran (400.0 mL) were added intermediate 314 (8.8 g, 36.25 mmol) and 1,8-diazabicyclo[5.4.0]undec-7-ene (11.0 mL, 75.52 mmol). After stirring at room temperature for 3 days, the reaction mixture was quenched with water and then extracted with ethyl acetate. The organic layers were combined, washed with brine and dried over sodium sulfate. The solid was filtered off. The filtrate was concentrated under reduced pressure. The residue was purified by flash chromatography with silica gel 100 g (eluent: petroleum ether-ethyl acetate/0%-100%) to afford two fractions of intermediate 315.

Fraction A: 8.89 g (97.5% purity evaluated by LCMS; 45% yield) as a white solid.

Fraction B: 2.5 g (88.7% purity, 11% yield) as a yellow solid.

### Preparation of Intermediate 316

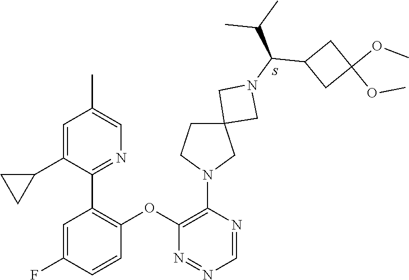

To a solution of intermediate 315 (7.89 g, 12.38 mmol) in tetrahydrofuran (160.0 mL) were added 1,1′-bis(diphenylphosphino)ferrocene-palladium(II)dichloride dichloromethane complex (506 mg, 0.62 mmol), sodium borohydride (796 mg, 21.05 mmol) and N,N,N′,N′-tetramethylethylenediamine (3.2 mL, 21.05 mmol). After stirring overnight at room temperature under nitrogen atmosphere, the reaction mixture was quenched with water and then extracted with ethyl acetate. The organic layers were combined, washed with brine and dried over sodium sulfate. The solid was filtered off. The filtrate was concentrated under reduced pressure. The residue was purified by flash chromatography with silica gel 120 g (eluent: petroleum ether-ethyl acetate: 0%-100%) to afford 6.0 g (81% yield) of intermediate 316 as a yellow solid.

### Preparation of Intermediate 317

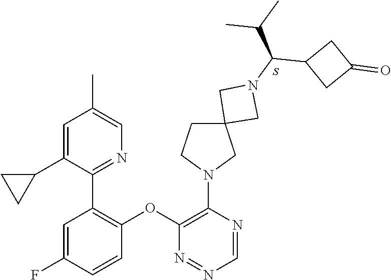

To a solution intermediate 316 (5.4 g, 8.96 mmol) in dichloromethane (26.0 mL) were added trifluoroacetic acid (78.0 mL) at 0° C. The resulting mixture was stirred at room temperature for 5 hours. The solvent was removed under reduced pressure. The residue was quenched with saturated sodium bicarbonate solution and then extracted with dichloromethane for three times. The organic layers were combined, washed with brine and dried over sodium sulfate. The solid was filtered off. The filtrate was concentrated under reduced pressure to give 4.0 g (80% yield) of intermediate 317 as a yellow solid.

### Preparation of Intermediate 318

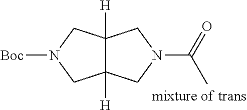 [SMILES: CC(=O)N1CC2CN(C(=O)OC(C)(C)C)CC2C1]

Acetic anhydride (375 mg, 3.67 mmol) was added to a solution consisting of (trans)-tert-butyl hexahydropyrrolo[3,4-c]pyrrole-2(1H)-carboxylate (750 mg, 2.48 mmol), Et₃N (1.0 g, 9.9 mmol) and DCM (20 mL). The reaction mixture was stirred at room-temperature for 6 hours. The reaction mixture was partitioned between H₂O (30 mL) and DCM (30 mL). The aqueous phase was extracted with DCM (20 mL×3) and the combined extracts were dried over anhydrous Na₂SO₄, filtered and concentrated to dryness under reduced pressure to afford 600 mg (95% yield) intermediate 318 (mixture of trans) as a yellow solid.

### Preparation of Intermediate 319

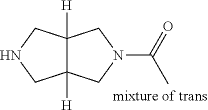

TFA (1.3 mL, 18 mmol) was added to a solution consisting of intermediate 318 (600 mg, 2.36 mmol) in DCM (15 mL). The reaction mixture was stirred at room temperature for 4 hours. The reaction mixture was concentrated under reduced pressure to give the crude product as yellow oil which was dissolved in water (20 mL). The pH of the mixture was adjusted to 10 with NH₃·H₂O and then, lyophilized to afford to 500 mg (crude) of intermediate 319 (mixture of trans) as a yellow solid which was used for next step without further purification.

### Preparation of Intermediate 320

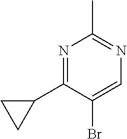

A stir bar, 5-bromo-2-methylpyrimidine (36.0 g, 208 mmol) and dry tetrahydrofuran (250 mL) were added to 2 L three-necked round-bottomed flask before the mixture was cooled to 0° C. under ice-water bath and purged with nitrogen for three times. Then, cyclopropyl magnesium bromide (500 mL, 250 mmol, 0.5 M in THF) was added dropwise to the mixture in 2 hours. The reaction mixture was warmed to room temperature gradually and stirred at room temperature for 1.5 hours. The mixture was cooled to 0° C. under ice-water bath again. A solution of DDQ (47.2 g, 208 mmol) in dry tetrahydrofuran (250 mL) was added to the mixture dropwise in 1.5 hours. The reaction mixture was warmed to room temperature gradually and stirred at room temperature for another 16 hours. 400 mL of EtAOc and 50 mL of sat. NH₄Cl were added to the reaction mixture and stirred for 0.5 h. The reaction mixture was filtered through Celite® and washed with EtOAc (100 mL×3). The organic phase was concentrated under reduced pressure. The residue was purified by FCC (eluent: petroleum ether:ethyl acetate=1:0 to 20:1) to afford the 24.31 g (55% yield) of Intermediate 320 as a yellow oil.

### Preparation of Intermediate 321

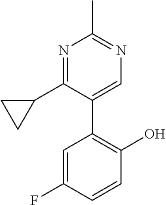 [SMILES: Cc1ncc(-c2cc(F)ccc2O)c(C2CC2)n1]

Pd(dppf)Cl₂ (4.17 g, 5.70 mmol) was added to a mixture of intermediate 320 (24.3 g, 114 mmol), 5-fluoro-2-hydroxyphenyl)boronic acid (21.3 g, 137 mmol), Na₂CO₃ (24.18 g, 228 mmol) in dioxane (300 mL)/H₂O (60 mL). The mixture was stirred at 90° C. for 16 hours under inert atmosphere. The reaction mixture was cooled to room temperature and filtered through a pad of Celite® and washed with EtOAc (50 mL×2). The filtrate was concentrated in vacuo and the residue was dissolved in EtOAc (300 mL). The mixture was washed with brine (50 mL×3), dried over Na₂SO₄, filtered, concentrated in vacuo. The residue was dissolved in EtOAc (30 mL), stirred for 30 min, filtered and washed with EtOAc (10 mL×2). The filter cake was collected and dried to afford 22.3 g (78% yield) of intermediate 321 as a pale solid.

### Preparation of Intermediate 322

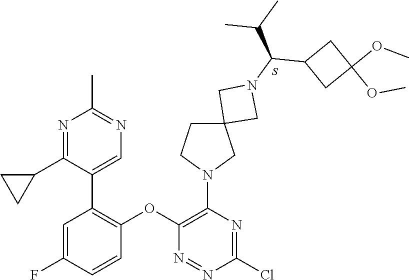

DBU (2.94 g, 19.3 mmol) was added to a solution consisting of intermediate 307 (7.0 g, 16.3 mmol), intermediate 321 (3.98 g, 16.3 mmol) in THF (200 mL). The reaction mixture was stirred at room-temperature for 16 hours. The reaction mixture was partitioned between H₂O (200 mL) and ethyl acetate (200 mL). The aqueous phase was extracted with ethyl acetate (200 mL×3) and the combined extracts were dried over anhydrous Na₂SO₄, filtered and concentrated to dryness under reduced pressure to afford a crude product which was mixed with another crude (2 g) and purified with by FCC (petroleum ether:ethyl acetate=1:0 to 0:1) to yield 7.0 g intermediate 322 (53% yield overall yield based on 8 g of intermediate 307) as a yellow solid.

### Preparation of Intermediate 323

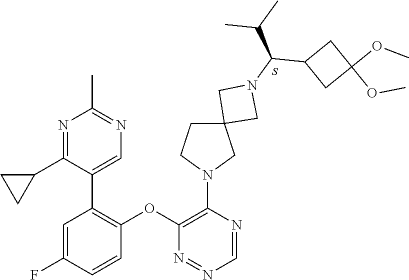

Pd(dppf)Cl₂·DCM (540 mg, 0.661 mmol) was added to a solution of intermediate 322 (6.0 g, 9.4 mmol), NaBH₄ (620 mg, 16.4 mmol), TMEDA (2.1 g, 18 mmol) and THF (150 mL) under N₂. The reaction mixture was stirred at room-temperature for 4 hours. The reaction mixture was partitioned between H₂O (200 mL) and ethyl acetate (300 mL). The aqueous phase was extracted with ethyl acetate (150 mL×3) and the combined extracts were dried over anhydrous Na₂SO₄, filtered and concentrated to dryness under reduced pressure to afford a crude product which was mixed with another crude (1.2 g) and purified by FCC (petroleum ether:ethyl acetate=1:0 to 0:1) to yield 4.5 g (66% yield overall yield from 7 g of intermediate 322) intermediate 323 as a yellow solid.

### Preparation of Intermediate 324

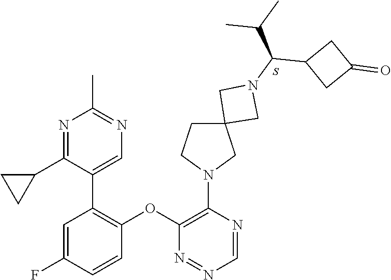

TFA (9.6 mL, 129 mmol) was added to a solution consisting of intermediate 323 (4.0 g, 6.6 mmol) in DCM (100 mL). The reaction mixture was stirred at room-temperature for 4 hours. The mixture was poured into 10% aqueous solution K₂CO₃ (300 mL), and extracted with dichloromethane (200 mL×3). The combined organic extracts were washed with brine (300 mL), aqueous NaHCO₃ (300 mL), H₂O (300 mL), dried over anhydrous Na₂SO₄, filtered, and concentrated to dryness under reduced pressure to afford 3.3 g (84% yield) intermediate 324 as a yellow solid which was used in the next step without further purification.

### Preparation of Intermediate 325

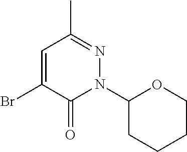

To a solution of 4-bromo-6-methylpyridazin-3(2H)-one (5.00 g, 26.45 mmol) in tetrahydrofuran (100 mL) were added 3,4-dihydro-2H-pyran (9.65 mL, 105.82 mmol) and p-toluenesulfonic acid (455 mg, 2.65 mmol). The resulting mixture was stirred at 70° C. overnight. The reaction was quenched with water and then extracted with ethyl acetate. The organic layers were combined, dried over sodium sulfate, filtered and concentrated under reduced pressure to give a residue. The residue was purified by flash silica gel column chromatography (EA/PE, 0% EA to 20% EA) to afford 4.2 g (52% yield, 89.2% purity based on LCMS) of intermediate 325 as a yellow solid.

### Preparation of Intermediate 326

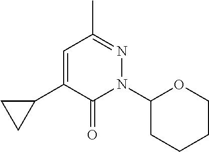 [SMILES: Cc1cc(C2CC2)c(=O)n(C2CCCCO2)n1]

Into a solution of intermediate 325 (8.30 g, 24.62 mmol; 81% purity based on LCMS) in 1,4-dioxane (120 mL) were added cyclopropylboronic acid (2.33 g, 27.08 mmol), Pd(amphos)Cl₂ (871 mg, 1.23 mmol) and sodium carbonate (40 mL, 2 M in water, 80.00 mmol). The resulting mixture was stirred at 90° C. under nitrogen atmosphere overnight. After cooling to room temperature, the reaction was quenched with water and extracted with ethyl acetate. The organic layers were combined, dried over sodium sulfate, filtered and concentrated under reduced pressure to give a residue. The residue was purified by flash silica gel column chromatography (EA/PE, 0% EA to 13% EA) to afford 3.4 g (50% yield, 84.7% purity based on LCMS) of intermediate 326 as a yellow oil.

### Preparation of Intermediate 327

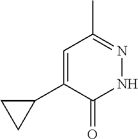

To a solution of intermediate 326 (2.40 g, 8.61 mmol, 84.7% purity based on LCMS) in dichloromethane (30 mL) was added trifluoroacetic acid (10 mL). The resulting mixture was stirred at 50° C. for 1 hour. The resulting mixture was concentrated under reduced pressure. The residue was dissolved with water and adjusted to PH=7 by ammonium hydroxide (33% in water). The mixture was extracted with dichloromethane for 5 times. The organic layers were combined, washed with brine, dried over sodium sulfate, filtered and concentrated under reduced pressure to give 870 mg (58% yield) of intermediate 327 as a yellow solid.

### Preparation of Intermediate 328

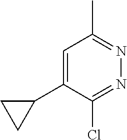

To a solution of intermediate 327 (2.46 g, 16.38 mmol) in acetonitrile (50 mL) was added POCl₃ (7.6 mL, 81.90 mmol). After stirring at 50° C. overnight, the reaction solution was poured into ice water slowly. The resulting solution was adjusted to pH=7 by a solution of NaOH (2 M in water) and extracted with ethyl acetate. The combined organic layers were washed with water and brine and dried over Na₂SO₄. The solid was filtered off. The filtrate was concentrated under reduced pressure. The residue was purified by flash silica gel column chromatography (EA/PE, 0% EA to 30% EA) to give 2.1 g of intermediate 328 as a yellow oil.

### Preparation of Intermediate 329

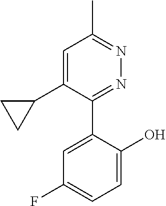 [SMILES: Cc1cc(C2CC2)c(-c2cc(F)ccc2O)nn1]

To a mixture of intermediate 328 (5.0 g, 29.65 mmol), 4-fluoro-2-(4,4,5,5-tetramethyl-1,3,2-dioxaborolan-2-yl)phenol (7.06 g, 29.65 mmol) and Pd(PPh₃)₄ (1.71 g, 1.48 mmol) in 1,4-dioxane (75 mL) was added sodium carbonate solution (25 mL, 2 M in water, 50.00 mmol) was stirred 90° C. for 3 hours. After cooling to room temperature, the reaction solution was quenched with water and extracted with ethyl acetate. The combined organic layers were washed with water and brine and dried over Na₂SO₄. The filtrate was concentrated under reduced pressure. The residue was purified by flash silica gel column chromatography (EA/PE, 0% EA to 60% EA) to give 6.0 g of intermediate 329 as a yellow solid.

### Preparation of Intermediate 330

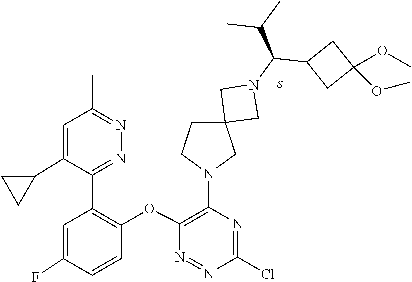

To a solution of intermediate 307 (8 g, 18.6 mmol) in tetrahydrofuran (200 mL) were added intermediate 329 (5.45 g, 22.31 mmol) and 1,8-diazabicyclo[5.4.0]undec-7-ene (6.94 mL, 46.47 mmol). The resulting mixture was stirred at room temperature over weekend. The reaction was quenched with water and extracted with ethyl acetate. The organic layers were combined, dried over sodium sulfate, filtered and concentrated under reduced pressure to give a residue. The residue was purified by flash silica gel column chromatography (EA/PE, 0% EA to 90% EA) to afford 7.56 g (62% yield) of intermediate 330 as a yellow solid.

### Preparation of Intermediate 331

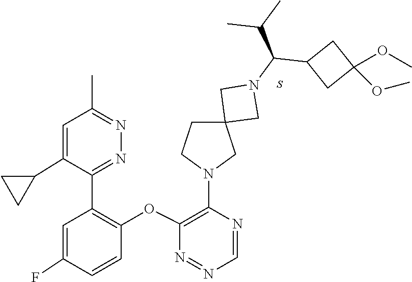

To a solution of intermediate 330 (7.26 g, 10.92 mmol) in THE (140 mL) were added Pd(dppf)Cl₂ (446 mg, 0.55 mmol), NaBH₄ (702 mg, 18.57 mmol) and TMEDA (2.78 mL, 18.57 mmol). After stirring at room temperature under nitrogen atmosphere overnight, the reaction was quenched with water and extracted with ethyl acetate. The organic layers were combined, dried over sodium sulfate, filtered and concentrated under reduced pressure. The residue was purified by flash silica gel column chromatography (EA/PE, 0% EA to 91% EA) to afford 416 mg (65% yield) of intermediate 331 as a yellow solid.

### Preparation of Intermediate 332

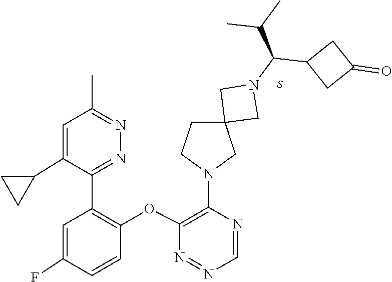

To a solution of intermediate 331 (500 mg, 0.75 mmol) in acetone (7.5 mL) and water (2.5 mL) was added TsOH (649 mg, 3.77 mmol). After stirring at 65° C. overnight, the reaction was quenched with water and extracted with ethyl acetate. The organic layers were combined, dried over sodium sulfate, filtered and concentrated under reduced pressure to give 480 mg (98% yield) of intermediate 332 as a brown solid.

### Preparation of Intermediate 333

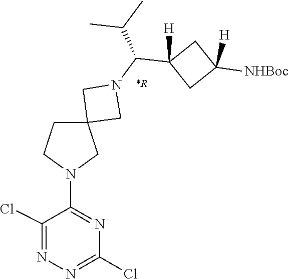 [SMILES: CC(C)[C@H]([C@H]1C[C@@H](NC(=O)OC(C)(C)C)C1)N1CC2(CCN(c3nc(Cl)nnc3Cl)C2)C1]

A stir bar, intermediate 283 (2.7 g, 6.58 mmol), 3,5,6-trichloro-1,2,4-triazine (1.21 g, 6.56 mmol) in DCM (100 mL) was stirred at 25° C. for 10 min and added TEA (2.74 mL, 19.7 mmol). The mixture was stirred at 25° C. for 10 h. The mixture was poured into water (100 mL×2) and extracted with dichloromethane (50 mL×2). The combined organic extracts were washed with brine (50 mL), dried over anhydrous Na₂SO₄, filtered and concentrated under reduced pressure to afford the crude product which was purified by FCC (eluent: dichloromethane:methanol=1:0 to 10:1) to give intermediate 333 as a yellow solids (3.32 g, 81.7% yield).

### Preparation of Intermediate 336

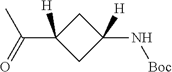

A solution of intermediate 5 (9.91 g, 38.364 mmol) in anhydrous THF (191.8 mL) was cooled to 0° C. 3.4 M MeMgBr in THF (25.952 mL, 3.4 M, 88.236 mmol) was slowly added. Once the addition was complete, the reaction was allowed to warm to rt and stirred over the weekend. The reaction was quenched by addition of sat. ammonium chloride solution. The water phase was extracted several times with diethyl ether. The organic extracts were combined, dried over magnesium sulfate, filtered and concentrated to afford the crude material (8 g, yield 97.775%). The material was purified by FCC (silica gel, 10% to 30% EA in n-heptane) to yield Compound 336 (1.16 g, 14.2% yield) as a white powder.

### Preparation of Intermediate 338

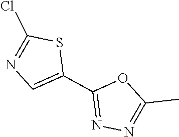

2-chloro-1,3-thiazole-5-carboxylic acid (0.5 g, 3.057 mmol) was dissolved in EtOAc (5.2 mL) and treated with T3P 50% in EtOAc (4.41 mL) and acetohydrazide (226 mg, 3.057 mmol). The resulting solution was stirred over the weekend at 70° C. The reaction mixture was hydrolysed and extracted with ethyl acetate (3×). The combined organic phases were washed with brine. The volatile components were removed at rotavap. The material was analyzed with HPLC and NMR, showing intermediate 338 (240 mg, 39% yield) with T3P impurities. The material was used without further purification.

### Preparation of Intermediate 339

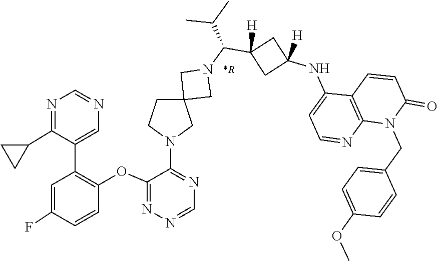 [SMILES: COc1ccc(Cn2c(=O)ccc3c(N[C@H]4C[C@@H]([C@@H](C(C)C)N5CC6(CCN(c7ncnnc7Oc7ccc(F)cc7-c7cncnc7C7CC7)C6)C5)C4)ccnc32)cc1]

tBuXPhos Pd G3 (36.5 mg, 0.046 mmol) was added to a solution of Compound 1a (250 mg, 0.459 mmol), 5-chloro-1-(4-methoxybenzyl)-1,8-naphthyridin-2(1H)-one (262 mg, 0.871 mmol) and NaOtBu (132 mg, 1.37 mmol) in 1,4-dioxane (8 mL) under argon atmosphere. The mixture was stirred at 100° C. under microwave for 1 h. The mixture was cooled to room temperature, diluted with dichloromethane (20 mL), washed with H₂O (10 mL) and brine (20 mL). The organic layers were dried over anhydrous Na₂SO₄, filtered and concentrated under reduced pressure to give a residue, which was purified by flash column chromatography on silica gel (eluent: dichloromethane:methanol=1:0 to 10:1) to give intermediate 339 (180 mg, 37.06% yield) as a yellow oil.

### Preparation of Intermediate 340 (1-(Benzyloxy)-4-Fluoro-2-Nitrobenzene

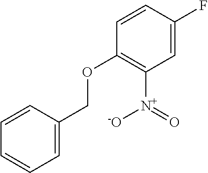

To a solution of 4-fluoro-2-nitro (5 g, 31.827 mmol) and Cs₂CO₃ (20.74 g, 63.653 mmol) in DMF (50 mL) was added benzyl bromide (4 mL, 33.418 mmol) at room temperature for 6 h. Upon completion (TLC), reaction mixture was diluted with EtOAc (100 mL) and washed with water (200 mL). Layers were separated and the aqueous layer was extracted with EtOAc (3×50 mL). Combined organic layer was washed with water, brine, dried over anhydrous MgSO₄, filtered, and concentrated in vacuo. The residue was purified by flash column chromatography (0 to 10% EtOAc in heptane) to afford Intermediate 340 (1-(benzyloxy)-4-fluoro-2-nitrobenzene) (7.85 g, yield 99%) a thick yellow oil.

### Preparation of Intermediate 341 (2-benzyloxy-5-fluoro-aniline

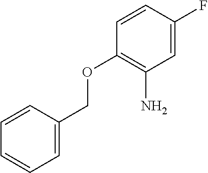 [SMILES: Nc1cc(F)ccc1OCc1ccccc1]

To a mixture of Intermediate 340 (1 g, 4.045 mmol) and NH₄Cl (2.15 g, 40.196 mmol) in EtOH (30 mL) at ambient temperature was added Zinc powder (2.63 g, 40.208 mmol) and the mixture was then heated to 50° C. overnight. The mixture was diluted with EtOAc and filtered on a pad of Celite® and the solvent was removed under reduced pressure. The residue was partitioned between EtOAc (50 mL) and water. Aqueous layer was extracted with EtOAc (2×25 mL). Combined organic layer was washed with water, brine, dried over anhydrous MgSO₄ and rotary evaporated to afford Intermediate 340 (2-benzyloxy-5-fluoro-aniline) (875 mg, yield 99%) as a brown oil.

### Preparation of Intermediate 342 (N-(2-benzyloxy-5-fluoro-phenyl)cyclopropanecarboxamide

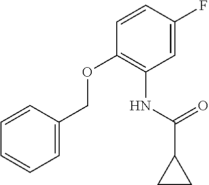 [SMILES: O=C(Nc1cc(F)ccc1OCc1ccccc1)C1CC1]

To a solution of Intermediate 341 (2-benzyloxy-5-fluoro-aniline) (1.37 g, 6.306 mmol) and Et₃N (2.64 mL, 19.92 mmol) in anhydrous dichloromethane (20 mL) at 0° cyclopropane carbonyl chloride (0.7 mL, 7.57 mmol) was added, and the reaction mixture was stirred at room temperature for 1 h.

Upon completion (TLC), reaction mixture was diluted with dichloromethane (100 mL) and washed with water (100 mL). Aqueous layer was extracted with dichloromethane (30 mL×2), combined organic layer was washed with brine, dried over anhydrous MgSO₄, filtered, and concentrated in vacuo. The residue was purified by flash column chromatography (0 to 60% EtOAc in heptane) to afford Intermediate 342 (N-(2-benzyloxy-5-fluoro-phenyl)cyclopropanecarboxamide) (1.55 g, yield 86%) as a colourless solid.

### Preparation of Intermediate 343 (N-(2-benzyloxy-5-fluoro-phenyl)cyclopropanecarbothioamide

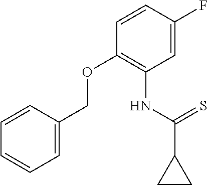 [SMILES: Fc1ccc(OCc2ccccc2)c(NC(=S)C2CC2)c1]

To a solution of Intermediate 342 (N-(2-benzyloxy-5-fluoro-phenyl)cyclopropanecarboxamide) (1 g, 3.505 mmol) in 1,4-dioxane (30 mL), Lawesson's reagent (0.8 g, 1.963 mmol) was added and the resulting mixture was heated to 100° C. for 6 h. Reaction mixture was then concentrated in vacuo and the residue was purified by flash column chromatography (0 to 15% EtOAc in heptane) to afford Intermediate 343 (N-(2-benzyloxy-5-fluoro-phenyl)cyclopropanecarbothioamide) (0.815 g, 77%) as a pale yellow solid

### Preparation of Intermediate 344 (4-(2-benzyloxy-5-fluoro-phenyl)-3-cyclopropyl-5-methyl-1,2,4-triazole

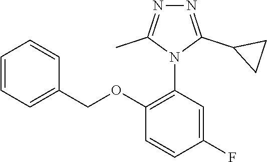 [SMILES: Cc1nnc(C2CC2)n1-c1cc(F)ccc1OCc1ccccc1]

Hydrazine hydrate (0.28 mL, 2.883 mmol) was added dropwise to a stirred solution of Intermediate 343 (N-(2-benzyloxy-5-fluoro-phenyl)cyclopropanecarbothioamide) (790 mg, 2.622 mmol) in THE (20 mL) at ambient temperature. After 60 min, the solution was concentrated under reduced pressure and the residue was treated with triethyl orthoacetate (5 mL). The mixture was heated at 80° C. for 30 min, cooled to ambient temperature, and concentrated under reduced pressure. The residue was treated with ice-cold dilute aqueous ammonia (15 mL), water (25 mL) and extracted with EtOAc (70 mL×3). Combined organic layer was washed with brine, dried over anhydrous MgSO₄ and rotary evaporated. The residue was purified by flash column chromatography (0 to 100% EtOAc in heptane) to afford Intermediate 344 (4-(2-benzyloxy-5-fluoro-phenyl)-3-cyclopropyl-5-methyl-1,2,4-triazole) (530 mg, yield 62%) as a cream coloured fluffy solid.

### Preparation of Intermediate 345 (2-(3-cyclopropyl-5-methyl-1,2,4-triazol-4-yl)-4-fluoro-phenol

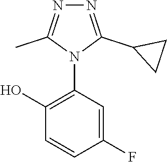 [SMILES: Cc1nnc(C2CC2)n1-c1cc(F)ccc1O]

Pd/C (10%) (27 mg) was added to a solution of Intermediate 344 (4-(2-benzyloxy-5-fluoro-phenyl)-3-cyclopropyl-5-methyl-1,2,4-triazole) (200 mg, 0.62 mmol) in methanol (50 mL) and kept for hydrogenation at ambient temperature overnight. Upon completion (TLC), catalyst was filtered off through a Celite® bed, washed with MeOH several times and combined organic layer was concentrated in vacuo to give Intermediate 345 (2-(3-cyclopropyl-5-methyl-1,2,4-triazol-4-yl)-4-fluoro-phenol) (135 mg, yield 93%) as a colourless solid.

### Preparation of Intermediate 346 (7-[3-chloro-6-[2-(3-cyclopropyl-5-methyl-1,2,4-triazol-4-yl)-4-fluoro-phenoxy]-1,2,4-triazin-5-yl]-2-[(1R)-1-(3,3-dimethoxycyclobutyl)-2-methyl-propyl]-2,7-diazaspiro[3.4]octane

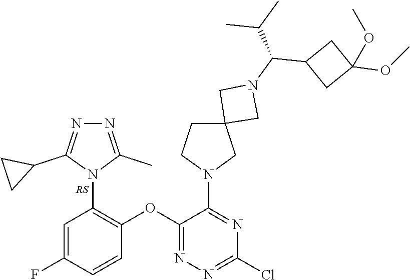 [SMILES: COC1(OC)CC([C@@H](C(C)C)N2CC3(CCN(c4nc(Cl)nnc4Oc4ccc(F)cc4-n4c(C)nnc4C4CC4)C3)C2)C1]

A mixture of intermediate 307 (130 mg, 0.3 mmol), Intermediate 345 (2-(3-cyclopropyl-5-methyl-1,2,4-triazol-4-yl)-4-fluoro-phenol) (70 mg, 0.3 mmol) and DBU (0.225 mL, 1.5 mmol) in THE (4 mL) was stirred at RT for 48 h. Upon completion, reaction mixture was diluted with EtOAc (30 mL) and washed with water. Layers were separated and aqueous layer was extracted with EtOAc (2×50 mL). Combined organic layer was washed with water, brine, dried over anhydrous MgSO₄ and rotary evaporated. The crude compound was purified by flash column chromatography (0 to 70% n-heptanes in EtOAc) to afford Intermediate 346 (60 mg, yield 31%) as a colourless oil.

### Preparation of Intermediate 347 (7-[6-[2-(3-cyclopropyl-5-methyl-1,2,4-triazol-4-yl)-4-fluoro-phenoxy]-1,2,4-triazin-5-yl]-2-[(1R)-1-(3,3-dimethoxycyclobutyl)-2-methyl-propyl]-2,7-diazaspiro[3.4]octane

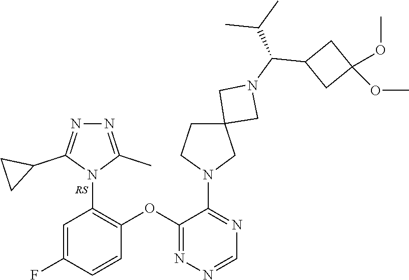 [SMILES: COC1(OC)CC([C@@H](C(C)C)N2CC3(CCN(c4ncnnc4Oc4ccc(F)cc4-n4c(C)nnc4C4CC4)C3)C2)C1]

A solution of Intermediate 346 (60 mg, 0.0957 mmol) and N,N,N′,N′-tetramethylethylenediamine (50 μL, 0.335 mmol) in THE (10 mL) was degassed for 5 min then Pd(dppf)Cl₂.DCM (12 mg, 0.0144 mmol) and NaBH₄ (26 mg, 0.67 mmol) were added and the mixture was purged with nitrogen (3 times) and stirred at room temperature for 18 h. The reaction mixture was quenched with a 10% aqueous solution of K₂CO₃, then extracted with dichloromethane (3×25 mL). Combined organic layer was washed with brine, dried over anhydrous MgSO₄, filtered, evaporated in vacuo and purified by flash column chromatography (0 to 3% MeOH in dichloromethane as eluents) to afford Intermediate 347 (30 mg, yield 52%) as a colourless fluffy solid as mixture of atropisomers.

### Preparation of Intermediate 348 (3-[(1R)-1-[7-[6-[2-(3-cyclopropyl-5-methyl-1,2,4-triazol-4-yl)-4-fluoro-phenoxy]-1,2,4-triazin-5-yl]-2,7-diazaspiro[3.4]octan-2-yl]-2-methyl-propyl]cyclobutanone

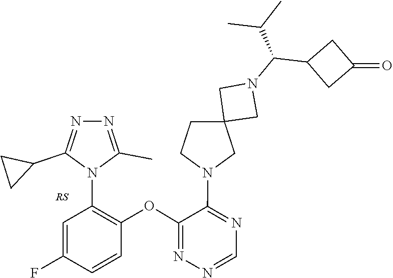 [SMILES: Cc1nnc(C2CC2)n1-c1cc(F)ccc1Oc1nncnc1N1CCC2(C1)CN([C@H](C(C)C)C1CC(=O)C1)C2]

To a solution of Intermediate 347 (252 mg, 0.425 mmol) in anhydrous dichloromethane (3 mL) was added TFA (0.65 mL, 8.5 mmol) and the mixture was stirred at ambient for 2 h. The reaction mixture then diluted with dichloromethane. The organic layer was washed with sat. NaHCO₃, then dried over MgSO₄, filtered and evaporated in vacuo to give Intermediate 348 (233 mg) as a cream coloured fluffy solid as a mixture of atropisomers.

### Preparation of Intermediate 349 (5-cyclopropyl-3-methyl-isoxazole

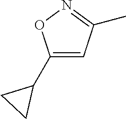

A mixture of 1-cyclopropylbutane-1,3-dione (5 g, 39.634 mmol), NH₂OH·HCl (3.31 g, 47.56 mmol) in EtOH was heated under microwave irradiation at 130° C. for 5 min. The mixture was added water (25 mL) and then extracted with EtOAc (3×). The organic layer was separated, dried over anhydrous MgSO₄, and concentrated under vacuum. The residue was subjected to flash column purification (0 to 90% EtOAc in heptane as eluents) to yield 5-cyclopropyl-3-methyl-isoxazole as a major regio-isomer (2.4 g, 44%). The mixture of regio-isomers was used in the next step without separation.

### Preparation of Intermediate 350 (4-bromo-5-cyclopropyl-3-methyl-isoxazole

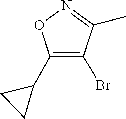

N-bromosuccinimide (1.532 g, 8.607 mmol) was added to a solution of Intermediate 349 (0.5 g, 4.06 mmol) in DMF (8 mL) and stirred at room temperature for 4 h Upon completion (LCMS), water was added to the reaction mixture extracted with diethyl ether (3×25 mL). Combined organic layer was washed with brine, dried over MgSO₄, filtered, and concentrated under vacuum. The residue was subjected to flash column purification (0 to 90% EtOAc in heptane as eluents) to yield Intermediate 350 as a major regio-isomer 600 mg, 73%).

### Preparation of Intermediate 351 (5-cyclopropyl-3-methyl-4-(4,4,5,5-tetramethyl-1,3,2-dioxaborolan-2-yl)isoxazole

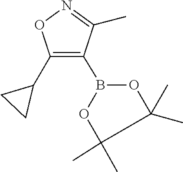 [SMILES: Cc1noc(C2CC2)c1B1OC(C)(C)C(C)(C)O1]

Intermediate 350 (6 g, 29.696 mmol) was dissolved in THE (250 mL) and cooled to −78° C. under N₂. n-BuLi (2.5 M in hexane) (17.8 mL, 2.5 M, 44.5 mmol) was added slowly to the solution, which was then stirred for 30 min at −78° C. 2-Isopropoxy-4,4,5,5-tetramethyl-1,3,2-dioxaborolane (7.27 mL, 35.635 mmol) was added to the reaction mixture and stirred at −78° C. for an additional 2 h and then it was warmed to rt and stirred overnight. The reaction was quenched by adding NH₄Cl saturated solution. The mixture was then extracted with EtOAc, washed with brine, dried over anhydrous MgSO₄, filtered, and concentrated in vacuo. The residue was purified by flash column chromatography (0 to 5% EtOAc in heptane as eluents) to afford Intermediate 51 (5 g, 67%) as a regio-isomeric mixture.

### Preparation of Intermediate 352 (5-cyclopropyl-4-(5-fluoro-2-methoxy-phenyl)-3-methyl-isoxazole

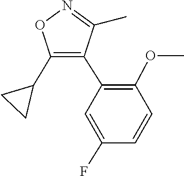 [SMILES: COc1ccc(F)cc1-c1c(C)noc1C1CC1]

To a solution of Intermediate 351 (1.823 g, 7.316 mmol) and 2-bromo-4-fluoro-1-methoxy-benzene (1 g, 4.877 mmol) in dioxane (250 mL), saturated aq. NaHCO₃ (50 mL) was added and the mixture was degassed for 10 min. To it, [Ph₃P]₄Pd (1.127 g, 0.975 mmol) was added and the reaction mixture was stirred at 60° C. for 2 h. Dichloromethane and water were added to the mixture and layers were separated. Aqueous layer was washed with dichloromethane (2 x). Combined organic layer was washed with brine, dried over MgSO₄, filtered, and evaporated in vacuo. The residue was purified by flash column chromatography (0 to 70% EtOAc in heptane as eluents) to give 5-cyclopropyl-4-(5-fluoro-2-methoxy-phenyl)-3-methyl-isoxazole (0.65 g, 53%).

### Preparation of Intermediate 353 (2-(5-cyclopropyl-3-methyl-isoxazol-4-yl)-4-fluoro-phenol

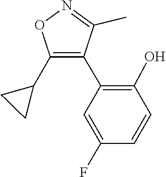 [SMILES: Cc1noc(C2CC2)c1-c1cc(F)ccc1O]

A solution of Intermediate 352 (150 mg, 0.607 mmol) in dichloromethane (10 mL) was cooled to a temperature between 5-10° C. To it, boron tribromide (169 μL, 1.82 mmol) was added dropwise. The resulting reaction mixture was then stirred at 0° C. for 2.5 h. Water (10 mL) was added to the mixture and layers were separated. Aqueous layer was extracted with dichloromethane (2 x). Combine organic layer was washed with brine, dried over anhydrous MgSO₄, filtered, and evaporated in vacuo. The residue was purified by flash column chromatography (0 to 70% EtOAc in heptanes as eluents) to give Intermediate 353 (70 mg, 49%).

### Preparation of Intermediate 354 (4-[2-[[3-chloro-5-[2-[(1R)-1-(3,3-dimethoxycyclobutyl)-2-methyl-propyl]-2,7-diazaspiro[3.4]octan-7-yl]-1,2,4-triazin-6-yl]oxy]-5-fluoro-phenyl]-5-cyclopropyl-3-methyl-isoxazole

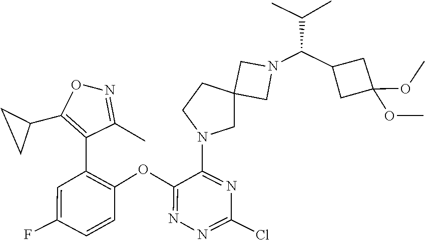 [SMILES: COC1(OC)CC([C@@H](C(C)C)N2CC3(CCN(c4nc(Cl)nnc4Oc4ccc(F)cc4-c4c(C)noc4C4CC4)C3)C2)C1]

A mixture of intermediate 307 (700 mg, 1.626 mmol), Intermediate 353 (392 mg, 1.678 mmol) and DBU (1.2 mL, 8.13 mmol) in THE (90 mL) was stirred at RT for 72 h. Upon completion, reaction mixture was diluted with dichloromethane (100 mL) and washed with water. Layers were separated and aqueous layer was extracted with dichloromethane (2×50 mL). Combined organic layer was washed with water, brine, dried over anhydrous MgSO₄ and rotary evaporated. The residue was purified by flash column chromatography (0 to 2% MeOH in dichloromethane) to afford Intermediate 354 (380 mg, yield 37%).

### Preparation of Intermediate 355 (5-cyclopropyl-4-[5-fluoro-2-[[5-[2-[(1R)-1-(3,3-dimethoxycyclobutyl)-2-methyl-propyl]-2,7-diazaspiro[3.4]octan-7-yl]-1,2,4-triazin-6-yl]oxy]phenyl]-3-methyl-isoxazole

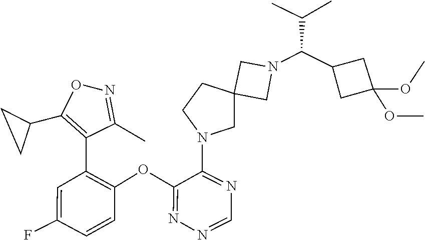 [SMILES: COC1(OC)CC([C@@H](C(C)C)N2CC3(CCN(c4ncnnc4Oc4ccc(F)cc4-c4c(C)noc4C4CC4)C3)C2)C1]

Pd/C (10%) (45 mg) was added to a solution of afford Intermediate 354 (260 mg, 0.415 mmol) and thiophene (0.10 mL, 0.4 M, 0.041 mmol) in MeOH (50 mL) at ambient temperature and the mixture was stirred under H₂ (1 atm) for 1 h. Upon completion (LCMS), the mixture was filtered over dicalite and the solvent was evaporated under vacuum to give Intermediate 355 (100 mg, 41%).

### Preparation of Intermediate 356 (3-[(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutanone

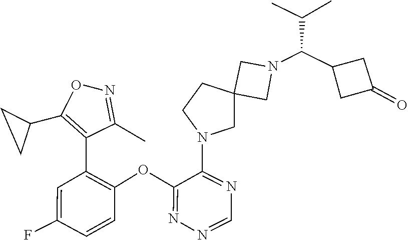 [SMILES: Cc1noc(C2CC2)c1-c1cc(F)ccc1Oc1nncnc1N1CCC2(C1)CN([C@H](C(C)C)C1CC(=O)C1)C2]

To a solution of Intermediate 355 (300 mg, 0.51 mmol) in anhydrous dichloromethane (30 mL) was added trifluoroacetic acid (0.775 mL, 10.123 mmol) and the mixture was stirred at ambient temperature for 2 h. The reaction mixture then diluted with dichloromethane. The organic layer was washed with 10% aqueous solution of Na₂CO₃, then dried over anhydrous MgSO₄, filtered and evaporated in vacuo to afford Intermediate 356 in quantitative yield.

### Preparation of Compounds

### Compound 1

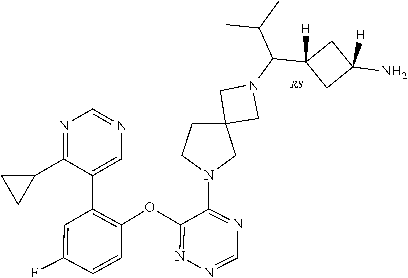 [SMILES: CC(C)C([C@H]1C[C@@H](N)C1)N1CC2(CCN(c3ncnnc3Oc3ccc(F)cc3-c3cncnc3C3CC3)C2)C1]

To a solution of Compound 490 (250 mg, 0.388 mmol) in CH₂Cl₂ (3 mL) was added TFA (2.0 mL, 26 mmol) at 0° C. The mixture was stirred at room temperature for 1 h. The mixture was adjusted to pH=13 with aq. NaOH (2 M). Then, the resultant mixture was extracting with CH₂Cl₂ (10 mL×2). The combined organic extracts were washed with brine (20 mL), dried over Na₂SO₄, filtered, and concentrated to dryness under reduced pressure to yield Compound 1 (185 mg, crude) as a yellow oil, which was used in the next step without additional purification.

The compounds reported below were prepared following an analogous methodology as described for Compound 1 starting from the corresponding intermediates:

| Compound number | Structure |
|---|---|
| Compound 1a | 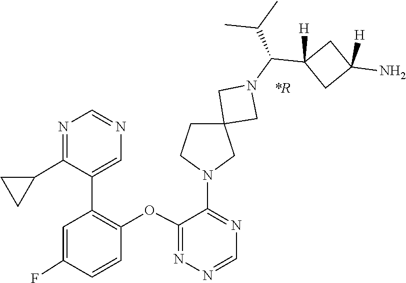 [SMILES: CC(C)[C@H]([C@H]1C[C@@H](N)C1)N1CC2(CCN(c3ncnnc3Oc3ccc(F)cc3-c3cncnc3C3CC3)C2)C1] |
| | from Compound 491a |
| Compound 1b | 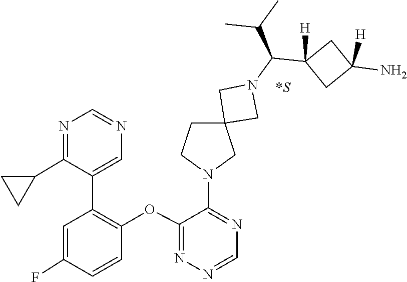 [SMILES: CC(C)[C@@H]([C@H]1C[C@@H](N)C1)N1CC2(CCN(c3ncnnc3Oc3ccc(F)cc3-c3cncnc3C3CC3)C2)C1] |
| | from Compound 491b |
| Compound 359 | 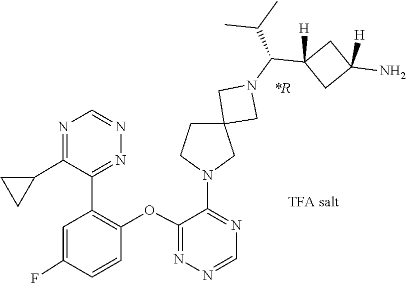 [SMILES: CC(C)[C@H]([C@H]1C[C@@H](N)C1)N1CC2(CCN(c3ncnnc3Oc3ccc(F)cc3-c3nncnc3C3CC3)C2)C1] |
| | from intermediate 284 |
| Compound 360 | 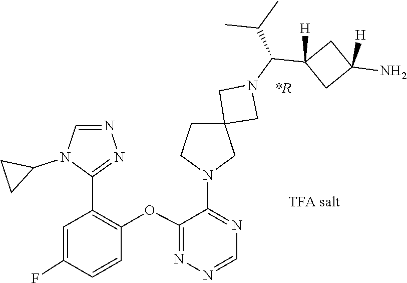 [SMILES: CC(C)[C@H]([C@H]1C[C@@H](N)C1)N1CC2(CCN(c3ncnnc3Oc3ccc(F)cc3-c3nncn3C3CC3)C2)C1] |
| | from intermediate 292 |
| Compound 361 | 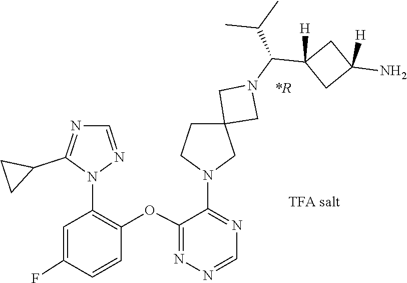 [SMILES: CC(C)[C@H]([C@H]1C[C@@H](N)C1)N1CC2(CCN(c3ncnnc3Oc3ccc(F)cc3-n3ncnc3C3CC3)C2)C1] |
| | from intermediate 276 |
| Compound 362 | 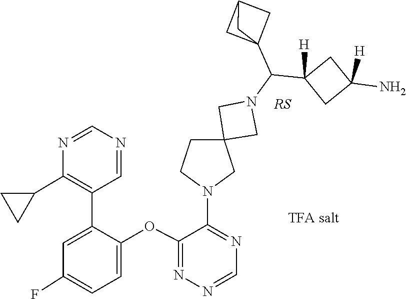 [SMILES: N[C@H]1C[C@@H](C(N2CC3(CCN(c4ncnnc4Oc4ccc(F)cc4-c4cncnc4C4CC4)C3)C2)C23CC(C2)C3)C1] |
| | from intermediate 33 and intermediate 255 |
| Compound 363 | 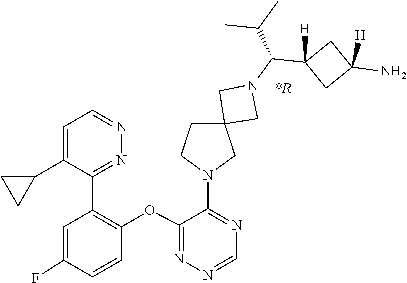 [SMILES: CC(C)[C@H]([C@H]1C[C@@H](N)C1)N1CC2(CCN(c3ncnnc3Oc3ccc(F)cc3-c3nnccc3C3CC3)C2)C1] |
| | from intermediate 297 |
| Compound 364 | 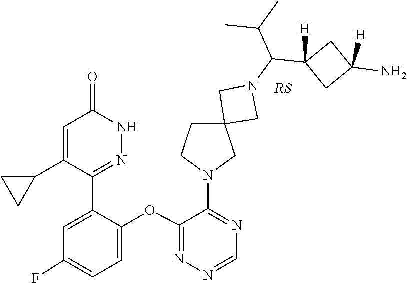 [SMILES: CC(C)C([C@H]1C[C@@H](N)C1)N1CC2(CCN(c3ncnnc3Oc3ccc(F)cc3-c3n[nH]c(=O)cc3C3CC3)C2)C1] |
| | from intermediate 302 |
| Compound 365 | 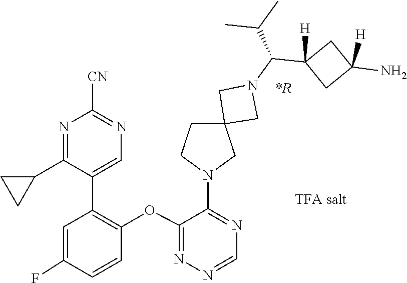 [SMILES: CC(C)[C@H]([C@H]1C[C@@H](N)C1)N1CC2(CCN(c3ncnnc3Oc3ccc(F)cc3-c3cnc(C#N)nc3C3CC3)C2)C1] |
| | from intermediate 73 |
| Compound 366 | 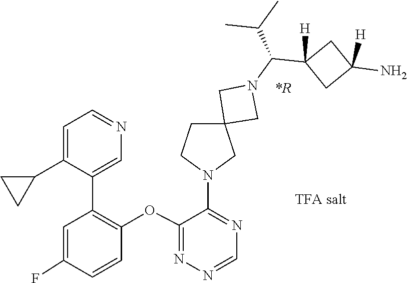 [SMILES: CC(C)[C@H]([C@H]1C[C@@H](N)C1)N1CC2(CCN(c3ncnnc3Oc3ccc(F)cc3-c3cnccc3C3CC3)C2)C1] |
| | from intermediate 77 |
| Compound 369 | 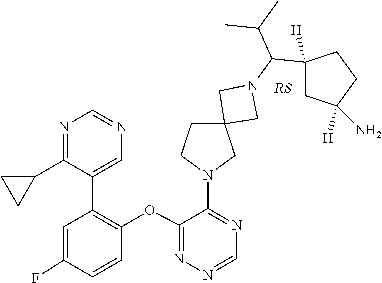 [SMILES: CC(C)C([C@H]1CC[C@@H](N)C1)N1CC2(CCN(c3ncnnc3Oc3ccc(F)cc3-c3cncnc3C3CC3)C2)C1] |
| | from intermediate 33 |

### Alternative Preparation of Compound 1a and Compound 1b

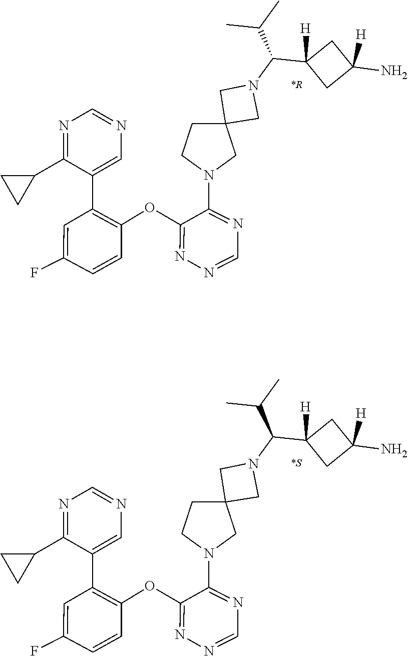 [SMILES: CC(C)[C@H]([C@H]1C[C@@H](N)C1)N1CC2(CCN(c3ncnnc3Oc3ccc(F)cc3-c3cncnc3C3CC3)C2)C1 ; CC(C)[C@@H]([C@H]1C[C@@H](N)C1)N1CC2(CCN(c3ncnnc3Oc3ccc(F)cc3-c3cncnc3C3CC3)C2)C1]

The reaction was performed twice on 6 g of Compound 490. The resulting crude mixtures were combined for the work up and purification.

To a solution of Compound 490 (6 g, 9.4 mmol) in CH₂Cl₂ (150 mL) was added TFA (14 mL, 186 mmol) at 0° C. The mixture was stirred at room temperature for 18 h. The mixture was adjusted to pH=13 with aq. NaOH (2 M). Then, the resultant mixture of both reactions was extracted with CH₂Cl₂ (10 mL×2). The combined organic extracts were washed with brine (20 mL), dried over Na₂SO₄, filtered, and concentrated to dryness under reduced pressure. The residue (10.1 g) was performed via chiral SFC (Stationary phase: Chiralpak IG 5 μm 250*30 mm, Mobile phase: 60% CO2, 40% mixture of EtOH/iPrOH/DCM 40/40/20 v/v/v (+3.0% iPrNH₂)). The pure fractions were collected and the solvent was evaporated under vacuum to give 3.8 g of compound 1a and 3.8 g of compound 1b.

### Preparation of Compound 2

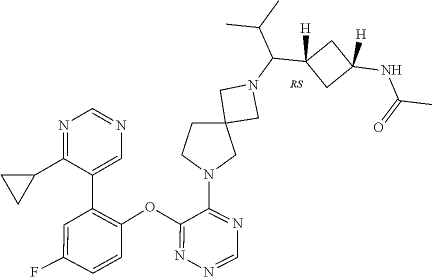 [SMILES: CC(=O)N[C@H]1C[C@@H](C(C(C)C)N2CC3(CCN(c4ncnnc4Oc4ccc(F)cc4-c4cncnc4C4CC4)C3)C2)C1]

To a solution of Compound 1 (150 mg, crude) and acetic acid (36 μL, 0.63 mmol) in CH₂Cl₂ (5 mL) was added T₃P (403 mg, 0.633 mmol, 50% purity) and DIEA (147 μL, 0.828 mmol). The mixture was stirred at 20° C. for 12 h. The mixture was diluted with CH₂Cl₂ (20 mL). The mixture was washed with saturated NaHCO₃ (10 mL), brine (10 mL), dried over Na₂SO₄, filtered and concentrated under reduced pressure to give crude product, which was purified by preparative HPLC (Column: ACE 5 C18-AR 150*30 mm*5 μm, Mobile Phase A: water (10 mM NH₄HCO₃)-ACN, Mobile Phase B: acetonitrile, Flow rate: 30 mL/min, gradient condition from 25% B to 55% B). The pure fractions were collected and the solvent was evaporated under vacuum to give a residue which was partitioned between acetonitrile (2 mL) and water (8 mL). The solution was lyophilized to dryness to yield Compound 2 (60.0 mg) as a white powder.

¹H NMR CDCl₃ (Varian_400 MHz): δ 8.93 (br.s., 1H), 8.46 (s, 1H), 8.40 (br.s., 1H), 7.49-7.31 (m, 1H), 7.26-7.18 (m, 1H), 7.16-7.08 (m, 1H), 6.02 (br.s., 0.2H), 5.60 (br.s., 0.6H), 4.26-4.12 (m, 1H), 3.80-3.42 (m, 4H), 3.19-2.99 (m, 3H), 2.52-2.35 (m, 2H), 2.08-1.98 (m, 3H), 1.94 (s, 3H), 1.89-1.55 (m, 6H), 1.19-0.94 (m, 3H), 0.92-0.62 (m, 7H) ¹⁹F NMR (376 MHz, CDCl₃): −115.85 (s, 1F)

The compound reported below was prepared following an analogous methodology as described for Compound 2 starting from Compound 1:

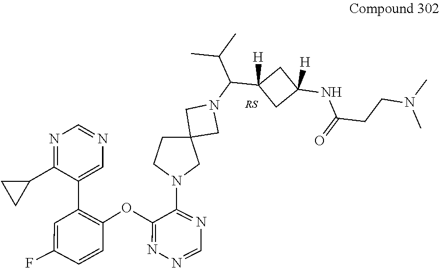 [SMILES: CC(C)C([C@H]1C[C@@H](NC(=O)CCN(C)C)C1)N1CC2(CCN(c3ncnnc3Oc3ccc(F)cc3-c3cncnc3C3CC3)C2)C1]

### Preparation of Compound 2a

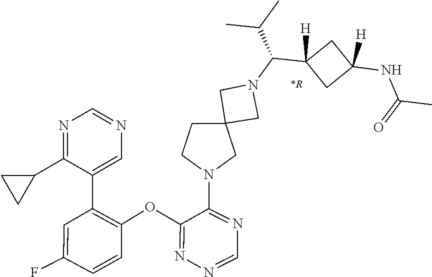 [SMILES: CC(=O)N[C@H]1C[C@@H]([C@@H](C(C)C)N2CC3(CCN(c4ncnnc4Oc4ccc(F)cc4-c4cncnc4C4CC4)C3)C2)C1]

The reaction was performed twice on 1.7 g of compound 1a. The resulting crude mixtures were combined for the work up and purification.

To a solution of compound 1a (1.7 g, 3.12 mmol) and acetic acid (0.4 mL, 7.1 mmol) in DCM (25 mL) was added T₃P (4.3 mL, 7.2 mmol, 50% purity) and DIEA (1.7 mL, 9.4 mmol). The mixture was stirred at 20° C. for 12 h. The mixture was diluted with DCM. The combined mixture of both reactions was washed with saturated NaHCO₃, brine, dried over MgSO₄, filtered and concentrated under reduced pressure. The residue was purified by chromatography over silica gel (mobile phase: gradient from 0.1% NH₄OH, 5% MeOH, 95% DCM to 0.1% NH₄OH, 8% MeOH, 92% DCM). The pure fractions were collected and the solvent was evaporated under vacuum to give a residue which was partitioned between acetonitrile (2 mL) and water (8 mL). The solution was lyophilized to yield compound 2a (1.69; 46%) as a white powder.

¹H NMR (500 MHz, DMSO-d₆) δ ppm 8.80-9.02 (m, 1H), 8.41 (br s, 2H), 7.98 (br d, J=7.2 Hz, 1H), 7.52-7.63 (m, 1H), 7.39-7.50 (m, 2H), 3.95 (dq, J=16.2, 8.2 Hz, 1H), 3.37-3.83 (m, 4H), 3.05 (br s, 3H), 2.84-2.99 (m, 1H), 2.13-2.31 (m, 2H), 1.89-2.03 (m, 3H), 1.78-1.88 (m, 1H), 1.65-1.77 (m, 5H), 1.50-1.64 (m, 2H), 0.61-1.18 (m, 10H)

### Alternative Preparation of Compound 2a and Compound 2b

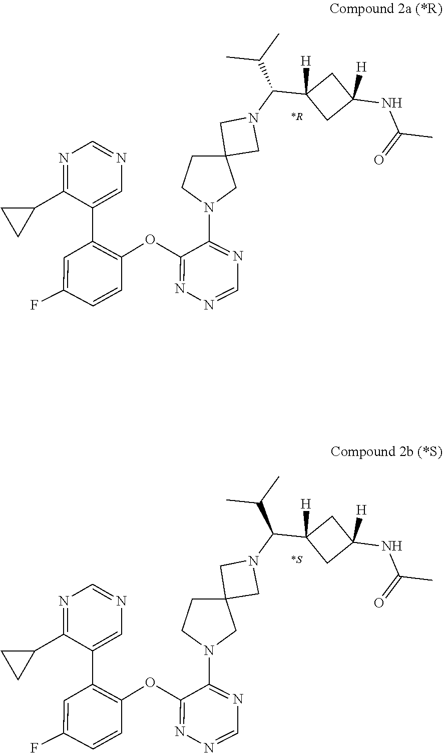 [SMILES: CC(=O)N[C@H]1C[C@@H]([C@@H](C(C)C)N2CC3(CCN(c4ncnnc4Oc4ccc(F)cc4-c4cncnc4C4CC4)C3)C2)C1 ; CC(=O)N[C@H]1C[C@@H]([C@H](C(C)C)N2CC3(CCN(c4ncnnc4Oc4ccc(F)cc4-c4cncnc4C4CC4)C3)C2)C1]

Compound 2 (30 mg, 0.051 mmol) was separated by SFC (column: DAICEL CHIRALCEL OD-H (250 mm*30 mm, 5 μm), eluent: 30% (v/v) super critical CO₂ in 0.1% NH₃H₂O EtOH, flow rate: 50 mL/min). The desired fractions were collected and the solvent was evaporated in vacuo. The residue was redissolved in ACN and water and lyophilized to afford Compound 2a (13 mg, 43% yield) and Compound 2b (11 mg, 37% yield) both as a white powder.

### Preparation of Compound 303

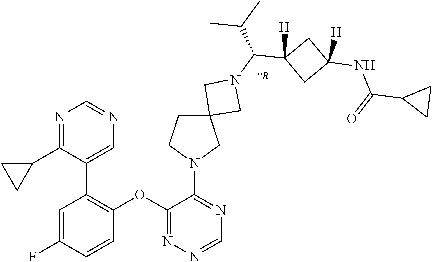 [SMILES: CC(C)[C@H]([C@H]1C[C@@H](NC(=O)C2CC2)C1)N1CC2(CCN(c3ncnnc3Oc3ccc(F)cc3-c3cncnc3C3CC3)C2)C1]

To a solution of Compound 1a (100 mg, 0.184 mmol) and cyclopropanecarboxylic acid (36.2 mg, 0.420 mmol) in DCM (5 mL) were added T3P (268 mg, 0.421 mmol, 50% in EtOAc) and DIEA (118 mg, 0.913 mmol). The resulting mixture was stirred at 25° C. for 12 h. The mixture was diluted with DCM (10 mL) and washed with H₂O (10 mL) and brine (10 mL). After dried over Na₂SO₄, the mixture was filtered and the filtrate was concentrated under reduced pressure to give the crude product, which was purified by preparative HPLC (Column: Phenomenex Gemini-NX 150*30 mm*5 μm, Mobile Phase A: water (0.04% NH₃H₂O+10 mM NH₄HCO₃), Mobile Phase B: ACN, Flow rate: 30 mL/min, gradient condition from 40% B to 70%). The desired fractions were collected and lyophilized to afford Compound 303 (50 mg, 44% yield) as a white powder.

1H NMR CDCl₃ (Bruker_400 MHz): δ 8.94 (s, 1H), 8.46 (s, 1H), 8.44-8.34 (m, 1H), 7.39 (s, 1H), 7.26-7.18 (m, 1H), 7.17-7.08 (m, 1H), 6.31-5.68 (m, 1H), 4.29-4.13 (m, 1H), 3.82-3.30 (m, 4H), 3.26-2.91 (m, 4H), 2.56-2.35 (m, 2H), 2.20-1.93 (m, 4H), 1.80-1.55 (m, 4H), 1.36-1.22 (m, 1H), 1.20-1.03 (m, 2H), 1.02-0.91 (m, 3H), 0.91-0.79 (m, 7H), 0.76-0.64 (in, 2H).

The compounds reported below were prepared following an analogous methodology as described for Compound 2a or Compound 303, starting from the appropriate starting materials (e.g. Compound 1a or other appropriate starting materials):

| Compound number | Structure |
|---|---|
| Compound 356 | 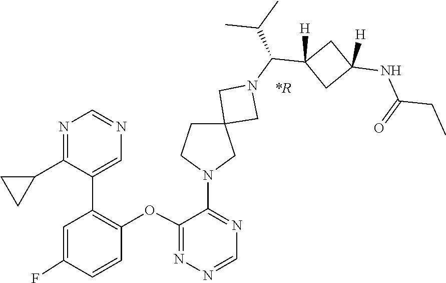 [SMILES: CCC(=O)N[C@H]1C[C@@H]([C@@H](C(C)C)N2CC3(CCN(c4ncnnc4Oc4ccc(F)cc4-c4cncnc4C4CC4)C3)C2)C1] |
| Compound 296 | 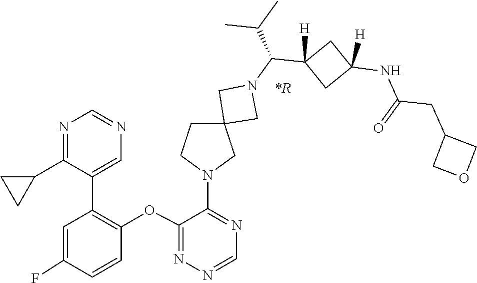 [SMILES: CC(C)[C@H]([C@H]1C[C@@H](NC(=O)CC2COC2)C1)N1CC2(CCN(c3ncnnc3Oc3ccc(F)cc3-c3cncnc3C3CC3)C2)C1] |
| Compound 297 | 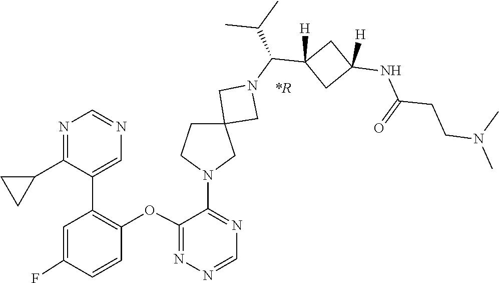 [SMILES: CC(C)[C@H]([C@H]1C[C@@H](NC(=O)CCN(C)C)C1)N1CC2(CCN(c3ncnnc3Oc3ccc(F)cc3-c3cncnc3C3CC3)C2)C1] |
| Compound 298 | 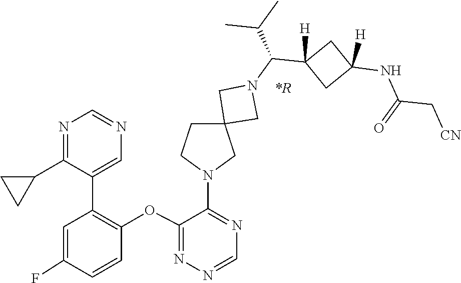 [SMILES: CC(C)[C@H]([C@H]1C[C@@H](NC(=O)CC#N)C1)N1CC2(CCN(c3ncnnc3Oc3ccc(F)cc3-c3cncnc3C3CC3)C2)C1] |
| Compound 299 | 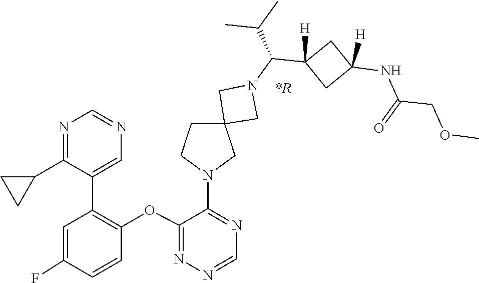 [SMILES: COCC(=O)N[C@H]1C[C@@H]([C@@H](C(C)C)N2CC3(CCN(c4ncnnc4Oc4ccc(F)cc4-c4cncnc4C4CC4)C3)C2)C1] |
| Compound 300 | 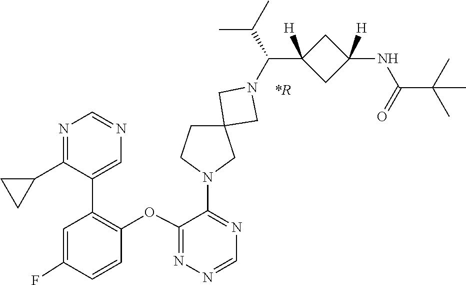 [SMILES: CC(C)[C@H]([C@H]1C[C@@H](NC(=O)C(C)(C)C)C1)N1CC2(CCN(c3ncnnc3Oc3ccc(F)cc3-c3cncnc3C3CC3)C2)C1] |
| Compound 301 | 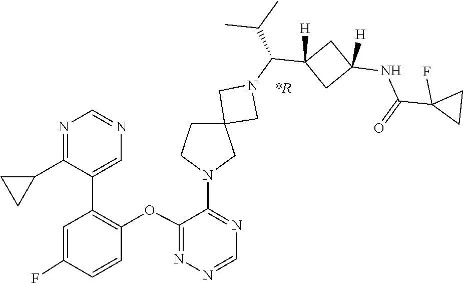 [SMILES: CC(C)[C@H]([C@H]1C[C@@H](NC(=O)C2(F)CC2)C1)N1CC2(CCN(c3ncnnc3Oc3ccc(F)cc3-c3cncnc3C3CC3)C2)C1] |
| Compound 330 | 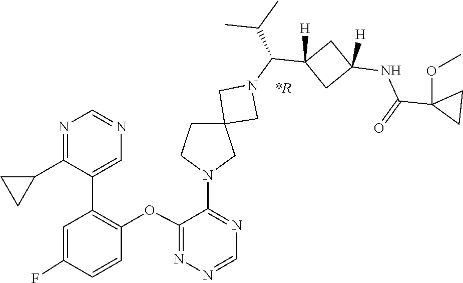 [SMILES: COC1(C(=O)N[C@H]2C[C@@H]([C@@H](C(C)C)N3CC4(CCN(c5ncnnc5Oc5ccc(F)cc5-c5cncnc5C5CC5)C4)C3)C2)CC1] |
| Compound 342 | 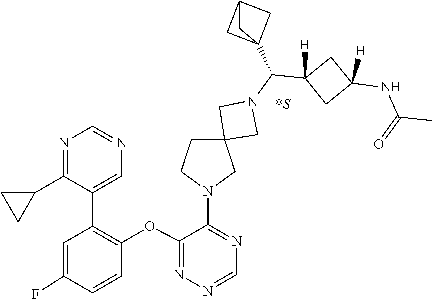 [SMILES: CC(=O)N[C@H]1C[C@@H]([C@H](N2CC3(CCN(c4ncnnc4Oc4ccc(F)cc4-c4cncnc4C4CC4)C3)C2)C23CC(C2)C3)C1] |
| | from Compound 362, SFC separation: first time, REGIS |
| | WHELK-O1 (250 mm*30 mm, 5 um); Mobile phase: A: |
| | Supercritical CO₂, B: 0.1% NH₃H₂O IPA, A:B = 45:55 at 70 |
| | mL/min; second time, RDAICEL CHIRALPAK AD-H |
| | (250 mm*30 mm, 5 um); Mobile phase: A: Supercritical CO₂, |
| | B: 0.1% NH₃H₂O IPA, A:B = 35:65 at 50 mL/min. |
| Compound 344 | 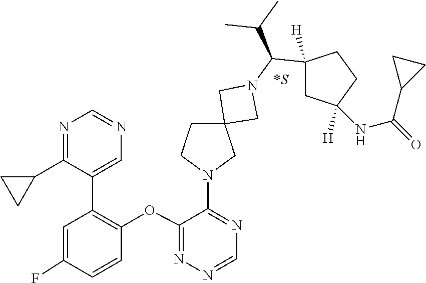 [SMILES: CC(C)[C@@H]([C@H]1CC[C@@H](NC(=O)C2CC2)C1)N1CC2(CCN(c3ncnnc3Oc3ccc(F)cc3-c3cncnc3C3CC3)C2)C1] |
| | from Compound 369, SFC separation: DAICEL |
| | CHIRALPAK IG (250 mm*30 mm, 10 um); Mobile phase: A: |
| | Supercritical CO₂, B: 0.1% NH₃H₂O EtOH, A:B = 65:35 at 50 |
| | mL/min |
| Compound 306 | 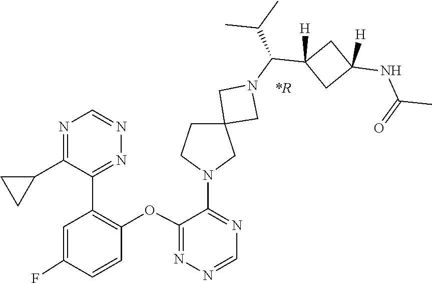 [SMILES: CC(=O)N[C@H]1C[C@@H]([C@@H](C(C)C)N2CC3(CCN(c4ncnnc4Oc4ccc(F)cc4-c4nncnc4C4CC4)C3)C2)C1] |
| | from Compound 359 |
| Compound 307 | 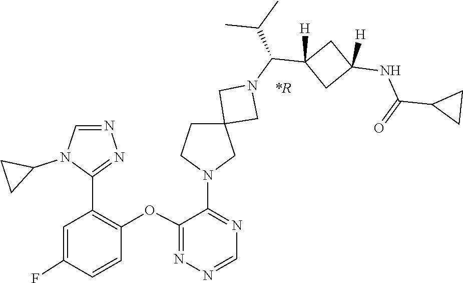 [SMILES: CC(C)[C@H]([C@H]1C[C@@H](NC(=O)C2CC2)C1)N1CC2(CCN(c3ncnnc3Oc3ccc(F)cc3-c3nncn3C3CC3)C2)C1] |
| | from Compound 360 |
| Compound 308 | 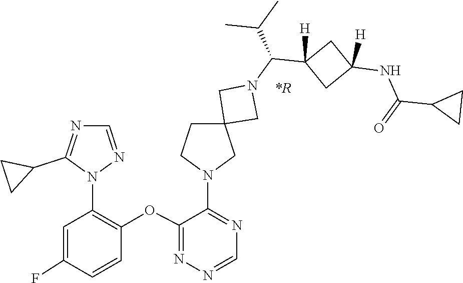 [SMILES: CC(C)[C@H]([C@H]1C[C@@H](NC(=O)C2CC2)C1)N1CC2(CCN(c3ncnnc3Oc3ccc(F)cc3-n3ncnc3C3CC3)C2)C1] |
| | from Compound 361 |
| Compound 309 | 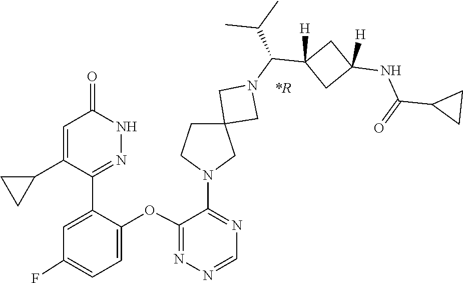 [SMILES: CC(C)[C@H]([C@H]1C[C@@H](NC(=O)C2CC2)C1)N1CC2(CCN(c3ncnnc3Oc3ccc(F)cc3-c3n[nH]c(=O)cc3C3CC3)C2)C1] |
| Compound 310 | 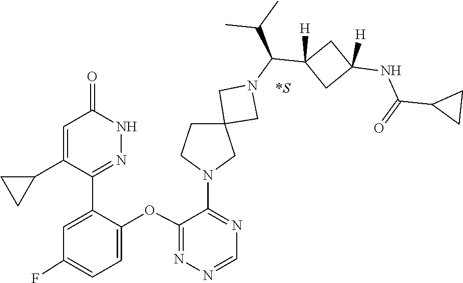 [SMILES: CC(C)[C@@H]([C@H]1C[C@@H](NC(=O)C2CC2)C1)N1CC2(CCN(c3ncnnc3Oc3ccc(F)cc3-c3n[nH]c(=O)cc3C3CC3)C2)C1] |
| | from Compound 364, SFC separation: DAICEL |
| | CHIRALCEL OD-H (250 mm*30 mm, 5 um); Mobile phase: |
| | A: Supercritical CO₂, B: 0.1% NH₃H₂O EtOH, A:B = 70:30 at |
| | 50 mL/min |
| Compound 311 | 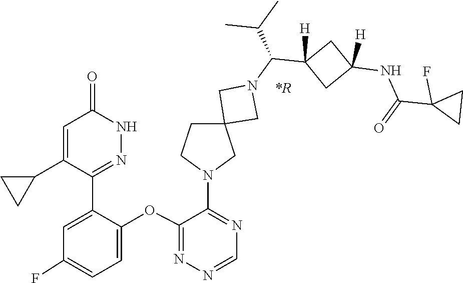 [SMILES: CC(C)[C@H]([C@H]1C[C@@H](NC(=O)C2(F)CC2)C1)N1CC2(CCN(c3ncnnc3Oc3ccc(F)cc3-c3n[nH]c(=O)cc3C3CC3)C2)C1] |
| | from Compound 364, SFC separation: DAICEL |
| | CHIRALPAK IG (250 mm*30 mm, 10 um); Mobile phase: A: |
| | Supercritical CO₂, B: 0.1% NH₃H₂O EtOH, A:B = 65:35 at 50 |
| | mL/min |
| Compound 312 | 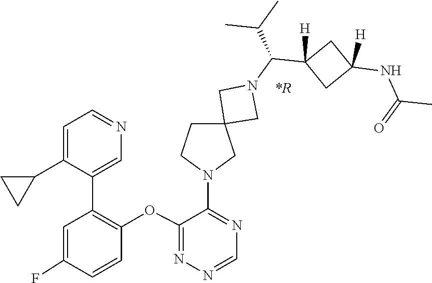 [SMILES: CC(=O)N[C@H]1C[C@@H]([C@@H](C(C)C)N2CC3(CCN(c4ncnnc4Oc4ccc(F)cc4-c4cnccc4C4CC4)C3)C2)C1] |
| | from Compound 366 |
| Compound 343 | 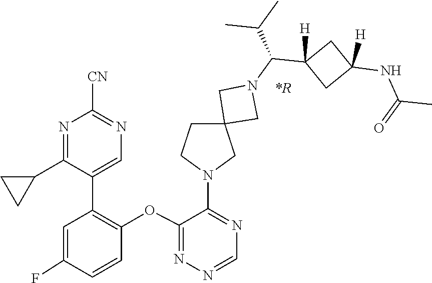 [SMILES: CC(=O)N[C@H]1C[C@@H]([C@@H](C(C)C)N2CC3(CCN(c4ncnnc4Oc4ccc(F)cc4-c4cnc(C#N)nc4C4CC4)C3)C2)C1] |
| | from Compound 365 |
| Compound 27 | 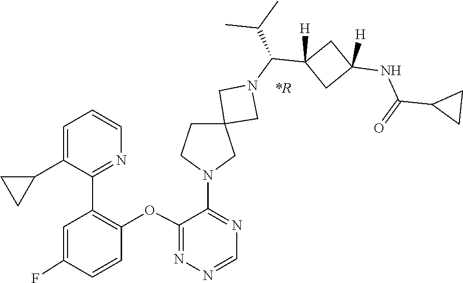 [SMILES: CC(C)[C@H]([C@H]1C[C@@H](NC(=O)C2CC2)C1)N1CC2(CCN(c3ncnnc3Oc3ccc(F)cc3-c3ncccc3C3CC3)C2)C1] |
| | from intermediate 50 and intermediate 6 |

### Preparation of Compound 508

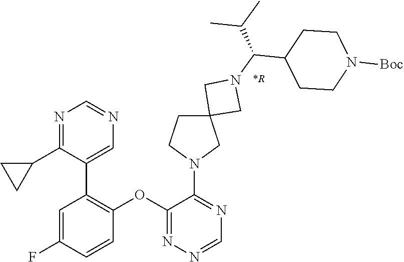 [SMILES: CC(C)[C@H](C1CCN(C(=O)OC(C)(C)C)CC1)N1CC2(CCN(c3ncnnc3Oc3ccc(F)cc3-c3cncnc3C3CC3)C2)C1]

To a solution of intermediate 14 (500 mg, 1.54 mmol) in DMF (0.1 mL) in DCM (30 mL), was added oxalyl dichloride (1.05 g, 8.27 mmol) at 0° C. under N₂ atmosphere. The mixture was stirred at room temperature for 1 hour. Then the mixture was concentrated under reduced pressure (below 35° C.) to give a residue. The residue was dissolved in DCM (30 mL), and TEA (5.0 mL, 35.9 mmol) was added at 0° C. under N₂ atmosphere. The mixture was stirred at 0° C. for 3 minutes. Intermediate 25 (600 mg, 1.55 mmol) in DCM (2 mL) was added dropwise at 0° C. under N₂ atmosphere. The mixture was stirred at r.t. for 1 hour. The reaction mixture was diluted with DCM (50 mL), washed with H₂O (20 mL) and brine (20 mL). The organic layer was dried over anhydrous Na₂SO₄, filtered and concentrated under reduced pressure to give a residue. The residue was purified by FCC (PE:EtOAc=1:3 to 0:1) to yield Compound 508 (260 mg, 18% yield) as a white solid.

### Preparation of Compound 3

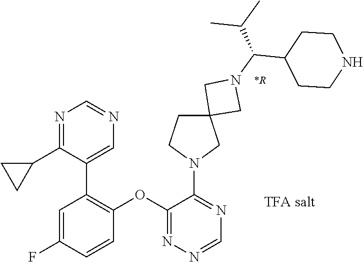 [SMILES: CC(C)[C@H](C1CCNCC1)N1CC2(CCN(c3ncnnc3Oc3ccc(F)cc3-c3cncnc3C3CC3)C2)C1]

To a solution of Compound 508 (260 mg, 0.395 mmol) in dioxane (5 mL) was added 4 M HCl/dioxane (3.00 mL, 12 mmol). The mixture was stirred at room temperature for 0.5 hours. The reaction mixture was concentrated to give Compound 3 (240 mg, crude HCl salt) as a light brown solid (no further purification).

### Preparation of Compound 4

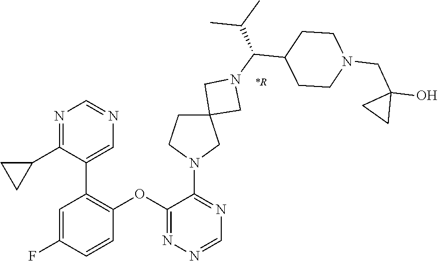 [SMILES: CC(C)[C@H](C1CCN(CC2(O)CC2)CC1)N1CC2(CCN(c3ncnnc3Oc3ccc(F)cc3-c3cncnc3C3CC3)C2)C1]

To a solution of intermediate 29 (370 mg, crude) in DCM (3 mL) was added 4 M HCl/dioxane (0.2 mL, 0.8 mmol). The reaction mixture was stirred at r.t. for 0.5 hours. The reaction mixture was concentrated. The resulting residue was first purified by prep-HPLC (Column: Waters Xbridge Prep OBD C18 150*40 mm*10 um, Mobile Phase A: water (10 mM NH₄HCO₃), Mobile Phase B: acetonitrile, Flow rate: 25 mL/min, gradient condition from 30% B to 80% B), and then by prep-HPLC (Column: Boston Prime C18 150*30 mm*5 um, Mobile Phase A: water (0.04% NH₃H₂O+10 mM NH₄HCO₃), Mobile Phase B: acetonitrile, Flow rate: 25 mL/min, gradient condition from 55% B to 85% B). The pure fractions were collected and lyophilized to yield Compound 4 (8.00 mg, 2% yield) as a white powder.

¹H NMR CDCl₃ (Bruker-400 MHz): δ 8.93 (s, 1H), 8.46 (s, 1H), 8.38 (s, 1H), 7.38 (s, 1H), 7.22 (d, J=8.0 Hz, 1H), 7.12 (d, J=6.4 Hz, 1H), 3.38-3.87 (m, 5H), 2.93-3.27 (m, 6H), 2.44 (s, 2H), 2.01 (d, J=7.2 Hz, 3H), 1.33-1.91 (m, 11H), 1.11 (br s, 2H), 0.67-0.99 (m, 8H), 0.30-0.40 (m, 2H)

### Preparation of Compound 5

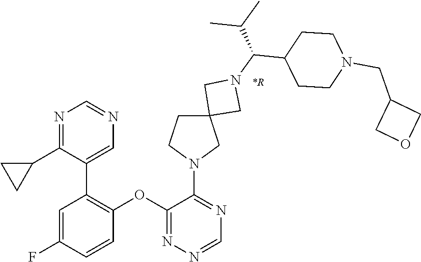 [SMILES: CC(C)[C@H](C1CCN(CC2COC2)CC1)N1CC2(CCN(c3ncnnc3Oc3ccc(F)cc3-c3cncnc3C3CC3)C2)C1]

PyBrOP (108 mg, 0.232 mmol) was added to a solution of intermediate 14 (50 mg; 0.15 mmol), intermediate 28 (61 mg, 0.16 mmol), TEA (0.12 mL, 0.88 mmol), and DMF (0.5 mL). The reaction mixture was stirred at r.t. for 0.5 hours. The mixture was purified by preparative high performance liquid chromatography over Phenomenex Gemini-NX 150×30 mm×5 um (eluent: water (0.04% NH₃H₂O+10 mM NH₄HCO₃)/ACN from 65/35 to 41/59 v/v). The pure fractions were collected and lyophilized to dryness to remove the solvent residue completely to yield Compound 5 (7.64 mg), as a white solid.

¹H NMR CDCl₃ (Varian_400 MHz): δ 8.93 (s, 1H), 8.46 (s, 1H), 8.38 (s, 1H), 7.37 (br. s, 1H), 7.25-7.18 (m, 1H), 7.15-7.09 (m, 1H), 4.83-4.73 (m, 2H), 4.40 (t, J=6.4 Hz, 2H), 3.75-3.42 (m, 4H), 3.41-3.16 (m, 2H), 3.16-3.03 (m, 3H), 3.03-2.93 (m, 1H), 2.81-2.73 (m, 2H), 2.69-2.62 (m, 2H), 2.05-1.99 (m, 2H), 1.88-1.76 (m, 4H), 1.56-1.39 (m, 4H), 1.37-1.28 (m, 1H), 1.16-1.03 (m, 2H), 0.98-0.63 (m, 8H).

### Preparation of Compound 6

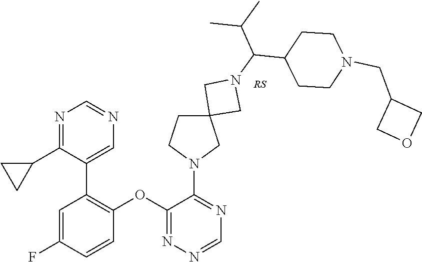 [SMILES: CC(C)C(C1CCN(CC2COC2)CC1)N1CC2(CCN(c3ncnnc3Oc3ccc(F)cc3-c3cncnc3C3CC3)C2)C1]

Sodium cyanoborohydride (30 mg; 0.477 mmol) was added to a mixture of intermediate 33 (100 mg; 0.238 mmol), intermediate 34 (107 mg; 0.477 mmol) and acetic acid (14 μL; 0.238 mmol) in MeOH (5 mL) and the reaction mixture was heated at 70° C. for 60 h. The reaction mixture was cooled to r.t., diluted with DCM and poured onto a 10% aqueous solution of K₂CO₃. The organic layer was decanted, filtered through Chromabond® and evaporated to dryness. The residue (190 mg) was purified by chromatography over silica gel (irregular SiOH, 4 g+4 g; mobile phase: gradient from 0% NH₄OH, 0% MeOH, 100% DCM to 1% NH₄OH, 10% MeOH, 90% DCM). The pure fractions were collected and evaporated to dryness yielding 54 mg of material which was freeze dried (10 mL; 20% ACN, 80% water) to afford 52 mg (35% yield) of Compound 6.

The compounds in the table below were prepared by SFC separation of compound 6

| Compound number | Structure |
|---|---|
| Compound 5 | 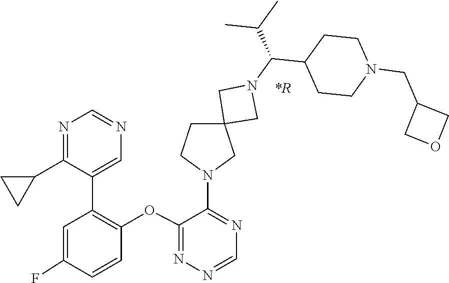 [SMILES: CC(C)[C@H](C1CCN(CC2COC2)CC1)N1CC2(CCN(c3ncnnc3Oc3ccc(F)cc3-c3cncnc3C3CC3)C2)C1] |
| Compound 29 | 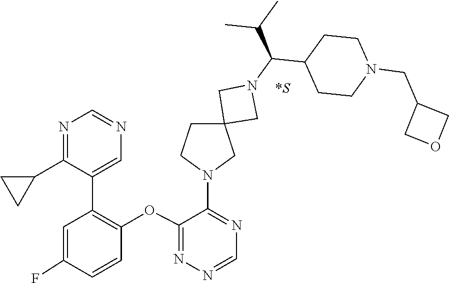 [SMILES: CC(C)[C@@H](C1CCN(CC2COC2)CC1)N1CC2(CCN(c3ncnnc3Oc3ccc(F)cc3-c3cncnc3C3CC3)C2)C1] |
| | Chiral separation (CHIRALPAK AD-H 5 μm 250*30 mm; mobile |
| | phase: 75% CO2, 25% mixture of iPrOH/ACN 80/20 v/v |
| | (+0.3% iPrNH2) |

### Preparation of Compound 7 and Compound 8

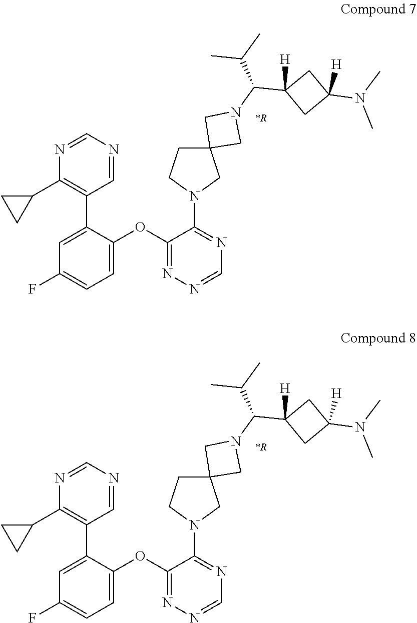 [SMILES: CC(C)[C@H]([C@H]1C[C@@H](N(C)C)C1)N1CC2(CCN(c3ncnnc3Oc3ccc(F)cc3-c3cncnc3C3CC3)C2)C1 ; CC(C)[C@H]([C@H]1C[C@H](N(C)C)C1)N1CC2(CCN(c3ncnnc3Oc3ccc(F)cc3-c3cncnc3C3CC3)C2)C1]

NaBH₃CN (139 mg; 2.21 mmol) was added to a mixture of intermediate 38b (*R) (600 mg; 1.1 mmol), dimethylamine solution (2.76 mL; 5.52 mmol; 2 M in THF), AcOH (63 μL; 1.1 mmol) in MeOH (30 mL). Then, the reaction mixture was heated at 60° C. for 18 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.7% NH₄OH, 7% MeOH, 93% DCM). The pure fractions were collected and evaporated to dryness. The residue (650 mg) was purified by achiral SFC (CHIRALPAK AD-H 5 μm 250*30 mm, mobile phase: 82% CO₂, 18% EtOH (0.3% iPrNH₂)). The pure fractions were collected and the solvent was evaporated to give 425 mg of compound 7 as a white foam and 62 mg of compound 8 as colourless oil. Compound 7 was freeze-dried with water-ACN to give 420 mg (66%) of final compound as a white solid.

### Compound 7

¹H NMR (500 MHz, DMSO-d₆) δ ppm 8.92 (br, 1H) 8.41 (br s, 2H) 7.57 (br s, 1H) 7.46 (br d, J=7.6 Hz, 2H) 3.40-3.79 (m, 4H) 2.89-3.14 (m, 4H) 2.27-2.38 (m, 1H) 2.05 (m, 2H) 1.97 (s, 9H) 1.72-1.87 (m, 2H) 1.61 (m, 2H) 1.45 (q, J=9.6 Hz, 1H) 1.01 (m, 3H) 0.82 (br t, J=6.0 Hz, 7H)

The compounds reported below were prepared following an analogous methodology as reported for the preparation of compound 7 starting from the appropriate starting materials (e.g. intermediate 38 (*R) or other appropriate starting materials):

| Compound number | Structure |
|---|---|
| Compound 30 | 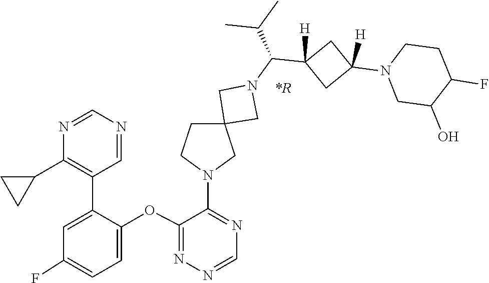 [SMILES: CC(C)[C@H]([C@H]1C[C@@H](N2CCC(F)C(O)C2)C1)N1CC2(CCN(c3ncnnc3Oc3ccc(F)cc3-c3cncnc3C3CC3)C2)C1] |
| Compound 31 | 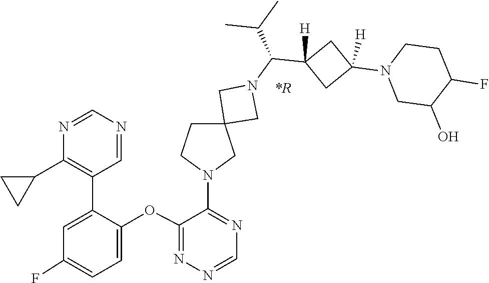 [SMILES: CC(C)[C@H]([C@H]1C[C@H](N2CCC(F)C(O)C2)C1)N1CC2(CCN(c3ncnnc3Oc3ccc(F)cc3-c3cncnc3C3CC3)C2)C1] |
| | In Compounds 30 and 31, the piperidine moiety is one cis |
| | isomer |
| | (undetermined which cis) |
| Compound 34 | 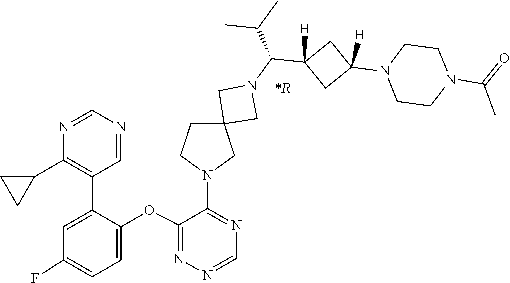 [SMILES: CC(=O)N1CCN([C@H]2C[C@@H]([C@@H](C(C)C)N3CC4(CCN(c5ncnnc5Oc5ccc(F)cc5-c5cncnc5C5CC5)C4)C3)C2)CC1] |
| Compound 35 | 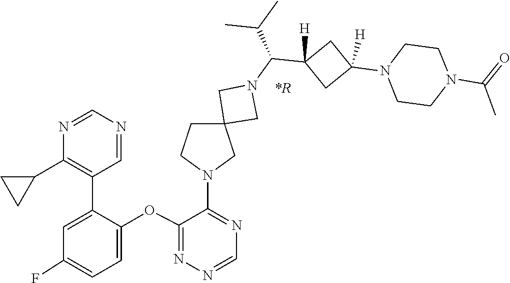 [SMILES: CC(=O)N1CCN([C@H]2C[C@H]([C@@H](C(C)C)N3CC4(CCN(c5ncnnc5Oc5ccc(F)cc5-c5cncnc5C5CC5)C4)C3)C2)CC1] |
| Compound 36 | 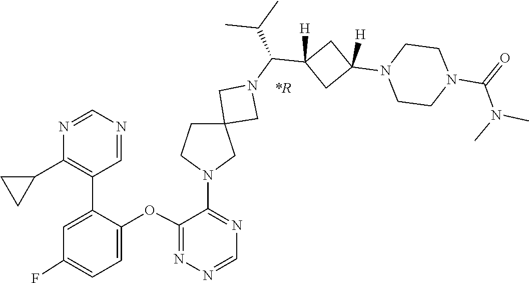 [SMILES: CC(C)[C@H]([C@H]1C[C@@H](N2CCN(C(=O)N(C)C)CC2)C1)N1CC2(CCN(c3ncnnc3Oc3ccc(F)cc3-c3cncnc3C3CC3)C2)C1] |
| Compound 37 | 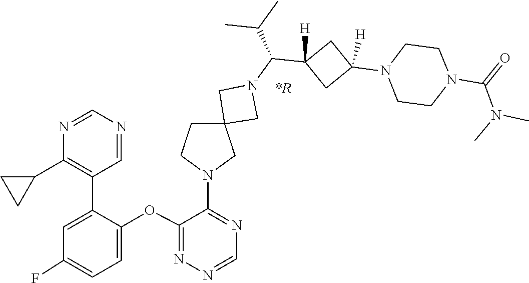 [SMILES: CC(C)[C@H]([C@H]1C[C@H](N2CCN(C(=O)N(C)C)CC2)C1)N1CC2(CCN(c3ncnnc3Oc3ccc(F)cc3-c3cncnc3C3CC3)C2)C1] |
| Compound 38 | 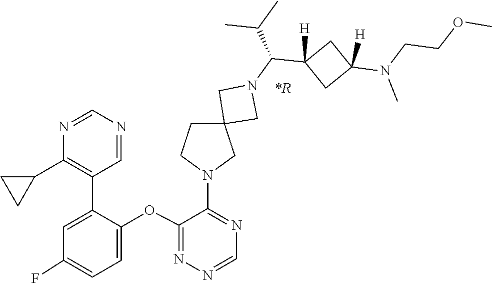 [SMILES: COCCN(C)[C@H]1C[C@@H]([C@@H](C(C)C)N2CC3(CCN(c4ncnnc4Oc4ccc(F)cc4-c4cncnc4C4CC4)C3)C2)C1] |
| Compound 39 | 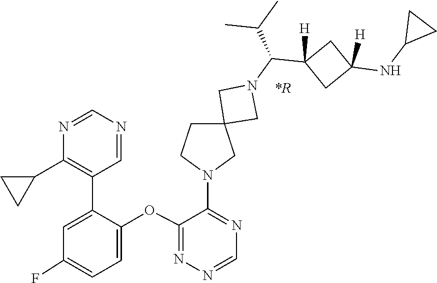 [SMILES: CC(C)[C@H]([C@H]1C[C@@H](NC2CC2)C1)N1CC2(CCN(c3ncnnc3Oc3ccc(F)cc3-c3cncnc3C3CC3)C2)C1] |
| Compound 40 | 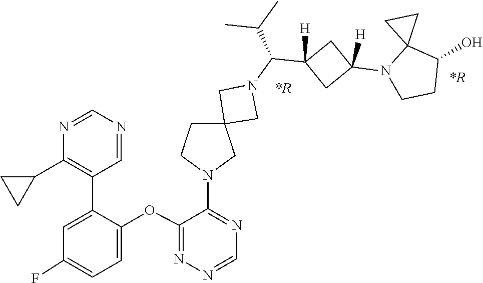 [SMILES: CC(C)[C@H]([C@H]1C[C@@H](N2CC[C@@H](O)C23CC3)C1)N1CC2(CCN(c3ncnnc3Oc3ccc(F)cc3-c3cncnc3C3CC3)C2)C1] |
| Compound 41 | 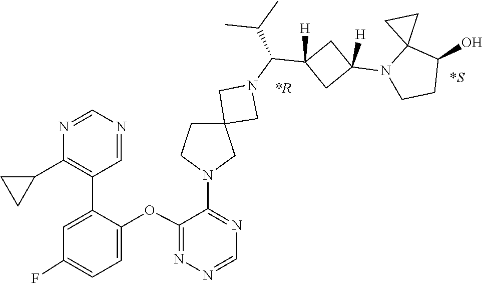 [SMILES: CC(C)[C@H]([C@H]1C[C@@H](N2CC[C@H](O)C23CC3)C1)N1CC2(CCN(c3ncnnc3Oc3ccc(F)cc3-c3cncnc3C3CC3)C2)C1] |
| Compound 42 | 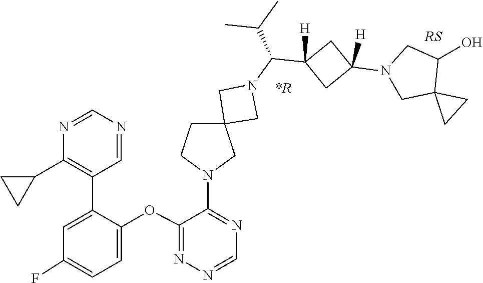 [SMILES: CC(C)[C@H]([C@H]1C[C@@H](N2CC(O)C3(CC3)C2)C1)N1CC2(CCN(c3ncnnc3Oc3ccc(F)cc3-c3cncnc3C3CC3)C2)C1] |
| Compound 43 | 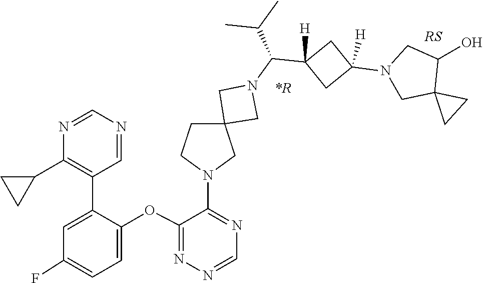 [SMILES: CC(C)[C@H]([C@H]1C[C@H](N2CC(O)C3(CC3)C2)C1)N1CC2(CCN(c3ncnnc3Oc3ccc(F)cc3-c3cncnc3C3CC3)C2)C1] |
| Compound 44 | 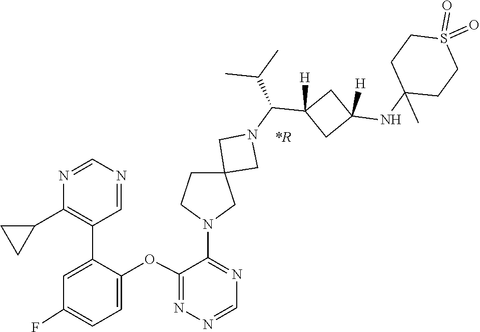 [SMILES: CC(C)[C@H]([C@H]1C[C@@H](NC2(C)CCS(=O)(=O)CC2)C1)N1CC2(CCN(c3ncnnc3Oc3ccc(F)cc3-c3cncnc3C3CC3)C2)C1] |
| Compound 45 | 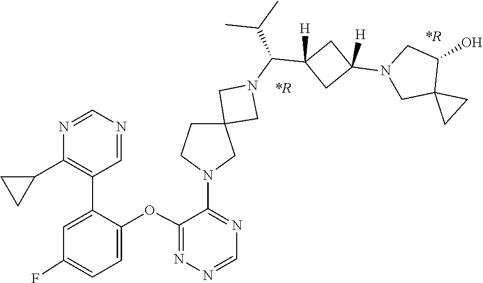 [SMILES: CC(C)[C@H]([C@H]1C[C@@H](N2C[C@H](O)C3(CC3)C2)C1)N1CC2(CCN(c3ncnnc3Oc3ccc(F)cc3-c3cncnc3C3CC3)C2)C1] |
| Compound 46 | 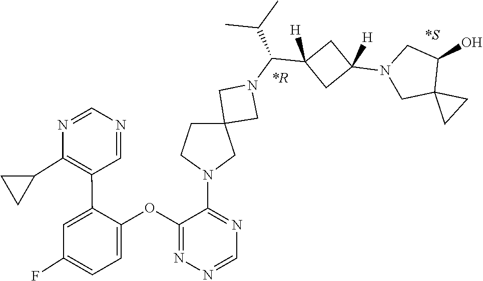 [SMILES: CC(C)[C@H]([C@H]1C[C@@H](N2C[C@@H](O)C3(CC3)C2)C1)N1CC2(CCN(c3ncnnc3Oc3ccc(F)cc3-c3cncnc3C3CC3)C2)C1] |
| | Using BH₃•Pyridine as reductive agent |
| Compound 47 | 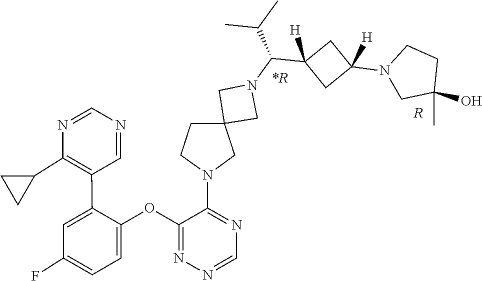 |
| Compound 48 | 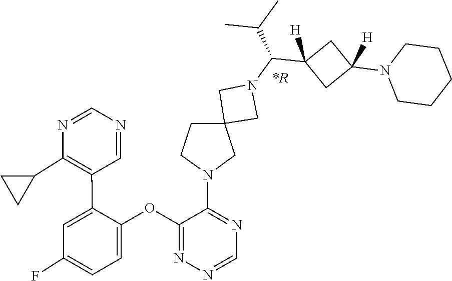 [SMILES: CC(C)[C@H]([C@H]1C[C@@H](N2CCCCC2)C1)N1CC2(CCN(c3ncnnc3Oc3ccc(F)cc3-c3cncnc3C3CC3)C2)C1] |
| Compound 49 | 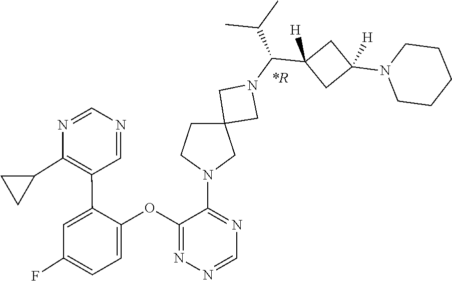 [SMILES: CC(C)[C@H]([C@H]1C[C@H](N2CCCCC2)C1)N1CC2(CCN(c3ncnnc3Oc3ccc(F)cc3-c3cncnc3C3CC3)C2)C1] |
| Compound 50 | 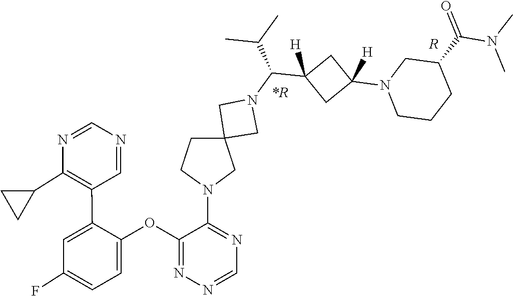 |
| Compound 51 | 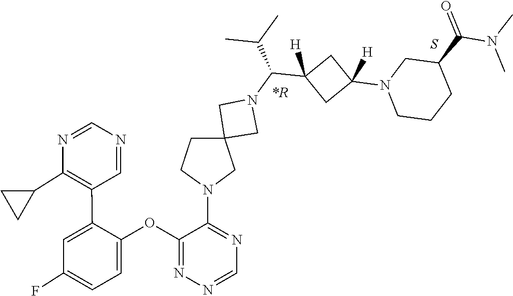 |
| Compound 52 | 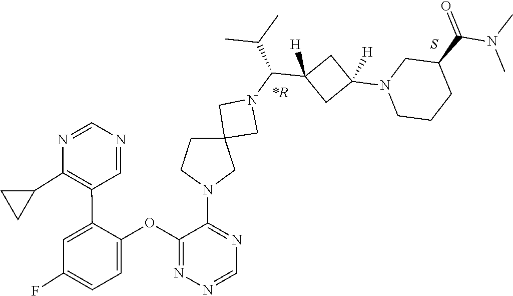 |
| Compound 53 | 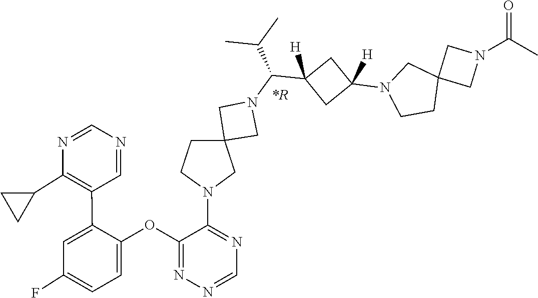 [SMILES: CC(=O)N1CC2(CCN([C@H]3C[C@@H]([C@@H](C(C)C)N4CC5(CCN(c6ncnnc6Oc6ccc(F)cc6-c6cncnc6C6CC6)C5)C4)C3)C2)C1] |
| Compound 54 | 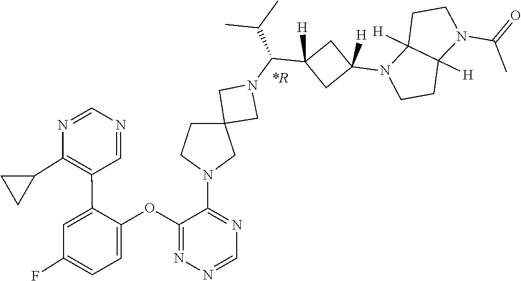 [SMILES: CC(=O)N1CCC2C1CCN2[C@H]1C[C@@H]([C@@H](C(C)C)N2CC3(CCN(c4ncnnc4Oc4ccc(F)cc4-c4cncnc4C4CC4)C3)C2)C1] |
| | Bicyclic moiety is one cis isomer. Undetermined which cis |
| | but the other isomer than Compound 55 |
| Compound 55 | 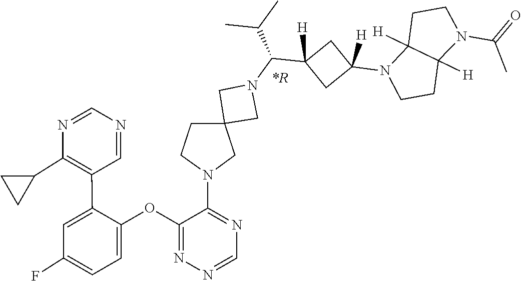 [SMILES: CC(=O)N1CCC2C1CCN2[C@H]1C[C@@H]([C@@H](C(C)C)N2CC3(CCN(c4ncnnc4Oc4ccc(F)cc4-c4cncnc4C4CC4)C3)C2)C1] |
| | Bicyclic moiety is one cis isomer. Undetermined which cis |
| | but the other isomer than Compound 54 |
| Compound 56 | 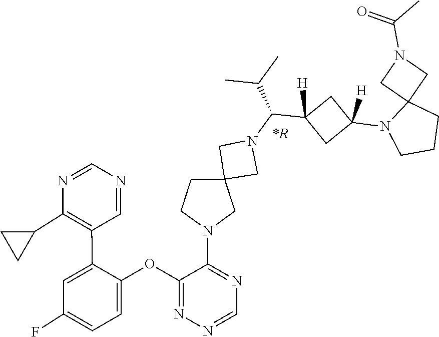 [SMILES: CC(=O)N1CC2(CCCN2[C@H]2C[C@@H]([C@@H](C(C)C)N3CC4(CCN(c5ncnnc5Oc5ccc(F)cc5-c5cncnc5C5CC5)C4)C3)C2)C1] |
| Compound 57 | 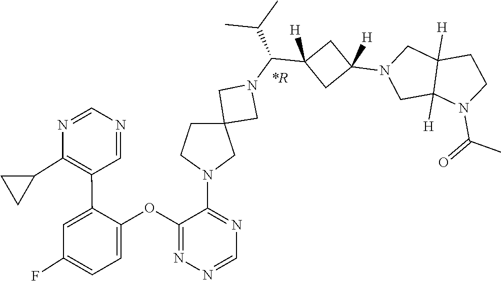 [SMILES: CC(=O)N1CCC2CN([C@H]3C[C@@H]([C@@H](C(C)C)N4CC5(CCN(c6ncnnc6Oc6ccc(F)cc6-c6cncnc6C6CC6)C5)C4)C3)CC21] |
| | Bicyclic moiety is one cis isomer. Undetermined which cis |
| | but the other isomer than Compound 58 |
| Compound 58 | 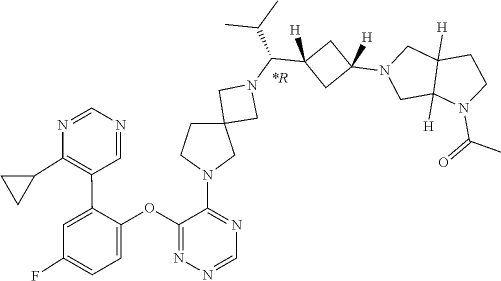 [SMILES: CC(=O)N1CCC2CN([C@H]3C[C@@H]([C@@H](C(C)C)N4CC5(CCN(c6ncnnc6Oc6ccc(F)cc6-c6cncnc6C6CC6)C5)C4)C3)CC21] |
| | Bicyclic moiety is one cis isomer. Undetermined which cis |
| | but the other isomer than Compound 57 |
| Compound 59 | 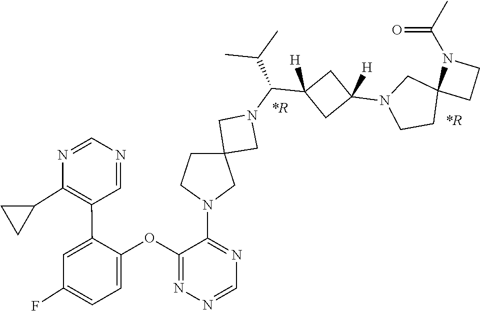 [SMILES: CC(=O)N1CC[C@@]12CCN([C@H]1C[C@@H]([C@@H](C(C)C)N3CC4(CCN(c5ncnnc5Oc5ccc(F)cc5-c5cncnc5C5CC5)C4)C3)C1)C2] |
| Compound 60 | 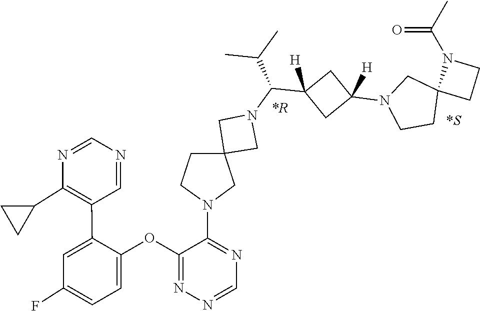 [SMILES: CC(=O)N1CC[C@]12CCN([C@H]1C[C@@H]([C@@H](C(C)C)N3CC4(CCN(c5ncnnc5Oc5ccc(F)cc5-c5cncnc5C5CC5)C4)C3)C1)C2] |
| Compound 61 | 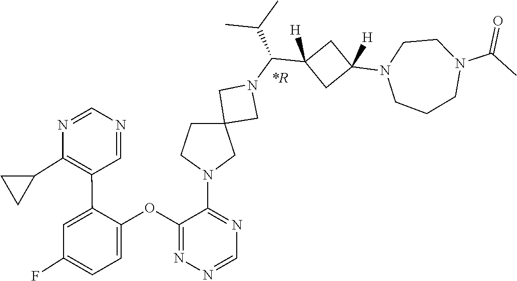 [SMILES: CC(=O)N1CCCN([C@H]2C[C@@H]([C@@H](C(C)C)N3CC4(CCN(c5ncnnc5Oc5ccc(F)cc5-c5cncnc5C5CC5)C4)C3)C2)CC1] |
| Compound 62 | 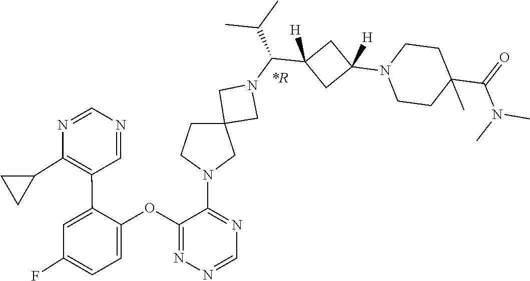 [SMILES: CC(C)[C@H]([C@H]1C[C@@H](N2CCC(C)(C(=O)N(C)C)CC2)C1)N1CC2(CCN(c3ncnnc3Oc3ccc(F)cc3-c3cncnc3C3CC3)C2)C1] |
| Compound 63 | 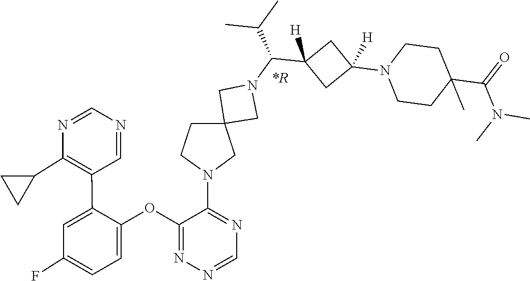 [SMILES: CC(C)[C@H]([C@H]1C[C@H](N2CCC(C)(C(=O)N(C)C)CC2)C1)N1CC2(CCN(c3ncnnc3Oc3ccc(F)cc3-c3cncnc3C3CC3)C2)C1] |
| Compound 64 | 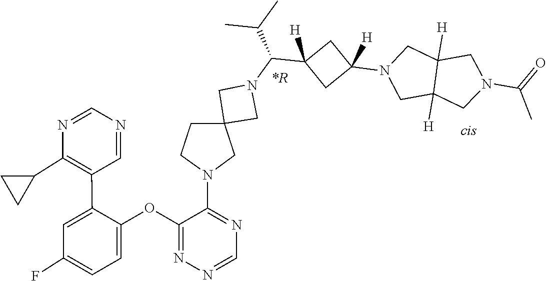 [SMILES: CC(=O)N1CC2CN([C@H]3C[C@@H]([C@@H](C(C)C)N4CC5(CCN(c6ncnnc6Oc6ccc(F)cc6-c6cncnc6C6CC6)C5)C4)C3)CC2C1] |
| Compound 65 | 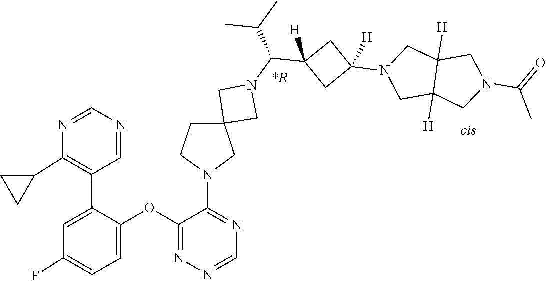 [SMILES: CC(=O)N1CC2CN([C@H]3C[C@H]([C@@H](C(C)C)N4CC5(CCN(c6ncnnc6Oc6ccc(F)cc6-c6cncnc6C6CC6)C5)C4)C3)CC2C1] |
| Compound 66 | 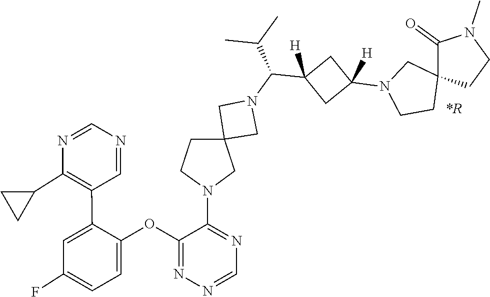 [SMILES: CC(C)[C@H]([C@H]1C[C@@H](N2CC[C@@]3(CCN(C)C3=O)C2)C1)N1CC2(CCN(c3ncnnc3Oc3ccc(F)cc3-c3cncnc3C3CC3)C2)C1] |
| Compound 67 | 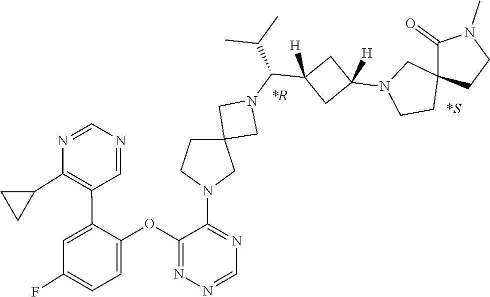 [SMILES: CC(C)[C@H]([C@H]1C[C@@H](N2CC[C@]3(CCN(C)C3=O)C2)C1)N1CC2(CCN(c3ncnnc3Oc3ccc(F)cc3-c3cncnc3C3CC3)C2)C1] |
| Compound 68 | 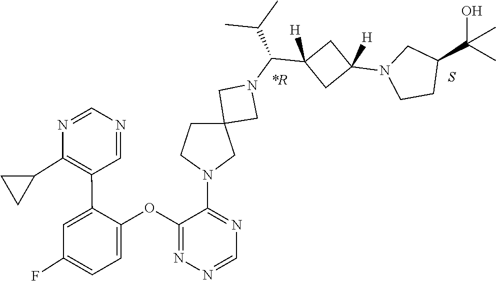 |
| Compound 69 | 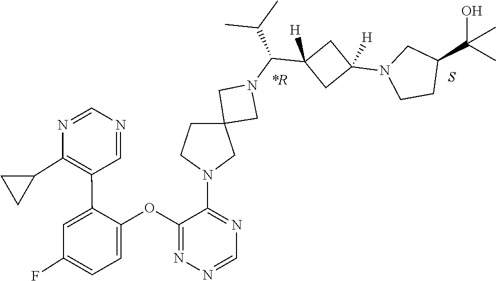 |
| Compound 70 | 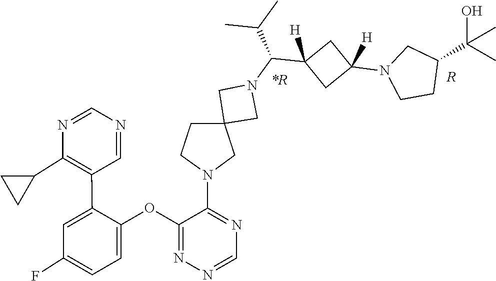 |
| Compound 71 | 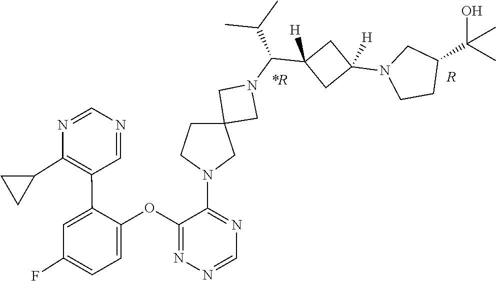 |
| Compound 145 | 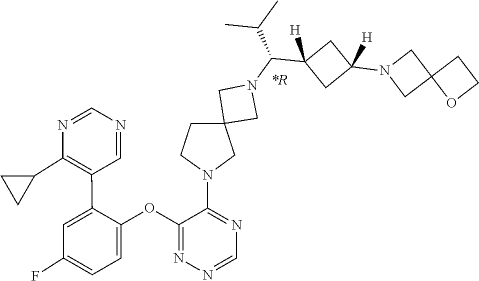 [SMILES: CC(C)[C@H]([C@H]1C[C@@H](N2CC3(CCO3)C2)C1)N1CC2(CCN(c3ncnnc3Oc3ccc(F)cc3-c3cncnc3C3CC3)C2)C1] |
| Compound 146 | 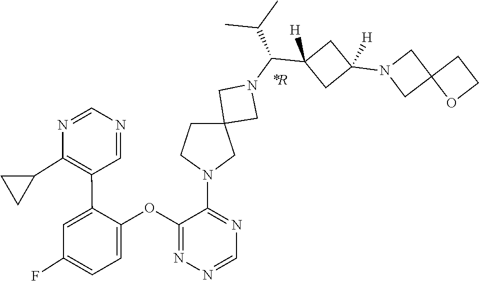 [SMILES: CC(C)[C@H]([C@H]1C[C@H](N2CC3(CCO3)C2)C1)N1CC2(CCN(c3ncnnc3Oc3ccc(F)cc3-c3cncnc3C3CC3)C2)C1] |
| Compound 200 | 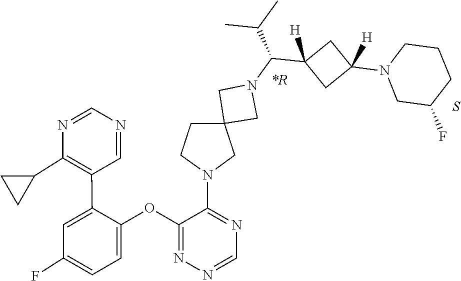 |
| Compound 206 | 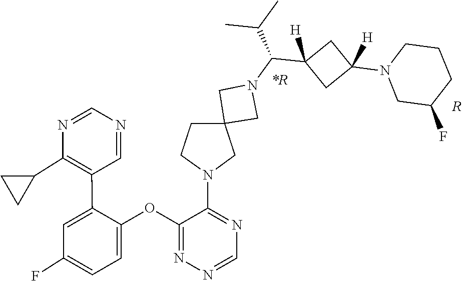 |
| Compound 201 | 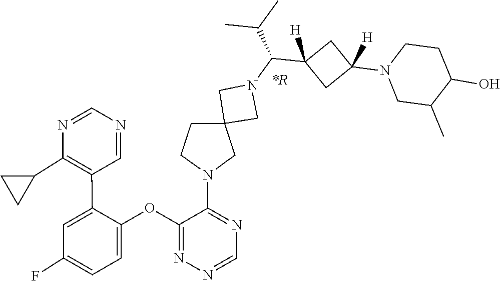 [SMILES: CC1CN([C@H]2C[C@@H]([C@@H](C(C)C)N3CC4(CCN(c5ncnnc5Oc5ccc(F)cc5-c5cncnc5C5CC5)C4)C3)C2)CCC1O] |
| | Piperidine moiety is one cis isomer (undetermined which cis) |
| Compound 207 | 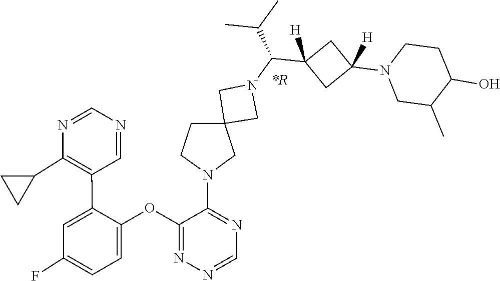 [SMILES: CC1CN([C@H]2C[C@@H]([C@@H](C(C)C)N3CC4(CCN(c5ncnnc5Oc5ccc(F)cc5-c5cncnc5C5CC5)C4)C3)C2)CCC1O] |
| | Piperidine moiety is one trans isomer (8ndetermined which |
| | trans) |
| Compound 204 | 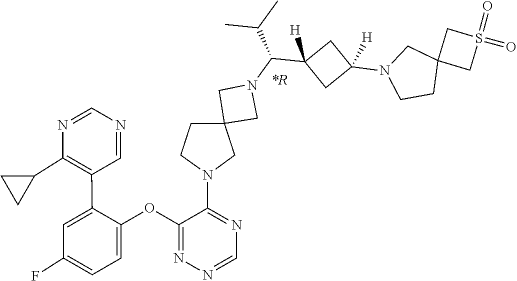 [SMILES: CC(C)[C@H]([C@H]1C[C@H](N2CCC3(C2)CS(=O)(=O)C3)C1)N1CC2(CCN(c3ncnnc3Oc3ccc(F)cc3-c3cncnc3C3CC3)C2)C1] |
| Compound 205 | 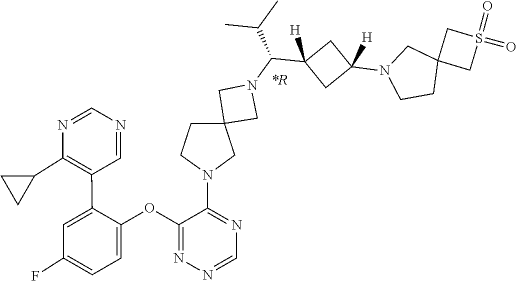 [SMILES: CC(C)[C@H]([C@H]1C[C@@H](N2CCC3(C2)CS(=O)(=O)C3)C1)N1CC2(CCN(c3ncnnc3Oc3ccc(F)cc3-c3cncnc3C3CC3)C2)C1] |
| Compound 208 | 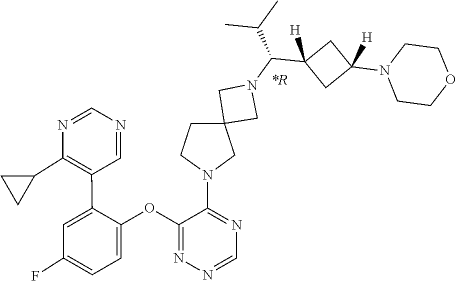 [SMILES: CC(C)[C@H]([C@H]1C[C@@H](N2CCOCC2)C1)N1CC2(CCN(c3ncnnc3Oc3ccc(F)cc3-c3cncnc3C3CC3)C2)C1] |
| Compound 209 | 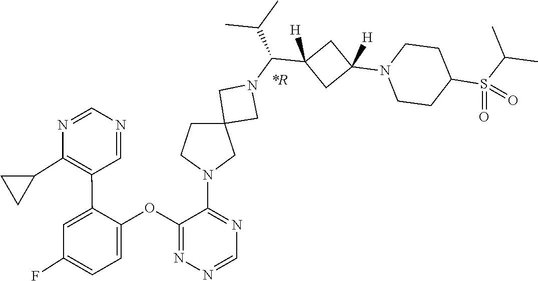 [SMILES: CC(C)[C@H]([C@H]1C[C@@H](N2CCC(S(=O)(=O)C(C)C)CC2)C1)N1CC2(CCN(c3ncnnc3Oc3ccc(F)cc3-c3cncnc3C3CC3)C2)C1] |
| Compound 210 | 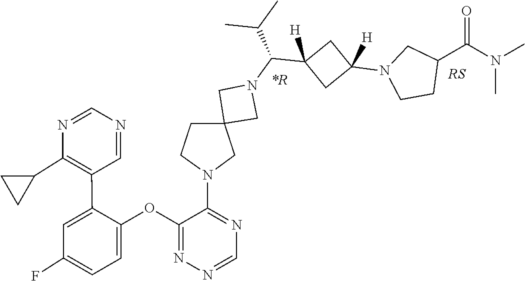 [SMILES: CC(C)[C@H]([C@H]1C[C@@H](N2CCC(C(=O)N(C)C)C2)C1)N1CC2(CCN(c3ncnnc3Oc3ccc(F)cc3-c3cncnc3C3CC3)C2)C1] |
| Compound 211 | 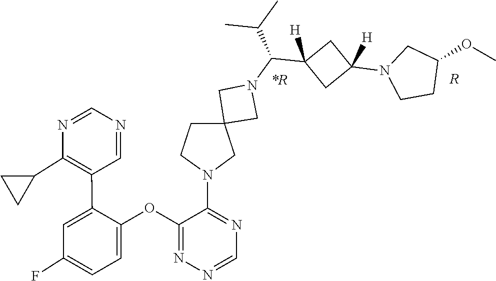 |
| Compound 212 | 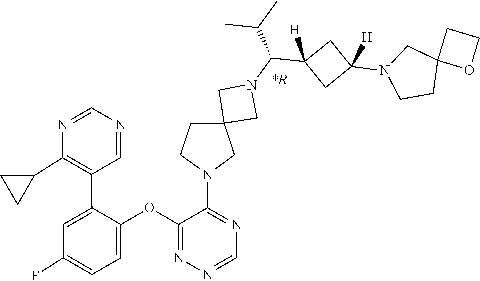 [SMILES: CC(C)[C@H]([C@H]1C[C@@H](N2CCC3(CCO3)C2)C1)N1CC2(CCN(c3ncnnc3Oc3ccc(F)cc3-c3cncnc3C3CC3)C2)C1] |
| Compound 213 | 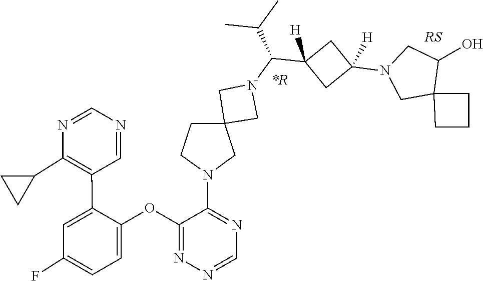 |
| Compound 214 | 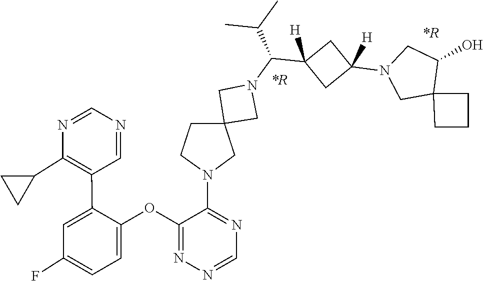 [SMILES: CC(C)[C@H]([C@H]1C[C@@H](N2C[C@H](O)C3(CCC3)C2)C1)N1CC2(CCN(c3ncnnc3Oc3ccc(F)cc3-c3cncnc3C3CC3)C2)C1] |
| Compound 215 | 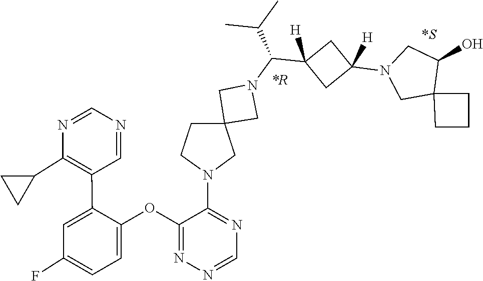 |
| Compound 231 | 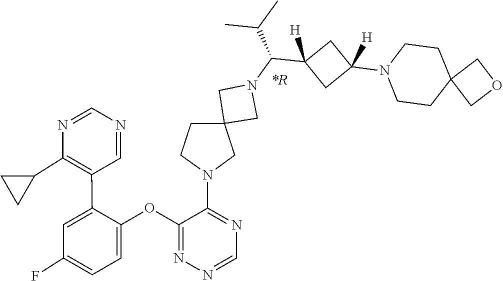 [SMILES: CC(C)[C@H]([C@H]1C[C@@H](N2CCC3(CC2)COC3)C1)N1CC2(CCN(c3ncnnc3Oc3ccc(F)cc3-c3cncnc3C3CC3)C2)C1] |
| Compound 232 | 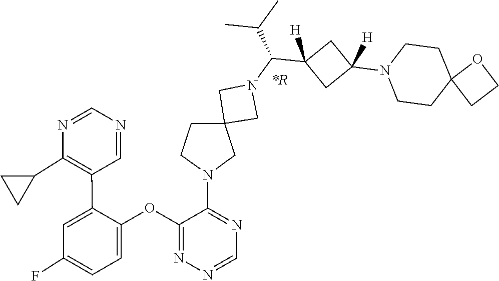 [SMILES: CC(C)[C@H]([C@H]1C[C@@H](N2CCC3(CCO3)CC2)C1)N1CC2(CCN(c3ncnnc3Oc3ccc(F)cc3-c3cncnc3C3CC3)C2)C1] |
| Compound 233 | 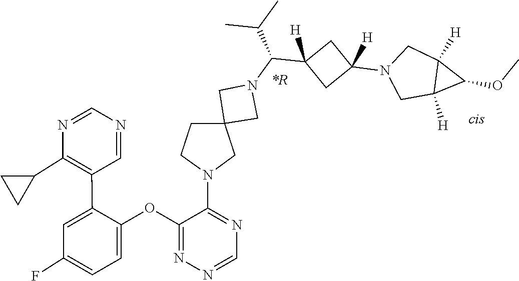 [SMILES: CO[C@H]1[C@@H]2CN([C@H]3C[C@@H]([C@@H](C(C)C)N4CC5(CCN(c6ncnnc6Oc6ccc(F)cc6-c6cncnc6C6CC6)C5)C4)C3)C[C@@H]21] |
| Compound 234 | 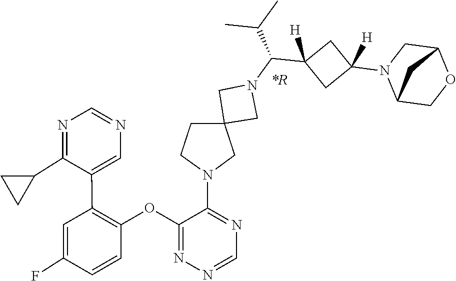 [SMILES: CC(C)[C@H]([C@H]1C[C@@H](N2C[C@H]3C[C@@H]2CO3)C1)N1CC2(CCN(c3ncnnc3Oc3ccc(F)cc3-c3cncnc3C3CC3)C2)C1] |
| Compound 235 | 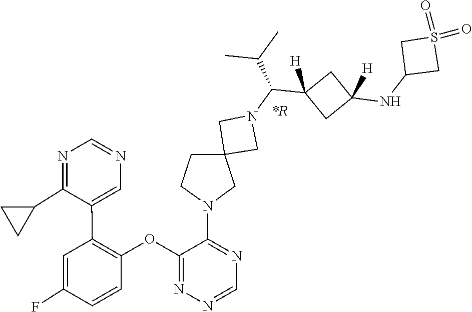 [SMILES: CC(C)[C@H]([C@H]1C[C@@H](NC2CS(=O)(=O)C2)C1)N1CC2(CCN(c3ncnnc3Oc3ccc(F)cc3-c3cncnc3C3CC3)C2)C1] |
| Compound 236 | 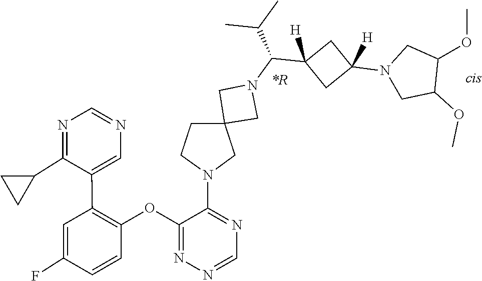 [SMILES: COC1CN([C@H]2C[C@@H]([C@@H](C(C)C)N3CC4(CCN(c5ncnnc5Oc5ccc(F)cc5-c5cncnc5C5CC5)C4)C3)C2)CC1OC] |
| Compound 237 | 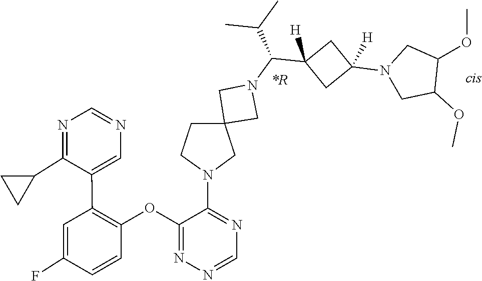 [SMILES: COC1CN([C@H]2C[C@H]([C@@H](C(C)C)N3CC4(CCN(c5ncnnc5Oc5ccc(F)cc5-c5cncnc5C5CC5)C4)C3)C2)CC1OC] |
| Compound 238 | 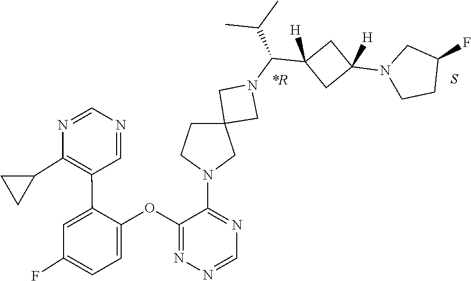 |
| Compound 239 | 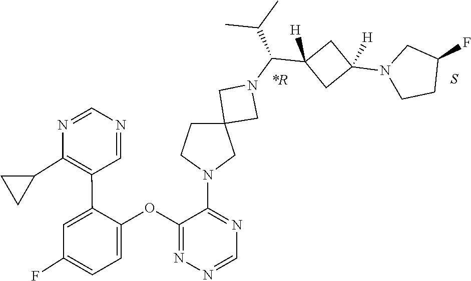 |
| Compound 240 | 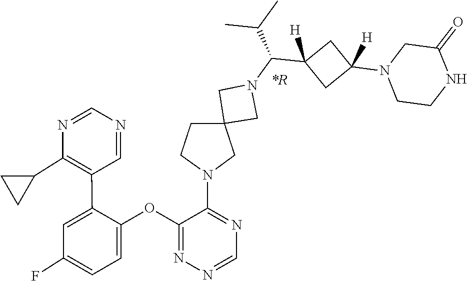 [SMILES: CC(C)[C@H]([C@H]1C[C@@H](N2CCNC(=O)C2)C1)N1CC2(CCN(c3ncnnc3Oc3ccc(F)cc3-c3cncnc3C3CC3)C2)C1] |
| Compound 241 | 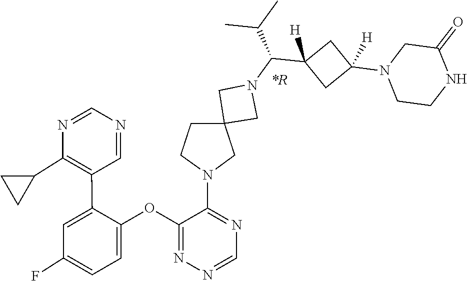 [SMILES: CC(C)[C@H]([C@H]1C[C@H](N2CCNC(=O)C2)C1)N1CC2(CCN(c3ncnnc3Oc3ccc(F)cc3-c3cncnc3C3CC3)C2)C1] |
| Compound 242 | 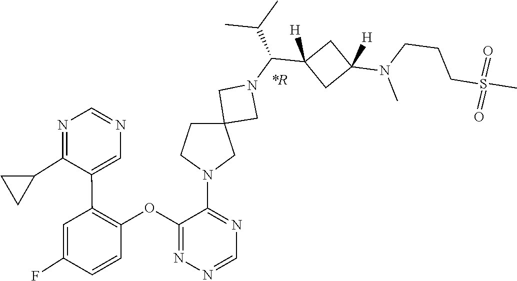 [SMILES: CC(C)[C@H]([C@H]1C[C@@H](N(C)CCCS(C)(=O)=O)C1)N1CC2(CCN(c3ncnnc3Oc3ccc(F)cc3-c3cncnc3C3CC3)C2)C1] |
| Compound 243 | 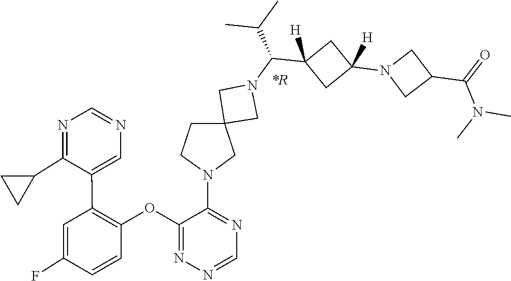 [SMILES: CC(C)[C@H]([C@H]1C[C@@H](N2CC(C(=O)N(C)C)C2)C1)N1CC2(CCN(c3ncnnc3Oc3ccc(F)cc3-c3cncnc3C3CC3)C2)C1] |
| Compound 246 | 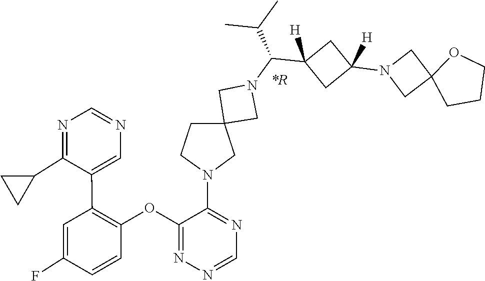 [SMILES: CC(C)[C@H]([C@H]1C[C@@H](N2CC3(CCCO3)C2)C1)N1CC2(CCN(c3ncnnc3Oc3ccc(F)cc3-c3cncnc3C3CC3)C2)C1] |
| Compound 247 | 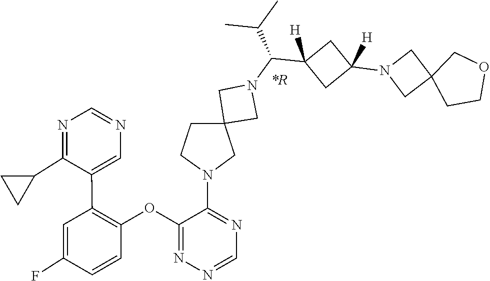 [SMILES: CC(C)[C@H]([C@H]1C[C@@H](N2CC3(CCOC3)C2)C1)N1CC2(CCN(c3ncnnc3Oc3ccc(F)cc3-c3cncnc3C3CC3)C2)C1] |
| Compound 248 | 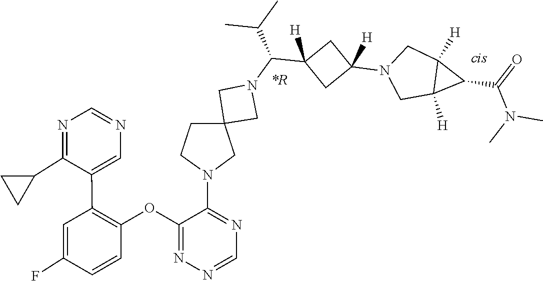 [SMILES: CC(C)[C@H]([C@H]1C[C@@H](N2C[C@@H]3[C@H](C2)[C@H]3C(=O)N(C)C)C1)N1CC2(CCN(c3ncnnc3Oc3ccc(F)cc3-c3cncnc3C3CC3)C2)C1] |
| Compound 249 | 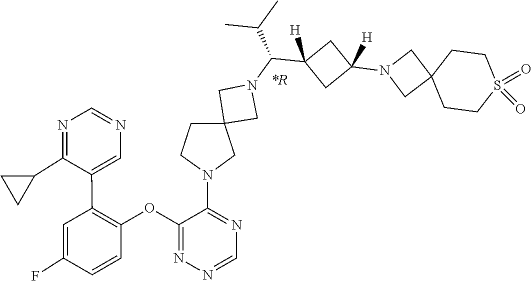 [SMILES: CC(C)[C@H]([C@H]1C[C@@H](N2CC3(CCS(=O)(=O)CC3)C2)C1)N1CC2(CCN(c3ncnnc3Oc3ccc(F)cc3-c3cncnc3C3CC3)C2)C1] |
| Compound 315 | 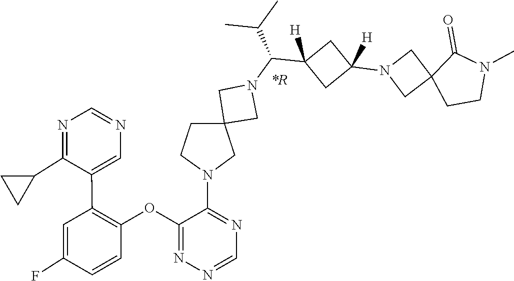 [SMILES: CC(C)[C@H]([C@H]1C[C@@H](N2CC3(CCN(C)C3=O)C2)C1)N1CC2(CCN(c3ncnnc3Oc3ccc(F)cc3-c3cncnc3C3CC3)C2)C1] |
| | from intermediate 238 |
| Compound 316 | 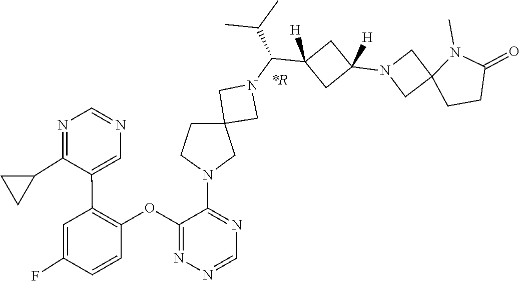 [SMILES: CC(C)[C@H]([C@H]1C[C@@H](N2CC3(CCC(=O)N3C)C2)C1)N1CC2(CCN(c3ncnnc3Oc3ccc(F)cc3-c3cncnc3C3CC3)C2)C1] |
| | from intermediate 240 |
| Compound 317 | 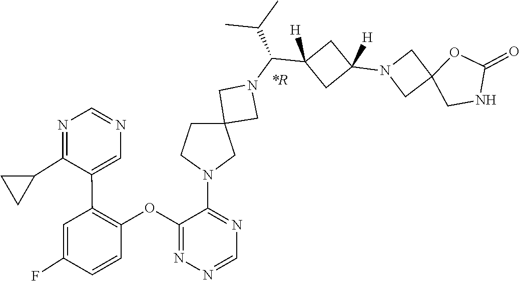 [SMILES: CC(C)[C@H]([C@H]1C[C@@H](N2CC3(CNC(=O)O3)C2)C1)N1CC2(CCN(c3ncnnc3Oc3ccc(F)cc3-c3cncnc3C3CC3)C2)C1] |
| | from intermediate 241 |
| Compound 318 | 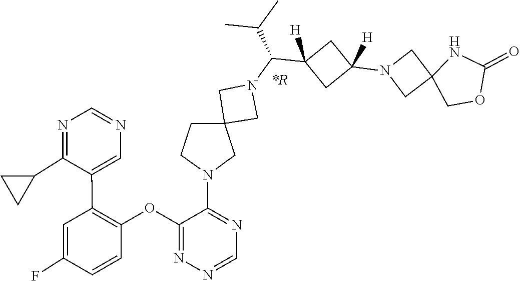 [SMILES: CC(C)[C@H]([C@H]1C[C@@H](N2CC3(COC(=O)N3)C2)C1)N1CC2(CCN(c3ncnnc3Oc3ccc(F)cc3-c3cncnc3C3CC3)C2)C1] |
| | from intermediate 243 |
| Compound 325 | 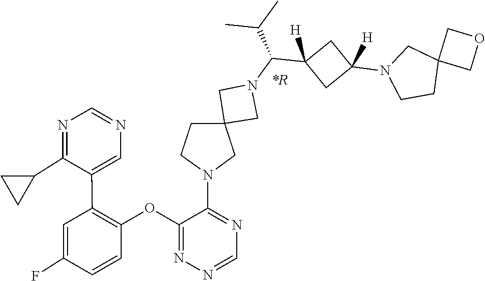 [SMILES: CC(C)[C@H]([C@H]1C[C@@H](N2CCC3(COC3)C2)C1)N1CC2(CCN(c3ncnnc3Oc3ccc(F)cc3-c3cncnc3C3CC3)C2)C1] |
| Compound 329 | 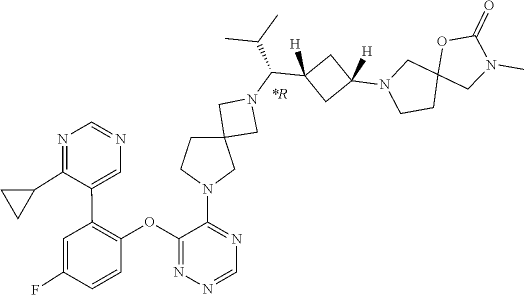 [SMILES: CC(C)[C@H]([C@H]1C[C@@H](N2CCC3(CN(C)C(=O)O3)C2)C1)N1CC2(CCN(c3ncnnc3Oc3ccc(F)cc3-c3cncnc3C3CC3)C2)C1] |
| | from intermediate 248 |
| Compound 345 | 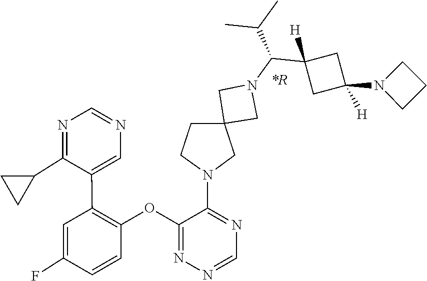 [SMILES: CC(C)[C@H]([C@H]1C[C@H](N2CCC2)C1)N1CC2(CCN(c3ncnnc3Oc3ccc(F)cc3-c3cncnc3C3CC3)C2)C1] |
| Compound 341 | 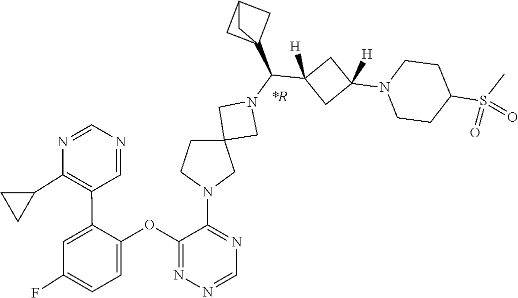 [SMILES: CS(=O)(=O)C1CCN([C@H]2C[C@@H]([C@@H](N3CC4(CCN(c5ncnnc5Oc5ccc(F)cc5-c5cncnc5C5CC5)C4)C3)C34CC(C3)C4)C2)CC1] |
| | from intermediate 295, SFC separation: DAICEL |
| | CHIRALPAK IG (250 mm × 30 mm, 10 um), mobile phase: A: |
| | Supercritical CO₂, B: 0.1% NH₃H₂O EtOH, eluent: isocratic |
| | 60% B v/v, flow rate: 80 mL/min |
| Compound 78 | 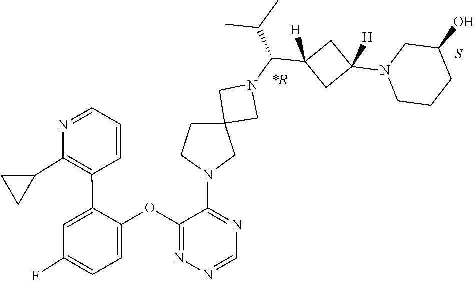 |
| | from intermediate 46a (*R) |
| Compound 79 | 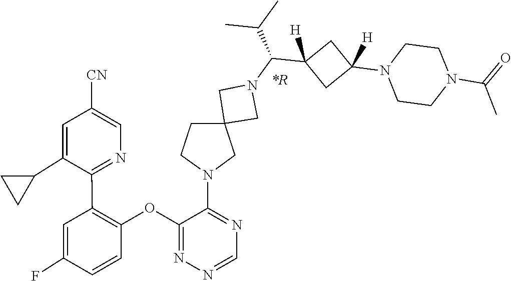 [SMILES: CC(=O)N1CCN([C@H]2C[C@@H]([C@@H](C(C)C)N3CC4(CCN(c5ncnnc5Oc5ccc(F)cc5-c5ncc(C#N)cc5C5CC5)C4)C3)C2)CC1] |
| | from intermediate 82 |
| Compound 80 | 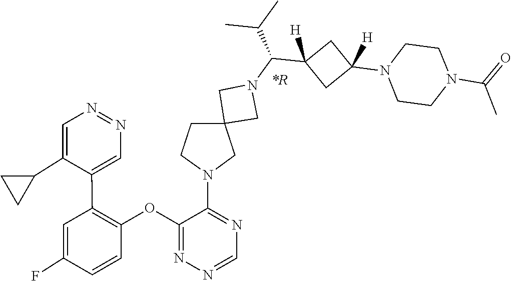 [SMILES: CC(=O)N1CCN([C@H]2C[C@@H]([C@@H](C(C)C)N3CC4(CCN(c5ncnnc5Oc5ccc(F)cc5-c5cnncc5C5CC5)C4)C3)C2)CC1] |
| | from intermediate 109 |
| Compound 81 | 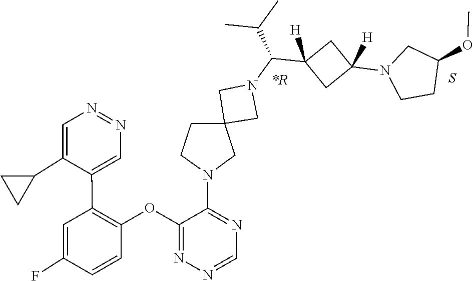 |
| | from intermediate 109 |
| Compound 82 | 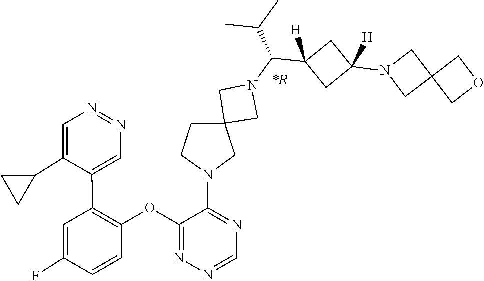 [SMILES: CC(C)[C@H]([C@H]1C[C@@H](N2CC3(COC3)C2)C1)N1CC2(CCN(c3ncnnc3Oc3ccc(F)cc3-c3cnncc3C3CC3)C2)C1] |
| | compound 82 |
| Compound 83 | 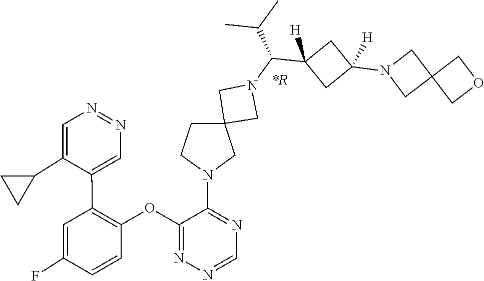 [SMILES: CC(C)[C@H]([C@H]1C[C@H](N2CC3(COC3)C2)C1)N1CC2(CCN(c3ncnnc3Oc3ccc(F)cc3-c3cnncc3C3CC3)C2)C1] |
| | from intermediate 109 |
| Compound 84 | 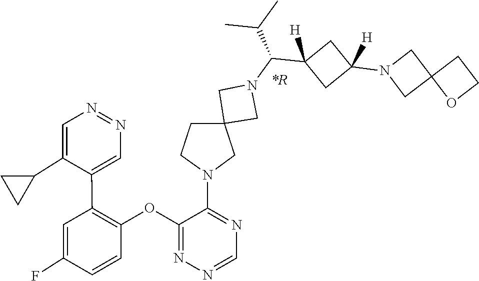 [SMILES: CC(C)[C@H]([C@H]1C[C@@H](N2CC3(CCO3)C2)C1)N1CC2(CCN(c3ncnnc3Oc3ccc(F)cc3-c3cnncc3C3CC3)C2)C1] |
| Compound 84a | 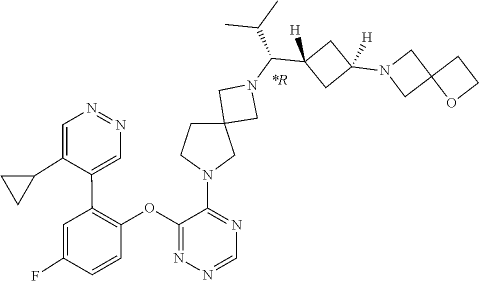 [SMILES: CC(C)[C@H]([C@H]1C[C@H](N2CC3(CCO3)C2)C1)N1CC2(CCN(c3ncnnc3Oc3ccc(F)cc3-c3cnncc3C3CC3)C2)C1] |
| | from intermediate 109 |

The compounds reported below were prepared following an analogous methodology as reported for the preparation of compound 7, starting from the appropriate starting materials (e.g. intermediate 38a (*S) or other appropriate starting materials):

| Compound number | Structure |
|---|---|
| Compound 85 | 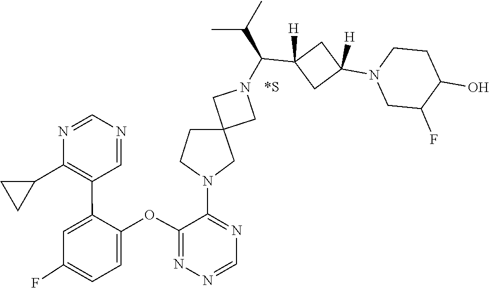 [SMILES: CC(C)[C@@H]([C@H]1C[C@@H](N2CCC(O)C(F)C2)C1)N1CC2(CCN(c3ncnnc3Oc3ccc(F)cc3-c3cncnc3C3CC3)C2)C1] |
| | Piperidine moiety is one cis isomer (undetermined which cis) |
| Compound 86 | 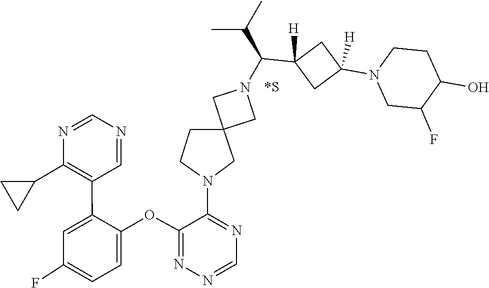 [SMILES: CC(C)[C@@H]([C@H]1C[C@H](N2CCC(O)C(F)C2)C1)N1CC2(CCN(c3ncnnc3Oc3ccc(F)cc3-c3cncnc3C3CC3)C2)C1] |
| | Piperidine moiety is one cis isomer (undetermined which cis) |
| Compound 87 | 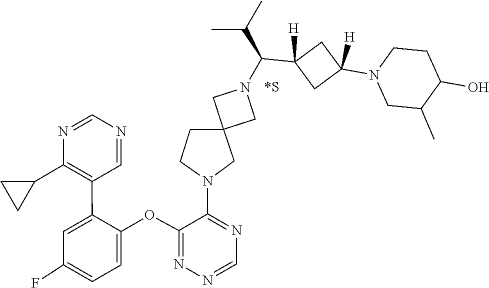 [SMILES: CC1CN([C@H]2C[C@@H]([C@H](C(C)C)N3CC4(CCN(c5ncnnc5Oc5ccc(F)cc5-c5cncnc5C5CC5)C4)C3)C2)CCC1O] |
| | Piperidine moiety is one cis isomer (undetermined which cis) |
| Compound 88 | 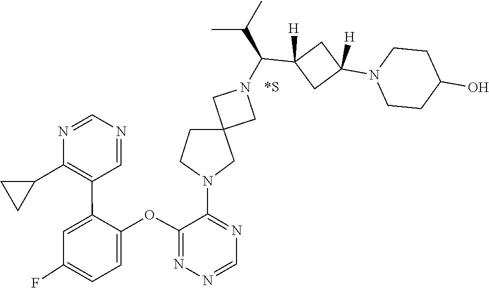 [SMILES: CC(C)[C@@H]([C@H]1C[C@@H](N2CCC(O)CC2)C1)N1CC2(CCN(c3ncnnc3Oc3ccc(F)cc3-c3cncnc3C3CC3)C2)C1] |
| Compound 244 | 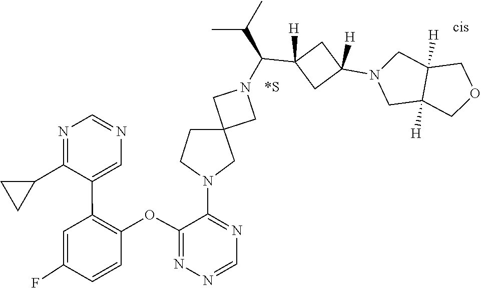 [SMILES: CC(C)[C@@H]([C@H]1C[C@@H](N2C[C@H]3COC[C@H]3C2)C1)N1CC2(CCN(c3ncnnc3Oc3ccc(F)cc3-c3cncnc3C3CC3)C2)C1] |
| Compound 245 | 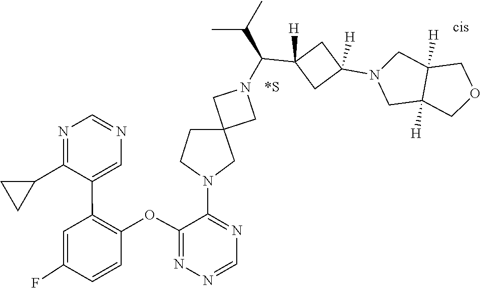 [SMILES: CC(C)[C@@H]([C@H]1C[C@H](N2C[C@H]3COC[C@H]3C2)C1)N1CC2(CCN(c3ncnnc3Oc3ccc(F)cc3-c3cncnc3C3CC3)C2)C1] |
| Compound 346 | 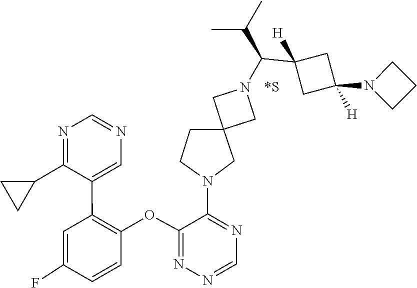 [SMILES: CC(C)[C@@H]([C@H]1C[C@H](N2CCC2)C1)N1CC2(CCN(c3ncnnc3Oc3ccc(F)cc3-c3cncnc3C3CC3)C2)C1] |
| Compound 89 | 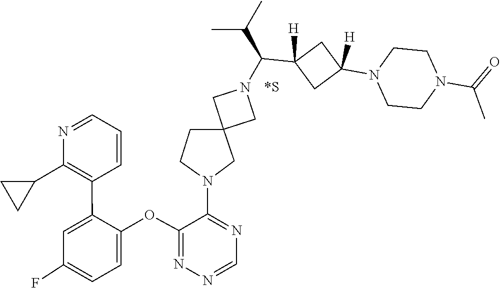 [SMILES: CC(=O)N1CCN([C@H]2C[C@@H]([C@H](C(C)C)N3CC4(CCN(c5ncnnc5Oc5ccc(F)cc5-c5cccnc5C5CC5)C4)C3)C2)CC1] |
| | from intermediate 46b (*S) |
| Compound 90 | 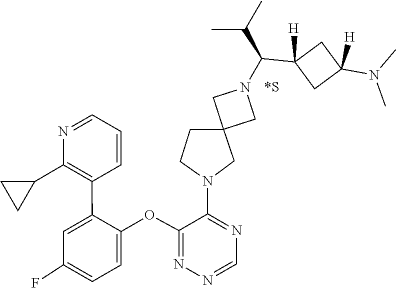 [SMILES: CC(C)[C@@H]([C@H]1C[C@@H](N(C)C)C1)N1CC2(CCN(c3ncnnc3Oc3ccc(F)cc3-c3cccnc3C3CC3)C2)C1] |
| Compound 91 | 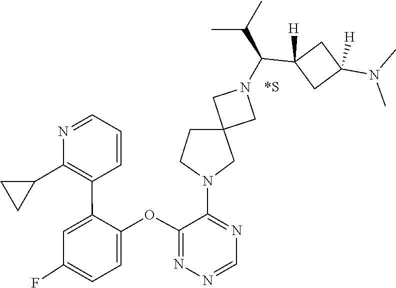 [SMILES: CC(C)[C@@H]([C@H]1C[C@H](N(C)C)C1)N1CC2(CCN(c3ncnnc3Oc3ccc(F)cc3-c3cccnc3C3CC3)C2)C1] |
| | from intermediate 46b (*S) |
| Compound 92 | 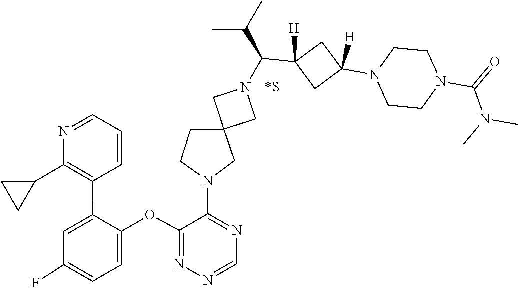 [SMILES: CC(C)[C@@H]([C@H]1C[C@@H](N2CCN(C(=O)N(C)C)CC2)C1)N1CC2(CCN(c3ncnnc3Oc3ccc(F)cc3-c3cccnc3C3CC3)C2)C1] |
| | from intermediate 46b (*S) |
| Compound 93 | 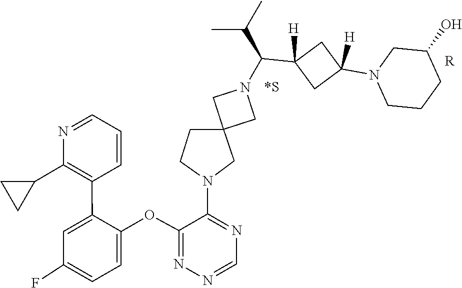 |
| | from intermediate 46b (*S) |
| Compound 94 | 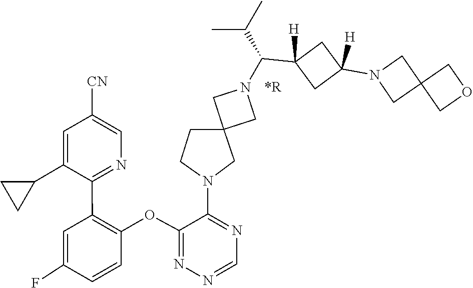 [SMILES: CC(C)[C@H]([C@H]1C[C@@H](N2CC3(COC3)C2)C1)N1CC2(CCN(c3ncnnc3Oc3ccc(F)cc3-c3ncc(C#N)cc3C3CC3)C2)C1] |
| Compound 95 | 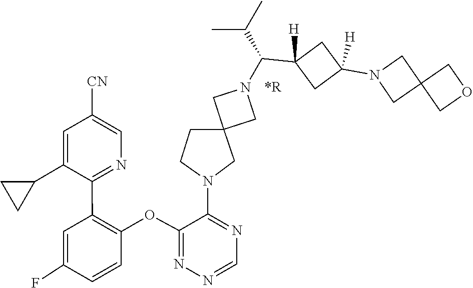 [SMILES: CC(C)[C@H]([C@H]1C[C@H](N2CC3(COC3)C2)C1)N1CC2(CCN(c3ncnnc3Oc3ccc(F)cc3-c3ncc(C#N)cc3C3CC3)C2)C1] |
| | from intermediate 82 |
| Compound 96 | 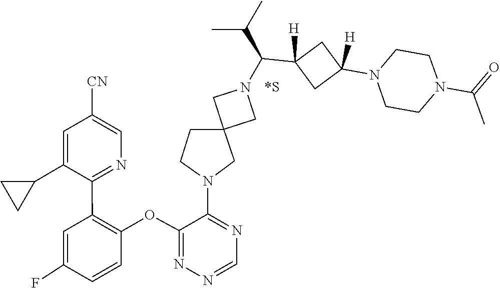 [SMILES: CC(=O)N1CCN([C@H]2C[C@@H]([C@H](C(C)C)N3CC4(CCN(c5ncnnc5Oc5ccc(F)cc5-c5ncc(C#N)cc5C5CC5)C4)C3)C2)CC1] |
| | from intermediate 82 |
| Compound 97 | 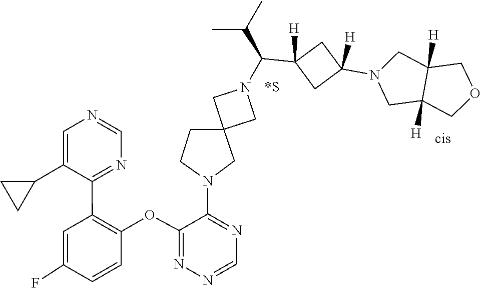 [SMILES: CC(C)[C@@H]([C@H]1C[C@@H](N2C[C@H]3COC[C@H]3C2)C1)N1CC2(CCN(c3ncnnc3Oc3ccc(F)cc3-c3ncncc3C3CC3)C2)C1] |
| | from intermediate 93 |
| Compound 98 | 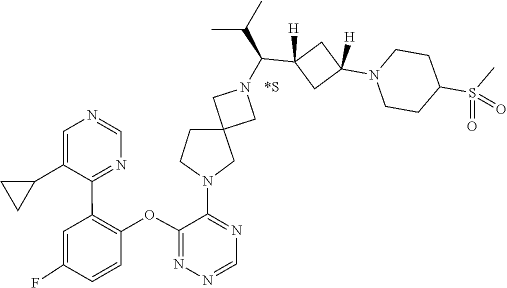 [SMILES: CC(C)[C@@H]([C@H]1C[C@@H](N2CCC(S(C)(=O)=O)CC2)C1)N1CC2(CCN(c3ncnnc3Oc3ccc(F)cc3-c3ncncc3C3CC3)C2)C1] |
| | from intermediate 93 |
| Compound 99 | 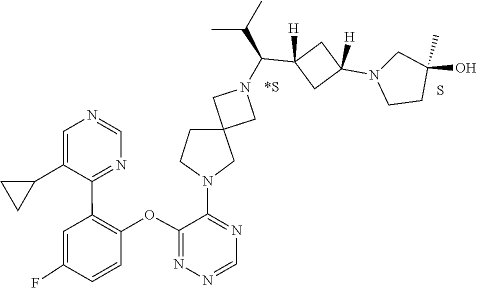 |
| | from intermediate 93 |
| Compound 100 | 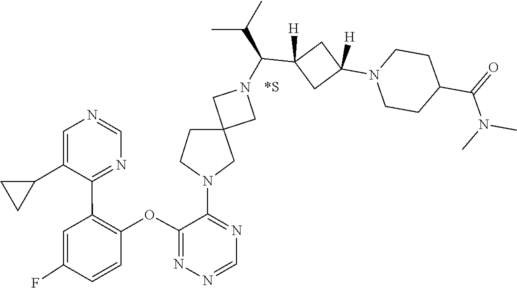 [SMILES: CC(C)[C@@H]([C@H]1C[C@@H](N2CCC(C(=O)N(C)C)CC2)C1)N1CC2(CCN(c3ncnnc3Oc3ccc(F)cc3-c3ncncc3C3CC3)C2)C1] |
| | from intermediate 93 |
| Compound 101 | 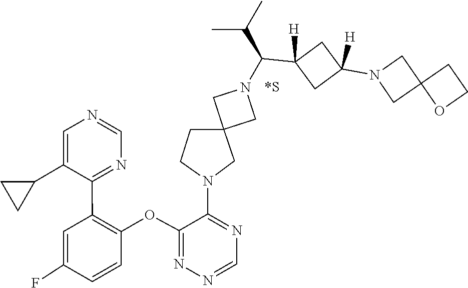 [SMILES: CC(C)[C@@H]([C@H]1C[C@@H](N2CC3(CCO3)C2)C1)N1CC2(CCN(c3ncnnc3Oc3ccc(F)cc3-c3ncncc3C3CC3)C2)C1] |
| | from intermediate 93 |
| Compound 102 | 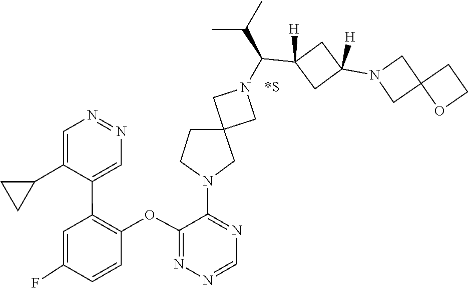 [SMILES: CC(C)[C@@H]([C@H]1C[C@@H](N2CC3(CCO3)C2)C1)N1CC2(CCN(c3ncnnc3Oc3ccc(F)cc3-c3cnncc3C3CC3)C2)C1] |
| | from intermediate 109 |
| Compound 103 | 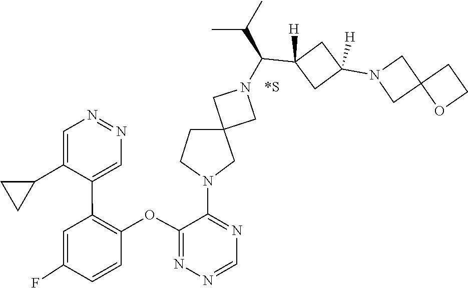 [SMILES: CC(C)[C@@H]([C@H]1C[C@H](N2CC3(CCO3)C2)C1)N1CC2(CCN(c3ncnnc3Oc3ccc(F)cc3-c3cnncc3C3CC3)C2)C1] |
| | from intermediate 109 |
| Compound 104 | 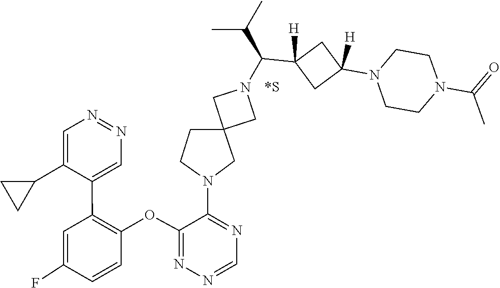 [SMILES: CC(=O)N1CCN([C@H]2C[C@@H]([C@H](C(C)C)N3CC4(CCN(c5ncnnc5Oc5ccc(F)cc5-c5cnncc5C5CC5)C4)C3)C2)CC1] |
| Compound 105 | 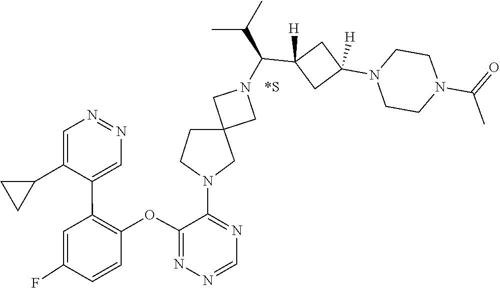 [SMILES: CC(=O)N1CCN([C@H]2C[C@H]([C@H](C(C)C)N3CC4(CCN(c5ncnnc5Oc5ccc(F)cc5-c5cnncc5C5CC5)C4)C3)C2)CC1] |
| | from intermediate 109 |
| Compound 106 | 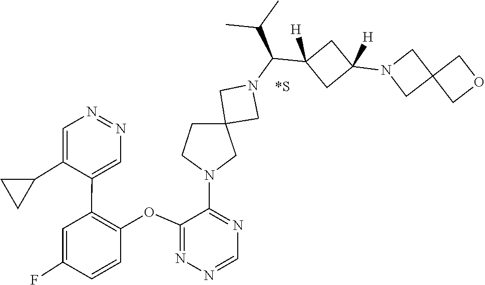 [SMILES: CC(C)[C@@H]([C@H]1C[C@@H](N2CC3(COC3)C2)C1)N1CC2(CCN(c3ncnnc3Oc3ccc(F)cc3-c3cnncc3C3CC3)C2)C1] |
| Compound 107 | 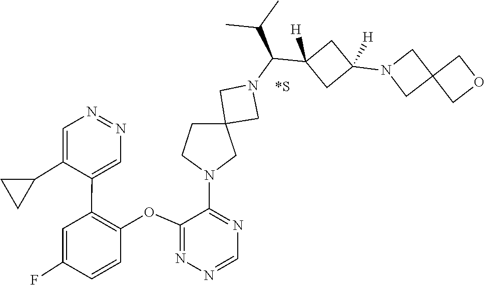 [SMILES: CC(C)[C@@H]([C@H]1C[C@H](N2CC3(COC3)C2)C1)N1CC2(CCN(c3ncnnc3Oc3ccc(F)cc3-c3cnncc3C3CC3)C2)C1] |
| | from intermediate 109 |
| Compound 108 | 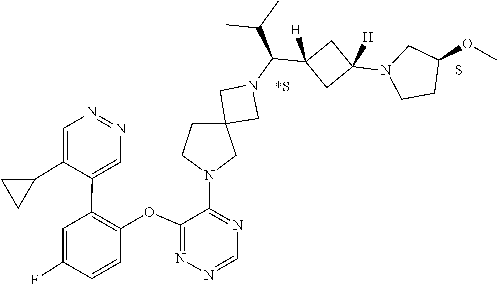 |
| Compound 109 | 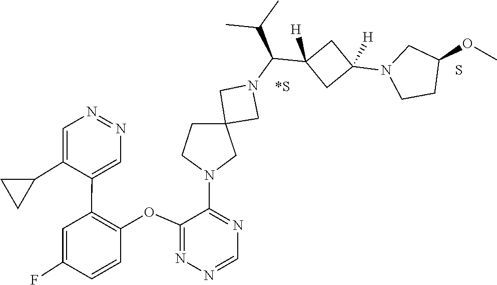 |
| | from intermediate 109 |
| Compound 110 | 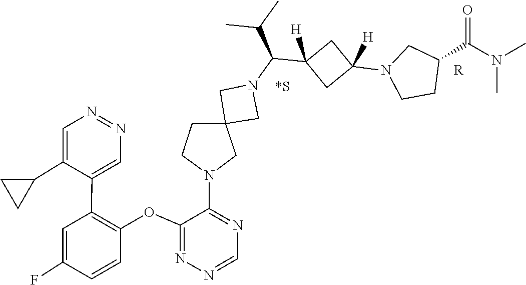 |
| Compound 111 | 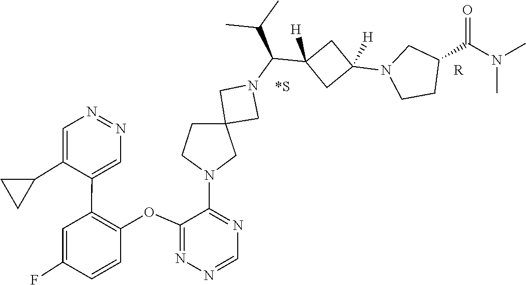 |
| | from intermediate 109 |
| Compound 112 | 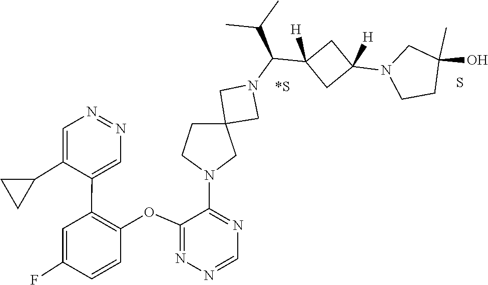 |
| Compound 113 | 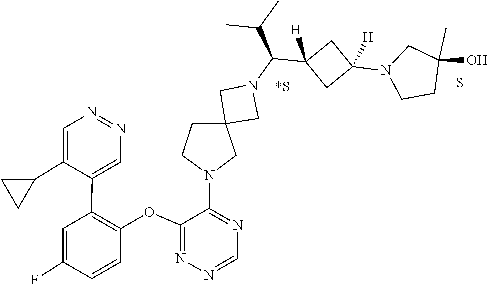 |
| | from intermediate 109 |
| Compound 114 | 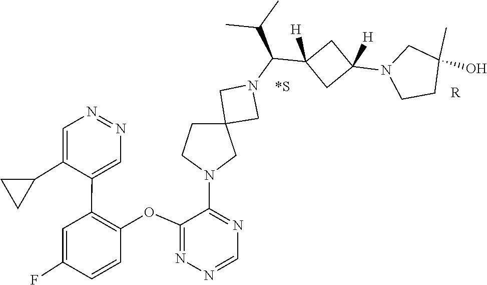 |
| Compound 115 | 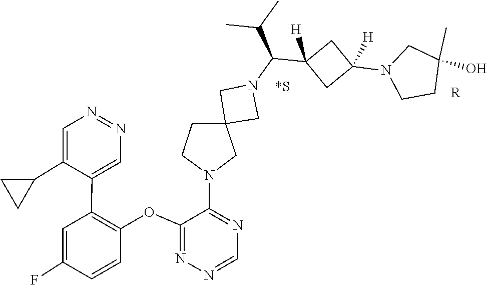 |
| | from intermediate 109 |
| Compound 116 | 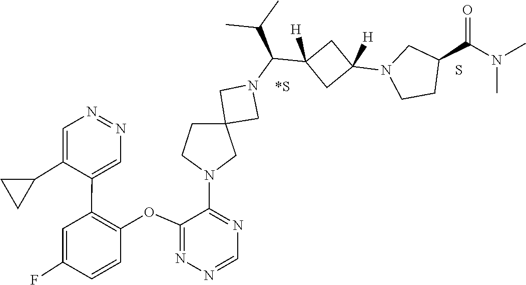 |
| Compound 117 | 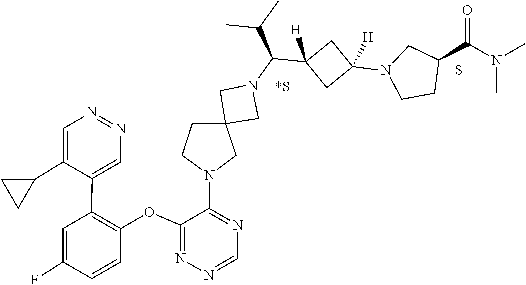 |
| | from intermediate 109 |
| Compound 278 | 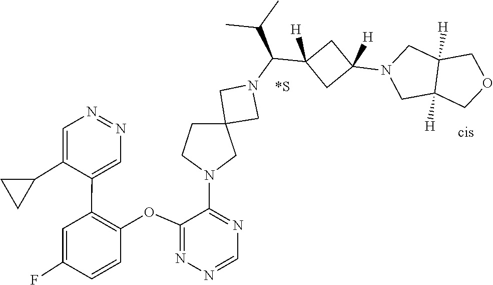 [SMILES: CC(C)[C@@H]([C@H]1C[C@@H](N2C[C@H]3COC[C@H]3C2)C1)N1CC2(CCN(c3ncnnc3Oc3ccc(F)cc3-c3cnncc3C3CC3)C2)C1] |
| Compound 279 | 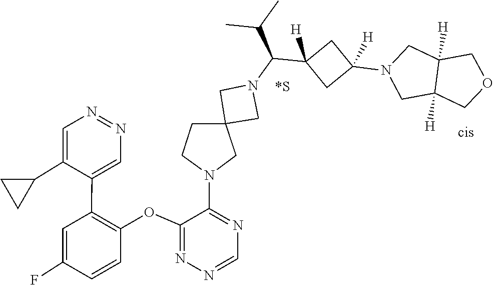 [SMILES: CC(C)[C@@H]([C@H]1C[C@H](N2C[C@H]3COC[C@H]3C2)C1)N1CC2(CCN(c3ncnnc3Oc3ccc(F)cc3-c3cnncc3C3CC3)C2)C1] |
| | from intermediate 109 |
| Compound 282a | 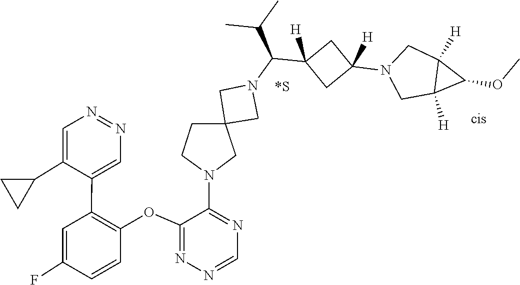 [SMILES: CO[C@H]1[C@@H]2CN([C@H]3C[C@@H]([C@H](C(C)C)N4CC5(CCN(c6ncnnc6Oc6ccc(F)cc6-c6cnncc6C6CC6)C5)C4)C3)C[C@@H]21] |
| Compound 282b | 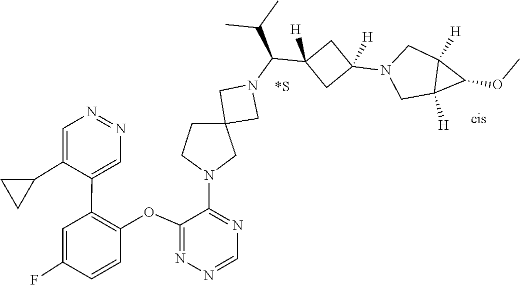 [SMILES: CO[C@H]1[C@@H]2CN([C@H]3C[C@H]([C@H](C(C)C)N4CC5(CCN(c6ncnnc6Oc6ccc(F)cc6-c6cnncc6C6CC6)C5)C4)C3)C[C@@H]21] |
| | from intermediate 109 |
| Compound 280a | 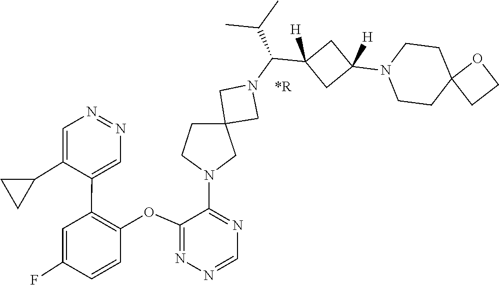 [SMILES: CC(C)[C@H]([C@H]1C[C@@H](N2CCC3(CCO3)CC2)C1)N1CC2(CCN(c3ncnnc3Oc3ccc(F)cc3-c3cnncc3C3CC3)C2)C1] |
| Compound 280b | 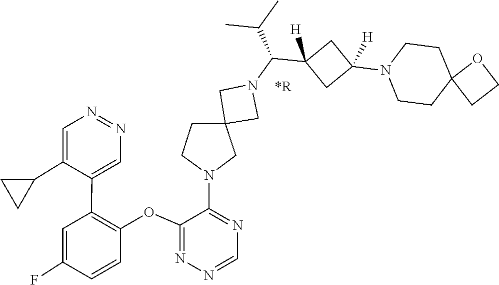 [SMILES: CC(C)[C@H]([C@H]1C[C@H](N2CCC3(CCO3)CC2)C1)N1CC2(CCN(c3ncnnc3Oc3ccc(F)cc3-c3cnncc3C3CC3)C2)C1] |
| Compound 281a | 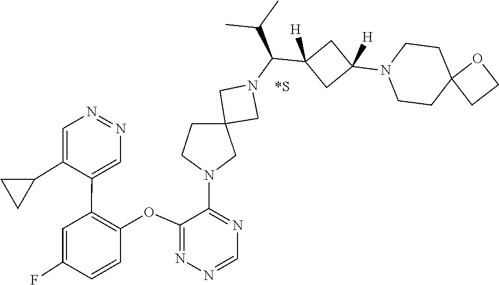 [SMILES: CC(C)[C@@H]([C@H]1C[C@@H](N2CCC3(CCO3)CC2)C1)N1CC2(CCN(c3ncnnc3Oc3ccc(F)cc3-c3cnncc3C3CC3)C2)C1] |
| Compound 281b | 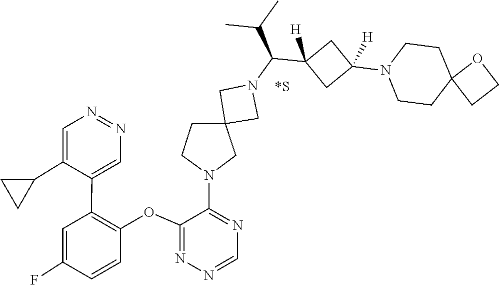 [SMILES: CC(C)[C@@H]([C@H]1C[C@H](N2CCC3(CCO3)CC2)C1)N1CC2(CCN(c3ncnnc3Oc3ccc(F)cc3-c3cnncc3C3CC3)C2)C1] |
| | from intermediate 109 |
| Compound 283a | 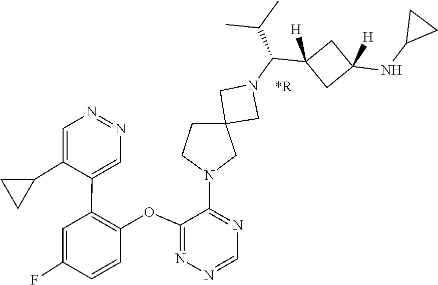 [SMILES: CC(C)[C@H]([C@H]1C[C@@H](NC2CC2)C1)N1CC2(CCN(c3ncnnc3Oc3ccc(F)cc3-c3cnncc3C3CC3)C2)C1] |
| Compound 283b | 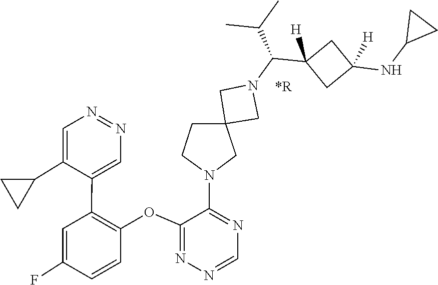 [SMILES: CC(C)[C@H]([C@H]1C[C@H](NC2CC2)C1)N1CC2(CCN(c3ncnnc3Oc3ccc(F)cc3-c3cnncc3C3CC3)C2)C1] |
| Compound 284a | 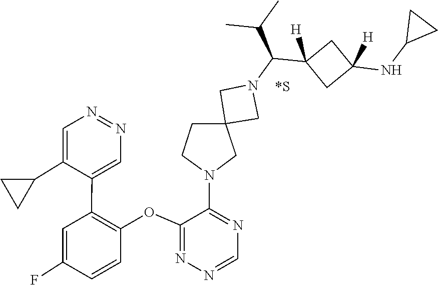 [SMILES: CC(C)[C@@H]([C@H]1C[C@@H](NC2CC2)C1)N1CC2(CCN(c3ncnnc3Oc3ccc(F)cc3-c3cnncc3C3CC3)C2)C1] |
| Compound 284b | 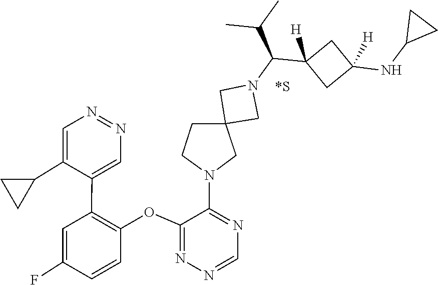 [SMILES: CC(C)[C@@H]([C@H]1C[C@H](NC2CC2)C1)N1CC2(CCN(c3ncnnc3Oc3ccc(F)cc3-c3cnncc3C3CC3)C2)C1] |
| | from intermediate 109 |

The compounds reported below were prepared following an analogous methodology as reported for the preparation of compound 7 starting from the appropriate starting materials (e.g. intermediate 38b or other appropriate starting materials). If needed, standard cleavage of protected groups was applied:

| Compound number | Structure |
|---|---|
| Compound 120 | 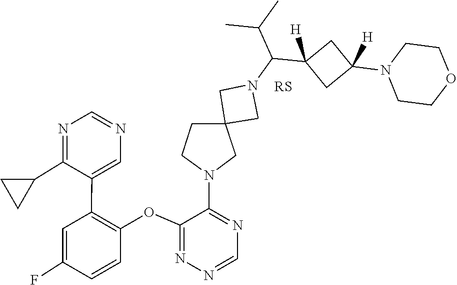 [SMILES: CC(C)C([C@H]1C[C@@H](N2CCOCC2)C1)N1CC2(CCN(c3ncnnc3Oc3ccc(F)cc3-c3cncnc3C3CC3)C2)C1] |
| Compound 121 | 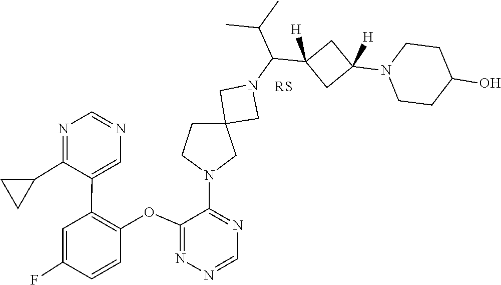 [SMILES: CC(C)C([C@H]1C[C@@H](N2CCC(O)CC2)C1)N1CC2(CCN(c3ncnnc3Oc3ccc(F)cc3-c3cncnc3C3CC3)C2)C1] |
| | Deprotection TBAF 1M (3eq) in THF at RT for 18h. |
| Compound 122 | 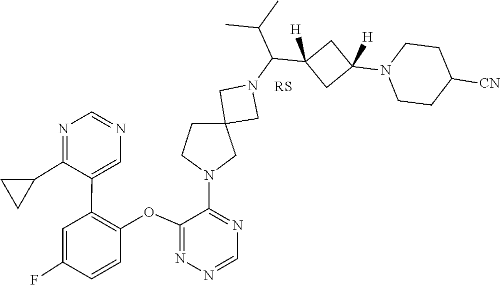 [SMILES: CC(C)C([C@H]1C[C@@H](N2CCC(C#N)CC2)C1)N1CC2(CCN(c3ncnnc3Oc3ccc(F)cc3-c3cncnc3C3CC3)C2)C1] |
| Compound 123 | 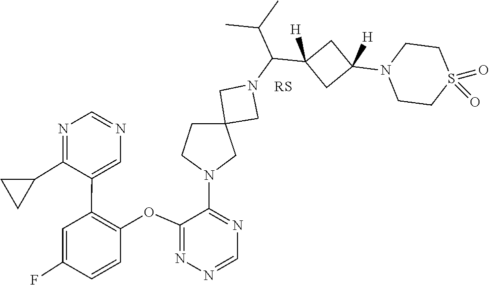 [SMILES: CC(C)C([C@H]1C[C@@H](N2CCS(=O)(=O)CC2)C1)N1CC2(CCN(c3ncnnc3Oc3ccc(F)cc3-c3cncnc3C3CC3)C2)C1] |
| Compound 124 | 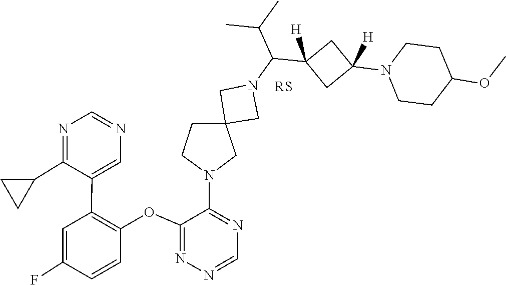 [SMILES: COC1CCN([C@H]2C[C@@H](C(C(C)C)N3CC4(CCN(c5ncnnc5Oc5ccc(F)cc5-c5cncnc5C5CC5)C4)C3)C2)CC1] |
| Compound 125 | 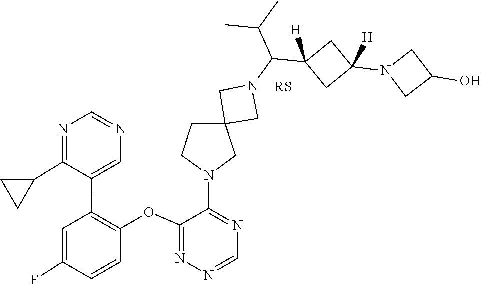 [SMILES: CC(C)C([C@H]1C[C@@H](N2CC(O)C2)C1)N1CC2(CCN(c3ncnnc3Oc3ccc(F)cc3-c3cncnc3C3CC3)C2)C1] |
| Compound 126 | 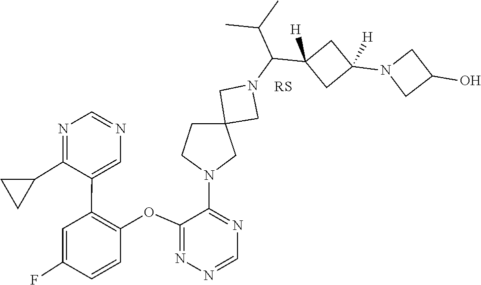 [SMILES: CC(C)C([C@H]1C[C@H](N2CC(O)C2)C1)N1CC2(CCN(c3ncnnc3Oc3ccc(F)cc3-c3cncnc3C3CC3)C2)C1] |
| Compound 127 | 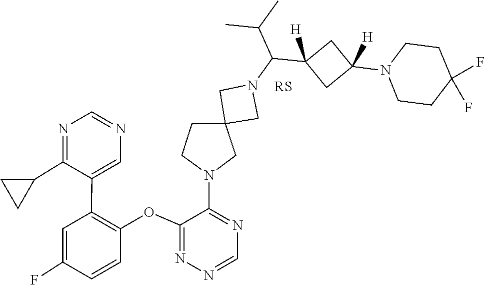 [SMILES: CC(C)C([C@H]1C[C@@H](N2CCC(F)(F)CC2)C1)N1CC2(CCN(c3ncnnc3Oc3ccc(F)cc3-c3cncnc3C3CC3)C2)C1] |
| Compound 128 | 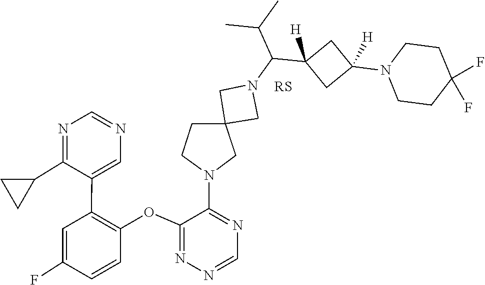 [SMILES: CC(C)C([C@H]1C[C@H](N2CCC(F)(F)CC2)C1)N1CC2(CCN(c3ncnnc3Oc3ccc(F)cc3-c3cncnc3C3CC3)C2)C1] |
| Compound 129 | 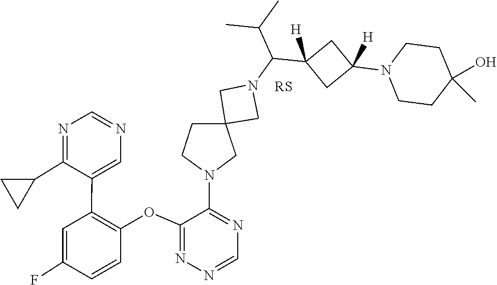 [SMILES: CC(C)C([C@H]1C[C@@H](N2CCC(C)(O)CC2)C1)N1CC2(CCN(c3ncnnc3Oc3ccc(F)cc3-c3cncnc3C3CC3)C2)C1] |
| Compound 130 | 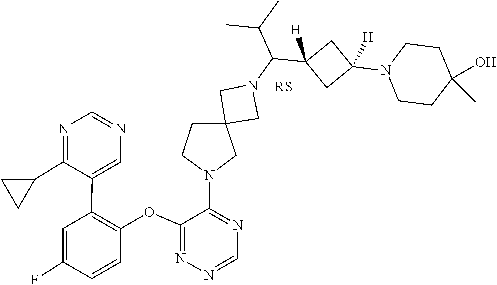 [SMILES: CC(C)C([C@H]1C[C@H](N2CCC(C)(O)CC2)C1)N1CC2(CCN(c3ncnnc3Oc3ccc(F)cc3-c3cncnc3C3CC3)C2)C1] |
| Compound 131 | 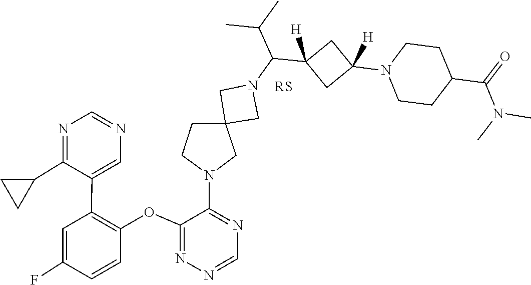 [SMILES: CC(C)C([C@H]1C[C@@H](N2CCC(C(=O)N(C)C)CC2)C1)N1CC2(CCN(c3ncnnc3Oc3ccc(F)cc3-c3cncnc3C3CC3)C2)C1] |
| Compound 132 | 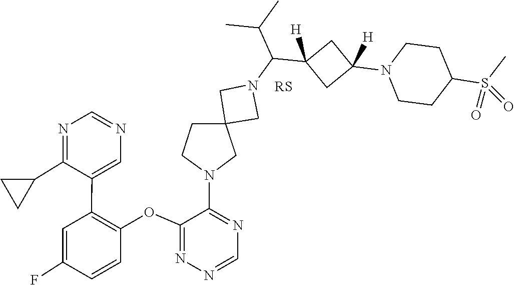 [SMILES: CC(C)C([C@H]1C[C@@H](N2CCC(S(C)(=O)=O)CC2)C1)N1CC2(CCN(c3ncnnc3Oc3ccc(F)cc3-c3cncnc3C3CC3)C2)C1] |
| Compound 133 | 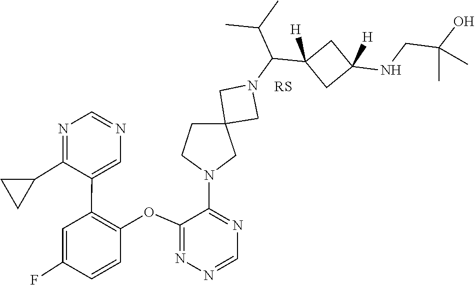 [SMILES: CC(C)C([C@H]1C[C@@H](NCC(C)(C)O)C1)N1CC2(CCN(c3ncnnc3Oc3ccc(F)cc3-c3cncnc3C3CC3)C2)C1] |
| Compound 134 | 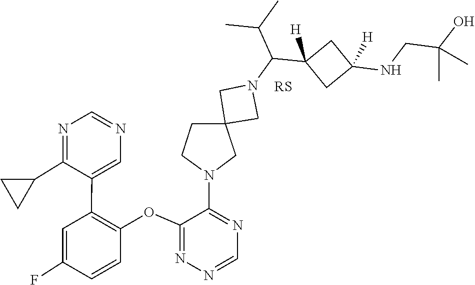 [SMILES: CC(C)C([C@H]1C[C@H](NCC(C)(C)O)C1)N1CC2(CCN(c3ncnnc3Oc3ccc(F)cc3-c3cncnc3C3CC3)C2)C1] |
| Compound 135 | 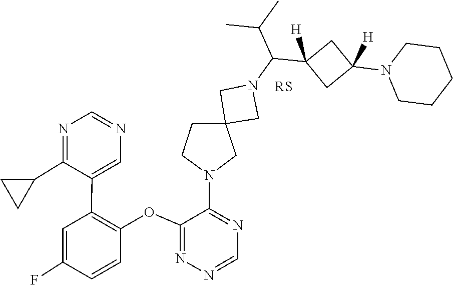 [SMILES: CC(C)C([C@H]1C[C@@H](N2CCCCC2)C1)N1CC2(CCN(c3ncnnc3Oc3ccc(F)cc3-c3cncnc3C3CC3)C2)C1] |
| Compound 136 | 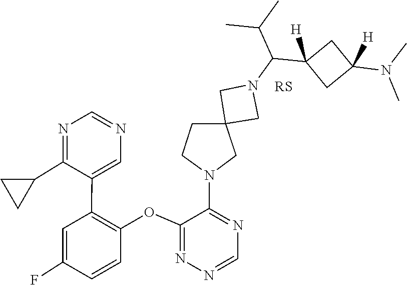 [SMILES: CC(C)C([C@H]1C[C@@H](N(C)C)C1)N1CC2(CCN(c3ncnnc3Oc3ccc(F)cc3-c3cncnc3C3CC3)C2)C1] |
| Compound 326 | 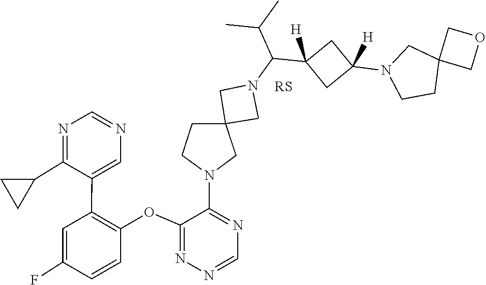 [SMILES: CC(C)C([C@H]1C[C@@H](N2CCC3(COC3)C2)C1)N1CC2(CCN(c3ncnnc3Oc3ccc(F)cc3-c3cncnc3C3CC3)C2)C1] |
| Compound 327 | 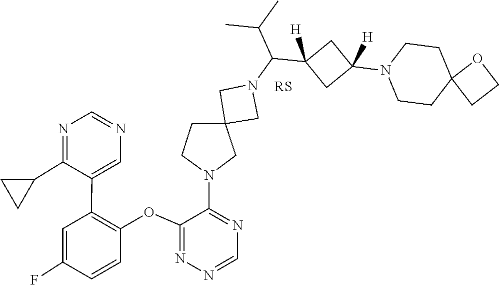 [SMILES: CC(C)C([C@H]1C[C@@H](N2CCC3(CCO3)CC2)C1)N1CC2(CCN(c3ncnnc3Oc3ccc(F)cc3-c3cncnc3C3CC3)C2)C1] |
| Compound 137 | 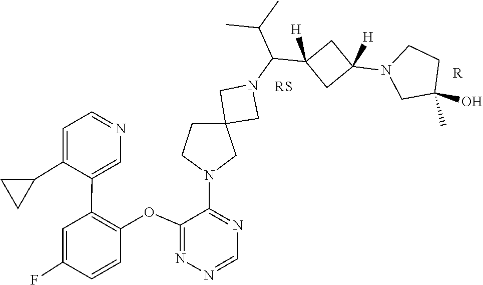 |
| | From intermediate 77 |
| Compound 138 | 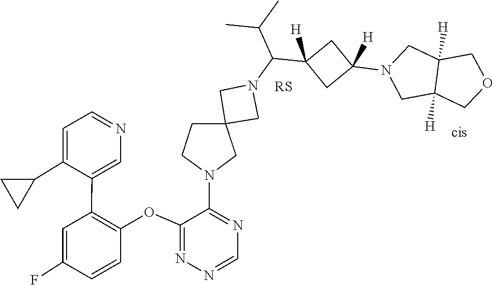 [SMILES: CC(C)C([C@H]1C[C@@H](N2C[C@H]3COC[C@H]3C2)C1)N1CC2(CCN(c3ncnnc3Oc3ccc(F)cc3-c3cnccc3C3CC3)C2)C1] |
| Compound 139 | 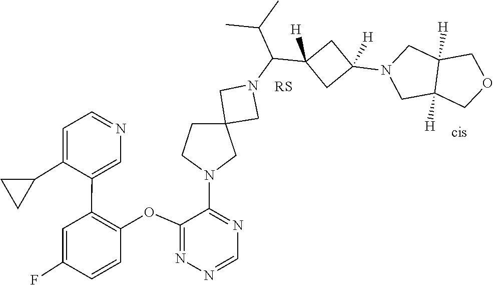 [SMILES: CC(C)C([C@H]1C[C@H](N2C[C@H]3COC[C@H]3C2)C1)N1CC2(CCN(c3ncnnc3Oc3ccc(F)cc3-c3cnccc3C3CC3)C2)C1] |
| | From intermediate 77 |
| Compound 140 | 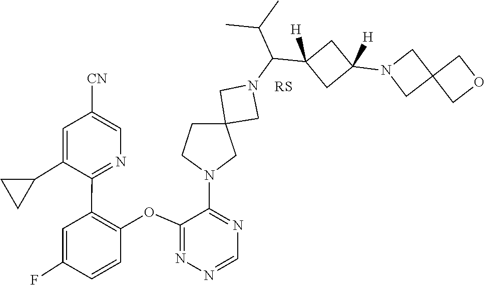 [SMILES: CC(C)C([C@H]1C[C@@H](N2CC3(COC3)C2)C1)N1CC2(CCN(c3ncnnc3Oc3ccc(F)cc3-c3ncc(C#N)cc3C3CC3)C2)C1] |
| Compound 141 | 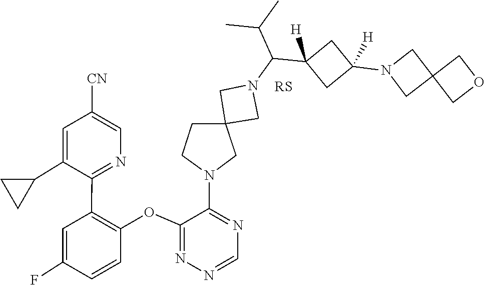 [SMILES: CC(C)C([C@H]1C[C@H](N2CC3(COC3)C2)C1)N1CC2(CCN(c3ncnnc3Oc3ccc(F)cc3-c3ncc(C#N)cc3C3CC3)C2)C1] |
| | from intermediate 82 |
| Compound 142 | 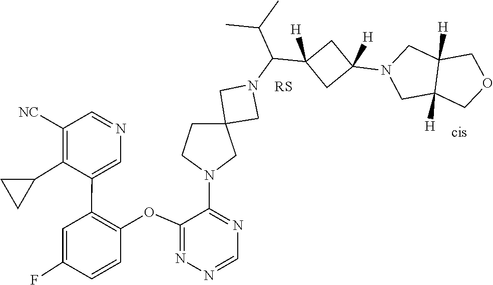 [SMILES: CC(C)C([C@H]1C[C@@H](N2C[C@H]3COC[C@H]3C2)C1)N1CC2(CCN(c3ncnnc3Oc3ccc(F)cc3-c3cncc(C#N)c3C3CC3)C2)C1] |
| Compound 143 | 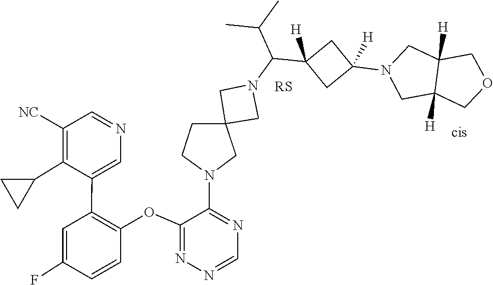 [SMILES: CC(C)C([C@H]1C[C@H](N2C[C@H]3COC[C@H]3C2)C1)N1CC2(CCN(c3ncnnc3Oc3ccc(F)cc3-c3cncc(C#N)c3C3CC3)C2)C1] |
| | from intermediate 86 |
| Compound 144 | 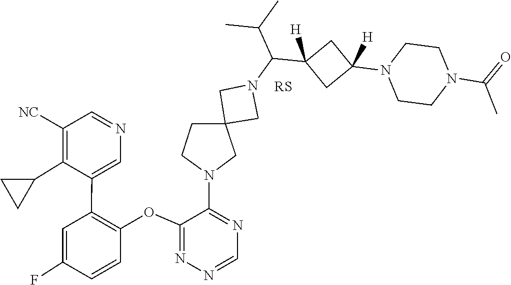 [SMILES: CC(=O)N1CCN([C@H]2C[C@@H](C(C(C)C)N3CC4(CCN(c5ncnnc5Oc5ccc(F)cc5-c5cncc(C#N)c5C5CC5)C4)C3)C2)CC1] |
| | from intermediate 86 |

### Preparation of Compound 331 and Compound 332

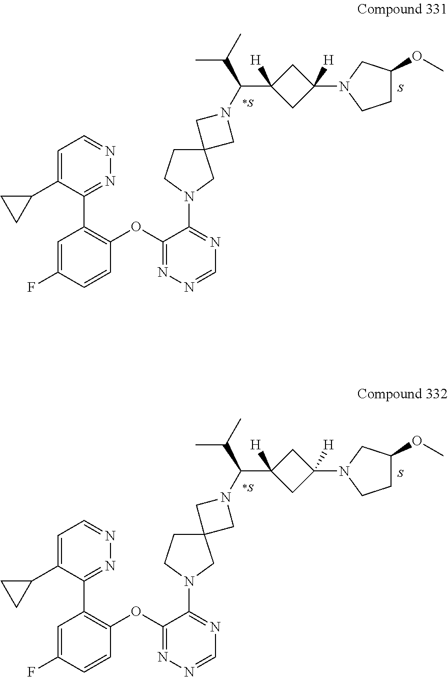

NaBH₃CN (433 mg, 6.89 mmol) was added to a solution consisting of intermediate 249a (800 mg, 1.38 mmol) and (S)-3-methoxypyrrolidine hydrochloride (418 mg, 4.13 mmol), MeOH (10 mL) and AcOH (0.237 mL). The mixture was stirred at 60° C. for 12 h. After cooling to RT, the mixture was adjusted to pH=8 with NH₃H₂O and purified by preparative HPLC using a Phenomenex Gemini 150 mm×25 mm×10 μm column (eluent: 30% to 60% (v/v) ACN and H₂O with 0.05% NH₃H₂O). The desired fractions were collected and lyophilized to afford Compound 331 (321 mg, 36% yield) and Compound 332 (49 mg, 5% yield) as a white solid.

### Compound 331

¹H NMR Methanol-d₄ (Varian_400 MHz): δ 9.01-8.85 (m, 1H), 8.29 (s, 1H), 7.63-7.47 (m, 1H), 7.45-7.28 (m, 2H), 7.21-7.07 (m, 1H), 4.02-3.90 (m, 1H), 3.88-3.68 (m, 2H), 3.64-3.43 (m, 2H), 3.29-3.16 (m, 7H), 2.81-2.70 (m, 1H), 2.70-2.60 (m, 2H), 2.60-2.53 (m, 1H), 2.49-2.39 (m, 1H), 2.26-2.13 (m, 3H), 2.11-1.96 (m, 4H), 1.86-1.66 (m, 5H), 1.08-0.98 (m, 2H), 0.97-0.86 (m, 6H), 0.83-0.75 (m, 2H).

### Compound 332

¹H NMR Methanol-d₄ (Varian_400 MHz): δ 9.00-8.87 (m, 1H), 8.30 (s, 1H), 7.60-7.48 (m, 1H), 7.45-7.32 (m, 2H), 7.19-7.10 (m, 1H), 4.03-3.92 (m, 1H), 3.85-3.69 (m, 2H), 3.67-3.57 (m, 1H), 3.56-3.45 (m, 1H), 3.30-3.14 (m, 7H), 2.95-2.86 (m, 1H), 2.81-2.53 (m, 4H), 2.32-2.16 (m, 2H), 2.15-1.97 (m, 6H), 1.89-1.66 (m, 4H), 1.08-0.98 (m, 2H), 0.96-0.85 (m, 6H), 0.84-0.74 (m, 2H).

The compounds reported below were prepared following an analogous methodology as described for Compound 331 and Compound 332 starting from the intermediate 249a (*S):

| Compound number | Structure |
|---|---|
| Compound 250 | 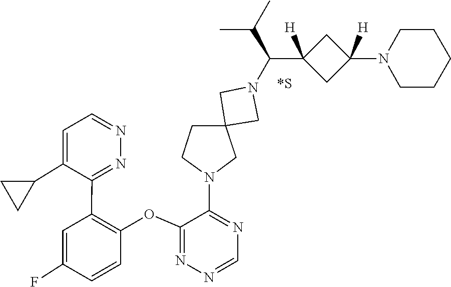 [SMILES: CC(C)[C@@H]([C@H]1C[C@@H](N2CCCCC2)C1)N1CC2(CCN(c3ncnnc3Oc3ccc(F)cc3-c3nnccc3C3CC3)C2)C1] |
| Compound 251 | 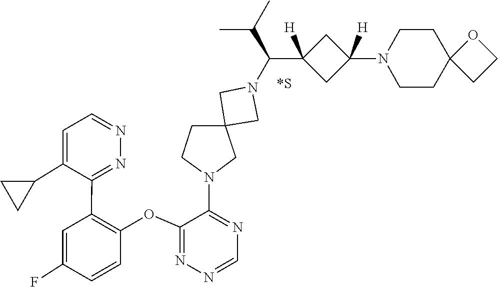 [SMILES: CC(C)[C@@H]([C@H]1C[C@@H](N2CCC3(CCO3)CC2)C1)N1CC2(CCN(c3ncnnc3Oc3ccc(F)cc3-c3nnccc3C3CC3)C2)C1] |
| Compound 253 | 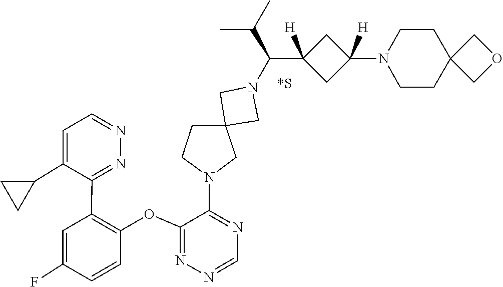 [SMILES: CC(C)[C@@H]([C@H]1C[C@@H](N2CCC3(CC2)COC3)C1)N1CC2(CCN(c3ncnnc3Oc3ccc(F)cc3-c3nnccc3C3CC3)C2)C1] |
| Compound 254 | 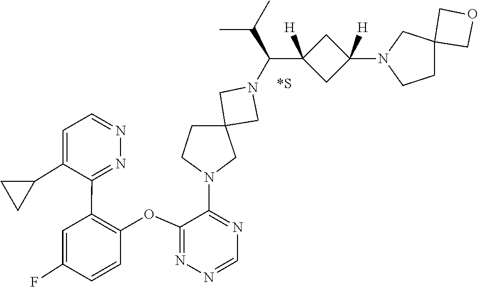 [SMILES: CC(C)[C@@H]([C@H]1C[C@@H](N2CCC3(COC3)C2)C1)N1CC2(CCN(c3ncnnc3Oc3ccc(F)cc3-c3nnccc3C3CC3)C2)C1] |
| Compound 256 | 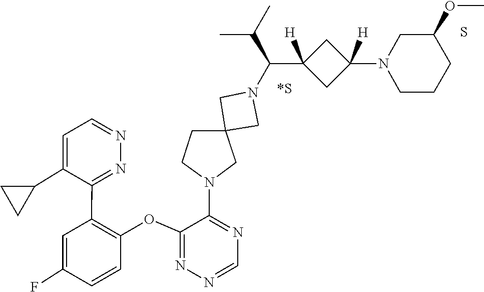 |
| Compound 257 | 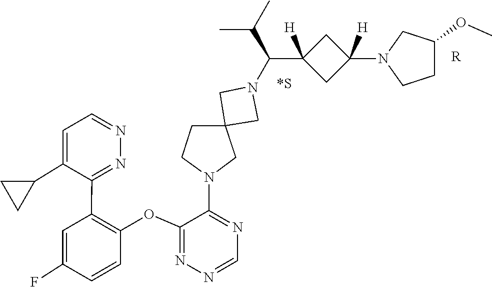 |
| Compound 258 | 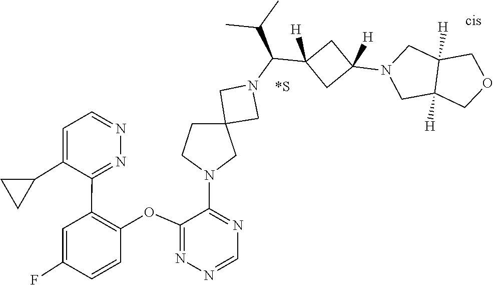 [SMILES: CC(C)[C@@H]([C@H]1C[C@@H](N2C[C@H]3COC[C@H]3C2)C1)N1CC2(CCN(c3ncnnc3Oc3ccc(F)cc3-c3nnccc3C3CC3)C2)C1] |
| Compound 259 | 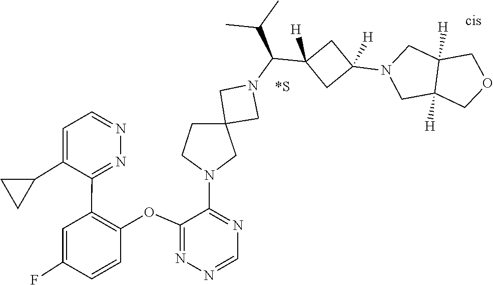 [SMILES: CC(C)[C@@H]([C@H]1C[C@H](N2C[C@H]3COC[C@H]3C2)C1)N1CC2(CCN(c3ncnnc3Oc3ccc(F)cc3-c3nnccc3C3CC3)C2)C1] |
| Compound 260 | 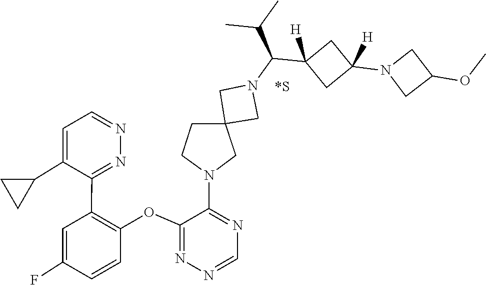 [SMILES: COC1CN([C@H]2C[C@@H]([C@H](C(C)C)N3CC4(CCN(c5ncnnc5Oc5ccc(F)cc5-c5nnccc5C5CC5)C4)C3)C2)C1] |
| Compound 261a | 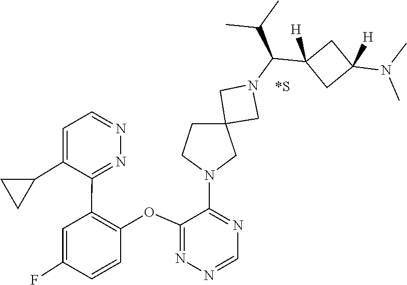 [SMILES: CC(C)[C@@H]([C@H]1C[C@@H](N(C)C)C1)N1CC2(CCN(c3ncnnc3Oc3ccc(F)cc3-c3nnccc3C3CC3)C2)C1] |
| Compound 261b | 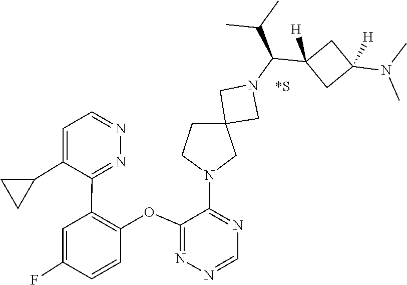 [SMILES: CC(C)[C@@H]([C@H]1C[C@H](N(C)C)C1)N1CC2(CCN(c3ncnnc3Oc3ccc(F)cc3-c3nnccc3C3CC3)C2)C1] |
| Compound 262 | 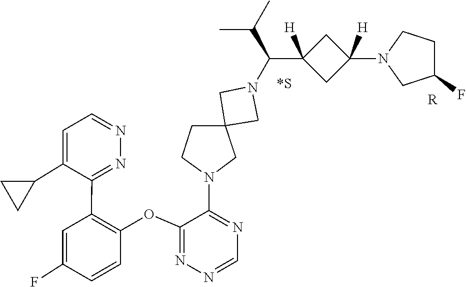 |
| Compound 263 | 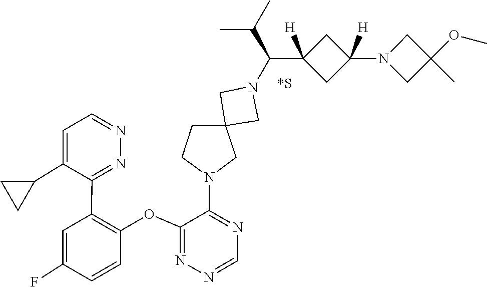 [SMILES: COC1(C)CN([C@H]2C[C@@H]([C@H](C(C)C)N3CC4(CCN(c5ncnnc5Oc5ccc(F)cc5-c5nnccc5C5CC5)C4)C3)C2)C1] |
| Compound 264 | 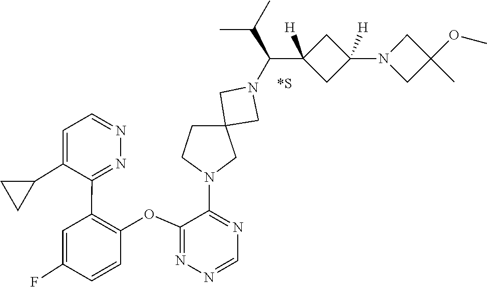 [SMILES: COC1(C)CN([C@H]2C[C@H]([C@H](C(C)C)N3CC4(CCN(c5ncnnc5Oc5ccc(F)cc5-c5nnccc5C5CC5)C4)C3)C2)C1] |
| Compound 265 | 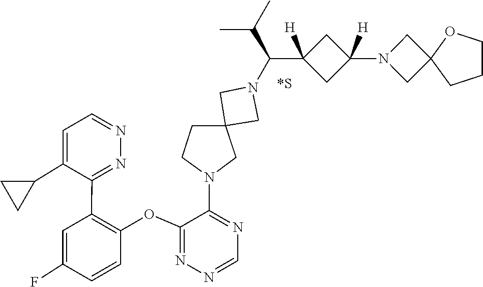 [SMILES: CC(C)[C@@H]([C@H]1C[C@@H](N2CC3(CCCO3)C2)C1)N1CC2(CCN(c3ncnnc3Oc3ccc(F)cc3-c3nnccc3C3CC3)C2)C1] |
| Compound 266 | 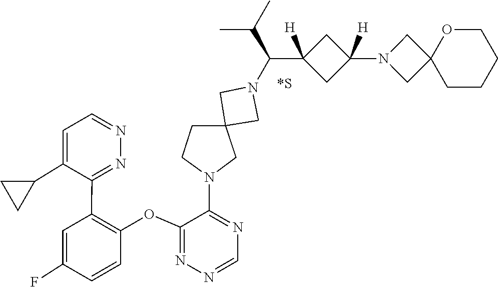 [SMILES: CC(C)[C@@H]([C@H]1C[C@@H](N2CC3(CCCCO3)C2)C1)N1CC2(CCN(c3ncnnc3Oc3ccc(F)cc3-c3nnccc3C3CC3)C2)C1] |
| Compound 268 | 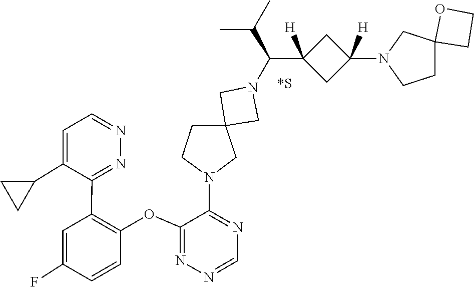 [SMILES: CC(C)[C@@H]([C@H]1C[C@@H](N2CCC3(CCO3)C2)C1)N1CC2(CCN(c3ncnnc3Oc3ccc(F)cc3-c3nnccc3C3CC3)C2)C1] |
| Compound 270 | 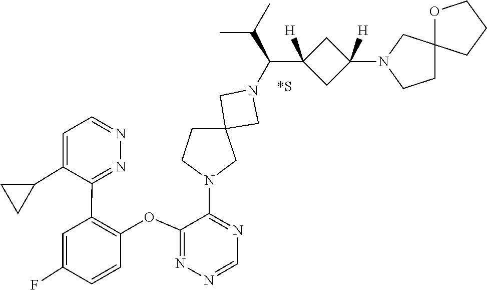 [SMILES: CC(C)[C@@H]([C@H]1C[C@@H](N2CCC3(CCCO3)C2)C1)N1CC2(CCN(c3ncnnc3Oc3ccc(F)cc3-c3nnccc3C3CC3)C2)C1] |
| Compound 272 | 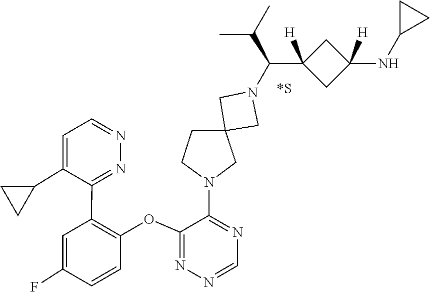 [SMILES: CC(C)[C@@H]([C@H]1C[C@@H](NC2CC2)C1)N1CC2(CCN(c3ncnnc3Oc3ccc(F)cc3-c3nnccc3C3CC3)C2)C1] |
| Compound 273 | 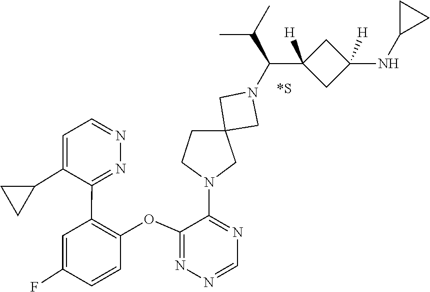 [SMILES: CC(C)[C@@H]([C@H]1C[C@H](NC2CC2)C1)N1CC2(CCN(c3ncnnc3Oc3ccc(F)cc3-c3nnccc3C3CC3)C2)C1] |
| Compound 274 | 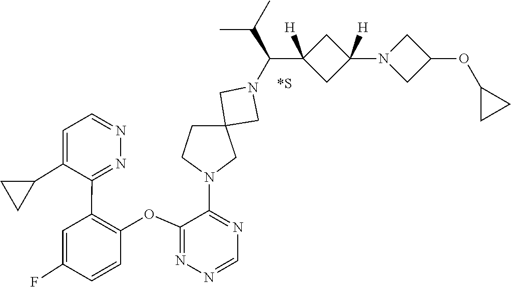 [SMILES: CC(C)[C@@H]([C@H]1C[C@@H](N2CC(OC3CC3)C2)C1)N1CC2(CCN(c3ncnnc3Oc3ccc(F)cc3-c3nnccc3C3CC3)C2)C1] |
| Compound 275 | 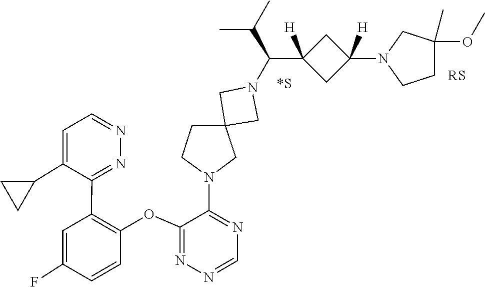 [SMILES: COC1(C)CCN([C@H]2C[C@@H]([C@H](C(C)C)N3CC4(CCN(c5ncnnc5Oc5ccc(F)cc5-c5nnccc5C5CC5)C4)C3)C2)C1] |
| Compound 276 | 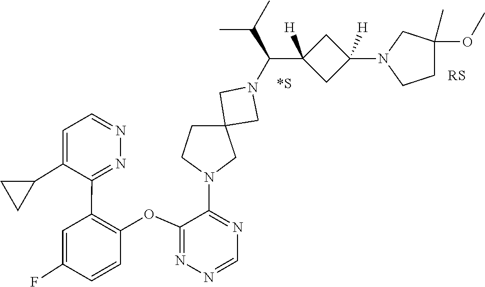 [SMILES: COC1(C)CCN([C@H]2C[C@H]([C@H](C(C)C)N3CC4(CCN(c5ncnnc5Oc5ccc(F)cc5-c5nnccc5C5CC5)C4)C3)C2)C1] |
| Compound 277 | 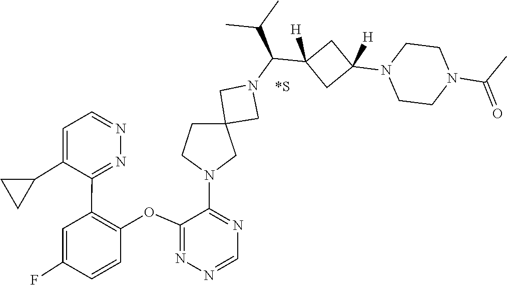 [SMILES: CC(=O)N1CCN([C@H]2C[C@@H]([C@H](C(C)C)N3CC4(CCN(c5ncnnc5Oc5ccc(F)cc5-c5nnccc5C5CC5)C4)C3)C2)CC1] |

The compounds reported below were prepared following an analogous methodology as described for Compound 331 starting from the intermediate 249b (*R):

| Compound number | Structure |
|---|---|
| Compound 252 | 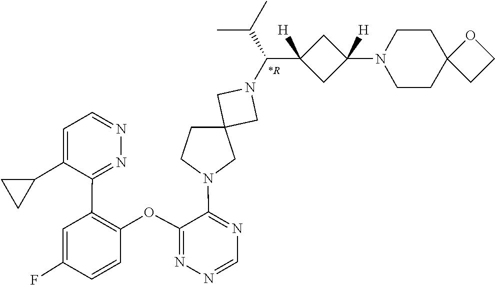 [SMILES: CC(C)[C@H]([C@H]1C[C@@H](N2CCC3(CCO3)CC2)C1)N1CC2(CCN(c3ncnnc3Oc3ccc(F)cc3-c3nnccc3C3CC3)C2)C1] |
| Compound 255 | 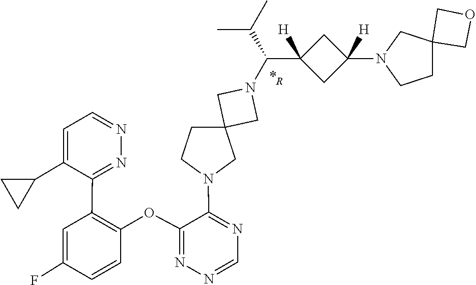 [SMILES: CC(C)[C@H]([C@H]1C[C@@H](N2CCC3(COC3)C2)C1)N1CC2(CCN(c3ncnnc3Oc3ccc(F)cc3-c3nnccc3C3CC3)C2)C1] |
| Compound 267 | 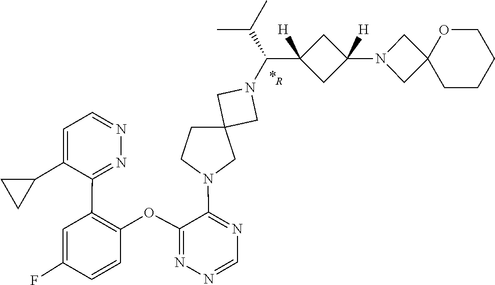 [SMILES: CC(C)[C@H]([C@H]1C[C@@H](N2CC3(CCCCO3)C2)C1)N1CC2(CCN(c3ncnnc3Oc3ccc(F)cc3-c3nnccc3C3CC3)C2)C1] |
| Compound 269 | 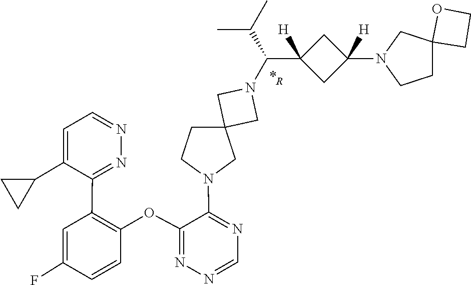 [SMILES: CC(C)[C@H]([C@H]1C[C@@H](N2CCC3(CCO3)C2)C1)N1CC2(CCN(c3ncnnc3Oc3ccc(F)cc3-c3nnccc3C3CC3)C2)C1] |
| Compound 271 | 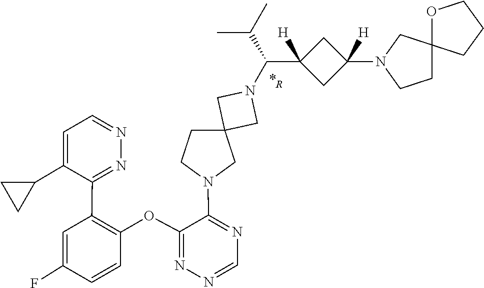 [SMILES: CC(C)[C@H]([C@H]1C[C@@H](N2CCC3(CCCO3)C2)C1)N1CC2(CCN(c3ncnnc3Oc3ccc(F)cc3-c3nnccc3C3CC3)C2)C1] |
| Compound 328 | 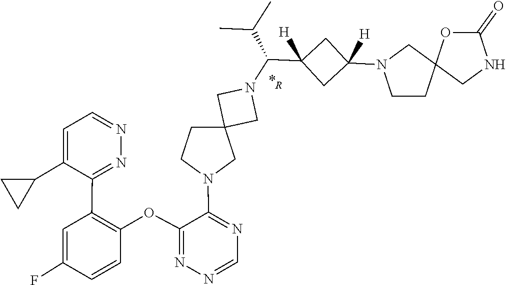 [SMILES: CC(C)[C@H]([C@H]1C[C@@H](N2CCC3(CNC(=O)O3)C2)C1)N1CC2(CCN(c3ncnnc3Oc3ccc(F)cc3-c3nnccc3C3CC3)C2)C1] |
| | from intermediate 246 |

### Preparation of Compound 9

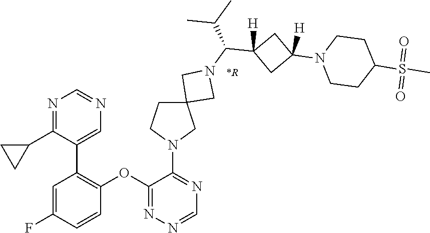 [SMILES: CC(C)[C@H]([C@H]1C[C@@H](N2CCC(S(C)(=O)=O)CC2)C1)N1CC2(CCN(c3ncnnc3Oc3ccc(F)cc3-c3cncnc3C3CC3)C2)C1]

NaBH₃CN (162 mg; 2.58 mmol) was added to a mixture of intermediate 38b (*R) (700 mg; 1.288 mmol), 4-(methylsulfonyl)piperidine (1.05 g; 6.438 mmol), AcOH (74 μL; 1.3 mmol) in MeOH (41 mL). Then, the reaction mixture was heated at 60° C. for 24 h. The reaction mixture was cooled to r.t, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.7% NH₄OH, 7% MeOH, 93% DCM). The pure fractions were collected and evaporated to dryness. The residue (684 mg) was freeze-dried with water-ACN to give 655 mg (73%) of final compound 9.

¹H NMR (500 MHz, DMSO-d₆) δ ppm 8.95 (br, 1H) 8.41 (br s, 2H) 7.57 (br d, J=3.4 Hz, 1H) 7.41-7.49 (m, 2H) 3.39-3.79 (m, 4H) 2.85-3.15 (m, 10H) 2.38-2.47 (m, 1H) 2.02-2.16 (m, 2H) 1.96 (br d, J=9.8 Hz, 5H) 1.80-1.89 (m, 1H) 1.41-1.78 (m, 8H) 1.00 (m, 3H) 0.82 (t, J=6.2 Hz, 7H)

### Preparation of Compound 10 and Compound 33

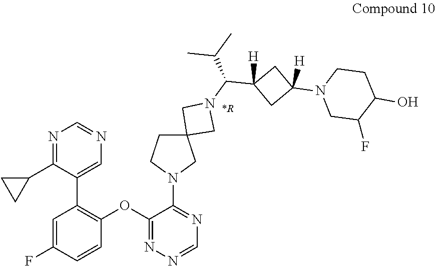 [SMILES: CC(C)[C@H]([C@H]1C[C@@H](N2CCC(O)C(F)C2)C1)N1CC2(CCN(c3ncnnc3Oc3ccc(F)cc3-c3cncnc3C3CC3)C2)C1]

piperidine moiety is one cis isomer (undetermined which cis)

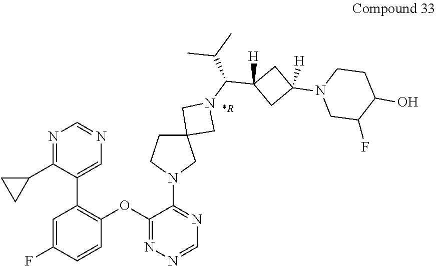 [SMILES: CC(C)[C@H]([C@H]1C[C@H](N2CCC(O)C(F)C2)C1)N1CC2(CCN(c3ncnnc3Oc3ccc(F)cc3-c3cncnc3C3CC3)C2)C1]

piperidine moiety is one cis isomer (undetermined which cis)

NaBH₃CN (40.7 mg; 0.65 mmol) was added to a mixture of intermediate 38b (*R) (176.1 mg; 0.33 mmol), cis-3-fluoropiperidin-4-ol (201.6 mg; 1.30 mmol), AcOH (19 μL; 0.32 mmol) in MeOH (15 mL). Then, the reaction mixture was heated at 60° C. for 18 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.1% NH₄OH, 12% MeOH, 88% DCM). The pure fractions were collected and evaporated to dryness. The residue (650 mg) was purified by achiral SFC (CHIRALPAK AD-H 5 μm 250*30 mm, mobile phase: 80% CO₂, 20% EtOH (0.3% iPrNH₂)). The pure fractions were collected and the solvent was evaporated. After freeze-drying with a mixture of water-ACN, 29 mg (14%) of Compound 10 and 25 mg (12%) of Compound 33.

Compound 10: ¹H NMR (500 MHz, DMSO-d₆) δ ppm 8.88-8.97 (m, 1H), 8.41 (br s, 2H), 7.57 (br s, 1H), 7.47 (br d, J=7.5 Hz, 2H), 4.89 (d, J=4.7 Hz, 1H), 4.43-4.60 (m, 1H), 3.38-3.79 (m, 6H), 2.87-3.14 (m, 5H), 1.39-2.32 (m, 15H), 1.00 (br d, J=6.9 Hz, 3H), 0.83 (br t, J=5.8 Hz, 7H)

### Preparation of Compound 12 and Compound 13

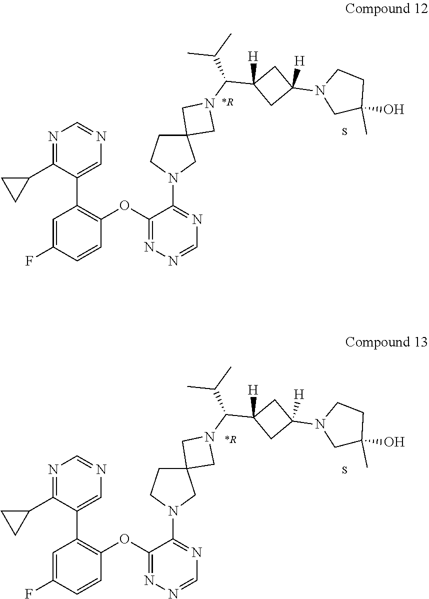

NaBH₃CN (35 mg; 0.55 mmol) was added to a mixture of intermediate 38b (*R) (150 mg; 0.28 mmol), (3S)-3-methylpyrrolidin-3-ol (140 mg; 1.38 mmol), AcOH (16 μL; 0.28 mmol) in MeOH (9 mL). Then, the reaction mixture was heated at 60° C. for 18 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0.3% NH₄OH, 3% MeOH, 97% DCM to 1% NH₄OH, 10% MeOH, 90% DCM). The pure fractions were collected and evaporated to give 130 mg of a mixture of compound 12 and compound 13. The residue (130 mg) was purified by reverse phase (mobile phase: gradient from 65% NH₄CO₃ (0.2%), 35% ACN to 25% NH₄CO₃ (0.2%), 75% ACN). The pure fractions were collected and evaporated to dryness which was freeze-dried with water-ACN to give 80 mg (46%) of final compound 12 as a white solid.

### Compound 12

¹H NMR (400 MHz, DMSO-d₆) δ ppm 8.88-8.96 (m, 1H), 8.41 (br s, 2H), 7.57 (br d, J=2.0 Hz, 1H), 7.41-7.51 (m, 2H), 4.45 (s, 1H), 3.38-3.77 (m, 4H), 2.88-3.20 (m, 4H), 2.58-2.64 (m, 1H), 2.36-2.44 (m, 1H), 2.32 (s, 3H), 1.90-2.08 (m, 4H), 1.46-1.90 (m, 8H), 1.21 (s, 3H), 0.90-1.07 (m, 3H), 0.82 (dd, J=6.8, 3.1 Hz, 7H)

### Preparation of Compound 14 and Compound 15

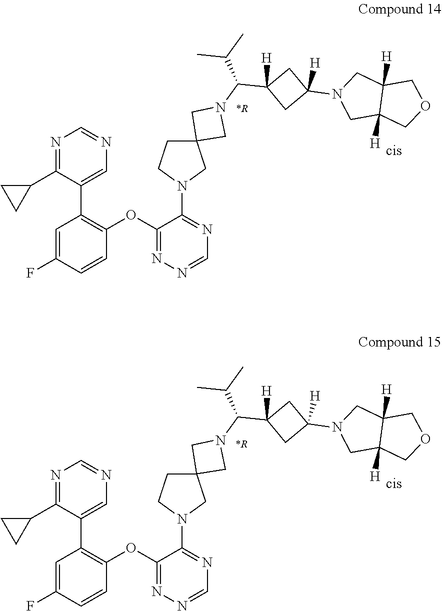 [SMILES: CC(C)[C@H]([C@H]1C[C@@H](N2C[C@H]3COC[C@H]3C2)C1)N1CC2(CCN(c3ncnnc3Oc3ccc(F)cc3-c3cncnc3C3CC3)C2)C1 ; CC(C)[C@H]([C@H]1C[C@H](N2C[C@H]3COC[C@H]3C2)C1)N1CC2(CCN(c3ncnnc3Oc3ccc(F)cc3-c3cncnc3C3CC3)C2)C1]

NaBH₃CN (35 mg; 0.55 mmol) was added to a mixture of intermediate 38b (*R) (150 mg; 0.28 mmol), hexahydro-1H-furo[3,4-C]pyrrole (156 mg; 1.38 mmol), AcOH (16 μL; 0.28 mmol) in MeOH (9 mL). Then, the reaction mixture was heated at 60° C. for 48 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0.3% NH₄OH, 3% MeOH, 97% DCM to 0.7% NH₄OH, 7% MeOH, 93% DCM). The pure fractions were collected and evaporated to give 79 mg which were freeze-dried with water-ACN to give 79 mg (44%) of compound 14 as a white solid and 95 mg of a mixture of compound 14 and compound 15 which was not purified further.

### Compound 14

¹H NMR (500 MHz, DMSO-d₆) δ ppm 8.88-8.98 (m, 1H), 8.36-8.46 (m, 2H), 7.56 (br d, J=4.1 Hz, 1H), 7.41-7.50 (m, 2H), 3.66-3.76 (m, 3H), 3.39-3.63 (m, 4H), 3.35 (br dd, J=8.5, 3.7 Hz, 3H), 2.88-3.16 (m, 4H), 2.62-2.68 (m, 2H), 2.53-2.61 (m, 2H), 2.34-2.48 (m, 4H), 2.14-2.22 (m, 2H), 1.48-2.11 (m, 12H), 0.91-1.05 (m, 3H), 0.82 (dd, J=6.9, 4.9 Hz, 7H)

### Preparation of Compound 16 and Compound 17

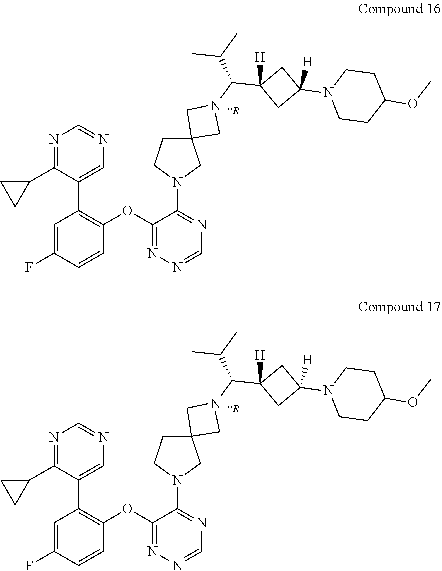 [SMILES: COC1CCN([C@H]2C[C@@H]([C@@H](C(C)C)N3CC4(CCN(c5ncnnc5Oc5ccc(F)cc5-c5cncnc5C5CC5)C4)C3)C2)CC1 ; COC1CCN([C@H]2C[C@H]([C@@H](C(C)C)N3CC4(CCN(c5ncnnc5Oc5ccc(F)cc5-c5cncnc5C5CC5)C4)C3)C2)CC1]

NaBH₃CN (139 mg; 2.21 mmol) was added to a mixture of intermediate 38b (*R) (600 mg; 1.1 mmol), 4-methoxypiperidine (636 mg; 5.52 mmol), AcOH (64 μL; 1.1 mmol) in MeOH (15 mL). Then, the reaction mixture was heated at 60° C. for 18 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.1% NH₄OH, 6% MeOH, 94% DCM). The pure fractions were collected and evaporated to dryness to give 355 mg of compound 16 and 155 mg mixture of compound 16 and compound 17. The compound 16 (355 mg) was further purified by reverse phase chromatography (mobile phase: gradient from 40% NH₄CO₃ (0.2%), 60% ACN to 10% NH₄CO₃ (0.2%), 90% ACN). The pure fractions were collected and evaporated to dryness to give 264 mg of compound 16 which was freeze-dried with water-ACN to give 250 mg (35%) of final compound as a white solid.

### Compound 16

¹H NMR (400 MHz, DMSO-d₆) δ ppm 8.88-8.98 (m, 1H), 8.41 (br s, 2H), 7.57 (br s, 1H), 7.40-7.50 (m, 2H), 3.39-3.79 (m, 4H), 3.20 (s, 3H), 2.88-3.16 (m, 6H), 2.36-2.43 (m, 1H), 2.00-2.18 (m, 2H), 1.70-2.00 (m, 10H), 1.55-1.67 (m, 2H), 1.46 (q, J=9.7 Hz, 1H), 1.29-1.40 (m, 2H), 1.00 (br s, 3H), 0.82 (br dd, J=6.9, 3.9 Hz, 7H)

### Preparation of Compound 18

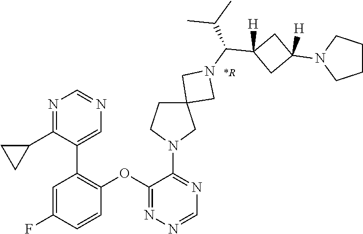 [SMILES: CC(C)[C@H]([C@H]1C[C@@H](N2CCCC2)C1)N1CC2(CCN(c3ncnnc3Oc3ccc(F)cc3-c3cncnc3C3CC3)C2)C1]

Compound 18 was prepared by an analogous procedure as was used for the synthesis of compound 12 and compound 13 starting from intermediate 38b (*R) and pyrrolidine. 40 mg (36%) of compound 18 was obtained.

### Preparation of Compound 20 and Compound 21

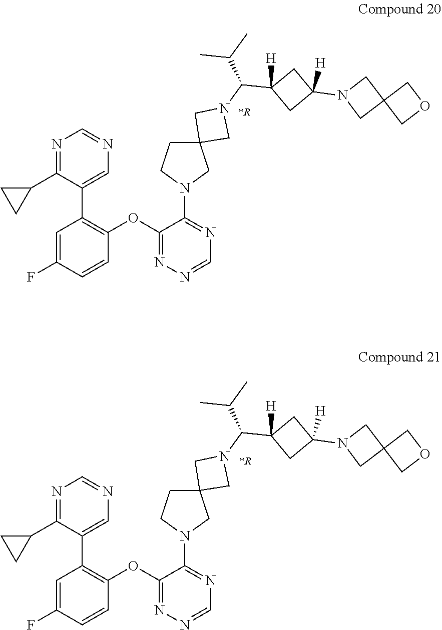 [SMILES: CC(C)[C@H]([C@H]1C[C@@H](N2CC3(COC3)C2)C1)N1CC2(CCN(c3ncnnc3Oc3ccc(F)cc3-c3cncnc3C3CC3)C2)C1 ; CC(C)[C@H]([C@H]1C[C@H](N2CC3(COC3)C2)C1)N1CC2(CCN(c3ncnnc3Oc3ccc(F)cc3-c3cncnc3C3CC3)C2)C1]

Compound 20 and Compound 21 were prepared by an analogous procedure as was used for the synthesis of Compound 7 and Compound 8 starting from intermediate 38b (*R) and 2-oxa-6-azaspiro[3.3]heptane. 45 mg (26%) of compound 20 and 45 mg (26%) of compound 21 were obtained.

### Compound 20

¹H NMR (500 MHz, DMSO-d₆) δ ppm 8.93 (br s, 1H), 8.41 (br s, 2H), 7.57 (br s, 1H), 7.37-7.49 (m, 2H), 4.56 (s, 4H), 3.39-3.82 (m, 4H), 3.13-3.23 (m, 4H), 2.85-3.12 (m, 4H), 2.71-2.83 (m, 1H), 1.84-2.07 (m, 5H), 1.68-1.83 (m, 2H), 1.51-1.67 (m, 2H), 1.42 (q, J=9.8 Hz, 1H), 0.62-1.09 (m, 10H)

### Preparation of Compound 22

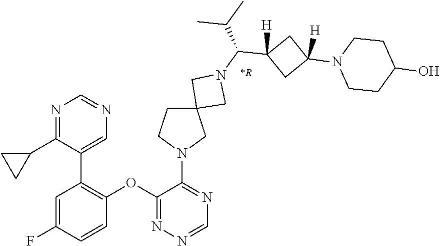 [SMILES: CC(C)[C@H]([C@H]1C[C@@H](N2CCC(O)CC2)C1)N1CC2(CCN(c3ncnnc3Oc3ccc(F)cc3-c3cncnc3C3CC3)C2)C1]

TBAF (11.5 mL; 11.3 mmol; 1 M in THF) was added to a solution of intermediate 39 (1.09 g; 1.26 mmol) in MeTHF (25 mL) and the reaction was stirred at RT for 24 h. The mixture was poured onto a 10% aqueous solution of K₂CO₃. The organic layer was extracted with EtOAc (3×), dried over MgSO₄, filtered and evaporated till dryness. The residue (1.36 g) was purified by chromatography over silica gel (mobile phase: gradient from 0% NH₄OH, 0% MeOH, 100% DCM to 0.7% NH₄OH, 7% MeOH, 93% DCM). The pure fractions were collected and evaporated to dryness. The residue (604 mg) was purified by reverse phase (mobile phase: gradient from 75% NH₄CO₃ (0.2%), 25% ACN to 35% NH₄CO₃ (0.2%), 65% ACN).

The pure fractions were collected and evaporated to dryness which was freeze-dried with water-ACN to give 313 mg (40%) of compound 22 as a white solid.

### Preparation of Compound 313 and Compound 314

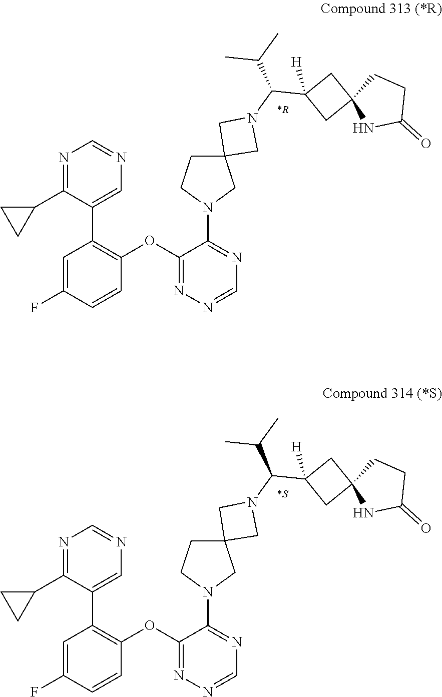 [SMILES: CC(C)[C@H]([C@H]1C[C@]2(CCC(=O)N2)C1)N1CC2(CCN(c3ncnnc3Oc3ccc(F)cc3-c3cncnc3C3CC3)C2)C1 ; CC(C)[C@@H]([C@H]1C[C@]2(CCC(=O)N2)C1)N1CC2(CCN(c3ncnnc3Oc3ccc(F)cc3-c3cncnc3C3CC3)C2)C1]

ZnCl₂ (450 mg, 3.30 mmol) was added to a solution of intermediate 236 (200 mg, 1.02 mmol) and intermediate 33 (450 mg, 1.07 mmol) in MeOH (10 mL). The mixture was stirred at 70° C. for 2 h. Then, NaBH₃CN (200 mg, 3.18 mmol) was added. The resulting mixture was stirred at 70° C. for additional 2 h. After cooling to RT, the mixture was quenched with water and filtered. The filtrate was evaporated to give a crude product, which was first purified by preparative HPLC (column: Phenomenex Gemini NX-C18 75*30 mm*3 um, mobile phase A: H₂O (0.05% NH₃H₂O+10 mM NH₄HCO₃), mobile phase B: ACN, gradient condition from 35% B to 65% B). The pure fractions were collected and lyophilized to give a 100 mg residue which was further separated by SFC (DAICEL CHIRALPAK AD-H (250 mm*30 mm, 10 um); mobile phase: A: Supercritical CO₂, B: 0.1% NH₃H₂O EtOH, A:B=50:50 at 80 mL/min). The desired fractions were collected and the volatiles were removed in vacuo. The residue was re-suspended in water (10 mL) and lyophilized to afford Compound 313 (23 mg, 4% yield) and Compound 314 (30 mg, 5% yield) as a white powder.

The compounds reported below were prepared following an analogous methodology as described for Compound 313 and Compound 314 starting from the appropriate intermediates:

| Compound Number | Structure |
|---|---|
| Compound 319 | 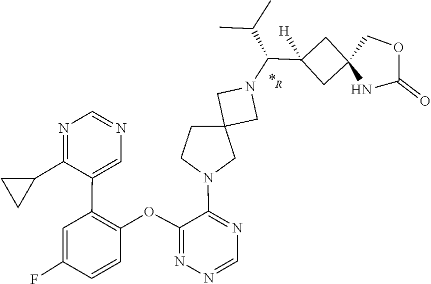 [SMILES: CC(C)[C@H]([C@H]1C[C@]2(COC(=O)N2)C1)N1CC2(CCN(c3ncnnc3Oc3ccc(F)cc3-c3cncnc3C3CC3)C2)C1] |
| Compound 320 | 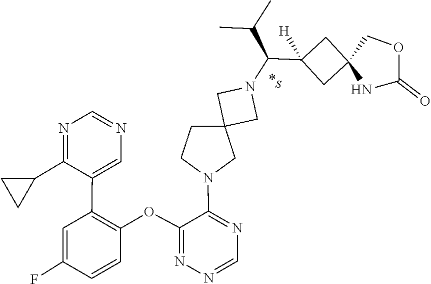 [SMILES: CC(C)[C@@H]([C@H]1C[C@]2(COC(=O)N2)C1)N1CC2(CCN(c3ncnnc3Oc3ccc(F)cc3-c3cncnc3C3CC3)C2)C1] |
| | from intermediate 33 and intermediate 245, SFC separation: |
| | DAICEL CHIRALPAK AS (250 mm x 30 mm, 10 um), |
| | mobile phase: A: Supercritical CO₂, B: 0.1% NH₃H₂O |
| | EtOH, eluent: isocratic 55% B (v/v) |
| Compound 321 | 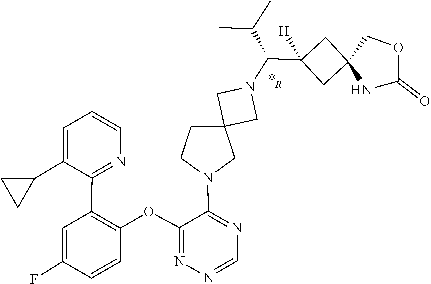 [SMILES: CC(C)[C@H]([C@H]1C[C@]2(COC(=O)N2)C1)N1CC2(CCN(c3ncnnc3Oc3ccc(F)cc3-c3ncccc3C3CC3)C2)C1] |
| Compound 322 | 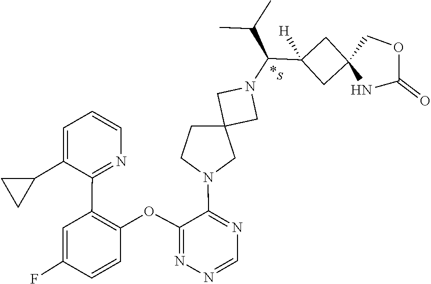 [SMILES: CC(C)[C@@H]([C@H]1C[C@]2(COC(=O)N2)C1)N1CC2(CCN(c3ncnnc3Oc3ccc(F)cc3-c3ncccc3C3CC3)C2)C1] |
| | from intermediate 50 and intermediate 245, SFC condition: |
| | SFC separation: DAICEL CHIRALPAK AS (250 mm x |
| | 30 mm, 10 um), mobile phase: A: Supercritical CO₂, B: 0.1% |
| | NH₃H₂O EtOH, eluent: isocratic 45% B (v/v) |
| Compound 323 | 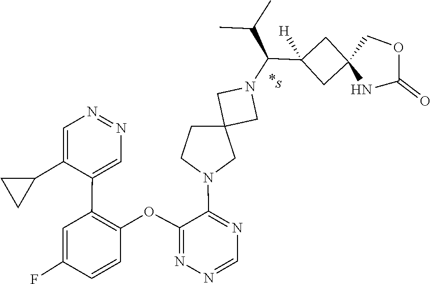 [SMILES: CC(C)[C@@H]([C@H]1C[C@]2(COC(=O)N2)C1)N1CC2(CCN(c3ncnnc3Oc3ccc(F)cc3-c3cnncc3C3CC3)C2)C1] |
| | from intermediate 296 and intermediate 245, SFC |
| | separation: Phenomenex Lux Cellulose-4 (250 x 30 mm, |
| | 5 um), mobile phase: A: Supercritical CO₂, B: 0.1% |
| | NH₃H₂O EtOH, eluent: A:B = 50:50 at 60 mL/min |
| Compound 324 | 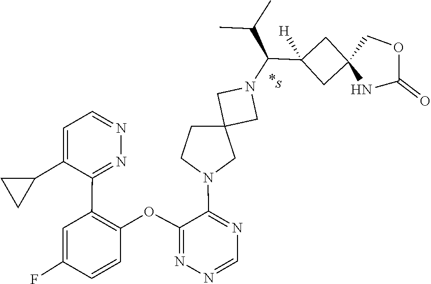 [SMILES: CC(C)[C@@H]([C@H]1C[C@]2(COC(=O)N2)C1)N1CC2(CCN(c3ncnnc3Oc3ccc(F)cc3-c3nnccc3C3CC3)C2)C1] |
| | from intermediate 297 and intermediate 245, SFC condition: |
| | DAICEL CHIRALPAK IG (250 mm x 30 mm, 10 um), |
| | mobile phase: A: Supercritical CO₂, B: 0.1% NH₃H₂O |
| | EtOH, eluent: A:B = 65:35 at 70 mL/min |

### Preparation of Compound 347

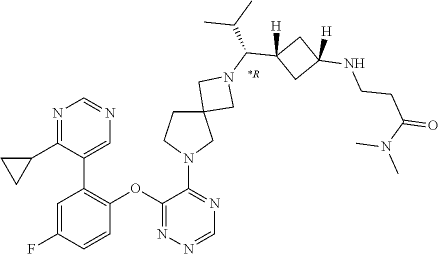 [SMILES: CC(C)[C@H]([C@H]1C[C@@H](NCCC(=O)N(C)C)C1)N1CC2(CCN(c3ncnnc3Oc3ccc(F)cc3-c3cncnc3C3CC3)C2)C1]

Compound 1a (150 mg, 0.275 mmol), N,N-dimethylacrylamide (55 mg, 0.56 mmol) and TEA (110 mg, 1.09 mmol) were added into a 10 mL sealed tube, followed with addition of EtOH (5 mL). The mixture was stirred at 70° C. for 12 h before it was cooled to RT. The reaction mixture was concentrated in vacuo to yield Compound 347 (200 mg, crude) as a yellow oil, which was used directly in next step without further purification.

The compound reported below was prepared following an analogous methodology as described for Compound 347 starting from Compound 1a:

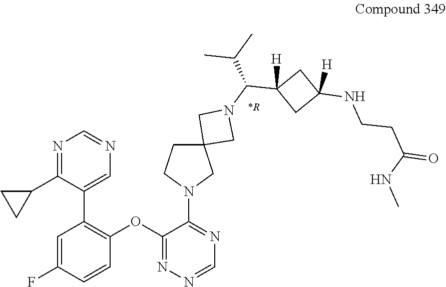 [SMILES: CNC(=O)CCN[C@H]1C[C@@H]([C@@H](C(C)C)N2CC3(CCN(c4ncnnc4Oc4ccc(F)cc4-c4cncnc4C4CC4)C3)C2)C1]

### Preparation of Compound 357

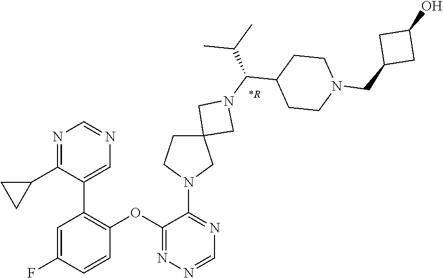 [SMILES: CC(C)[C@H](C1CCN(C[C@H]2C[C@@H](O)C2)CC1)N1CC2(CCN(c3ncnnc3Oc3ccc(F)cc3-c3cncnc3C3CC3)C2)C1]

Formic acid (0.2 mL, 5.3 mmol) was added drop-wise to a solution of intermediate 270 (350 mg, 0.462 mmol) in ACN (3 mL) and H₂O (1 mL). The resulting mixture was stirred at RT for 12 h. The reaction mixture was concentrated under reduced pressure and the crude product was purified by preparative HPLC (Welch Xtimate C18 150*30 mm*5 um, mobile phase A: water with 0.225% formic acid, mobile phase B: ACN, gradient condition: 8% B to 30% B v/v). The desired fractions were collected and lyophilized to afford Compound 357 (305 mg, 87% yield) as a white solid.

The compound reported below was prepared following an analogous methodology as described for Compound 357 starting from the corresponding intermediate:

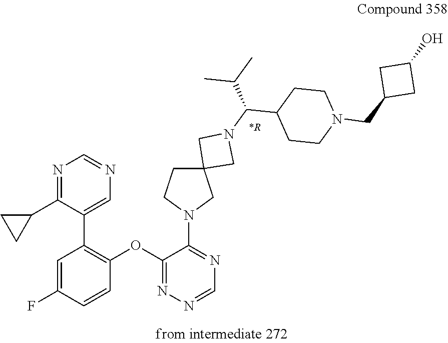 [SMILES: CC(C)[C@H](C1CCN(C[C@H]2C[C@H](O)C2)CC1)N1CC2(CCN(c3ncnnc3Oc3ccc(F)cc3-c3cncnc3C3CC3)C2)C1]

### Preparation of Compound 23

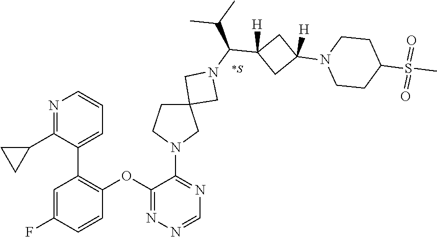 [SMILES: CC(C)[C@@H]([C@H]1C[C@@H](N2CCC(S(C)(=O)=O)CC2)C1)N1CC2(CCN(c3ncnnc3Oc3ccc(F)cc3-c3cccnc3C3CC3)C2)C1]

NaBH₃CN (46 mg; 0.74 mmol) was added to a mixture of intermediate 46b (*S) (200 mg; 0.37 mmol), 4-(methylsulfonyl)piperidine (301 mg; 1.84 mmol), AcOH (21 μL; 0.36 mmol) in MeOH (12 mL). Then, the reaction mixture was heated at 60° C. for 18 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0.3% NH₄OH, 3% MeOH, 97% DCM to 0.7% NH₄OH, 10% MeOH, 90% DCM). The pure fractions were collected and evaporated to dryness. The resulting residue was freeze-dried with water-ACN to give 145 mg (57%) of compound 23 as a white solid.

The compounds reported below were prepared following an analogous methodology as described for Compound 23 starting from intermediate 46b (*S):

| Compound Number | Structure |
|---|---|
| Compound 289 | 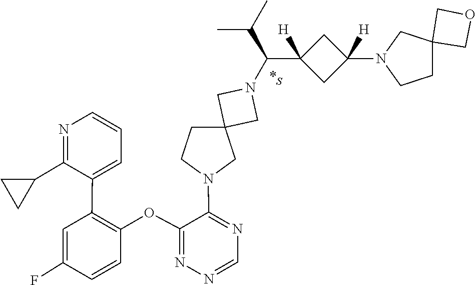 [SMILES: CC(C)[C@@H]([C@H]1C[C@@H](N2CCC3(COC3)C2)C1)N1CC2(CCN(c3ncnnc3Oc3ccc(F)cc3-c3cccnc3C3CC3)C2)C1] |
| Compound 290 | 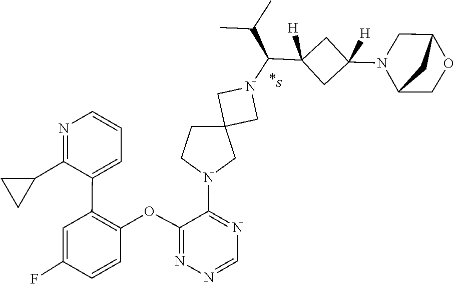 [SMILES: CC(C)[C@@H]([C@H]1C[C@@H](N2C[C@H]3C[C@@H]2CO3)C1)N1CC2(CCN(c3ncnnc3Oc3ccc(F)cc3-c3cccnc3C3CC3)C2)C1] |
| Compound 291 | 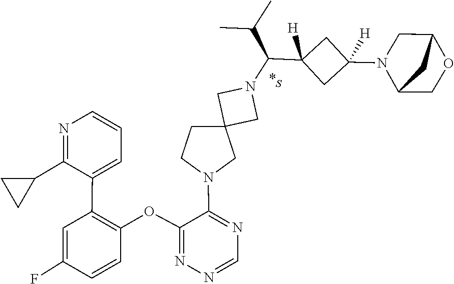 [SMILES: CC(C)[C@@H]([C@H]1C[C@H](N2C[C@H]3C[C@@H]2CO3)C1)N1CC2(CCN(c3ncnnc3Oc3ccc(F)cc3-c3cccnc3C3CC3)C2)C1] |

### Preparation of Compound 351

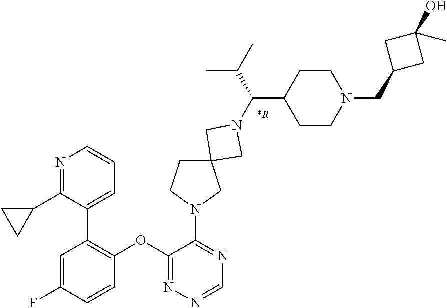 [SMILES: CC(C)[C@H](C1CCN(C[C@H]2C[C@@](C)(O)C2)CC1)N1CC2(CCN(c3ncnnc3Oc3ccc(F)cc3-c3cccnc3C3CC3)C2)C1]

To a solution of Compound 352 (40 mg, 0.072 mmol) in ACN (3 mL) were added intermediate 268 (35 mg, 0.180 mmol), K₂CO₃ (50 mg, 0.359 mmol) and KI (24 mg, 0.145 mmol). The resulting mixture was stirred at 70° C. for 24 h. The reaction mixture was cooled to RT and evaporated under reduced pressure. The resulting residue was partitioned between DCM (8 mL) and H₂O (5 mL). The aqueous layer was extracted with DCM (8 mL×2). The combined organic layers were washed with brine (5 mL), dried over anhydrous Na₂SO₄. After filtration and concentration, the crude product was purified by preparative HPLC (column: Phenomenex Gemini-NX 80 mm×40 mm 3 μm, mobile phase A: H₂O (0.05% NH₃H₂O+10 mM NH₄HCO₃), mobile phase B: ACN, flow rate: 30 mL/min, gradient condition from 40% B to 70% B). The desired fractions were collected and lyophilized to afford compound 351 (14 mg, 28% yield) as a white powder.

### Preparation of Compound 25

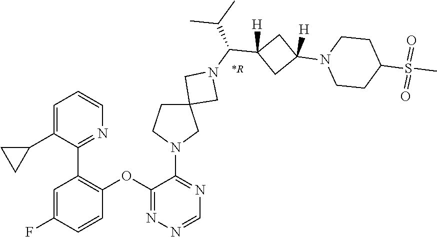 [SMILES: CC(C)[C@H]([C@H]1C[C@@H](N2CCC(S(C)(=O)=O)CC2)C1)N1CC2(CCN(c3ncnnc3Oc3ccc(F)cc3-c3ncccc3C3CC3)C2)C1]

NaBH₃CN (25 mg; 0.41 mmol) was added to a mixture of intermediate 52a (*R) (110 mg; 0.2 mmol), 4-(methylsulfonyl)piperidine (165 mg; 1.01 mmol), AcOH (12 μL; 0.2 mmol) in MeOH (8 mL). Then, the reaction mixture was heated at 60° C. for 24 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.1% NH₄OH, 5% MeOH, 95% DCM). The pure fractions were collected and evaporated to dryness. The residue (72 mg) was freeze-dried with water-ACN to give 65 mg (47%) of compound 25 as white solid.

The compounds reported below were prepared following an analogous methodology as the one reported for the preparation of compound 25, starting from the appropriate starting materials (e.g. intermediate 52a (*R) or any other relevant intermediates):

| Compound Number | Structure |
|---|---|
| Compound 147 | 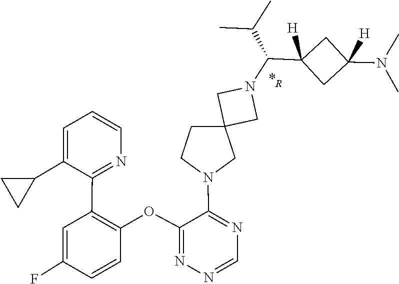 [SMILES: CC(C)[C@H]([C@H]1C[C@@H](N(C)C)C1)N1CC2(CCN(c3ncnnc3Oc3ccc(F)cc3-c3ncccc3C3CC3)C2)C1] |
| Compound 148 | 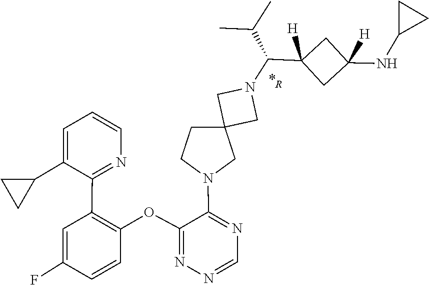 [SMILES: CC(C)[C@H]([C@H]1C[C@@H](NC2CC2)C1)N1CC2(CCN(c3ncnnc3Oc3ccc(F)cc3-c3ncccc3C3CC3)C2)C1] |
| Compound 149 | 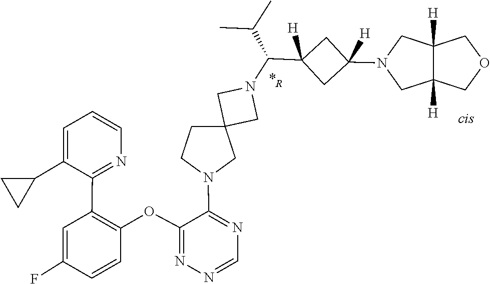 [SMILES: CC(C)[C@H]([C@H]1C[C@@H](N2C[C@H]3COC[C@H]3C2)C1)N1CC2(CCN(c3ncnnc3Oc3ccc(F)cc3-c3ncccc3C3CC3)C2)C1] |
| Compound 150 | 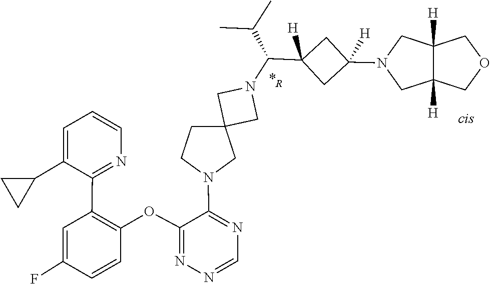 [SMILES: CC(C)[C@H]([C@H]1C[C@H](N2C[C@H]3COC[C@H]3C2)C1)N1CC2(CCN(c3ncnnc3Oc3ccc(F)cc3-c3ncccc3C3CC3)C2)C1] |
| Compound 151 | 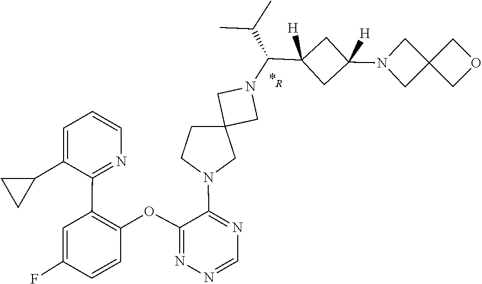 [SMILES: CC(C)[C@H]([C@H]1C[C@@H](N2CC3(COC3)C2)C1)N1CC2(CCN(c3ncnnc3Oc3ccc(F)cc3-c3ncccc3C3CC3)C2)C1] |
| Compound 152 | 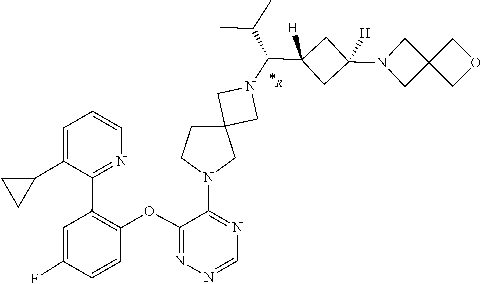 [SMILES: CC(C)[C@H]([C@H]1C[C@H](N2CC3(COC3)C2)C1)N1CC2(CCN(c3ncnnc3Oc3ccc(F)cc3-c3ncccc3C3CC3)C2)C1] |
| Compound 286 | 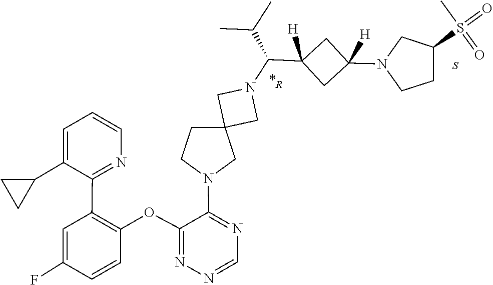 |
| Compound 288 | 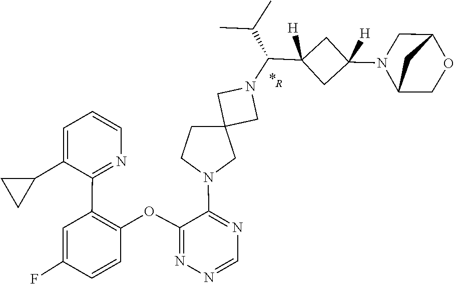 [SMILES: CC(C)[C@H]([C@H]1C[C@@H](N2C[C@H]3C[C@@H]2CO3)C1)N1CC2(CCN(c3ncnnc3Oc3ccc(F)cc3-c3ncccc3C3CC3)C2)C1] |

### Preparation of Compound 26

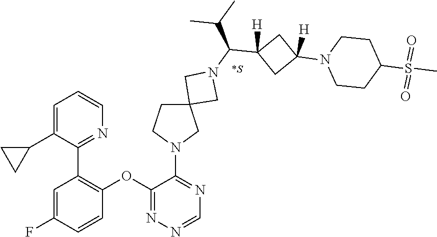 [SMILES: CC(C)[C@@H]([C@H]1C[C@@H](N2CCC(S(C)(=O)=O)CC2)C1)N1CC2(CCN(c3ncnnc3Oc3ccc(F)cc3-c3ncccc3C3CC3)C2)C1]

NaBH₃CN (23 mg; 0.37 mmol) was added to a mixture of intermediate 52b (*S) (100 mg; 0.2 mmol), 4-(methylsulfonyl)piperidine (165 mg; 1.01 mmol), AcOH (11 μL; 0.18 mmol) in MeOH (8 mL). Then, the reaction mixture was heated at 60° C. for 24 h. The reaction mixture was cooled to RT, diluted with DCM and poured onto a 10% aqueous solution of K₂CO₃. The organic layer was extracted with DCM (3×), dried over MgSO₄, filtered and evaporated till dryness. The residue was purified by chromatography over silica gel (mobile phase: gradient from 0% NH₄OH, 0% MeOH, 100% DCM to 0.1% NH₄OH, 5% MeOH, 95% DCM). The pure fractions were collected and evaporated to dryness. The residue (90 mg) was freeze-dried with water-ACN to give 77 mg (61%) of compound 26 as a white solid.

The compounds reported below were prepared following an analogous methodology as the one reported for the preparation of compound 26, starting from the appropriate starting materials (e.g. intermediate 52b (*S) or any other relevant intermediates):

| Compound Number | Structure |
|---|---|
| Compound 153 | 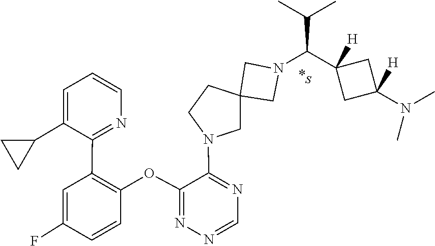 [SMILES: CC(C)[C@@H]([C@H]1C[C@@H](N(C)C)C1)N1CC2(CCN(c3ncnnc3Oc3ccc(F)cc3-c3ncccc3C3CC3)C2)C1] |
| Compound 285 | 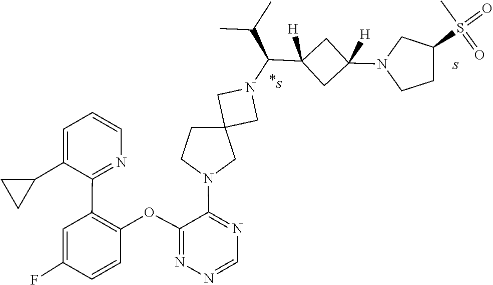 |
| Compound 287 | 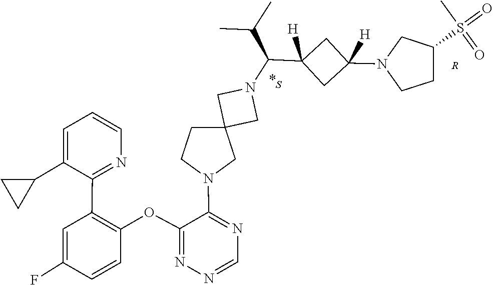 |

### Preparation of Compound 159 and Compound 160

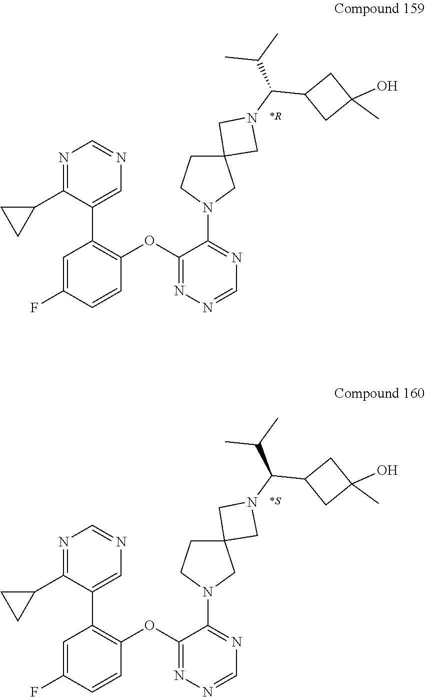 [SMILES: CC(C)[C@H](C1CC(C)(O)C1)N1CC2(CCN(c3ncnnc3Oc3ccc(F)cc3-c3cncnc3C3CC3)C2)C1 ; CC(C)[C@@H](C1CC(C)(O)C1)N1CC2(CCN(c3ncnnc3Oc3ccc(F)cc3-c3cncnc3C3CC3)C2)C1]

A solution of tetrabutylammonium fluoride (0.7 mL, 0.7 mmol, 1 M) was added dropwise to a solution of intermediate 5 (94 mg, 0.14 mmol) in THE (3 mL) at room temperature. The reaction mixture was stirred at room temperature overnight. The mixture was poured into ice water and EtOAc was added. The mixture was basified with a solution of K₂CO₃ 10% and the organic layer was separated, washed with brine, dried over MgSO₄, filtered and the solvent was evaporated to give 106 mg of residue. The residue was purified by chromatography over silica gel (Mobile phase: Gradient from 98% DCM, 2% MeOH (+10% NH₄OH) to 90% DCM, 10% MeOH (+10% NH₄OH)). The product containing fractions were collected and evaporated to dryness. The resulting compound (59 mg) was separated by chiral SFC (Stationary phase: CHIRALPAK AD-H 5 μm 250*21.2 mm, Mobile phase: 75% CO₂, 25% iPOH (0.3% iPrNH₂)). The products containing fractions were collected, evaporated to dryness to give 26 mg which were freeze-dried with water-ACN to give 22 mg (28%) of compound 159 and 24 mg which were freeze-dried with water-ACN to give 21 mg (27%) of compound 160 as a white solid.

### Preparation of Compound 161 and compound 162

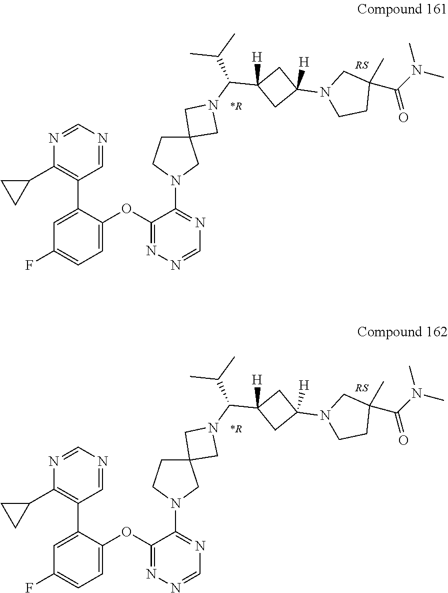 [SMILES: CC(C)[C@H]([C@H]1C[C@@H](N2CCC(C)(C(=O)N(C)C)C2)C1)N1CC2(CCN(c3ncnnc3Oc3ccc(F)cc3-c3cncnc3C3CC3)C2)C1 ; CC(C)[C@H]([C@H]1C[C@H](N2CCC(C)(C(=O)N(C)C)C2)C1)N1CC2(CCN(c3ncnnc3Oc3ccc(F)cc3-c3cncnc3C3CC3)C2)C1]

To a solution of intermediate 67 (250 mg, 0.38 mmol) in DMF (15 mL) was added T₃P (0.5 mL, 0.75 mmol, 50% purity) and Et₃N (0.16 mL, 1.13 mmol). The mixture was stirred at room temperature for 12 h. The mixture was diluted with EtOAc. The mixture was washed with saturated NaHCO₃, brine, dried over Mg₂SO₄, filtered and concentrated under reduced pressure. The residue was purified by chromatography over silica gel mobile phase: gradient from 0.1% NH₄OH, 0% MeOH, 100% DCM to 0.1% NH₄OH, 10% MeOH, 90% DCM). The pure fractions were collected and the solvent was evaporated under vacuum. The residue (300 mg) was purified by reverse phase (Stationary phase: YMC-actus Triart C18 10 μm 30*150 mm, Mobile phase: Gradient from 40% NH₄HCO₃ 0.2% pH=9.5, 35% ACN, 70% MeOH to 20% NH₄HCO₃ 0.2% pH=10, 40% ACN, 40% MeOH). The pure fractions were collected and the solvent was evaporated under vacuum.

Compound 161 was partitioned between acetonitrile (2 mL) and water (8 mL). The solution was lyophilized to dryness to give 58 mg of compound 161 as a white solid.

Compound 162 was partitioned between acetonitrile (2 mL) and water (8 mL). The solution was lyophilized to dryness to give 14 mg of compound 162 as a white solid.

### Preparation of Compound 166, Compound 167 and Compound 168

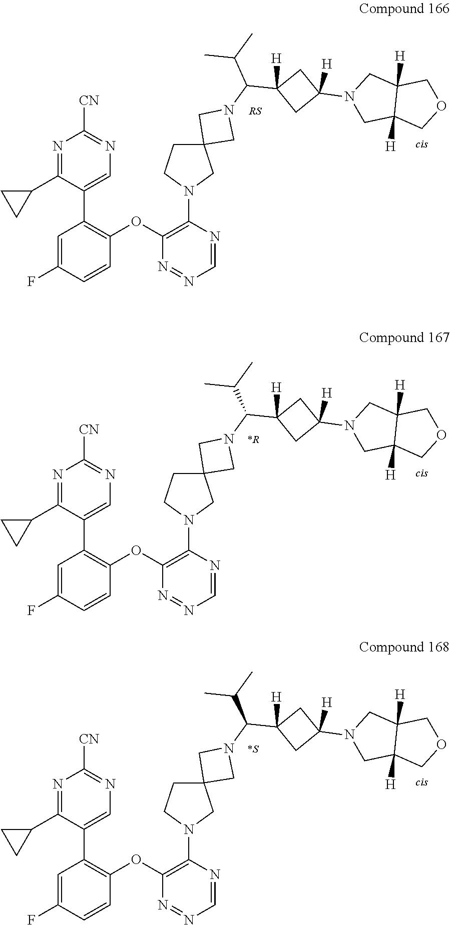 [SMILES: CC(C)C([C@H]1C[C@@H](N2C[C@H]3COC[C@H]3C2)C1)N1CC2(CCN(c3ncnnc3Oc3ccc(F)cc3-c3cnc(C#N)nc3C3CC3)C2)C1 ; CC(C)[C@H]([C@H]1C[C@@H](N2C[C@H]3COC[C@H]3C2)C1)N1CC2(CCN(c3ncnnc3Oc3ccc(F)cc3-c3cnc(C#N)nc3C3CC3)C2)C1 ; CC(C)[C@@H]([C@H]1C[C@@H](N2C[C@H]3COC[C@H]3C2)C1)N1CC2(CCN(c3ncnnc3Oc3ccc(F)cc3-c3cnc(C#N)nc3C3CC3)C2)C1]

NaBH₃CN (92 mg; 1.46 mmol) was added to a mixture of intermediate 75 (414 mg; 0.73 mmol), hexahydro-1H-furo[3,4-C]pyrrole (240 μL; 2.19 mmol) and AcOH (41 μL; 0.72 mmol) in MeOH (19 mL). The reaction mixture was stirred at 60° C. for 6 hours. The reaction mixture was poured onto a 10% aqueous solution of K₂CO₃ and EtOAc. The mixture was extracted with EtOAc (3×). The organic layer was dried over MgSO₄, filtered and the solvent was evaporated. The residue (610 mg) was purified by chromatography over silica gel (mobile phase: gradient from 0% NH₄OH, 0% MeOH, 100% DCM to 0.1% NH₄OH, 10% MeOH, 90% DCM). The pure fractions were collected and evaporated to dryness to give 140 mg of compound 166 and 82 mg of an impure fraction. Compound 166 (140 mg) was separated via chiral SFC (Stationary phase: CHIRALPAK AD-H 5 μm 250*30 mm, Mobile phase: 80% CO₂, 20% mixture of EtOH/iPrOH 50/50 v/v (+0.3% iPrNH₂)). The pure fractions were collected and evaporated to dryness yielding 12 mg of compound 167 which was freeze-dried with MeCN/water 20/80 to give 11 mg (2%) of compound 167 as a white powder and 12 mg of compound 168 which was freeze-dried with MeCN/water 20/80 to give 11 mg (2%) of compound 168 as a white powder. 82 mg of the impure fraction of compound 166 were purified by reverse phase (Stationary phase: YMC-actus Triart C18 10 μm 30*150 mm, Mobile phase: Gradient from 40% NH₄HCO₃ 0.2% pH=9.5, 30% ACN, 30% MeOH to 20% NH₄HCO₃ 0.2% pH=9.5, 40% ACN, 40% MeOH). The pure fractions were collected and evaporated to dryness giving additional 4 mg of the compound 166.

The compound reported below was prepared following an analogous methodology as described for compound 167 starting from intermediate 75:

| Compound Number | Structure |
|---|---|
| Compound 169 | 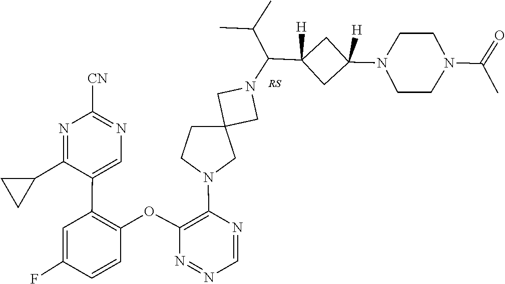 [SMILES: CC(=O)N1CCN([C@H]2C[C@@H](C(C(C)C)N3CC4(CCN(c5ncnnc5Oc5ccc(F)cc5-c5cnc(C#N)nc5C5CC5)C4)C3)C2)CC1] |

### Preparation of Compound 195

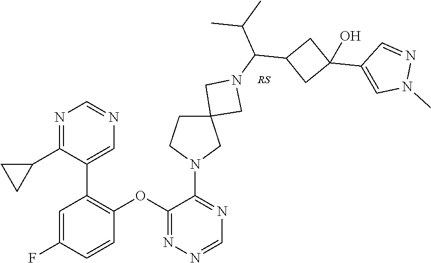 [SMILES: CC(C)C(C1CC(O)(c2cnn(C)c2)C1)N1CC2(CCN(c3ncnnc3Oc3ccc(F)cc3-c3cncnc3C3CC3)C2)C1]

To a solution of 4-iodo-1-methyl-1H-pyrazole (78 mg; 0.375 mmol) in tetrahydrofuran (2 mL) was added dropwise n-butyllithium (2.5 M in hexane) (0.16 mL; 0.375 mmol) at −70° C. After stirring for 20 minutes at −70° C., the reaction solution was added dropwise a solution of intermediate 38 (200 mg; 0.341 mmol, purity 92%) in tetrahydrofuran (2 mL) and stirred for 1 hour at −70° C. The reaction mixture was quenched with a saturated solution of NH₄Cl and extracted with ethyl acetate. The combined organic layers were washed with water then brine, dried over anhydrous sodium sulfate, filtered and evaporated to dryness. The residue was purified by reverse phase chromatography: (SunFire C18 OBD, 5 μm, 19×250 mm; mobile Phase A: Water (0.1% NH₄CO₃), mobile Phase B: ACN; Flow rate: 20 mL/min; gradient: 15% B to 40% B in 11 min) to afford 9.6 mg (4%) of compound 195 as a light yellow solid.

### Preparation of Compound 196

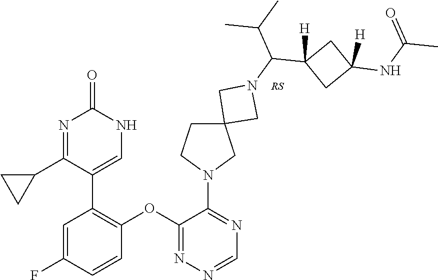 [SMILES: CC(=O)N[C@H]1C[C@@H](C(C(C)C)N2CC3(CCN(c4ncnnc4Oc4ccc(F)cc4-c4c[nH]c(=O)nc4C4CC4)C3)C2)C1]

To a solution of Compound 500 (350 mg; 0.568 mmol) in acetonitrile (17 mL) was added TMSI (1.6 g; 11.350 mmol). The reaction mixture was stirred for 2 h at room temperature, quenched with water (20 mL), adjusted to pH 8-9 with NaOH solution (1 N) and extracted with 8×10 mL of dichloromethane. The organic layers were combined, dried and concentrated under reduced pressure. The resulting crude product was purified by reverse phase chromatography (XBridge Prep OBD C18 Column, 19×250 mm, 5 um; mobile phase A: Water (10 mmol/L NH₄HCO₃+0.1% NH₄OH), mobile phase B: ACN; Flow rate: 20 mL/min; gradient: 30% B to 36% B in 7.5 min). The product fractions were concentrated and lyophilized to give 95.3 mg (26%) of compound 196 as an off white solid.

### Preparation of Compounds 197 (RS), 197a (*R) and 197b (*S

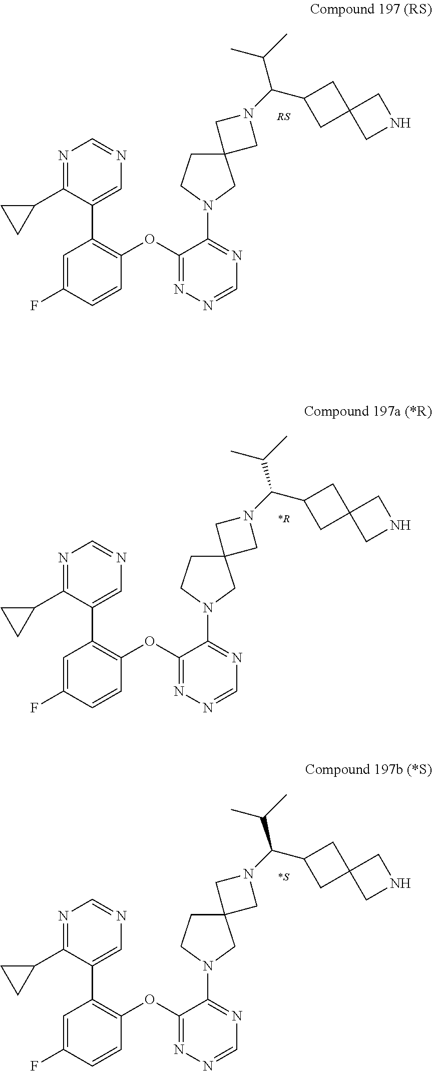

At 0° C., TFA (4.5 mL; 58.1 mmol) was added to a solution of intermediate 63 (1.95 g; 2.9 mmol) in DCM (40 mL). The reaction mixture was stirred overnight at room temperature. The solvents were evaporated and the residue was dissolved in water. Then, the solution was basified with a solution of NaOH 1 M until pH=9-10. After stirring for 10 minutes at room temperature, the resulting mixture was extracted with DCM (3×). The combined organics layers were washed with brine and dried over MgSO₄, filtered and evaporated till dryness. The residue (2 g) was purified by chromatography over silica gel (Mobile phase: Gradient from 95% DCM, 5% MeOH (+10% NH4OH) to 90% DCM, 10% MeOH (+10% NH4OH)). The pure fractions were collected, and the solvent was evaporated. The compound 197 (RS) (1.13 g, 68%) was purified by chiral SFC (Stationary phase: CHIRALPAK AD-H 5 μm 250*30 mm, Mobile phase: 55% CO₂, 45% mixture of ACN/iPrOH 20/80 v/v (+0.3% iPrNH₂)). The pure fractions were collected and the solvent was evaporated till dryness to give 405 mg (25%) of compound 197 (*R) and 388 mg (24%) of compound 197b (*S).

### Preparation of Compound 198a

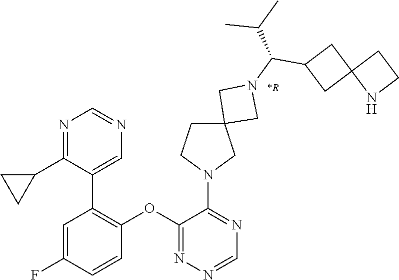 [SMILES: CC(C)[C@H](C1CC2(CCN2)C1)N1CC2(CCN(c3ncnnc3Oc3ccc(F)cc3-c3cncnc3C3CC3)C2)C1]

At 0° C., TFA (0.4 mL; 4.2 mmol) was added to a solution of Compound 496a (*R) (143 mg; 0.2 mmol) in DCM (40 mL). The reaction mixture was stirred overnight at room temperature. The solvents were evaporated and the residue was dissolved in water. Then, the solution was basified with a solution of NaOH 1 M until pH=9-10. After stirring for 10 minutes at room temperature, The resulting mixture was extracted with DCM (3×). The combined organics layers were washed with brine and dried over MgSO₄, filtered and evaporated till dryness. The residue (2 g) was purified by chromatography over silica gel (Mobile phase: Gradient from 95% DCM, 5% MeOH (+10% NH₄OH) to 90% DCM, 10% MeOH (+10% NH₄OH)). The pure fractions were collected, and the solvent was evaporated to give 136 mg of compound 198a.

The compounds reported below were prepared following an analogous methodology starting from Compound 496b (*S)

| Compound Number | Structure |
|---|---|
| Compound 198b | 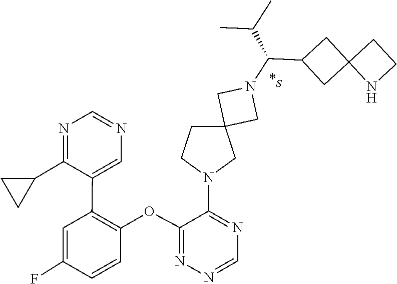 [SMILES: CC(C)[C@H](C1CC2(CCN2)C1)N1CC2(CCN(c3ncnnc3Oc3ccc(F)cc3-c3cncnc3C3CC3)C2)C1] |
| Compound 198 | 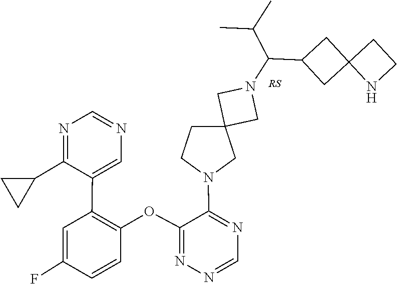 [SMILES: CC(C)C(C1CC2(CCN2)C1)N1CC2(CCN(c3ncnnc3Oc3ccc(F)cc3-c3cncnc3C3CC3)C2)C1] |

### Preparation of Compound 199

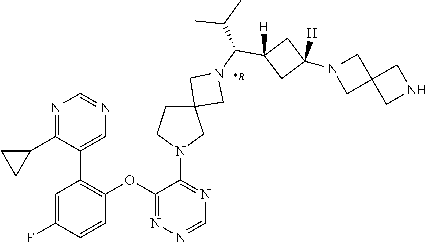 [SMILES: CC(C)[C@H]([C@H]1C[C@@H](N2CC3(CNC3)C2)C1)N1CC2(CCN(c3ncnnc3Oc3ccc(F)cc3-c3cncnc3C3CC3)C2)C1]

TFA (1.96 mL; 25.62 mmol) was added to a solution of Compound 497 (930 mg; 1.28 mmol) in DCM (25 mL) at 0° C. and the reaction mixture was stirred at room temperature for 18 h. The residue was dissolved in DCM and basified with a 30% aqueous solution of NH₄OH at 0-5° C. The mixture was stirred at rt for 1 h. The mixture was filtered through Chromabond® and the filtrate was evaporated to give 802 mg of compound 199 (quantitative) as a white foam.

### Preparation of Compound 199a

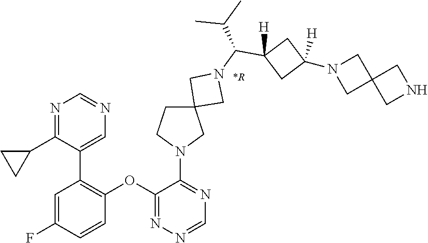 [SMILES: CC(C)[C@H]([C@H]1C[C@H](N2CC3(CNC3)C2)C1)N1CC2(CCN(c3ncnnc3Oc3ccc(F)cc3-c3cncnc3C3CC3)C2)C1]

TFA (106 μL; 1.38 mmol) was added to a solution of Compound 498 (50 mg; 0.069 mmol) in DCM (1.5 mL) at 0° C. and the reaction mixture was stirred at room temperature for 18 h. The residue was dissolved in DCM and basified with a 30% aqueous solution of NH₄OH at 0-5° C. The mixture was stirred at rt for 1 h. The mixture was filtered through Chromabond® and the filtrate was evaporated to give 47 mg of compound 199a (quantitative) as a white foam.

The compounds reported below were prepared following an analogous methodology as described for Compound 199 starting from the corresponding intermediates:

| Compound Number | Structure |
|---|---|
| Compound 334 | 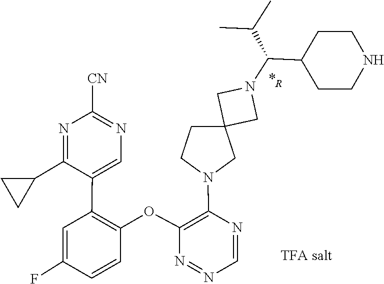 [SMILES: CC(C)[C@H](C1CCNCC1)N1CC2(CCN(c3ncnnc3Oc3ccc(F)cc3-c3cnc(C#N)nc3C3CC3)C2)C1] |
| | from intermediate 73 |
| Compound 337 | 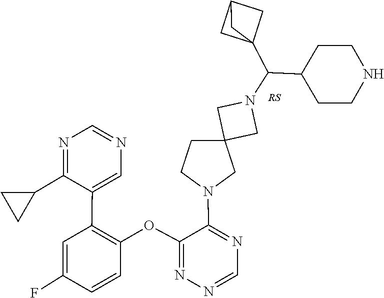 [SMILES: Fc1ccc(Oc2nncnc2N2CCC3(C2)CN(C(C2CCNCC2)C24CC(C2)C4)C3)c(-c2cncnc2C2CC2)c1] |
| | from intermediate 251 |
| Compound 352 | 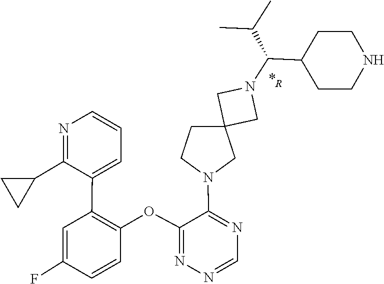 [SMILES: CC(C)[C@H](C1CCNCC1)N1CC2(CCN(c3ncnnc3Oc3ccc(F)cc3-c3cccnc3C3CC3)C2)C1] |
| | from intermediate 44 |
| Compound 367 | 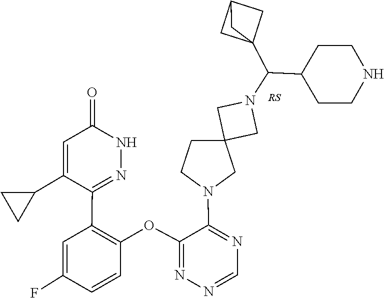 [SMILES: O=c1cc(C2CC2)c(-c2cc(F)ccc2Oc2nncnc2N2CCC3(C2)CN(C(C2CCNCC2)C24CC(C2)C4)C3)n[nH]1] |
| | from intermediate 302 |
| Compound 368 | 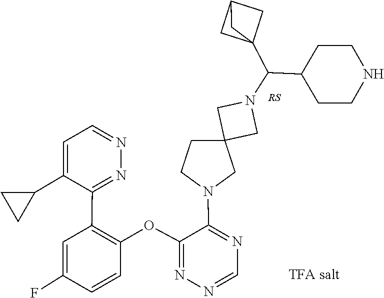 [SMILES: Fc1ccc(Oc2nncnc2N2CCC3(C2)CN(C(C2CCNCC2)C24CC(C2)C4)C3)c(-c2nnccc2C2CC2)c1] |
| | from intermediate 297 |
| Compound 355 | 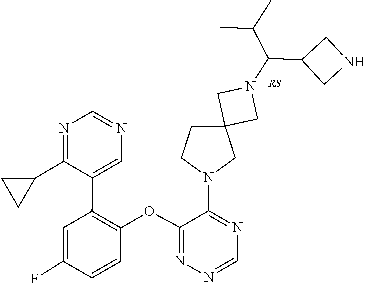 [SMILES: CC(C)C(C1CNC1)N1CC2(CCN(c3ncnnc3Oc3ccc(F)cc3-c3cncnc3C3CC3)C2)C1] |
| | from intermediate 33 |

The compounds in the table below were prepared using an analogous method as described for the preparation of intermediate 29 starting from the corresponding starting materials.

| Compound number | Structure |
|---|---|
| Compound 202 | 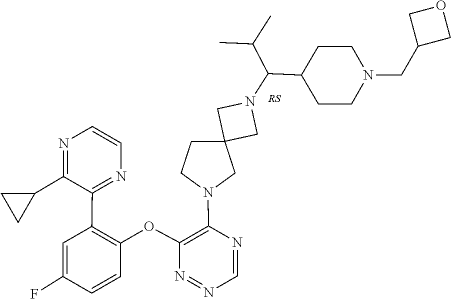 [SMILES: CC(C)C(C1CCN(CC2COC2)CC1)N1CC2(CCN(c3ncnnc3Oc3ccc(F)cc3-c3nccnc3C3CC3)C2)C1] |
| | Using intermediate 88 |
| Compound 203 | 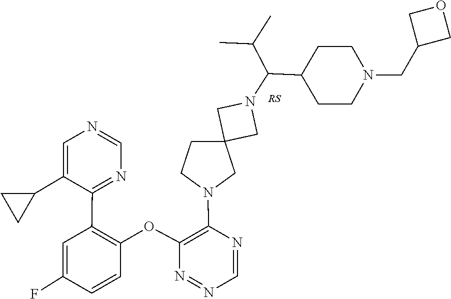 [SMILES: CC(C)C(C1CCN(CC2COC2)CC1)N1CC2(CCN(c3ncnnc3Oc3ccc(F)cc3-c3ncncc3C3CC3)C2)C1] |
| | Using intermediate 93 |
| Compound 335 | 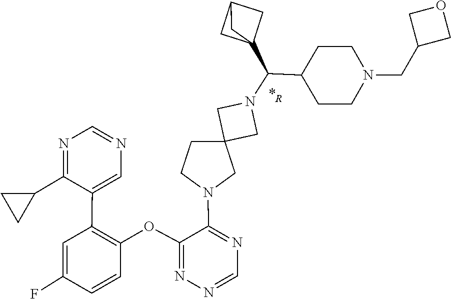 [SMILES: Fc1ccc(Oc2nncnc2N2CCC3(C2)CN([C@H](C2CCN(CC4COC4)CC2)C24CC(C2)C4)C3)c(-c2cncnc2C2CC2)c1] |
| Compound 336 | 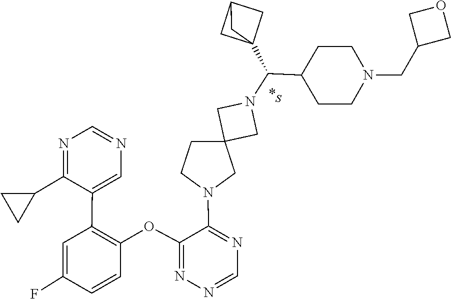 [SMILES: Fc1ccc(Oc2nncnc2N2CCC3(C2)CN([C@@H](C2CCN(CC4COC4)CC2)C24CC(C2)C4)C3)c(-c2cncnc2C2CC2)c1] |
| | from Compound 337, SFC separation: DAICEL CHIRALPAK |
| | AS-H (250 mm x 30 mm, 5 um); Mobile phase: A: Supercritical |
| | CO₂, B: Neu-EtOH, A:B = 55:45 at 80 mL/min |
| Compound 339 | 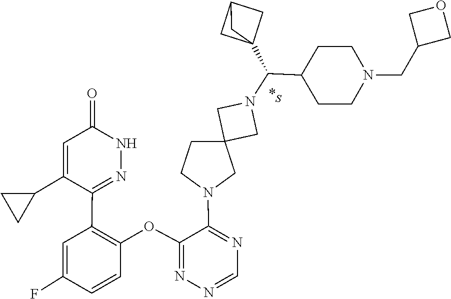 [SMILES: O=c1cc(C2CC2)c(-c2cc(F)ccc2Oc2nncnc2N2CCC3(C2)CN([C@@H](C2CCN(CC4COC4)CC2)C24CC(C2)C4)C3)n[nH]1] |
| | from Compound 367, SFC separation: DAICEL CHIRALPAK |
| | IG (250 mm * 30 mm, 10 um); Mobile phase: A: Supercritical CO₂, |
| | B: 0.1% NH₃H₂O EtOH, A:B = 45:55 at 80 mL/min |
| Compound 340 | 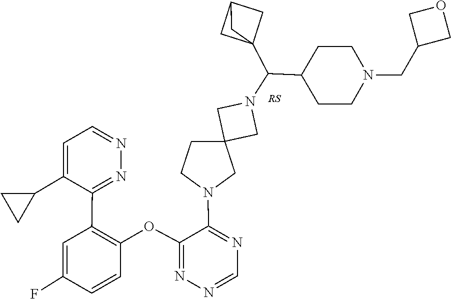 [SMILES: Fc1ccc(Oc2nncnc2N2CCC3(C2)CN(C(C2CCN(CC4COC4)CC2)C24CC(C2)C4)C3)c(-c2nnccc2C2CC2)c1] |
| | from Compound 368 |
| Compound 333 | 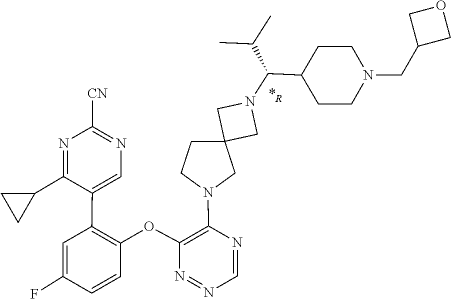 [SMILES: CC(C)[C@H](C1CCN(CC2COC2)CC1)N1CC2(CCN(c3ncnnc3Oc3ccc(F)cc3-c3cnc(C#N)nc3C3CC3)C2)C1] |
| | from Compound 334 |

### Preparation of Compound 154

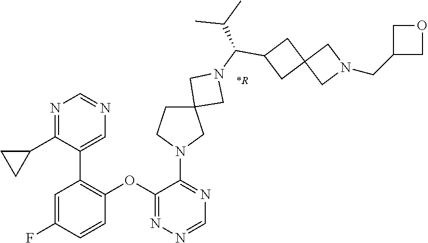 [SMILES: CC(C)[C@H](C1CC2(C1)CN(CC1COC1)C2)N1CC2(CCN(c3ncnnc3Oc3ccc(F)cc3-c3cncnc3C3CC3)C2)C1]

NaBH₃CN (85 mg; 0.4 mmol) was added to a mixture of compound 197a (*R) (152 mg; 0.27 mmol), oxetane-3-carbaldehyde (24 μL; 0.35 mmol; 2 M) in MeOH (6 mL). The reaction mixture was stirred at room temperature for 2 hours. The reaction mixture was diluted with EtOAc and poured onto a 10% aqueous solution of K₂CO₃. The organic layer was extracted with EtOAc, dried over MgSO₄, filtered and evaporated till dryness. The residue (177 mg) was purified by chromatography over silica gel (mobile phase: gradient from 0.1% NH₄OH, 5% MeOH, 95% DCM to 0.1% NH₄OH, 10% MeOH, 90% DCM). The pure fractions were collected, evaporated to dryness and freeze-dried with a mixture of water-ACN to give 75 mg (45%) of compound 154 as a white solid.

The compounds reported below were prepared following an analogous methodology as reported for the preparation of compound 154 starting from the corresponding compounds:

| Compound number | Structure |
|---|---|
| Compound 155 | 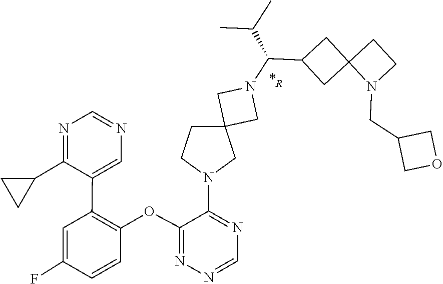 [SMILES: CC(C)[C@H](C1CC2(CCN2CC2COC2)C1)N1CC2(CCN(c3ncnnc3Oc3ccc(F)cc3-c3cncnc3C3CC3)C2)C1] |
| | From compound 198a |
| Compound 156 | 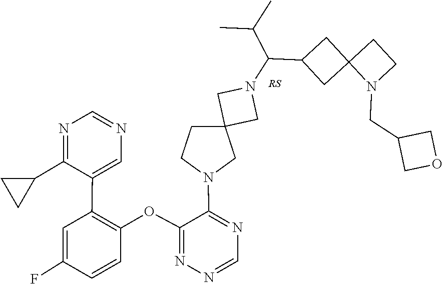 [SMILES: CC(C)C(C1CC2(CCN2CC2COC2)C1)N1CC2(CCN(c3ncnnc3Oc3ccc(F)cc3-c3cncnc3C3CC3)C2)C1] |
| | From compound 198 |

### Preparation of Compound 157

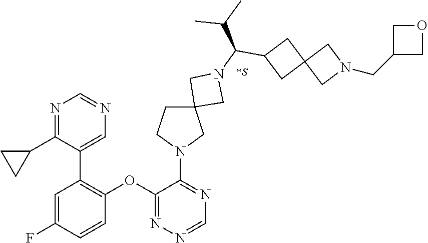 [SMILES: CC(C)[C@@H](C1CC2(C1)CN(CC1COC1)C2)N1CC2(CCN(c3ncnnc3Oc3ccc(F)cc3-c3cncnc3C3CC3)C2)C1]

NaBH₃CN (85 mg; 0.4 mmol) was added to a mixture of compound 197b (*S) (152 mg; 0.27 mmol), oxetane-3-carbaldehyde (24 μL; 0.35 mmol; 2 M) in MeOH (6 mL). The reaction mixture was stirred at room temperature for 2 hours. The reaction mixture was diluted with EtOAc and poured onto a 10% aqueous solution of K₂CO₃. The organic layer was extracted with EtOAc, dried over MgSO₄, filtered and evaporated till dryness. The residue (175 mg) was purified by chromatography over silica gel (mobile phase: gradient from 0.1% NH₄OH, 5% MeOH, 95% DCM to 0.1% NH₄OH, 10% MeOH, 90% DCM). The pure fractions were collected and evaporated to dryness. was freeze-dried with a mixture of water-ACN to give 130 mg (76%) of final compound 157 as a white solid.

The compound reported below was prepared following an analogous methodology as described for compound 157, starting from the corresponding compounds:

| Compound number | Structure |
|---|---|
| Compound 158 | 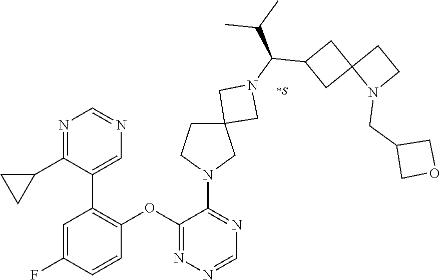 [SMILES: CC(C)[C@@H](C1CC2(CCN2CC2COC2)C1)N1CC2(CCN(c3ncnnc3Oc3ccc(F)cc3-c3cncnc3C3CC3)C2)C1] |
| | from compound 198b |
| Compound 353 | 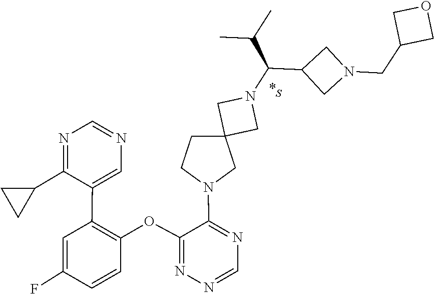 [SMILES: CC(C)[C@@H](C1CN(CC2COC2)C1)N1CC2(CCN(c3ncnnc3Oc3ccc(F)cc3-c3cncnc3C3CC3)C2)C1] |
| Compound 354 | 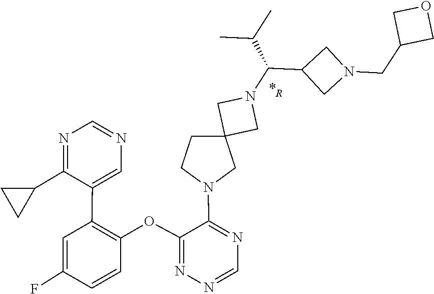 [SMILES: CC(C)[C@H](C1CN(CC2COC2)C1)N1CC2(CCN(c3ncnnc3Oc3ccc(F)cc3-c3cncnc3C3CC3)C2)C1] |
| | from Compound 355, SFC separation: DAICEL CHIRALPAK |
| | AD-H (250 mm x 30 mm, 5 um); mobile phase: A: Supercritical |
| | CO₂, B: 0.1% NH₃H₂O EtOH, eluent: A:B = 70:30 at 60 mL/min |

### Preparation of Compound 163

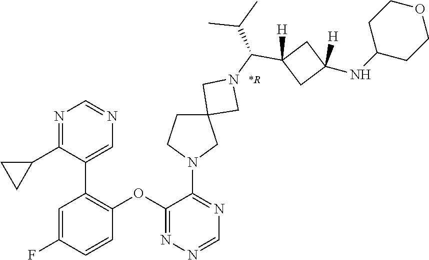 [SMILES: CC(C)[C@H]([C@H]1C[C@@H](NC2CCOCC2)C1)N1CC2(CCN(c3ncnnc3Oc3ccc(F)cc3-c3cncnc3C3CC3)C2)C1]

NaBH(OAc)₃ (175 mg; 0.83 mmol) was added to a mixture of compound 1a (300 mg; 0.55 mmol), tetrahydro-4H-pyran-4-one (60 μL; 0.67 mmol) in DCE (10 mL). Then, the reaction mixture was stirred at room temperature for 24 h. The reaction mixture was diluted with DCM and poured onto a 10% aqueous solution of K₂CO₃. The organic layer was extracted with DCM (3×), dried over MgSO₄, filtered and evaporated till dryness. The residue (350 mg) was purified by chromatography over silica gel (mobile phase: gradient from 0% NH₄OH, 0% MeOH, 100% DCM to 0.7% NH₄OH, 10% MeOH, 90% DCM). The pure fractions were collected and evaporated to dryness to give 230 mg (66%) of compound 163.

### Preparation of Compound 164

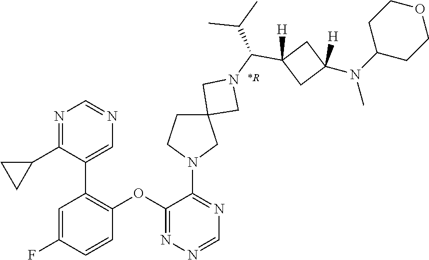 [SMILES: CC(C)[C@H]([C@H]1C[C@@H](N(C)C2CCOCC2)C1)N1CC2(CCN(c3ncnnc3Oc3ccc(F)cc3-c3cncnc3C3CC3)C2)C1]

NaBH(OAc)₃ (95 mg; 0.45 mmol) was added to a mixture of compound 163 (57 mg; 0.09 mmol), formaldehyde 37% in water (95 μL; 0.94 mmol) and molecular sieves 4 A (60 mg) in DCE (10 mL). Then, the reaction mixture was stirred at room temperature for 2 h. The reaction mixture was diluted with DCM and poured onto a 10% aqueous solution of K₂CO₃. The solution was filtered through a pad of Celite® The organic layer was extracted with DCM, dried over MgSO₄, filtered and evaporated till dryness. The residue (44 mg) was purified by reverse phase (Stationary phase: YMC-actus Triart C18 10 μm 30*150 mm, Mobile phase: Gradient from 45% NH₄HCO₃ 0.2%, 55% ACN to 25% NH₄HCO₃ 0.2%, 75% ACN). The pure fractions were collected and evaporated to dryness. Compound 164 was partitioned between acetonitrile (2 mL) and water (8 mL). The solution was lyophilized to dryness to give 18 mg (31%) of compound 164 as a white solid.

The compounds reported below were prepared following an analogous methodology as described for Compound 164, starting from the corresponding compounds:

| Compound Number | Structure |
|---|---|
| Compound 348 | 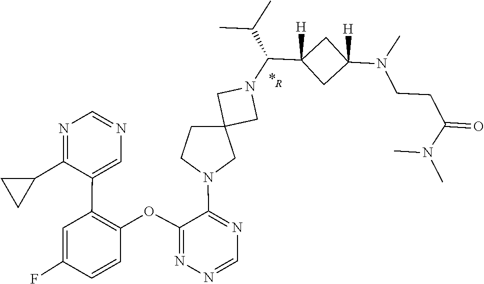 [SMILES: CC(C)[C@H]([C@H]1C[C@@H](N(C)CCC(=O)N(C)C)C1)N1CC2(CCN(c3ncnnc3Oc3ccc(F)cc3-c3cncnc3C3CC3)C2)C1] |
| | from Compound 347 |
| Compound 350 | 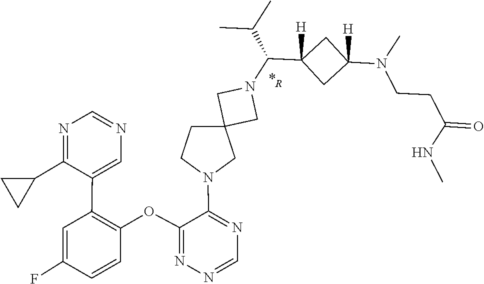 [SMILES: CNC(=O)CCN(C)[C@H]1C[C@@H]([C@@H](C(C)C)N2CC3(CCN(c4ncnnc4Oc4ccc(F)cc4-c4cncnc4C4CC4)C3)C2)C1] |
| | from Compound 349 |

### Preparation of Compound 165

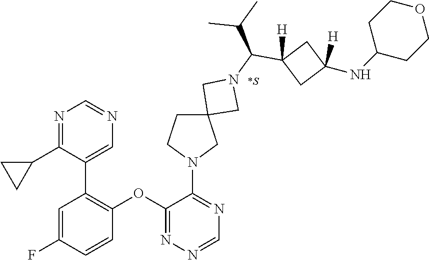 [SMILES: CC(C)[C@@H]([C@H]1C[C@@H](NC2CCOCC2)C1)N1CC2(CCN(c3ncnnc3Oc3ccc(F)cc3-c3cncnc3C3CC3)C2)C1]

NaBH(OAc)₃ (187 mg; 0.89 mmol) was added to a mixture of compound 1b (320 mg; 0.59 mmol), tetrahydro-4H-pyran-4-one (65 μL; 0.71 mmol) in DCE (12 mL). Then, the reaction mixture was stirred at room temperature for 24 h. The reaction mixture was diluted with DCM and poured onto a 10% aqueous solution of K₂CO₃. The organic layer was extracted with DCM (3×), dried over MgSO₄, filtered and evaporated till dryness. The residue (380 mg) was purified by chromatography over silica gel (mobile phase: gradient from 0% NH₄OH, 0% MeOH, 100% DCM to 0.7% NH₄OH, 10% MeOH, 90% DCM). The pure fractions were collected and evaporated to dryness to give 170 mg (47%) of compound 165.

### Preparation of Compound 170

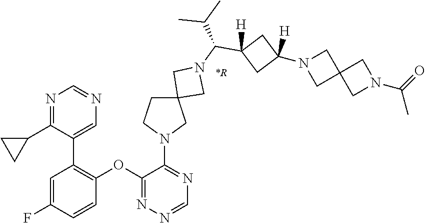 [SMILES: CC(=O)N1CC2(C1)CN([C@H]1C[C@@H]([C@@H](C(C)C)N3CC4(CCN(c5ncnnc5Oc5ccc(F)cc5-c5cncnc5C5CC5)C4)C3)C1)C2]

T₃P (1.14 mL; 1.92 mmol) was added to a solution of compound 199 (800 mg; 1.28 mmol), acetic acid (81 μL; 1.41 mmol) and DIPEA (1.10 mL; 6.39 mmol) in DCM (8.4 mL). The reaction mixture was stirred at rt for 18 h, poured into a 10% aqueous solution of NaHCO₃ and DCM. The mixture was filtered through Chromabond® and the filtrate was evaporated to dryness. The residue was purified by chromatography over silica gel (irregular SiOH, 40 g; mobile phase: gradient from 98% DCM, 2% MeOH to 92% DCM, 8% MeOH). The pure fractions were collected, evaporated to dryness and freeze dried (ACN/water) yielding 145 mg of compound 170 (17%) as a white solid.

¹H NMR (500 MHz, DMSO-d₆) δ ppm 8.93 (br s, 1H), 8.41 (br s, 2H), 7.57 (br s, 1H), 7.38-7.50 (m, 2H), 4.12 (s, 2H), 3.84 (s, 2H), 3.40-3.75 (m, 4H), 2.85-3.21 (m, 8H), 2.74-2.84 (m, 1H), 1.86-2.02 (m, 5H), 1.73-1.84 (m, 2H), 1.71 (s, 3H), 1.55-1.66 (m, 2H), 1.44 (q, J=9.7 Hz, 1H), 0.65-1.10 (m, 10H)

The compounds in the table below were prepared using an analogous method as described for the preparation of compound 170 starting from the corresponding compounds or intermediates.

| Compound number | Structure |
|---|---|
| Compound 171 | 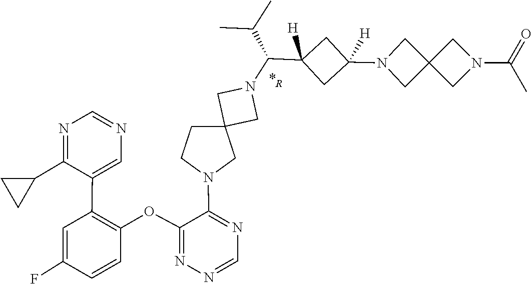 [SMILES: CC(=O)N1CC2(C1)CN([C@H]1C[C@H]([C@@H](C(C)C)N3CC4(CCN(c5ncnnc5Oc5ccc(F)cc5-c5cncnc5C5CC5)C4)C3)C1)C2] |
| | From compound 199a |
| Compound 172 | 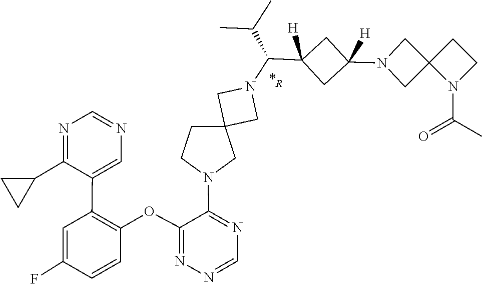 [SMILES: CC(=O)N1CCC12CN([C@H]1C[C@@H]([C@@H](C(C)C)N3CC4(CCN(c5ncnnc5Oc5ccc(F)cc5-c5cncnc5C5CC5)C4)C3)C1)C2] |
| | From intermediate 38 doing reductive amination with tert- |
| | butyl 1,6-diazaspiro[3.3]heptane-1-carboxylate hemioxalate |
| Compound 173 | 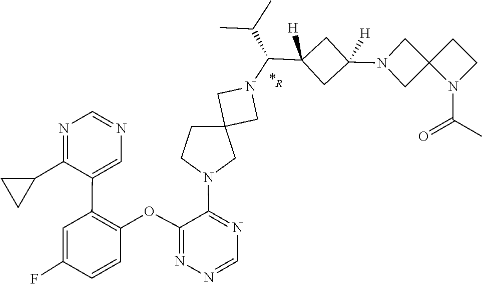 [SMILES: CC(=O)N1CCC12CN([C@H]1C[C@H]([C@@H](C(C)C)N3CC4(CCN(c5ncnnc5Oc5ccc(F)cc5-c5cncnc5C5CC5)C4)C3)C1)C2] |
| | From intermediate 38 doing reductive amination with tert- |
| | butyl 1,6-diazaspiro[3.3]heptane-1-carboxylate hemioxalate |
| Compound 174 | 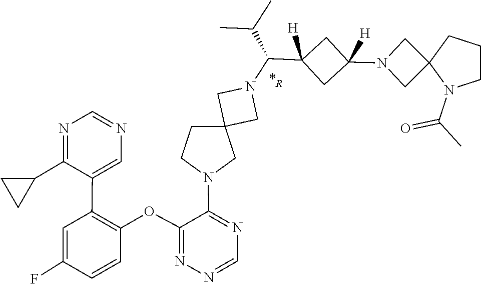 [SMILES: CC(=O)N1CCCC12CN([C@H]1C[C@@H]([C@@H](C(C)C)N3CC4(CCN(c5ncnnc5Oc5ccc(F)cc5-c5cncnc5C5CC5)C4)C3)C1)C2] |
| Compound 175 | 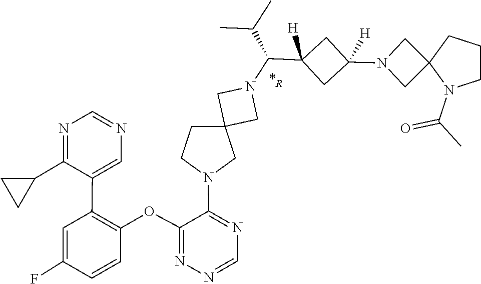 [SMILES: CC(=O)N1CCCC12CN([C@H]1C[C@H]([C@@H](C(C)C)N3CC4(CCN(c5ncnnc5Oc5ccc(F)cc5-c5cncnc5C5CC5)C4)C3)C1)C2] |
| | From intermediate 38 doing reductive amination with 2,5- |
| | Diazaspiro[3.4]octane-5-carboxylic acid tert-butyl ester |
| Compound 176 | 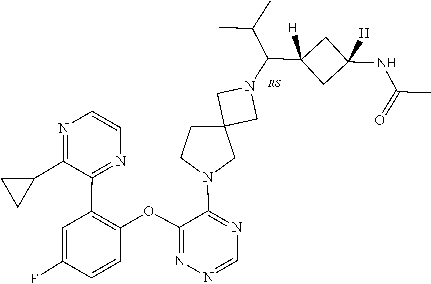 [SMILES: CC(=O)N[C@H]1C[C@@H](C(C(C)C)N2CC3(CCN(c4ncnnc4Oc4ccc(F)cc4-c4nccnc4C4CC4)C3)C2)C1] |
| Compound 177 | 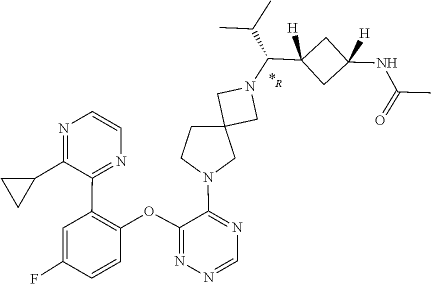 [SMILES: CC(=O)N[C@H]1C[C@@H]([C@@H](C(C)C)N2CC3(CCN(c4ncnnc4Oc4ccc(F)cc4-c4nccnc4C4CC4)C3)C2)C1] |
| Compound 178 | 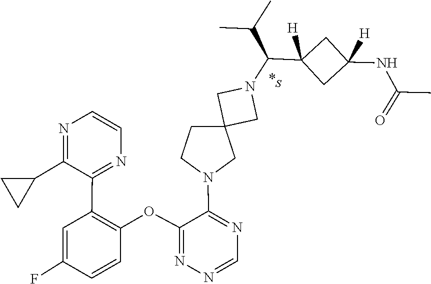 [SMILES: CC(=O)N[C@H]1C[C@@H]([C@H](C(C)C)N2CC3(CCN(c4ncnnc4Oc4ccc(F)cc4-c4nccnc4C4CC4)C3)C2)C1] |
| | from intermediate 88 using acetic anhydride as acylating |
| | agent. |
| | Chiral separation: (R,R)-WHELK-O1-Kromasil(02), |
| | 5 cm * 25 cm(5 um); mobile Phase A: 10 mM NH₃ in methyl tert- |
| | butyl ether (MTBE), Mobile Phase B: EtOH; Flow rate:20 |
| | mL/min; gradient:20% B for 22 min |
| Compound 179 | 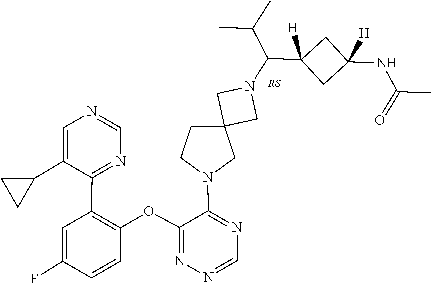 [SMILES: CC(=O)N[C@H]1C[C@@H](C(C(C)C)N2CC3(CCN(c4ncnnc4Oc4ccc(F)cc4-c4ncncc4C4CC4)C3)C2)C1] |
| Compound 180 | 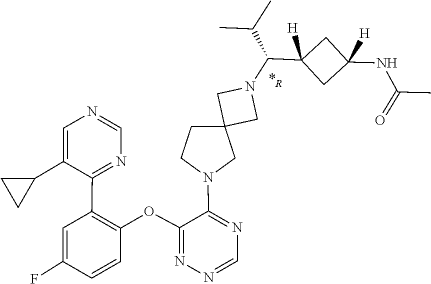 [SMILES: CC(=O)N[C@H]1C[C@@H]([C@@H](C(C)C)N2CC3(CCN(c4ncnnc4Oc4ccc(F)cc4-c4ncncc4C4CC4)C3)C2)C1] |
| Compound 181 | 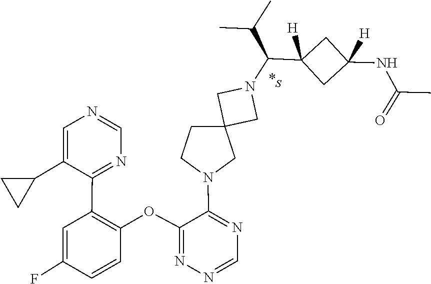 [SMILES: CC(=O)N[C@H]1C[C@@H]([C@H](C(C)C)N2CC3(CCN(c4ncnnc4Oc4ccc(F)cc4-c4ncncc4C4CC4)C3)C2)C1] |
| | from intermediate 93 Chiral separation : (R,R)Whelk-01, |
| | 21.1 * 250 mm, 5 um; mobile phase A:MTBE (10 mM NH₃— |
| | MeOH), mobile phase B: EtOH; flow rate:20 mL/min; 15% B |
| | for 34 min) |
| Compound 182 | 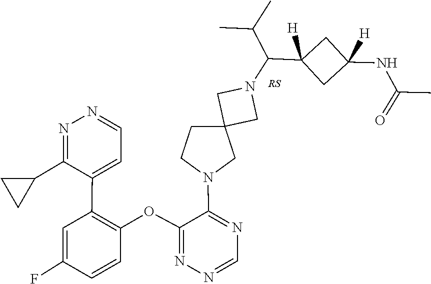 [SMILES: CC(=O)N[C@H]1C[C@@H](C(C(C)C)N2CC3(CCN(c4ncnnc4Oc4ccc(F)cc4-c4ccnnc4C4CC4)C3)C2)C1] |
| | from intermediate 101 |
| Compound 183 | 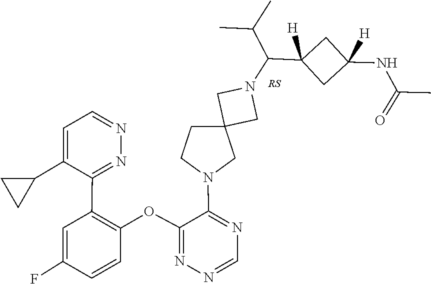 [SMILES: CC(=O)N[C@H]1C[C@@H](C(C(C)C)N2CC3(CCN(c4ncnnc4Oc4ccc(F)cc4-c4nnccc4C4CC4)C3)C2)C1] |
| | from intermediate 106 |
| Compound 304 | 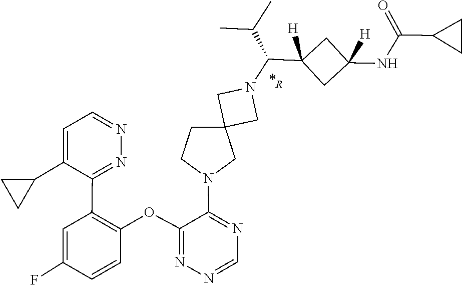 [SMILES: CC(C)[C@H]([C@H]1C[C@@H](NC(=O)C2CC2)C1)N1CC2(CCN(c3ncnnc3Oc3ccc(F)cc3-c3nnccc3C3CC3)C2)C1] |
| | from intermediate 106 |
| Compound 305 | 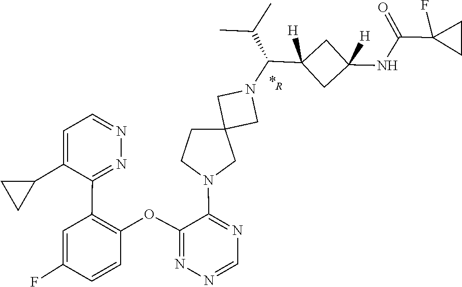 [SMILES: CC(C)[C@H]([C@H]1C[C@@H](NC(=O)C2(F)CC2)C1)N1CC2(CCN(c3ncnnc3Oc3ccc(F)cc3-c3nnccc3C3CC3)C2)C1] |
| | from intermediate 106 |
| Compound 184 | 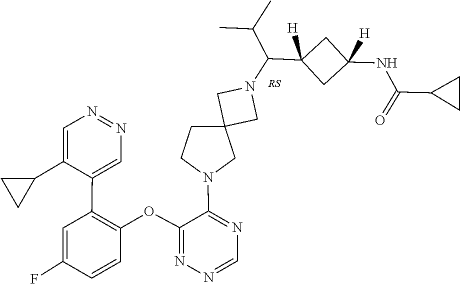 [SMILES: CC(C)C([C@H]1C[C@@H](NC(=O)C2CC2)C1)N1CC2(CCN(c3ncnnc3Oc3ccc(F)cc3-c3cnncc3C3CC3)C2)C1] |
| Compound 185 | 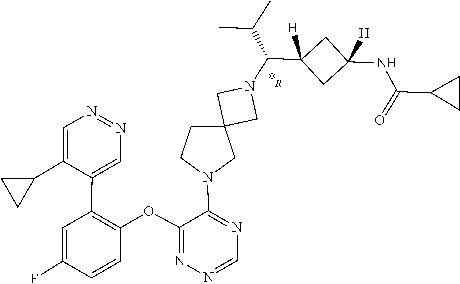 [SMILES: CC(C)[C@H]([C@H]1C[C@@H](NC(=O)C2CC2)C1)N1CC2(CCN(c3ncnnc3Oc3ccc(F)cc3-c3cnncc3C3CC3)C2)C1] |
| Compound 186 | 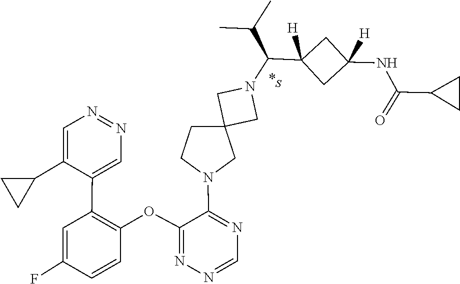 [SMILES: CC(C)[C@@H]([C@H]1C[C@@H](NC(=O)C2CC2)C1)N1CC2(CCN(c3ncnnc3Oc3ccc(F)cc3-c3cnncc3C3CC3)C2)C1] |
| | from intermediate 109 Chiral Prep-HPLC (CHIRAL ART |
| | Cellulose-SB, 2 x 25 mm, 5 um; mobile Phase A:Hex (8 |
| | mmol/L NH₃.MeOH), mobile Phase B:EtOH; Flow rate:20 |
| | mL/min; 35% B for 13 min) |
| Compound 187 | 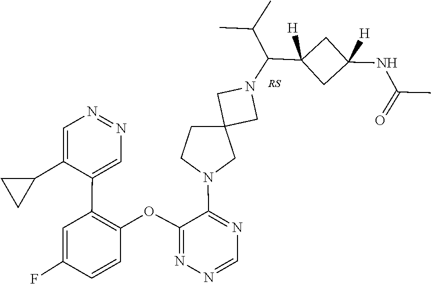 [SMILES: CC(=O)N[C@H]1C[C@@H](C(C(C)C)N2CC3(CCN(c4ncnnc4Oc4ccc(F)cc4-c4cnncc4C4CC4)C3)C2)C1] |
| | from intermediate 109 |
| Compound 188 | 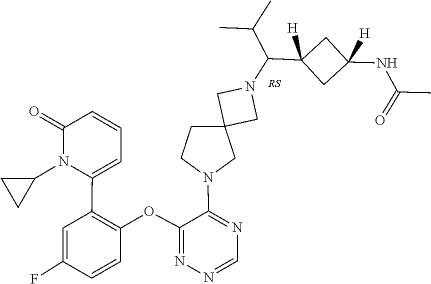 [SMILES: CC(=O)N[C@H]1C[C@@H](C(C(C)C)N2CC3(CCN(c4ncnnc4Oc4ccc(F)cc4-c4cccc(=O)n4C4CC4)C3)C2)C1] |
| | from intermediate 113 |
| Compound 189 | 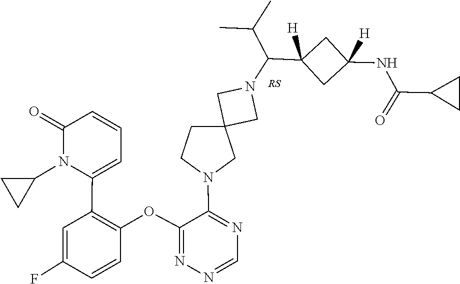 [SMILES: CC(C)C([C@H]1C[C@@H](NC(=O)C2CC2)C1)N1CC2(CCN(c3ncnnc3Oc3ccc(F)cc3-c3cccc(=O)n3C3CC3)C2)C1] |
| Compound 190 | 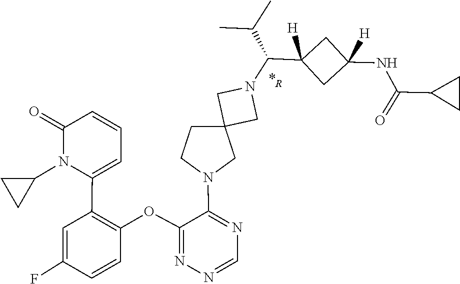 [SMILES: CC(C)[C@H]([C@H]1C[C@@H](NC(=O)C2CC2)C1)N1CC2(CCN(c3ncnnc3Oc3ccc(F)cc3-c3cccc(=O)n3C3CC3)C2)C1] |
| Compound 191 | 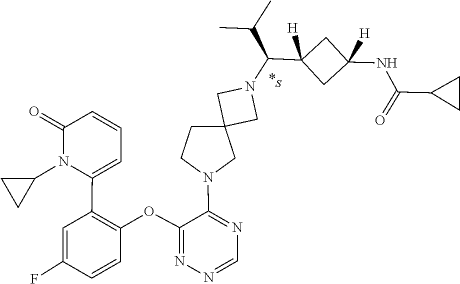 [SMILES: CC(C)[C@@H]([C@H]1C[C@@H](NC(=O)C2CC2)C1)N1CC2(CCN(c3ncnnc3Oc3ccc(F)cc3-c3cccc(=O)n3C3CC3)C2)C1] |
| | from intermediate 113 Chiral separation ((R,R)-Whelk-01, |
| | 2.12 * 25 cm, 5 um; mobile mhase A: 2 mM NH₃ in methyl tert- |
| | butyl ether (MTBE), mobile phase B: EtOH; Flow rate:20 |
| | mL/min; 20% B for 26 min) |

### Preparation of Compound 192

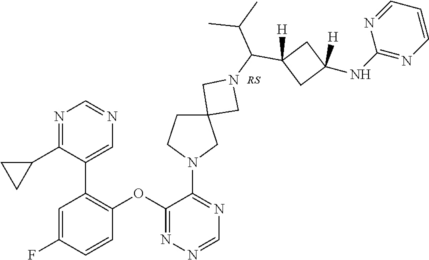 [SMILES: CC(C)C([C@H]1C[C@@H](Nc2ncccn2)C1)N1CC2(CCN(c3ncnnc3Oc3ccc(F)cc3-c3cncnc3C3CC3)C2)C1]

To a solution of compound 1 (50 mg; 0.045 mmol) in ethanol (1.0 mL) were added 2-bromopyrimidine (14 mg; 0.09 mmol) and N,N-diisopropylethylamine (0.15 mL; 0.90 mmol). The resulting mixture was stirred at 80° C. for 22 hours. The reaction mixture was cooled to RT, quenched with water and extracted with ethyl acetate. The organic layers were combined, washed with brine, dried over sodium sulfate, filtered and evaporated to dryness. The residue was purified by reverse phase chromatography (Xbridge prep C18 Sum 19*150 mm; mobile phase A: 10 mmol/L aqueous NH₄HCO₃, mobile phase B: ACN; Flow rate: 25 ml/min; 44% B). The fractions containing the desired product were combined and lyophilized to give 2.7 mg (10%) of compound 192 as a white solid.

### Preparation of Compound 193

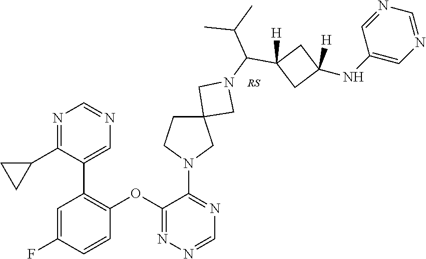 [SMILES: CC(C)C([C@H]1C[C@@H](Nc2cncnc2)C1)N1CC2(CCN(c3ncnnc3Oc3ccc(F)cc3-c3cncnc3C3CC3)C2)C1]

To a solution of compound 1 (300 mg; 0.55 mmol) in toluene (10 mL) were added 5-bromopyrimidine (175 mg; 1.1 mmol), Brettphos (59 mg; 0.11 mmol), BrettPhos-Pd G3 (100 mg; 0.11 mmol) and cesium carbonate (538 mg; 1.65 mmol). The resulting mixture was stirred at 100° C. for 18 hours under nitrogen atmosphere. After cooling to room temperature, the reaction was quenched with water and extracted with ethyl acetate. The organic layers were combined, washed with brine, dried over sodium sulfate, filtered and concentrated under reduced pressure. The residue was purified by reverse phase chromatography (Xbridge prep C18 Sum 19*150 mm; mobile phase A: Water (10 mmol NH₄HCO₃), mobile phase B: ACN, Flow rate: 60 ml/min, gradient from 40% B to 55% B in 7 min). The fractions containing the desired product were combined and lyophilized to give 57.7 mg (17%) of Compound 193 as a white solid.

The compounds in the table below were prepared using an analogous method as described for the preparation of compound 193 starting from the suitable starting materials.

| Compound number | Structure |
|---|---|
| Compound 194 | 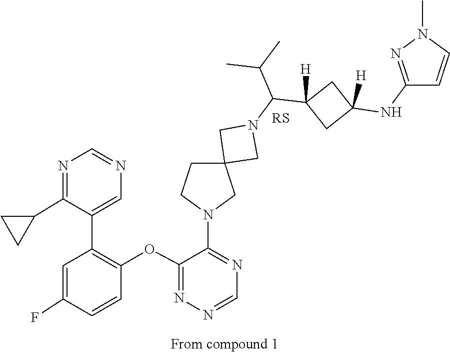 [SMILES: CC(C)C([C@H]1C[C@@H](Nc2ccn(C)n2)C1)N1CC2(CCN(c3ncnnc3Oc3ccc(F)cc3-c3cncnc3C3CC3)C2)C1] |

### Preparation of Compound 370

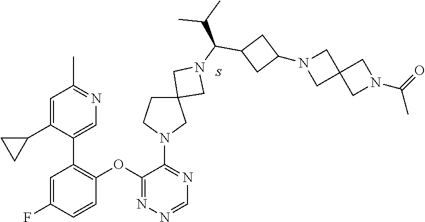

A mixture of intermediate 312 (3.7 g, 14.54 mmol) and 1-(2,6-diazaspiro[3.3]heptan-2-yl)ethan-1-one 2,2,2-trifluoroacetate (3.1 g, 4.85 mmol) in methanol (40 mL) was stirred at room temperature for 30 minutes, and then sodium cyanoborohydride (244 mg, 3.88 mmol) was added into the mixture. After stirring for 1 hour at room temperature, the reaction mixture was quenched with 10% potassium carbonate solution, adjusted to pH=10 with 1 M sodium hydroxide solution and extracted with ethyl acetate. The combined organic layers were dried over anhydrous sodium sulfate. The solid was filtered off. The filtrate was concentrated under reduced pressure. The residue was purified by flash chromatography on silica gel 80 g (eluent: dichloromethane-methanol 93%:7%) to afford 2.1 g (60% yield) of compound 370 as an off white solid containing, based on LCMS, a mixture of cis and trans isomers.

### Preparation of Compound 371 and Compound 372

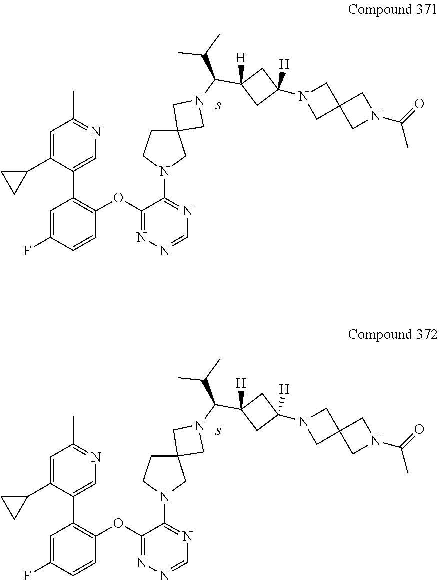

A mixture of cis/trans isomers (2.1 g) was separated by chiral-HPLC with the following conditions: Column: CHIRALPAK IG, 2*25 cm, 5 um; Mobile Phase A: Hex:DCM=3:1 (0.5% 2 M NH₃-MeOH)-Mobile Phase B: EtOH-HPLC; Flow rate: 20 mL/min; Gradient: 15 B to 15 B in 16 min; 254/220 nm; Injection Volume: 0.5 ml; Number of Runs: 9; retention time 1=9.57 min; retention time 2=12.385 min to afford two fractions.

Fraction A: 580.2 mg (36% yield, retention time 1: 9.57 min) of compound 371.

Fraction B: 498.8 mg (30% yield, retention time: 12.385 min) of compound 372.

### Preparation of Compound 373 and Compound 374

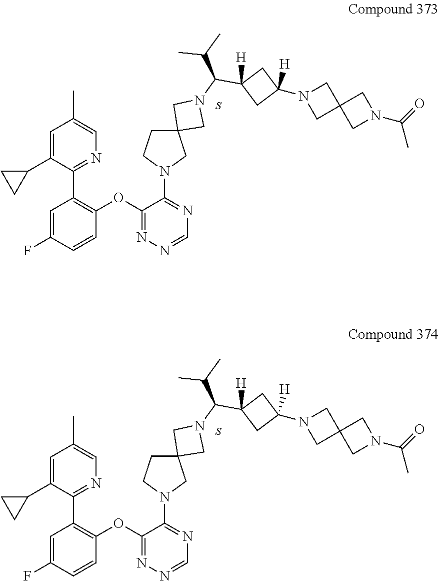

To a solution of intermediate 317 (3.0 g, 5.39 mmol) in methanol (60 mL) was added 1-(2,6-diazaspiro[3.3]heptan-2-yl)ethan-1-one 2,2,2-trifluoroacetate (4.1 g, 8.92 mmol) at room temperature. The resulting mixture was stirred for 1 hour at room temperature. Then, sodium cyanoborohydride (270 mg, 4.31 mmol) was added to the mixture. The reaction mixture was stirred for 2 hours at room temperature. The reaction mixture was quenched with potassium carbonate solution (10% in water) and sodium hydroxide solution (1 M in water) and then, extracted with ethyl acetate for 3 times. The organic layers were combined, washed with brine and dried over sodium sulfate. The solid was filtered off. The filtrate was concentrated under reduced pressure to give a residue. The residue was purified by flash chromatography with silica gel 120 g (eluent: dichloromethane-methanol/0%-10%, 8%) to give 1.78 g of a crude product, which was further purified by high pressure revers phase chromatographic with the following conditions: Welch Ultimate XB-C18 50*250 mm, 10 um; Mobile Phase A: Water (10 mmol/L NH₄HCO₃), Mobile Phase B: ACN; Flow rate: 100 mL/min; Gradient: 30% B to 60% B in 30 min, 220 nm; retention time 1: 17.5 min; retention time 2: 21.5 min to afford two fractions.

Fraction A: 670 mg (18% yield, retention time 1: 17.5 min) of compound 373 as a white solid.

Fraction B: 610 mg (16% yield, retention time 2: 21.5 min) of compound 374 as a white solid.

### Preparation of Compound 375 and Compound 376

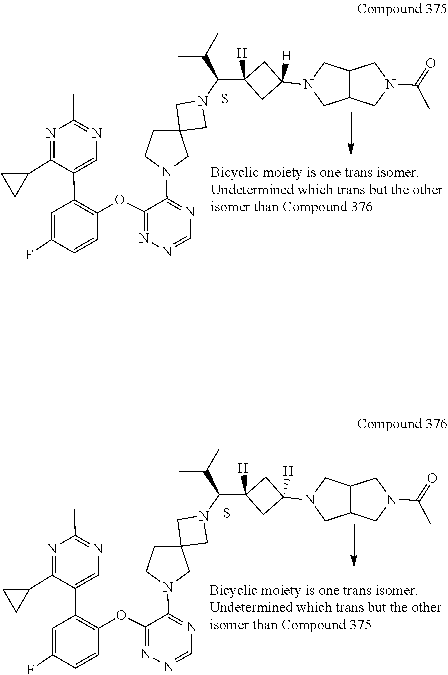

Intermediate 319 (500 mg, crude) was added to a solution consisting of intermediate 324 (600 mg, 1.08 mmol), and MeOH (15 mL). The mixture was stirred at 40° C. for 2 hours. NaBH₃CN (280 mg, 4.46 mmol) was added to the mixture. Then, the mixture was stirred at 40° C. for 2 hours. The mixture was quenched with H₂O (50 mL) and then, extracted with ethyl acetate (50 mL×3). The combined organic layers were dried over anhydrous Na₂SO₄, filtered, and concentrated in vacuo to afford the crude compound which was purified by high performance liquid chromatography over a Phenomenex Gemini-NX 80×40 mm×3 μm column (eluents: A: water (0.05% NH₃H₂O+10 mM NH₄HCO₃)) and B: MeCN; gradient: 34% B to 64% B (v/v) to afford pure product. The product was suspended in water (50 mL). The mixture was frozen using dry ice/ethanol, and then, lyophilized to dryness to afford 300 mg (40% yield) compound 375 and 50 mg (7% yield) of compound 376 as a white solid.

### Preparation of Compound 377 and Compound 378

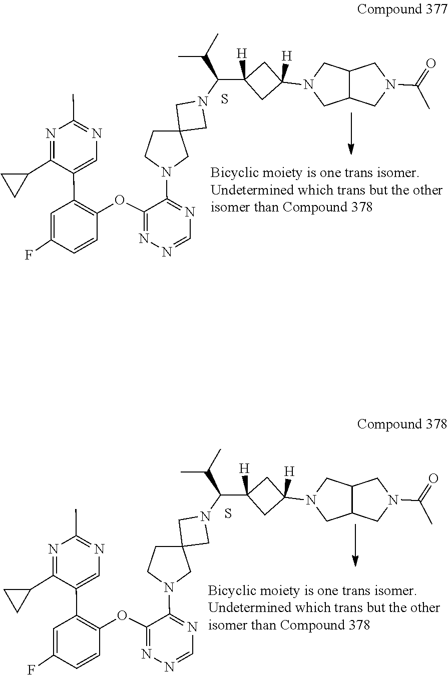

Compound 375 (300 mg, 0.043 mmol) was purified by supercritical fluid chromatography over DAICEL CHIRALPAK AS (250 mm×30 mm×10 um) (Condition: solvent A: supercritical CO₂; solvent B: EtOH (0.1% NH₃·H₂O); A:B=70%: 30%, Flowrate: 80 mL/min). The pure fractions were collected and the volatiles were removed under vacuum. The resulting product was lyophilized to dryness to remove the solvent residue completely. Desired compound 377 (104.9 mg, 97.3% purity, 34% yield) and compound 378 (135.1 mg, 89.4% purity, 40% yield) were obtained as a white solid.

### Compound 377

¹H NMR CHLOROFORM-d (Varian_400 MHz): δ 8.45 (s, 1H), 8.27 (s, 1H), 7.38-7.31 (m, 1H), 7.23-7.15 (m, 1H), 7.13-7.05 (m, 1H), 3.88-3.75 (m, 1H), 3.72-3.40 (m, 5H), 3.22-3.02 (m, 6H), 2.99-2.85 (m, 3H), 2.61 (s, 3H), 2.51-2.41 (m, 2H), 2.39-2.07 (m, 5H), 2.07-1.90 (m, 8H), 1.71-1.62 (m, 2H), 1.16-1.01 (m, 2H), 0.94-0.68 (m, 8H)

### Compound 378

¹H NMR CHLOROFORM-d (Varian_400 MHz): δ 8.43 (s, 1H), 8.26 (s, 1H), 7.39-7.30 (m, 1H), 7.23-7.13 (m, 1H), 7.12-7.04 (m, 1H), 3.92-3.75 (m, 1H), 3.70-3.40 (m, 5H), 3.23-3.09 (m, 6H), 3.07-2.90 (m, 4H), 2.69-2.49 (m, 5H), 2.38-2.14 (m, 4H), 2.08-1.96 (m, 6H), 1.88-1.64 (m, 4H), 1.18-0.99 (m, 2H), 0.95-0.48 (m, 8H)

The compounds listed in the table below were prepared following the same procedure as reported for the preparation of compounds 375 and 376 starting from the corresponding starting materials

| Compound number | Compound structure |
|---|---|
| 379 | 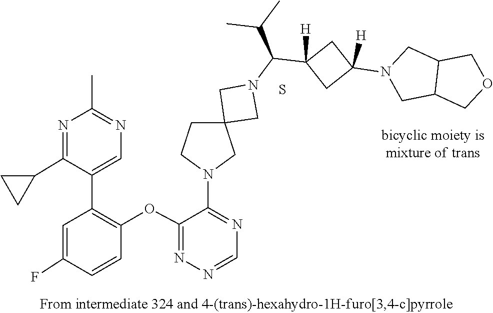 |
| 380 | 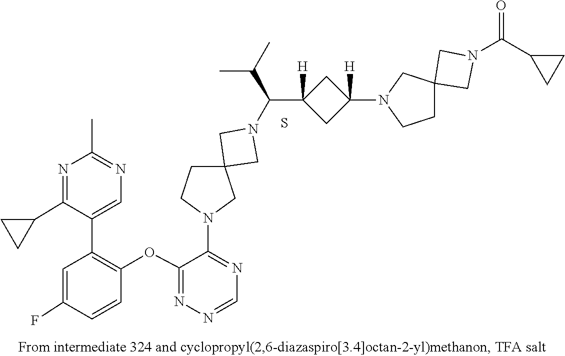 |
| 381 | 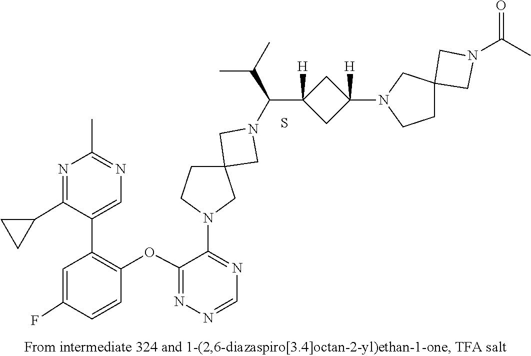 |
| 382 | 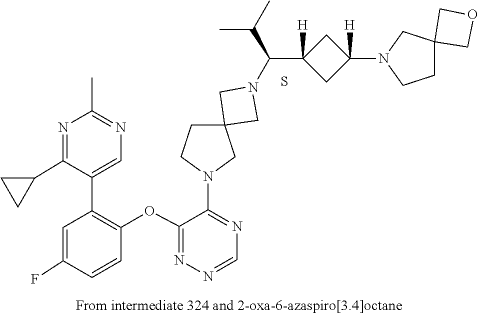 |
| 383 | 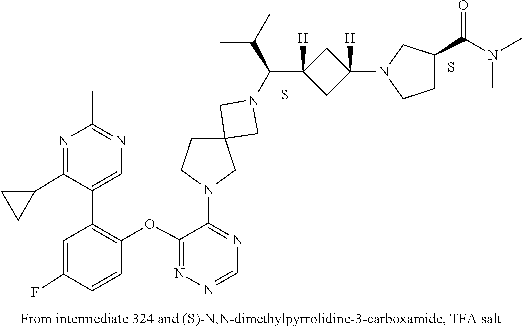 |
| 384 | 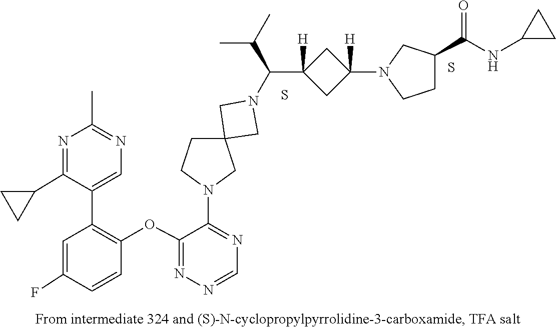 |
| 385 | 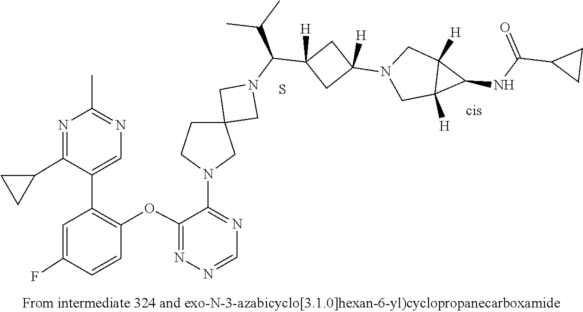 |
| 386 | 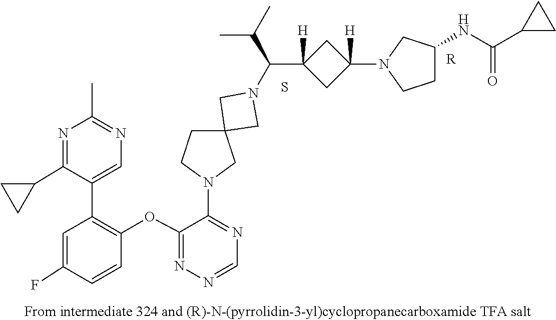 |
| 387 | 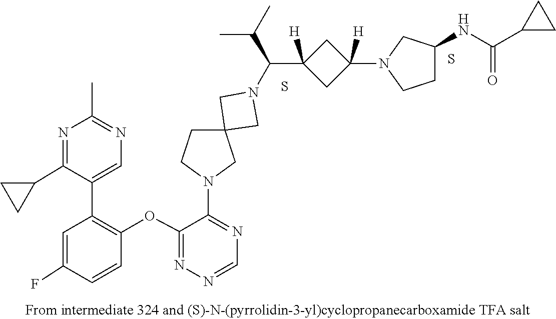 |
| 388 | 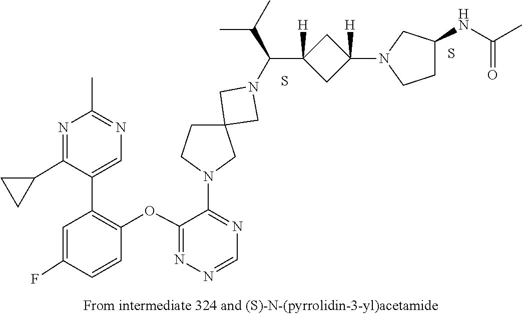 |
| 389 | 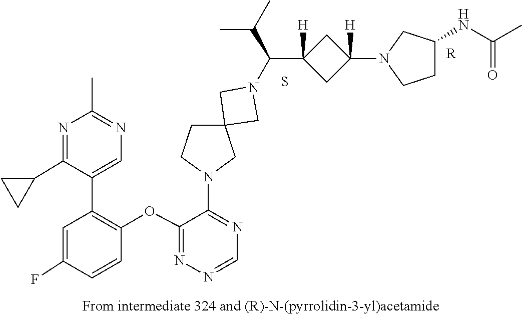 |
| 390 | 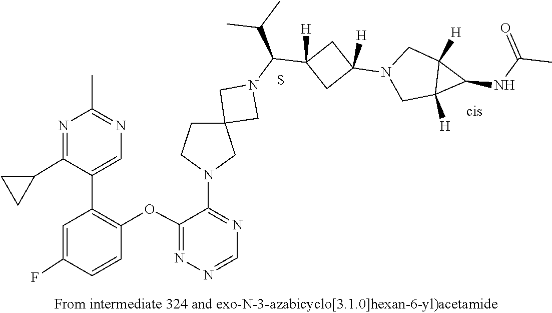 |
| 391 | 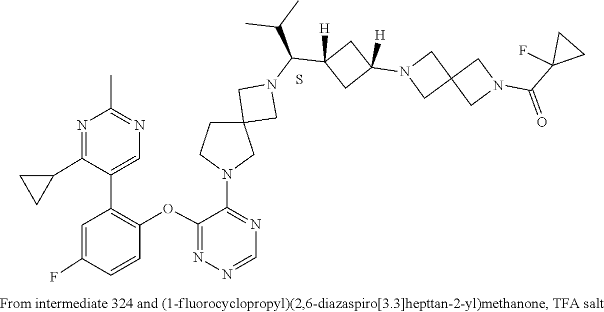 |
| 392 | 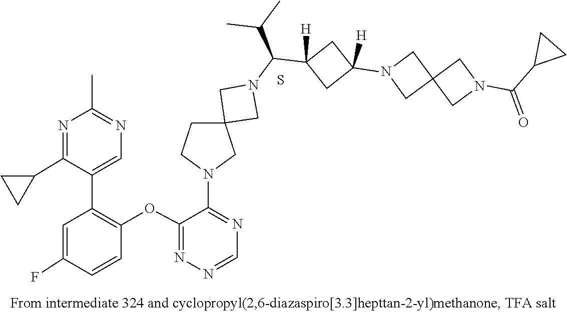 |
| 393 | 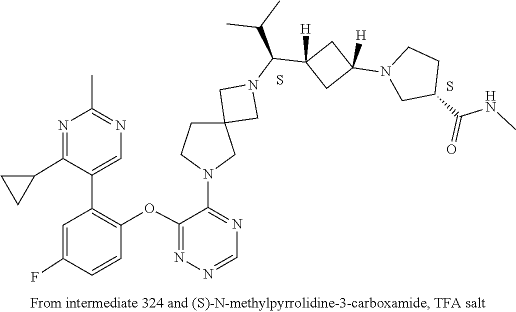 |
| 394 | 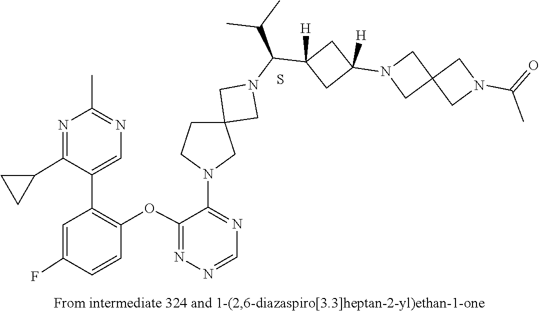 |
| 395 | 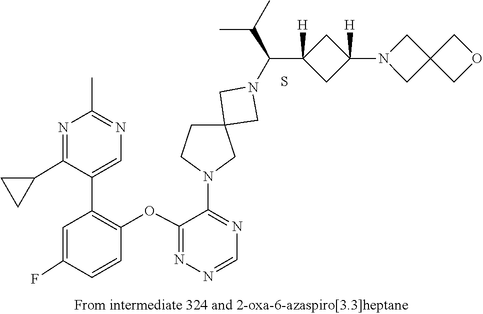 |
| 396 | 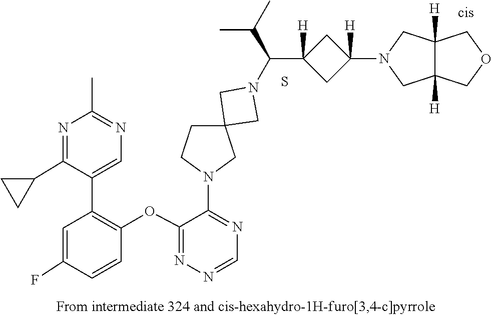 |
| 397 | 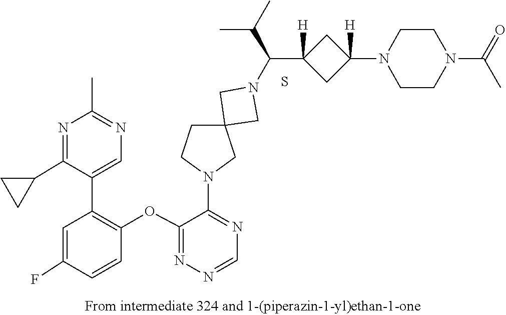 |

### Preparation of Compound 398 and Compound 399

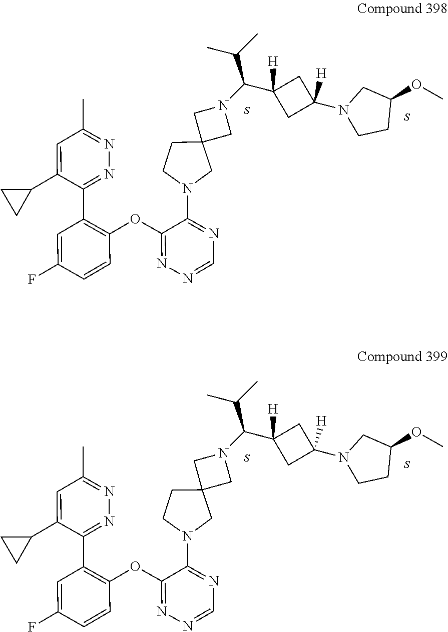

To a stirring solution of intermediate 332 (250 mg, 0.39 mmol) in tetrahydrofuran (5 mL) were added (S)-3-methoxypyrrolidine hydrochloride (106 mg, 0.77 mmol) and acetic acid (0.5 mL). After stirring for 30 minutes at room temperature, sodium triacetoxyborohydride (409 mg, 1.93 mmol) was added. The resulting mixture was stirred at 50° C. for overnight. The reaction mixture were quenched with a solution of potassium carbonate (10% in water) and extracted with dichloromethane. The combined organic layers were dried over anhydrous sodium sulfate, filtered and concentrated under reduced pressure. The crude product was purified by preparative HPLC (Column: XBridge Prep C18 OBD Column, 19×150 mm Sum; Mobile Phase A: Water (10 mmol/L NH₄HCO₃+0.1% NH₃·H₂O), Mobile Phase B: ACN; Flow rate: 25 mL/min; Gradient: 30% B to 50% B in 7 min; 220 nm; retention time 1: 5.47 min; retention time 2:6.57 min. Mixing the pure fractions following by lyophilization gave 65.5 mg (22% yield, retention time 1: 5.47 min) of compound 398 as a white solid and 59.4 mg (19.4% yield, retention time 2: 6.57 min) of compound 399 as a white solid.

| Compound number | Compound structure |
|---|---|
| 400 | 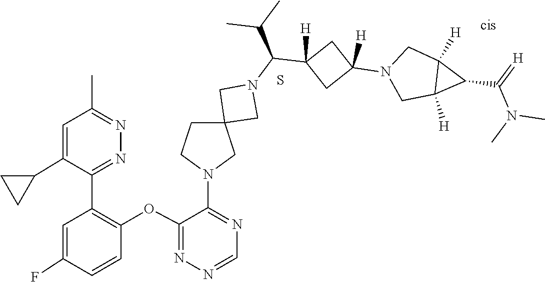 |
| 401 | 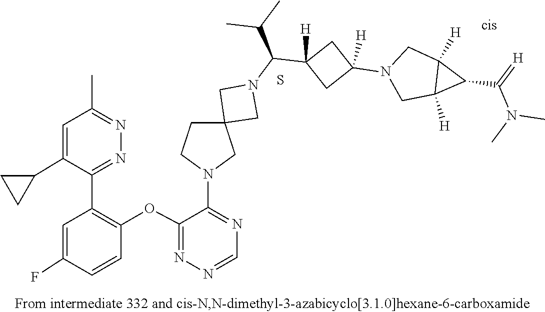 |
| 402 | 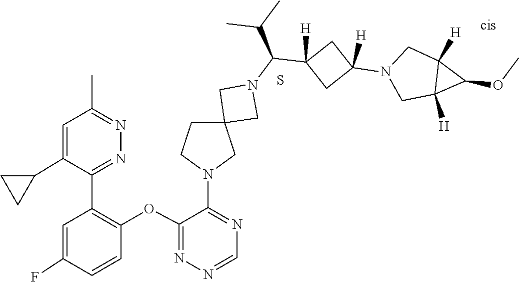 |
| 403 | 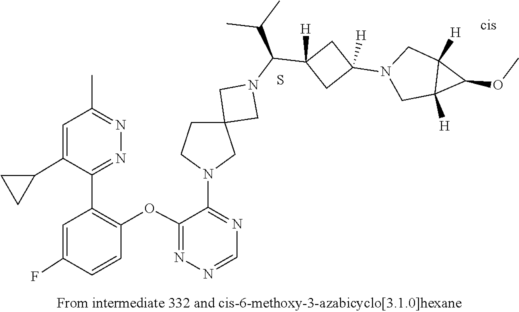 |

### Preparation of Compound 405

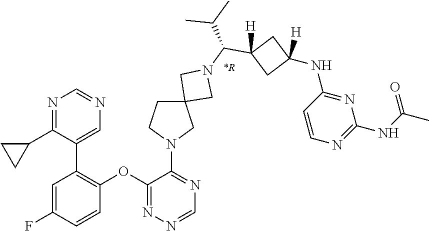 [SMILES: CC(=O)Nc1nccc(N[C@H]2C[C@@H]([C@@H](C(C)C)N3CC4(CCN(c5ncnnc5Oc5ccc(F)cc5-c5cncnc5C5CC5)C4)C3)C2)n1]

Compound 1a (100 mg, 0.184 mmol), N-(4-chloropyrimidin-2-yl)acetamide (60 mg, 0.35 mmol), Cs₂CO₃ (120 mg, 0.368 mmol), KI (6 mg, 0.04 mmol), and DMF (2 mL) were added added to a 10 mL sealed tube. The resultant mixture was stirred at 50° C. for 16 hours. The suspension was filtered through a pad of Celite® and the pad washed with dichloromethane (20 mL). The filtrate was concentrated to dryness under reduced pressure to afford the crude product, which was purified by preparative HPLC (Column: Boston Prime C18 150*30 mm*5 um, Mobile Phase A: water (0.05% NH3H2O)-ACN, Mobile Phase B: acetonitrile, Flow rate: 30 mL/min, gradient condition from 36% B to 66%). The pure fractions were collected and the solvent was evaporated under vacuum to give a residue. The residue was partitioned between acetonitrile (2 mL) and water (5 mL). The solution was lyophilized to dryness to give Compound 405 (21.94 mg, 18%) as a white solid.

### Preparation of Compound 406

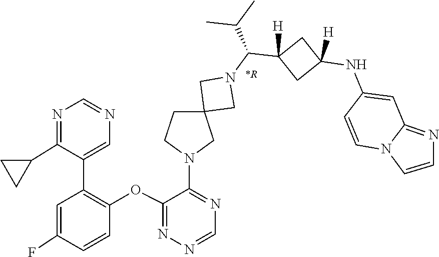 [SMILES: CC(C)[C@H]([C@H]1C[C@@H](Nc2ccn3ccnc3c2)C1)N1CC2(CCN(c3ncnnc3Oc3ccc(F)cc3-c3cncnc3C3CC3)C2)C1]

Ti(OiPr)₄ (700 mg, 2.46 mmol) was added to a solution consisting of intermediate 38b (300 mg, 0.552 mmol), imidazo[1,2-a]pyridin-7-amine (150 mg, 1.13 mmol) and MeOH (1 mL). The mixture was stirred at 80° C. for 6 hours. NaBH₃CN (140 mg, 2.23 mmol) was added to the mixture. Then the mixture was stirred at 80° C. for 5 hours. Aq. NaHCO₃ (20 mL) and water (20 mL) was slowly added to the reaction mixture, and the mixture was extracted with ethyl acetate (50 mL×3). The combined organic layer was dried over Na₂SO₄, filtered and concentrated to dryness under reduced pressure to afford the crude product which was purified by high performance liquid chromatography over a Boston Green ODS 150×30 mm×5 μm column (eluent: 10% to 40% (v/v) CH3CN and H2O with 0.225% TFA) to afford pure product. The product was suspended in water (10 mL), the mixture frozen using dry ice/ethanol, and then lyophilized to dryness to afford compound (38 mg) as a white solid which was purified by supercritical fluid chromatography over DAICEL CHIRALPAK AD (250 mm×30 mm×10 um) (eluent: supercritical CO₂ in EtOH (0.1% v/v ammonia) 50/50, v/v). The pure fractions were collected and the volatiles were removed under vacuum. The resulting product was lyophilized to dryness to remove the solvent residue completely to give Compound 406 (4.65 mg, 1% yield) as a white solid.

### Preparation of Compound 407

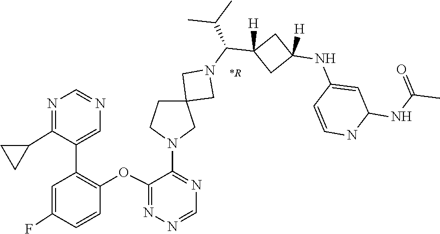 [SMILES: CC(=O)NC1C=C(N[C@H]2C[C@@H]([C@@H](C(C)C)N3CC4(CCN(c5ncnnc5Oc5ccc(F)cc5-c5cncnc5C5CC5)C4)C3)C2)C=CN1]

A solution consisting of N-(4-fluoropyridin-2-yl)acetamide (100 mg, 0.649 mmol), Compound 1a (20 mg, 0.037 mmol), K₂CO₃ (20.3 mg, 0.147 mmol) and DMF (2 mL) was stirred at 120° C. overnight. The crude material was submitted to prep. HPLC for purification and the collected fraction was lyophilized to dryness to afford Compound 407 (5.15 mg, 19.4% yield) as a white powder.

### Preparation of Compound 408

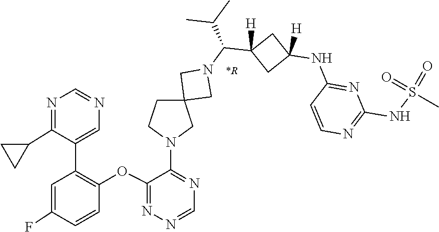 [SMILES: CC(C)[C@H]([C@H]1C[C@@H](Nc2ccnc(NS(C)(=O)=O)n2)C1)N1CC2(CCN(c3ncnnc3Oc3ccc(F)cc3-c3cncnc3C3CC3)C2)C1]

To a solution of N-(4-chloropyrimidin-2-yl)methanesulfonamide (52.6 mg, 0.253 mmol) and Compound 1a (115 mg, 0.211 mmol) in EtOH (3 mL) was added TEA (64.1 mg, 0.633 mmol). The mixture was stirred at 90° C. for 16 hours. The reaction mixture was directly purified by pre-HPLC (Column: Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 33% B to 63% B). The pure fractions were collected and evaporated to give the product as a white solid (99 mg, crude). And then the crude product was further purified by pre-HPLC (Column: Boston Green ODS 150*30 mm*5 um, Mobile Phase A: water (0.225% FA), Mobile Phase B: acetonitrile, Flow rate: 35 mL/min, gradient condition from 10% B to 40% B). The pure fractions were collected and evaporated to give Compound 408 (53.77 mg, 33% yield) as a white solid.

### Preparation of Compound 409

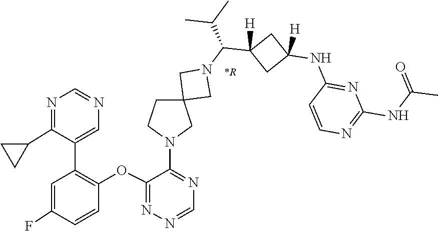 [SMILES: CC(=O)Nc1nccc(N[C@H]2C[C@@H]([C@@H](C(C)C)N3CC4(CCN(c5ncnnc5Oc5ccc(F)cc5-c5cncnc5C5CC5)C4)C3)C2)n1]

Compound 1a (200 mg, 0.367 mmol), N-(6-bromopyridin-2-yl)acetamide (160 mg, 0.774 mmol), Cs₂CO₃ (360 mg, 1.11 mmol) were dissolved in dioxane (10 mL). The resultant mixture was sparged with Ar for 2 minutes and then treated with Brettphos-Pd-G3 (20.0 mg, 0.022 mmol). The resultant mixture was sparged with Ar for another 2 minutes and then stirred at 90° C. overnight. The reaction mixture was filtered and concentrated under reduced pressure to give a residue. The residue was first purified by prep-HPLC (Column: Welch Xtimate C18 150*25 mm*5 um, Mobile Phase A: water (0.2% FA), Mobile Phase B: acetonitrile, Flow rate: 25 mL/min, gradient condition from 10% B to 40%) and then by prep-HPLC (Column: Phenomenex Luna C18 150*30 mm*5 um, Mobile Phase A: water (0.05% NH₃H₂O), Mobile Phase B: acetonitrile, Flow rate: 30 mL/min, gradient condition from 43% B to 73%). The pure fractions were collected and lyophilized to afford Compound 409 (30.55 mg, 12% yield) as a white powder.

The compounds listed in the table below were prepared following the same procedure as reported for the preparation of Compound 409 starting from the corresponding starting materials

| Compound number | Compound structure |
|---|---|
| 410 | 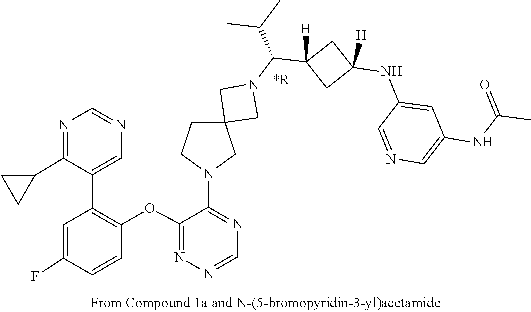 |
| 414 | 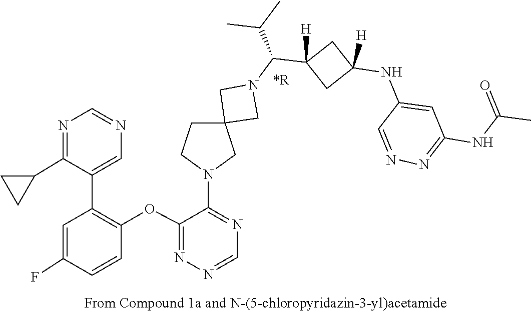 |

### Preparation of Compound 411

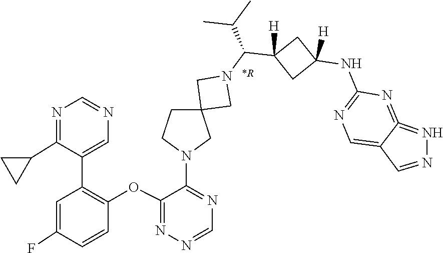

A stir bar, Compound 1a (120 mg, 0.220 mmol), 6-chloro-1H-pyrazolo[3,4-d]pyrimidine (35.0 mg, 0.26 mmol), N,N-diisopropylethylamine (57.0 mg, 0.441 mmol) and acetonitrile (3 mL) were taken up into a microwave tube. The sealed tube was heated at 90° C. for 1 h under microwave. The mixture was diluted into dichloromethane (30 mL) and washed with water (10 mL×3). The organic layer was dried over anhydrous Na₂SO₄, filtered and concentrated under reduced pressure to give the crude which was purified by prep. HPLC (Column: Phenomenex Gemini-NX 80*40 mm*3 um, Mobile Phase A: water (0.05% NH₃H₂O), Mobile Phase B: acetonitrile, Flow rate: 30 mL/min, gradient condition from 33% B to 63%). The pure fractions were collected and the solvent was evaporated under vacuum to give a residue. The residue was partitioned between acetonitrile (2 mL) and water (10 mL). The solution was lyophilized to dryness to give Compound 411 (78.88 mg, 53.9% yield) as a white powder.

### Preparation of Compound 412

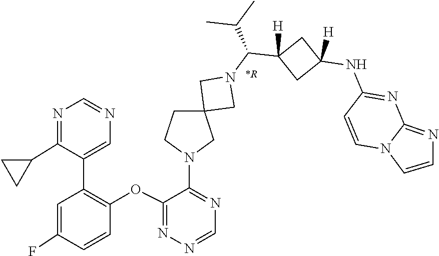 [SMILES: CC(C)[C@H]([C@H]1C[C@@H](Nc2ccn3ccnc3n2)C1)N1CC2(CCN(c3ncnnc3Oc3ccc(F)cc3-c3cncnc3C3CC3)C2)C1]

To the solution of intermediate 333 (58.0 mg, 0.910 mmol) in ethanol (2 mL) and H₂O (0.35 mL), was added 2-chloroacetaldehyde (35.7 mg, 0.182 mmol) and NaHCO₃ (11.5 mg, 0.136 mmol). The mixture was stirred at 70° C. overnight. The mixture was purified by SFC over DAICEL CHIRALPAK IG (250 mm*30 mm, 10 um) (eluent: 60% to 60% (v/v) supercritical 0.1% NH₃H₂O ETOH). The pure fractions were collected and the volatiles were removed under reduced pressure. The product was suspended in water (10 mL), the mixture frozen using dry ice/acetone, and then lyophilized to dryness to afford the crude material which was further purified by SFC over DAICEL CHIRALPAK AD (250 mm*30 mm, 10 um) (eluent: 50% to 50% (v/v) supercritical 0.1% NH₃H₂O ETOH). The pure fractions were collected and the volatiles were removed under reduced pressure. The product was suspended in water (10 mL), the mixture frozen using dry ice/acetone, and then lyophilized to dryness to afford Compound 412 (7.1 mg, 11.7% yield) as a white solid.

### Preparation of Compound 413

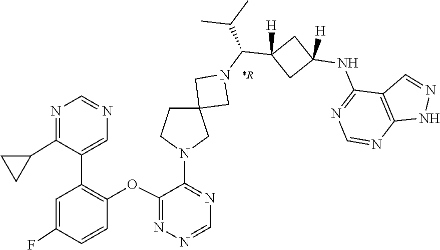 [SMILES: CC(C)[C@H]([C@H]1C[C@@H](Nc2ncnc3[nH]ncc23)C1)N1CC2(CCN(c3ncnnc3Oc3ccc(F)cc3-c3cncnc3C3CC3)C2)C1]

A stirbar, Compound 1a (120 mg, 0.220 mmol), 4-chloro-1H-pyrazolo[3,4-d]pyrimidine (34.2 mg, 0.221 mmol), N-ethyl-N-isopropylpropan-2-amine (57.0 mg, 0.441 mmol) and acetonitrile (3 mL) were taken up into a microwave tube. The sealed tube was heated at 90° C. for 1 h under microwave. The mixture was cooled to room temperature, then the mixture was concentrated under reduced pressure to give a residue which was suspended into water (50 mL) and extracted with dichloromethane (30 mL×3). The organic layers were dried over anhydrous Na₂SO₄, filtered and concentrated under reduced pressure to give a crude, which was purified by prep. HPLC (Column: Phenomenex Gemini-NX 80*40 mm*3 um, Mobile Phase A: water (0.05% NH₃H₂O), Mobile Phase B: acetonitrile, Flow rate: 30 mL/min, gradient condition from 28% B to 58%). The pure fractions were collected and the solvent was evaporated under vacuum to give a residue. The residue was partitioned between acetonitrile (2 mL) and water (10 mL). The solution was lyophilized to dryness to give Compound 413 as a white powder (51.8 mg, 35.5% yield).

### Preparation of Compound 415

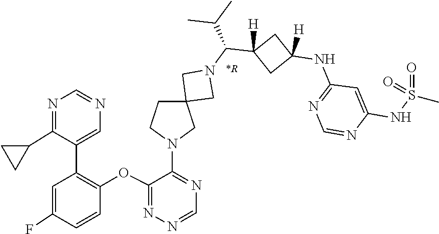 [SMILES: CC(C)[C@H]([C@H]1C[C@@H](Nc2cc(NS(C)(=O)=O)ncn2)C1)N1CC2(CCN(c3ncnnc3Oc3ccc(F)cc3-c3cncnc3C3CC3)C2)C1]

DIPEA (0.03 mL, 0.184 mmol) was added to a solution consisting of Compound 1a (50 mg, 0.092 mmol), N-(4-fluoropyridin-2-yl)methanesulfonamide (21 mg, 0.11 mmol) and i-PrOH (1 mL). The mixture was stirred at 95° C. for 2 hours. The mixture was concentrated under reduced pressure to give crude product, which was purified by preparative HPLC using a Boston Prime C18 150*30 mm*5 um column (eluent: 35% to 65% (v/v) CH₃CN and H₂O with 0.05% NH₃) to afford the pure product. The pure fractions were collected and lyophilized to dryness to remove the solvent residue completely to yield Compound 415 (42 mg, 63.2% yield) as a white solid.

### Preparation of Compound 416

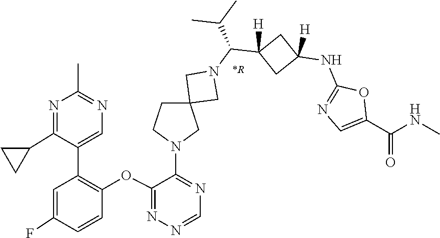 [SMILES: CNC(=O)c1cnc(N[C@H]2C[C@@H]([C@@H](C(C)C)N3CC4(CCN(c5ncnnc5Oc5ccc(F)cc5-c5cnc(C)nc5C5CC5)C4)C3)C2)o1]

A stir bar, Compound 466 (120 mg, 0.179 mmol), methanamine hydrochloride (60.5 mg, 0.896 mmol), TEA (0.125 mL, 0.896 mmol) in DCM (3 mL) was added T3P (171 mg, 0.269 mmol). The mixture was stirred at 35° C. for 10 h. The mixture was purified by prep-HPLC (Column: Phenomenex Gemini-NX 150*30 mm*5 um, Mobile Phase A: water (0.05% NH₃H₂O), Mobile Phase B: acetonitrile, Flow rate: 30 mL/min, gradient condition from 34% B to 64%) to give Compound 416 (16.97 mg, 13.5% yield) as a white solids.

The compounds listed in the table below were prepared following the same procedure as reported for the preparation of Compound 416 starting from the corresponding starting materials

| Compound number | Compound structure |
|---|---|
| 417 | 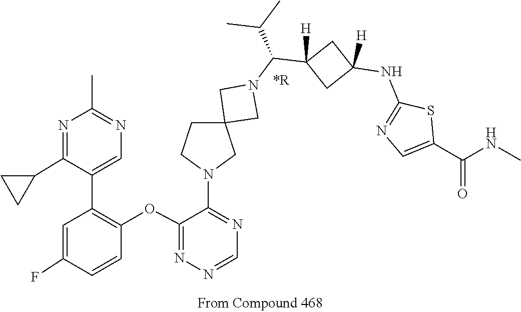 |

### Preparation of Compound 418

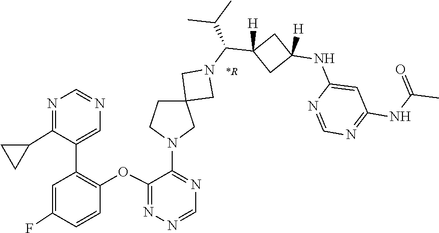 [SMILES: CC(=O)Nc1cc(N[C@H]2C[C@@H]([C@@H](C(C)C)N3CC4(CCN(c5ncnnc5Oc5ccc(F)cc5-c5cncnc5C5CC5)C4)C3)C2)ncn1]

N-(6-bromopyrimidin-4-yl)acetamide (72 mg, 0.33 mmol) was added to a solution consisting of Compound 1a (120 mg, 0.220 mmol), TEA (0.1 mL, 0.72 mmol), and tBuOH (5 mL). The mixture was stirred at 120° C. for 16 hours. The mixture was quenched with solution of H₂O (5 mL), and then extracted with EA (10 mL×3). The combined organic layers were dried over anhydrous Na₂SO₄, filtered, and concentrated in vacuo to give the crude product was purified by preparative HPLC using Phenomenex Gemini-NX C18 75×30 mm×3 μm column (eluent: 40% to 70% (v/v) water (0.05% NH₃H₂O+10 mM NH₄HCO₃)-ACN) to afford pure product. The product was suspended in water (10 mL), and the mixture frozen using dry ice/ethanol, and then lyophilized to dryness to afford Compound 418 (41.34 mg, 27.53% yield) as a white solid.

### Preparation of Compound 419

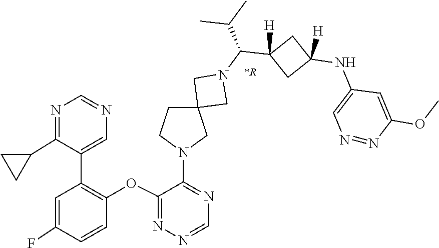 [SMILES: COc1cc(N[C@H]2C[C@@H]([C@@H](C(C)C)N3CC4(CCN(c5ncnnc5Oc5ccc(F)cc5-c5cncnc5C5CC5)C4)C3)C2)cnn1]

tBuXPhos Pd G3 (7.3 mg, 0.009 mmol) was added to a solution of Compound 1a (50 mg, 0.092 mmol), 5-bromo-3-methoxypyridazine (33 mg, 0.175 mmol) and NaOtBu (26.4 mg, 0.275 mmol) in 1,4-dioxane (8 mL) under argon atmosphere. The mixture was stirred at 100° C. under microwave for 1 h. The reaction was repeated at the same scale and the combined reaction mixture was cooled to room temperature and concentrated under reduced pressure to give crude product, which was diluted with DCM (20 mL) and was washed with H₂O (10 mL), brine (10 mL), dried over Na₂SO₄, filtered and concentrated under reduced pressure to give crude product, which was purified by preparative HPLC (Column: Phenomenex Gemini-NX C18 75*30 mm*3 um, Mobile Phase A: water (0.225% FA), Mobile Phase B: acetonitrile, Flow rate: 30 mL/min, gradient condition from 0% B to 25%). The pure fraction was collected and the solvent was evaporated under vacuum to give a residue, The residue was diluted with H₂O (3 mL), adjusted to pH=8 by the saturated solution of sodium bicarbonate. Then the resultant mixture was extracted with CH₂Cl₂ (10 mL×2), dried over Na₂SO₄, filtered and concentrated under reduced pressure to give the product (60 mg, 93.46% purity, 46.79% yield) which was further purified by SFC (column: DAICEL CHIRALPAK AS (250 mm*30 mm, 10 um), eluent: 35% (v/v) super critical CO₂ in 0.1% NH₃H₂O ETOH, flow rate: 70 mL/min), The pure fractions were collected and the volatile solvent was evaporated under vacuum to give the residue which was lyophilized to give Compound 419 (25.44 mg, purity 99.43%, 42.16% yield) as a white powder.

The compounds listed in the table below were prepared following the same procedure as reported for the preparation of Compound 419 starting from the corresponding starting materials

| Compound number | Compound structure |
|---|---|
| 421 | 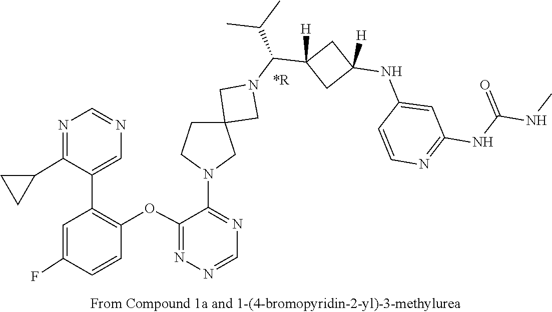 |
| 426 | 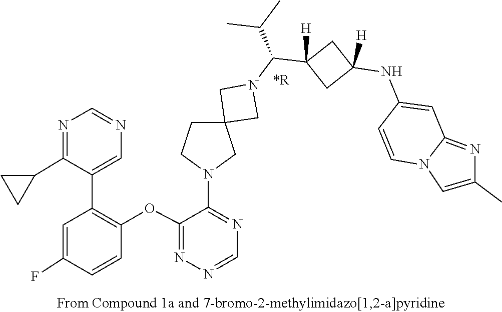 |
| 428 | 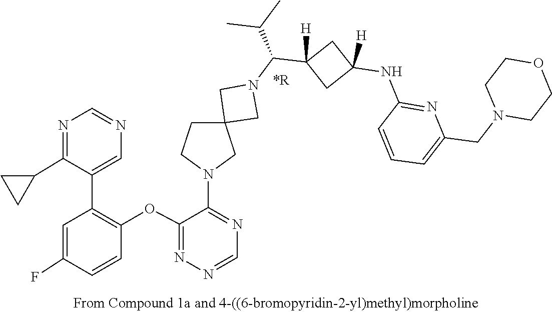 |

### Preparation of Compound 420

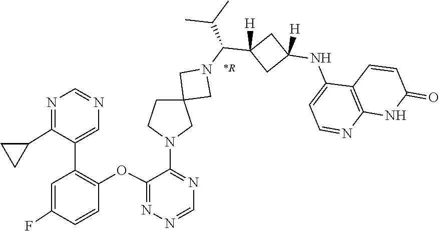 [SMILES: CC(C)[C@H]([C@H]1C[C@@H](Nc2ccnc3[nH]c(=O)ccc23)C1)N1CC2(CCN(c3ncnnc3Oc3ccc(F)cc3-c3cncnc3C3CC3)C2)C1]

A solution of Intermediate 339 (150 mg, 0.185 mmol) in TFA (5 mL) was stirred at 75° C. for 2 h. The mixture was concentrated under reduced pressure and diluted with CH₂Cl₂ (10 mL), adjusted to pH=13 with NaOH (2 M). The resultant mixture was extracted with CH₂Cl₂ (10 mL×2). The combined organic extracts were washed with brine (20 mL), dried over Na₂SO₄, filtered, and concentrated to dryness under reduced pressure to give crude product, which was purified by preparative HPLC (Column: Phenomenex Gemini-NX 80*40 mm*3 um, Mobile Phase A: water (0.05% NH₃H₂O), Mobile Phase B: acetonitrile, Flow rate: 30 mL/min, gradient condition from 30% B to 60%). The pure fractions were collected and the volatile solvent was evaporated under vacuum. The resultant aqueous mixture was lyophilized to dryness to give the product (25 mg, purity 99.99%, 19.57% yield) as a white powder (F-NMR showed TFA residual). The product was diluted with CH₂Cl₂ (15 mL), washed with 2 M NaOH (5 mL), dried over Na₂SO₄, filtered, and concentrated to dryness under reduced pressure to give a residue. The residue was partitioned between acetonitrile (3 mL) and water (10 mL). The solution was lyophilized to dryness to give Compound 420 (8.86 mg, 6.84% yield) as a white powder.

### Preparation of Compound 422

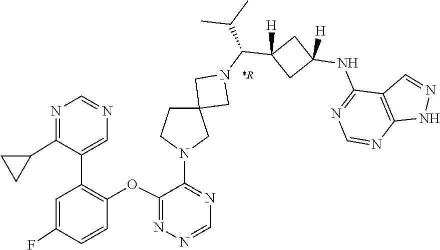 [SMILES: CC(C)[C@H]([C@H]1C[C@@H](Nc2ncnc3[nH]ncc23)C1)N1CC2(CCN(c3ncnnc3Oc3ccc(F)cc3-c3cncnc3C3CC3)C2)C1]

To a solution of Compound 470 (45.0 mg, 0.059 mmol) in anhydrous dichloromethane (2 mL) was added trifluoroacetic acid (2 mL). The reaction mixture was stirred at 25° C. for 1 hour. The reaction mixture was concentrated under reduced pressure to give a residue, which was purified by preparative-HPLC over (Column: Boston Prime C18 150*30 mm*5 um, Mobile Phase A: water (0.05% NH₃H₂O+10 mM NH₄HCO₃), Mobile Phase B: acetonitrile, Flow rate: 25 mL/min, gradient condition from 45% B to 75%). The pure fractions were collected and the solvent was evaporated under vacuum. The residue was partitioned between acetonitrile (2 mL) and water (10 mL). The mixture was lyophilized to dryness to give Compound 422 (10.73 mg, 96.84% purity, 26.6% yield) as a white powder.

### Preparation of Compound 423

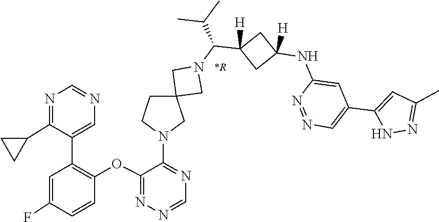 [SMILES: Cc1cc(-c2cnnc(N[C@H]3C[C@@H]([C@@H](C(C)C)N4CC5(CCN(c6ncnnc6Oc6ccc(F)cc6-c6cncnc6C6CC6)C5)C4)C3)c2)[nH]n1]

3-chloro-5-(3-methyl-1H-pyrazol-5-yl)pyridazine (100 mg, 0.514 mmol), Compound 1a (140 mg, 0.257 mmol), NaOtBu (75 mg, 0.78 mmol) and T-Amyl-OH (5 mL) were added to a 8 mL reaction flask. The resultant mixture was sparged with N₂ for 5 minutes and then treated with t-BuXPhos-Pd-G3 (20 mg, 0.025 mmol). The resultant mixture was sparged with N₂ for another 5 minutes and then the resultant mixture was heated to 130° C. for 12 hours before cooling to room-temperature. The resultant mixture was concentrated to dryness under reduced pressure to afford the crude product, which was purified by preparative HPLC using a Phenomenex Gemini-NX C18 75*30 mm*3 um Column (eluent: 40% to 70% (v/v) CH₃CN and water (0.05% NH₃H₂O+10 mM NH₄HCO₃)) to afford product. which was purified by preparative HPLC using a Boston Prime C18 150*30 mm*5 um Column (eluent: 40% to 70% (v/v) CH₃CN and water (0.05% NH₃H₂O+10 mM NH₄HCO₃)) to afford product. The product was suspended in water (10 mL), the mixture frozen using dry ice/acetone, and then lyophilized to dryness to afford Compound 423 (8.20 mg, 5%) as a white solid.

The compounds listed in the table below were prepared following the same procedure as reported for the preparation of Compound 423 starting from the corresponding starting materials

| Compound number | Compound structure |
|---|---|
| 425 | 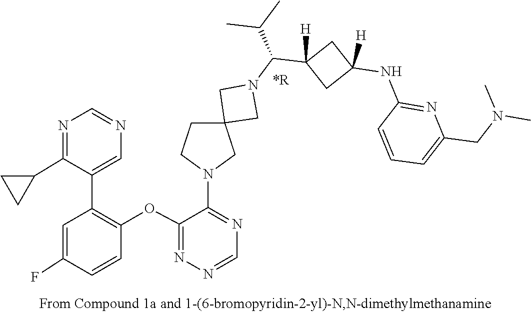 |

### Preparation of Compound 424

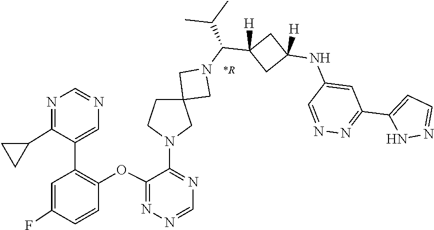 [SMILES: CC(C)[C@H]([C@H]1C[C@@H](Nc2cnnc(-c3ccn[nH]3)c2)C1)N1CC2(CCN(c3ncnnc3Oc3ccc(F)cc3-c3cncnc3C3CC3)C2)C1]

(1H-pyrazol-5-yl)boronic acid (15.3 mg, 0.137 mmol), Compound 471 (45 mg, 0.068 mmol) and K₃PO₄ (44 mg, 0.21 mmol) were added to a 10 mL microwave tube and the resulting mixture dissolved in 1,4-dioxane (4 mL) and H₂O (0.5 mL). The resultant mixture was sparged with Ar for 5 minutes and then treated with pd-peppsi(tm)-ipent catalyst (5.5 mg, 0.0070 mmol). The resultant mixture was stirred at 100° C. for 12 hours before cooling to room-temperature. The mixture was purified by preparative high performance liquid chromatography over Phenomenex Gemini-NX C18 75*30 mm*3 um (eluent: 40% to 70% (v/v) CH₃CN and water (0.05% NH₃H₂O+10 mM NH₄HCO₃)-ACN) to afford pure product. The pure fractions were collected and lyophilized to dryness to afford Compound 424 (5.27 mg, 11.27%).

### Preparation of Compound 427

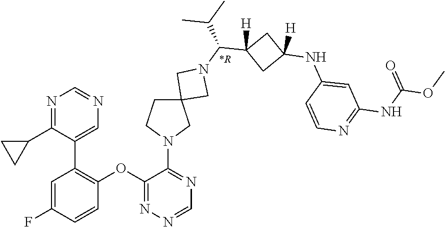 [SMILES: COC(=O)Nc1cc(N[C@H]2C[C@@H]([C@@H](C(C)C)N3CC4(CCN(c5ncnnc5Oc5ccc(F)cc5-c5cncnc5C5CC5)C4)C3)C2)ccn1]

To a solution of Compound 472 (175 mg, 0.267 mmol) in 1,4-dioxane (3 ml) was added methyl carbamate (87 mg, 1.16 mmol), t-BuONa (88 mg, 0.916 mmol) and tBuXPhos-Pd-G3 (17 mg, 0.021 mmol) with Ar₂ in a microwave apparatus. The mixture was stirred at 110° C. for 6 hours. The mixture was filtered and the filtrate was concentrated under reduced pressure. The compound was further purified by preparative HPLC using a Boston Prime C18 150*30 mm*5 um (eluent: 50% to 80% (v/v) CH₃CN and water (0.05% NH₃H₂O)-ACN) to afford pure product. The product was lyophilized to dryness to afford Compound 427 (9 mg, 5% yield) as a white solid.

### Preparation of Compound 429

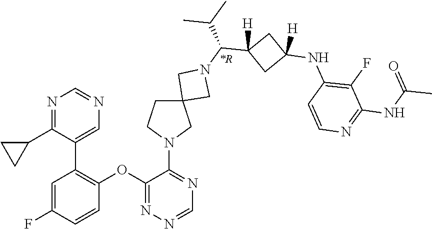 [SMILES: CC(=O)Nc1nccc(N[C@H]2C[C@@H]([C@@H](C(C)C)N3CC4(CCN(c5ncnnc5Oc5ccc(F)cc5-c5cncnc5C5CC5)C4)C3)C2)c1F]

To a suspension of Compound 1a (200 mg, 0.367 mmol) in 1,4-dioxane (3 ml) was added N-(3-fluoro-4-iodopyridin-2-yl)acetamide (150 mg, 0.536 mmol), Cs₂CO₃ (390 mg, 1.20 mmol) and BrettPhos-Pd-G3 (20 mg, 0.022 mmol) with Ar in a microwave apparatus. The mixture was stirred at 100° C. for 4 hours. The mixture was filtered and the filtrate was concentrated to give a crude product, which was further purified by preparative HPLC using a Boston Prime C18 150*30 mm*5 um (eluent: 44% to 74% (v/v) CH₃CN and water (0.05% NH₃H₂O)-ACN) to afford pure product. The product was lyophilized to dryness to afford Compound 429 (37.11 mg, 14% Yield) as a white solid.

### Preparation of Compound 430

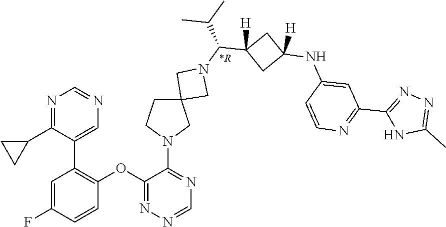 [SMILES: Cc1nnc(-c2cc(N[C@H]3C[C@@H]([C@@H](C(C)C)N4CC5(CCN(c6ncnnc6Oc6ccc(F)cc6-c6cncnc6C6CC6)C5)C4)C3)ccn2)[nH]1]

To a suspension of Compound 473 (100 mg, 0.155 mmol) in DMSO (4 ml) was added acetimidamide hydrochloride (36 mg, 0.343 mmol, 90% purity), Cs₂CO₃ (152 mg, 0.467 mmol) and CuBr (2.0 mg, 0.014 mmol). The mixture was stirred at 120° C. for 2.5 hours. The mixture was filtered and the filtrate was concentrated to give a crude product. The crude product was diluted with DCM (15 ml) and washed with H₂O (10 ml×3). The combined organic layers were washed with brine (15 ml), dried over Na₂SO₄, filtered and concentrated to give a crude product. The crude product was purified by FCC (DCM:MeOH=10:1) to afford the crude product, which was further purified by preparative HPLC using a Phenomenex Gemini-NX C18 75*30 mm*3 um (eluent: 0% to 30% (v/v) CH₃CN and water (0.225% FA)-ACN) to afford pure product. The product was lyophilized to dryness to afford the product (53 mg, crude) as a green solid. HNMR showed the peaks don't split well due to Cu residue. To a solution of the material (53 mg, 0.075 mmol) in MeCN (0.5 mL) and MeOH (0.1 mL) was added NH₃·H₂O (0.5 mL). The resultant mixture was stirred at r.t. for 3 hours. The resultant mixture was purified by preparative HPLC using a Phenomenex Gemini-NX 80*40 mm*3 um (eluent: 32% to 62% (v/v) CH₃CN and water (0.05% NH₃H₂O)-ACN) to afford pure product. The product was lyophilized to dryness to afford Compound 430 (22 mg, 41% yield) as a white solid.

### Preparation of Compound 431

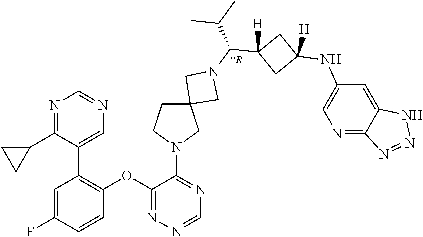 [SMILES: CC(C)[C@H]([C@H]1C[C@@H](Nc2cnc3nn[nH]c3c2)C1)N1CC2(CCN(c3ncnnc3Oc3ccc(F)cc3-c3cncnc3C3CC3)C2)C1]

To a solution of Compound 474 (174 mg, 0.219 mmol) in DCM (5 mL) was added TFA (12 mL). The resultant mixture was stirred at r.t. for 2 hours. The resultant mixture was concentrated under reduced pressure to give a product. The crude product was dissolved in MeCN (1 mL). NH₃·H₂O (0.5 mL) was added and stirred at r.t. for 15 min. Then the mixture was purified by preparative HPLC using a Phenomenex Gemini-NX 80*40 mm*3 um (eluent: 15% to 45% (v/v) CH₃CN and water (0.05% NH₃H₂O)-ACN) to afford pure product. The product was lyophilized to dryness to afford Compound 431 (61 mg, 41% yield) as a white solid.

### Preparation of Compound 432

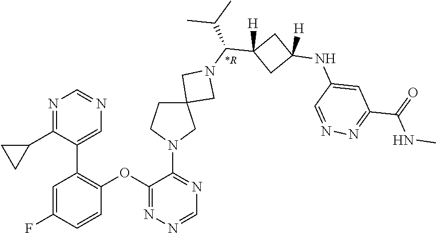 [SMILES: CNC(=O)c1cc(N[C@H]2C[C@@H]([C@@H](C(C)C)N3CC4(CCN(c5ncnnc5Oc5ccc(F)cc5-c5cncnc5C5CC5)C4)C3)C2)cnn1]

To a mixture of Compound 475 (55.0 mg, 0.081 mmol) and K₂CO₃ (22.0 mg, 0.159 mmol) in THE (1 mL) was added methanamine in EtOH (25.0 mg, 0.241 mmol, 30% w/t). The mixture was stirred for 6 h at rt. The reaction mixture was diluted with DCM (15 mL), washed with brine (5 mL), dried over Na₂SO₄. After filtration and concentration, the crude product was purified by FCC (DCM:MeOH=10:1) to give desired product (16 mg, crude) which was further purified by pre-HPLC (Conditions: Column: Welch Xtimate C18 150*25 mm*5 um, Mobile Phase A: water (0.2% FA), Mobile Phase B: acetonitrile, Flow rate: 25 mL/min, gradient condition from 8% B to 38%). The pure fractions were collected and lyophilized to afford Compound 432 (2.82 mg, 4.8% yield) as a white solid.

### Preparation of Compound 433

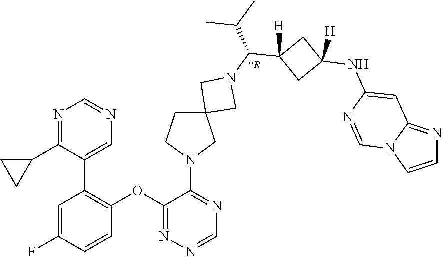 [SMILES: CC(C)[C@H]([C@H]1C[C@@H](Nc2cc3nccn3cn2)C1)N1CC2(CCN(c3ncnnc3Oc3ccc(F)cc3-c3cncnc3C3CC3)C2)C1]

To a solution of Compound 477 (125 mg, 0.20 mmol) in ethanol (4.00 mL), NaHCO₃ (25.0 mg, 0.30 mmol), H₂O (0.50 mL) and 2-chloroacetaldehyde (0.18 mL, 1.11 mmol) was added. The mixture was stirred at 70° C. for 65 h. The mixture was cooled to room temperature, concentrated in vacuum and diluted with DCM (15 mL), washed with saturated NaHCO₃ aqueous solution (10 mL), saturated brine (10 mL), the organic layers were dried over anhydrous Na₂SO₄, filtered and concentrated under reduce pressure to give the crude which was purified by preparative HPLC (Column: Phenomenex Gemini-NX 80*40 mm*3 um, Mobile Phase A: water (0.05% NH₃H₂O), Mobile Phase B: acetonitrile, Flow rate: 30 mL/min, gradient condition from 38% B to 64%). The pure fractions were collected and the solvent was evaporated under vacuum to give a residue. The residue was partitioned between acetonitrile (4 mL) and water (12 mL). The solution was lyophilized to dryness to give Compound 433 (10.77 mg, 92.64% purity, 5.50% yield) as a gray powder.

### Preparation of Compound 434

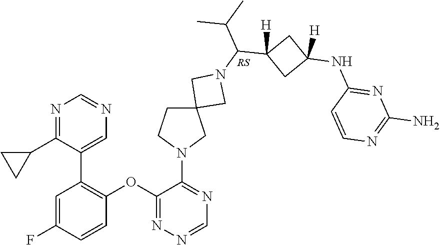 [SMILES: CC(C)C([C@H]1C[C@@H](Nc2ccnc(N)n2)C1)N1CC2(CCN(c3ncnnc3Oc3ccc(F)cc3-c3cncnc3C3CC3)C2)C1]

To a solution of Compound 1 (200.0 mg, 0.37 mmol) in ethanol (6.0 mL) were added 4-chloropyrimidin-2-amine (95.1 mg, 0.73 mmol) and N,N-Diisopropylethylamine (1.21 mL, 7.34 mmol). The resulting mixture was stirred at 80° C. for 22 hours. After cooling to room temperature, the reaction was quenched with water and then extracted with ethyl acetate. The organic layers were combined, washed with brine, dried over sodium sulfate, filtered and concentrated under reduced pressure. The residue was purified by prep-HPLC with the following conditions: Column: Xbridge prep C18 Sum 19*150 mm; Mobile phase A: Waters (10 mmol/L NH₄HCO₃), Mobile phase B: ACN; Flow rate: 25 ml/min; Gradient: 40% B to 50% B in 7 min, 254&220 nm; t: 6.80 min. The fractions containing the desired product were combined and lyophilized to give Compound 434 (4.6 mg, 98.754% purity, 1.94% yield) as a white solid.

### Preparation of Compound 435

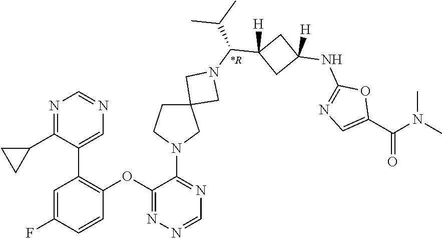 [SMILES: CC(C)[C@H]([C@H]1C[C@@H](Nc2ncc(C(=O)N(C)C)o2)C1)N1CC2(CCN(c3ncnnc3Oc3ccc(F)cc3-c3cncnc3C3CC3)C2)C1]

Compound 479 (180 m g, 0.275 mmol) was dissolved in THE (7.2 mL) and treated with dimethylamine (275 uL, 0.549 mmol, 2.0 equiv, 2 M in THF) and triethylamine (83.3 mg, 0.824 mmol, 2.0 equiv). Then T3P (174.7 mg, 0.549 mmol, 2.0 equiv.) was added. The reaction was stirred at room temperature over night. Further 0.2 equiv. of T3P were added and the mixture was stirred for further 2 hours. The reaction was quenched by addition of water and ethyl acetate. The water layer was separated and extracted with ethyl acetate (1 x). The combined organic phases were washed with sat. Na₂CO₃ and brine, dried over MgSO₄, filtered and concentrated in vacuo. A first purification by column chromatography (silica gel, 0 to 15% MeOH in DCM) was followed by a second purification by Prep HPLC (Stationary phase: RP XBridge Prep C18 OBD-5 μm, 50×250 mm, Mobile phase: 0.25% NH₄HCO₃ solution in water, CH3CN) to give Compound 435 (35 mg, 0.0513 mmol, 19% yield) as a white solid.

The compounds listed in the table below were prepared following the same procedure as reported for the preparation of Compound 435 starting from the corresponding starting materials

| Compound number | Compound structure |
|---|---|
| 436 | 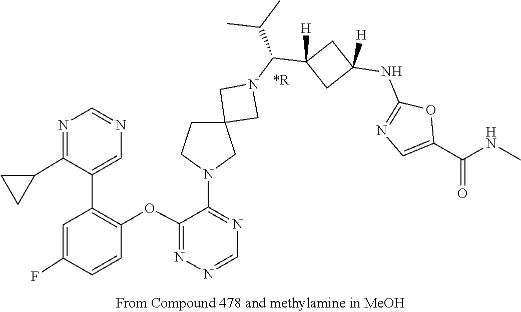 |
| 438 | 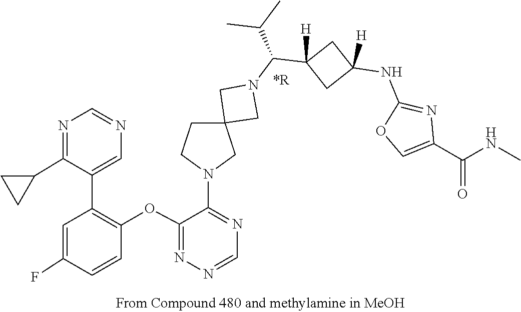 |
| 461 | 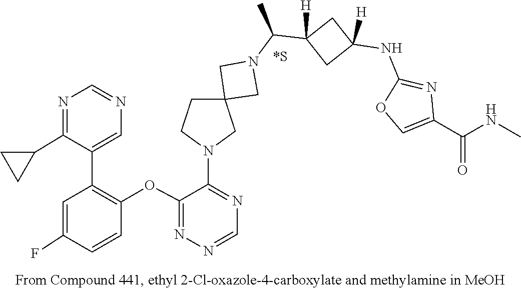 |
| 439 | 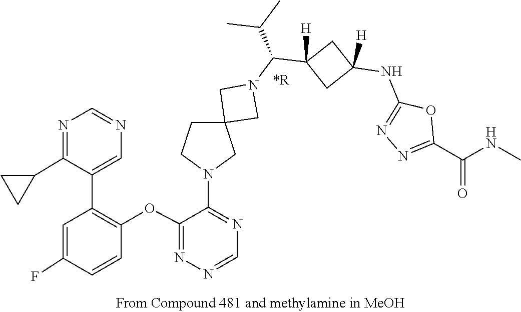 |
| 442 | 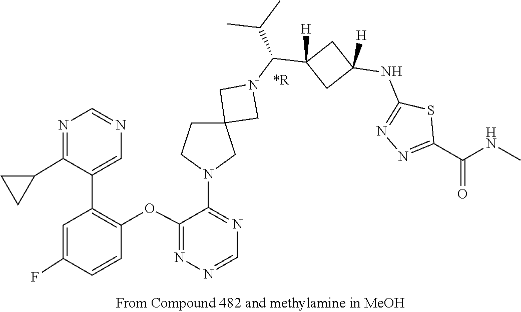 |
| 449 | 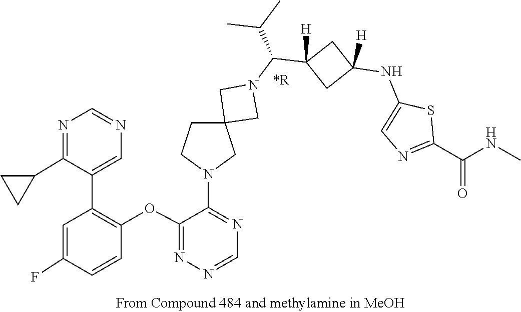 |
| 450 | 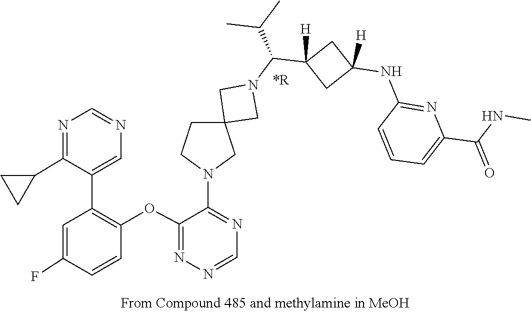 |
| 451 | 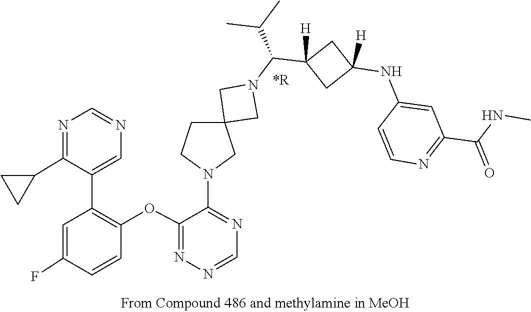 |
| 452 | 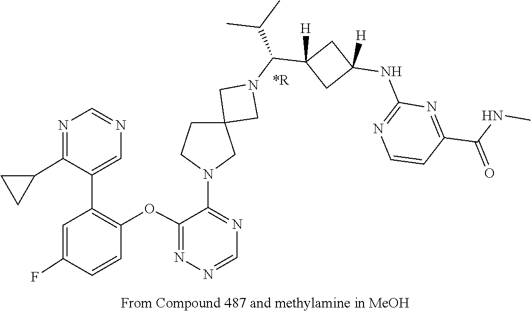 |

### Preparation of Compound 437

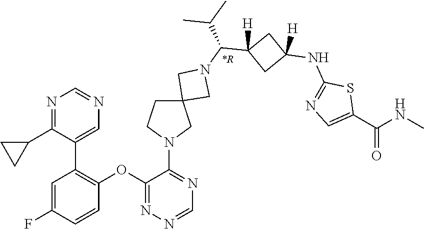 [SMILES: CNC(=O)c1cnc(N[C@H]2C[C@@H]([C@@H](C(C)C)N3CC4(CCN(c5ncnnc5Oc5ccc(F)cc5-c5cncnc5C5CC5)C4)C3)C2)s1]

In a closed vial Compound 1a (305 mg, 0.521 mmol) was treated with 2-bromo-N-methylthiazole-5-carboxamide (120 mg, 0.521 mmol), and JOSIPHOS SL-J009-1 Pd G3 (48.1 mg, 0.0521 mmol). Then dry DMA (5.2 mL) was added. The dark brown mixture was stirred over night at 70° C. Sat sodium carbonate solution and EtOAc was added. The phases were separated and the water phase was extracted several times with EtOAc. The organic phase was dried with magnesium sulfate and filtered. After evaporation of the solvents, the crude product (410 mg) was obtained as a yellow oil. A purification was performed via Prep HPLC (Stationary phase: RP XBridge Prep C18 OBD-10 μm, 30×150 mm, Mobile phase: 0.25% NH₄HCO₃ solution in water, CH₃CN) yielding Compound 437 (121 mg, yield 33.927%) as a white solid.

### Preparation of Compound 512, 512a & 512b

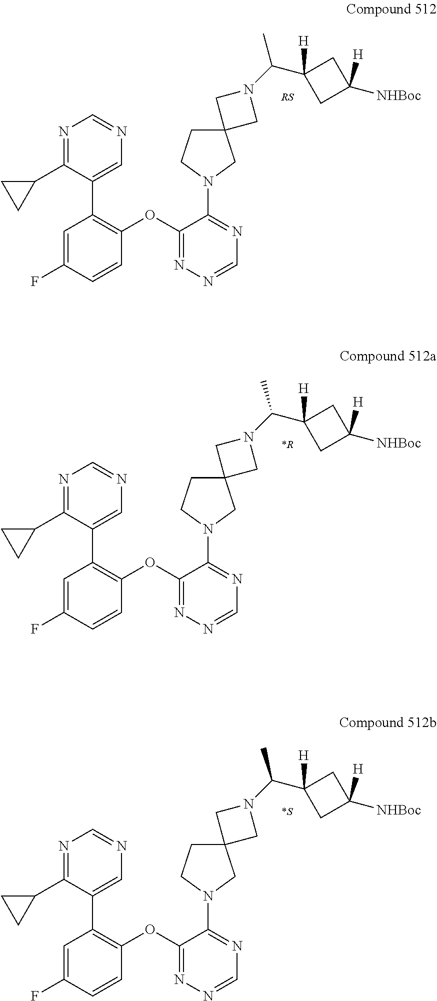

To a solution of intermediate 33 (1.59 g, 3.61 mmol), acetic acid (216.8 mg, 3.61 mmol) and intermediate 336 (1.54 g, 7.221 mmol) were dissolved in dry methanol (20 mL). Then sodium cyanoborohydride (907.5 mg, 14.4 mmol) was added. After stirring at 60° C. over night, the methanol was evaporated. Ethyl acetate was added. Then a saturated solution of sodium carbonate was added and the water phase was further basified with 1 N NaOH solution to pH 13. The water phase was extracted with EtOAc and DCM several times. The combined organic layers were washed with brine and dried over anhydrous magnesium sulfate. The solid was filtered off. The filtrate was concentrated under reduced pressure. A crude product (2 g) was obtained as light yellow solid and purified by prep CC (silica gel, 2% to 10% MeOH in DCM) to give two fractions of Compound 512 (700 mg, 31.4% yield) and (1000 mg, 44.9% yield) with various amounts of impurities as a white solids.

A purification was performed via Prep SFC (Stationary phase: Chiralpak Daicel IG 20×250 mm, Mobile phase: CO₂, EtOH+0.4 iPrNH₂) to give Compound 512a (680 mg, 30.5% yield) and Compound 512b (630 mg, 28.3% yield) were obtained as a colorless oils.

### Preparation of Compound 440

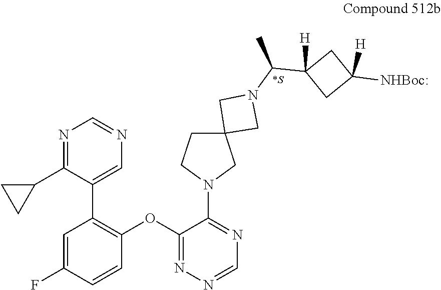

In a flask, Compound 512a (660 mg, 1.07 mmol) was dissolved in DCM (10.3 mL) and treated with TFA (1.31 mL, 17.122 mmol) at 0° C. After stirring over night, thereby the mixture was allowed to come to rt. The mixture was diluted with DCM and sat. sodium carbonate solution. The water was adjusted to pH 13 with sodium hydroxide solution. The water phase was extracted multiple times with DCM and EtOAc. The combined organic solvents were dried with MgSO₄, filtrated and evaporated to obtain Compound 440 (550 mg, 99.5% yield) as a white foamy solid. A part of the product (35 mg) was used and a purification was performed via Prep HPLC (Stationary phase: RP XBridge Prep C18 OBD-10 μm, 30×150 mm, Mobile phase: 0.25% NH₄HCO₃ solution in water, CH₃CN) yielding Compound 440 (30 mg) as a white solid.

### Preparation of Compound 441

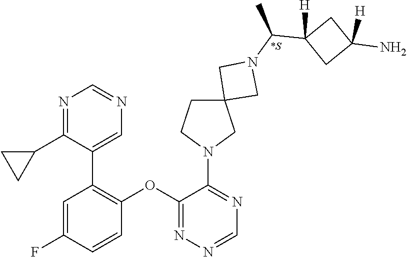 [SMILES: C[C@@H]([C@H]1C[C@@H](N)C1)N1CC2(CCN(c3ncnnc3Oc3ccc(F)cc3-c3cncnc3C3CC3)C2)C1]

In a flask, Compound 512b (600 mg, 0.973 mmol) was dissolved in DCM (9.4 mL) and treated with TFA (1.19 mL, 15.6 mmol) at 0° C. After stirring over night, thereby the mixture was allowed to come to rt. The mixture was diluted with DCM and sat. sodium carbonate solution. The water was adjusted to pH 13 with sodium hydroxide solution. The water phase was extracted multiple times with DCM and EA. The combined organic solvents were dried with MgSO₄, filtrated and evaporated to obtain Compound 441 (530 mg, quant. yield) as a white foamy solid. A part of the product (33 mg) was used and a purification was performed via Prep HPLC (Stationary phase: RP XBridge Prep C18 OBD-10 μm, 30×150 mm, Mobile phase: 0.25% NH₄HCO₃ solution in water, CH₃CN) yielding Compound 441 (19.8 mg) as a white solid.

### Preparation of Compound 443

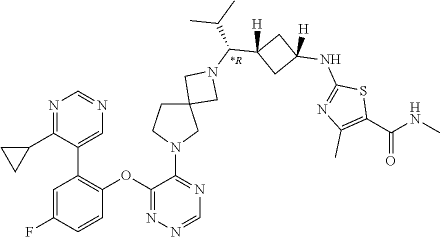 [SMILES: CNC(=O)c1sc(N[C@H]2C[C@@H]([C@@H](C(C)C)N3CC4(CCN(c5ncnnc5Oc5ccc(F)cc5-c5cncnc5C5CC5)C4)C3)C2)nc1C]

Bis(trimethylaluminium)-1,4-diazabicyclo[2.2.2]octane adduct (0.18 g, 0.71 mmol) was added portionwise to a stirred solution of Compound 482 (130 mg, 0.18 mmol) and methylamine, 2 M in THE (0.54 mL, 1.07 mmol) in anhydrous toluene (10 mL). After addition the reaction mixture was stirred at 100° C. for 5 hours. The solvents were evaporated using a flow of nitrogen gas while heating at 65° C. The residue was dissolved in dichloromethane and some methanol. The resulting suspension was filtered over a pad of Dicalite. The pad was washed with dichloromethane with some methanol. The solvents of the filtrate were evaporated under reduced pressure at 45° C. The residue was dissolved in dichloromethane and purified over a SiO₂ column, 12 g, using dichloromethane and methanol as eluents in a gradient starting from 100% dichloromethane and ending with 90% dichloromethane and 10% methanol. The fractions containing product were combined and the solvents were evaporated under reduced pressure at 50° C. to give Compound 443 (80 mg, 64% yield).

The compounds listed in the table below were prepared following the same procedure as reported for the preparation of Compound 443 starting from the corresponding starting materials

| Compound number | Compound structure |
|---|---|
| 459 | 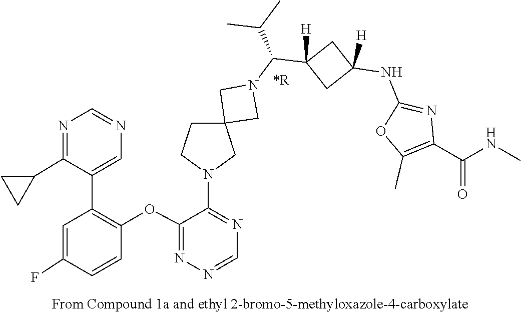 |
| 460 | 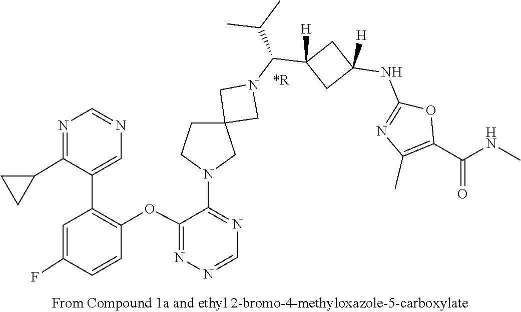 |

### Preparation of Compound 444

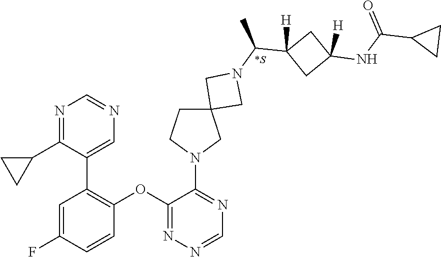 [SMILES: C[C@@H]([C@H]1C[C@@H](NC(=O)C2CC2)C1)N1CC2(CCN(c3ncnnc3Oc3ccc(F)cc3-c3cncnc3C3CC3)C2)C1]

In a small vial, Compound 441 (22 mg, 0.0426 mmol) was dissolved in dry DCM (0.46 mL). Then cyclopropanecarboxylic acid (4.4 mg, 0.0511 mmol), Et₃N (13.0 mg, 0.728 g/mL, 0.128 mmol), and T3P, 50% in ethyl acetate (38.0 mg, 0.0597 mmol) was added. The mixture was stirred over night at rt. The solvents were evaporated and the crude product which was directly subjected to Prep HPLC (Stationary phase: RP XBridge Prep C18 OBD-10 μm, 30×150 mm, Mobile phase: 0.25% NH₄HCO₃ solution in water, CH3CN) yielding Compound 444 (12 mg, 48.2% yield) as a white solid.

### Preparation of Compound 445

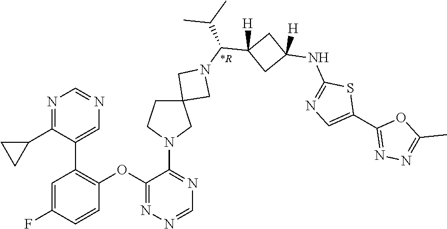 [SMILES: Cc1nnc(-c2cnc(N[C@H]3C[C@@H]([C@@H](C(C)C)N4CC5(CCN(c6ncnnc6Oc6ccc(F)cc6-c6cncnc6C6CC6)C5)C4)C3)s2)o1]

In a flask, Compound 1a (50.0 mg, 0.0918 mmol) was treated with K₂CO₃ (25.4 mg, 0.184 mmol) and intermediate 338 (55.5 mg, 0.138 mmol). Then dry acetonitrile (1.4 mL) was added and the mixture was heated to 75° C. (red mixture obtained). After stirring over night, a 1:1 mixture of Compound 1a and product was observed. 20 mg more of the base and 20 mg more of intermediate 338 were added. The mixture was stirred for 8 h at 75° C. Nearly no sm was observed anymore in HPLC. After cooling down to RT, the mixture was allowed to stand over 3 days at RT. Water and ethyl acetate were added and the separated water phase was extracted several times with ethyl acetate. The collected organic phases were dried MgSO₄, filtered and evaporated at rotavap to give the crude product which was purified by Prep HPLC (Stationary phase: RP XBridge Prep C18 OBD-5 μm, 50×250 mm, Mobile phase: 0.25% NH₄HCO₃ solution in water, CH₃CN) to afford Compound 445 (15.3 mg, 23.5% yield) as a white solid.

### Preparation of Compound 446

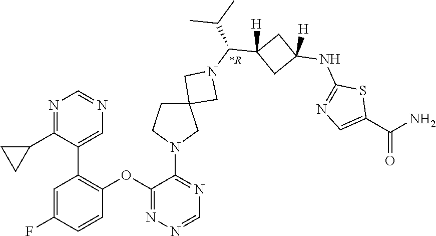 [SMILES: CC(C)[C@H]([C@H]1C[C@@H](Nc2ncc(C(N)=O)s2)C1)N1CC2(CCN(c3ncnnc3Oc3ccc(F)cc3-c3cncnc3C3CC3)C2)C1]

In a small vial, Compound 1a (50 mg, 0.0918 mmol) was dissolved in dry MeCN (1.4 mL) and treated with K₂CO₃ (25.4 mg, 0.184 mmol) and 2-bromo thiazole 5-carboxamide (22.8 mg, 0.11 mmol). The mixture was heated in a closed vial at 70° C. for 7 days. The mixture was deep red. HPLC showed full conversion. Sodium carbonate solution (sat.) was added and the water phase was extracted several times with EtOAc. Drying with magnesium sulfate, filtration on evaporation afforded a crude product which was purified by Prep HPLC (Stationary phase: RP XBridge Prep C18 OBD-10 μm, 30×150 mm, Mobile phase: 0.25% NH₄HCO₃ solution in water, CH₃CN) yielding Compound 446 (15.3 mg, 24.8% yield) as a white fluffy solid.

### Preparation of Compound 447

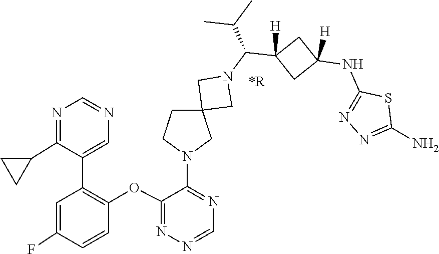 [SMILES: CC(C)[C@H]([C@H]1C[C@@H](Nc2nnc(N)s2)C1)N1CC2(CCN(c3ncnnc3Oc3ccc(F)cc3-c3cncnc3C3CC3)C2)C1]

Compound 1a (40 mg, 0.0734 mmol), 5-bromo-1,3,4-thiadiazol-2-amine (13.221 mg, 0.0734 mmol) and DIPEA (0.0633 mL, 0.75 g/mL, 0.367 mmol) were added to MeCN (4 mL, 0.786 g/mL, 76.585 mmol). The mixture was stirred at 75° C. for 2 hours. The solvent was removed and the residue was purified by flash column (C18, CH₃CN:H₂O from 0:100 to 50:50, 0.5% fumarate as buffer) to afford Compound 447 (25 mg, 48% yield).

### Preparation of Compound 448

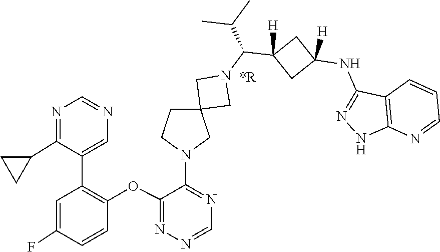 [SMILES: CC(C)[C@H]([C@H]1C[C@@H](Nc2n[nH]c3ncccc23)C1)N1CC2(CCN(c3ncnnc3Oc3ccc(F)cc3-c3cncnc3C3CC3)C2)C1]

A mixture of Compound 1a (200 mg, 0.367 mmol), 3-iodo-1-(tetrahydro-2H-pyran-2-yl)-1H-pyrazolo[3,4-b]pyridine (241.715 mg, 0.734 mmol), L-proline (42.275 mg, 0.367 mmol), CuI (34.966 mg, 0.184 mmol) and K₂CO₃ (152.244 mg, 1.102 mmol) in DMSO (23.676 mL, 1.092 g/mL, 330.894 mmol) was stirred at 115° C. overnight. The reaction mixture was diluted with EtOAc, washed with water and brine, dried, filtered and concentrated to give the crude product. Purification by Biotage (C18, 5-95% MeCN in water with 0.05% formic acid) to afford the THP protected intermediate (130 mg, yield 47.464%), which was treated with TFA and DCM and stirred for 2 h, concentrated and purified by Biotage (C18, 5-95% MeCN in water with 0.05% formic acid) to afford Compound 448 (80 mg, 31% yield) as a white solid.

The compounds listed in the table below were prepared following the same procedure as reported for the preparation of Compound 448 starting from the corresponding starting materials

| Compound number | Compound structure |
|---|---|
| 458 | 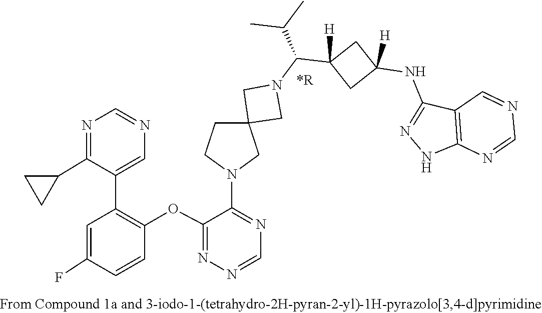 |

### Preparation of Compound 453

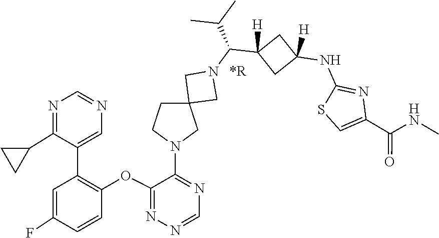 [SMILES: CNC(=O)c1csc(N[C@H]2C[C@@H]([C@@H](C(C)C)N3CC4(CCN(c5ncnnc5Oc5ccc(F)cc5-c5cncnc5C5CC5)C4)C3)C2)n1]

In a closed vial, Compound 1a (150 mg, 0.256 mmol) was treated with 2-bromo-N-methylthiazole-4-carboxamide (56.6 mg, 0.256 mmol), JOSIPHOS SL-J009-1 Pd G3 (23.7 mg, 0.0256 mmol), and Cs₂CO₃ (250.3 mg, 0.768 mmol). Then dry DMA (2.6 mL) was added. The dark brown mixture was stirred over two days at 70° C. Sat. sodium carbonate solution was added along with EtOAc. The water phase was extracted several times with EtOAc. The organic phase were dried with magnesium sulfate. After filtration and evaporation, the crude product was purified via Prep HPLC (Stationary phase: RP XBridge Prep C18 OBD-10 μm, 30×150 mm, Mobile phase: 0.25% NH₄HCO₃ solution in water, CH₃CN) to obtain Compound 453 (35.6 mg, 20.3% yield) as a white solid.

### Preparation of Compound 454

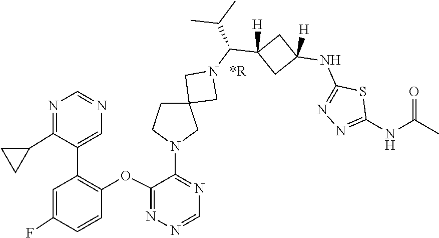 [SMILES: CC(=O)Nc1nnc(N[C@H]2C[C@@H]([C@@H](C(C)C)N3CC4(CCN(c5ncnnc5Oc5ccc(F)cc5-c5cncnc5C5CC5)C4)C3)C2)s1]

Compound 488 (130 mg, 0.192 mmol) was dissolved in DCM (4.75 mL, 1.326 g/mL, 74.159 mmol). DIEA (0.0496 mL, 0.75 g/mL, 0.288 mmol) and acetic anhydride (23.501 mg, 0.23 mmol) was added to the mixture at 0° C. dropwise. The solvent was removed and the residue was purified by flash column (C18, CH₃CN: H₂O from 5:95 to 30:70, HCOOH as buffer) to afford Compound 454 (88 mg, 65% yield).

### Preparation of Compound 455

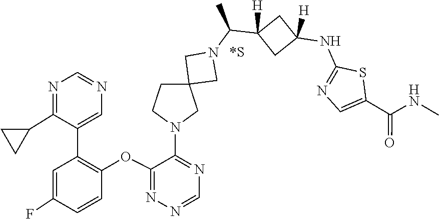 [SMILES: CNC(=O)c1cnc(N[C@H]2C[C@@H]([C@H](C)N3CC4(CCN(c5ncnnc5Oc5ccc(F)cc5-c5cncnc5C5CC5)C4)C3)C2)s1]

In a closed vial, Compound 441 (94 mg, 0.182 mmol) was treated with 2-bromo-N-methylthiazole-5-carboxamide (49.7 mg, 0.2 mmol), JOSIPHOS SL-J009-1 Pd G3 (16.8 mg, 0.0182 mmol), and Cs₂CO₃ (177.9 mg, 0.546 mmol). Then dry DMA (1.8 mL) was added. The dark brown mixture was stirred over night at 7° C. Sat sodium carbonate solution and EtOAc was added. The phases were separated and the water phase was extracted several times with EtOAc. The organic phase was dried with magnesium sulfate and filtered. After evaporation of the solvents, the crude product was obtained as a yellow solid. A purification was performed FCC (silica gel, 5% to 10% methanol in DCM) to afford the product (33 mg, 93% purity) as white solid, which was further purified by Prep HPLC (Stationary phase: RP XBridge Prep C18 OBD-5 μm, 50×250 mm, Mobile phase: 0.25% NH₄HCO₃ solution in water, CH₃CN) to give Compound 455 (19 mg, 16% yield).

### Preparation of Compound 456

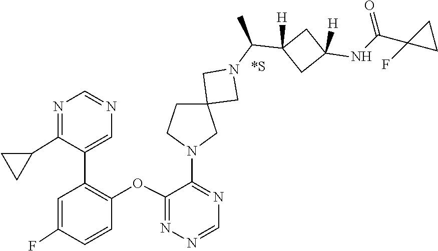 [SMILES: C[C@@H]([C@H]1C[C@@H](NC(=O)C2(F)CC2)C1)N1CC2(CCN(c3ncnnc3Oc3ccc(F)cc3-c3cncnc3C3CC3)C2)C1]

In a small vial, Compound 441 (30 mg, 0.0581 mmol) was dissolved in dry DCM (0.63 mL). Then cyclopropane fluoro carboxylic acid (7.3 mg, 0.0697 mmol), Et₃N (24 μL, 0.728 g/mL, 0.174 mmol), and T3P, 50% in ethyl acetate (51.8 mg, 0.0814 mmol) was added. The mixture was stirred over night at rt. The solvents were evaporated and the solid was directly subjected to Prep HPLC (Stationary phase: RP XBridge Prep C18 OBD-10 μm, 30×150 mm, Mobile phase: 0.25% NH₄HCO₃ solution in water, CH₃CN) yielding Compound 456 (3.6 mg, 10.3% yield) as a white solid.

### Preparation of Compound 457

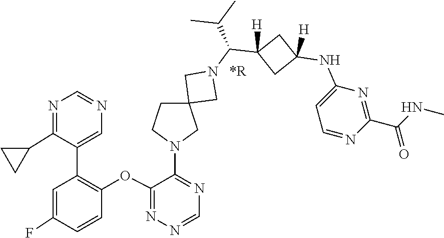 [SMILES: CNC(=O)c1nccc(N[C@H]2C[C@@H]([C@@H](C(C)C)N3CC4(CCN(c5ncnnc5Oc5ccc(F)cc5-c5cncnc5C5CC5)C4)C3)C2)n1]

In a vial, Compound 489 (141 mg, 0.207 mmol) was dissolved in MeNH₂ (2 M in isopropanol) (4.97 mL, 2 M, 9.941 mmol) and heated at 70° C. over night. The solvents were removed at rotavap. The residue was diluted in 18 mL MeCN and subjected to prep HPLC (Stationary phase: RP XBridge Prep C18 OBD-10 μm, 30×150 mm, Mobile phase: 0.25% NH₄HCO₃ solution in water, CH₃CN) yielding Compound 457 (73 mg, 51.8% yield) as a white fluffy solid after lyophilzation.

### Preparation of Compound 462

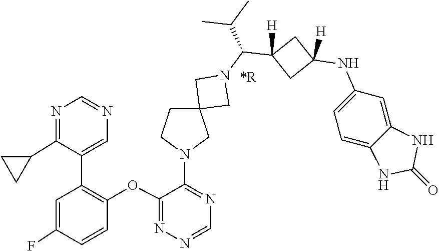 [SMILES: CC(C)[C@H]([C@H]1C[C@@H](Nc2ccc3[nH]c(=O)[nH]c3c2)C1)N1CC2(CCN(c3ncnnc3Oc3ccc(F)cc3-c3cncnc3C3CC3)C2)C1]

A mixture of Compound 38b (100 mg, 0.184 mmol), 5-amino-1,3-dihydro-2h-benzimidazol-2-one (54.871 mg, 0.368 mmol), AcOH (0.0211 mL, 1.049 g/mL, 0.368 mmol) in MeOH (3 mL, 0.791 g/mL, 74.059 mmol) was stirred at 50° C. for 30 min before the addition of sodium cyanoborohydride (23.119 mg, 0.368 mmol). The mixture was stirred at 50° C. for 3 hr and concentrated. The crude product was purified by ISCO (C18, 5-95% MeCN in water with 0.05% formic acid) to afford Compound 462 (25 mg, 18% yield).

### Preparation of Compound 463

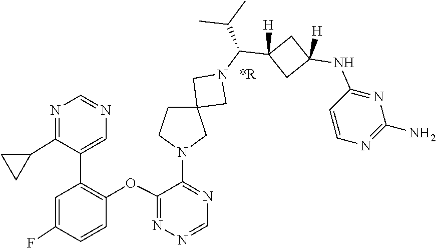 [SMILES: CC(C)[C@H]([C@H]1C[C@@H](Nc2ccnc(N)n2)C1)N1CC2(CCN(c3ncnnc3Oc3ccc(F)cc3-c3cncnc3C3CC3)C2)C1]

4-chloropyrimidin-2-amine (105 mg, 0.808 mmol) was added to a solution of Compound 1a (200 mg, 0.367 mmol), TEA (74.3 mg, 0.734 mmol) in propan-2-ol (5 mL). The mixture was stirred at 80° C. for overnight. The reaction mixture was concentrated under reduced pressure to afford the crude product which was purified by SFC over DAICEL CHIRALPAK AD (250 mm*30 mm, 10 um) (eluent: 40% to 40% (v/v) supercritical 0.1% NH₃H₂O EtOH). The pure fractions were collected and the volatiles were removed under reduced pressure. The product was suspended in water (5 mL), the mixture frozen using dry ice/acetone, and then lyophilized to dryness to afford Compound 463 (68 mg, 29. % yield) as a white solid.

### Preparation of Compound 510

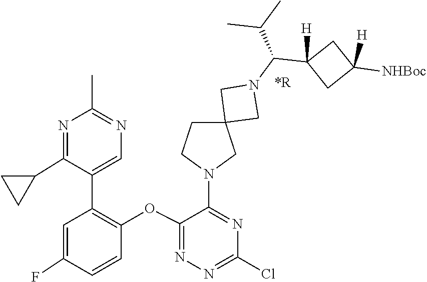 [SMILES: Cc1ncc(-c2cc(F)ccc2Oc2nnc(Cl)nc2N2CCC3(C2)CN([C@H](C(C)C)[C@H]2C[C@@H](NC(=O)OC(C)(C)C)C2)C3)c(C2CC2)n1]

To a solution of intermediate 321 (0.850 g, 3.48 mmol) and intermediate 333 (1.69 g, 3.48 mmol) in dry tetrahydrofuran (30 mL) was added DBU (0.636 g, 4.18 mmol). The reaction mixture was stirred at 25° C. for 12 hours. The reaction mixture was diluted with dichloromethane (50 mL) and water (50 mL) was added. The mixture was extracted with dichloromethane (50 mL×3). The combined organic layers were dried over anhydrous Na₂SO₄, filtered and concentrated under reduced pressure to give the crude product, which was purified by flash column chromatography on silica gel (eluent: DCM:MeOH from 1:0 to 10:1) to give Compound 510 (2.50 g, crude) as a yellow solid.

### Preparation of Compound 511

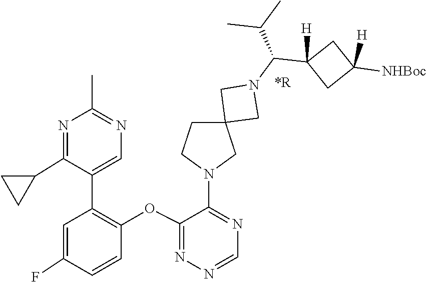 [SMILES: Cc1ncc(-c2cc(F)ccc2Oc2nncnc2N2CCC3(C2)CN([C@H](C(C)C)[C@H]2C[C@@H](NC(=O)OC(C)(C)C)C2)C3)c(C2CC2)n1]

To a solution of Compound 510 (2.50 g, crude) in THE (50 mL) was added TMEDA (0.838 g, 7.21 mmol) and Pd(dppf)₂Cl₂ (0.132 g, 0.180 mmol) and NaBH₄ (0.408 g, 6.49 mmol). The mixture was stirred at 25° C. for 8 h under N₂. The reaction was quenched with MeOH (50 mL) dropwise, and the mixture was diluted with water (50 mL) and extracted with EtOAc (50 mL×4). The organic layers were dried over Na₂SO₄, filtered and concentrated, and the residue was purified by FCC (from pure DCM to pure DCM/MeOH=10/1) to give Compound 511 (1.30 g, yield: 49.2%) as a yellow solid.

### Preparation of Compound 464

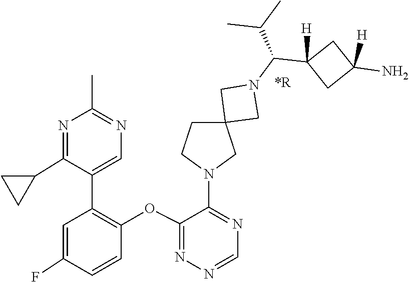 [SMILES: Cc1ncc(-c2cc(F)ccc2Oc2nncnc2N2CCC3(C2)CN([C@H](C(C)C)[C@H]2C[C@@H](N)C2)C3)c(C2CC2)n1]

To a solution of Compound 511 (1.30 g, 1.85 mmol) in DCM (6 mL) was added TFA (2 mL, 26.9 mmol). The mixture was stirred at 25° C. for 1 h. The mixture was concentrated, the residue was diluted with DCM (20 mL) and neutralized with cold 2 M NaOH (14 mL). The mixture was extracted with DCM (25 mL×5) and the organic layers were dried over Na₂SO₄, filtered and concentrated to give Compound 464 (1.1 g, crude) as a yellow solid.

### Preparation of Compound 465

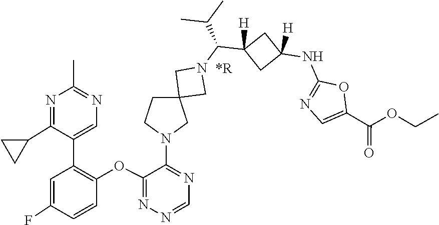 [SMILES: CCOC(=O)c1cnc(N[C@H]2C[C@@H]([C@@H](C(C)C)N3CC4(CCN(c5ncnnc5Oc5ccc(F)cc5-c5cnc(C)nc5C5CC5)C4)C3)C2)o1]

A stir bar, Compound 464 (50 mg, 0.089 mmol), ethyl 2-chlorooxazole-5-carboxylate (17.3 mg, 0.099 mmol), K₂CO₃ (24.7 mg, 0.179 mmol) in ACN (3 mL) were added to a 8 mL round-bottomed flask before the mixture was stirred at rt for 8 h. The solvent was evaporated under reduced pressure to give crude Compound 465 (70 mg, crude) as a brown solid.

The compounds listed in the table below were prepared following the same procedure as reported for the preparation of Compound 465 starting from the corresponding starting materials

| Compound number | Compound structure |
|---|---|
| 467 | 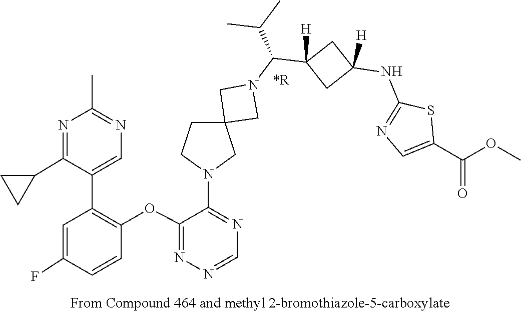 |
| 473 | 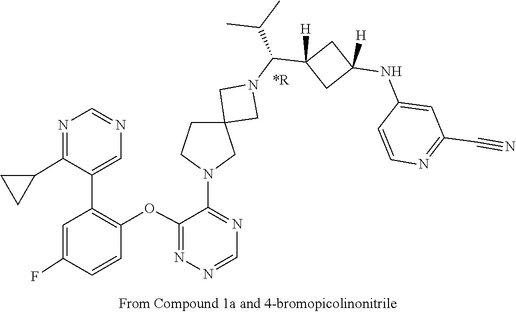 |
| 478 | 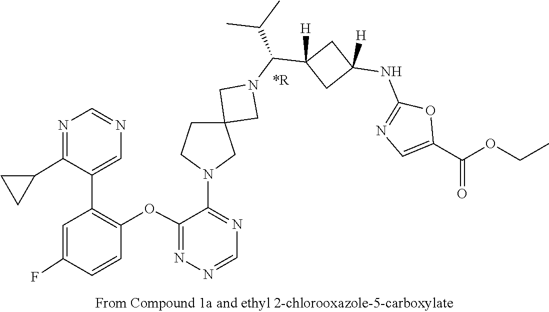 |

### Preparation of Compound 466

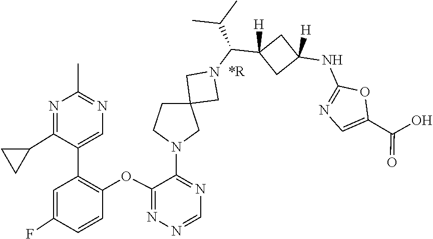 [SMILES: Cc1ncc(-c2cc(F)ccc2Oc2nncnc2N2CCC3(C2)CN([C@H](C(C)C)[C@H]2C[C@@H](Nc4ncc(C(=O)O)o4)C2)C3)c(C2CC2)n1]

To a solution of Compound 465 (120 mg, 0.17 mmol) in THF/H₂O (4 mL/1.5 mL) was added LiOH·H₂O (7 mg, 0.17 mmol) and the mixture was stirred at 25° C. for 10 h. The residue was basified with HCl (1N aqueous) to pH=4 and concentrated under reduced pressure to give the crude product as a white solid (120 mg, crude).

The compounds listed in the table below were prepared following the same procedure as reported for the preparation of Compound 466 starting from the corresponding starting materials

| Compound number | Compound structure |
|---|---|
| 468 | 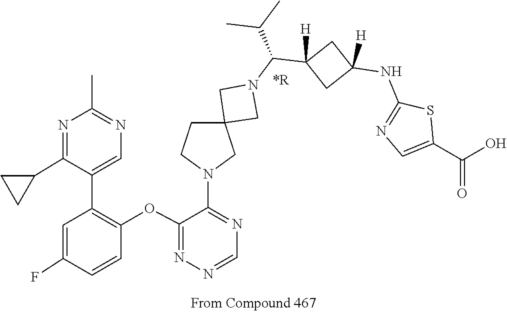 |
| 479 | 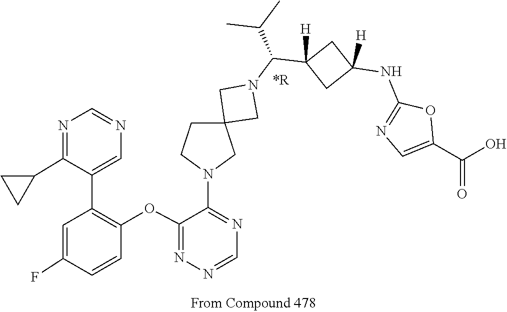 |

### Preparation of Compound 470

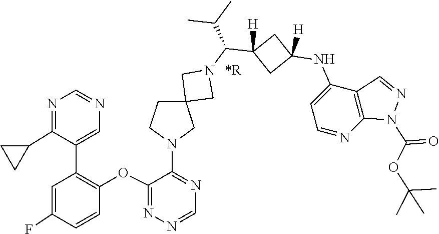 [SMILES: CC(C)[C@H]([C@H]1C[C@@H](Nc2ccnc3c2cnn3C(=O)OC(C)(C)C)C1)N1CC2(CCN(c3ncnnc3Oc3ccc(F)cc3-c3cncnc3C3CC3)C2)C1]

A stir bar, t-BuXPhos-Pd-G3 (14.6 mg, 0.018 mmol), t-BuXPhos (8.0 mg, 0.019 mmol), Compound 1a (100.0 mg, 0.184 mmol) and tert-butyl 4-bromo-1H-pyrazolo[3,4-b]pyridine-1-carboxylate (82.1 mg, 0.275 mmol) were added to a 50 mL round-bottomed flask. The resulting mixture was purged with argon for three times, then LiHMDS (0.92 mL, 0.92 mmol, 1 M in THF) and anhydrous tetrahydrofuran (8 mL) were added. The reaction mixture was purged with argon for three times again and heated to 65° C. and stirred for 12 hours. The reaction mixture was cooled to room temperature and quenched with HCl (1 M, 3 mL), diluted with ethyl acetate (50 mL) and poured into saturated solution of sodium bicarbonate (50 mL). The mixture was extracted with ethyl acetate (30 mL×3). The combined organic layers were dried over anhydrous Na₂SO₄, filtered and concentrated under reduced pressure to give a residue, which was purified by flash column chromatography on silica gel (eluent: dichloromethane:methanol from 1:0 to 10:1, TLC: dichloromethane:methanol=10:1, Rf=0.5) to give Compound 470 (45.0 mg, 69.71% purity, 22.4% yield) as a brown solid.

### Preparation of Compound 471

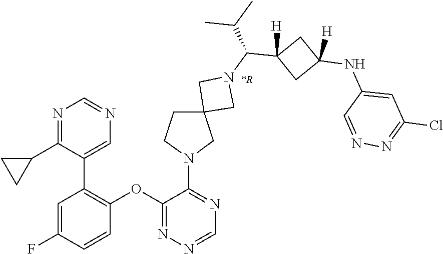

Compound 1a (200 mg, 0.367 mmol), 3,5-dichloropyridazine (82 mg, 0.55 mmol), Et₃N (0.16 ml, 1.2 mmol), and DMSO (2 mL) were added to a 10 mL sealed tube. The resultant mixture was stirred at 100° C. for 12 hours before cooling to room-temperature. The residue was purified by preparative HPLC using a Phenomenex Gemini-NX 150*30 mm*5 um (eluent: 41% to 69% (v/v) CH₃CN and water (0.05% NH₃H₂O)-ACN) to afford pure product. The product was suspended in water (4 mL), the mixture frozen using dry ice/EtOH, and then lyophilized to dryness to afford Compound 471 (75 mg, 30.9%) as a yellow solid.

The compounds listed in the table below were prepared following the same procedure as reported for the preparation of Compound 471 starting from the corresponding starting materials

| Compound number | Compound structure |
|---|---|
| 475 | 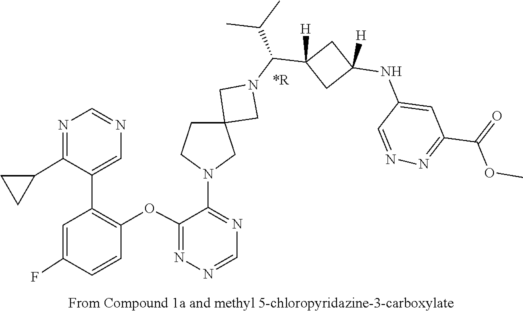 |

### Preparation of Compound 472

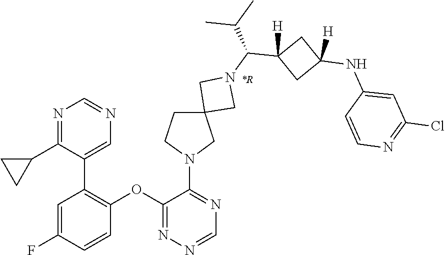

To a solution of Compound 1a (300 mg, 0.551 mmol) in NMP (3 ml) was added 2-chloro-4-fluoropyridine (150.0 mg, 1.14 mmol) and DIEA (216 mg, 1.671 mmol) in a microwave apparatus. The mixture was stirred at 150° C. for 0.5 hours. The mixture was purified by reverse phase silica gel column (Column: 120 g Agela C18 150*25 mm*5 um, Mobile Phase A: water, Mobile Phase B: acetonitrile, Flow rate: 30 mL/min, gradient condition: from 80% B to 100% B) to afford Compound 472 (350 mg, 90% yield) as a brown solid.

### Preparation of Compound 474

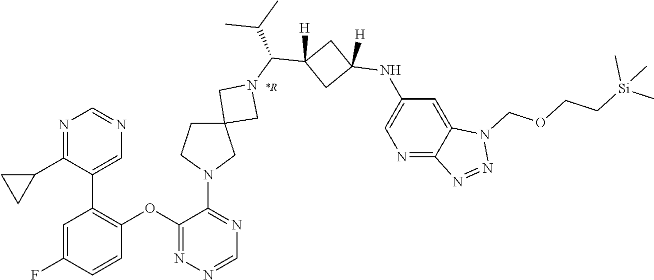

To a suspension of Compound 1a (150 mg, 0.275 mmol) in 1,4-dioxane (3 ml) was added 6-bromo-1-((2-(trimethylsilyl)ethoxy)methyl)-1H-[1,2,3]triazolo[4,5-b]pyridine (305 mg, 0.824 mmol), t-BuONa (90 mg, 0.936 mmol) and tBuXPhos-Pd-G3 (15 mg, 0.019 mmol) with Ar₂ in a microwave apparatus. The mixture was stirred at 100° C. for 2 hours. The mixture was filtered and the filtrate was concentrated under reduced pressure. The crude product was purified by reverse phase silica gel column (Column: 20 g Agela C18 150*25 mm*5 um, Mobile Phase A: water, Mobile Phase B: acetonitrile, Flow rate: 35 mL/min, gradient condition: from 35% B to 60% B) to afford Compound 474 (174 mg, 80% yield) as a yellow solid.

### Preparation of Compound 476

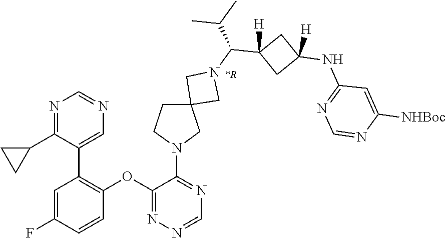

To a mixture of Compound 1a (500 mg, 0.918 mmol), tert-butyl (6-chloropyrimidin-4-yl)carbamate (421 mg, 1.83 mmol) in EtOH (8 mL), then DIEA (0.48 mL, 2.76 mmol) was added to the above mixture. The mixture was stirred at 80° C. for 48 hours. The mixture was concentrated in vacuo. The residue was diluted with DCM (20 ml), washed with water (10 mL), dried over anhydrous Na₂SO₄, filtered and concentrated under reduce pressure to give the crude which was purified by FCC (eluting with dichloromethane:MeOH=1:0 to 10:1) to give Compound 476 (226 mg, purity 86.8%, yield 29%) as a yellow solid.

### Preparation of Compound 477

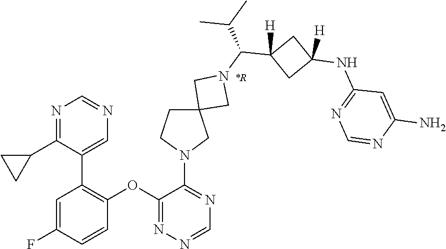

To a mixture of Compound 476 (220 mg, 0.298 mmol) in DCM (3 mL) was added TFA (1 mL) at 0° C. The mixture was stirred for 1 hour at room temperature. The mixture evaporated to remove solvent. The residue was diluted by water (5 mL), basified to pH=12 with NaOH (2 M, 4 mL), extracted with DCM (10 mL×3). The combined organic dried over Na₂SO₄, filtered, and concentrated to dryness under reduce pressure to afford Compound 477 (173 mg, 91% yield) as a yellow solid.

### Preparation of Compound 480

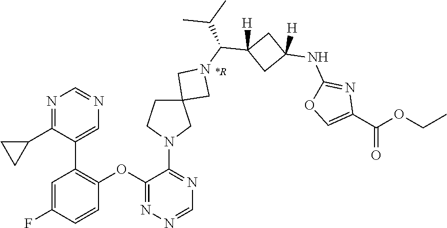

In a vial, ethyl 2-chlorooxazole-4-carboxylate (0.099 g, 0.55 mmol) was added to a stirred mixture of Compound 1a (250 mg, 0.46 mmol) and K₂CO₃ (0.13 g, 0.92 mmol) in DMF (6 mL, 0.94 g/mL, 77.16 mmol) at room temperature. After addition the reaction mixture was stirred at 90° C. for 4 hours. The reaction mixture was diluted with ethylacetate and then filtered. The filtrate was washed with brine, dried with MgSO₄, filtered and the solvents of the filtrate evaporated under reduced pressure at 55° C. The residue was dissolved in dichloromethane and purified over a SiO₂ column, 12 g, using dichloromethane and methanol as eluents in a gradient starting from 100% dichloromethane and ending with 95% dichloromethane and 5% methanol. The fractions containing product were combined and the solvents were evaporated under reduced pressure at 50° C. to give Compound 480 (170 mg, 54% yield).

The compounds listed in the table below were prepared following the same procedure as reported for the preparation of Compound 480 starting from the corresponding starting materials

| Compound number | Compound structure |
|---|---|
| 481 | 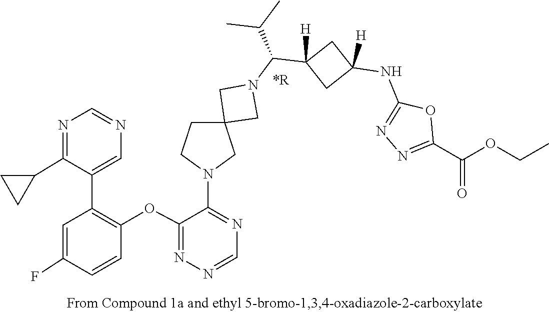 |
| 482 | 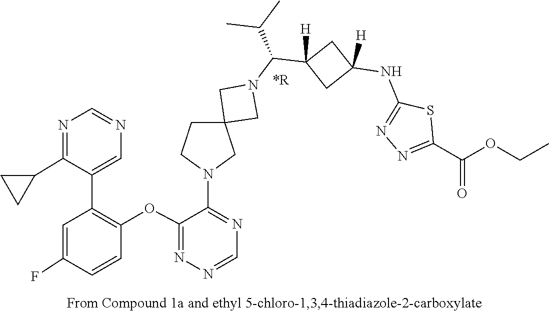 |
| 483 | 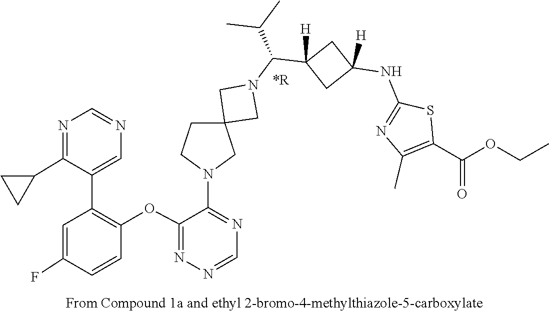 |
| 487 | 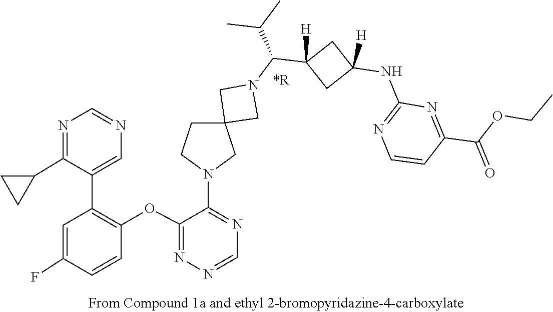 |

### Preparation of Compound 484

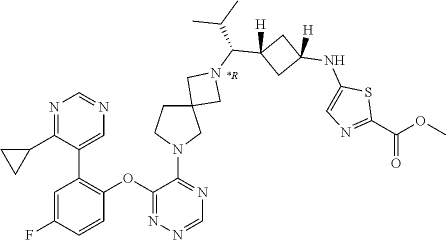

Compound 1a (100 mg, 0.184 mmol), methyl 5-bromothiazole-2-carboxylate (40.77 mg, 0.184 mmol), BrettPhos Pd G3 (16.643 mg, 0.0184 mmol) and Cs₂CO₃ (179.457 mg, 0.551 mmol) were added to toluene (7.5 mL, 0.867 g/mL, 70.572 mmol). The mixture was heated to 110° C. for 16 hours under N₂ protection. Solvent was removed and the residue was purified by flash column (PE:EtOAc from 70:30 to 0:100) to afford Compound 484 (20 mg, 36% yield).

### Preparation of Compound 485

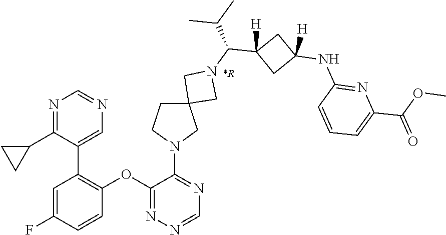

A mixture of Compound 1a (250 mg, 0.43 mmol), methyl 6-bromopicolinate (0.18 g, 0.85 mmol) and cesium carbonate (0.42 g, 1.28 mmol) in 1,4-dioxane (3 mL) was flushed through with nitrogen gas. Then, palladium(II) acetate (0.0096 g, 0.043 mmol) and 4,5-bis(diphenylphosphino)-9,9-dimethylxanthene (0.025 g, 0.043 mmol) were added. The reaction mixture was purged with N₂ and heated at 100° C. for 18 h. The reaction mixture was cooled down to room temperature, diluted with ethylacetate and then filtered. The filtrate was washed with water and brine, dried with MgSO₄, filtered and the solvents of the filtrate evaporated under reduced pressure at 45° C. The residue was dissolved in dichloromethane and purified over a SiO₂ column, 12 g, using dichloromethane and methanol as eluents in a gradient starting from 100% dichloromethane and ending with 95% dichloromethane and 5% methanol. The fractions containing product were combined and the solvents were evaporated under reduced pressure at 50° C. to give Compound 485 (140 mg, 49% purity, 24% yield) which was used in the next step.

### Preparation of Compound 486

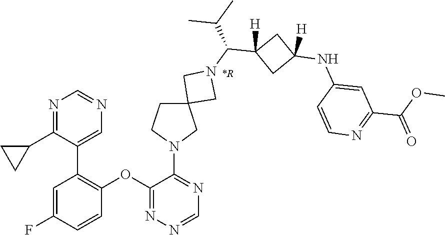

A mixture of Compound 1a (250 mg, 0.43 mmol), methyl 4-bromopicolinate (0.18 g, 0.85 mmol) and cesium carbonate (0.42 g, 1.28 mmol) in 1,4-dioxane (3 mL) was degassed with N₂. Then, palladium(II) acetate (0.0096 g, 0.043 mmol) and 4,5-bis(diphenylphosphino)-9,9-dimethylxanthene (0.025 g, 0.043 mmol) were added. The reaction mixture was purged with N₂ and heated at 100° C. for 18 h. An additional amount of methyl 4-bromopicolinate (0.092 g, 0.43 mmol), palladium(II) acetate (0.0096 g, 0.043 mmol) and 4,5-bis(diphenylphosphino)-9,9-dimethylxanthene (0.025 g, 0.043 mmol) was added. The reaction mixture was heated at 100° C. for 18 h. The reaction mixture was cooled down to room temperature, diluted with ethylacetate and then filtered. The filtrate was washed with water and brine, dried with MgSO₄, filtered and the solvents of the filtrate evaporated under reduced pressure at 45° C. The residue was dissolved in dichloromethane and purified over a SiO₂ column, 12 g, using dichloromethane and methanol as eluents in a gradient starting from 100% dichloromethane and ending with 95% dichloromethane and 5% methanol. The fractions containing product were combined and the solvents were evaporated under reduced pressure at 50° C. to give Compound 486 (27 mg, 5.5% yield).

### Preparation of Compound 488

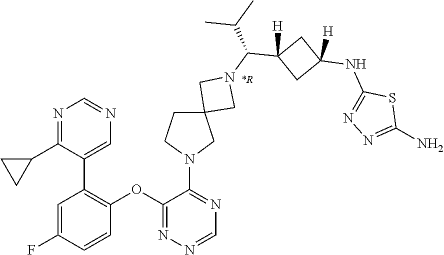

Compound 1a (150 mg, 0.275 mmol), 5-bromo-1,3,4-thiadiazol-2-amine (49.579 mg, 0.275 mmol) and DIPEA (0.237 mL, 0.75 g/mL, 1.377 mmol) were added to MeCN (15 mL, 0.786 g/mL, 287.193 mmol). The mixture was stirred at 75° C. for 2 hours. The solvent was removed and the residue was purified by flash column (C18, CH₃CN:H₂O from 0:100 to 50:50, 0.5% fumarate as buffer) to afford Compound 488 (140 mg, 75% yield).

### Preparation of Compound 489

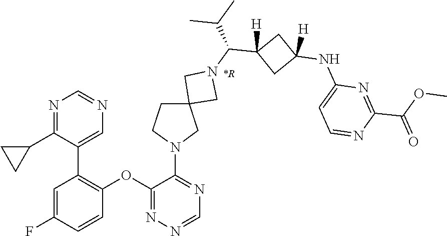

In a closed vial, Compound 1a (350 mg, 0.598 mmol) was treated with methyl 4-bromopyrimidine-2-carboxylate (155.6 mg, 0.717 mmol), Cs₂CO₃ (584.1 mg, 1.793 mmol), and JOSIPHOS SL-J009-1 Pd G3 (55.2 mg, 0.0598 mmol). Then dry DMA (6 mL) was added and the mixture was stirred for 6 h at 70° C. Then mixture was allowed to stand over the weekend at rt. Then the mixture was heated again over night. The mixture was worked up by addition of sat. sodium carbonate solution and EtOAc. The phases were separated and the water phase was extracted multiple times with EtOAc and DCM. Drying with magnesium sulfate, filtration and evaporation of solvents afforded the crude material which was subjected to FCC (silica gel, 2% to 10% methanol in DCM, evaporation of solvents at 35° C. water bath temp) to afford Compound 489 (25 mg, 6.1% yield).

### Preparation of Compound 490

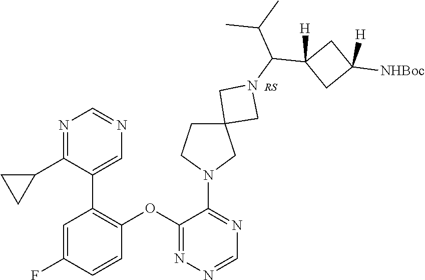

To a solution of intermediate 15 (200 mg, 0.582 mmol) in CH₂Cl₂ (12 mL) was added TEA (1.9 mL, 14 mmol). The mixture was stirred at 20° C. for 3 minutes and intermediate 8 (295 mg, 0.874 mmol) was added. The mixture was stirred at 20° C. for 1 h. The mixture was diluted with CH₂Cl₂ (20 mL) and washed with H₂O (20 mL) and brine (20 mL). The organic layer was dried over anhydrous Na₂SO₄, filtered and concentrated under reduced pressure to give crude product, which was purified by FCC (eluting with petroleum ether:ethyl acetate=100:0 to 0:100) to afford the product (250 mg, yield 66.6%) as a yellow solid.

Alternative preparation of Compound 490. The reaction was performed twice on 5 g of intermediate 33. The resulting crude mixtures were combined for the work up and purification.

A mixture of intermediate 33 (5 g; 11.9 mmol), intermediate 6 (4.3 g; 17.8 mmol), AcOH (1.4 mL; 23.7 mmol) and NaBH₃CN (2.3 g; 35.6 mmol) in MeOH (190 mL) was stirred at 50° C. overnight. This reaction was performed twice and the two reaction mixtures were combined and poured onto a 10% aqueous solution of K₂CO₃, DCM was added. The layers were separated and then, the aqueous layer was extracted with DCM (3×). The organic layer was dried over MgSO₄, filtered and the solvent was evaporated. The crude product was purified by chromatography over silica gel (mobile phase: 96% DCM, 4% MeOH, 0.1% NH₄OH). The pure fractions were collected and the solvent was evaporated to afford 12 g (78%) of Compound 490.

### Preparation of Compound 491a & 491b

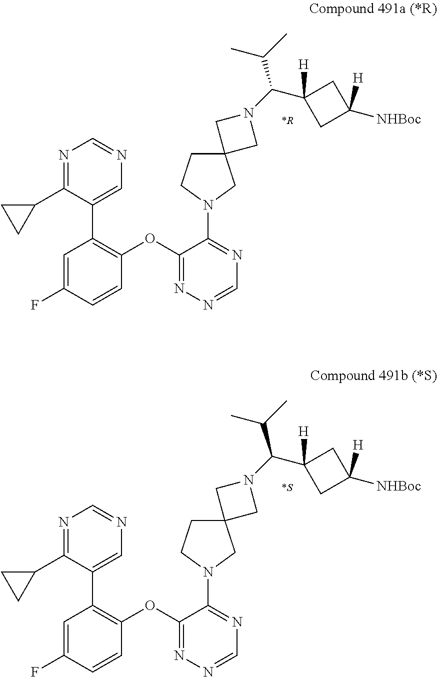

Compound 490 (230 mg, 0.357 mmol) was separated by SFC (column: DAICEL CHIRALPAK AD-H (250 mm*30 mm, 5 μm), eluent: 35% (v/v) super critical CO₂ in 0.1% NH₃H₂O IPA, flow rate: 60 mL/min) to afford Compound 491a (90 mg, 39% yield) and Compound 491b (90 mg, 39% yield) both as a yellow solid.

### Alternative Preparation of Compound 491a

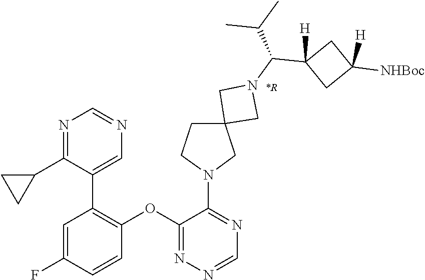

DBU (4.0 mL, 27 mmol) was added into a solution of intermediate 294 (1.60 g, 3.93 mmol) and intermediate 283 (1.94 g, 4.73 mmol) in ACN (20 mL). The reaction was stirred at RT for 2 h. The mixture was diluted with H₂O (20 mL) and extracted with DCM (30 mL×3). The combined organic layers were concentrated and purified by FCC (DCM:MeOH=1:0 to 10:1) to afford Compound 491a (1.705 g, 67% yield) as a white solid.

The compounds reported below were prepared following an analogous methodology as reported for the alternative preparation of Compound 491a starting from the corresponding intermediates:

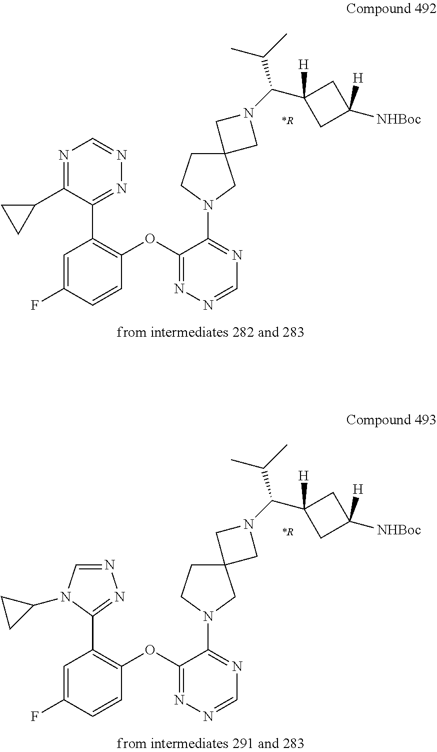

### Preparation of Compound 494

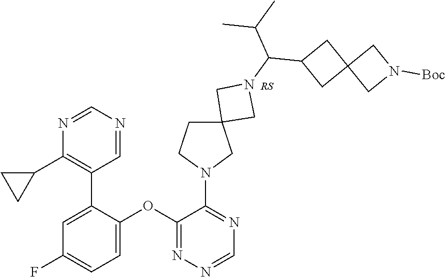

A mixture of intermediate 33 (1.78 g; 4.3 mmol), intermediate 61 (1.47 g; 5.5 mmol), AcOH (242 μL; 4.3 mmol) and NaBH₃CN (798 mg; 12.7 mmol) in MeOH (36 mL) was stirred at 50° C. overnight. The reaction mixture was poured onto a saturated solution of NaHCO₃ and DCM was added. The mixture was extracted with DCM (3×). The organic layer was dried over MgSO₄, filtered and the solvent was evaporated. The crude product (3.1 g) was purified by chromatography over silica gel (Mobile phase: Gradient from 99% DCM, 1% MeOH (+10% NH₄OH) to 95% DCM, 5% MeOH (+10% NH₄OH)). The pure fractions were collected and the solvent was evaporated to give 1.95 g (70%) of Compound 494.

The intermediate reported below was prepared following an analogous methodology as described for Compound 494 starting from the corresponding intermediates:

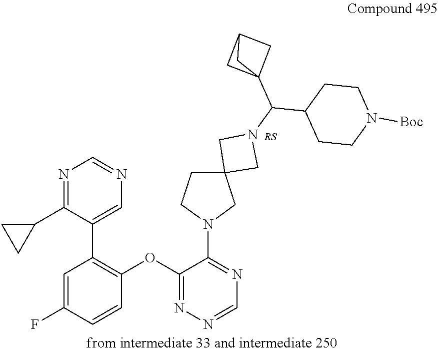

### Preparation of Compound 496 (RS), 496a (*R) and 496b (*S

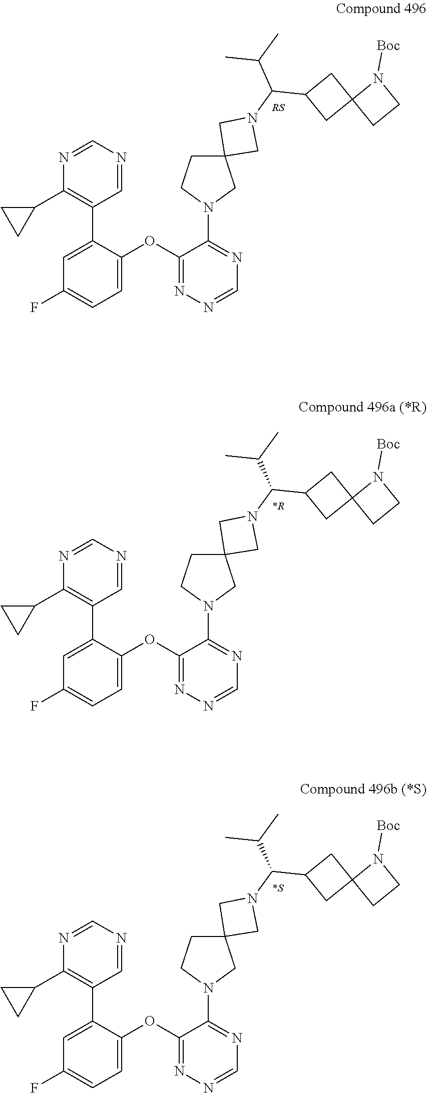 [SMILES: CC(C)C(C1CC2(CCN2C(=O)OC(C)(C)C)C1)N1CC2(CCN(c3ncnnc3Oc3ccc(F)cc3-c3cncnc3C3CC3)C2)C1 ; CC(C)[C@H](C1CC2(CCN2C(=O)OC(C)(C)C)C1)N1CC2(CCN(c3ncnnc3Oc3ccc(F)cc3-c3cncnc3C3CC3)C2)C1 ; CC(C)[C@H](C1CC2(CCN2C(=O)OC(C)(C)C)C1)N1CC2(CCN(c3ncnnc3Oc3ccc(F)cc3-c3cncnc3C3CC3)C2)C1]

A mixture of intermediate 33 (367 mg; 0.9 mmol), intermediate 62 (304 mg; 1.2 mmol), AcOH (50 μL; 0.9 mmol) and NaBH₃CN (165 mg; 2.7 mmol) in MeOH (8 mL) was stirred at 50° C. overnight. The reaction mixture was poured onto a saturated solution of NaHCO₃ and DCM was added. The mixture was extracted with DCM (3×). The organic layer was dried over MgSO₄, filtered and the solvent was evaporated. The crude product (614 mg) was purified by chromatography over silica gel (Mobile phase: Gradient from 99% DCM, 1% MeOH (+10% NH₄OH) to 95% DCM, 5% MeOH (+10% NH₄OH)). The pure fractions were collected and the solvent was evaporated. The Compound 496 (420 mg, 72%) was purified by chiral SFC (Stationary phase: CHIRALPAK AD-H 5 μm 250*30 mm, Mobile phase: 80% CO₂, 20% iPrOH (+0.3% iPrNH₂)). The pure fractions were collected and the solvent was evaporated till dryness to give 143 mg (24%) of Compound 496a (*R) and 147 mg (25%) of Compound 496b (*S).

### Preparation of Compound 497 and Compound 498

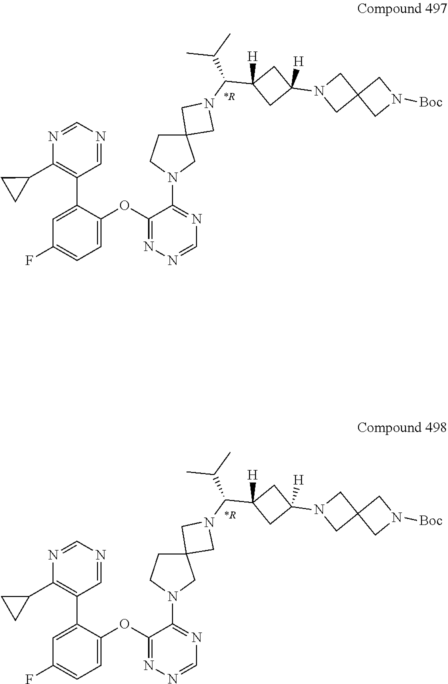 [SMILES: CC(C)[C@H]([C@H]1C[C@@H](N2CC3(CN(C(=O)OC(C)(C)C)C3)C2)C1)N1CC2(CCN(c3ncnnc3Oc3ccc(F)cc3-c3cncnc3C3CC3)C2)C1 ; CC(C)[C@H]([C@H]1C[C@H](N2CC3(CN(C(=O)OC(C)(C)C)C3)C2)C1)N1CC2(CCN(c3ncnnc3Oc3ccc(F)cc3-c3cncnc3C3CC3)C2)C1]

NaBH₃CN (462 mg; 7.36 mmol) was added to a mixture of intermediate 38b (2 g; 3.68 mmol), 2,6-diazaspiro[3.3]heptane-2-carboxylic acid tert-butyl ester hemioxalate (2.24 g; 9.20 mmol) and acetic acid (211 μL; 3.68 mmol) in MeOH (100 mL) and the reaction mixture was stirred at 60° C. for 18 h. The reaction mixture was cooled to rt, poured onto a 10% aqueous solution of K₂CO₃ and DCM. The mixture was filtered through Chromabond® and the filtrate was evaporated to dryness. The residue (5 g) was purified by chromatography over silica gel (irregular SiOH, 80 g; mobile phase: 0.7% NH₄OH, 93% DCM, 7% MeOH). The pure fractions were collected and evaporated to dryness. Then, The residue (2.1 g) was purified by reverse phase chromatography (YMC-actus Triart C18 10 μm 30*150 mm; mobile phase: gradient from 40% NH₄HCO₃ 0.2% pH=9.5, 30% MeOH, 30% ACN to 10% NH₄HCO₃ 0.2% pH=9.5, 45% MeOH, 45% ACN). The pure fractions were collected and evaporated to dryness yielding 930 mg of Compound 497 (35%) as a white foam and 750 mg of Compound 498 (28%) as a white foam.

### Preparation of Compound 499

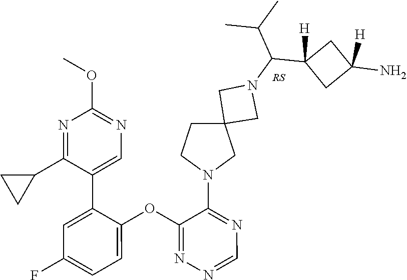 [SMILES: COc1ncc(-c2cc(F)ccc2Oc2nncnc2N2CCC3(C2)CN(C(C(C)C)[C@H]2C[C@@H](N)C2)C3)c(C2CC2)n1]

To a solution of Compound 501 (1.0 g, 1.48 mmol) in DCM (30 mL) was added TFA (10 mL) at 0° C. The resulting solution was stirred for 1 h at room temperature. The resulting mixture was concentrated under reduce pressure. The residue was diluted with water, and then, the pH was adjusted to 9 with NaOH solution (1 M in water). The resulting solution was extracted with DCM. The combined organic layers were dried over anhydrous Na₂SO₄, filtered and concentrated under reduced pressure to give 860 mg (92%) of the desired Compound 499 as a white solid.

### Preparation of Compound 500

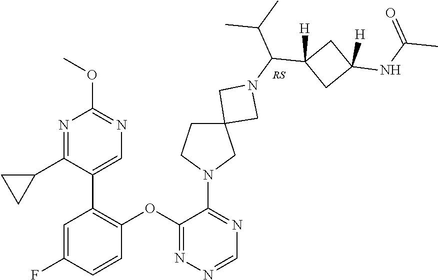 [SMILES: COc1ncc(-c2cc(F)ccc2Oc2nncnc2N2CCC3(C2)CN(C(C(C)C)[C@H]2C[C@@H](NC(C)=O)C2)C3)c(C2CC2)n1]

To a solution of Compound 499 (210 mg, 0.37 mmol) in DCM (8 mL) were added Et₃N (0.8 mL) and acetic anhydride (0.8 mL) at 0° C. The resulting mixture was stirred for 1 h at room temperature. The resulting mixture was quenched with NaHCO₃ aqueous solution and extracted with DCM. The combined organic layers were washed with brine and dried over sodium sulfate, filtered and concentrated under reduced pressure. The resulting crude product was purified by prep-HPLC (Column: XBridge C18 OBD Prep Column, 100Å, 5 μm, 19 mm×250 mm; Mobile Phase A: Water (10 MMOL/L NH₄HCO₃+0.1% NH₃·H₂O), Mobile Phase B: ACN; Flow rate: 25 mL/min; Gradient: 40% B to 50% B). The pure fractions were collected and the solvent was evaporated. Then, the resulting residue was lyophilized to give 50.3 mg (21%) of the desired Compound 500 as a white solid.

### Preparation of Compound 501

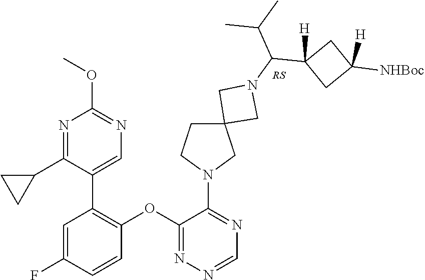 [SMILES: COc1ncc(-c2cc(F)ccc2Oc2nncnc2N2CCC3(C2)CN(C(C(C)C)[C@H]2C[C@@H](NC(=O)OC(C)(C)C)C2)C3)c(C2CC2)n1]

To a solution of intermediate 118 (1.0 g, 2.23 mmol) in MeOH (40 mL) was added intermediate 6 (1.1 g, 4.45 mmol). After stirring for 30 minutes at room temperature, NaBH₃CN (700 mg, 11.12 mmol) was added to the reaction mixture. The resulting mixture was stirred at 50° C. overnight, cooled to room temperature, quenched with water and extracted with EtOAc. The combined organic layers were washed with brine and dried over sodium sulfate, filtered and concentrated under reduced pressure. The residue was purified by flash chromatography over silica gel (MeOH/DCM: 1/10). The pure fractions were collected and evaporated to dryness to give 1.05 g (64%) of desired Compound 501 as a white solid.

### Preparation of Compound 502 (N,N-dimethyl-3-[(1R)-1-[7-[6-[2-(3-cyclopropyl-5-methyl-1,2,4-triazol-4-yl)-4-fluoro-phenoxy]-1,2,4-triazin-5-yl]-2,7-diazaspiro[3.4]octan-2-yl]-2-methyl-propyl]cyclobutanamine

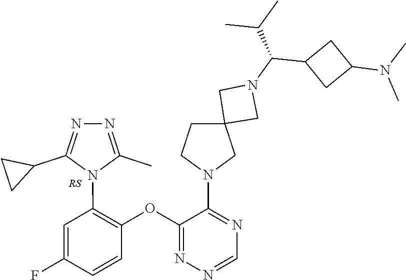 [SMILES: Cc1nnc(C2CC2)n1-c1cc(F)ccc1Oc1nncnc1N1CCC2(C1)CN([C@H](C(C)C)C1CC(N(C)C)C1)C2]

Sodium cyanoborohydride (25 mg, 0.384 mmol) was added to 3-[rac-(1R)-1-[7-[6-[2-(3-cyclopropyl-5-methyl-1,2,4-triazol-4-yl)-4-fluoro-phenoxy]-1,2,4-triazin-5-yl]-2,7-diazaspiro[3.4]octan-2-yl]-2-methyl-propyl]cyclobutanone (105 mg, 0.192 mmol), dimethyl amine (2 M in THF, 0.48 mL, 0.96 mmol) and acetic acid (11 μL, 0.192 mmol) in MeOH (6 mL) and the reaction mixture was stirred at ambient temperature for 2 h. The reaction mixture was poured onto a 10% aqueous solution of K₂CO₃ and dichloromethane. The mixture was extracted with dichloromethane (3×20 mL). The organic layer was dried over MgSO₄, filtered and the solvent was evaporated to give N,N-dimethyl-3-[rac-(1R)-1-[7-[6-[2-(3-cyclopropyl-5-methyl-1,2,4-triazol-4-yl)-4-fluoro-phenoxy]-1,2,4-triazin-5-yl]-2,7-diazaspiro[3.4]octan-2-yl]-2-methyl-propyl]cyclobutanamine (98 mg) as a mixture of atropisomers.

### Preparation of Compound 503 (N,N-dimethyl-3-[(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutanamine

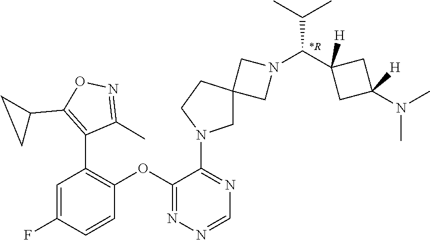 [SMILES: Cc1noc(C2CC2)c1-c1cc(F)ccc1Oc1nncnc1N1CCC2(C1)CN([C@H](C(C)C)[C@H]1C[C@@H](N(C)C)C1)C2]

Sodium cyanoborohydride (56 mg, 0.878 mmol) was added to a mixture of 3-[rac-(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutanone (240 mg, 0.439 mmol), dimethyl amine (1.1 mL, 2 M in THF, 2.195 mmol) and acetic acid (26 μL, 0.439 mmol) in MeOH (15 mL) and the reaction mixture was stirred at ambient temperature for 2 h. The reaction mixture was then poured onto a 10% aqueous solution of K₂CO₃ and dichloromethane. The mixture was extracted with dichloromethane (3 x). Combined organic layer was washed with brine, dried over anhydrous MgSO₄, filtered and the solvent was evaporated. The residue was purified by flash column chromatography (0 to 2% 7N MeOH—NH₃ in dichloromethane as eluents) to give N,N-dimethyl-3-[rac-(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutanamine (14 mg, 16%).

### Preparation of Compound 506 (tert-butyl N-[3-[(1R)-1-[7-[3-chloro-6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutyl]carbamate

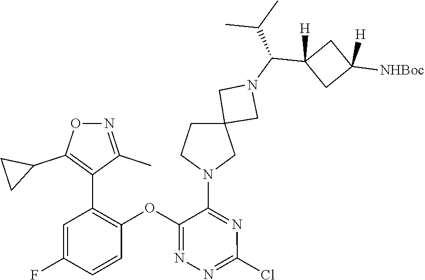 [SMILES: Cc1noc(C2CC2)c1-c1cc(F)ccc1Oc1nnc(Cl)nc1N1CCC2(C1)CN([C@H](C(C)C)[C@H]1C[C@@H](NC(=O)OC(C)(C)C)C1)C2]

A mixture of intermediate 333 (230 mg, 0.474 mmol), 2-(5-cyclopropyl-3-methyl-isoxazol-4-yl)-4-fluoro-phenol (111 mg, 0.474 mmol) and Cs₂CO₃ (186 mg, 0.569 mmol) in anhydrous DMF (10 mL) was stirred at RT for 18 h. Upon completion, reaction mixture was diluted with dichloromethane (30 mL) and washed with water. Layers were separated and aqueous layer was extracted with dichloromethane (2×25 mL). Combined organic layer was washed with water, brine, dried over anhydrous MgSO₄ and rotary evaporated. The residue was purified by flash column chromatography (0 to 2% MeOH in dichloromethane) to give Compound 506 (225 mg, yield 69%).

### Preparation of Compound 504 (tert-butyl N-[3-[(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutyl]carbamate

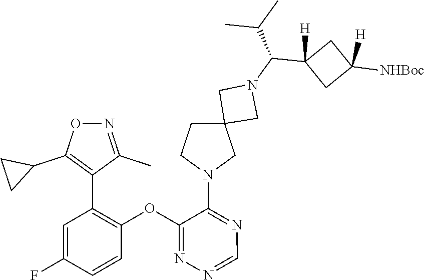 [SMILES: Cc1noc(C2CC2)c1-c1cc(F)ccc1Oc1nncnc1N1CCC2(C1)CN([C@H](C(C)C)[C@H]1C[C@@H](NC(=O)OC(C)(C)C)C1)C2]

Pd/C (10%) (25 mg) was added to a solution of Compound 506 (160 mg, 0.235 mmol) and thiophene (0.06 mL, 0.4 M, 0.0235 mmol) in MeOH (20 mL) at ambient temperature and the mixture was stirred under H₂ (1 atm) for 1 h. Upon completion (LCMS), the mixture was filtered over dicalite and the solvent was evaporated under vacuum. The residue was purified by flash column chromatography (0 to 2% 7N MeOH—NH₃ in dichloromethane as eluents) to give Compound 504 (40 mg, 26%).

### Preparation of Compound 507 (tert-butyl N-[3-[(1R)-1-[7-[3-chloro-6-[2-(3-cyclopropyl-5-methyl-1,2,4-triazol-4-yl)-4-fluoro-phenoxy]-1,2,4-triazin-5-yl]-2,7-diazaspiro[3.4]octan-2-yl]-2-methyl-propyl]cyclobutyl]carbamate

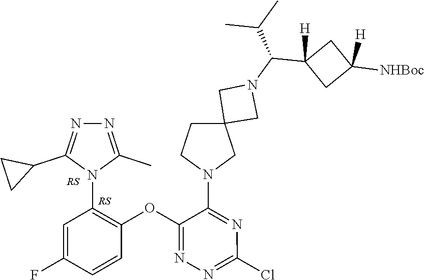 [SMILES: Cc1nnc(C2CC2)n1-c1cc(F)ccc1Oc1nnc(Cl)nc1N1CCC2(C1)CN([C@H](C(C)C)[C@H]1C[C@@H](NC(=O)OC(C)(C)C)C1)C2]

A mixture of intermediate 333 (1 g, 2.06 mmol), Intermediate 345 (481 mg, 2.06 mmol) and Cs₂CO₃ (806 mg, 2.472 mmol) in anhydrous DMF (60 mL) was stirred at RT for 18 h. Upon completion, reaction mixture concentrated to dryness and diluted with dichloromethane (100 mL) and washed with water. Layers were separated and aqueous layer was extracted with dichloromethane (2×50 mL). Combined organic layer was washed with water, brine, dried over anhydrous MgSO₄ and rotary evaporated. The residue was purified by flash column chromatography (0 to 2% MeOH in dichloromethane) to afford Compound 507 (1 g, yield 71%).

### Preparation of Compound 505 (tert-butyl N-[3-[(1R)-1-[7-[6-[2-(5-cyclopropyl-3-methyl-isoxazol-4-yl)-4-fluoro-phenoxy]-1,2,4-triazin-5-yl]-2,7-diazaspiro[3.4]octan-2-yl]-2-methyl-propyl]cyclobutyl]carbamate

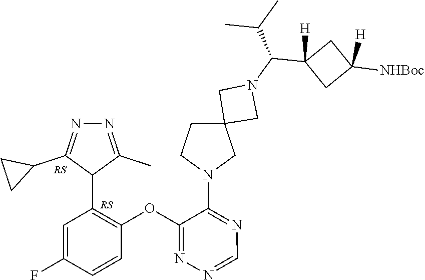 [SMILES: CC1=NN=C(C2CC2)C1c1cc(F)ccc1Oc1nncnc1N1CCC2(C1)CN([C@H](C(C)C)[C@H]1C[C@@H](NC(=O)OC(C)(C)C)C1)C2]

Pd/C (10%) (141 mg) was added to a solution of Compound 507 (900 mg, 1.319 mmol) and thiophene (0.33 mL, 0.4 M, 0.132 mmol) in MeOH (100 mL) at ambient temperature and the mixture was stirred under H₂ (1 atm) for 1 h. Upon completion (LCMS), the mixture was filtered over dicalite and the solvent was evaporated under vacuum. The residue was purified by flash column chromatography (0 to 3% MeOH—NH₃ in dichloromethane as eluents) to give Compound 505 (420 mg, 49%).

### Analytical Methods

The analytical information in the Examples above or in the Tables below, was generated by using the analytical methods described below.

### NMR-Methods

Some NMR experiments were carried out using a Bruker Avance III 400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (6) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (6) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (6) are reported in parts per million (ppm). J values are expressed in Hz.

### LCMS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE la
LCMS Method codes (Flow expressed in mL/min; column temperature (T) in °
C.; Run time in minutes). “TFA” means trifluoroacetic acid; “FA” means formic acid

| | | | | | Flow- | |
|---|---|---|---|---|---|---|
| | | | | | (ml/mn) | |
| | | | | | ---- | Run |
| Method | | | | | Column | time |
| code | Instrument | Col μmn | Mobile phase | Gradient | T (° C.) | (min) |
| 1 | Waters: | Waters: BEHA: | A: 95% | 84.2% A for 0.49 min, to | 0.343 | 6.2 |
| | Acquity | C18 (1.7 μm, | CH₃COONH₄ | 10.5% A in 2.18 min, held | 40 | |
| | UPLC ® - | 2.1 × 100 mm) | 7 mM/5% | for 1.94 min, back to 84.2% | | |
| | DAD and | | CH₃CN, B: | A in 0.73 min, held for | | |
| | Quattro | | CH₃CN | 0.73 min. | | |
| | Micro ™ | | | | | |
| 2 | Agilent | Xtimate C18 | A: water(4 L) + | A gradient from 100% A to | 0.8 | 7 |
| | | 2.1*30 mm, 3 | TFA(1.5 mL); | 40% A is applied in 6 | 50 | |
| | | μm | mobile phase, B: | minutes, and hold at these | | |
| | | | acetonitrile | conditions for 0.5 minutes, | | |
| | | | (4 L) + | to 100% A and 0% B in | | |
| | | | TFA (0.75 mL) | 0.01 minutes and | | |
| | | | | reequilibrate with 100% A | | |
| | | | | for 0.49 minutes. A | | |
| 3 | Agilent | Xbrige | A: water(1 L)+ | From 90% A to 20% A, | 0.8 | 7 |
| | | Shield RP- | NH₃H₂O | 80% B in 6 minutes, and | 30 | |
| | | 18, 5 μm, 2.1* | (0.2 mL); mobile | hold at these conditions for | | |
| | | 50 mm | phase B: acetonitrile | 0.5 minutes, to 90% A and | | |
| | | | | 10% B in 0.01 minutes and | | |
| | | | | reequilibrate with 90% A | | |
| | | | | for 0.49 minutes | | |
| 4 | Agilent | Waters | mobile phase A: | First, 100% A was hold for | 0.8 | 10 |
| | | XBridge | water with | 1 minute. Then a gradient | 40 | |
| | | Shield RP18 | 0.05% NH₃•H₂O; | was applied to 40% A and | | |
| | | column | mobile phase | 60% B in 4 minutes and | | |
| | | (50*2.1 mm | B: | then to 5% A and 95% B in | | |
| | | 5 μm) | acetonitrile | 2.5 minutes. Finally return | | |
| | | | | to 100% A in 2 minutes and | | |
| | | | | hold for 0.5 minute. Post | | |
| | | | | Time is 0.5 minute | | |
| 5 | Shimadzu | Luna Qmega | ACN-Water- | 0.0 min 5% B −> 2.8 min | 1.5 | 4.50 |
| | LCMS-2020 | | 0.1% FA | 40% B −> 3.6 min 95% B −> | 40 | |
| | | | | 4.1 min 95% B | | |
| 6 | Shimadzu | Poroshell | ACN-Water- | 0.0 min 5% B −> 2.0 min | 1.2 | 3.00 |
| | LCMS-2020 | HPH-C18 | 0.03% NH₃H₂O | 95% B −> 2.7 min 95% B | 140 | |
| 7 | Shimadzu | Poroshell | ACN-Water- | 0.0 min 5% B −> 2.0 min | 1.2 | 3.00 |
| | LCMS-2020 | HPH-C18 | 5 mM NH₄HCO₃ | 95% B −> 2.7 min 95% B | 40 | |
| 8 | Shimadzu | Luna Omega | ACN-Water- | 0.0 min 5% B −> 2.0 min | 1.5 | 3.00 |
| | LCMS-2020 | | 0.1% FA | 95% B −> 2.7 min 95% B | 40 | |
| 9 | Shimadzu | Kinetex | ACN-Water- | 0.0 min 5% B −> 2.0 min | 1.2 | 3.00 |
| | LCMS-2020 | EVO | 5 mM NH₄HCO₃ | 95% B −> 2.7 min 95% B | 40 | |
| 10 | Shimadzu | Poroshell | ACN-Water- | 0.0 min 5% B −> 2.0 min | 1.2 | 3.00 |
| | LCMS- | | 0.04% | | | |
| | 2020 | HPH-C18 | NH₃H₂O | 95% B −> 2.7 min 95% B | 40 | |
| 11 | Shimadzu | Shim-pack | ACN-Water- | 0.0 min 5% B −> 2.0 min | 1.5 | 3.00 |
| | LCMS- | | | 100% B −> 2.7 min 100% | −40 | |
| | 2020 | XR-ODS | 0.05% TFA | B | | |
| 12 | Shimadzu | XBridge | ACN-Water- | 0.0 min 5% B −> 2.0 min | 1.2 | 3.00 |
| | LCMS- | BEH C18 | 5 mM | 95% B −> 2.7 min 95% B | −40 | |
| | 2020 | | NH₄HCO₃ | | | |
| 13 | Shimadzu | Poroshell | ACN-Water- | 0.0 min 30% B −> 3.0 | 1.2 | 4.50 |
| | LCMS- | HPH-C18 | 5 mM | min 70% B −> 3.2 min | 40 | |
| | 2020 | | NH₄HCO₃ | 95% B−> 4.2 min 95% B | | |
| 14 | Shimadzu | Ascentis | ACN-Water- | 0.0 min 5% B −> 2.0 min | 1.5 | 3.00 |
| | LCMS- | Express | 0.05% TFA | 100% B −> 2.7 min 100% | 40 | |
| | 2020 | C18 | | B | | |
| 15 | Shimadzu | HALO | ACN-Water- | 0.0 min 5% B −> 0.3 min | 1.2 | 3.00 |
| | LCMS- | | 0.05% TFA | 5% B −>2.2 min 100% B | 40 | |
| | 2020 | | | 2.7 min 100% B −> | | |
| 16 | Waters: | Waters: | A: 95% | From 85% A to 10% A in | 0.35 | 6.1 |
| | Acquity | BEH C18 | CH₃COONH₄ | 2.1 min, held for 2 min, | 40 | |
| | UPLC ® H- | (1.7 μm, | 7 mM/5% | back to 85% A in 0.8 min, | | |
| | Class - | 2.1 × 100 mm) | CH₃CN, B: | held for 0.7 min. | | |
| | DAD and | | CH₃CN | | | |
| | QDa | | | | | |
| 17 | Waters: | Waters | A: 95% | 95% A to 5% A in 1 min, | 0.5 | 3.3 |
| | Acquity | BEH ®-C18 | CH₃COONH₄ | held for 1.6 min, back to | 40 | |
| | UPLC ® H- | (1.7 μm, | 7 mM/5% | 95% A in 0.2 min, held for | | |
| | Class - | 2.1 × 100 mm) | CH₃CN, B: | 0.5 min. | | |
| | DAD and | | CH₃CN | | | |
| | QDa | | | | | |
| 18 | Waters: | Waters | A: 95% | From 95% A to 5% A in | 0.5 | 3.3 |
| | Acquity | BEH ®C18 | CH₃COONH₄ | 1 min, held for 1.6 min, | 40 | |
| | UPLC ® H- | (1.7 μm, | 7 mM/5% | back to 95% A in 0.2 min, | | |
| | Class - | 2.1 × 50 mm) | CH₃CN, B: | held for 0.5 min. | | |
| | DAD and | | CH₃CN | | | |
| | SQD 2 | | | | | |
| 19 | Agilent | Waters | A: water with | 100% A was hold for 1 | 0.8 | 10 |
| | | Xbridge- | 0.04% TFA; | minute, A gradient from | 50 | |
| | | C18 | mobile phase, | 100% A to 40% A is | | |
| | | 2.1*50 | B: acetonitrile | applied in 4 minutes, and | | |
| | | mm, 5 um | with 0.02% | 40% A down to 15% A in | | |
| | | | TFA | 2.5 minutes. And then | | |
| | | | | return to 100% A in 2 | | |
| | | | | minutes and hold for 0.5 | | |
| | | | | minutes. The post time is | | |
| | | | | 0.5 min. | | |
| 20 | Agilent | Waters | mobile phase | First, 90% A was hold | 0.8 | 10 |
| | | Xbridge- | A: water with | for 0.8 minute. Then a | 50 | |
| | | C18 | 0.04% TFA; | gradient was applied to | | |
| | | 2.1*50 | mobile phase | 20% A and 80% B in 3.7 | | |
| | | mm, 5 um | B: acetonitrile | minutes and hold for 3 | | |
| | | | with 0.02% | minutes. And then return | | |
| | | | TFA | to 90% A in 2 minutes | | |
| | | | | and hold for 0.5 minutes. | | |
| | | | | The post time is 0.5 min. | | |
| 21 | Agilent | XBridge | mobile phase | First, 100% A was hold | 0.8 | 10 |
| | | ShieldRP18, | A: H₂O | for 1 minute. Then a | 40 | |
| | | 50*2.1 mm | (0.05% | gradient was applied to | | |
| | | 5 μm | NH₃•H₂O); | 40% A and 60% B in 4 | | |
| | | | mobile phase | minutes and then to 5% A | | |
| | | | B: acetonitrile | and 95% B in 2.5 | | |
| | | | | minutes. 95% B was hold | | |
| | | | | for 2.0 minute. Finally | | |
| | | | | return to 100% A and | | |
| | | | | hold for 0.5 minute. Post | | |
| | | | | Time is 1.5 minute. | | |
| 22 | SHIMAD | Xbridge | mobile phase | from 90% A to 20% A, | 0.8 | 7 |
| | ZU LC20- | Shield RP- | A: | 80% B in 6 minutes, and | 50 | |
| | MS2020 | 18, 5 μm, | water(4 L) + | hold at these conditions | | |
| | | 2.1*50 mm | NH₃•H₂O | for 0.5 minutes, to 90% A | | |
| | | | (2 mL); mobile | and 10% B in 0.01 | | |
| | | | phase B: | minutes and reequilibrate | | |
| | | | acetonitrile | with 90% A for 0.49 | | |
| | | | | minutes. | | |
| 23 | SHIMAD | Xbridge | mobile phase | 10% to 80% (mobile | 1 | 3 |
| | ZU LC20- | Shield RP- | A: | phase B) over 2 minutes | 50 | |
| | MS2020 | 18, 5 μm, | water(4 L) + | and holding at 80% | | |
| | | 2.1*50 mm | NH₃•H₂O | for 0.48 minutes | | |
| | | | (0.8 mL); | | | |
| | | | mobile phase | | | |
| | | | B: | | | |
| | | | acetonitrile | | | |
| 24 | Agilent | Xtimate | mobile phase | 10% to 80% (mobile | 1.2 | 2 |
| | | C18 | A: | phase B) over 0.9 minutes | 50 | |
| | | 2.1*30 mm, | water(4 L) + | and holding at 80% for | | |
| | | 3 um | TFA (1.5 mL); | 0.6 minutes | | |
| | | | mobile phase | | | |
| | | | B: | | | |
| | | | acetonitrile | | | |
| | | | (4 L) + TFA | | | |
| | | | (0.75 mL) | | | |
| 25 | Agilent | Waters: | A: H₂O 0.1% | 95% A to 5% A in | 1 | 3.5 |
| | 6120 | Sunfire | FA, B: ACN | 2.5 min, held for 1.0 | 40 | |
| | LC/MS | C18 | | min | | |
| | | (2.5 μm, | | | | |
| | | 3.0 × 30 | | | | |
| | | mm) | | | | |
| 26 | Shimadzu | Shim- | A: H₂O | 5% B to 100% B in | 1.5 | 2 |
| | LCMS20 | pack | 0.05% TFA | 1.1 min, hold for 0.6 | 40 | |
| | 20, | Velox | B: ACN | min. Then, to 5% B in | | |
| | LC30AD | SP-C18 | 0.05% TFA | 0.3 mn | | |
| | | (3.0*30 | | | | |
| | | mm, 2.7 | | | | |
| | | u) | | | | |
| 27 | Shimadzu | Shim- | A: H₂O | 2% B to 100% B in 2 | 0.5 | 3 |
| | LCMS20 | pack | 0.05% TFA | min, then 100% B for | 40 | |
| | 20, | Velox | B: ACN | 0.7 m. then to 2% B in | | |
| | LC30AD | SP-C18 | 0.05% TFA | 0.3 mn, | | |
| | | (3.0*30 | | | | |
| | | mm, 2.7μ) | | | | |
| 28 | Shimadzu | Shim- | A: H₂O | 2% B to 100% B in 2 | 0.5 | 3 |
| | LCMS20 | pack | 0.05% TFA | min, then 100% B for | 40 | |
| | 20, | Velox | B: ACN | 0.7 mn then to 5% B in | | |
| | LC30AD | SP-C18 | 0.05% TFA | 0.3 mn | | |
| | | (3.0*30 | | | | |
| | | mm, 2.7μ) | | | | |
| 29 | SHIMAD | Xtimate | mobile phase | a gradient condition | 0.8 | 7 |
| | ZU | C18 | A: | from 100% A to 40% | 50 | |
| | LC20- | 2.1*30 mm, | A: water(4 L) + | A, 60% B in 6 minutes, | | |
| | MS2010 | 3 um | TFA(1.5 mL) | and hold at these | | |
| | | | mobile phase | conditions for 0.5 | | |
| | | | B: | minutes, then to 100% | | |
| | | | B: acetonitrile | A and 0% B in 0.1 min | | |
| | | | (4 L) + | and held for 0.49 min. | | |
| | | | TFA(0.75 mL) | | | |
| 30 | Shimadzu | kinetex | ACN-Water- | 0.0 min 10% B −> 2.0 | 1.2 | 3.00 |
| | LCMS- | EVO-C18 | 6.5 mM | min 95% B −>2.7 min | 40 | |
| | 2020 | | NH₄HCO₃+ | 95% B −> 2.75 min | | |
| | | | NH₃H₂O | 10% B−>3.00 min | | |
| | | | | Controller | | |
| 31 | Shimadzu | Poroshell | ACN-Water- | 0.0 min 10% B −> 2.0 | 1.2 | 2.85 |
| | LCMS- | HPH C18 | 5 mM | min 95% B −> 2.7 min | 40 | |
| | 2020 | | NH₄HCO₃ | 95% B−> 2.75 min 10% | | |
| | | | | B−> 2.85 min | | |
| | | | | Controller | | |
| 32 | Agilent | Agilent | mobile phase | A gradient condition | 1 | 3 |
| | | Poroshell | A: | from 95% A to 20% | 50 | |
| | | 120 EC- | A: water(4 L) + | A, 80% B in 1.2 | | |
| | | C18 | TFA(1.5 mL) | minutes, then to 5% A | | |
| | | 1.9 um | mobile phase | and 95% B in 1.3 min. | | |
| | | 3.0*30 mm | B: | Then return to 95% A in | | |
| | | | B: acetonitrile | 0.1 min and held for | | |
| | | | (4 L) + TFA(0.75 | 0.49 min. | | |
| | | | mL) | | | |
| 33 | Waters: | Waters: | A: 10 mM | From 95% A to | 0.8 | 2 |
| | Acquity ® | BEH C18 | CH₃COONH₄ | 5% A in 1.3 min, | 55 | |
| | UPLC ® - | (1.7 μm, | in 95% H₂O + | held for 0.7 min | | |
| | DAD and | 2.1*50 mm) | 5% CH₃CN | | | |
| | SQD | | B: CH₃CN | | | |
| 34 | Waters: | Waters: | A: 10 mM | From 100% A to | 0.6 | 3.5 |
| | Acquity ® | BEH | CH₃COONH₄ | 5% A in 2.10 min, | 55 | |
| | UPLC ® - | (1.8 μm, | in 95% H₂O + | to 0% A in 0.9 min | | |
| | DAD and | 2.1*100 mm) | 5% CH₃CN | | | |
| | SQD | | B: CH3CN | | | |
| 35 | Waters: | Waters: | A: 0.1% | From 100% A to | 0.8 | 2.0 |
| | Acquity ® | BEH | NH₄HCO₃ | 5% A in 1.3 min, | 55 | |
| | UPLC ® - | (1.8 μm, | in 95% H₂O + | hold 0.7 min | | |
| | DAD and | 2.1*50 mm) | 5% CH₃CN | | | |
| | SQD | | B: CH₃CN | | | |
| 36 | Waters: | Waters: | A: 10 mM | From 100% A to | 0.6 | 3.5 |
| | Acquity ® | BEH | NH₄HCO₃ | 5% A in 2.10 min, | 55 | |
| | UPLC ® - | (1.8 μm, | in 95% H₂O + | to 0% A in 0.9 min | | |
| | DAD and | 2.1*100 mm) | 5% CH₃CN | | | |
| | SQD | | B: CH₃CN | | | |
| 37 | Agilent: | Waters: | A: 0.1% FA | Gradient start from 5% | 1.2 | 3.5 |
| | 1260 | Sunfire | solution in | of B increase to 95% | −50 | |
| | Infinity | C18 | water | within 2.5 min and keep | | |
| | and 6120 | (2.5 μm, | B: CH₃CN | at 95% till 3.5 min | | |
| | Quadrupole | 3.0 × 30 mm) | | | | |
| | LC/MS | | | | | |
| 38 | Waters: | Waters: | A: 10 mM | From 100% A to | 0.6 | 3.5 |
| | Acquity ® | BEH | CH₃COONH₄ | 5% A in 2.00 min, | 55 | |
| | UPLC ® -DAD | (1.8 μm, | in 95% H₂O + | to 0% A in0.9 min, | | |
| | and SQD | 2.1*100 mm) | 5% CH₃CN | | | |
| | | | B: CH₃CN | | | |
| 39 | Shimadzu | Luna | ACN-Water- | 0.0 min 5% B −> 2.0 | 1.5 | 3.0 |
| | LCMS- | Omega | 0.1% FA | min 95% B −>2.6 min | 40 | |
| | 2020 | | | 95% B −> 2.7 min 5% | | |
| | | | | B −> 3.00 min Controller | | |
| 40 | Waters: | Water: s | A: 10 mM | From 100% A to | 0.6 | 3.5 |
| | Acquity ® | BEH | CH₃COONH₄ | 5% A in 2.10 min, | 55 | |
| | UPLC ® - | (1.7 μm, | in 95% H₂O + | to 0% A in 0.9 min, | | |
| | DAD and | 2.1*100 mm) | 5% CH₃CN | to 5% A in 0.5 min | | |
| | SQD | | B: CH₃CN | | | |
| 41 | Shimadzu | MERCK, | A: water(4 L) + | a gradient condition | 1.5 | 1.5 |
| | LC20- | RP-18e | TFA(1.5 mL) | from 95% A, 5% B to | 50 | |
| | MS2010 | 25-2 mm | B: acetonitrile | 5% A, 95% B in 0.7 | | |
| | | | (4 L) + TFA(0.75 | minutes, hold at these | | |
| | | | mL) | conditions for 0.4 | | |
| | | | | minutes, and then to | | |
| | | | | 95% A, 5% B in 0.01 | | |
| | | | | min and held for 0.49 | | |
| | | | | min. | | |
| 42 | Waters: | Waters | A: | From 95% A/5% B to 5% A | 0.5 | 3.5 |
| | Acquity ® | BEH ® | CH₃COONH₄ | in 1 min, held for 1.6 min, back | −40 | |
| | H-Class - | C18 | 7 mM 95%/ | to 95% A/5% B in 0.2 min, | | |
| | DAD and | (1.7 μm, | CH₃CN 5%, B: | held for 0.5 min. | | |
| | SQD2 ™ | 2.1 × 50 mm) | CH₃CN | | | |
| 43 | Shimadzu | Kinetex | A: Water- | 95% A to 5% A in 1.10 | 1.2 | 1.80 |
| | LCMS- | EVO | 5 mM | min, held for 0.50 min, | −40 | |
| | 2020 | (2.6 μm, | NH₄HCO₃, | back to 95% A in 0.05 | | |
| | | 3.0 × 50 | B: ACN | min, held for 0.15 min | | |
| | | mm) | | | | |
| 44 | Shimadzu | Ascentis | A: Water- | 95% A to 5% A in 1.20 | 1.5 | 3.00 |
| | LCMS- | Express | 0.05% TFA, | min, held for 1.50 min, | −40 | |
| | 2020 | C18 | B: ACN | | | |
| | | (2.7 μm, | | back to 95% A in 0.1 | | |
| | | 3.0 × 50 | | min, held for 0.20 min | | |
| | | mm) | | | | |
| 45 | Shimadzu | Xtimate | A: | a gradient condition | 1 | 2 |
| | LCMS- | C18 | water(4 L) + | from 100% A, 00% B | 50 | |
| | 2020 | 2.1*30 mm, | TFA(1.5 mL) | to 40% A, 60% B in 0.9 | | |
| | | 3 um | B: | minutes, hold at these | | |
| | | | acetonitrile(4 | conditions for 0.6 | | |
| | | | L) + TFA(0.75 | minutes, and then to | | |
| | | | mL | 100% A, 00% B in 0.01 | | |
| | | | | min and held for 0.49 | | |
| | | | | min. | | |

TABLE 1b
LCMS and melting point data. Co. No. means compound
number; Rₜ means retention time in min.

| Co. No. | Rt | [M + H]⁺ | [M + CH₃COO]⁻ | LCMS |
|---|---|---|---|---|
| 6 | 2.46 | 629.6 | 687.7 | 1 |
| 4 | 2.44 | 629.4 | | 2 |
| 5 | 4.24 | 629.4 | | 3 |
| 2 | 4.13 | 587.2 | | 4 |
| 7 | 2.25 | 573.7 | 631.8 | 1 |
| 8 | 2.39 | 573.1 | 631.7 | 1 |
| 9 | 2.39 | 691.8 | 749.9 | 1 |
| 10 | 2.30 | 647.7 | 705.6 | 1 |
| 12 | 2.24 | 629.8 | 687.8 | 1 |
| 14 | 2.42 | 641.8 | 699.8 | 1 |
| 16 | 2.46 | 643.7 | 701.7 | 1 |
| 18 | 2.30 | 599.7 | 657.6 | 1 |
| 20 | 2.28 | 627.8 | 685.7 | 1 |
| 21 | 2.43 | 627.9 | 685.8 | 1 |
| 22 | 2.22 | 629.7 | 687.8 | 1 |
| 23 | 2.59 | 690.8 | 748.8 | 1 |
| 25 | 2.30 | 690.4 | 748.5 | 1 |
| 26 | 2.29 | 690.4 | 748.5 | 1 |
| 163 | 2.26 | 629.6 | 687.5 | 1 |
| 165 | 2.26 | 629.6 | 687.8 | 1 |
| 200 | 2.92 | 631.7 | 690.0 | 1 |
| 87 | 2.34 | 643.7 | 701.9 | 1 |
| 201 | 2.32 | 643.8 | 701.8 | 1 |
| 206 | 2.91 | 631.7 | 689.7 | 1 |
| 86 | 2.28 | 647.8 | 705.6 | 1 |
| 85 | 2.27 | 647.8 | 706.2 | 1 |
| 27 | 2.54 | 612.7 | 670.7 | 1 |
| 207 | 2.37 | 663.9 | 705.7 | 1 |
| 145 | 2.35 | 627.8 | 685.7 | 1 |
| 146 | 2.49 | 627.7 | 685.8 | 1 |
| 147 | 2.35 | 571.3 | 630.4 | 1 |
| 153 | 2.36 | 572.7 | 630.3 | 1 |
| 33 | 2.31 | 647.7 | 706.5 | 1 |
| 30 | 2.36 | 647.6 | 705.8 | 1 |
| 31 | 2.43 | 647.7 | 705.7 | 1 |
| 148 | 2.63 | 584.7 | 642.7 | 1 |
| 149 | 2.51 | 640.7 | 698.7 | 1 |
| 151 | 2.38 | 626.7 | 684.4 | 1 |
| 152 | 2.53 | 626.7 | 684.7 | 1 |
| 150 | 2.63 | 640.7 | 698.7 | 1 |
| 161 | 2.51 | 684.8 | 742.7 | 1 |
| 162 | 2.55 | 684.8 | 742.7 | 1 |
| 159 | 2.40 | 560.5 | 618.5 | 1 |
| 160 | 2.40 | 560.5 | 618.6 | 1 |
| 154 | 2.32 | 641.7 | 699.7 | 1 |
| 157 | 2.34 | 641.7 | 699.8 | 1 |
| 155 | 2.36 | 641.6 | 699.7 | 1 |
| 158 | 2.36 | 641.6 | 699.7 | 1 |
| 156 | 2.43 | 641.6 | 699.7 | 1 |
| 198 | 2.24 | 571.5 | 629.6 | 1 |
| 198a | 1.02 | 571.4 | 629.3 | 17 |
| 135 | 2.45 | 613.8 | 671.8 | 1 |
| 136 | 2.30 | 573.7 | 631.9 | 1 |
| 208 | 2.54 | 615.7 | 673.8 | 1 |
| 209 | 2.66 | 719.9 | 778.8 | 1 |
| 210 | 2.32 | 670.8 | 728.8 | 1 |
| 2a | 2.29 | 587.6 | 645.7 | 1 |
| 169 | 2.66 | 681.7 | 739.7 | 1 |
| 166 | 2.76 | 666.7 | 724.7 | 1 |
| 167 | 2.75 | 666.7 | 724.8 | 1 |
| 168 | 2.76 | 666.7 | 724.6 | 1 |
| 211 | 2.27 | 629.4 | | 16 |
| 34 | 2.17 | 656.4 | 714.6 | 16 |
| 35 | 2.27 | 656.4 | 714.6 | 16 |
| 36 | 2.48 | 685.8 | 743.8 | 1 |
| 37 | 2.62 | 685.8 | 743.8 | 1 |
| 212 | 2.49 | 641.7 | 699.6 | 1 |
| 106 | 2.14 | 627.7 | 685.8 | 1 |
| 213 | 2.50 | 655.6 | 713.6 | 1 |
| 214 | 2.63 | 655.6 | 713.6 | 1 |
| 215 | 2.63 | 655.6 | 713.6 | 1 |
| 164 | 2.38 | 643.7 | 701.8 | 1 |
| 192 | 1.884 | 623.40 | | 5 |
| 193 | 1.600 | 623.10 | | 6 |
| 195 | 1.428 | 626.45 | | 7 |
| 176 | 0.923 | 587.45 | | 8 |
| 177 | 1.456 | 587.20 | | 7 |
| 178 | 1.455 | 587.25 | | 7 |
| 179 | 1.363 | 587.25 | | 9 |
| 182 | 1.349 | 587.25 | | 7 |
| 183 | 1.350 | 587.05 | | 10 |
| 196 | 1.223 | 603.25 | | 7 |
| 184 | 0.944 | 613.50 | | 8 |
| 185 | 1.020 | 613.25 | | 1 |
| 186 | 1.027 | 613.25 | | 11 |
| 189 | 0.948 | 628.50 | | 8 |
| 190 | 1.476 | 628.35 | | 12 |
| 191 | 1.473 | 628.35 | | 12 |
| 180 | 1.354 | 587.25 | | 7 |
| 181 | 1.351 | 587.25 | | 7 |
| 194 | 0.975 | 625.45 | | 8 |
| 202 | 1.689 | 628.80 | | 13 |
| 131 | 2.657 | 684.35 | | 13 |
| 132 | 1.529 | 691.30 | | 7 |
| 203 | 1.518 | 629.30 | | 7 |
| 133 | 1.398 | 617.35 | | 7 |
| 134 | 1.475 | 617.35 | | 7 |
| 187 | 0.814 | 587.55 | | 14 |
| 188 | 0.891 | 602.50 | | 8 |
| 108 | 1.046 | 629.35 | | 7 |
| 109 | 1.083 | 629.35 | | 7 |
| 68 | 0.807 | 657.60 | | 8 |
| 69 | 1.012 | 657.35 | | 15 |
| 70 | 0.824 | 657.60 | | 8 |
| 71 | 0.835 | 657.60 | | 8 |
| 110 | 0.786 | 670.60 | | 8 |
| 111 | 0.802 | 670.60 | | 8 |
| 112 | 0.752 | 629.55 | | 8 |
| 113 | 0.782 | 629.55 | | 8 |
| 114 | 0.768 | 629.55 | | 8 |
| 115 | 0.758 | 629.55 | | 8 |
| 116 | 0.791 | 670.60 | | 8 |
| 117 | 1.443 | 670.35 | | 7 |
| 138 | 2.50 | 640.6 | 698.7 | 1 |
| 139 | 2.65 | 640.6 | 698.7 | 1 |
| 120 | 2.49 | 615.6 | 673.7 | 1 |
| 121 | 2.20 | 629.6 | 687.7 | 1 |
| 122 | 2.67 | 638.7 | 696.9 | 1 |
| 123 | 2.50 | 663.8 | 721.7 | 1 |
| 38 | 2.41 | 617.8 | 675.8 | 1 |
| 39 | 2.49 | 585.7 | 643.7 | 1 |
| 40 | 2.32 | 641.8 | 699.9 | 1 |
| 42 | 2.34 | 641.7 | 699.6 | 1 |
| 43 | 2.46 | 641.7 | 699.7 | 1 |
| 41 | 2.37 | 641.7 | 699.7 | 1 |
| 44 | 2.55 | 691.8 | 749.7 | 1 |
| 137 | 2.31 | 628.7 | 686.6 | 1 |
| 45 | 2.29 | 641.7 | 699.8 | 1 |
| 46 | 2.30 | 641.7 | 699.8 | 1 |
| 72 | 2.34 | 626.7 | 684.6 | 1 |
| 73 | 2.47 | 626.7 | 684.6 | 1 |
| 174 | 2.31 | 682.8 | 740.7 | 1 |
| 175 | 2.38 | 682.7 | 740.7 | 1 |
| 47 | 2.24 | 629.8 | 688.2 | 1 |
| 29 | 2.51 | 629.6 | 687.7 | 1 |
| 124 | 2.49 | 643.8 | 701.8 | 1 |
| 125 | 2.21 | 601.7 | 659.8 | 1 |
| 126 | 2.30 | 601.7 | 659.7 | 1 |
| 127 | 3.09 | 649.8 | 707.9 | 1 |
| 128 | 3.19 | 649.8 | 707.7 | 1 |
| 129 | 2.27 | 643.8 | 701.9 | 1 |
| 130 | 2.37 | 643.8 | 701.7 | 1 |
| 88 | 2.19 | 629.8 | 687.8 | 1 |
| 48 | 2.44 | 613.8 | 671.8 | 1 |
| 49 | 2.57 | 613.7 | 671.8 | 1 |
| 172 | 2.27 | 668.8 | 726.8 | 1 |
| 170 | 2.25 | 668.7 | 726.7 | 1 |
| 173 | 2.35 | 668.8 | 726.6 | 1 |
| 171 | 2.35 | 668.8 | 726.8 | 1 |
| 50 | 2.39 | 684.8 | 742.9 | 1 |
| 51 | 2.34 | 684.8 | 742.8 | 1 |
| 52 | 2.47 | 684.8 | 742.8 | 1 |
| 140 | 2.50 | 651.7 | 709.8 | 1 |
| 142 | 2.57 | 665.7 | 723.7 | 1 |
| 143 | 2.67 | 665.7 | 723.7 | 1 |
| 97 | 2.37 | 641.7 | 699.7 | 1 |
| 144 | 2.44 | 680.7 | 738.7 | 1 |
| 80 | 2.20 | 656.7 | 714.7 | 1 |
| 74 | 2.29 | 572.4 | — | 16 |
| 75 | 2.42 | 572.4 | — | 16 |
| 76 | 2.34 | 683.4 | — | 16 |
| 78 | 2.34 | 628.4 | 686.5 | 16 |
| 98 | 2.35 | 691.7 | 749.9 | 1 |
| 99 | 2.19 | 629.7 | 687.8 | 1 |
| 100 | 2.31 | 684.8 | 742.8 | 1 |
| 102 | 2.22 | 627.6 | 685.7 | 1 |
| 103 | 2.34 | 627.7 | 685.6 | 1 |
| 53 | 2.27 | 682.8 | 740.8 | 1 |
| 54 | 2.37 | 682.8 | 740.8 | 1 |
| 55 | 2.35 | 682.8 | 740.8 | 1 |
| 56 | 2.40 | 682.7 | 740.7 | 1 |
| 57 | 2.36 | 682.7 | 740.7 | 1 |
| 58 | 2.39 | 682.7 | 740.8 | 1 |
| 59 | 2.31 | 682.8 | 740.8 | 1 |
| 60 | 2.30 | 682.8 | 740.9 | 1 |
| 61 | 2.35 | 670.8 | 728.8 | 1 |
| 81 | 2.25 | 629.7 | 687.8 | 1 |
| 141 | 2.61 | 651.7 | 709.8 | 1 |
| 82 | 2.17 | 627.7 | 685.8 | 1 |
| 83 | 2.29 | 627.7 | 685.7 | 1 |
| 84 | 2.24 | 627.7 | 685.8 | 1 |
| 84a | 2.33 | 627.7 | 685.8 | 1 |
| 101 | 2.32 | 627.7 | 685.8 | 1 |
| 104 | 2.18 | 656.7 | 714.8 | 1 |
| 105 | 2.26 | 656.7 | 714.7 | 1 |
| 89 | 2.40 | 655.4 | 713.6 | 16 |
| 62 | 2.37 | 698.8 | 756.7 | 1 |
| 63 | 2.49 | 698.8 | 756.8 | 1 |
| 64 | 2.28 | 682.8 | 740.8 | 1 |
| 65 | 2.39 | 682.8 | 740.8 | 1 |
| 95 | 2.66 | 651.8 | 709.8 | 1 |
| 96 | 2.48 | 680.7 | 738.7 | 1 |
| 66 | 2.32 | 682.7 | 740.7 | 1 |
| 67 | 2.32 | 682.7 | 740.7 | 1 |
| 107 | 2.29 | 627.6 | 685.6 | 1 |
| 91 | 2.42 | 572.4 | — | 16 |
| 92 | 2.34 | 683.4 | — | 16 |
| 93 | 2.52 | 628.8 | 686.8 | 1 |
| 79 | 2.49 | 680.8 | 738.9 | 1 |
| 90 | 2.29 | 572.4 | — | 16 |
| 94 | 2.52 | 651.6 | 709.6 | 1 |
| 199 | 1.13 | 626.5 | 684.8 | 18 |
| 205 | 2.62 | 689.4 | 747.5 | 16 |
| 204 | 2.41 | 689.3 | 747.5 | 16 |
| 231 | 3.404 | 655.3 | | 19 |
| 232 | 4.633 | 655.3 | | 4 |
| 233 | 3.065 | 641.3 | | 19 |
| 234 | 2.99 | 627.4 | | 19 |
| 235 | 4.414 | 649.2 | | 4 |
| 236 | 3.063 | 659.3 | | 19 |
| 237 | 3.062 | 659.3 | | 19 |
| 238 | 3.007 | 617.3 | | 19 |
| 239 | 3.003 | 617.3 | | 19 |
| 240 | 4.472 | 628.4 | | 4 |
| 241 | 4.291 | 628.3 | | 4 |
| 242 | 4.551 | 679.2 | | 4 |
| 243 | 4.394 | 656.1 | | 4 |
| 244 | 3.047 | 641.3 | | 19 |
| 245 | 3.07 | 641.2 | | 19 |
| 246 | 5.156 | 641.5 | | 4 |
| 247 | 2.274 | 641.3 | | 19 |
| 248 | 4.691 | 682.2 | | 4 |
| 249 | 4.486 | 703.2 | | 4 |
| 250 | 2.994 | 613.3 | | 19 |
| 251 | 2.816 | 655.3 | | 19 |
| 252 | 2.909 | 655.3 | | 19 |
| 253 | 2.915 | 655.3 | | 19 |
| 254 | 2.873 | 641.3 | | 19 |
| 255 | 2.882 | 641.4 | | 19 |
| 256 | 2.894 | 643.3 | | 19 |
| 257 | 2.931 | 629.3 | | 19 |
| 258 | 2.828 | 641.3 | | 19 |
| 259 | 4.726 | 641.3 | | 4 |
| 260 | 2.911 | 615.4 | | 19 |
| 261a | 2.830 | 573.3 | | 19 |
| 261b | 2.827 | 573.3 | | 19 |
| 262 | 4.55 | 617.3 | | 4 |
| 263 | 3.268 | 629.4 | | 19 |
| 264 | 3.285 | 629.4 | | 19 |
| 265 | 3.317 | 641.4 | | 19 |
| 266 | 4.869 | 655.3 | | 4 |
| 267 | 4.969 | 655.3 | | 4 |
| 268 | 4.489 | 641.3 | | 4 |
| 269 | 4.618 | 641.3 | | 4 |
| 270 | 2.987 | 655.4 | | 19 |
| 271 | 5.388 | 655.3 | | 4 |
| 272 | 2.102 | 585.3 | | 19 |
| 273 | 2.118 | 585.1 | | 19 |
| 274 | 3.052 | 641.3 | | 19 |
| 275 | 4.69 | 643.2 | | 4 |
| 276 | 4.878 | 643.2 | | 4 |
| 277 | 4.061 | 656.2 | | 4 |
| 278 | 4.685 | 641.3 | | 4 |
| 279 | 4.856 | 641.4 | | 4 |
| 280a | 2.998 | 655.2 | | 19 |
| 280b | 3.005 | 655.2 | | 19 |
| 281a | 4.476 | 655.6 | | 21 |
| 281b | 4.623 | 655.6 | | 21 |
| 282a | 3.012 | 641.2 | | 19 |
| 282b | 3.006 | 641.3 | | 19 |
| 283a | 4.662 | 585.2 | | 4 |
| 283b | 4.774 | 585.2 | | 4 |
| 284a | 5.065 | 585.4 | | 21 |
| 284b | 4.733 | 585.3 | | 21 |
| 285 | 3.101 | 676.4 | | 19 |
| 286 | 2.955 | 676.3 | | 19 |
| 287 | 2.221 | 676.2 | | 19 |
| 288 | 3.011 | 626.3 | | 19 |
| 289 | 3.005 | 640.2 | | 19 |
| 290 | 2.992 | 626.3 | | 19 |
| 291 | 2.993 | 626.3 | | 19 |
| 1a | 3.134 | 545.2 | | 19 |
| 1b | 3.145 | 545.2 | | 19 |
| 2b | 3.349 | 587.4 | | 19 |
| 296 | 4.413 | 643.3 | | 4 |
| 297 | 0.815 | 644.4 | | 24 |
| 298 | 3.48 | 612.2 | | 19 |
| 299 | 3.488 | 617.3 | | 19 |
| 300 | 3.725 | 629.3 | | 19 |
| 301 | 4.787 | 631.2 | | 4 |
| 302 | 3.169 | 644.3 | | 4 |
| 303 | 3.488 | 613.7 | | 19 |
| 304 | 3.185 | 613.3 | | 19 |
| 305 | 3.352 | 631.3 | | 19 |
| 306 | 3.235 | 588.3 | | 19 |
| 307 | 4.162 | 602.3 | | 4 |
| 308 | 3.135 | 602.3 | | 19 |
| 309 | 3.247 | 629.2 | | 19 |
| 310 | 3.177 | 629.3 | | 19 |
| 311 | 3.655 | 647.3 | | 19 |
| 312 | 4.348 | 586.3 | | 4 |
| 313 | 4.264 | 599.3 | | 4 |
| 314 | 4.304 | 599.3 | | 4 |
| 315 | 4.714 | 668.4 | | 4 |
| 316 | 4.281 | 668.1 | | 4 |
| 317 | 3.118 | 656.4 | | 19 |
| 318 | 2.251 | 656.2 | | 19 |
| 319 | 3.283 | 601.2 | | 19 |
| 320 | 3.274 | 601.2 | | 19 |
| 321 | 4.702 | 600.3 | | 4 |
| 322 | 4.702 | 600.3 | | 4 |
| 323 | 4.146 | 601.2 | | 4 |
| 324 | 3.059 | 601.3 | | 19 |
| 325 | 3.042 | 641.3 | | 19 |
| 326 | 3.055 | 641.3 | | 19 |
| 327 | 3.116 | 655.4 | | 19 |
| 328 | 4.638 | 670.3 | | 4 |
| 329 | 3.077 | 684.1 | | 19 |
| 330 | 4.692 | 643.3 | | 4 |
| 331 | 2.919 | 629.4 | | 19 |
| 332 | 2.869 | 629.3 | | 19 |
| 333 | 3.323 | 654.3 | | 19 |
| 335 | 3.319 | 653.3 | | 19 |
| 336 | 3.318 | 653.3 | | 19 |
| 339 | 2.148 | 669.1 | | 19 |
| 340 | 2.919 | 653.3 | | 4 |
| 341 | 4.52 | 715 | | 4 |
| 342 | 3.411 | 611.3 | | 19 |
| 343 | 4.806 | 612.3 | | 4 |
| 344 | 3.483 | 627.4 | | 19 |
| 345 | 3.011 | 585.3 | | 19 |
| 346 | 2.994 | 585.2 | | 19 |
| 348 | 3.199 | 658.5 | | 19 |
| 350 | 4.342 | 644.3 | | 4 |
| 351 | 1.872 | 656.3 | | 20 |
| 353 | 3.564 | 601.6 | | 22 |
| 354 | 3.564 | 601.9 | | 22 |
| 356 | 1.821 | 601.4 | | 25 |
| 357 | 3.156 | 643.3 | | 19 |
| 358 | 3.083 | 643.4 | | 19 |
| 371 | 0.423 | 681.30 | | 26 |
| 372 | 0.423 | 681.35 | | 26 |
| 373 | 0.699 | 681.3 | | 27 |
| 374 | 0.698 | 681.3 | | 28 |
| 377 | 2.685 | 696.4 | | 29 |
| 378 | 3.196 | 696.4 | | 19 |
| 385 | 2.225 | 708.3 | | 20 |
| 390 | 3.162 | 682.3 | | 19 |
| 391 | 3.352 | 726.3 | | 19 |
| 392 | 3.275 | 708.3 | | 19 |
| 393 | 4.740 | 670.3 | | 21 |
| 375 | 3.147 | 696.4 | | 19 |
| 394 | 4.515 | 682.3 | | 21 |
| 379 | 1.630 | 655.3 | | 25 |
| 380 | 1.669 | 722.3 | | 25 |
| 381 | 1.651 | 696.3 | | 25 |
| 382 | 1.712 | 655.3 | | 25 |
| 383 | 1.671 | 684.3 | | 25 |
| 384 | 1.656 | 696.3 | | 25 |
| 386 | 1.664 | 696.3 | | 25 |
| 387 | 1.656 | 696.3 | | 25 |
| 388 | 1.607 | 670.2 | | 25 |
| 389 | 1.591 | 670.3 | | 25 |
| 395 | 2.42 | 670.8 | 728.8 | 1 |
| 396 | 2.58 | 655.7 | 713.8 | 1 |
| 397 | 2.44 | 641.8 | 699.8 | 1 |
| 376 | 4.846 | 696.3 | | 4 |
| 398 | 1.458 | 643.30 | | 30 |
| 399 | 1.538 | 643.30 | | 30 |
| 402 | 1.637 | 655.55 | | 31 |
| 403 | 1.732 | 655.40 | | 31 |
| 400 | 1.470 | 696.45 | | 31 |
| 401 | 1.547 | 696.50 | | 31 |
| 405 | 4.484 | 680.3 | | 4 |
| 406 | 1.236 | 661.4 | | 32 |
| 407 | 4.731 | 679.3 | | 4 |
| 408 | 3.523 | 716.4 | | 19 |
| 409 | 4.985 | 679.3 | | 4 |
| 410 | 3.211 | 679.4 | | 19 |
| 411 | 3.354 | 663.3 | | 19 |
| 412 | 4.610 | 662.3 | | 4 |
| 413 | 3.179 | 663.3 | | 19 |
| 414 | 3.152 | 680.3 | | 19 |
| 415 | 3.296 | 715.3 | | 19 |
| 416 | 3.408 | 683.3 | | 19 |
| 417 | 3.432 | 699.3 | | 19 |
| 418 | 3.212 | 680.3 | | 19 |
| 419 | 3.205 | 653.4 | | 19 |
| 420 | 3.194 | 689.3 | | 19 |
| 421 | 3.268 | 694.3 | | 19 |
| 422 | 4.806 | 662.3 | | 4 |
| 423 | 4.652 | 703.3 | | 4 |
| 424 | 3.471 | 689.3 | | 19 |
| 425 | 3.220 | 679.3 | | 19 |
| 426 | 3.301 | 675.3 | | 19 |
| 427 | 3.333 | 695.3 | | 19 |
| 428 | 3.277 | 721.3 | | 19 |
| 429 | 3.193 | 697.3 | | 19 |
| 430 | 4.586 | 703.6 | | 4 |
| 431 | 3.405 | 663.3 | | 19 |
| 432 | 3.272 | 680.3 | | 19 |
| 433 | 4.672 | 662.5 | | 4 |
| 434 | 0.80 | 638.4 | | 39 |
| 435 | 1.72 | 683.5 | | 38 |
| 436 | 0.82 | 669.4 | | 1 |
| 437 | 0.86 | 685.3 | | 33 |
| 438 | 1.73 | 669 | | 34 |
| 439 | 0.82 | 670 | | 33 |
| 440 | 0.76 | 517.4 | | 35 |
| 441 | 0.77 | 517.4 | | 35 |
| 442 | 1.85 | 686 | | 36 |
| 443 | 1.75 | 699 | | 34 |
| 444 | 0.77 | 585.3 | | 33 |
| 445 | 0.96 | 710.3 | | 33 |
| 446 | 0.82 | 671.3 | | 33 |
| 447 | 1.82 | 644.2 | | 37 |
| 448 | 2.29 | 662.4 | | 37 |
| 449 | 2.10 | 685.2 | | 37 |
| 450 | 1.94 | 679 | | 34 |
| 451 | 1.85 | 679 | | 34 |
| 452 | 1.79 | 680 | | 34 |
| 453 | 1.08 | 685.5 | | 35 |
| 454 | 2.64 | 686.2 | | 37 |
| 455 | 0.86 | 657.5 | | 35 |
| 456 | 0.97 | 603.5 | | 35 |
| 457 | 1.00 | 680.5 | | 35 |
| 458 | 2.16 | 663.2 | | 37 |
| 459 | 1.98 | 683.5 | | 36 |
| 460 | 1.84 | 683.5 | | 36 |
| 461 | 1.64 | 641 | | 36 |
| 462 | 1.75 | 677.2 | | 37 |
| 502 | 3.48 | 576 | | 40 |
| 503 | 1.78 | 576 | | 33 |
| 504 | 1.19 | 648 | | 33 |
| 505 | 0.94 | 648 | | 33 |
| 506 | 1.31 | 682 | | 33 |
| 507 | 1.05 | 682 | | 33 |
| 508 | 2.5 | 658.4 | 717 | 40 |
| 510 | 0.85 | 693.1 | | 41 |
| 511 | 0.8 | 659.2 | | 41 |
| 512a | 1.88 | 617 | | 40 |
| 512b | 1.87 | 617 | | 40 |
| 463 | 0.63 | 638.3 | | 41 |
| 464 | 0.70 | 559.2 | | 41 |
| 465 | 2.13 | 698.4 | | 23 |
| 466 | 1.25 | 670.3 | | 23 |
| 467 | 2.17 | 700.3 | | 23 |
| 468 | 1.04 | 686.4 | | 45 |
| 470 | 3.49 | 762.6 | | 29 |
| 471 | 0.7 | 658.4 | | 41 |
| 472 | 0.65 | 656.3 | | 41 |
| 473 | 0.71 | 647.4 | | 41 |
| 474 | 0.26 | 793.1 | | 41 |
| 475 | 1.85 | 681.3 | | 23 |
| 476 | 0.89 | 738.5 | | 24 |
| 477 | 0.79 | 638.5 | | 24 |
| 478 | 2.09 | 684 | | 36 |
| 479 | 1.38 | 656.5 | | 38 |
| 480 | 2.08 | 684 | | 36 |
| 481 | 2.05 | 685.3 | | 40 |
| 482 | 2.1 | 701 | | 36 |
| 483 | 2.21 | 714 | | 40 |
| 485 | 2.14 | 680 | | 36 |
| 486 | 1.97 | 680 | | 36 |
| 487 | 2.05 | 695 | | 40 |
| 488 | 1.8 | 644.3 | | 37 |
| 489 | 0.9 | 681.4 | | 33 |
| 491a | 0.82 | 645.3 | | 41 |
| 491b | 0.85 | 645.4 | | 41 |
| 492 | 0.70 | 646.4 | | 41 |
| 493 | 0.70 | 634.4 | | 41 |
| 494 | 1.57 | 671.4 | 729.6 | 42 |
| 495 | 0.72 | 683.4 | | 41 |
| 496 | 1.53 | 671.5 | | 18 |
| 497 | 1.26 | 726.5 | | 42 |
| 498 | 1.32 | 726.5 | | 42 |
| 499 | 1.13 | 575.3 | | 43 |
| 500 | 0.766 | 617.55 | | 44 |
| 501 | 1.337 | 675.30 | | 43 |

### Pharmacological Part

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris.HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2× FITC-MBM1 peptide (FITC-β-alanine-SARWRFPARPGT-NH₂ (SEQ ID NO: 3)) (“FITC” means fluorescein isothiocyanate) in assay buffer was added, the microtiter plate centrifuged at 1000 rpm for 1 min and the assay mixtures incubated for 15 min at ambient temperature. The relative amount of menin FITC-MBM1 complex present in an assay mixture is determined by measuring the homogenous time-resolved fluorescence (HTRF) of the terbium/FITC donor/acceptor fluorophore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

% inhibition=((HC−LC)−(HTRF^{compound}−LC))/(HC−LC))*100 (Eqn 1)

Where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

% inhibition=Bottom+(Top−Bottom)/(1+10\hat{ }((logIC₅₀−log[cmpd])*h)) (Eqn 2)

Where Bottom and Top are the lower and upper asymptotes of the dose-response curve, respectively, IC₅₀ is the concentration of compound that yields 50% inhibition of signal and h is the Hill coefficient. IC₅₀ values below 0.1 nM in the HTRF assay were reported as 0.1 nM in the Table below (detection limit).

Preparation of Terbium cryptate labeling of Menin: Menin (a.a 1-610-6xhis tag (“6xhis tag” disclosed as SEQ ID NO: 2), 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1× Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1 M Hepes, pH 7+0.1% BSA (bovine serum albumin)). The other half was eluted with 0.1 M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLAV
NRVIPTNVPELTFQPSPAPDPPGGLTYFPVADLSIIAALYARFTAQIRGA
VDLSLYPREGGVSSRELVKKVSDVIWNSLSRSYFKDRAHIQSLFSFITGT
KLDSSGVAFAVVGACQALGLRDVHLALSEDHAWVVFGPNGEQTAEVTWHG
KGNEDRRGQTVNAGVAERSWLYLKGSYMRCDRKMEVAFMVCAINPSIDLH
TDSLELLQLQQKLLWLLYDLGHLERYPMALGNLADLEELEPTPGRPDPLT
LYHKGIASAKTYYRDEHIYPYMYLAGYHCRNRNVREALQAWADTATVIQD
YNYCREDEEIYKEFFEVANDVIPNLLKEAASLLEAGEERPGEQSQGTQSQ
GSALQDPECFAHLLRFYDGICKWEEGSPTPVLHVGWATFLVQSLGRFEGQ
VRQKVRIVSREAEAAEAEEPWGEEAREGRRRGPRRESKPEEPPPPKKPAL
DKGLGTGQGAVSGPPRKPPGTVAGTARGPEGGSTAQVPAPAASPPPEGPV
LTFQSEKMKGMKELLVATKINSSAIKLQLTAQSQVQMKKQKVSTPSDYTL
SFLKRQRKGLHHHHHH

### 2) Proliferation Assay

The anti-proliferative effect of menin/MLL protein/protein interaction inhibitor test compounds was assessed in human leukemia cell lines. The cell line MOLM14 harbors a MLL translocation and expresses the MLL fusion proteins MLL-AF9, respectively, as well as the wildtype protein from the second allele. MLL rearranged cell lines (e.g. MOLM14) exhibit stem cell-like HOXA/MEIS1 gene expression signatures. KO-52 was used as a control cell line containing two MLL (KMT2A) wildtype alleles in order to exclude compounds that display general cytotoxic effects.

MOLM14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 cell lines were propagated in alpha-MEM (Sigma Aldrich) supplemented with 20% heat-inactivated fetal bovine serum (HyClone), 2 mM L-glutamine (Sigma Aldrich) and 50 μg/ml gentamycin (Gibco). Cells were kept at 0.3-2.5 million cells per ml during culturing and passage numbers did not exceed 20.

In order to assess the anti-proliferative effects, 200 MOLM14 cells, or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:

LC=Low Control: cells treated with e.g. 1 μM of the cytotoxic agent staurosporin, or e.g. cells treated with a high concentration of an alternative reference compound

$$HC = High Control \text{:} Mean confluence ( \% ) ( DMSO treated cells )$$

$$\% Effect = 100 - ( 100 * ( Sample - LC ) / ( HC - LC ) )$$

GraphPad Prism (version 7.00) was used to calculate the IC₅₀. Dose-response equation was used for the plot of % Effect vs Log 10 compound concentration with a variable slope and fixing the maximum to 100% and the minimum to 0%.

TABLE 3
Biological data

| | | spheroid | spheroid |
|---|---|---|---|
| | HTRF-30 min | assay_OneTime | assay_OneTime |
| | incubation | MOLM14 | KO-52 |
| Co. No. | IC50(nM) | IC50(μM) | IC50(μM) |
| 2 | 0.4 | 0.029 | 9.04 |
| 4 | 0.3 | 0.066 | 7.73 |
| 5 | 0.2 | 0.120 | 9.31 |
| 6 | 0.3 | 0.280 | 6.47 |
| 7 | 0.1 | 0.004 | 5.04 |
| 8 | 0.3 | 0.052 | 10.21 |
| 9 | 0.1 | 0.021 | 7.16 |
| 10 | 0.4 | 0.017 | 5.09 |
| 12 | | 0.002 | >15 |
| 14 | 0.1 | 0.002 | 2.11 |
| 16 | 0.1 | 0.004 | 1.88 |
| 18 | 0.1 | 0.002 | 0.81 |
| 20 | 0.1 | 0.005 | 4.30 |
| 21 | 0.1 | 0.052 | 4.61 |
| 22 | 0.1 | 0.003 | 10.77 |
| 23 | 0.3 | 0.009 | 1.06 |
| 25 | 0.1 | 0.085 | 5.57 |
| 26 | 6.2 | >0.94 | >15 |
| 27 | 0.1 | 0.036 | 5.56 |
| 29 | 1.8 | 0.220 | >15 |
| 30 | 0.3 | 0.029 | 2.58 |
| 31 | 0.3 | 0.007 | 2.41 |
| 33 | 0.3 | 0.007 | 2.49 |
| 34 | 0.5 | 0.036 | 5.14 |
| 35 | 1.3 | 0.340 | >15 |
| 36 | 0.2 | 0.010 | 3.05 |
| 38 | 0.2 | 0.006 | >15 |
| 39 | 0.1 | 0.007 | 1.48 |
| 40 | 0.2 | 0.010 | 0.50 |
| 41 | 0.2 | 0.012 | >15 |
| 42 | 0.1 | 0.004 | 2.12 |
| 43 | 0.1 | 0.042 | 9.96 |
| 44 | 0.1 | 0.027 | |
| 45 | 0.2 | <0.002 | 6.33 |
| 46 | 0.1 | 0.002 | 5.93 |
| 47 | 0.2 | 0.003 | 4.36 |
| 48 | 0.3 | 0.004 | 1.06 |
| 49 | 0.6 | 0.027 | 2.13 |
| 50 | 0.1 | <0.002 | >15 |
| 51 | 0.1 | 0.014 | 14.44 |
| 52 | 0.4 | 0.210 | 14.03 |
| 53 | 0.2 | 0.005 | 9.28 |
| 54 | 0.3 | 0.028 | 4.96 |
| 55 | 0.5 | 0.095 | 6.43 |
| 56 | 0.4 | 0.350 | >15 |
| 57 | 0.1 | 0.008 | 7.54 |
| 58 | 0.1 | 0.019 | >15 |
| 59 | 0.1 | 0.024 | 5.01 |
| 60 | 0.2 | 0.007 | 3.37 |
| 61 | 0.2 | 0.023 | |
| 62 | 0.1 | 0.004 | 6.26 |
| 63 | | 0.057 | >15 |
| 64 | 0.1 | 0.002 | >15 |
| 65 | 0.2 | 0.130 | 11.83 |
| 66 | 0.1 | 0.006 | >15 |
| 67 | 0.1 | 0.007 | >15 |
| 68 | 0.1 | 0.005 | 4.41 |
| 69 | 0.1 | 0.039 | 14.03 |
| 70 | 0.1 | 0.004 | 5.15 |
| 71 | 0.1 | 0.060 | 9.73 |
| 72 | 0.1 | 0.005 | 1.62 |
| 73 | 0.2 | 0.040 | 7.05 |
| 74 | 1.3 | 0.580 | 2.79 |
| 75 | 0.5 | 0.690 | 4.00 |
| 76 | 8.0 | >0.94 | 5.69 |
| 78 | 2.9 | 0.630 | 4.64 |
| 79 | 0.3 | 0.240 | 3.66 |
| 80 | 43.2 | >0.94 | >15 |
| 81 | 2.0 | 0.280 | >15 |
| 82 | 0.4 | 0.300 | >15 |
| 83 | 1.6 | 0.980 | >15 |
| 84 | 5.5 | 0.220 | >15 |
| 85 | 25.4 | 1.560 | >15 |
| 86 | 17.2 | 0.650 | >15 |
| 87 | 1.9 | 0.820 | >15 |
| 88 | 2.3 | 0.410 | >15 |
| 89 | 0.2 | 0.020 | 6.18 |
| 90 | 0.1 | 0.002 | 0.54 |
| 91 | 0.1 | 0.026 | 1.03 |
| 92 | 0.1 | 0.007 | 0.78 |
| 93 | 0.1 | 0.003 | 0.49 |
| 94 | 0.1 | 0.025 | 3.39 |
| 95 | 0.1 | 0.210 | 11.35 |
| 96 | 153.1 | >0.94 | >15 |
| 97 | 0.1 | 0.003 | 0.31 |
| 98 | 0.4 | 0.058 | 4.35 |
| 99 | 0.1 | 0.002 | 2.00 |
| 100 | 0.1 | 0.033 | 9.81 |
| 101 | 0.1 | 0.010 | 1.07 |
| 102 | 0.1 | 0.012 | 8.47 |
| 103 | 0.1 | 0.100 | 12.21 |
| 104 | 0.2 | 0.073 | 11.80 |
| 105 | 1.0 | | |
| 106 | 0.1 | 0.011 | 4.99 |
| 107 | 0.1 | 0.076 | >15 |
| 108 | 0.2 | 0.001 | 2.65 |
| 109 | 0.1 | 0.053 | >15 |
| 110 | 0.1 | 0.012 | 6.84 |
| 111 | 0.2 | 0.120 | >15 |
| 112 | 0.1 | 0.006 | 10.14 |
| 113 | 0.2 | 0.200 | >15 |
| 114 | 0.1 | 0.008 | 14.22 |
| 115 | 0.2 | 0.430 | >15 |
| 116 | 0.1 | 0.007 | 8.89 |
| 117 | 0.1 | 0.170 | >15 |
| 120 | 0.3 | 0.073 | >15 |
| 121 | 0.1 | 0.007 | 2.78 |
| 122 | 0.2 | 0.055 | >15 |
| 123 | 5.4 | 1.910 | >15 |
| 124 | 0.2 | 0.012 | 2.52 |
| 125 | 0.1 | 0.012 | 5.30 |
| 126 | 0.4 | 0.150 | 14.90 |
| 127 | 0.5 | 0.160 | 4.66 |
| 128 | 1.3 | 0.450 | 7.89 |
| 129 | 0.2 | 0.011 | 3.39 |
| 130 | | 0.120 | >15 |
| 131 | 0.2 | 0.050 | 6.20 |
| 132 | 0.3 | 0.076 | 8.10 |
| 133 | 0.1 | 0.021 | 7.38 |
| 134 | | 0.130 | 13.20 |
| 135 | 0.3 | 0.008 | 1.00 |
| 136 | 0.1 | 0.008 | 7.07 |
| 137 | 0.2 | 0.007 | |
| 138 | 0.1 | 0.002 | 0.46 |
| 139 | 0.1 | 0.041 | 1.31 |
| 140 | 0.5 | 0.042 | |
| 141 | | 0.260 | 12.37 |
| 142 | 0.1 | 0.007 | 1.35 |
| 143 | 0.1 | 0.027 | 1.53 |
| 144 | 0.8 | 0.200 | 12.31 |
| 145 | 0.2 | 0.006 | 3.58 |
| 146 | 0.2 | 0.160 | 6.60 |
| 147 | 0.1 | 0.004 | 0.39 |
| 148 | 0.2 | 0.003 | 0.61 |
| 149 | 0.1 | <0.002 | 0.51 |
| 150 | 0.1 | 0.120 | 3.14 |
| 151 | 0.1 | 0.004 | 2.03 |
| 152 | 0.2 | 0.049 | 3.96 |
| 153 | 5.3 | 0.730 | 4.83 |
| 154 | 0.4 | 0.230 | 9.08 |
| 155 | 2.3 | 0.570 | >15 |
| 156 | 0.8 | 0.100 | 7.96 |
| 157 | 0.2 | 0.120 | 7.77 |
| 158 | 0.8 | 0.480 | >15 |
| 159 | 0.3 | 0.110 | 1.10 |
| 160 | 23.2 | >3.75 | >15 |
| 161 | 0.1 | 0.004 | 3.36 |
| 162 | 0.1 | 0.110 | 6.40 |
| 163 | 0.1 | 0.011 | 4.37 |
| 164 | 0.1 | 0.010 | 1.41 |
| 165 | 0.8 | 0.200 | 9.91 |
| 166 | | 0.003 | 1.53 |
| 167 | 0.1 | <0.002 | 4.11 |
| 168 | 0.6 | 0.053 | 2.59 |
| 169 | 0.5 | 0.051 | |
| 170 | 0.1 | 0.005 | 8.68 |
| 171 | 0.1 | 0.031 | >15 |
| 172 | 0.1 | 0.022 | 10.09 |
| 173 | 0.2 | 0.150 | >15 |
| 174 | 0.1 | 0.009 | 7.40 |
| 175 | 0.1 | 0.110 | >15 |
| 176 | 0.5 | 0.080 | 10.43 |
| 177 | 0.2 | 0.037 | 8.87 |
| 178 | 108.4 | >0.94 | >15 |
| 179 | 0.4 | 0.073 | 12.61 |
| 180 | 0.3 | 0.062 | 10.14 |
| 181 | 96.9 | >0.94 | >15 |
| 182 | 0.6 | | |
| 183 | 0.4 | 0.160 | |
| 184 | 0.2 | 0.010 | 0.60 |
| 185 | 0.1 | 0.006 | 6.04 |
| 186 | 5.4 | >0.94 | 7.05 |
| 187 | 0.8 | 0.065 | >15 |
| 188 | 0.2 | 0.520 | >15 |
| 189 | 0.4 | 0.140 | >15 |
| 190 | | 0.076 | 14.79 |
| 191 | | >0.94 | >15 |
| 192 | 4.8 | 1.040 | |
| 193 | 0.2 | 0.050 | >15 |
| 194 | 0.9 | 0.450 | >15 |
| 195 | 1.3 | 0.510 | >15 |
| 196 | 13.6 | >0.94 | >15 |
| 198 | 0.3 | 0.250 | >15 |
| 200 | 0.1 | 0.016 | 9.71 |
| 201 | 0.2 | 0.006 | 4.24 |
| 202 | 0.7 | | |
| 203 | 0.8 | 0.300 | 1.86 |
| 204 | 0.3 | 0.190 | >15 |
| 205 | 0.1 | 0.039 | 8.47 |
| 206 | 0.2 | 0.009 | 7.95 |
| 207 | 0.2 | 0.033 | 3.23 |
| 208 | 0.3 | 0.029 | 9.13 |
| 209 | 0.3 | 0.016 | 5.26 |
| 210 | 0.1 | 0.005 | |
| 211 | 0.1 | 0.003 | >15 |
| 212 | 0.2 | 0.008 | |
| 213 | 0.1 | 0.041 | 3.91 |
| 214 | 0.1 | <0.002 | 3.63 |
| 215 | 0.1 | 0.002 | 7.94 |
| 231 | 0.1 | 0.007 | 5.52 |
| 232 | 0.1 | 0.007 | 1.80 |
| 233 | 0.1 | 0.002 | 0.84 |
| 234 | 0.1 | 0.009 | 6.28 |
| 235 | 0.5 | 0.003 | |
| 236 | 0.2 | 0.004 | 2.25 |
| 237 | 0.2 | 0.036 | 7.90 |
| 238 | 0.1 | 0.002 | 7.17 |
| 239 | 0.3 | 0.016 | 2.81 |
| 240 | 1.9 | 0.270 | |
| 241 | 2.1 | 0.510 | |
| 242 | 0.1 | 0.008 | 12.15 |
| 243 | 2.5 | 0.580 | >15 |
| 244 | 1.6 | 0.130 | |
| 245 | 0.6 | 0.460 | 8.94 |
| 246 | 0.3 | 0.180 | |
| 247 | 0.1 | 0.009 | |
| 248 | 0.1 | 0.004 | 1.46 |
| 249 | 0.1 | 0.009 | |
| 250 | 0.1 | 0.008 | 0.94 |
| 251 | 0.2 | 0.007 | 1.24 |
| 252 | 12.7 | 0.270 | >15 |
| 253 | 0.1 | 0.010 | 1.62 |
| 254 | 0.1 | 0.016 | >15 |
| 255 | 4.7 | 0.920 | >15 |
| 256 | 0.2 | 0.005 | >15 |
| 257 | 0.1 | 0.006 | >15 |
| 258 | 0.1 | 0.004 | 0.49 |
| 259 | 0.4 | 0.094 | 4.76 |
| 260 | 0.1 | 0.006 | 3.25 |
| 261a | 0.16 | 0.019 | >15 |
| 261b | 0.3 | 0.083 | 3.07 |
| 262 | 0.1 | 0.005 | 1.28 |
| 263 | 0.2 | 0.009 | |
| 264 | 0.8 | 0.170 | 14.39 |
| 265 | 0.1 | 0.011 | |
| 266 | 0.1 | <0.002 | |
| 267 | 1.3 | 0.180 | |
| 268 | 0.1 | 0.006 | |
| 269 | 5.6 | >0.94 | |
| 270 | 0.2 | 0.002 | |
| 271 | 2.4 | 0.260 | >15 |
| 272 | 0.1 | 0.021 | |
| 273 | 0.3 | 0.110 | >15 |
| 274 | 0.1 | 0.002 | 3.56 |
| 275 | 0.1 | 0.007 | |
| 276 | 0.8 | 0.082 | >15 |
| 277 | 0.9 | 0.250 | |
| 278 | 0.1 | 0.002 | |
| 279 | 0.1 | 0.020 | 5.99 |
| 280a | 5.0 | >0.94 | |
| 280b | 2.4 | 0.740 | >15 |
| 281a | 0.17 | 0.010 | |
| 281b | 0.31 | 0.083 | |
| 282a | 2.4 | 0.280 | |
| 282b | 0.5 | 0.380 | 11.48 |
| 283 | 0.5 | 0.200 | 14.40 |
| 284 | 0.3 | 0.320 | 7.33 |
| 285 | 23.3 | >0.94 | >15 |
| 286 | 0.3 | 0.120 | 12.24 |
| 287 | 70.3 | >0.94 | >15 |
| 288 | 0.1 | 0.017 | 12.06 |
| 289 | 0.1 | 0.002 | 0.31 |
| 290 | 0.1 | 0.005 | 1.08 |
| 291 | 0.2 | 0.024 | 1.07 |
| 1a | 0.1 | 0.020 | >15 |
| 1b | 0.4 | 0.200 | >15 |
| 2b | 16.6 | 1.310 | >15 |
| 296 | 0.2 | 0.010 | >15 |
| 297 | 0.1 | 0.010 | 10.60 |
| 298 | 0.1 | 0.006 | 9.32 |
| 299 | 0.2 | 0.034 | 8.35 |
| 300 | 0.3 | 0.170 | 8.19 |
| 301 | 0.2 | 0.033 | 2.50 |
| 302 | 0.1 | 0.021 | 14.14 |
| 303 | 0.3 | 0.007 | 4.53 |
| 304 | 0.1 | 0.032 | 12.13 |
| 305 | 0.2 | 0.017 | 5.78 |
| 306 | 0.3 | 0.056 | 6.46 |
| 307 | 0.7 | >0.94 | >15 |
| 308 | 0.2 | 0.070 | 4.57 |
| 309 | 0.2 | 0.022 | 7.39 |
| 310 | 24.2 | >0.94 | |
| 311 | 0.3 | 0.052 | 9.99 |
| 312 | 0.2 | 0.005 | 1.27 |
| 313 | 43.4 | >0.94 | >15 |
| 314 | 0.2 | 0.120 | 5.87 |
| 315 | 0.1 | 0.018 | 1.96 |
| 316 | 4.9 | | |
| 317 | 0.3 | 0.060 | |
| 318 | 0.1 | 0.026 | 6.90 |
| 319 | 23.7 | >0.94 | >15 |
| 320 | 0.1 | 0.028 | 4.60 |
| 321 | 91.1 | >0.94 | >15 |
| 322 | 0.3 | 0.030 | >15 |
| 323 | 0.2 | 0.053 | |
| 324 | 117.1 | >0.94 | >15 |
| 325 | 0.1 | 0.004 | 4.98 |
| 326 | 0.2 | 0.032 | 9.18 |
| 327 | 0.2 | 0.005 | >15 |
| 328 | 0.1 | 0.033 | |
| 329 | 0.3 | 0.032 | |
| 330 | 0.4 | 0.130 | 5.90 |
| 331 | 0.2 | 0.003 | 2.85 |
| 332 | 0.7 | 0.037 | |
| 333 | 0.1 | 0.038 | 0.82 |
| 335 | 0.2 | 0.030 | 3.24 |
| 336 | 0.6 | 0.180 | 7.65 |
| 339 | 0.4 | >0.94 | >15 |
| 340 | | 0.170 | 11.40 |
| 341 | 3.0 | >0.94 | >15 |
| 342 | 0.3 | 0.082 | 5.84 |
| 343 | 0.2 | 0.015 | 1.14 |
| 344 | 0.1 | 0.054 | 1.59 |
| 345 | 1.0 | 0.055 | 7.40 |
| 346 | 2.3 | 0.700 | >15 |
| 348 | 0.2 | 0.004 | 10.18 |
| 350 | 0.1 | 0.010 | |
| 351 | 0.1 | 0.020 | 1.10 |
| 353 | 0.5 | | |
| 354 | 7.4 | 2.430 | >15 |
| 356 | 0.3 | 0.011 | >15 |
| 357 | 0.1 | 0.045 | 9.79 |
| 358 | 0.1 | 0.023 | 2.21 |
| 2a | 0.2 | 0.022 | 7.00 |
| 84a | 21.5 | 0.850 | >15 |
| 371 | 0.15 | 0.003 | 10.06 |
| 372 | 0.2 | 0.018 | |
| 373 | 0.77 | 0.0076 | 5.41 |
| 374 | 0.19 | 0.14 | |
| 375 | 0.055 | 0.004 | |
| 376 | 0.62 | 0.10 | |
| 377 | 0.137 | 0.0027 | 4.84 |
| 378 | 0.106 | 0.0016 | |
| 379 | 0.11 | 0.0019 | |
| 380 | 0.16 | 0.0037 | |
| 381 | 0.14 | 0.0081 | |
| 382 | 0.08 | 0.007 | |
| 383 | 0.04 | 0.0023 | |
| 384 | 0.085 | 0.0007 | |
| 385 | 0.026 | <0.0018 | |
| 386 | 0.059 | <0.0018 | |
| 387 | 0.026 | <0.0018 | |
| 388 | 0.027 | <0.0018 | |
| 389 | 0.046 | 0.002 | |
| 390 | 0.18 | 0.004 | |
| 391 | 0.057 | <0.0018 | |
| 392 | 0.11 | 0.002 | |
| 393 | 0.13 | <0.0018 | |
| 394 | 0.083 | 0.003 | |
| 395 | 0.071 | 0.007 | |
| 396 | 0.059 | <0.0018 | |
| 397 | 1.31 | 0.044 | |
| 398 | 0.144 | 0.0064 | |
| 399 | 0.92 | 0.20 | |
| 400 | 0.26 | 0.021 | |
| 401 | 0.52 | 0.26 | |
| 402 | 0.17 | 0.009 | |
| 403 | 0.59 | 0.23 | |
| 405 | 0.22 | 0.003 | 7.57 |
| 406 | 0.30 | 0.042 | |
| 407 | 0.11 | <0.0018 | |
| 408 | 0.14 | 0.22 | |
| 409 | 0.14 | 0.008 | |
| 410 | 0.11 | 0.019 | |
| 411 | 0.15 | 0.01 | |
| 412 | 0.16 | <0.0018 | |
| 413 | 0.08 | 0.026 | |
| 414 | 0.10 | 0.021 | |
| 415 | 0.13 | 0.13 | |
| 416 | 0.17 | 0.0089 | |
| 417 | 0.05 | 0.0018 | |
| 418 | 0.07 | 0.009 | |
| 419 | 0.14 | 0.005 | |
| 420 | ~0.39 | 0.29 | |
| 421 | 0.13 | <0.0018 | |
| 422 | ~0.27 | 0.088 | |
| 423 | 0.18 | 0.009 | |
| 424 | 0.17 | 0.004 | |
| 425 | 0.03 | 0.006 | |
| 426 | 0.09 | <0.0018 | |
| 427 | ~0.11 | <0.0018 | |
| 428 | 0.09 | 0.016 | |
| 429 | 0.17 | 0.0033 | |
| 430 | 0.11 | 0.0034 | |
| 431 | 0.54 | 0.079 | |
| 432 | 0.17 | 0.022 | |
| 433 | 0.15 | 0.002 | |
| 434 | 0.08 | 0.0029 | 2 |
| 435 | 0.17 | 0.003 | 2.47 |
| 436 | 0.36 | 0.0099 | 5.97 |
| 437 | 0.05 | 0.0033 | 4.7 |
| 438 | 0.16 | 0.004 | |
| 439 | 0.39 | 0.025 | |
| 440 | 13.68 | | |
| 441 | 0.92 | 0.68 | |
| 442 | 0.21 | 0.004 | |
| 443 | 0.08 | 0.004 | |
| 444 | 0.16 | | |
| 445 | 0.19 | 0.006 | |
| 446 | ~0.1 | 0.007 | |
| 447 | 0.15 | 0.004 | |
| 448 | 0.18 | 0.0046 | |
| 449 | 0.09 | 0.0084 | |
| 450 | 0.08 | 0.007 | |
| 451 | 0.15 | 0.011 | |
| 452 | 0.11 | 0.02 | |
| 453 | 0.05 | 0.006 | |
| 454 | 0.17 | 0.013 | |
| 455 | 0.14 | 0.024 | |
| 456 | 0.53 | | |
| 457 | 0.29 | | |
| 458 | 0.16 | 0.027 | |
| 459 | 0.06 | | |
| 460 | 0.07 | 0.011 | |
| 461 | 0.20 | 0.063 | |
| 462 | 0.27 | 0.089 | |
| 463 | 0.082 | 0.029 | |
| 478 | 0.29 | 0.012 | 1.25 |
| 479 | 1.77 | 0.53 | >15 |
| 480 | 0.15 | 0.006 | |
| 481 | 0.14 | >0.94 | |
| 482 | 0.18 | 0.015 | |
| 483 | 0.07 | 0.017 | |
| 488 | 0.15 | 0.004 | |
| 489 | 0.22 | | |
| 491a | 0.13 | 0.0049 | |
| 500 | 0.30 | 0.035 | 10.21 |
| 502 | 0.12 | 0.032 | |
| 503 | 0.18 | | |
| 504 | 0.58 | | |
| 505 | 0.54 | | |
| 512a | 29.7577 | 0.04 (1) | 0.31 |
| 512b | 0.589115 | >0.94 (1) | 5.81 |

1. A compound of Formula (I) 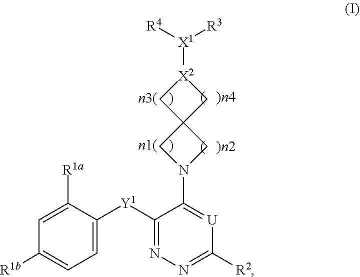
or a tautomer or a stereoisomeric form thereof, wherein
R^{1a} represents Het;
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl;
R^{1b} represents F or Cl;
Y¹ represents —CR^{5a}R^{5b}—, —O—, —S—, or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents C₁₋₅alkyl; 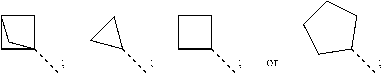
R^{5a}, R^{5b}, R^{5c}, R^{7a}, and R^{7b}, are each independently selected from the group consisting of hydrogen, C₁₋₄alkyl and C₃₋₆cycloalkyl;
R³ is selected from the group consisting of Het¹, Het², Cy² and —C₁₋₆alkyl-NR^{xc}R^{xd};
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano; or R^{xc} and R^{xd} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, and cyano;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of Het³; Het⁴; —C(═O)—NH—Cy¹; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂; or a bicyclic C-linked 6- to 11-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N;
wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{6b} and Het^{8b} each independently represent a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl; wherein said C₃₋₇cycloalkyl is optionally substituted with one, two, three or four substituents each independently selected from the group consisting of halo, R⁶, Het^{6a}, Het^{6b}, —NR^{9a}R^{9b}, —OH, C₁₋₄alkyl, and C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents Het⁵a; Het⁷; Het⁸a; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or
C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
or a pharmaceutically acceptable salt or a solvate thereof.

2. The compound according to claim 1, wherein
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of C₃₋₆cycloalkyl, cyano, and C₁₋₄alkyl;
R² is selected from the group consisting of hydrogen, halo, C₁₋₄alkyl, —O—C₁₋₄alkyl, and —NR^{7a}R^{7b};
R^{5a}, R^{5b}, R^{5c}, R^{7a}, and R^{7b}, are each independently selected from the group consisting of hydrogen, C₁₋₄alkyl and C₃₋₆cycloalkyl;
R³ is selected from the group consisting of Het¹, Het², Cy² and —C₁₋₆alkyl-NR^{xc}R^{xd};
R^{xc} represents Cy¹; Het⁵; —C₁₋₆alkyl-Cy₁; —C₁₋₆alkyl-Het³; —C₁₋₆alkyl-Het⁴; or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, and cyano;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶ and —C(═O)—R; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which is substituted on one nitrogen atom with R^{6a};
R⁶ is selected from the group consisting of Het³; —C(═O)—NH—R⁸;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R^{6a} represents C₁₋₆alkyl substituted with one substituent selected from the group consisting of —NR^{11a}R^{11b}, Het^{3a}, and Het^{6a};
R⁸ represents C₁₋₆alkyl optionally substituted with one, two or three substituents each independently selected from —OH, halo, cyano, —NR^{11a}R^{11b}, Het^{3a}, and Het^{6a};
Het³ and Het⁵ each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂; or a bicyclic C-linked 6- to 11-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het^{3a} and Het^{5a} each independently represent a monocyclic C-linked 4- to 7-membered fully saturated heterocyclyl containing one N-atom, and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom, and optionally one or two additional heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N; wherein said 5-membered aromatic ring is optionally substituted on one nitrogen atom with C₁₋₄alkyl; and wherein said 5- or 6-membered aromatic ring is optionally substituted on one carbon atom with —OH;
Het^{6a} and Het⁸ each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{1a} each independently represent a monocyclic N-linked 4- to 7-membered fully saturated heterocyclyl containing two N-atoms and optionally one additional heteroatom selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —(C═O)—NR^{10a}R^{10b}, —O—C₃₋₆cycloalkyl, —S(═O)₂—C₁₋₄alkyl, cyano, C₁₋₄alkyl, C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{6b} represents a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl substituted with one or two substituents each independently selected from the group consisting of —NR^{9a}R^{9b}; Het^{6a}; Het^{6b}; and C₁₋₆alkyl substituted with one or two substituents each independently selected from the group consisting of Het^{3a}, Het^{6a}, Het^{6b}, and —NR^{9a}R^{9b}; and said C₃₋₇cycloalkyl is optionally substituted with one or two additional substituents each independently selected from the group consisting of halo, R⁶, C₁₋₄alkyl, and —OH;
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; Het⁵; —C₁₋₄alkyl-R¹⁶; —C(═O)—C₁₋₄alkyl-Het^{3a}; —C(═O)—R¹⁴; C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het^{8a}; or
C₁₋₄alkyl substituted with one, two or three substituents selected from the group consisting of —NR^{13a}R^{13b} and Het^{8a};
R¹⁶ represents —C(═O)—NR^{17a}R^{17b}, —S(═O)₂—C₁₋₄alkyl, Het⁵, Het⁷, or Het⁸.

3. The compound according to claim 1, wherein
Het represents a monocyclic 5- or 6-membered aromatic ring containing one, two or three nitrogen atoms and optionally a carbonyl moiety; wherein said monocyclic 5- or 6-membered aromatic ring is substituted with one C₃₋₆cycloalkyl and wherein said monocyclic 5- or 6-membered aromatic ring is optionally substituted with one or two additional substituents selected from the group consisting of cyano, and C₁₋₄alkyl;
R^{1b} represents F;
Y¹ represents —O—;
R² represents hydrogen;
U represents N;
n1, n2, n3 and n4 are each independently selected from 1 and 2;
R⁴ represents C₁₋₅alkyl; or 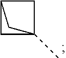
R³ is selected from the group consisting of Het¹ and Cy²;
Het¹ represents a monocyclic C-linked 4- to 7-membered fully saturated heterocyclyl containing one N-atom and optionally one or two additional heteroatoms each independently selected from O, S, and N, wherein said S-atom might be substituted to form S(═O) or S(═O)₂; or a bicyclic C-linked 6- to 11-membered fully saturated heterocyclyl containing one N-atom and optionally one or two additional heteroatoms each independently selected from O, S, and N, wherein said S-atom might be substituted to form S(═O) or S(═O)₂;
wherein said heterocyclyl is optionally substituted on one nitrogen with a substituent selected from the group consisting of R⁶ and —C(═O)—R; and wherein said heterocyclyl is optionally substituted on one or two carbon atoms with in total one, two, three or four substituents each independently selected from the group consisting of oxo and —OH;
R⁶ and R^{6a} are each independently selected from the group consisting of Het⁴; C₁₋₆alkyl optionally substituted with one or two substituents each independently selected from the group consisting of Het³, Het^{6a}, and Cy¹; and C₃₋₆cycloalkyl;
R⁸ represents —O—C₁₋₆alkyl;
Het³, Het^{3a}, Het⁵ and Het^{5a} each independently represent a monocyclic C-linked 4- to 7-membered fully saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N;
wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, and —O—C₁₋₄alkyl; and wherein said heterocyclyl is optionally substituted on one nitrogen with a substituent selected from the group consisting of —C(═O)—C₁₋₄alkyl and —(C═O)—NR^{10a}R^{10b};
Het^{6b} and Het^{8b} each independently represent a bicyclic N-linked 6- to 11-membered fully saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, and C₁₋₄alkyl;
Het⁹ represents a monocyclic C-linked 5- or 6-membered aromatic ring containing one, two or three heteroatoms each independently selected from O, S, and N; wherein said aromatic ring is optionally substituted on one or two carbon atoms with C₁₋₄alkyl;
Cy¹ represents C₃₋₆cycloalkyl optionally substituted with one, two or three substituents selected from the group consisting of —OH and C₁₋₄alkyl;
Cy² represents C₃₋₇cycloalkyl; wherein said C₃₋₇cycloalkyl is optionally substituted with one, two, three or four substituents each independently selected from the group consisting of R⁶, Het^{6a}, Het^{6b}, —NR^{9a}R^{9b}, —OH, and C₁₋₄alkyl;
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; Het⁵; Het⁷; —C₁₋₄alkyl-R¹⁶; —C(═O)—C₁₋₄alkyl-Het^{3a}; —C(═O)—R¹⁴; and
C₁₋₄alkyl substituted with one, two or three substituents selected from the group consisting of halo, —OH, and —O—C₁₋₄alkyl;
R^{11a}R^{11b}, R^{13a}, R^{13b}, R^{17a}, and R^{17b} are each independently selected from the group consisting of hydrogen and C₁₋₄alkyl;
R^{10a} and R^{10b} are each independently selected from the group consisting of hydrogen, C₁₋₄alkyl, and C₃₋₆cycloalkyl;
R¹⁴ represents —O—C₁₋₄alkyl; C₃₋₆cycloalkyl substituted with one, two or three substituents selected from the group consisting of —O—C₁₋₄alkyl and halo; or
C₁₋₄alkyl substituted with one, two or three substituents selected from the group consisting of —O—C₁₋₄alkyl, —NR^{13a}R^{13b}, and cyano;
R¹⁶ represents —C(═O)—NR^{17a}R^{17b} or —S(═O)₂—C₁₋₄alkyl.

4. The compound according to claim 1, wherein
Het represents

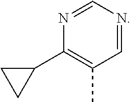

5. The compound according to claim 1, wherein U represents N.

6. The compound according to claim 1, wherein
Y¹ represents —O—.

7. The compound according to claim 1, wherein
R^{1b} represents F.

8. A pharmaceutical composition comprising a compound as claimed in claim 1 and a pharmaceutically acceptable carrier or diluent.

9. A process for preparing a pharmaceutical composition as defined in claim 8 comprising mixing a pharmaceutically acceptable carrier with a therapeutically effective amount of a compound according to claim 1.

10. A compound as claimed in claim 1 for use as a medicament.

11. A compound as claimed in claim 1 for use in the prevention or treatment of cancer, myelodysplastic syndrome (MDS) and diabetes.

12. The compound or a pharmaceutical composition for use according to claim 11, wherein cancer is selected from leukemias, myeloma or a solid tumor cancer such as prostate cancer, lung cancer, breast cancer, pancreatic cancer, colon cancer, liver cancer, melanoma and glioblastoma.

13. The compound or a pharmaceutical composition for use according to claim 12, wherein the leukemia is selected from acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.

14. A method of treating or preventing a disorder selected from cancer, myelodysplastic syndrome (MDS) and diabetes comprising administering to a subject in need thereof, a therapeutically effective amount of a compound as claimed in claim 1.

15. The method according to claim 13 wherein the disorder is cancer.

16. A pharmaceutical composition as claimed in claim 8 for use as a medicament.

17. A pharmaceutical composition as claimed in claim 8 for use in the prevention or treatment of cancer, myelodysplastic syndrome (MDS) and diabetes.

18. A method of treating or preventing a disorder selected from cancer, myelodysplastic syndrome (MDS) and diabetes comprising administering to a subject in need thereof, a therapeutically effective amount of a pharmaceutical composition as claimed in claim 8.
